(19) United States
(12) Patent Application Publication
(10) Pub. No.: US 2023/0406929 A1
(43) Pub. Date: Dec. 21, 2023

(54) POLYPEPTIDE CONSTRUCTS BINDING TO CD3

(71) Applicants: AMGEN INC., Thousand Oaks, CA (US); AMGEN RESEARCH (MUNICH), Munich (DE)

(72) Inventors: Doris Rau, Munich (DE); Tobias Raum, Munich 81477 (DE); Partrick Hoffmann, Munich (DE); Markus Muenz, Munich (AD); Matthias Klinger, Munich (DE); Virginie Naegele, Munich (DE); Lisa Winkel, Munich (DE); Pavan Ghattyvenkatakrishna, Thousand Oaks, CA (US); Joon Hoi Huh, Thousand Oaks, CA (US); Arnold McAuley, Thousand Oaks, CA (US); Sekhar Kanapuram, Thousand Oaks, CA (US)

(21) Appl. No.: 18/251,120

(22) PCT Filed: Nov. 8, 2021
(86) PCT No.: PCT/EP2021/080859

§ 371 Date: Apr. 28, 2023

Related U.S. Application Data
(60) Provisional application No. 63/110,545, filed on Nov. 6, 2020.

(51) Int. Cl. C07K 16/28

(52) U.S. Cl. CPC C07K 16/2809; C07K 16/2875; C07K 2317/31; C07K 2317/622; C07K 2317/73; C07K 2317/94; C07K 2317/92

(57) ABSTRACT

The invention relates to a polypeptide or polypeptide construct comprising: a binding domain binding to an extracellular epitope of the human CD3s chain comprising or consisting of a VH region and a VL region, wherein i) the VH region comprises: a CDR-H1 sequence of X₁YAX₂N, where X1 is K, V, S, G, R, T, or I; and X₂ is M or I; a CDR-H2 sequence of RIRSKYNNYATYYADX₁VKX₂, where X₁ is S or Q; and X₂ is D, G, K, S, or E; and a CDR-H3 sequence of HX₁NFGNSYX₂SX₃X₄AY, where X₁ is G, R, or A; X₂ is I, L, V, or T; X₃ is Y, W or F; and X₄ is W, F or Y; and ii) wherein the VL region comprises: a CDR-L1 sequence of X₁ SSTGAVTX₂X₃X₄YX₅N, where X₁ is G, R, or A; X₂ is S or T; X₃ is G or S; X₄ is N or Y and X₅ is P or A; a CDR-L2 sequence of X₁TX₂X₃X₄X₅X₆; where X₁ is G or A; X₂ is K, D, or N; X₃ is F, M or K; X₄ is L or R; X₅ is A, P, or V; and X₆ is P or S; and a CDR-L3 sequence of X₁LWYSNX₂VW, where X₁ is V, A, or T; and X₂ is R or L; and iii) wherein one or more of CDR sequences of the VH region of i) and/or of the VL region of ii) comprise one amino acid substitution or a combination thereof selected from X₂4V and X₂4F in CDR-H1; D15, and X₁16A in CDR-H2; H1, X₁2E, F4, and N6 in CDR-H3; and X₁1L and W3 in CDR-L3. The invention also relates to a polynucleotide encoding the polypeptide or polypeptide construct of the invention, a vector comprising said polynucleotide and a host cell transformed or transfected with said polynucleotide or with said vector. Moreover, the invention also provides for a process for the production of said polypeptide or polypeptide construct and a pharmaceutical composition comprising said polypeptide or polypeptide construct of the invention. Furthermore, the invention relates to medical uses of said polypeptide or polypeptide construct and kits comprising said polypeptide or polypeptide construct.

The invention relates to a polypeptide or polypeptide construct comprising: a binding domain binding to an extracellular epitope of the human CD3ε chain comprising or consisting of a VH region and a VL region, wherein i) the VH region comprises: a CDR-H1 sequence of X₁YAX₂N, where X, is K, V, S, G, R, T, or I; and X₂ is M or I; a CDR-H2 sequence of RIRSKYNNYATYYADX₁VKX₂, where X, is S or Q; and X₂ is D, G, K, S, or E; and a CDR-H3 sequence of HX₁NFGNSYX₂SX₃X₄AY, where X, is G, R, or A; X₂ is I, L, V, or T; X₃ is Y, W or F; and X₄ is W, F or Y; and ii) wherein the VL region comprises: a CDR-L1 sequence of X₁SSTGAVTX₂X₃X₄YX₅N, where X, is G, R, or A; X₂ is S or T; X₃ is G or S; X₄ is N or Y; and X₅ is P or A; a CDR-L2 sequence of X₁TX₂X₃X₄X₅X₆; where X, is G or A; X₂ is K, D, or N; X₃ is F, M or K; X₄ is L or R; X₅ is A, P, or V; and X₆ is P or S; and a CDR-L3 sequence of X₁LWYSNX₂WV, where X, is V, A, or T; and X₂ is R or L; and iii) wherein one or more of CDR sequences of the VH region of i) and/or of the VL region of ii) comprise one amino acid substitution or a combination thereof selected from X₂4V and X₂4F in CDR-H1; D15, and X₁16A in CDR-H2; H1, X₁2E, F4, and N6 in CDR-H3; and X₁1L and W3 in CDR-L3. The invention also relates to a polynucleotide encoding the polypeptide or polypeptide construct of the invention, a vector comprising said polynucleotide and a host cell transformed or transfected with said polynucleotide or with said vector. Moreover, the invention also provides for a process for the production of said polypeptide or polypeptide construct and a pharmaceutical composition comprising said polypeptide or polypeptide construct of the invention. Furthermore, the invention relates to medical uses of said polypeptide or polypeptide construct and kits comprising said polypeptide or polypeptide construct.

T cell engagers (TCEs) make use of T cells' ability to recognize foreign peptides on mutated or infected cells through T cell receptors. T cell recognition is mediated by clonotypically distributed alpha beta and gamma delta T cell receptors (TcR) that interact with the peptide-loaded molecules of the peptide MHC (pMHC) at low affinities. The antigen-specific chains of the TcR do not possess signalling domains but instead are coupled to the conserved multisubunit signaling apparatus CD3. The mechanism by which TcR ligation is directly communicated to the signalling apparatus is a fundamental topic of interest in T cell biology. In brief, CD3 subunits that translate cell surface antigen binding into an intracellular phosphorylation signaling cascade. These phosphorylation events culminate in the activation of transcription factors such as NFAT and NFkB that lead to increased expression of cytokines and effector proteins such as granzymes and perforin. It is this characteristic that is used by TCEs which bind simultaneously a target antigen on a tumor cell and CD3 on T cells to form an artificial immune synapse eventually leading to the destruction of the cell expressing said target antigen.

From a clinical perspective, TCEs do not only have to show efficient cell killing activity, but at the same time other characteristics like, e.g., a manageable side effect profile are desired. However, these aspects are not the only considerations when it comes to the development of TCEs. Other areas of importance for drug development are pre-patient, i.e. concern characteristics of the TCE outside of the patient's body such as, e.g., shelf life, production scalability, solubility, stability, and ease of formulation. In other terms, an important feature from a pharmaceutical perspective is the developability of a given TCE or parts thereof to address certain needs such as for example in drug production or administration. Bringing together the requirements from clinical and pharmacological perspective is important for TCE platform advancement in relation to commercialization. In view of the biological complexities of having the TCE inducing the formation of the immune synapse, it can be challenging to manipulate the TCE in terms of its other characteristics such as for example shelf life, production scalability, solubility, stability, and ease of formulation while maintaining its effectiveness in mediating the killing of target cells.

Thermal stability of TCEs is an important feature within and outside of the patient's body. A higher thermal stability of TCEs reduces the formation of high molecular weight aggregates caused which in turn provides, e. g., for a TCE that is active for a prolonged time in the body but will also be more stable during storage. Moreover, the reduction of high molecular weight aggregates can also result in a more favourable immunogenetic profile of the TCE upon administration thereby reducing the risk of side effects.

Classically, the VH and VL domains making up the anti-target and anti-CD3 binding domains of TCEs are an area that has been the focus of improving stability. This is in particular true for Fvs that are part of TCEs. The hydrophobic interaction between the VH and the VL is believed to strongly determine the stability of a Fv. The interaction can in many Fvs not be strong enough to be stable for in vitro and/or in vivo applications. This has led to the coupling of the V-domains of an Fv by a linker resulting in single chain Fvs (scFvs). While the linker has shown to be effective in holding the V-domains (variable domains) together, it may not always be effective to keep a scFv active at all or active enough for a given application making it necessary to further improve stability. One approach to increase stability is that by increasing the interaction of the two V-domains, the stability of a biologically active Fv can be increased so that it remains a target binding monomer. Although approaches have been described in the art to improve stability of scFv, there is still a need in the art for improving stability of Fvs that are part of TCEs.

The invention relates to a polypeptide or polypeptide construct comprising: a binding domain binding to an extracellular epitope of the human CD3ε chain comprising or consisting of a VH region and a VL region, wherein
i) the VH region comprises:
   a CDR-H1 sequence of X₁YAX₂N, where X, is K, V, S, G, R, T, or I; and X₂ is M or I;
   a CDR-H2 sequence of RIRSKYNNYATYYADX₁VKX₂, where X, is S or Q; and X₂ is D, G, K, S, or E; and
   a CDR-H3 sequence of HX₁NFGNSYX₂SX₃X₄AY, where X, is G, R, or A; X₂ is I, L, V, or T; X₃ is Y, W or F; and X₄ is W, F or Y; and
ii) wherein the VL region comprises:
   a CDR-L1 sequence of X₁SSTGAVTX₂X₃X₄YX₅N, where X, is G, R, or A; X₂ is S or T; X₃ is G or S; X₄ is N or Y; and X₅ is P or A;
   a CDR-L2 sequence of X₁TX₂X₃X₄X₅X₆; where X, is G or A; X₂ is K, D, or N; X₃ is F, M or K; X₄ is L or R; X₅ is A, P, or V; and X₆ is P or S; and
   a CDR-L3 sequence of X₁LWYSNX₂WV, where X, is V, A, or T; and X₂ is R or L; and
iii) wherein one or more CDR sequences of the VH region of i) and/or of the VL region of ii) comprise one or more amino acid substitutions selected from X₂4V and X₂4F in CDR-H1;
   D15 (preferably E), and X₁16A in CDR-H2;
   H1 (preferably A or N), X₁2E, F4 (preferably I), and N6 (preferably S or T) in CDR-H3; and
   X₁1L and W3 (preferably Y) in CDR-L3.

The term “polypeptide construct” (alternatively referred to also as “compound” herein) refers to an antigen-binding (or epitope-binding) molecule comprising domains themselves comprising paratopes. In the context of the present invention, a polypeptide construct is understood as an organic polymer which comprises at least one continuous, unbranched amino acid chain that naturally is not existing, but was engineered. An example of a polypeptide construct that is a single polypeptide is a BiTE® molecule that comprises a core structure comprising at least one functional target binding domain together with at least one complete functional CD3 binding domain on a single polypeptide chain, wherein these domains are linked directly by flexible peptide (a “linker”) without any further inserted domain unlike, for example, Xmabs that comprise the target binder and the CD3 binder on different polypeptide chains. In the context of the present invention, such a polypeptide construct comprising more than one amino acid chain is likewise envisaged. It is preferred that the term “polypeptide” is used in connection with single chain forms of the compounds of the present invention, whereas “polypeptide construct” may preferably be more adequate to describe also polypeptides that comprise more than one polypeptide chain, for example two, three or four polypeptide chains. Additionally, the term “polypeptide construct” is also suitable to describe compounds of the invention that comprise one or more non-amino acid-based constituents, e.g. human serum albumin, etc. (HSA). An amino acid chain of a polypeptide typically comprises at least 50 amino acids, preferably at least 100, 200, 300, 400 or 500 amino acids. It is also envisaged in the context of the present invention that an amino acid chain of a polymer is linked to an entity which is not composed of amino acids.

The polypeptides comprise structural and/or functional features based on the structure and/or function of an antibody, e.g., of a full-length immunoglobulin molecule. A polypeptide construct, hence, specifically and, preferably, selectively or immunospecifically binds to its target or antigen, more precisely to an epitope of said target or target antigen, and/or it comprises the heavy chain variable region (VH) and/or the light chain variable region (VL) naturally found in an antibody, or comprises domains derived therefrom. Accordingly, the constructs may alternatively be regarded as comprising paratope-structured and epitope-binding structures, such as those found in natural antibodies or fragments thereof. A polypeptide construct according to the invention comprises the minimum structural requirements of an antibody which allow for immunospecific target binding, i.e., a paratope that recognizes immunospecifically or immunoselectively an epitope on a target antigen unless specified differently. This minimum requirement may e.g. be defined by the presence of at least three light chain CDRs (i.e. CDR1, CDR2 and CDR3 of the VL region, also termed CDR-L1, CDRL2, and CDR-L3) and/or three heavy chain CDRs (i.e. CDR1, CDR2 and CDR3 of the VH region, also termed CDR-H1, CDR-H2 and CDR-H3), preferably of all six CDRs. A polypeptide construct may hence be characterized by the presence of three or six CDRs in a binding domain, and the skilled person knows where (in which order) those CDRs are located within the paratopic binding structures. In in the context of the CD3 binding domain of the polypeptide or polypeptide construct, said paratopic binding structure is specified to be a binding domain characterized by the presence of a VH and VL region comprising CDRs. Hence, a polypeptide/polypeptide construct according to the invention comprises at least a paratopic binding structure being a binding domain binding selectively, immunospecifically and/or immunoselectively to an extracellular epitope of the human CD3ε (also termed CD3epsilon or “CD3 epsilon” herein) chain comprising VH and VL regions with CDRs. Accordingly, a polypeptide/polypeptide construct according to the invention comprises a paratope selectively, immunospecifically and/or immunoselectively binding to an epitope of human CD3 epsilon. The terms “CDR”, and its plural “CDRs”, refer to the complementarity determining region of which three make up the binding character of a light chain variable region (CDR-L1, CDR-L2 and CDR-L3) and three make up the binding character of a heavy chain variable region (CDR-H1, CDR-H2 and CDR-H3). CDRs contain most of the residues responsible for specific interactions of antibodies (or constructs or binding domain) with an antigen and hence contribute to the functional activity of an antibody molecule: they are the main determinants of antigen specificity. The exact definition of CDR boundaries and lengths is subject to different classification and numbering systems. CDRs may therefore be referred to by Kabat, Chothia, contact or any other boundary definitions, including the numbering system described herein. Despite differing boundaries, each of these systems has some degree of overlap in what constitutes the so called “hypervariable regions” within the variable sequences. CDR definitions according to these systems may therefore differ in length and boundary areas with respect to the adjacent framework region. See for example Kabat (an approach based on cross-species sequence variability), Chothia (an approach based on crystallographic studies of antigen-antibody complexes), and/or MacCallum (Kabat et al., loc. cit.; Chothia et al., J. Mol. Biol, 1987, 196: 901-917; and MacCallum et al., J. Mol. Biol, 1996, 262: 732). Still another standard for characterizing the antigen binding site is the AbM definition used by Oxford Molecular's AbM antibody modeling software. See, e.g., Protein Sequence and Structure Analysis of Antibody Variable Domains. In: Antibody Engineering Lab Manual (Ed.: Duebel, S. and Kontermann, R., Springer-Verlag, Heidelberg). To the extent that two residue identification techniques define regions of overlapping, but not identical regions, they can be combined to define a hybrid CDR. However, the numbering in accordance with the so-called Kabat system is preferred The term “antigen-binding structure”, as used herein, refers to any polypeptide/polypeptide construct that comprises an antigen-binding structure or any molecule that has binding activity to a specified antigen. Said antigen-binding structures or molecules are not limited to those derived from a living organism, and for example, they may be a polypeptide produced from an artificially designed sequence. They may also be any of a naturally occurring polypeptide, synthetic polypeptide, recombinant polypeptide, and such. As the antigen-binding structure in accordance with the present invention bind specifically to parts of an antigen, i.e., they bind specifically to an epitope of CD3epsilon, the antigen (epitope)-binding structure may also be broadly defined as “paratopic structure” herein. Accordingly, the polypeptides/polypeptide constructs according to the invention may also be defined as a domain comprising a paratope that are preferably immunospecifically or immunoselectively binding to a target antigen/target epitope; and in certain embodiments comprising at least a further paratope, preferably immunospecifically or immunoselectively, binding to a further, different or same, target antigen/target epitope. Therefore, whenever the present description refers to a domain of a construct or molecule of the present invention, the construct comprises at least one paratopic structure (or paratope) binding human CD3epsilon as specified herein, particularly according to any one of the appended claims. In certain embodiments, said construct comprises at least a further paratope binding also to human CD3epsilon or a different target antigen as defined herein.

The term “antibody” as used in accordance with the invention comprises full-length antibodies, also including camelid antibodies and other immunoglobulins generated by biotechnological or protein engineering methods or processes. These full-length antibodies may be for example monoclonal, recombinant, chimeric, deimmunized, humanized and human antibodies, as well as antibodies from other species such as mouse, hamster, rabbit, rat, goat, or non-human primates.

“Polypeptides/polypeptide constructs” of the present invention may also comprise the structure of a full-length immunoglobulin as it occurs naturally. For example, they may comprise (at least) two full-length antibody heavy chains and two full-length antibody light chains. However, given that the polypeptides/polypeptide constructs according to the invention preferably comprise a linker linking the VH and VL region of the CD3 binding domain, preferably resulting in a scFv, and/or, in other embodiments, comprise at least one further binding domain comprising a paratope, they do not occur naturally, and they are markedly different in their function from naturally occurring products. A polypeptide or polypeptide construct of the invention is hence an artificial “hybrid” molecule comprising, preferably, an scFv and/or, in some embodiments, distinct paratopes/binding domains with different specificities and/or selectivities.

As indicated above, the polypeptides of the invention may comprise more than one polypeptide chain, i.e. polypeptides comprising two or more polypeptide chains are also subject to the present invention, particularly polypeptides forming a three-dimensional protein-like structure that allows for the immunospecific binding to CD3epsilon. Therefore, the definition of the term “polypeptide construct” includes molecules consisting of only one polypeptide chain as well as molecules consisting of two, three, four or more polypeptide chains, which chains can be either identical (homodimers, homotrimers or homo oligomers) or different (heterodimer, heterotrimer or heterooligomer). Examples for the above identified antibodies and their fragments, variants, derivatives and constructs derived therefrom are described inter alia in Harlow and Lane, Antibodies: A laboratory manual, CSHL Press (1988); Kontermann and Dübel, Antibody Engineering, Springer, 2nd ed. 2010; and Little, Recombinant Antibodies for Immunotherapy, Cambridge University Press 2009.

“Polypeptides/polypeptide constructs” of the present invention may also comprise fragments of full-length antibodies, such as VH, VHH, VL, (s)dAb, Fv, light chain (VL-CL), Fd (VH-CH1), heavy chain, Fab, Fab′, F(ab′)2 or “rIgG” (“half antibody” consisting of a heavy chain and a light chain), whereas not all of the foregoing fragments are applicable for the CD3epsilon binding domain since it is defined to comprise a VH and a VL region, but to the embodiments regarding the at least one further binding domain. Polypeptides/polypeptide constructs according to the invention may also comprise modified fragments of antibodies, also called antibody variants or antibody derivatives. Examples include, but are not limited to, scFv, di-scFv or bi(s)-scFv, scFv-Fc, scFv-zipper, scFab, Fab2, Fab3, diabodies, single chain diabodies, tandem diabodies (Tandab's), tandem di-scFv, tandem tri-scFv “minibodies” exemplified by a structure which is as follows: (VH-VL-CH3)2, (scFv-CH3)2, ((scFv)2-CH3+CH3), ((scFv)2-CH3) or (scFv-CH3-scFv)2, multibodies such as triabodies or tetrabodies, and single domain antibodies such as nanobodies or single variable domain antibodies comprising merely one variable region, which might be VHH, VH or VL, that selectively and, preferably, specifically binds to an antigen or target independently of other variable regions or domains, whereas not all of the foregoing fragments are applicable for the CD3epsilon binding domain since it is defined to comprise a VH and a VL region, but to the embodiments regarding the at least one further binding domain. Further possible formats of the polypeptides/polypeptide constructs according to the invention are cross bodies, maxi bodies, hetero Fc constructs, mono Fc constructs and scFc constructs. Examples for those formats will be described herein below.

Furthermore, the definition of the term “polypeptide construct” includes bivalent and polyvalent/multivalent polypeptides/polypeptide constructs as well as bispecific and polyspecific/multispecific polypeptides/polypeptide constructs, which selectively and, preferably, specifically bind to two, three or more antigenic structures (epitopes), through distinct binding domains. A polypeptide construct can have more binding valences than specificities, e.g. in a case where it has two binding domains for one target (CD3epsilon) and one binding domain for another target, for example those described herein below, or vice versa, in which case the polypeptide construct is trivalent and bispecific. In general, the term “bispecific” includes the meaning that a polypeptide construct binds to at least two different antigens, such as said CD3epsilon and a further target, for example those specified herein below.

The terms “paratope”, “antigen-binding domain”, “epitope-binding domain”, “binding domain” or “domain which binds to . . . ” characterize, in connection with the present invention, a domain of the construct which selectively and, preferably, specifically or immunospecifically binds to/interacts with/recognizes an epitope on the target or antigen (here: CD3). The terms “binding domain” or “domain which binds to . . . ” or “domain” as far as it relates to the herein described “constructs” characterizes in connection with the present invention a domain of the construct which immunospecifically binds to/interacts with/recognizes an epitope on the target or antigen. The structure and function of the CD3epsilon binding domain (also termed as first binding domain in the case of a polypeptide/polypeptide construct comprising a further, consequently second, third, and so on, binding domain), and preferably also the structure and/or function of any further binding domain (binding to for example to a cell surface antigen such as a tumor antigen), is/are based on the structure and/or function of an antibody, e.g. of a full-length immunoglobulin polypeptide. The “binding domain” or “domain which binds to . . . ” may hence comprise the minimum structural requirements of an antibody which allow for immunospecific target binding. While the structural requirements of the CD3epsilon binding domain is specified to comprise a VH and VL region with corresponding three CDRs per region, said minimum structural requirement in any further binding domain may e.g. be defined by the presence of at least three light chain CDRs (i.e. CDR1, CDR2 and CDR3 of the VL region) and/or of three heavy chain CDRs (i.e. CDR1, CDR2 and CDR3 of the VH region), preferably of all six CDRs. A “domain which binds to” (or a “binding domain”) may typically comprise an antibody light chain variable region (VL) and an antibody heavy chain variable region (VH); however, it does not have to comprise both, but may comprise only one of VH or VL. Fd fragments, for example, often retain some antigen-binding function of the intact antigen-binding domain. The terms “paratope”, “antigen-binding structure” and “epitope-binding structure, as used herein, refer also to a portion of an antibody (or a molecule according to the invention), which comprises a region that specifically binds and is complementary to the whole or a portion of an antigen or a part thereof, i.e. an antibody can only bind to a particular portion of the antigen. The particular portion is called “epitope”. An antigen-binding domain can be provided from one or more antibody variable domains. Preferably, the antigen-binding domains contain antibody variable region that comprising both the antibody light chain variable region (VL) and antibody heavy chain variable region (VH). Such preferable antigen-binding domains include, for example, “single-chain Fv (scFv)”, “single-chain antibody”, “Fv”, “single-chain Fv2 (scFv2)”, “Fab”, and “F (ab”)2 “. For the CD3epsilon binding domain it is preferred that it takes the form of an scFv.

Examples for the format of a “domain which binds to”, “domain comprising a paratope” (or “binding domain”, “antigen-binding structure”, “epitope-binding structure”) include unless otherwise defined, but are not limited to, full-length antibodies, fragments of full-length antibodies (such as VH, VHH, VL), (s)dAb, Fv, light chain (VL-CL), Fd (VH-CH1), heavy chain, Fab, Fab′, F(ab′)2 or “r IgG” (“half antibody”)), antibody variants or derivatives such as scFv, di-scFv or bi(s)-scFv, scFv-Fc, scFv-zipper, scFab, Fab2, Fab3, diabodies, single chain diabodies, tandem diabodies (Tandab's), tandem di-scFv, tandem tri-scFv, “minibodies” (selected from formats such as (VH-VL-CH3)2, (scFv-CH3)2, ((scFv)2-CH3+CH3)), ((scFv)2-CH3) or (scFv-CH3-scFv)2, multibodies such as triabodies or tetrabodies, and single domain antibodies such as nanobodies or single variable domain antibodies comprising merely one variable region, which might be VHH, VH or VL. Further examples for the format of a “domain which binds to” (or a “binding domain”) include (1) an antibody fragment or variant comprising VL, VH, CL and CH1 (such as Fab); (2) an antibody fragment or variant comprising two linked Fab fragments (such as a F(ab′)2); (3) an antibody fragment or variant comprising VH and CH1 (such as Fd); (4) an antibody fragment or variant comprising VL and CL (such as the light chain); (5) an antibody fragment or variant comprising VL and VH (such as Fv); (5) a dAb fragment (Ward et al., (1989) Nature 341:544-546), which has a VH domain; (6) an antibody variant comprising at least three isolated CDRs of the heavy and/or the light chain; and (7) a single chain Fv (scFv). Examples for embodiments of constructs or binding domains according to the invention are e.g. described in WO 00/006605, WO 2005/040220, WO 2008/119567, WO 2010/037838, WO 2013/026837, WO 2013/026833, US 2014/0308285, US 2014/0302037, W O2014/144722, WO 2014/151910, and WO 2015/048272. In the context of the present invention, a paratope is understood as an antigen-binding site which is a part of a polypeptide as described herein and which recognizes and binds to an antigen. A paratope is typically a small region of about at least 5 amino acids. A paratope as understood herein typically comprises parts of antibody-derived heavy (VH) and light chain (VL) sequences. Each binding domain of a polypeptide according to the present invention is provided with a paratope comprising a set of 6 complementarity-determining regions (CDR loops) with three of each being comprised within the antibody-derived VH and VL sequence, respectively.

It is envisaged for the compounds, particularly for the constructs of the present invention that a) the construct is a single chain polypeptide or a single chain construct, b) the CD3epsilon binding domain is in the format of an scFv, c) any further, such as a second binding and/or third domain is in the format of an scFv, d) the first and said further, such as said second and/or third domain are connected via a linker, preferably a peptide linker, more preferably a glycine/serine or glycine/glutamine linker, and/or e) the construct comprises a domain providing an extended serum half-life, such as an Fc-based domain, or human serum albumin (HSA). In the latter case, it is a preferred embodiment, wherein the term “polypeptide construct” makes clear that it comprises more than a single peptide chain. A preferred Fc-based domain which extends the serum half-life (also termed “HLE” domain) comprises two polypeptide monomers, each comprising a hinge, a CH2 domain and a CH3 domain, wherein said two polypeptide monomers are fused to each other via a peptide linker (see, e.g., SEQ ID NO: 18 and 19); the format is in N-terminal to C-terminal order: hinge-CH2-CH3-linker-hinge-CH2-CH3.

The constructs of the present invention are preferably “in vitro generated constructs” and/or “recombinant constructs”. In the context of the present invention, the term “in vitro generated” refers to a construct according to the above definition where all or part of the binding domain or of a variable region (e.g., at least one CDR) is generated in a non-immune cell selection, e.g., in an in vitro phage display, on a protein chip or in any other method in which candidate amino acid sequences can be tested for their ability to bind to an antigen. This term thus preferably excludes sequences generated solely by genomic rearrangement in an immune cell in an animal. It is envisaged that the first and/or second domain of the construct is produced by or obtainable by phage display or library screening methods rather than by grafting CDR sequences from a pre-existing (monoclonal) antibody into a scaffold. A “recombinant construct” is a construct generated or produced using (inter alia) recombinant DNA technology or genetic engineering.

The constructs of the present invention are envisaged to be monoclonal. As used herein, polypeptides or constructs that are denominated “monoclonal” (mAb) are obtained from a population of substantially homogeneous antibodies/constructs, i.e., the individual antibodies/constructs comprised in the population are identical (in particular with respect to their amino acid sequence) except for possible naturally occurring mutations and/or post-translational modifications (e.g., isomerizations, amidations) that may be present in minor amounts. Monoclonal antibodies/constructs are highly specific, being directed against a single epitope within the antigen, in contrast to polyclonal antibody preparations which typically include different antibodies directed against different determinants (or epitopes). In addition to their specificity, monoclonal antibodies are advantageous in that they are synthesized by the hybridoma culture, hence uncontaminated by other immunoglobulins. The modifier “monoclonal” indicates the character of the antibody/construct as being obtained from a substantially homogeneous population of antibodies and is not to be construed as requiring production of the antibody by any specific method.

For the preparation of monoclonal antibodies, any technique providing antibodies produced by continuous cell line cultures can be used. For example, monoclonal antibodies to be used may be made by the hybridoma method first described by Koehler et al., Nature, 256: 495 (1975), or may be made by recombinant DNA methods (see, e.g., U.S. Pat. No. 4,816,567). Examples for further techniques to produce human monoclonal antibodies include the trioma technique, the human B-cell hybridoma technique (Kozbor, Immunology Today 4 (1983), 72) and the EBV-hybridoma technique (Cole et al., Monoclonal Antibodies and Cancer Therapy, Alan R. Liss, Inc. (1985), 77-96).

Hybridomas can then be screened using standard methods, such as enzyme-linked immunosorbent assay (ELISA) and surface plasmon resonance (BIACORE™) analysis, to identify one or more hybridomas that produce an antibody that selectively and, preferably, specifically or immunospecifically binds to a specified antigen. Any form of the relevant antigen may be used as the immunogen, e.g., recombinant antigen, naturally occurring forms, any variants or fragments thereof, as well as an antigenic peptide thereof. Surface plasmon resonance as employed in the BIAcore™ system can be used to increase the efficiency of phage antibodies/constructs which bind to an epitope of a target antigen (Schier, Human Antibodies Hybridomas 7 (1996), 97-105; Malmborg, J. Immunol. Methods 183 (1995), 7-13).

Another exemplary method of making constructs or binding domains includes screening protein expression libraries, e.g., phage display or ribosome display libraries. Phage display is described, for example, in Ladner et al., U.S. Pat. No. 5,223,409; Smith (1985) Science 228:1315-1317, Clackson et al., Nature, 352: 624-628 (1991) and Marks et al., J. Mol. Biol., 222: 581-597 (1991).

In addition to the use of display libraries, the relevant antigen can be used to immunize a non-human animal, e.g., a rodent (such as a mouse, hamster, rabbit or rat). In one embodiment, the non-human animal includes at least a part of a human immunoglobulin gene. For example, it is possible to engineer mouse strains deficient in mouse antibody production with large fragments of the human Ig (immunoglobulin) loci. Using the hybridoma technology, antigen-specific monoclonal antibodies derived from the genes with the desired specificity may be produced and selected. See, e.g., Xenomouse™ mouse, Green et al. (1994) Nature Genetics 7:13-21, US 2003-0070185, WO 96/34096, and WO 96/33735.

A monoclonal antibody can also be obtained from a non-human animal, and then modified, e.g., humanized, deimmunized, rendered chimeric etc., using recombinant DNA techniques known in the art. Examples of modified constructs or binding domains include humanized variants of non-human antibodies/constructs, “affinity matured” constructs or binding domains (see, e.g. Hawkins et al. J. Mol. Biol. 254, 889-896 (1992) and Lowman et al., Biochemistry 30, 10832-10837 (1991)) and antibody variants or mutants with altered effector function(s) (see, e.g., U.S. Pat. No. 5,648,260, Kontermann and Dubel (2010), loc. cit. and Little (2009), loc. cit.).

In immunology, affinity maturation is the process by which B cells produce antibodies with increased affinity for antigen during the course of an immune response. With repeated exposures to the same antigen, a host will produce antibodies of successively greater affinities. Like the natural prototype, the in vitro affinity maturation is based on the principles of mutation and selection. The in vitro affinity maturation has successfully been used to optimize antibodies, antibody fragments, antibody variants, constructs or binding domains. Random mutations inside the CDRs are introduced using radiation, chemical mutagens or error-prone PCR. In addition, the genetic diversity can be increased by chain shuffling. Two or three rounds of mutation and selection using display methods like phage display usually results in antibodies, antibody fragments, antibody variants, constructs or binding domains with affinities in the low nanomolar range.

A preferred type of an amino acid substitutional variation of the constructs or binding domains of the invention involves substituting one or more residues within the hypervariable region of a parent antibody structure (e.g. a humanized or human antibody structure). Generally, the resulting variant(s) selected for further development will have improved biological properties relative to the parent antibody structure from which they are generated. A convenient way for generating such substitutional variants involves affinity maturation using phage display. Briefly, several sites of the hypervariable region (e. g. 6-7 sites) are mutated to generate all possible amino acid substitutions at each site. The variants thus generated are displayed in a monovalent fashion from filamentous phage particles as fusions to the gene Ill product of M13 packaged within each particle. The phage-displayed variants are then screened for their biological activity (e.g. binding affinity) as disclosed herein. To identify candidate hypervariable region sites contributing significantly to antigen binding (candidates for modification), alanine scanning mutagenesis can also be performed. Alternatively, or additionally, it may be beneficial to analyze a crystal structure of the complex between the antigen and the construct or the binding domain to identify contact points between the binding domain and its specific antigen. Such contact residues and neighbouring residues are candidates for substitution according to the techniques elaborated herein. Once such variants are generated, the panel of variants is subjected to screening as described herein and antibodies, their antigen-binding fragments, constructs or binding domains with superior properties in one or more relevant assays may be selected for further development.

The constructs and binding domains of the present invention specifically include “chimeric” versions in which a portion of the heavy and/or light chain is identical with or homologous to corresponding sequences in antibodies derived from a particular species or belonging to a particular antibody class or subclass, while the remainder of the chain(s) is/are identical with or homologous to corresponding sequences in antibodies derived from another species or belonging to another antibody class or subclass, as well as fragments or variants of such antibodies, so long as they exhibit the desired biological activity (U.S. Pat. No. 4,816,567; Morrison et al., Proc. Natl. Acad. Sci. USA, 81: 6851-6855 (1984)). Chimeric constructs or binding domains of interest herein include “primitized” constructs comprising variable domain antigen-binding sequences derived from a non-human primate (e.g., Old World Monkey, Ape etc.) and human constant region sequences. A variety of approaches for making chimeric antibodies or constructs have been described. See e.g., Morrison et al., Proc. Natl. Acad. ScL U.S.A. 81:6851, 1985; Takeda et al., Nature 314:452, 1985, Cabilly et al., U.S. Pat. No. 4,816,567; Boss et al., U.S. Pat. No. 4,816,397; Tanaguchi et al., EP 0171496; EP 0173494; and GB 2177096.

An antibody, polypeptide construct, antibody fragment, antibody variant or binding domain may also be modified by specific deletion of human T cell epitopes (a method called “deimmunization”) using methods disclosed for example in WO 98/52976 or WO 00/34317. Briefly, the heavy and light chain variable regions of an antibody, construct or binding domain can be analyzed for peptides that bind to MHC class II; these peptides represent potential T cell epitopes (as defined e.g. in WO 98/52976 and WO 00/34317). For detection of potential T cell epitopes, a computer modeling approach termed “peptide threading” can be applied, and in addition a database of human MHC class II binding peptides can be searched for motifs present in the VH and VL sequences, as described in WO 98/52976 and WO 00/34317. These motifs bind to any of the 18 major MHC class II DR allotypes, and thus constitute potential T cell epitopes. Potential T cell epitopes detected can be eliminated by substituting small numbers of amino acid residues in the variable domains or variable regions, or preferably, by single amino acid substitutions. Typically, conservative substitutions are made. Often, but not exclusively, an amino acid common to a position in human germline antibody sequences may be used. Human germline sequences are disclosed e.g. in Tomlinson, et al. (1992) J. Mol. Biol. 227:776-798; Cook, G. P. et al. (1995) Immunol. Today Vol. 16 (5): 237-242; and Tomlinson et al. (1995) EMBO J. 14: 14:4628-4638. The V BASE directory (www2.mrc-Imb.cam.ac.uk/vbase/list2.php) provides a comprehensive directory of human immunoglobulin variable region sequences (compiled by Tomlinson, L A. et al. MRC Centre for Protein Engineering, Cambridge, UK). These sequences can be used as a source of human sequence, e.g., for framework regions and CDRs. Consensus human framework regions can also be used, for example as described in U.S. Pat. No. 6,300,064.

“Humanized” antibodies, variants or fragments thereof, constructs and binding domains are based on immunoglobulins of mostly human sequences, which contain (a) minimal sequence(s) derived from non-human immunoglobulin. For the most part, humanized antibodies, variants or fragments thereof, constructs and binding domains are based on human immunoglobulins (recipient antibodies) in which residues from a hypervariable region or CDR are replaced by residues from a hypervariable region or CDR of a non-human species (donor antibody) such as a rodent (e.g. mouse, hamster, rat or rabbit) having the desired specificity, affinity, capacity and/or biological activity. In some instances, Fv framework region (FR) residues of the human immunoglobulin are replaced by corresponding non-human residues. Furthermore, “humanized” antibodies, variants or fragments thereof, constructs and binding domains as used herein may also comprise residues which are found neither in the recipient antibody nor the donor antibody. These modifications are made to further refine and optimize antibody performance. The humanized antibodies, variants or fragments thereof, constructs and binding domains may also comprise at least a portion of an immunoglobulin constant region (such as Fc), typically that of a human immunoglobulin. For further details, see Jones et al., Nature, 321: 522-525 (1986); Reichmann et al., Nature, 332: 323-329 (1988); and Presta, Curr. Op. Struct. Biol., 2: 593-596 (1992).

Humanized antibodies, variants or fragments thereof, constructs and binding domains can be generated by replacing sequences of the (Fv) variable region that are not directly involved in antigen binding with equivalent sequences from human (Fv) variable regions. Exemplary methods for generating such molecules are provided by Morrison (1985) Science 229:1202-1207; by Oi et al. (1986) BioTechniques 4:214; and by U.S. Pat. Nos. 5,585,089; 5,693,761; 5,693,762; 5,859,205; and 6,407,213. These methods include isolating, manipulating, and expressing the nucleic acid sequences that encode all or part of immunoglobulin (Fv) variable regions from at least one of a heavy or light chain. Such nucleic acids may be obtained from a hybridoma producing an antibody against a predetermined target, as described above, as well as from other sources. The recombinant DNA encoding the humanized antibody, variant or fragment thereof, construct or binding domain can then be cloned into an appropriate expression vector.

Humanized antibodies, variants or fragments thereof, constructs and binding domains may also be produced using transgenic animals such as mice that express human heavy and light chain genes but are incapable of expressing the endogenous mouse immunoglobulin heavy and light chain genes. Winter describes an exemplary CDR grafting method that may be used to prepare the humanized molecules described herein (U.S. Pat. No. 5,225,539). All the CDRs of a given human sequence may be replaced with at least a portion of a non-human CDR, or only some of the CDRs may be replaced with non-human CDRs. It is only necessary to replace the number of CDRs required for binding of the humanized molecule to a predetermined antigen.

A humanized antibody, variant or fragment thereof, construct or binding domain can be optimized by the introduction of conservative substitutions, consensus sequence substitutions, germline substitutions and/or back mutations. Such altered immunoglobulin molecules can be made by any of several techniques known in the art, (e.g., Teng et al., Proc. Natl. Acad. Sci. U.S.A., 80: 7308-7312, 1983; Kozbor et al., Immunology Today, 4: 7279, 1983; Olsson et al., Meth. Enzymol., 92: 3-16, 1982, and EP 239 400).

Human anti-mouse antibody (HAMA) responses have led the industry to prepare chimeric or otherwise humanized antibodies/constructs. It is however expected that certain human anti-chimeric antibody (HACA) responses will be observed, particularly in chronic or multi-dose utilizations of an antibody or construct. Thus, it would be desirable to provide constructs comprising a human binding domain against a target, to vitiate concerns and/or effects of HAMA or HACA response.

Therefore, according to one embodiment, the polypeptide construct having at least one further binding domain, said binding domain(s) are “human”. The term “human antibody”, “human construct” and “human binding domain” includes antibodies, constructs and binding domains, respectively, having antibody-derived regions such as variable and constant regions or domains which correspond substantially to human germline immunoglobulin sequences known in the art, including, for example, those described by Kabat et al. (1991) (loc. cit.). The human constructs or binding domains of the invention may include amino acid residues not encoded by human germline immunoglobulin sequences (e.g., mutations introduced by random or site-specific mutagenesis in vitro or by somatic mutation in vivo), for example in the CDRs, and particularly in CDR3. The human constructs or binding domains can have at least one, two, three, four, five, or more positions replaced with an amino acid residue that is not encoded by the human germline immunoglobulin sequence. The definition of human antibodies, constructs and binding domains as used herein also contemplates fully human antibodies, constructs and binding domains which include only non-artificially and/or genetically altered human sequences of antibodies as those can be derived by using technologies or systems such as the XenomouseT mouse.

Polypeptides/polypeptide constructs comprising at least one human binding domain may avoid some of the problems associated with antibodies or constructs that possess non-human such as rodent (e.g. murine, rat, hamster or rabbit) variable and/or constant regions. The presence of such rodent derived proteins can lead to the rapid clearance of the antibodies or constructs or can lead to the generation of an immune response against the antibody or construct by a patient. To avoid the use of rodent-derived constructs, humanized or fully human constructs can be generated through the introduction of human antibody function into a rodent so that the rodent produces fully human antibodies.

The ability to clone and reconstruct megabase-sized human loci in YACs and to introduce them into the mouse germline provides a powerful approach to elucidating the functional components of very large or crudely mapped loci as well as generating useful models of human disease. Furthermore, the use of such technology for substitution of mouse loci with their human equivalents could provide unique insights into the expression and regulation of human gene products during development, their communication with other systems, and their involvement in disease induction and progression.

An important practical application of such a strategy is the “humanization” of the mouse humoral immune system. Introduction of human immunoglobulin (Ig) loci into mice in which the endogenous Ig genes have been inactivated offers the opportunity to study the mechanisms underlying programmed expression and assembly of antibodies as well as their role in B-cell development. Furthermore, such a strategy could provide an ideal source for production of fully human monoclonal antibodies (mAbs)—an important milestone towards fulfilling the promise of antibody therapy in human disease. Fully human antibodies or constructs derived therefrom are expected to minimize the immunogenic and allergic responses intrinsic to mouse or mouse-derivatized mAbs and thus to increase the efficacy and safety of the administered antibodies/constructs. The use of fully human antibodies or constructs can be expected to provide a substantial advantage in the treatment of chronic and recurring human diseases, such as inflammation, autoimmunity, and cancer, which require repeated compound administrations.

One approach towards this goal was to engineer mouse strains deficient in mouse antibody production with large fragments of the human Ig loci in anticipation that such mice would produce a large repertoire of human antibodies in the absence of mouse antibodies. Large human Ig fragments would preserve the large variable gene diversity as well as the proper regulation of antibody production and expression. By exploiting the mouse machinery for antibody diversification and selection and the lack of immunological tolerance to human proteins, the reproduced human antibody repertoire in these mouse strains should yield high affinity antibodies against any antigen of interest, including human antigens. Using the hybridoma technology, antigen-specific human mAbs with the desired specificity could be readily produced and selected. This general strategy was demonstrated in connection with the generation of the first XenoMouse™ mouse strains (see Green et al. Nature Genetics 7:13-21 (1994)). The XenoMouse strains were engineered with yeast artificial chromosomes (YACs) containing 245 kb and 190 kb-sized germline configuration fragments of the human heavy chain locus and kappa light chain locus, respectively, which contained core variable and constant region sequences. The human Ig containing YACs proved to be compatible with the mouse system for both rearrangement and expression of antibodies and were capable of substituting for the inactivated mouse Ig genes. This was demonstrated by their ability to induce B cell development, to produce an adult-like human repertoire of fully human antibodies, and to generate antigen-specific human mAbs. These results also suggested that introduction of larger portions of the human Ig loci containing greater numbers of V genes, additional regulatory elements, and human Ig constant regions might recapitulate substantially the full repertoire that is characteristic of the human humoral response to infection and immunization. The work of Green et al. was extended to the introduction of greater than approximately 80% of the human antibody repertoire through introduction of megabase sized, germline configuration YAC fragments of the human heavy chain loci and kappa light chain loci, respectively. See Mendez et al. Nature Genetics 15:146-156 (1997) and U.S. patent application Ser. No. 08/759,620.

The production of the XenoMouse™ model is further discussed and delineated in U.S. patent application Ser. No. 07/466,008, Ser. No. 07/610,515, Ser. No. 07/919,297, Ser. No. 07/922,649, Ser. No. 08/031,801, Ser. No. 08/112,848, Ser. No. 08/234,145, Ser. No. 08/376,279, Ser. No. 08/430,938, Ser. No. 08/464,584, Ser. No. 08/464,582, Ser. No. 08/463,191, Ser. No. 08/462,837, Ser. No. 08/486,853, Ser. No. 08/486,857, Ser. No. 08/486,859, Ser. No. 08/462,513, Ser. No. 08/724,752, Ser. No. 08/759,620; and U.S. Pat. Nos. 6,162,963; 6,150,584; 6,114,598; 6,075,181, and 5,939,598 and Japanese Patent Nos. 3 068 180 B2, 3 068 506 B2, and 3 068 507 B2. See also Mendez et al. Nature Genetics 15:146-156 (1997) and Green and Jakobovits J. Exp. Med. 188:483-495 (1998), EP 0 463 151 B1, WO94/02602, WO96/34096, WO98/24893, WO 00/76310, and WO 03/47336.

In an alternative approach, others, including GenPharm International, Inc., have utilized a “minilocus” approach. In the minilocus approach, an exogenous Ig locus is mimicked through the inclusion of pieces (individual genes) from the Ig locus. Thus, one or more VH genes, one or more DH genes, one or more JH genes, a mu constant region, and a second constant region (preferably a gamma constant region) are formed into a construct for insertion into an animal. This approach is described in U.S. Pat. No. 5,545,807 to Surani et al. and U.S. Pat. Nos. 5,545,806; 5,625,825; 5,625,126; 5,633,425; 5,661,016; 5,770,429; 5,789,650; 5,814,318; 5,877,397; 5,874,299; and 6,255,458 each to Lonberg and Kay, U.S. Pat. Nos. 5,591,669 and 6,023,010 to Krimpenfort and Berns, U.S. Pat. Nos. 5,612,205; 5,721,367; and U.S. Pat. No. 5,789,215 to Berns et al., and U.S. Pat. No. 5,643,763 to Choi and Dunn, and GenPharm International U.S. patent application Ser. No. 07/574,748, Ser. No. 07/575,962, Ser. No. 07/810,279, Ser. No. 07/853,408, Ser. No. 07/904,068, Ser. No. 07/990,860, Ser. No. 08/053,131, Ser. No. 08/096,762, Ser. No. 08/155,301, Ser. No. 08/161,739, Ser. No. 08/165,699, Ser. No. 08/209,741. See also EP 0 546 073 B1, WO 92/03918, WO 92/22645, WO 92/22647, WO 92/22670, WO 93/12227, WO 94/00569, WO 94/25585, WO 96/14436, WO 97/13852, and WO 98/24884 and U.S. Pat. No. 5,981,175. See further Taylor et al. (1992), Chen et al. (1993), Tuaillon et al. (1993), Choi et al. (1993), Lonberg et al. (1994), Taylor et al. (1994), and Tuaillon et al. (1995), Fishwild et al. (1996).

Kirin has also demonstrated the generation of human antibodies from mice in which, through microcell fusion, large pieces of chromosomes, or entire chromosomes, have been introduced. See European Patent Application Nos. 773 288 and 843 961. Xenerex Biosciences is developing a technology for the potential generation of human antibodies. In this technology, SCID mice are reconstituted with human lymphatic cells, e.g., B and/or T cells. Mice are then immunized with an antigen and can generate an immune response against the antigen. See U.S. Pat. Nos. 5,476,996; 5,698,767; and 5,958,765.

In some embodiments, the constructs of the invention are “isolated” or “substantially pure” constructs. “Isolated” or “substantially pure”, when used to describe the constructs disclosed herein, means a construct that has been identified, separated and/or recovered from a component of its production environment. Preferably, the construct is free or substantially free of association with all other components from its production environment. Contaminant components of its production environment, such as that resulting from recombinant transfected cells, are materials that could interfere with diagnostic or therapeutic uses for the construct, and may include enzymes, hormones, and other proteinaceous or non-proteinaceous compounds. It is understood that the isolated or substantially pure construct may constitute from 5% to 99.9% by weight of the total protein/polypeptide content in a given sample, depending on the circumstances. The desired construct may be produced at a significantly higher concentration using an inducible promoter or high expression promoter. The definition includes the production of a construct in a wide variety of organisms and/or host cells that are known in the art. In certain embodiments, the construct will be purified (1) to a degree sufficient to obtain at least 15 residues of N-terminal or internal amino acid sequence by use of a spinning cup sequenator, or (2) to homogeneity by SDS-PAGE under non-reducing or reducing conditions using Coomassie Blue or, preferably, silver staining. Usually, however, an isolated construct will be prepared by at least one purification step.

According to one embodiment, the entire construct and/or the binding domains are in the form of one or more polypeptides or in the form of proteins. In addition to proteinaceous parts, such polypeptides or proteins may include non-proteinaceous parts (e.g. chemical linkers or chemical cross-linking agents such as glutaraldehyde).

Peptides are short chains of amino acid monomers linked by covalent peptide (amide) bonds. Hence, peptides fall under the broad chemical classes of biological oligomers and polymers. Amino acids that are part of a peptide or polypeptide chain are termed “residues” and can be consecutively numbered. All peptides except cyclic peptides have an N-terminal residue at one end and a C-terminal residue at the other end of the peptide. An oligopeptide consists of only a few amino acids (usually between two and twenty). A polypeptide is a longer, continuous, and unbranched peptide chain. Peptides are distinguished from proteins based on size, and as an arbitrary benchmark can be understood to contain approximately 50 or fewer amino acids. Proteins consist of one or more polypeptides, usually arranged in a biologically functional way. While aspects of the lab techniques applied to peptides versus polypeptides and proteins differ (e.g., the specifics of electrophoresis, chromatography, etc.), the size boundaries that distinguish peptides from polypeptides and proteins are not absolute. Therefore, in the context of the present invention, the terms “peptide”, “polypeptide” and “protein” may be used interchangeably, and the term “polypeptide” is often preferred.

Polypeptides may further form multimers such as dimers, trimers and higher oligomers, which consist of more than one polypeptide molecule, as mentioned above. Polypeptide molecules forming such dimers, trimers etc. may be identical or non-identical. The corresponding structures of higher order of such multimers are, consequently, termed homo- or heterodimers, homo- or heterotrimers etc. An example for a hereteromultimer is an antibody or immunoglobulin molecule, which, in its naturally occurring form, consists of two identical light polypeptide chains and two identical heavy polypeptide chains. The terms “peptide”, “polypeptide” and “protein” also refer to naturally modified peptides/polypeptides/proteins wherein the modification is accomplished e.g. by post-translational modifications like glycosylation, acetylation, phosphorylation and the like. A “peptide”, “polypeptide” or “protein” when referred to herein may also be chemically modified such as pegylated. Such modifications are well known in the art and described herein below.

The terms “selectively” and, “preferably, selectively”, “(specifically or immunospecifically) binds to”, “(specifically or immunospecifically) recognizes”, or “(specifically or immunospecifically) reacts with” mean in accordance with this invention that a construct or a binding domain selectively interacts or (immuno-)specifically interacts with a given epitope on the target molecule (antigen), here: CD3, respectively. This selective interaction or association occurs more frequently, more rapidly, with greater duration, with greater affinity, or with some combination of these parameters, to an epitope on the specific target (here: CD3epsilon) than to alternative substances (non-target molecules, e.g., here CD3gamma, etc.). Because of the sequence similarity between homologous proteins in different species, a construct or a binding domain that selectively and/or immunspecifically binds to its target (such as a human target) may, however, cross-react with homologous target molecules from different species (such as, from non-human primates). The terms “selectively binds to”, “specific/immunospecific binding”, etc. can hence include the binding of a construct or binding domain to epitopes or structurally related epitopes in more than one species. In the context of the present invention, a polypeptide of the present invention binds to its respective target structure in a particular manner. Preferably, a polypeptide according to the present invention comprises one paratope per binding domain which specifically or immunospecifically binds to”, “(specifically or immunospecifically) recognizes”, or “(specifically or immunospecifically) reacts with” its respective target structure. This means in accordance with this invention that a polypeptide or a binding domain thereof interacts or (immuno-)specifically interacts with a given epitope on the target molecule (antigen), namely CD3epsilon, and in certain embodiments with a given epitope on at least one further, such as a second and/or a third target molecule. This interaction or association occurs more frequently, more rapidly, with greater duration, with greater affinity, or with some combination of these parameters, to an epitope on the specific target than to alternative substances (non-target molecules). Because of the sequence similarity between homologous proteins in different species, an antibody construct or a binding domain that immunspecifically binds to its target (such as a human target) may, however, cross-react with homologous target molecules from different species (such as, from non-human primates). The term “specific/immunospecific binding” can hence include the binding of an antibody construct or binding domain to epitopes and/or structurally related epitopes in more than one species. The term “(immuno-) selectively binds does exclude the binding to structurally related epitopes.

In the context of the present invention, the term “epitope” refers to the part or region of the antigen that is selectively recognized/immunospecifically recognized by the binding structure, i.e. the paratope. An “epitope” is antigenic, and thus the term epitope is sometimes also referred to as “antigenic structure” or “antigenic determinant”. The part of the binding domain that binds to the epitope is called a paratope. Specific binding is believed to be accomplished by specific motifs in the amino acid sequence of the binding domain and the antigen. Thus, binding is achieved because of their primary, secondary and/or tertiary structure as well as the result of potential secondary modifications of said structures. The specific interaction of the paratope with its antigenic determinant may result in a simple binding of said site to the antigen. In some cases, the specific interaction may alternatively or additionally result in the initiation of a signal, e.g. due to the induction of a change of the conformation of the antigen, an oligomerization of the antigen, etc.

The epitopes of protein antigens are divided into two categories, conformational epitopes and linear epitopes, based on their structure and interaction with the paratope. A conformational epitope is composed of discontinuous sections of the antigen's amino acid sequence. These epitopes interact with the paratope based on the three-dimensional surface features and shape or tertiary structure (folding) of the antigen. Methods of determining the conformation of epitopes include, but are not limited to, x-ray crystallography, two-dimensional nuclear magnetic resonance (2D-NMR) spectroscopy and site-directed spin labelling and electron paramagnetic resonance (EPR) spectroscopy. By contrast, linear epitopes interact with the paratope based on their primary structure. A linear epitope is formed by a continuous sequence of amino acids from the antigen and typically includes at least 3 or at least 4, and more usually, at least 5 or at least 6 or at least 7, for example, about 8 to about 10 amino acids in a unique sequence.

A method for epitope mapping for a given human target protein is described in the following: A pre-defined region (a contiguous amino acid stretch) within said given human target protein is exchanged/replaced with a corresponding region of a target protein paralogue (so long as the binding domain is not cross-reactive with the paralogue used). These human target/paralogue chimeras are expressed on the surface of host cells (such as CHO cells). Binding of the antibody or construct can be tested via FACS analysis. When the binding of the antibody or construct to the chimeric molecule is entirely abolished, or when a significant binding decrease is observed, it can be concluded that the region of human target which was removed from this chimeric molecule is relevant for the immunospecific epitope-paratope recognition. Said decrease in binding is preferably at least 10%, 20%, 30%, 40%, or 50%; more preferably at least 60%, 70%, or 80%, and most preferably 90%, 95% or even 100% in comparison to the binding to human (wild-type) target, whereby binding to the human target is set to be 100%. Alternatively, the above described epitope mapping analysis can be modified by introducing one or several point mutations into the sequence of the human target. These point mutations can e.g. reflect the differences between the human target and its paralogue.

A further method to determine the contribution of a specific residue of a target antigen to the recognition by a construct or binding domain is alanine scanning (see e.g. Morrison KL & Weiss G A. Curr Opin Chem Biol. 2001 June; 5(3):302-7), where each residue to be analyzed is replaced by alanine, e.g. via site-directed mutagenesis. Alanine is used because of its non-bulky, chemically inert, methyl functional group that nevertheless mimics the secondary structure references that many of the other amino acids possess. Sometimes bulky amino acids such as valine or leucine can be used in cases where conservation of the size of mutated residues is desired.

The interaction between the binding domain and the epitope of the target antigen implies that a binding domain exhibits appreciable or significant affinity for the epitope/the target antigen (here: CD3) and, generally, does not exhibit significant affinity for proteins or antigens other than the target antigen—notwithstanding the above discussed cross-reactivity with homologous targets e.g. from other species. “Significant affinity” includes binding with an affinity (dissociation constant, KD) of ≤10⁻⁶ M. Preferably, binding is considered specific when the binding affinity is ≤10⁻⁷ M, ≤10⁻⁸ M, ≤10⁻⁹ M, ≤10⁻¹⁰ M, or even ≤10¹¹ M, or ≤10⁻¹² M. Whether a binding domain (immuno-)specifically reacts with or binds to a target can be tested readily e.g. by comparing the affinity of said binding domain to its desired target protein or antigen with the affinity of said binding domain to non-target proteins or antigens (here: proteins other than CD3). Preferably, a construct of the invention does not significantly bind to proteins or antigens other than CD3 (i.e., the CD3 binding domain does not bind to proteins other than CD3)—unless any further binding domain(s) directed against a further target is/are deliberately introduced into the construct of the invention, in which case the binding of that binding domain to its specific target is also provided by the present invention.

It is envisaged that the affinity of the first domain for human CD3epsilon is ≤100 nM, ≤90 nM, ≤80 nM, ≤70 nM, ≤60 nM, ≤50 nM, ≤40 nM, ≤30 nM or ≤20 nM. These values are preferably measured in a cell-based assay, such as a Scatchard assay. Other methods of determining the affinity are also well-known. These values are preferably measured in a surface plasmon resonance assay, such as a Biacore assay.

The term “does not significantly bind” and “does not selectively bind” mean that a construct or binding domain of the present invention does not bind to a protein or antigen other than said CD3, when said protein or antigen is expressed on the surface of a cell. The construct hence shows reactivity of ≤30%, preferably ≤20%, more preferably ≤10%, particularly preferably ≤9%, ≤8%, ≤7%, ≤6%, ≤5%, ≤4%, ≤3%, ≤2%, or ≤1% with proteins or antigens other than CD3 (when said proteins or antigens are expressed on the surface of a cell), whereby binding to CD3, respectively, is set to be 100%. The “reactivity” can e.g. be expressed in an affinity value (see above).

It is envisaged that the construct of the invention (and more specifically the domain comprising a paratope/binding domain that binds to human CD3epsilon) does not bind or does not significantly bind to CD3epsilon paralogues, more specifically to human CD3epsilon paralogues and/or to macaque/cyno CD3epsilon paralogues. It is also envisaged that the construct does not bind or does not significantly bind to (human or macaque/cyno) CD3epsilon paralogues on the surface of a target cell.

The VH region as part of the binding domain binding to an extracellular epitope of the human CD3ε chain comprises a CDR-H1, a CDR-H2 and CDR-H3 sequence as set out herein above. The CDR sequences contain placeholders denoted as “X” for a given amino acid residue that can take the place of the placeholder as is common practice in the art. The placeholders are consecutively numbered in the format X₁, X₂, X₃, and so forth, per given CDR sequence. The amino acid residues that can take place of the placeholder in the recited CDR sequences are listed according to preference, wherein the first listed amino acid is the most preferred, the second listed is the second most preferred, and so forth, if not otherwise specified, e.g. in other embodiments. If a sequence contains more than one placeholder, the same applies in relation to the preference of the amino acids; namely, it is the combination of the first listed amino acid residue for each of the placeholders that is most preferred, the combination of the second listed amino acid residue for each of the placeholders that is second most preferred, and so forth. This hierarchy of preference does not exclude the combination of amino acid residues for each of the placeholders independent of said preference hierarchy so that amino acid residues of different preference are combined. The same hierarchy is true for all amino acid residues that are listed herein below to take the place of a place holder in an amino acid sequence described herein below. Accordingly, the above also applies for the placeholders denoted as “X” in the sequences of the VH and VL sequences and any other sequences recited throughout the specification.

The CDR-H1 sequence comprises the amino acid sequence of X₁YAX₂N, where X, is K, V, S, G, R, T, or I; and X₂ is M or I. Following the above-explained hierarchy as regards the placeholder amino acids, the amino acids for the placeholders X₁ and X₂ are listed according to their preference, wherein the first listed amino acid is the most preferred, the second listed is the second-most preferred, and so forth. For X₁, the amino acid K is most preferred, and for X₂, the amino acid M is most preferred. A preferred combination is M for X₂ in combination with any of the amino acid residues of X₁, such as the combinations for X₁ and X₂, respectively, selected from K and M, V and M, S and M, most preferred the combination K and M. Another preferred combination is K and I, for X₁ and X₂, respectively. Preferred examples of the CDR-H1 sequence are selected from KYAMN, VYAMN, SYAMN, GYAMN, RYAMN, TYAMN, IYAMN, and KYAIN, wherein KYAMN or KYAIN is most preferred.

The CDR-H2 sequence comprises the amino acid sequence of RIRSKYNNYATYYADX₁VK X₂, where X, is S or Q; and X₂ is D, G, K, S, or E. For X₁, the amino acid S is most preferred, and for X₂, the amino acid D is most preferred. A preferred combination is S for X₁ in combination with any of the amino acid residues of X₂, such as the combinations for X₁ and X₂, respectively, selected from S and D, S and G, S and K, S and S, most preferred the combination S and D. Preferred examples of the CDR-H2 sequence are selected from RIRSKYNNYATYYADSVKD, RIRSKYNNYATYYADSVKK, RIRSKYNNYATYYADSVKS, RIRSKYNNYATYYADSVKE, RIRSKYNNYATYYADSVKG, and RIRSKYNNYATYYADQVKD, wherein RIRSKYNNYATYYADSVKD is most preferred.

The CDR-H3 sequence comprises the amino acid sequence of HX₁NFGNSYX₂SX₃X₄AY, where X, is G, R, or A; X₂ is I, L, V, or T; X₃ is Y, W or F; and X₄ is W, F or Y. For X₁, the amino acid G is most preferred; for X₂, the amino acid I is most preferred; for X₃, the amino acid Y is most preferred; and for X₄, the amino acid W is most preferred. Preferred examples of the CDR-H3 sequence are selected from HGNFGNSYISYWAY, HGNFGNSYLSWWAY, HGNFGNSYTSYYAY, HRNFGNSYLSWFAY, HGNFGNSYVSFFAY, HGNFGNSYISWWAY, HGNFGNSYVSWWAY, HGNFGNSYLSYFAY, HGNFGNSYLSFWAY, HANFGNSYISYWAY, and HGNFGNSYVSWFAY, wherein HGNFGNSYISYWAY is most preferred.

The CDR-L1 sequence comprises the amino acid sequence of X₁SSTGAVTX₂X₃X₄YX₅N, where X, is G, R, or A; X₂ is S or T; X₃ is G or S; X₄ is N or Y; and X₅ is P or A. For X₁, the amino acid G is most preferred; for X₂, the amino acid S is most preferred; for X₃, the amino acid G is most preferred; for X₄, the amino acid N is most preferred; and for X₅, the amino acid P is most preferred. Preferred examples of the CDR-L1 sequence are selected from GSSTGAVTSGNYPN, RSSTGAVTSGYYPN, GSSTGAVTSGYYPN, ASSTGAVTSGNYPN, and RSSTGAVTTSNYAN, wherein GSSTGAVTSGNYPN is most preferred.

The CDR-L2 sequence comprises the amino acid sequence of X₁TX₂X₃X₄X₅X₆; where X, is G or A; X₂ is K, D, or N; X₃ is F, M or K; X₄ is L or R; X₅ is A, P, or V; and X₆ is P or S. For X₁, the amino acid G is most preferred; for X₂, the amino acid K is most preferred; for X₃, the amino acid F is most preferred; for X₄, the amino acid L is most preferred; for X₅, the amino acid A is most preferred and for X₆, the amino acid P is most preferred. Preferred examples of the CDR-L2 sequence are selected from GTKFLAP, ATDMRPS, GTKFLVP, and GTNKRAP, wherein GTKFLAP is most preferred.

The CDR-L3 sequence comprises the amino acid sequence of X₁LWYSNX₂WV, where X, is V, A, or T; and X₂ is R or L. For X₁, the amino acid V is most preferred, and for X₂, the amino acid R is most preferred. Preferred combinations are R for X₂ in combination with any of the amino acid residues of X₁, such as the combinations for X₁ and X₂, respectively, selected from V and R, A and R, T and R, most preferred the combination V and R. Preferred examples of the CDR-L3 sequence are selected from VLWYSNRWV, ALWYSNRWV, TLWYSNRWV, and ALWYSNLWV, wherein VLWYSNRWV is most preferred.

In a preferred embodiment, said VH region of i) comprises: a CDR-H1 sequence comprising or consisting of the amino acid sequence of X₁YAX₂N, where X, is K, V, S, G, R, or I; and X₂ is M or I; a CDR-H2 sequence of RIRSKYNNYATYYADX₁VKX₂, where X, is S or Q; and X₂ is D, G, K, S, or E; and a CDR-H3 sequence of HX₁NFGNSYX₂SX₃X₄AY, where X, is G, R, or A; X₂ is I, L, V, or T; X₃ is Y, W or F; and X₄ is W, F or Y; and said VL region of ii) comprises: a CDR-L1 sequence of X₁SSTGAVTSGX₂YPN, where X, is G, R, or A; and X₂ is N or Y; a CDR-L2 sequence of X₁TX₂X₃X₄X₅X₆; where X, is G or A; X₂ is K or D; X₃ is F or M; X₄ is L or R; X₅ is A, P, or V; and X₆ is P or S; and a CDR-L3 sequence of X₁LWYSNRWV, where X, is V, A, or T; and wherein said one or more CDR sequences of the VH region of i) and/or of the VL region of ii) comprise one or more amino acid substitutions selected from X₂4V or X₂4F in CDR-H1; D15 (preferably E), X₁16A in CDR-H2; H1 (preferably A or N), X₁2E, F4 (preferably I), and/or N6 (preferably S or T) in CDR-H3; and W93 (preferably Y) in CDR-L3.

Said CDR-H1 sequence comprises or consists of the amino acid sequence of X₁YAX₂N, where X, is K, V, S, G, R, or I; and X₂ is M or I. For X₁, the amino acid K is most preferred, and for X₂, the amino acid M is most preferred. A preferred combination is M for X₂ in combination with any of the amino acid residues of X₁, such as the combinations for X₁ and X₂, respectively, selected from K and M, V and M, S and M, most preferred the combination K and M. Another preferred combination is K and I, for X₁ and X₂, respectively. Preferred examples of the CDR-H1 sequence are selected from KYAMN, VYAMN, SYAMN, GYAMN, RYAMN, IYAMN, and KYAIN, wherein KYAMN or KYAIN is preferred, wherein KYAMN is most preferred.

Said CDR-H2 sequence comprises or consist of the amino acid sequence of RIRSKYNNYATYYADX₁VK X₂, where X, is S or Q; and X₂ is D, G, K, S, or E. For X₁, the amino acid S is most preferred, and for X₂, the amino acid D is most preferred. A preferred combination is S for X₁ in combination with any of the amino acid residues of X₂, such as the combinations for X₁ and X₂, respectively, selected from S and D, S and G, S and K, S and S, most preferred the combination S and D. Preferred examples of the CDR-H2 sequence are selected from RIRSKYNNYATYYADSVKD, RIRSKYNNYATYYADSVKK, RIRSKYNNYATYYADSVKS, RIRSKYNNYATYYADSVKE, RIRSKYNNYATYYADSVKG, and RIRSKYNNYATYYADQVKD, wherein RIRSKYNNYATYYADSVKD is most preferred.

The CDR-H3 sequence comprises the amino acid sequence of HX₁NFGNSYX₂SX₃X₄AY, where X, is G, R, or A; X₂ is I, L, V, or T; X₃ is Y, W or F; and X₄ is W, F or Y. For X₁, the amino acid G is most preferred; for X₂, the amino acid I is most preferred; for X₃, the amino acid Y is most preferred; and for X₄, the amino acid W is most preferred. Preferred examples of the CDR-H3 sequence are selected from HGNFGNSYISYWAY, HGNFGNSYLSWWAY, HGNFGNSYTSYYAY, HRNFGNSYLSWFAY, HGNFGNSYVSFFAY, HGNFGNSYISWWAY, HGNFGNSYVSWWAY, HGNFGNSYLSYFAY, HGNFGNSYLSFWAY, HANFGNSYISYWAY, and HGNFGNSYVSWFAY, wherein HGNFGNSYISYWAY is most preferred.

The CDR-L1 sequence comprises the amino acid sequence of X₁SSTGAVTSGX₂YPN, where X, is G, R, or A; and X₂ is N or Y. For X₁, the amino acid G is most preferred; and for X₂, the amino acid N is preferred. Preferred examples of the CDR-L1 sequence are selected from GSSTGAVTSGNYPN, RSSTGAVTSGYYPN, GSSTGAVTSGYYPN, and ASSTGAVTSGNYPN, wherein GSSTGAVTSGNYPN or ASSTGAVTSGNYPN is preferred, wherein GSSTGAVTSGNYPN is most preferred.

The CDR-L2 sequence comprises the amino acid sequence of X₁TX₂X₃X₄X₅X₆; where X, is G or A; X₂ is K or D; X₃ is F or M; X₄ is L or R; X₅ is A, P, or V; and X₆ is P or S. For X₁, the amino acid G is most preferred; for X₂, the amino acid K is most preferred; for X₃, the amino acid F is most preferred; for X₄, the amino acid L is most preferred; for X₅, the amino acid A is most preferred and for X₆, the amino acid P is most preferred. Preferred examples of the CDR-L2 sequence are selected from GTKFLAP, ATDMRPS, and GTKFLVP, wherein GTKFLAP or GTKFLVP is preferred, wherein GTKFLAP is most preferred.

The CDR-L3 sequence comprises the amino acid sequence of X₁LWYSNRWV, where X, is V, A, or T, wherein the amino acid V is most preferred. Preferred examples of the CDR-L3 sequence are selected from VLWYSNRWV, ALWYSNRWV, and TLWYSNRWV, wherein VLWYSNRWV or TLWYSNRWV is more preferred, wherein VLWYSNRWV is most preferred.

In a further preferred embodiment, said VH region of i) comprises: a CDR-H1 sequence comprising or consisting of the amino acid sequence of X₁YAX₂N, where X, is K, V, S, R, or I; and X₂ is M or I; a CDR-H2 sequence of RIRSKYNNYATYYADX₁VKX₂, where X, is S or Q; and X₂ is D, G, K, or S; and a CDR-H3 sequence of HX₁NFGNSYX₂SX₃X₄AY, where X, is G or A; X₂ is I, L, V, or T; X₃ is Y, W or F; and X₄ is W, F or Y; and said VL region of ii) comprises: a CDR-L1 sequence of X₁SSTGAVTSGX₂YPN, where X, is G or A; and X₂ is N or Y; a CDR-L2 sequence of GTKFLX₁P; where X, is A or V; and a CDR-L3 sequence of X₁LWYSNRWV, where X, is V, A, or T; and wherein said one or more CDR sequences of the VH region of i) and/or of the VL region of ii) comprise one or more amino acid substitutions selected from X₂4V or X₂4F in CDR-H1; D15 (preferably E), X₁16A in CDR-H2; H1 (preferably A or N), X₁2E, F4 (preferably I), and/or N6 (preferably S or T) in CDR-H3; and W93 (preferably Y) in CDR-L3.

Said CDR-H1 sequence comprises or consists of the amino acid sequence of X₁YAX₂N, where X, is K, V, S, R, or I; and X₂ is M or I. For X₁, the amino acid K is most preferred, and for X₂, the amino acid M is most preferred. A preferred combination is M for X₂ in combination with any of the amino acid residues of X₁, such as the combinations for X₁ and X₂, respectively, selected from K and M, V and M, S and M, most preferred the combination K and M. Another preferred combination is K and I, for X₁ and X₂, respectively. Preferred examples of the CDR-H1 sequence are selected from KYAMN, VYAMN, SYAMN, RYAMN, IYAMN, and KYAIN, wherein KYAMN or KYAIN is preferred, wherein KYAMN is most preferred.

Said CDR-H2 sequence comprises or consist of the amino acid sequence of RIRSKYNNYATYYADX₁VKX₂, where X, is S or Q; and X₂ is D, G, K, or S. For X₁, the amino acid S is most preferred, and for X₂, the amino acid D is most preferred. A preferred combination is S for X₁ in combination with any of the amino acid residues of X₂, such as the combinations for X₁ and X₂, respectively, selected from S and D, S and G, S and K, S and S, most preferred the combination S and D. Preferred examples of the CDR-H2 sequence are selected from RIRSKYNNYATYYADSVKD, RIRSKYNNYATYYADSVKK, RIRSKYNNYATYYADSVKS, RIRSKYNNYATYYADSVKG, and RIRSKYNNYATYYADQVKD, wherein RIRSKYNNYATYYADSVKD or RIRSKYNNYATYYADQVKD is preferred, wherein RIRSKYNNYATYYADSVKD is most preferred.

Said CDR-H3 sequence comprises or consist of the amino acid sequence of HX₁NFGNSYX₂SX₃X₄AY, where X, is G or A; X₂ is I, L, V, or T; X₃ is Y, W or F; and X₄ is W, F or Y. For X₁, the amino acid G is most preferred; for X₂, the amino acid I is most preferred; for X₃, the amino acid Y is most preferred; and for X₄, the amino acid W is most preferred. Preferred examples of the CDR-H3 sequence are selected from HGNFGNSYISYWAY, HGNFGNSYLSWWAY, HGNFGNSYTSYYAY, HGNFGNSYVSFFAY, HGNFGNSYISWWAY, HGNFGNSYVSWWAY, HGNFGNSYLSYFAY, HGNFGNSYLSFWAY, and HANFGNSYISYWAY, wherein HGNFGNSYISYWAY or HANFGNSYISYWAY is preferred, wherein HGNFGNSYISYWAY is most preferred.

Said CDR-L1 sequence comprises or consists the amino acid sequence of X₁SSTGAVTSGX₂YPN, where X, is G or A; and X₂ is N or Y. For X₁, the amino acid G is most preferred; and for X₂, the amino acid N is preferred. Preferred examples of the CDR-L1 sequence are selected from GSSTGAVTSGNYPN, GSSTGAVTSGYYPN, and ASSTGAVTSGNYPN, wherein GSSTGAVTSGNYPN or ASSTGAVTSGNYPN is preferred, wherein GSSTGAVTSGNYPN is most preferred.

Said CDR-L2 sequence comprises or consists of the amino acid sequence of GTKFLX₁P; where X, is A or V. For X₁, the amino acid A is preferred. Said CDR-L2 sequence is GTKFLAP or GTKFLVP, wherein GTKFLAP is preferred.

The CDR-L3 sequence comprises or consists of the amino acid sequence of X₁LWYSNRWV, where X, is V, A, or T, wherein the amino acid V is most preferred. Preferred examples of the CDR-L3 sequence are selected from VLWYSNRWV, ALWYSNRWV, and TLWYSNRWV, wherein VLWYSNRWV or TLWYSNRWV is more preferred, wherein VLWYSNRWV is most preferred.

In an even more preferred embodiment, said VH region of i) comprises: a CDR-H1 sequence comprising or consisting of the amino acid sequence of X₁YAMN, where X, is K or S; a CDR-H2 sequence of RIRSKYNNYATYYADSVKX₁, where X, is D or G; and a CDR-H3 sequence of HGNFGNSYX₁SX₂WAY, where X, is I or V; and X₂ is Y or W; and said VL region of ii) comprises: a CDR-L1 sequence of GSSTGAVTSGX₁YPN, where X, is N or Y; a CDR-L2 sequence of GTKFLAP; and a CDR-L3 sequence of X₁LWYSNRWV, where X, is V or A; and wherein said one or more CDR sequences of the VH region of i) and/or of the VL region of ii) comprise one or more amino acid substitutions selected from M4V or M4F in CDR-H1; D15 (preferably E), S16A in CDR-H2; H1 (preferably A or N), G2E, F4 (preferably I), and/or N6 (preferably S or T) in CDR-H3; and W93 (preferably Y) in CDR-L3.

Said CDR-H1 sequence comprises or consists of the amino acid sequence of X₁YAMN, where X, is K or S, wherein the amino acid K is preferred. Said CDR-H1 sequence is KYAMN or SYAMN, wherein KYAMN is most preferred.

Said CDR-H2 sequence comprises or consist of the amino acid sequence of RIRSKYNNYATYYADSVKX₁, where X, is D or G. For X₁, the amino acid D is preferred. Said CDR-H2 sequence is RIRSKYNNYATYYADSVKD or RIRSKYNNYATYYADSVKG, wherein RIRSKYNNYATYYADSVKD is preferred.

Said CDR-H3 sequence comprises or consist of the amino acid sequence of HGNFGNSYX₁SX₂WAY, where X, is I or V; and X₂ is Y or W. For X₁, the amino acid I is preferred; for X₂, the amino acid Y is preferred. Said CDR-H3 sequence is HGNFGNSYISYWAY or HGNFGNSYVSWWAY, wherein HGNFGNSYISYWAY is preferred.

Said CDR-L1 sequence comprises or consists the amino acid sequence of GSSTGAVTSGX₁YPN, where X, is N or Y. For X₁, the amino acid N is preferred. Said CDR-L1 sequence is GSSTGAVTSGNYPN or GSSTGAVTSGYYPN, wherein GSSTGAVTSGNYPN is preferred.

Said CDR-L2 sequence comprises or consists of the amino acid sequence of GTKFLAP.

The CDR-L3 sequence comprises or consists of the amino acid sequence of X₁LWYSNRWV, where X, is V or A, wherein V is preferred. Said CDR-L3 sequence VLWYSNRWV or ALWYSNRWV, wherein VLWYSNRWV is preferred.

Each of the CDR-H1, CDR-H2, CDR-H3, CDR-L1, CDR-L2 and CDR-L3 sequences be freely combined in the format CDR-H1, CDR-H2, CDR-H3, CDR-L1, CDR-L2, CDR-L3 (this is the preferred orientation of the VH and the VL region) or CDR-L1, CDR-L2, CDR-L3, CDR-H1, CDR-H2, CDR-H3 and exhibiting the recited binding to an extracellular epitope of the human CD3ε chain.

Preferred combinations of CDR-L1 to L3 sequences of the VL region and preferred combinations of CDR-H1 to H3 sequences of the VH region are listed in the below Table 1.

TABLE 1

| CDR-L1 | CDR-L2 | CDR-L3 |
|---|---|---|
| GSSTGAVTSGYYP | GTKFLAP | ALWYSNRWV |
| N | | |
| RSSTGAVTSGYYP | ATDMRPS | ALWYSNRWV |
| N | | |
| GSSTGAVTSGNYP | GTKFLAP | VLWYSNRWV |
| N | | |
| ASSTGAVTSGNYP | GTKFLVP | TLWYSNRWV |
| N | | |
| RSSTGAVTTSNYA | GTNKRAP | ALWYSNLWV |
| N | | |
| CDR-H1 | CDR-H2 | CDR-H3 |
| IYAMN | RIRSKYNNYATYYADSVKS | HGNFGNSYVSFFAY |
| KYAMN | RIRSKYNNYATYYADSVKD | HGNFGNSYISYWAY |
| SYAMN | RIRSKYNNYATYYADSVKG | HGNFGNSYLSFWAY |
| RYAMN | RIRSKYNNYATYYADSVKG | HGNFGNSYLSYFAY |
| VYAMN | RIRSKYNNYATYYADSVKK | HGNFGNSYLSWWAY |
| KYAMN | RIRSKYNNYATYYADSVKS | HGNFGNSYTSYYAY |
| GYAMN | RIRSKYNNYATYYADSVKE | HRNFGNSYLSWFAY |
| VYAMN | RIRSKYNNYATYYADSVKK | HGNFGNSYISWWAY |
| SYAMN | RIRSKYNNYATYYADSVKG | HGNFGNSYVSWWAY |
| KYAIN | RIRSKYNNYATYYADQVKD | HANFGNSYISYWAY |
| TYAMN | RIRSKYNNYATYYADSVKD | HGNFGNSYVSWFAY |

Preferably, any of the above listed combinations of CDR-L1 to L3 combinations is combined with any of the above-listed combinations CDR-H1 to H3 as part of the binding domain binding to an extracellular of the human CD3ε chain. In other words, the VL region comprises or consists of as CDR-L1, CDR-L2, CDR-L3 sequence, in this order,

GSSTGAVTSGYYPN, GTKFLAP, ALWYSNRWV;
RSSTGAVTSGYYPN, ATDMRPS, ALWYSNRWV;
GSSTGAVTSGNYPN, GTKFLAP, VLWYSNRWV;
ASSTGAVTSGNYPN, GTKFLVP, TLWYSNRWV;
or
RSSTGAVTTSNYAN, GTNKRAP, ALWYSNLWV;

the VL region comprises or consists of as CDR-H1, CDR-H2, CDR-H3 sequence, in this order,

IYAMN, RIRSKYNNYATYYADSVKS, HGNFGNSYVSFFAY;
KYAMN, RIRSKYNNYATYYADSVKD, HGNFGNSYISYWAY;
SYAMN, RIRSKYNNYATYYADSVKG, HGNFGNSYLSFWAY;
RYAMN, RIRSKYNNYATYYADSVKG, HGNFGNSYLSYFAY;
VYAMN, RIRSKYNNYATYYADSVKK, HGNFGNSYLSWWAY;
KYAMN, RIRSKYNNYATYYADSVKS, HGNFGNSYTSYYAY;
GYAMN, RIRSKYNNYATYYADSVKE, HRNFGNSYLSWFAY;
VYAMN, RIRSKYNNYATYYADSVKK, HGNFGNSYISWWAY;
SYAMN, RIRSKYNNYATYYADSVKG, HGNFGNSYVSWWAY;
KYAIN, RIRSKYNNYATYYADQVKD, HANFGNSYISYWAY;
or
TYAMN, RIRSKYNNYATYYADSVKD, HGNFGNSYVSWFAY.

In accordance with the invention, preferred CDR sequence combinations of the VH and the VL regions, listed in the order CDR-H1, CDR-H2, CDR-H3, CDR-L1, CDR-L2, CDR-L3, are as defined in SEQ ID NOs: 118 to 123, 166 to 171, 356 to 361, 546 to 551, 724 to 729, 922 to 927, 1100 to 1105, 1290 to 1295, 1468 to 1473, 1658 to 1663, 1848 to 1853.
Most preferred is that the VL region comprises as CDR-L1, CDR-L2, CDR-L3 sequence, listed in this order,

### GSSTGAVTSGNYPN, GTKFLAP, VLWYSNRWV; and

the VL region comprises as CDR-H1, CDR-H2, CDR-H3 sequence, listed in this order, KYAMN, RIRSKYNNYATYYADSVKD, HGNFGNSYISYWAY; as depicted in SEQ ID NOs: 1848 to 1853. Preferably the orientation of the CDRs is VH to VL, i.e. the orientation of the variable regions is from N- to C-terminus VH to VL.

In accordance with the invention, said CDR sequences of the VH and/or the VL region comprise one or more amino acid substitutions. As is common in the art, the position and nature of amino acid substitutions are denoted herein by indicating the original amino acid residue at the position to be substituted and the amino acid used for substitution in the format of, e.g., F41, wherein “F” denotes the original amino acid residue occurring at position “4” of the given amino acid sequence and “I” denotes the amino acid residue which substituted “F” at position 4. In case substitution is by any amino acid residue (other than the original amino acid residue), no amino acid residue is indicated, but only the originally occurring amino acid residue and its position in the given amino acid sequence, so that the previous example would be denoted as “F4” only. Preferably, only naturally occurring amino acids are used for substitution.

The VH region of i) and/or VL region of ii) comprise one or more amino acid substitutions as specified herein in one or more CDR sequences as recited herein above. Accordingly, at least one of the recited amino acid substitutions of the variable regions is comprised in said binding domain binding to an extracellular epitope of the human CD3ε chain. Said at least one or one or more amino acid substitution, in the case that only one amino acid substation is present, can be present in a CDR sequence in said VH or said VL region or, in case more than one amino acid substitution is present, in CDRs in both the VH and the VL region or restricted to CDR sequences in only the VH or the VL region. In certain embodiments where there are more than one amino acid substitutions, the substitutions are in the VH and the VL region or, alternatively, only in one variable region. Preferably, the substitutions are in the CDRs of the VH and the VL region.

In case the CDR-H1 is to comprise an amino acid substitution at position 4 it is, preferably, the substitution of the original amino acid with the amino acid V. Preferably, the CDR-H1 sequence is KYAIN or KYAMN, i.e. comprising none of said recited amino acid substitutions.

In CDR-H2, the amino acid substitution at position 15 is, preferably, D15E, or the amino acid substitution at position 16 with amino acid A. Preferably, CDR-H1 comprises the amino acid residue substitution at position 16 with amino acid A; in this case, a preferred amino acid for the last amino acid of CDR-H2 is D or G, most preferred D. Hence, a preferred CDR-H2 sequence is RIRSKYNNYATYYADAVKD or RIRSKYNNYATYYADAVKG, with RIRSKYNNYATYYADAVKD more preferred. In case CDR-H2 contains two amino acid substitutions, the combination of the amino acid E at position 15 and amino acid A at position 16 are preferred; in this case, a preferred amino acid for the last amino acid of CDR-H2 is G. Hence, a preferred CDR-H2 sequence for the combination of the amino acid E at position 15 and amino acid A at position 16 is RIRSKYNNYATYYAEAVKG.

In CDR-H3, the amino acid substitutions are at position 1, preferably a substitution with amino acid A or N, at position 2 with amino acid E, at position 4, preferably with amino acid I, and at position 6, preferably with amino acid S or T. Preferably, CDR-H3 comprises the amino acid A or N at position 1, the amino acid S or T at position 6; in this case and if placeholders are present in the CDR-H3 sequence at the indicated positions, preferred amino acids for X₁ are G or A, most preferred G; for X₂ is I; for X₃ is Y; and for X₄ are W or Y. In case CDR-H3 comprises two amino acid substitutions, preferred combinations are the amino acid A at position 1 and the amino acid S at position 6; and the amino acid N at position 1 and the amino acid T at position 6; in this case and if placeholders are present in the CDR-H3 sequence at the indicated positions, a preferred amino acid for X₁ is G or A, most preferred G; for X₂ is I, for X₃ is Y, and for X₄ are W and F. In case CDR-H3 comprises three amino acid substitutions, preferred combinations are the amino acid A at position 1, the amino acid I at position 4, and the amino acid S at position 6; and the amino acid N at position 1, the amino acid I at position 4 and the amino acid T at position 6, in this case and if placeholders are present in the CDR-H3 sequence at the indicated positions, a preferred amino acid for X₂ is I, for X₃ is Y, and for X₄ are W and F. In case CDR-H3 comprises four amino acid substitutions, a preferred combination is the amino acid N at position 1, the amino acid E at position 2, the amino acid I at position 4, and the amino acid T at position 6; in this case and if placeholders are present in the CDR-H3 sequence at the indicated positions, a preferred amino acid for X₂ is I, for X₃ is Y, and for X₄ is W. Hence, preferred CDR-H3 sequences are selected from AGNFGSSYISYWAY, NENIGTSYISYWAY, AGNFGTSYISYWAY, NANFGTSYISYFAY, and AGNFGSSYISYFAY, with AGNFGSSYISYWAY and AGNFGSSYISYFAY most preferred.

In CDR-L3, the amino acid substitution at position 1 is with amino acid L or the amino acid substitution is W93, preferably W93Y (which may also be referred to as X₁93Y), in the sequence of X₁LWYSNX₂WV, where X, is V, A, or T; and X₂ is R or L. Preferably, the CDR-L3 sequence is VLYYSNRWV when CDR-L3 comprises only one amino acid substitution. It is preferred, however, that CDR-L3 does not comprise said X₁1L and W93 amino acid substitution, in which case a preferred CDR-L3 sequence is VLYYSNRWV.

Without wishing to be bound by a specific scientific theory, each of the above recited amino acid substitution(s) lead(s) to an increase in thermal stability of the binding domain binding to an extracellular epitope of the human CD3ε chain comprising or consisting of a VH region linked to a VL region as described herein as compared to the binding domain binding to an extracellular epitope of the human CD3ε chain comprising or consisting of a VH region linked to a VL region sequence that is unmodified, i.e. does not contain said amino acid residue substitution(s). Said increase of the temperature stability is, preferably, measured by the well know and also herein described method of Differential Scanning Fluorimetry (DSF). Said method is, e.g., described in Wen et al., “Nano differential scanning fluorimetry for comparability studies of therapeutic proteins”, Analytical Biochemistry, Volume 593, 2020, 113581, ISSN 0003-2697; or Dart, M. L., et al. (2018). “Homogeneous Assay for Target Engagement Utilizing Bioluminescent Thermal Shift”. ACS medicinal chemistry letters, 9(6), 546-551). As evident from the example section, it was shown that the amino acid substitutions or combinations thereof as defined herein result in an increased thermal stability (see Example 1, Table 2 and 3) as compared to the unmodified CD3 binding domain defined herein (“I2C”) while the cytotoxic activity was maintained which was not the case with a prior art method to improve thermal stability, namely the CC44/100 cys clamp, in which an additional disulfide bond was engineered for stabilizing (Reiter, Y. et al., 1994) by replacing the amino acid residues with cysteine residues at position 44 in the heavy chain variable region and at position 100 in the light chain variable region (numbering scheme according to Kabat et al., 1991) of the unmodified CD3 binding domain defined herein (VH and VL SEQ ID NOs: 1854 and 1855, respectively; see Example 2, Table 5). The VH and VL region combinations of the CD3epsilon binding domain described herein are therefore temperature stabilized as compared to the VH and VL region sequence that does not contain the given substitution(s) that are introduced at the positions defined herein.

In a preferred embodiment of the polypeptide or polypeptide construct of claim 1 of the invention, in addition to said one amino acid substitution or a combination thereof as defined in iii) X₂ is I in said CDR-H1 sequence; X₂ is G in said CDR-H2 sequence; X₁ is A, X₄ is F in said CDR-H3 sequence; and/or X₁ is A in said CDR-L3 sequence. The one amino acid substitution or combination thereof as defined herein is/are in addition combined with specific amino acids at specific positions in the CDRs. Hence, at least one of the recited amino acid substitutions in accordance with iii) is present and at least one of X₂ is G in said CDR-H2 sequence; X₁ is A, X₄ is F in said CDR-H3; and/or X₁ is A in said CDR-L3 sequence is additionally present in the CDRs. As previously stated herein above, a preferred amino acid for X₂ is I in said CDR-H1 sequence or X₂ is G in said CDR-H2 sequence, if only one additional amino acid is present in the CDRs, wherein it is most preferred that X₂ is I in said CDR-H1 sequence. A preferred combination of two of said additional amino acids is X₂ is I in said CDR-H1 sequence and X₄ is F in said CDR-H3 sequence; or X₂ is G in said CDR-H2 sequence and X₄ is F in said CDR-H3 sequence. A preferred combination of three of said additional amino acids is X₂ is I in said CDR-H1 sequence, X, is A, and X₄ is F in said CDR-H3 sequence; or X₂ is I in said CDR-H1 sequence, X₄ is F in said CDR-H3 sequence, and X, is A in said CDR-L3 sequence. Since the latter amino acids are to be present additionally, it is understood that those recited additional amino acids that overlap in position with the amino acid substitution of iii) cannot replace the overlapping amino acid substitution. For example, if the only amino acid substitution in accordance with iii) is either X₂4V or X₂4F in CDR-H1, said amino acid cannot be replaced by amino acid I in said CDR-H1 sequence, since there would be no amino acid substitution as defined in iii) remaining in the binding domain. In other terms, there must always be at least one of the amino acid substitutions as defined in iii) comprised in the polypeptide or polypeptide construct of the invention.

It is also understood that depending on the actual sequence of the CDRs, one or more of said additional amino acids may already be part of the CDRs as defined herein above or, if this is not the case, those CDRs will be changed to exhibit said additional amino acid(s) in combination with one amino acid substitution or a combination thereof as recited in iii).

In another preferred embodiment, the polypeptide or polypeptide construct of the invention comprises or consists of combinations of said amino acid substitutions as defined in iii) of two, three, four, five, six, seven, eight, nine, ten, eleven, twelve or more amino acid substitutions, or of up to two, three, four, five, six, seven, eight, nine, ten, eleven, twelve amino acid substitutions. Preferably, only the substitution(s) defined herein are introduced in the VH and the VL region sequence. This applies to all embodiments recited herein so that the VH and the VL region sequence consists of the unmodified amino acid sequence with the substitution(s) as defined herein.

More specifically, it is preferred that i) said one amino acid substitution is selected from: a. X₂4V or X₂4F in CDR-H1; H1A in CDR-H3; and b. X₁1L and W3Y in CDR-L3; ii) said combination of two or more amino acid substitutions are selected from X₁16A in CDR-H2 and N6S in CDR-H3; and X₁16A in CDR-H2 and N6T in CDR-H3; iii) said combination of three or more amino acid substitutions are selected from X₁16A in CDR-H2, H1A and N6S in CDR-H3; X₁16A in CDR-H2, H1A and N6T in CDR-H3; X₁16A in CDR-H2, H1N and N6T in CDR-H3; iv) said combination of four or more amino acid substitutions are selected from X₁16A in CDR-H2, H1A, N6S in CDR-H3, and W3Y in CDR-L3; D15E, X₁16A in CDR-H2, H1A, and N6T in CDR-H3; D15E, X₁16A in CDR-H2, H1A, and N6S in CDR-H3; X₁16A in CDR-H2, H1A, F41 and N6S in CDR-H3; v) said combination of five or six amino acid substitutions are selected from X₁16A in CDR-H2, H1A, X₁2E, F41, and N6T in CDR-H3; X₁16A in CDR-H2, H1N, X₁2E, F41, and N6S in CDR-H3; D15E, X₁16A in CDR-H2, H1A, N6S in CDR-H3; and W3Y in CDR-L3; D15E, X₁16A in CDR-H2, H1N, X₁2E, F41 and N6T in CDR-H3; D15E, X₁16A in CDR-H2, H1A, X₁2E, F41 and N6T in CDR-H3; D15E, X₁16A in CDR-H2, X₁2E, F41 and N6T in CDR-H3.

In accordance with the foregoing, the amino acid substitution H1A in CDR-H3 is preferably the amino acid substitution, in case there is only one amino acid substitution in the CD3epsilon binding domain, within the CDR sequences or the VH region sequences as defined herein. Furthermore, in case of combinations of amino acid substitutions as defined in iii) preferably H1A in CDR-H3 is one of the amino acid substitutions of said combination.

In an embodiment, the invention relates to a polypeptide or polypeptide construct comprising: a binding domain binding to an extracellular epitope of the human CD3ε chain comprising or consisting of a VH region and a VL region, wherein
i) the VH region comprises the sequence of EVX₁LX₂ESGGGLX₃QPX₄GSLKLSCAASGFTFNX₅YAX₆NWVRQAPGKGLEWVA RIRSKYNNYATYYADX₇VKX₃RFTISRDDSXX₁₀X₁₁X₁₂YLQMNNLKTEDTAX₁₃YYC VRHX₁₄NFGNSYX₁₅SX₁₆X₁₇AYWGQGTLVTVSX₁₈, where X₁ is Q or K; X₂ is V or L; X₃ is V or E; X₄ is G or K; X₅ is K, V, S, G, R, T, or I; X₆ is M or I; X₇ is S or Q; X₈ is D, G, K, S, or E; X₉ is K or Q; X₁₀ is N or S; X₁₁ is T or 1; X₁₂ is A or L; X₁₃ is V or M; X₁₄ is G, R or A; X₁₅ is I, L, V, or T; X₁₆ is Y, W or F; X₁₇ is W, F or Y; and X₁₈ is S or A; and
ii) the VL region comprises the sequence of QX₁VVTQEX₂X₃LTX₄SPGX₅TVTLTCX₆SSTGAVTX₇X₈X₉YX₁₀NWVQX₁ KPX₁₂X₁₃ X₁₄X₁₅X₁₆GLIGX₁₇TX₁₈X₁₉X₂₀X₂₁X₂₂GX₂₃PARFSGSLX₂₄GX₂₅KAALTX₂₆X₂₇GX₂₈QX₂₉EDEAX₃₀YX₃₁CX₃₂LWYSNX₃₃WVFGGGTKLTVL, where X₁ is T or A; X₂ is P or S; X₃ is S or A; X₄ is V or T; X₅ is G or E; X₆ is G, R, or A; X₇ is S or T; X₈ is G or S; X₉ is N or Y; X₁₀ is P or A; X₁₁ is Q or E; X₁₂ is G or D; X₁₃ is Q or H; X₁₄ is A or L; X₁₅ is P or F; X₁ is R or T; X₁₇ is G or A; X₁₈ is K or D; X₁₉ is F or M; X₂₀ is L or R; X₂₁ is A, P or V; X₂₂ is P or S; X₂₃ is Tor V; X₂₄ is L or 1; X₂₅ is G or D; X₂₆ is L or 1; X₂₇ is S or T; X₂₈ is V or A; X₂₉ is P or T; X₃₀ is E or I; X₃₁ is Y or F; X₃₂ is V, A, or T; X₃₃ is R or L; and
iii) wherein the VH and/or VL region sequence comprises one or more amino acid substitutions selected from
   a. N30 (preferably S), X₆34V, X₆34F, Q39 (E, K, R, D, preferably E), L45 (M or V, preferably M), D64 (preferably E), X₇65A, X₁₂81V, X₁₂81T, X₁₂811, V99 (A or L, preferably A), H101 (preferably A or N), X₁₄102E, F104 (preferably I), and N106 (preferably S or T) in the VH region sequence in i); and
   b. L20 (preferably I or M), V38 (preferably I), X₁₁40R, X₁₁40K, X₂₄69 (S or E, preferably S), X₃₂91L, X₃₃93 (preferably Y), and G102 (preferably S) in the VL region sequence in ii).

In this embodiment, preferred VH and VL region sequences comprised in the binding domain and mediating the binding to the human CD3ε chain are described. As evident from the above, the VH and VL region sequences of i) and ii) comprise various placeholders, wherein the same hierarchy and nomenclature applies for the placeholders as recited herein above in the context of the CDRs comprised in said VH and VL region of i) and ii). As is evident from the sequences for the VH and VL region, said CDRs described herein above are part of said VH and VL region sequence. Accordingly, the preferred embodiments of said CDRs of i) and ii) as described herein above also apply to this preferred embodiment.

The VH region of i) comprises or consists of the sequence of EVX₁LX₂ESGGGLX₃QPX₄GSLKLSCAASGFTFNX₅YAX₆NWVRQAPGKGLEWVARIRSKYNN YATYYADX₇VKX₈RFTISRDDSX₉X₁₀X₁₁X₁₂YLQMNNLKTEDTAX₁₃YYCVRHX₁₄NFGNSYX₁₅S X₁₆X₁₇AYWGQGTLVTVSX₁₈, where X₁ is Q or K; X₂ is V or L; X₃ is V or E; X₄ is G or K; X₅ is K, V, S, G, R, T, or I; X₆ is M or I; X₇ is S or Q; X₈ is D, G, K, S, or E; X₉ is K or Q; X₁₀ is N or S; X₁₁ is T or 1; X₁₂ is A or L; X₁₃ is V or M; X₁₄ is G, R or A; X₁₅ is 1, L, V, or T; X₁₆ is Y, W or F; X₁₇ is W, F or Y; and X₁₈ is S or A.

The CDR sequences within said VH region are as follows: a CDR-H1 sequence comprising or consisting of X₅YAX₆N; a CDR-H2 sequence comprising or consisting of RIRSKYNNYATYYADX₇VKX₈; and a CDR-H3 sequence comprising or consisting of HX₁₄NFGNSYX₁₅SX₁₆X₁₇AY. Preferred examples of said CDR-H1 sequence X₅YAX₆N are selected from KYAMN, VYAMN, SYAMN, GYAMN, RYAMN, TYAMN, IYAMN, and KYAIN, wherein KYAMN or KYAIN is most preferred. Preferred examples of said CDR-H2 sequence RIRSKYNNYATYYADX₇VKX₈ are selected from RIRSKYNNYATYYADSVKD, RIRSKYNNYATYYADSVKK, RIRSKYNNYATYYADSVKS, RIRSKYNNYATYYADSVKE, RIRSKYNNYATYYADSVKG, and RIRSKYNNYATYYADQVKD, wherein RIRSKYNNYATYYADSVKD is most preferred. Preferred examples of said CDR-H3 sequence HX₁₄NFGNSYX₁₅SX₁₆X₁₇AY are selected from HGNFGNSYISYWAY, HGNFGNSYLSWWAY, HGNFGNSYTSYYAY, HRNFGNSYLSWFAY, HGNFGNSYVSFFAY, HGNFGNSYISWWAY, HGNFGNSYVSWWAY, HGNFGNSYLSYFAY, HGNFGNSYLSFWAY, HANFGNSYISYWAY, and HGNFGNSYVSWFAY, wherein HGNFGNSYISYWAY is most preferred.

For X₁, the amino acid Q is preferred. For X₂, the amino acid V is preferred. For X₃, the amino acid V is preferred. For X₄, the amino acid G is preferred. For X₅, the amino acid K is most preferred, and for X₆, the amino acid M is most preferred. A preferred combination for X₅ and X₆ is M for X₆ in combination with any of the amino acid residues of X₅, such as the combinations for X₅ and X₆, respectively, selected from K and M, V and M, S and M, most preferred the combination K and M. Another preferred combination is K and I, for X₅ and X₆, respectively. Preferred examples of said CDR-H1 sequence X₅YAX₆N are selected from KYAMN, VYAMN, SYAMN, GYAMN, RYAMN, TYAMN, IYAMN, and KYAIN, wherein KYAMN or KYAIN is most preferred. For X₇, the amino acid S is most preferred, and for X₈, the amino acid D is most preferred. A preferred combination is S for X₇ in combination with any of the amino acid residues of X₈, such as the combinations for X₇ and X₈, respectively, selected from S and D, S and G, S and K, S and S, most preferred the combination S and D. Preferred examples of said CDR-H2 sequence RIRSKYNNYATYYADX₇VKX₈ are selected from RIRSKYNNYATYYADSVKD, RIRSKYNNYATYYADSVKK, RIRSKYNNYATYYADSVKS, RIRSKYNNYATYYADSVKE, RIRSKYNNYATYYADSVKG, and RIRSKYNNYATYYADQVKD, wherein RIRSKYNNYATYYADSVKD is most preferred. For X₉, the amino acid K is preferred. For X₁₀, the amino acid N is preferred. For X₁₁, the amino acid T is preferred. For X₁₂, the amino acid A is preferred. For X₁₃, the amino acid V is preferred. For X₁₄, the amino acid G is most preferred; for X₁₅, the amino acid I is most preferred; for X₁₆, the amino acid Y is most preferred; and for X₁₇, the amino acid W is most preferred. Preferred examples of said CDR-H3 sequence HX₁₄NFGNSYX₁₅SX₁₆X₁₇AY are selected from HGNFGNSYISYWAY, HGNFGNSYLSWWAY, HGNFGNSYTSYYAY, HRNFGNSYLSWFAY, HGNFGNSYVSFFAY, HGNFGNSYISWWAY, HGNFGNSYVSWWAY, HGNFGNSYLSYFAY, HGNFGNSYLSFWAY, HANFGNSYISYWAY, and HGNFGNSYVSWFAY, wherein HGNFGNSYISYWAY is most preferred. For X₁₈, the amino acid sequence S is preferred.

The VL region of ii) comprises or consists of the sequence of QX₁VVTQEX₂X₃LTX₄SPGX₅TVTLTCX₆SSTGAVTX₇X₈X₉YX₁₀NWVQX₁ KPX₁₂X₁₃X₁₄X₁₅X₁₆GL IGX₁₇TX₁₈X₁₉X₂₀X₂₁X₂₂GX₂₃PARFSGSLX₂₄GX₂₅KAALTX₂₆X₂₇GX₂₈QX₂₉EDEAX₃₀YX₃₁CX₃₂LW YSNX₃₃WVFGGGTKLTVL, where X₁ is T or A; X₂ is P or S; X₃ is S or A; X₄ is V or T; X₅ is G or E; X₆ is G, R, or A; X₇ is S or T; X₈ is G or S; X₉ is N or Y; X₁₀ is P or A; X₁₁ is Q or E; X₁₂ is G or D; X₁₃ is Q or H; X₁₄ is A or L; X₁₅ is P or F; X₁₆ is R or T; X₁₇ is G or A; X₁₈ is K or D; X₁₉ is F or M; X₂₀ is L or R; X₂₁ is A, P or V; X₂₂ is P or S; X₂₃ is T or V; X₂₄ is L or I; X₂₅ is G or D; X₂₆ is L or I; X₂₇ is S or T; X₂₈ is V or A; X₂₉ is P or T; X₃₀ is E or I; X₃₁ is Y or F; X₃₂ is V, A, or T; and X₃₃ is R or L.

The CDR sequences within said VL region are as follows: a CDR-L1 sequence comprising or consisting of X₆SSTGAVTX₇X₈X₉YX₁₀N; a CDR-L2 sequence comprising or consisting of X₁₇TX₁₈X₁₉X₂₀X₂₁X₂₂; and a CDR-L3 sequence comprising or consisting of X₃₂LWYSNX₃₃WV. Preferred examples of the CDR-L1 sequence are selected from GSSTGAVTSGNYPN, RSSTGAVTSGYYPN, GSSTGAVTSGYYPN, ASSTGAVTSGNYPN, and RSSTGAVTTSNYAN, wherein GSSTGAVTSGNYPN is most preferred. Preferred examples of the CDR-L2 sequence are selected from GTKFLAP, ATDMRPS, GTKFLVP, and GTNKRAP, wherein GTKFLAP is most preferred. Preferred examples of the CDR-L3 sequence are selected from VLWYSNRWV, ALWYSNRWV, TLWYSNRWV, and ALWYSNLWV, wherein VLWYSNRWV is most preferred.

For X₁, the amino acid T is preferred. For X₂, the amino acid P is preferred. For X₃, the amino acid S is preferred. For X₄, the amino acid V is preferred. For X₅, the amino acid G is preferred. For X₆, the amino acid G is most preferred. For X₇, the amino acid S is most preferred. For X₈, the amino acid G is most preferred. For X₉, the amino acid N is most preferred. For X₁₀, the amino acid P is most preferred. X₆ to X₁₀ are part of the CDR-L1 sequence within the VL region. Preferred examples of said CDR-L1 sequence are selected from GSSTGAVTSGNYPN, RSSTGAVTSGYYPN, GSSTGAVTSGYYPN, ASSTGAVTSGNYPN, and RSSTGAVTTSNYAN, wherein GSSTGAVTSGNYPN is most preferred. For X₁₁, the amino acid Q is preferred. For X₁₂, the amino acid G is preferred. For X₁₃, the amino acid Q is preferred. For X₁₄, the amino acid A is preferred. For X₁₅, the amino acid P is preferred. For X₁₆, the amino acid R is preferred. For X₁₇, the amino acid G is preferred. For X₁₈, the amino acid K is preferred. For X₁₉, the amino acid F is preferred. For X₂₀, the amino acid L is preferred. For X₂₁, the amino acid A is preferred. For X₂₂, the amino acid P is preferred. X₁₇ to X₂₂ are part of the CDR-L2 sequence of the VL region. Preferred examples of said CDR-L2 sequence are selected from GTKFLAP, ATDMRPS, GTKFLVP, and GTNKRAP, wherein GTKFLAP is most preferred. For X₂₃, the amino acid T is preferred. For X₂₄, the amino acid L is preferred. For X₂₅, the amino acid G is preferred. For X₂₆, the amino acid L is preferred. For X₂₇, the amino acid S is preferred. For X₂₈, the amino acid V is preferred. For X₂₉, the amino acid P is preferred. For X₃₀, the amino acid E is preferred. For X₃₁, the amino acid Y is preferred. For X₃₂, the amino acid V is preferred. For X₃₃, the amino acid R is preferred. X₃₂ and X₃₃ are part of the CDR-L3 sequence of the VL region. Preferred examples of said CDR-L3 sequence are selected from VLWYSNRWV, ALWYSNRWV, TLWYSNRWV, and ALWYSNLWV, wherein VLWYSNRWV is most preferred.

In a more preferred embodiment, the VH region of i) comprises or consists of the sequence of EVQLVESGGGLX₁QPGGSLKLSCAASGFTFNX₂YAX₃NWVRQAPGKGLEWVARIRSKYNNY ATYYADX₄VKX₅RFTISRDDSKNTAYLQMNNLKTEDTAVYYCVRHX₆NFGNSYX₇SX₈X₉AYW GQGTLVTVSS, where X₁ is V or E; X₂ is K, V, S, G, R, or I; X₃ is M or I; X₄ is S or Q; X₅ is D, G, K, S, or E; X₆ is G, R or A; X₇ is I, L, V, or T; X₈ is Y, W or F; and X₉ is W or F; and the VL region of ii) comprises or consists of the sequence of QTVVTQEPSLTVSPGGTVTLTCX₁SSTGAVTSGX₂YPNWVQQKPGQAPRGLIGX₃TX₄X₅X₆X₇ X₃GTPARFSGSLLGGKAALTLSGVQPEDEAEYYCX₉LWYSNRWVFGGGTKLTVL, where X, is G, R, or A; X₂ is N or Y; X₃ is G or A; X₄ is K or D; X₅ is F or M; X₆ is L or R; X₇ is A, P or V; X₈ is P or S; and X₉ is V, A, or T.

The VH region of i) comprises or consists of the sequence of EVQLVESGGGLX₁QPGGSLKLSCAASGFTFNX₂YAX₃NWVRQAPGKGLEWVARIRSKYNNY ATYYADX₄VKX₅RFTISRDDSKNTAYLQMNNLKTEDTAVYYCVRHX₆NFGNSYX₇SX₈X₉AYW GQGTLVTVSS, where X, is V or E; X₂ is K, V, S, G, R, or I; X₃ is M or I; X₄ is S or Q; X₅ is D, G, K, S, or E; X₆ is G, R or A; X₇ is I, L, V, or T; X₈ is Y, W or F; and X₉ is W or F.

The CDR sequences within said VH region are as follows: a CDR-H1 sequence comprising or consisting of X₂YAX₃N; a CDR-H2 sequence comprising or consisting of RIRSKYNNYATYYADX₄VKX₅; and a CDR-H3 sequence comprising or consisting of HX₆NFGNSYX₇SX₃X₉AY. Preferred examples of said CDR-H1 sequence X₂YAX₃N are selected from KYAMN, VYAMN, SYAMN, GYAMN, RYAMN, TYAMN, IYAMN, and KYAIN, wherein KYAMN or KYAIN is more preferred, wherein KYAMN is most preferred. Preferred examples of said CDR-H2 sequence RIRSKYNNYATYYADX₄VKX₅ are selected from RIRSKYNNYATYYADSVKD, RIRSKYNNYATYYADSVKK, RIRSKYNNYATYYADSVKS, RIRSKYNNYATYYADSVKE, RIRSKYNNYATYYADSVKG, and RIRSKYNNYATYYADQVKD, wherein RIRSKYNNYATYYADSVKD or RIRSKYNNYATYYADQVKD is more preferred, wherein RIRSKYNNYATYYADSVKD is most preferred. Preferred examples of said CDR-H3 sequence HX₆NFGNSYX₇SX₃X₉AY are selected from HGNFGNSYISYWAY, HGNFGNSYLSWWAY, HGNFGNSYTSYYAY, HRNFGNSYLSWFAY, HGNFGNSYVSFFAY, HGNFGNSYISWWAY, HGNFGNSYVSWWAY, HGNFGNSYLSYFAY, HGNFGNSYLSFWAY, HANFGNSYISYWAY, and HGNFGNSYVSWFAY, wherein HGNFGNSYISYWAY is most preferred.

For X₁, the amino acid V is preferred. For X₂, the amino acid K is most preferred, and for X₃, the amino acid M is most preferred. A preferred combination for X₂ and X₃ is M for X₃ in combination with any of the amino acid residues of X₂, such as the combinations for X₂ and X₃, respectively, selected from K and M, V and M, S and M, most preferred the combination K and M. Another preferred combination is K and I, for X₂ and X₃, respectively. Preferred examples of said CDR-H1 sequence X₂YAX₃N are selected from KYAMN, VYAMN, SYAMN, GYAMN, RYAMN, TYAMN, IYAMN, and KYAIN, wherein KYAMN or KYAIN is more preferred, wherein KYAMN is most preferred. For X₄, the amino acid S is preferred. For X₅, the amino acid D is most preferred. A preferred combination is S for X₄ in combination with any of the amino acid residues of X₅, such as the combinations for X₄ and X₅, respectively, selected from S and D, S and G, S and K, S and S, most preferred the combination S and D. Preferred examples of said CDR-H2 sequence RIRSKYNNYATYYADX₄VKX₅ are selected from RIRSKYNNYATYYADSVKD, RIRSKYNNYATYYADSVKK, RIRSKYNNYATYYADSVKS, RIRSKYNNYATYYADSVKE, RIRSKYNNYATYYADSVKG, and RIRSKYNNYATYYADQVKD, wherein RIRSKYNNYATYYADSVKD or RIRSKYNNYATYYADQVKD is more preferred, wherein RIRSKYNNYATYYADSVKD is most preferred. For X₆, the amino acid G is most preferred; for X₇, the amino acid I is most preferred; for X₈, the amino acid Y is most preferred; and for X₉, the amino acid W is most preferred. Preferred examples of said CDR-H3 sequence HX₆NFGNSYX₇SX₈X₉AY are selected from HGNFGNSYISYWAY, HGNFGNSYLSWWAY, HGNFGNSYTSYYAY, HRNFGNSYLSWFAY, HGNFGNSYVSFFAY, HGNFGNSYISWWAY, HGNFGNSYVSWWAY, HGNFGNSYLSYFAY, HGNFGNSYLSFWAY, HANFGNSYISYWAY, and HGNFGNSYVSWFAY, wherein HGNFGNSYISYWAY is most preferred.

The VL region of ii) comprises or consists of the sequence of QTVVTQEPSLTVSPGGTVTLTCX₁SSTGAVTSGX₂YPNWVQQKPGQAPRGLIGX₃TX₄X₅X₆X₇ X₈GTPARFSGSLLGGKAALTLSGVQPEDEAEYYCX₉LWYSNRWVFGGGTKLTVL, where X, is G, R, or A; X₂ is N or Y; X₃ is G or A; X₄ is K or D; X₅ is F or M; X₆ is L or R; X₇ is A, P or V; X₈ is P or S; and X₉ is V, A, or T.

The CDR sequences within said VL region are as follows: a CDR-L1 sequence comprising or consisting of X₁SSTGAVTSGX₂YPN; a CDR-L2 sequence comprising or consisting of X₃TX₄X₅X₆X₇X₈; and a CDR-L3 sequence comprising or consisting of X₉LWYSNRWV. Preferred examples of the CDR-L1 sequence are selected from GSSTGAVTSGNYPN, RSSTGAVTSGYYPN, GSSTGAVTSGYYPN, ASSTGAVTSGNYPN, and RSSTGAVTTSNYAN, wherein GSSTGAVTSGNYPN is most preferred. Preferred examples of the CDR-L2 sequence are selected from GTKFLAP, ATDMRPS, GTKFLVP, and GTNKRAP, wherein GTKFLAP is most preferred. Preferred examples of the CDR-L3 sequence are selected from VLWYSNRWV, ALWYSNRWV, TLWYSNRWV, and ALWYSNLWV, wherein VLWYSNRWV is most preferred.

For X₁, the amino acid G is most preferred. For X₂, the amino acid N is preferred. X₁ and X₂ are part of the CDR-L1 sequence X₁SSTGAVTSGX₂YPN within the VL region. Preferred examples of said CDR-L1 sequence are selected from GSSTGAVTSGNYPN, RSSTGAVTSGYYPN, GSSTGAVTSGYYPN, ASSTGAVTSGNYPN, and RSSTGAVTTSNYAN, wherein GSSTGAVTSGNYPN is most preferred. For X₃, the amino acid G is preferred. For X₄, the amino acid K is preferred. For X₅, the amino acid F is preferred. For X₆, the amino acid L is preferred. For X₇, the amino acid A is most preferred. For X₈, the amino acid P is preferred. X₃ to X₈ are part of the CDR-L2 sequence X₃TX₄X₅X₆X₇X₈ of the VL region. Preferred examples of said CDR-L2 sequence are selected from GTKFLAP, ATDMRPS, GTKFLVP, and GTNKRAP, wherein GTKFLAP is most preferred. For X₉, the amino acid V is most preferred. X₉ is part of the CDR-L3 sequence X_{L}LWYSNRWV of the VL region. Preferred examples of said CDR-L3 sequence are selected from VLWYSNRWV, ALWYSNRWV, TLWYSNRWV, and ALWYSNLWV, wherein VLWYSNRWV is most preferred.

In another preferred embodiment, the VH region of ii) comprises or consists of the sequence of EVQLVESGGGLX₁QPGGSLKLSCAASGFTFNX₂YAX₃NWVRQAPGKGLEWVARIRSKYNNY ATYYADX₄VKX₅RFTISRDDSKNTAYLQMNNLKTEDTAVYYCVRHX₆NFGNSYX₇SX₃X₉AYW GQGTLVTVSS, where X, is V or E; X₂ is K, V, S, R, or I; X₃ is M or I; X₄ is S or Q; X₅ is D, G, K or S; X₆ is G or A; X₇ is I, L, V, or T; X₈ is Y, W or F; and X₉ is W or F; and the VL region of ii) comprises or consists of the sequence of QTVVTQEPSLTVSPGGTVTLTCX₁SSTGAVTSGX₂YPNWVQQKPGQAPRGLIGGTKFLX₃PG TPARFSGSLLGGKAALTLSGVQPEDEAEYYCX₄LWYSNRWVFGGGTKLTVL, where X, is G or A; X₂ is N or Y; X₃ is A or V; and X₄ is V, A, or T.

The VH region of ii) comprises or consists of the sequence of EVQLVESGGGLX₁QPGGSLKLSCAASGFTFNX₂YAX₃NWVRQAPGKGLEWVARIRSKYNNY ATYYADX₄VKX₅RFTISRDDSKNTAYLQMNNLKTEDTAVYYCVRHX₆NFGNSYX₇SX₃X₉AYW GQGTLVTVSS, where X, is V or E; X₂ is K, V, S, R, or I; X₃ is M or I; X₄ is S or Q; X₅ is D, G, K or S; X₆ is G or A; X₇ is I, L, V, or T; X₈ is Y, W or F; and X₉ is W or F.

The CDR sequences within said VH region are as follows: a CDR-H1 sequence comprising or consisting of X₂YAX₃N; a CDR-H2 sequence comprising or consisting of RIRSKYNNYATYYADX₄VKX₅; and a CDR-H3 sequence comprising or consisting of HX₆NFGNSYX₇SX₃X₉AY. Preferred examples of said CDR-H1 sequence X₂YAX₃N are selected from KYAMN, VYAMN, SYAMN, RYAMN, IYAMN, and KYAIN, wherein KYAMN or KYAIN are more preferred, wherein KYAMN is most preferred. Preferred examples of said CDR-H2 sequence RIRSKYNNYATYYADX₄VKX₅ are selected from RIRSKYNNYATYYADSVKD, RIRSKYNNYATYYADSVKK, RIRSKYNNYATYYADSVKS, RIRSKYNNYATYYADSVKG, and RIRSKYNNYATYYADQVKD, wherein RIRSKYNNYATYYADSVKD or RIRSKYNNYATYYADQVKD are more preferred, wherein RIRSKYNNYATYYADSVKD is most preferred. Preferred examples of said CDR-H3 sequence HX₆NFGNSYX₇SX₃X₉AY are selected from HGNFGNSYISYWAY, HGNFGNSYLSWWAY, HGNFGNSYTSYYAY, HRNFGNSYLSWFAY, HGNFGNSYVSFFAY, HGNFGNSYISWWAY, HGNFGNSYVSWWAY, HGNFGNSYLSYFAY, HGNFGNSYLSFWAY, HANFGNSYISYWAY, and HGNFGNSYVSWFAY, wherein HGNFGNSYISYWAY or HANFGNSYISYWAY are more preferred, wherein HGNFGNSYISYWAY is most preferred.

For X₁, the amino acid V is preferred. For X₂, the amino acid K is most preferred, and for X₃, the amino acid M is most preferred. A preferred combination for X₂ and X₃ is M for X₃ in combination with any of the amino acid residues of X₂, such as the combinations for X₂ and X₃, respectively, selected from K and M, V and M, S and M, most preferred the combination K and M. Another preferred combination is K and I, for X₂ and X₃, respectively. Preferred examples of said CDR-H1 sequence X₂YAX₃N are selected from KYAMN, VYAMN, SYAMN, RYAMN, IYAMN, and KYAIN, wherein KYAMN or KYAIN are more preferred, wherein KYAMN is most preferred. For X₄, the amino acid S is preferred. For X₅, the amino acid D is most preferred. A preferred combination is S for X₄ in combination with any of the amino acid residues of X₅, such as the combinations for X₄ and X₅, respectively, selected from S and D, S and G, S and K, S and S, most preferred the combination S and D. Preferred examples of said CDR-H2 sequence RIRSKYNNYATYYADX₄VKX₅ are selected from RIRSKYNNYATYYADSVKD, RIRSKYNNYATYYADSVKK, RIRSKYNNYATYYADSVKS, RIRSKYNNYATYYADSVKG, and RIRSKYNNYATYYADQVKD, wherein RIRSKYNNYATYYADSVKD or RIRSKYNNYATYYADQVKD are more preferred, wherein RIRSKYNNYATYYADSVKD is most preferred are selected from RIRSKYNNYATYYADSVKD, RIRSKYNNYATYYADSVKK, RIRSKYNNYATYYADSVKS, RIRSKYNNYATYYADSVKE, RIRSKYNNYATYYADSVKG, and RIRSKYNNYATYYADQVKD, wherein RIRSKYNNYATYYADSVKD is most preferred. For X₈, the amino acid G is most preferred; for X₇, the amino acid I is most preferred; for X₈, the amino acid Y is most preferred; and for X₉, the amino acid W is most preferred. Preferred examples of said CDR-H3 sequence HX₆NFGNSYX₇SX₈X₉AY are selected from HGNFGNSYISYWAY, HGNFGNSYLSWWAY, HGNFGNSYTSYYAY, HRNFGNSYLSWFAY, HGNFGNSYVSFFAY, HGNFGNSYISWWAY, HGNFGNSYVSWWAY, HGNFGNSYLSYFAY, HGNFGNSYLSFWAY, HANFGNSYISYWAY, and HGNFGNSYVSWFAY, wherein HGNFGNSYISYWAY is most preferred.

The VL region of ii) comprises or consists of the sequence of QTVVTQEPSLTVSPGGTVTLTCX₁SSTGAVTSGX₂YPNWVQQKPGQAPRGLIGGTKFLX₃PG TPARFSGSLLGGKAALTLSGVQPEDEAEYYCX₄LWYSNRWVFGGGTKLTVL, where X, is G or A; X₂ is N or Y; X₃ is A or V; and X₄ is V, A, or T.

The CDR sequences within said VL region are as follows: a CDR-L1 sequence comprising or consisting of X₁SSTGAVTSGX₂YPN; a CDR-L2 sequence comprising or consisting of GTKFLX₃P; and a CDR-L3 sequence comprising or consisting of X₄LWYSNRWV. Preferred examples of the CDR-L1 sequence are selected from GSSTGAVTSGNYPN, RSSTGAVTSGYYPN, GSSTGAVTSGYYPN, ASSTGAVTSGNYPN, and RSSTGAVTTSNYAN, wherein GSSTGAVTSGNYPN is most preferred. Preferred examples of the CDR-L2 sequence are selected from GTKFLAP, ATDMRPS, GTKFLVP, and GTNKRAP, wherein GTKFLAP is most preferred. Preferred examples of the CDR-L3 sequence are selected from VLWYSNRWV, ALWYSNRWV, TLWYSNRWV, and ALWYSNLWV, wherein VLWYSNRWV is most preferred.

For X₁, the amino acid G is preferred. For X₂, the amino acid N is preferred. X₁ and X₂ are part of the CDR-L1 sequence X₁SSTGAVTSGX₂YPN within the VL region. Preferred examples of said CDR-L1 sequence are selected from GSSTGAVTSGNYPN, RSSTGAVTSGYYPN, GSSTGAVTSGYYPN, ASSTGAVTSGNYPN, and RSSTGAVTTSNYAN, wherein GSSTGAVTSGNYPN is most preferred. X₃ is part of the CDR-L2 sequence GTKFLX₃P of the VL region. Said CDR-L2 sequence can either be GTKFLAP or GTKFLVP, wherein GTKFLAP is preferred. For X₃, the amino acid A is preferred. X₄ is part of the CDR-L3 sequence X₄LWYSNRWV of the VL region. Preferred examples of said CDR-L3 sequence are selected from VLWYSNRWV, ALWYSNRWV, and TLWYSNRWV, wherein VLWYSNRWV is most preferred. For X₄, the amino acid V is most preferred.

In a further preferred embodiment, the VH region of i) comprises or consists of the sequence of EVQLVESGGGLVQPGGSLKLSCAASGFTFNX₁YAMNWVRQAPGKGLEWVARIRSKYNNYA TYYADSVKX₂RFTISRDDSKNTAYLQMNNLKTEDTAVYYCVRHGNFGNSYX₃SX₄WAYWGQ GTLVTVSS, where X, is K or S; X₂ is D or G; X₃ is I or V; and X₄ is Y or W; and the VL region of ii) comprises or consists of the sequence of QTVVTQEPSLTVSPGGTVTLTCGSSTGAVTSGX₁YPNWVQQKPGQAPRGLIGGTKFLAPGT PARFSGSLLGGKAALTLSGVQPEDEAEYYCX₂LWYSNRWVFGGGTKLTVL, where X, is N or Y; and X₂ is V or A.

The VH region of i) comprises or consists of the sequence of EVQLVESGGGLVQPGGSLKLSCAASGFTFNX₁YAMNWVRQAPGKGLEWVARIRSKYNNYA TYYADSVKX₂RFTISRDDSKNTAYLQMNNLKTEDTAVYYCVRHGNFGNSYX₃SX₄WAYWGQ GTLVTVSS, where X, is K or S, wherein K is preferred; X₂ is D or G, wherein D is preferred; X₃ is I or V, wherein I is preferred; and X₄ is Y or W, wherein Y is preferred.

The CDR sequences within said VH region are as follows: a CDR-H1 sequence comprising or consisting of X₁YAMN; a CDR-H2 sequence comprising or consisting of RIRSKYNNYATYYADSVKX₂; and a CDR-H3 sequence comprising or consisting of HGNFGNSYX₃SX₄WAY. Said CDR-H1 sequence X₁YAMN is selected from KYAMN or SYAMN, wherein KYAMN is preferred. Preferred examples of said CDR-H2 sequence RIRSKYNNYATYYADSVKX₂ are selected from RIRSKYNNYATYYADSVKD and RIRSKYNNYATYYADSVKG, wherein RIRSKYNNYATYYADSVKD is preferred. Said CDR-H3 sequence HGNFGNSYX₃SX₄WAY is HGNFGNSYISYWAY or HGNFGNSYVSWWAY, wherein HGNFGNSYISYWAY is preferred.

The VL region of ii) comprises or consists of the sequence of QTVVTQEPSLTVSPGGTVTLTCGSSTGAVTSGX₁YPNWVQQKPGQAPRGLIGGTKFLAPGT PARFSGSLLGGKAALTLSGVQPEDEAEYYCX₂LWYSNRWVFGGGTKLTVL, where X, is N or Y, wherein N preferred; and X₂ is V or A, wherein V preferred.

The CDR sequences within said VL region are as follows: a CDR-L1 sequence comprising or consisting of GSSTGAVTSGX₁YPN; a CDR-L2 sequence comprising or consisting of GTKFLAP; and a CDR-L3 sequence comprising or consisting of X₂LWYSNRWV. The CDR-L1 sequence is GSSTGAVTSGNYPN or GSSTGAVTSGYYPN, wherein GSSTGAVTSGNYPN is preferred. The CDR-L3 sequence is VLWYSNRWV or ALWYSNRWV, wherein VLWYSNRWV is preferred.

In a preferred embodiment, the VH region of i) comprises or consists of the sequence as defined in SEQ ID NOs: 124, 172, 362, 552, 730, 928, 1106, 1296, 1474, 1664 or 1854; and the VL region of ii) comprises or consists of the sequence of as defined in SEQ ID NOs: 125, 173, 363, 553, 731, 929, 1107, 1297, 1475, 1665, or 1855. Preferred combinations of VH and VL regions of i) and ii) are defined in SEQ ID NOs: 124 and 125 (combination of binder termed “2B2”), 172 and 173 (“A2J”), 362 and 363 (“E1L”), 552 and 553 (“E2M”), 730 and 731 (“F12Q”), 928 and 929 (“F6A”), 1106 and 1107 (“F70”), 1296 and 1297 (“G4H”), 1474 and 1475 (“H1E”), 1664 and 1665 (“H2C”), or 1854 and 1855 (“I2C”). Preferred is that the CD3epsilon binding domain comprises or consists of a combination of VH and VL region of i) and ii) selected from the group consisting of SEQ ID NOs: 124 and 125, 172 and 173, 362 and 363, 552 and 553, 730 and 731, 928 and 929, 1106 and 1107, 1296 and 1297, 1474 and 1475, 1664 and 1665, and 1854 and 1855. More preferred is that the CD3epsilon binding domain comprises or consists of a combination of VH and VL region of i) and ii) selected from the group consisting of SEQ ID NOs: 124 and 125, 362 and 363, 730 and 731, 928 and 929, 1106 and 1107, 1296 and 1297, 1474 and 1475, 1664 and 1665, and 1854 and 1855. Even more preferred is that the CD3epsilon binding domain comprises or consists of a combination of VH and VL region of i) and ii) selected from the group consisting of SEQ ID NOs: 124 and 125, 730 and 731, 1664 and 1665, and 1854 and 1855. In a yet even more preferred embodiment, the CD3epsilon binding domain comprises or consists of a combination of VH and VL region of i) and ii) selected from the group consisting of SEQ ID NOs: 730 and 731, 1664 and 1665, and 1854 and 1855.

In the most preferred embodiment, the VH region of i) comprises or consists of the sequence of EVQLVESGGGLVQPGGSLKLSCAASGFTFNKYAMNWVRQAPGKGLEWVARIRSKYNNYA TYYADSVKDRFTISRDDSKNTAYLQMNNLKTEDTAVYYCVRHGNFGNSYISYWAYWGQGT LVTVSS (SEQ ID NO: 1854); and the VL region of ii) comprises or consists of the sequence of QTVVTQEPSLTVSPGGTVTLTCGSSTGAVTSGNYPNWVQQKPGQAPRGLIGGTKFLAPGT PARFSGSLLGGKAALTLSGVQPEDEAEYYCVLWYSNRWVFGGGTKLTVL (SEQ ID NO: 1855).

As outlined herein above in relation to amino acid substitutions in the CDR sequences and also applying to this embodiment, the VH region of i) and/or VL region of ii) comprise one or more amino acid substitutions as specified herein above. Accordingly, at least one of the recited amino acid substitutions of the variable regions is comprised in said binding domain binding to an extracellular epitope of the human CD3ε chain. Said at least one or one or more amino acid substitution, in the case that only one amino acid substation is present, can be present in a CDR sequence in said VH or said VL region or, in case more than one amino acid substitution is present, in CDRs in both the VH and the VL region or restricted to CDR sequences in only the VH or the VL region. In certain embodiments where there are more than one amino acid substitutions, the substitutions are in the VH and the VL region or, alternatively, only in one variable region. Preferably, the substitutions are in the VH and the VL region.

Specifically, the VH of i) and/or VL region sequence of ii) comprises one or more amino acid substitutions selected from a. N30 (preferably S), X₆34V, X₆34F, Q39 (E, K, R, D, preferably E), L45 (M or V, preferably M), D64 (preferably E), X₇65A, X₁₂81V, X₁₂81T, X₁₂811, V99 (A or L, preferably A), H101 (preferably A or N), X₁₄102E, F104 (preferably I), N106 (preferably S or T) in the VH region sequence in i); and b. L20 (preferably I or M), V38 (1, L, M, F or Y, preferably I), X₁₁40R, X₁₁40K, X₂₄69 (S or E, preferably S), X₃₂91L, X₃₃93 (preferably Y), G102 (preferably S) in the VL region sequence in ii).

In the VH region sequence, the amino acid substitution at position 30 is, preferably, S (hence, can be written as N30S; same is true for the subsequent substitutions given according to their position number). The amino acid substitution at position 34 is V or F. Preferably, position 34 of the VH region sequence comprises an I or M, most preferred an 1. The amino acid substitution at position 39 is E, K, R, D, preferably E. The amino acid substitution at position 45 is preferably M or V, more preferred M. The amino acid substitution at position 64 is preferably E. The amino acid substitution at position 65 is A. The amino acid substitution at position 81 is V, T or I, preferably V. The amino acid substitution at position 99 is preferably A or L, more preferred A. The amino acid substitution at position 101 is preferably A or N, more preferred A (H1A). The amino acid substitution at position 102 is E. The amino acid substitution at position 104 is preferably 1. The amino acid substitution at position 106 is preferably S or T, more preferred S.

Notwithstanding the above, in the CDR-H2 sequence within the VH region sequence, the amino acid substitution at position 64 is, preferably, D15E, or the amino acid substitution at position 65 with amino acid A. Preferably, said CDR-H2 comprises the amino acid residue substitution at position 65 with amino acid A; in this case, a preferred amino acid for the last amino acid of CDR-H2 is D or G, preferred D. Hence, a preferred CDR-H2 sequence is RIRSKYNNYATYYADAVKD or RIRSKYNNYATYYADAVKG, with RIRSKYNNYATYYADAVKD more preferred. In case CDR-H2 contains two amino acid substitutions, the combination of the amino acid E at position 64 and amino acid A at position 64 are preferred; in this case, a preferred amino acid for the last amino acid of CDR-H2 is G. Hence, a preferred CDR-H2 sequence for the combination of the amino acid E at position 64 and amino acid A at position 65 is RIRSKYNNYATYYAEAVKG.

In the CDR-H3 sequence of the VH sequence, the amino acid substitutions are at position 101, preferably a substitution with amino acid A or N, more preferably A, at position 102 with amino acid E, at position 104, preferably with amino acid I, and at position 106, preferably with amino acid S or T. Preferably, CDR-H3 comprises the amino acid A or N at position 101, the amino acid S or T at position 106; in this case and if placeholders are present in the CDR-H3 sequence at the indicated positions, preferred amino acids for X₁₄ are G or A, most preferred G; for X₁₅ is I; for X₁₆ is Y; and for X₁₇ are W or Y. In case CDR-H3 comprises two amino acid substitutions, preferred combinations are the amino acid A at position 101 and the amino acid S at position 106; and the amino acid N at position 101 and the amino acid T at position 106; in this case and if placeholders are present in the CDR-H3 sequence at the indicated positions, a preferred amino acid for X₁₄ is G or A, most preferred G; for X₁₅ is I, for X₁₆ is Y, and for X₁₇ are W and F. In case CDR-H3 comprises three amino acid substitutions, preferred combinations are the amino acid A at position 101, the amino acid I at position 104, and the amino acid S at position 106; and the amino acid N at position 101, the amino acid I at position 104 and the amino acid T at position 106, in this case and if placeholders are present in the CDR-H3 sequence at the indicated positions, a preferred amino acid for X₁₅ is I, for X₁₆ is Y, and for X₁₇ are Wand F. In case CDR-H3 comprises four amino acid substitutions, a preferred combination is the amino acid N at position 101, the amino acid E at position 102, the amino acid I at position 104, and the amino acid T at position 106; in this case and if placeholders are present in the CDR-H3 sequence at the indicated positions, a preferred amino acid for X₁₅ is I, for X₁₆ is Y, and for X₁₇ is W. Hence, preferred CDR-H3 sequences are selected from AGNFGSSYISYWAY, NENIGTSYISYWAY, AGNFGTSYISYWAY, NANFGTSYISYFAY, and AGNFGSSYISYFAY, with AGNFGSSYISYWAY and AGNFGSSYISYFAY most preferred.

In the VL region sequence, the amino acid substitution at position 20 is preferably I or M, more preferred I. The amino acid substitution at position 38 is preferably I, L, M, F or Y, more preferred with I. The amino acid substitution at position 40 is preferably K or R, more preferred K. The amino acid substitution at position 69 is preferably S or E, more preferred S. The amino acid substitution at position 91 is L. The amino acid substitution at position 93 is preferably Y. The amino acid substitution at position 102 is preferably S.

Notwithstanding the above, in the CDR-L3 sequence within the VL region sequence, the amino acid substitution at position 1 is with amino acid L or the amino acid substitution is W93, preferably W93Y. Preferably, the CDR-L3 sequence is VLYYSNRWV when CDR-L3 comprises an amino acid substitution. It is preferred, however, that CDR-L3 does not comprise said X₁1L and W93 amino acid substitution, in which case a preferred CDR-L3 sequence is VLYYSNRWV.

In accordance with the foregoing, the amino acid substitution H101A in the VH region (part of CDR-H3) is preferably the amino acid substitution, in case there is only one amino acid substitution present in the CD3epsilon binding domain, within the VH and/or VL region sequences as defined herein. Furthermore, in case of combinations of amino acid substitutions as defined in iii) preferably H101A in the VH region sequence is one of the amino acid substitutions of said combination.

In a preferred embodiment of the polypeptide or polypeptide construct of the invention, wherein in addition to said one amino acid substitution or a combination thereof as defined in iii) X₆ is I; X₃ is G; X₁₂ is L, X₁₄ is A, X₁₇ is F in said VH region sequence; and/or X₃₂ is A in said VL region sequence. In a preferred embodiment of the polypeptide or polypeptide construct of claim 1 of the invention, in addition to said one amino acid substitution or a combination thereof as defined in iii) X₆ is I; X₃ is G; X₁₄ is A, X₁₇ is F in said VH region sequence; and/or X₃₂ is A in said VL region sequence. The one amino acid substitution or combination thereof as defined herein is/are in addition combined with specific amino acids at specific positions in the CDRs. Hence, at least one of the recited amino acid substitutions in accordance with iii) is present and at least one of X₃ is G; X₁₄ is A, X₁₇ is F in said VH region sequence; and/or X₃₂ is A in said VL region sequence is additionally present in the CDRs. As previously stated herein above, a preferred amino acid for X₆ is I or X₃ is G in said VH region sequence, if only one additional amino acid is present in the CDRs, wherein it is most preferred that X₅ is I in said VH region sequence. A preferred combination of two of said additional amino acids is X₆ is I and X₁₇ is F in said VH region sequence; or X₃ is G and X₁₇ is F in said VH region sequence. A preferred combination of three of said additional amino acids is X₆ is I, X₁₄ is A, and X₁₇ is F in said VH region sequence; or X₆ is I, X₁₇ is F in said VH region sequence, and X₃₂ is A in said VL region sequence. Since the latter amino acids are to be present additionally, it is understood that those recited additional amino acids that overlap in position with the amino acid substitution of iii) cannot replace the overlapping amino acid substitution. For example, if the only amino acid substitution in accordance with iii) is either X₆34V or X₆34F in CDR-H1, said amino acid cannot be replaced by amino acid I in said CDR-H1 sequence, since there would be no amino acid substitution as defined in iii) remaining in the binding domain. In other terms, there must always be at least one of the amino acid substitutions as defined in iii) comprised in the polypeptide or polypeptide construct of the invention.

It is also understood that depending on the actual sequence of the VH and/or VL region sequences, one or more of said additional amino acids may already be part of the given variable sequence as defined herein above or, if this is not the case, said given variable region will be changed to exhibit said additional amino acid(s) in combination with one amino acid substitution or a combination thereof as recited in iii).

In a preferred embodiment of the polypeptide or polypeptide construct of the invention, it comprises combinations of said amino acid substitutions as defined in iii) of two, three, four, five, six, seven, eight, nine, ten, eleven, twelve or more amino acid substitutions.

In a preferred embodiment of the polypeptide or polypeptide construct of the invention, i) said one acid substitution is selected from a. X₆34V, Q39E, Q39K, Q39R, Q39D, L45M, L45V, X₁₂81V, X₁₂81T, X₁₂811, V99A, V99L, H101A, H101N in the VH region; and b. V381, V38L, V38M, V38F, V38Y, X₁₁40R, X₁₁40K, X₂₄69S, X₂₄69E, X₃₂91L, X₃₃93Y; ii) said combination of two or more amino acid substitutions are selected from X₆34F and A81V (which may also be referred to as X₁₂81V) in the VH region; Q39E in the VH region and X₁₁40K in the VL region; Q39E in the VH region and X₁₁40R in the VL region; Q39D in the VH region and X₁₁40K in the VL region; Q39D in the VH region and X₁₁40R in the VL region; L45V in the VH region and V38F in the VL region; L45V in the VH region and V38Y in the VL region; L45M in the VH region and X₁₁40K in the VL region; L20I and G102S in the VL region; A81V in the VH region and X₁₁40K in the VL region; iii) said combination of three or more amino acid substitutions are selected from Q39E in the VH region, X₁₁40K and G102S in the VL region; Q39E in the VH region, X₁₁40R and G102S in the VL region; Q39D in the VH region, X₁₁40K and G102S in the VL region; Q39D in the VH region, X₁₁40R and G102S in the VL region; L45M in the VH region, X₁₁40K and G102S in the VL region; X₇65A, H101A, and N106S in the VH region; L20I, X₁₁40K, and G102S in the VL region; iv) said combination of four or more amino acid substitutions are selected from Q39E in the VH region, L20I, X₁₁40K and G102S in the VL region; Q39E in the VH region, L20I, X₁₁40R and G102S in the VL region; Q39D in the VH region, L20I, X₁₁40K and G102S in the VL region; Q39D in the VH region, L20I, X₁₁40R and G102S in the VL region; L45M in the VH region, L20I, X₁₁40K and G102S in the VL region; L20I, X₁₁40K, X₂₄69S, and G102S in the VL region; A81V in the VH region, L20I, X₁₁40K, and G102S in the VL region; and/or v) said combination of five, six, seven, eight, nine, ten, or more amino acid substitutions are selected from X₇65A, A81V, V99A, N106S in the VH region, L20I, X₁₁40K, X₂₄69S, and G102S in the VL region; D64E, X₇65A, A81V, X₁₄102E, F104I, N106T in the VH region, L20I, X₂₄69S, and G102S in the VL region; L45M, X₇65A, N106T in the VH region, L20I, X₁₁40K, X₂₄69S, and G102S in the VL region; X₇65A, A81V, V99A, H101A, F104I, N106S in the VH region, L20I, X₁₁40K, X₂₄69S, and G102S in the VL region; X₇65A, A81V, V99A, H101A, N106S in the VH region; A81V, V99A in the VH region, L20I, X₁₁40K, X₂₄69S, and G102S in the VL region; Q39E, A81V in the VH region, L20I, X₁₁40K, and G102S in the VL region; L45V in the VH region, L20I, V38F, X₁₁40K and G102S in the VL region.

In a more preferred embodiment of the polypeptide or polypeptide construct of the invention, said combination of amino acid substitutions is selected from:
i) in the VH region
   a. X₇65A, A81V, V99A, H101A and N106S;
   b. D64E, X₇65A, A81V, H101N, X₁₄102E, F104I, and N106S;
   c. L45M, X₇65A, H101A, and N106T;
   d. L45M, X₇65A, H101A, and N106S;
   e. Q39E, X₇65A, H101N, and N106T;
   f. D64E, X₇65A, V99A, H101A, and N106T;
   g. X₇65A, A81V, V99A, H101A, X₁₄102E, F104I, and N106T;
   h. X₇65A, A81V, H101N, X₁₄102E, F104I, and N106S;
   i. D64E, X₇65A, A81V, H101A, and N106S;
   j. D64E, X₇65A, H101A, and N106T;
   k. X₇65A, V99A, H101A, and N106T;
   l. D64E, X₇65A, H101A, and N106S;
   m. D64E, X₇65A, A81V, V99A, H101A, and N106S;
   n. X₇65A, H101A and N106S;
   o. N30S, Q39E, D64E, X₇65A, A81V, H101A, X₁₄102E, F104I, and N106T;
   p. L45M, D64E, X₇65A, H101A, and N106T;
   q. N30S, L45M, X₇65A, A81V, H101A, and N106T;
   r. N30S, L45M, D64E, X₇65A, A81V, H101A, and N106S;
ii) in the VL region
   a. L20I, X₁₁40K, X₂₄69S, and G102S;
   b. L20I, X₂₄69S, and G102S;
   c. L20I, V381, X₁₁40K, X₂₄69E, G102S and W93Y;
   d. X₁₁40K and G102S;
   e. L20I, X₁₁40K, X₂₄69S, G102S and W93Y;
   f. L20M, X₁₁40K and X₂₄69E;
   g. L20I, V381, X₁₁40K, X₂₄69E and G102S;
   h. X₁₁40K, X₂₄69S and W93Y;
   i. X₁₁40K and X₂₄69S; and
iii) a combination of one amino acid substitution combination of i) and ii).

In yet another preferred embodiment of the polypeptide or polypeptide construct of the invention, said combination of amino acid substitution combinations of iii) is selected from:
a. X₇65A, A81V, V99A, H101A, N106S in the VH region, L20I, X₁₁40K, X₂₄69S, and G102S in the VL region;
b. D64E, X₇65A, A81V, H101N, X₁₄102E, F104I, N106S in the VH region, L20I, X₂₄69S, and G102S in the VL region:
c. L45M, X₇65A, H101A, N106T in the VH region, L20I, X₁₁40K, X₂₄69S, and G102S in the VL region;
d. L45M, X₇65A, H101A, N106S in the VH region, L20I, V381, X₁₁40K, X₂₄69E, G102S and W93Y in the VL region;
e. Q39E, X₇65A, H101N, N106T in the VH region, X₁₁40K and G102S in the VL region;
f. D64E, X₇65A, V99A, H101A, N106T in the VH region, X₁₁40K and G102S in the VL region;
g. X₇65A, A81V, V99A, H101A, X₁₄102E, F104I, N106T in the VH region, L20I, X₁₁40K, X₂₄69S, and G102S in the VL region;
h. X₇65A, A81V, H101N, X₁₄102E, F104I, N106S in the VH region, L20I, X₂₄69S, and G102S in the VL region;
i. D64E, X₇65A, A81V, H101A, N106S in the VH region, L20I, X₂₄69S, and G102S in the VL region;
j. D64E, X₇65A, H101A, N106T in the VH region, X₁₁40K and G102S in the VL region;
k. X₇65A, V99A, H101A, N106T in the VH region, X₁₁40K and G102S in the VL region;
l. D64E, X₇65A, H101A, N106S in the VH region, X₁₁40K and G102S in the VL region;
m. D64E, X₇65A, A81V, V99A, H101A, N106S in the VH region, L20I, X₁₁40K, X₂₄69S, G102S and W93Y in the VL region;
n. X₇65A, H101A N106S in the VH region, X₁₁40K and G102S in the VL region;
o. N30S, Q39E, D64E, X₇65A, A81V, H101A, X₁₄102E, F104I, N106T in the VH region, L20M, X₁₁40K and X₂₄69E in the VL region;
p. L45M, D64E, X₇65A, H101A, N106T in the VH region, L20I, V381, X₁₁40K, X₂₄69E and G102S in the VL region;
q. N30S, L45M, X₇65A, A81V, H101A, N106T in the VH region, X₁₁40K and X₂₄69S in the VL region; and
r. N30S, L45M, D64E, X₇65A, A81V, H101A, N106S in the VH region, X₁₁40K and X₂₄69S in the VL region.

In another preferred embodiment of the polypeptide or polypeptide construct of the invention, in said combination of amino acid substitution combinations defined in:
a. and m. X₆ is I in said VH region sequence;
b. and r. X₃ is G in said VH region sequence;
c. X₁₂ is L in said VH region;
d. X₁₂ is L and X₁₇ is F in said VH region;
e. X₆ is I, X₁₄ is A and X₁₇ is F in said VH region sequence;
f. X₆ is I in said VH region sequence; and X₃₂ is A in said VL region sequence;
g. X₆ is I in said VH region sequence;
h. and i. X₃ is G in said VH region sequence;
j., l. and n. X₆ is I and X₁₇ is F in said VH region sequence; and X₃₂ is A in said VL region sequence;
k. X₆ is I said VH region sequence; and X₃₂ is A in said VL region sequence;
p. X₁₂ is L in said VH region; or
q. X₃ is G and X₁₇ is F in said VH region sequence.

Examples of VH and VL region sequences as defined in this embodiment are defined by SEQ ID NOs: 2012 and 2013; 2020 and 2021; 2028 and 2029; 2036 and 2037; 2044 and 2045; 26 and 27; 34 and 35; 42 and 43; 50 and 51; 58 and 59; 66 and 67; 74 and 75; 82 and 83; 90 and 91; 98 and 99; 100 and 101; 108 and 109; 116 and 117. Table 2 evidences the increase of the aforementioned VH and VL region sequence combinations of the CD3epsilon binding domain in temperature stability over the CD3epsilon binding domain comprising a VH and VL combination defined as “I2C” (SEQ ID NOs: 1854 and 1855, respectively).

In accordance with the invention, the binding domain of the polypeptide or polypeptide construct of the invention that binds to an extracellular epitope of the human CD3ε chain and comprising or consisting of a VH region and a VL region as defined herein comprises at least one of the following CDR sequences or a combination thereof as defined in SEQ ID NOs: 1878, 1908, 1989, and 2003. Said sequences contain one amino acid substitution or a combination thereof as defined in iii), and in some instances also said additional amino acids as defined herein above. Preferred combinations of said CDR sequences are listed in the following in the order of CDR-H1, CDR-H2, CDR-H3, CDR-11, CDR-L2, CDR-L3: 1878 to 1883; 1908 to 1913; 1984 to 1989; 1998 to 2003.

Also in accordance with the invention are CDR sequence combinations in the order of CDR-H1, CDR-H2, CDR-H3, CDR-L1, CDR-L2, CDR-L3 as defined in SEQ ID NOs: 126 to 131; 134 to 139; 142 to 147; 150 to 155; 158 to 163.

Also in accordance with the invention and more preferred are CDR sequence combinations as defined in SEQ ID NOs (listed in the order of CDR-H1, CDR-H2, CDR-H3, CDR-L1, CDR-L2, CDR-L3): 2006 to 2011; 2014 to 2019; 2022 to 2027; 2030 to 2035; 2038 to 2043; 20 to 25; 36 to 41; 44 to 49; 52 to 57; 60 to 65; 68 to 73; 76 to 81; 84 to 89; 92 to 97; 102 to 107; 110 to 115. Further examples are SEQ ID NOs: 282 to 287; 290 to 295; 298 to 303; 306 to 311; 314 to 319; 472 to 477; 480 to 485; 488 to 493; 496 to 501; 504 to 509; 650 to 655; 658 to 663; 666 to 671; 674 to 679; 682 to 687; 840 to 845; 848 to 853; 856 to 861; 864 to 869; 872 to 877; 1026 to 1031; 1034 to 1039; 1042 to 1047; 1050 to 1055; 1058 to 1063; 1216 to 1221; 1224 to 1229; 1232 to 1237; 1240 to 1245; 1248 to 1253; 1394 to 1399; 1402 to 1407; 1410 to 1415; 1418 to 1423; 1426 to 1431; 1584 to 1589; 1592 to 1597; 1600 to 1605; 1608 to 1613; 1616 to 1621; 1774 to 1779; 1782 to 1787; 1790 to 1795; 1798 to 1803; 1806 to 1811;

In accordance with the invention, the binding domain of the polypeptide or polypeptide construct of the invention that binds to an extracellular epitope of the human CD3ε chain comprises or consists of a VH region sequence as defined in the following SEQ ID NOs: 1856, 1860, 1862, 1864, 1872, 1884, 1894, 1896, 1898, 1900, 1902, 1904, 1906, 1914, 1916, 1918, 1920, 1922, 1924, 1926, 1928, 1930, 1932, 1934, 1936, 1938, 1940, 1942, 1944, 1946, 1948, 1950. Said sequences contain one amino acid substitution or a combination thereof as defined herein above, and in some instances also said additional amino acids as defined herein above. This is also true for the below embodiments characterizing by sequence ID number CD3epsilon binding domains that result from amino acid residue exchanges as specified herein.

In accordance with the invention, the binding domain of the polypeptide or polypeptide construct of the invention that binds to an extracellular epitope of the human CD3ε chain comprises or consists of a VL region sequence as defined in the following SEQ ID NOs: 1867, 1869, 1871, 1875, 1877, 1899, 1901, 1903, 1905, 1907, 1919, 1921, 1923, 1925, 1927, 1929, 1931, 1933, 1935, 1937, 1939, 1941, 1943, 1961, 1963, 1965, 1967, 1969, 1971, 1975, 1991, 1993, 1995, 1997, or 2005.

Preferred combinations of VH and VL region sequences of said binding domain of the polypeptide or polypeptide construct of the invention that binds to an extracellular epitope of the human CD3ε chain are defined in the following SEQ ID NOs: 1856 and 1857; 1860 and 1861; 1862 and 1863; 1864 and 1865; 1866 and 1867; 1868 and 1869; 1870 and 1871; 1872 and 1873; 1874 and 1875; 1876 and 1877; 1884 and 1885; 1894 and 1895; 1896 and 1897; 1898 and 1899; 1900 and 1901; 1902 and 1903; 1904 and 1905; 1906 and 1907; 1914 and 1915; 1916and 1917; 1918 and 1919; 1920and 1921; 1922 and 1923; 1924 and 1925; 1926 and 1927; 1928 and 1929; 1930 and 1931; 1932 and 1933; 1934 and 1935; 1936 and 1937; 1938 and 1939; 1940 and 1941; 1942 and 1943; 1944 and 1945; 1946 and 1947; 1948 and 1949; 1950 and 1951; 1960 and 1961; 1962 and 1963; 1964 and 1965; 1966 and 1967; 1968 and 1969; 1970 and 1971; 1974 and 1975; 1990 and 1991; 1992 and 1993; 1994 and 1995; 1996 and 1997; or 2004 and 2005.

Also in accordance with the invention are VH region sequences as defined in SEQ ID NO: 132, 140, 148, 156, and 164.

Also in accordance with the invention are VL region sequences as defined in SEQ ID NO: 133, 141, 149, 157, and 165.

Also in accordance with the invention are any combinations of the foregoing particularly preferred VH and VL region sequences. Preferred are the following VH and VL combinations as defined by SEQ ID NOs: 132 and 133; 140 and 141; 148 and 149; 156 and 157; and 164 and 165.

Also in accordance with the invention and more preferred are VH region sequences as defined in SEQ ID NO: 2012, 2020, 2028, 2036, 2044, 26, 34, 42, 50, 58, 66, 74, 82, 90, 98, 100, 108, and 116. Further examples are SEQ ID NOs: 288, 296, 304, 312, 320, 478, 486, 494, 502, 510, 656, 664, 672, 680, 688, 846, 854, 862, 870, 878, 1032, 1040, 1048, 1056, 1064, 1222, 1230, 1238, 1246, 1254, 1400, 1408, 1416, 1424, 1432, 1590, 1598, 1606, 1614, 1622, 1780, 1788, 1796, 1804, and 1812.

Also in accordance with the invention and more preferred are VL region sequences as defined in SEQ ID NO: 2013, 2021, 2029, 2037, 2045, 27, 35, 43, 51, 59, 67, 75, 83, 91, 99, 101, 109, and 117. Further examples are SEQ ID NOs: 289, 297, 305, 313, 321, 479, 487, 495, 503, 511, 657, 665, 673, 681, 689, 847, 855, 863, 871, 879, 1033, 1041, 1049, 1057, 1065, 1223, 1231, 1239, 1247, 1255, 1401, 1409, 1417, 1425, 1433, 1591, 1599, 1607, 1615, 1623, 1781, 1789, 1797, 1805, and 1813.

Also in accordance with the invention and preferred are any combinations of the foregoing particularly preferred VH and VL region sequences. Preferred are the following VH and VL combinations as defined by SEQ ID NOs: 2012 and 2013; 2020 and 2021; 2028 and 2029; 2036 and 2037; 2044 and 2045; 26 and 27; 34 and 35; 42 and 43; 50 and 51; 58 and 59; 66 and 67; 74 and 75; 82 and 83; 90 and 91; 98 and 99; 100 and 101; 108 and 109; and 116 and 117. Further examples are SEQ ID NOs: 288 and 289; 296 and 297; 304 and 305; 312 and 313; 320 and 321; 478 and 479; 486 and 487; 494 and 495; 502 and 503; 510 and 511; 656 and 657; 664 and 665; 672 and 673; 680 and 681; 688 and 689; 846 and 847; 854 855; 862 and 863; 870 and 871; 878 and 879; 1032 and 1033; 1040 and 1041; 1048 and 1049; 1056 and 1057; 1064 and 1065; 1222 and 1223; 1230 and 1231; 1238 and 1239; 1246 and 1247; 1254 and 1255; 1400 and 1401; 1408 and 1409; 1416 and 1417; 1424 and 1425; 1432 and 1433; 1590 and 1591; 1598 and 1599; 1606 and 1607; 1614 and 1615; 1622 and 1623; 1780 and 1781; 1788 and 1789; 1796 and 1797; 1804 and 1805; and 1812 and 1813.

In accordance with the invention, said binding domain of the polypeptide or polypeptide construct of the invention that binds to an extracellular epitope of the human CD3ε chain comprises or consists of a VH region sequence selected from SEQ ID NOs: 2012, 2020, 2028, 2036, 2044, 26, 34, 42, 50, 58, 66, 74, 82, 90, 98, 100, 108, 116, 288, 296, 304, 312, 320, 478, 486, 494, 502, 510, 656, 664, 672, 680, 688, 846, 854, 862, 870, 878, 1032, 1040, 1048, 1056, 1064, 1222, 1230, 1238, 1246, 1254, 1400, 1408, 1416, 1424, 1432, 1590, 1598, 1606, 1614, 1622, 1780, 1788, 1796, 1804, 1812, 1856, 1860, 1862, 1864, 1872, 1884, 1894, 1896, 1898, 1900, 1902, 1904, 1906, 1914, 1916, 1918, 1920, 1922, 1924, 1926, 1928, 1930, 1932, 1934, 1936, 1938, 1940, 1942, 1944, 1946, 1948, 1950, 132, 140, 148, 156, and 164; and/or a VL region sequence selected from SEQ ID NOs: 2013, 2021, 2029, 2037, 2045, 27, 35, 43, 51, 59, 67, 75, 83, 91, 99, 101, 109, and 117, 289, 297, 305, 313, 321, 479, 487, 495, 503, 511, 657, 665, 673, 681, 689, 847, 855, 863, 871, 879, 1033, 1041, 1049, 1057, 1065, 1223, 1231, 1239, 1247, 1255, 1401, 1409, 1417, 1425, 1433, 1591, 1599, 1607, 1615, 1623, 1781, 1789, 1797, 1805, 1813, 1867, 1869, 1871, 1875, 1877, 1899, 1901, 1903, 1905, 1907, 1919, 1921, 1923, 1925, 1927, 1929, 1931, 1933, 1935, 1937, 1939, 1941, 1943, 1961, 1963, 1965, 1967, 1969, 1971, 1975, 1991, 1993, 1995, 1997, 2005, 133, 141, 149, 157, and 165.

According to the invention, it is preferred that the polypeptide or polypeptide construct comprises: a binding domain binding to an extracellular epitope of the human CD3ε chain comprising or consisting of i) a VH region and ii) a VL region, wherein said VH and VL region of i) and ii) is selected from the VH and VL region combination as defined in SEQ ID NOs: 124 and 125, 172 and 173, 362 and 363, 552 and 553, 730 and 731, 928 and 929, 1106 and 1107, 1296 and 1297, 1474 and 1475, 1664 and 1665, and 1854 and 1855, wherein said one amino acid substitution or a combination thereof in said VH and/or VL region sequence results in a VH and/or a VL region sequence having the amino acid residue:
i. I at position 34, A at position 65, V at position 81, A at position 99, A at position 101, S at position 106 in the VH region sequence, I at position 20, K at position 40, S at position 69, and S at position 102 in the VL region sequence;
   M at position 45, A at position 65, L at position 81, A at position 101, T at position 106 in the VH region sequence, I at position 20, K at position 40, S at position 69 and S at position 102 in the VL region sequence;
   M at position 45, A at position 65, L at position 81, A at position 101, S at position 106, F at position 112 in the VH region sequence, I at position 20, I at position 38, K at position 40, E at position 69, S at position 102 and Y at position 93 in the VL region sequence;
   I at position 34, E at position 39, A at position 65, N at position 101, A at position 102,
   T at position 106, F at position 112 in the VH region sequence, K at position 40 and S at position 102 in the VL region sequence;
   I at position 34, E at position 64, A at position 65, A at position 99, A at position 101, T at position 106 in the VH region sequence, K at position 40, A at position 91 and S at position 102 in the VL region sequence;
   I at position 34, A at position 65, V at position 81, A at position 99, A at position 101, E at position 102, I at position 104, T at position 106 in the VH region sequence, I at position 20, K at position 40, S at position 69 and S at position 102 in the VL region sequence;
   E at position 64, A at position 65, G at position 68, V at position 81, A at position 101, S at position 106 in the VH region sequence, I at position 20, S at position 69 and S at position 102 in the VL region sequence;
   I at position 34, E at position 64, A at position 65, A at position 101, T at position 106, F at position 112 in the VH region sequence, K at position 40, A at position 91 and S at position 102 in the VL region sequence;
   I at position 34, E at position 64, A at position 65, A at position 101, S at position 106, F at position 112 in the VH region sequence, K at position 40, A at position 91 and S at position 102 in the VL region sequence;
   I at position 34, E at position 64, A at position 65, V at position 81, A at position 99, A at position 101, S at position 106 in the VH region sequence, I at position 20, K at position 40, S at position 69, S at position 102 and Y at position 93 in the VL region sequence;
   I at position 34, A at position 65, A at position 101, S at position 106, F at position 112 in the VH region sequence, K at position 40, A at position 91 and S at position 102 in the VL region sequence;
   M at position 45, E at position 64, A at position 65, L at position 81, A at position 101, T at position 106 in the VH region sequence, I at position 20, I at position 38, K at position 40, E at position 69 and S at position 102 in the VL region sequence;
   S at position 30, M at position 45, A at position 65, G at position 68, V at position 81, A at position 101, T at position 106, F at position 112 in the VH region sequence, K at position 40, S at position 69 and Y at position 93 in the VL region sequence; and
   S at position 30, M at position 45, E at position 64, A at position 65, G at position 68, V at position 81, A at position 101, S at position 106 in the VH region sequence, K at position 40 and S at position 69 in the VL region sequence;
ii. E at position 64, A at position 65, G at position 68, V at position 81, N at position 101, E at position 102, I at position 104, T at position 106 in the VH region sequence, I at position 20, S at position 69 and S at position 102 in the VL region sequence;
   A at position 65, G at position 68, V at position 81, N at position 101, E at position 102, I at position 104, S at position 106 in the VH region sequence, I at position 20, S at position 69 and S at position 102 in the VL region sequence;
   I at position 34, A at position 65, A at position 99, A at position 101, T at position 106 in the VH region sequence, K at position 40, A at position 91 and S at position 102 in the VL region sequence; and
   S at position 30, E at position 39, E at position 64, A at position 65, V at position 81, A at position 101, E at position 102, 1 at position 104, T at position 106 in the VH region sequence, M at position 20, K at position 40 and E at position 69 in the VL region sequence;
iii. A at position 101 in the VH region sequence;
iv. V at position 81, A at position 99_in the VH region sequence, I at position 20, K at position 40, S at position 69 and S at position 102 in the VL region sequence;
   I at position 34,_V at position 81 in the VH region sequence, I at position 20,_K at position 40 and S at position 102 in the VL region sequence;
   I at position 34 in the VH region sequence, I at position 20,_K at position 40_and S at position 102 in the VL region sequence;
   I at position 34,_E at position 39,_V at position 81 in the VH region sequence, I at position 20,_K at position 40 and S at position 102 in the VL region sequence;
v. I at position 34,_V at position 81 in the VH region sequence, and K at position 40 in the VL region sequence;
   M at position 45 in the VH region sequence, I at position 20,_K at position 40 and S at position 102 in the VH region sequence;
   I at position 34,_A at position 65,_A at position 101 and_S at position 106S in the VH region sequence;
   I at position 20,_K at position 40,_S at position 69 and_S at position 102 in the VL region sequence;
   F at position 34_and V at position 81 in the VH region sequence;
   M at position 45 in the VH region sequence, K at position 40 and_S at position 102 in the VL region sequence;
   D at position 39 in the VH region sequence, I at position 20,_K at position 40 and S at position 102 in the VL region sequence;
   I at position 34_and V at position 81 in the VH region sequence;
   E at position 39 in the VH region sequence, I at position 20,_K at position 40 and S at position 102 in the VL region sequence;
   M at position 45 in the VH region sequence and K at position 40 in the VL region sequence;
   I at position 34,_A at position 65,_V at position 81,_A at position 99,_A at position 101_and S at position 106 in the VH region sequence; or
vi. D at position 39 in the VH region sequence,_I at position 20,_R at position 40 and_S at position 102 in the VL region sequence;
   I at position 34 in the VH region sequence;
   K at position 40 in the VL region sequence;
   D at position 39_in the VH region sequence, K at position 40 and S at position 102 in the VL region sequence;
   I at position 20 and S at position 102 in the VH region sequence; K at position 39 in the VH region sequence;
   E at position 39 in the VH region sequence,_K at position 40_and S at position 102 in the VL region sequence;
   E at position 39 in the VH region sequence,_I at position 20,_R at position 40_and S at position 102 in the VL region sequence;
   D at position 39_in the VH region sequence,_R at position 40_and S at position 102 in the VL region sequence;
   V at position 81 in the VH region sequence;
   D at position 39 in the VH region sequence and_K at position 40 in the VL region sequence;
   F at position 112 in the VH region sequence;
   F at position 112 in the VH region sequence and_I at position 38 in the VL region sequence;
   E at position 39 in the VH region sequence and K at position 40 in the VL region sequence;
   V at position 34 in the VH region sequence.

The above recited amino acid residues are present at the given position(s) in the CD3epsilon binding domain according to the invention. In case one of the VH and/or VL region sequences that are to be changed (the VH region of i) and the VL region of ii)), i.e. the base VH and/or VL region sequence, already contain one of the above recited amino acid residues at a given position, it follows that these cannot changed since the respective amino acid residue is already present in the VH region and/or VL region of i) and/or ii).

More preferred is that the CD3epsilon binding domain comprises or consists of a combination of VH and VL region of i) and ii) selected from the group consisting of SEQ ID NOs: 124 and 125, 362 and 363, 730 and 731, 928 and 929, 1106 and 1107, 1296 and 1297, 1474 and 1475, 1664 and 1665, and 1854 and 1855. Even more preferred is that the CD3epsilon binding domain comprises or consists of a combination of VH and VL region of i) and ii) selected from the group consisting of SEQ ID NOs: 124 and 125, 730 and 731, 1664 and 1665, and 1854 and 1855. In a yet even more preferred embodiment, the CD3epsilon binding domain comprises or consists of a combination of VH and VL region of i) and ii) selected from the group consisting of SEQ ID NOs: 730 and 731, 1664 and 1665, and 1854 and 1855. Most preferred is that said CD3epsilon binding domain comprises or consists of a combination of VH and VL region of i) and ii) of SEQ ID NOs: 1854 and 1855, respectively.

As evident from the example section, the above recited amino acid residues at the given position(s) have a favorable effect on an increase of the temperature stability that is measured by the well know and also herein described method of DSF (Differential Scanning Fluorimetry; cf. e.g., Wen et al., “Nano differential scanning fluorimetry for comparability studies of therapeutic proteins”, Analytical Biochemistry, Volume 593, 2020, 113581, ISSN 0003-2697; Dart, M. L., et al. (2018). “Homogeneous Assay for Target Engagement Utilizing Bioluminescent Thermal Shift”. ACS medicinal chemistry letters, 9(6), 546-551) when introduced in the above recited VH and VL sequences of i) and ii). More specifically, the amino acid residues at the given positions recited in sections i. and iv. exhibit an increase in temperature stability of 6° C. or more as compared to the value measured for the unmodified sequence, i.e. the respective base VH and VL sequence. Section ii. and v. exhibit an increase in temperature stability of 3° C. or more as compared to the value measured for the unmodified sequence, i.e. the respective base VH and VL sequence, whereas sections iii. and vi. exhibit an increase in temperature stability of 1° C. or more as compared to the value measured for the unmodified sequence, i.e. the respective base VH and VL sequence. Preferably, the amino acid residues at the given position(s) in section i. ii., iv., and/or v. are present in the CD3epsilon binding domain according to this embodiment. More preferred is that the amino acid residues at the given position(s) in section i. and/or iv. are present in the CD3epsilon binding domain according to the invention. This preference also applies to the preferred embodiments recited in the following. As outlined herein, it is also preferred that the VH and VL region is linked by a linker, preferably a peptide linker most preferred a G₄S or a G₄Q linker, preferably said G₄S or a G₄Q linker is repeated three times, i.e. it is a (G₄S)₃ or a (G₄Q)₃ linker.

According to the invention, it relates to a polypeptide or polypeptide construct comprising: a binding domain binding to an extracellular epitope of the human CD3ε chain comprising or consisting of a VH region and a VL region, wherein
a) the VH region comprises or consist of the sequence as defined in SEQ ID NO: 1854 and the VL region comprises or consists of the sequence as defined in SEQ ID NO: 1855; and wherein the VH and/or VL region sequence comprise the amino acid substitution(s) selected from:
   i. M34I, S65A, A81V, V99A, H101A, N106S in the VH region sequence (resulting VH sequence as defined in SEQ ID NO: 2012), L20I, Q40K, L69S, and G102S in the VL region sequence (resulting VL sequence as defined in SEQ ID NO: 2013);
      L45M, S65A, A81L, H101A, N106T in the VH region sequence (SEQ ID NO: 2036), L20I, Q40K, L69S, and G102S in the VL region sequence (SEQ ID NO: 2037);
      L45M, S65A, A81L, H101A, N106S, W112F in the VH region sequence (SEQ ID NO: 2028), L20I, V381, Q40K, L69E, G102S and W93Y in the VL region sequence (SEQ ID NO: 2029);
      M34I, Q39E, S65A, H101N, G102A, N106T, W112F in the VH region sequence (SEQ ID NO: 2044), Q40K and G102S in the VL region sequence (SEQ ID NO: 2045);
      M34I, D64E, S65A, V99A, H101A, N106T in the VH region sequence (SEQ ID NO: 34), Q40K, V91A and G102S in the VL region sequence (SEQ ID NO: 35);
      M34I, S65A, A81V, V99A, H101A, G102E, F104I, N106T in the VH region sequence (SEQ ID NO: 90), L20I, Q40K, L69S, and G102S in the VL region sequence (SEQ ID NO: 91);
      D64E, S65A, D68G, A81V, H101A, N106S in the VH region sequence (SEQ ID NO: 100), L20I, L69S, and G102S in the VL region sequence (SEQ ID NO: 101);
      M34I, D64E, S65A, H101A, N106T, W112F in the VH region sequence (SEQ ID NO: 58), Q40K, V91A and G102S in the VL region sequence (SEQ ID NO: 59);
      M34I, D64E, S65A, H101A, N106S, W112F in the VH region sequence (SEQ ID NO: 50), Q40K, V91A and G102S in the VL region sequence (SEQ ID NO: 51);
      M34I, D64E, S65A, A81V, V99A, H101A, N106S in the VH region sequence (SEQ ID NO: 98), L20I, Q40K, L69S, G102S and W93Y in the VL region sequence (SEQ ID NO: 99);
      M34I, S65A, H101A, N106S, W112F in the VH region sequence (SEQ ID NO: 42), Q40K, V91A and G102S in the VL region sequence (SEQ ID NO: 43);
      L45M, D64E, S65A, A81L, H101A, N106T in the VH region sequence (SEQ ID NO: 116), L20I, V381, Q40K, L69E and G102S in the VL region sequence (SEQ ID NO: 117);
      N30S, L45M, S65A, D68G, A81V, H101A, N106T, W112F in the VH region sequence (SEQ ID NO: 82), Q40K, L69S and W93Y in the VL region sequence (SEQ ID NO: 83); and
      N30S, L45M, D64E, S65A, D68G, A81V, H101A, N106S in the VH region sequence (SEQ ID NO: 74), Q40K and L69S in the VL region sequence (SEQ ID NO: 75);
   ii. D64E, S65A, D68G, A81V, H101N, G102E, F104I, N106T in the VH region sequence (SEQ ID NO: 2020), L20I, L69S, and G102S in the VL region sequence (SEQ ID NO: 2021);
      S65A, D68G, A81V, H101N, G102E, F104I, N106S in the VH region sequence (SEQ ID NO: 108), L20I, L69S, and G102S in the VL region sequence (SEQ ID NO: 109);
      M34I, S65A, V99A, H101A, N106T in the VH region sequence (SEQ ID NO: 26), Q40K, V91A and G102S in the VL region sequence (SEQ ID NO: 27); and
      N30S, Q39E, D64E, S65A, A81V, H101A, G102E, F104I, N106T in the VH region sequence (SEQ ID NO: 66), L20M, Q40K and L69E in the VL region sequence (SEQ ID NO: 67);
   iii. H101A in the VH region sequence (SEQ ID NO: 2560);
   iv. A81V, V99A_in the VH region sequence, L20I, Q40K, L69S and G102S in the VL region sequence;
      M34I,_A81V in the VH region sequence (SEQ ID NO: 1898),_L20I,_Q40K and_G102S in the VL region sequence (SEQ ID NO: 1899);
      M34I in the VH region sequence (SEQ ID NO: 1904), L20I,_Q40K_and G102S in the VL region sequence (1905);
      M34I,_Q39E,_A81V in the VH region sequence (SEQ ID NO: 1906), L20I,_Q40K and_G102S in the VL region sequence (SEQ ID NO: 1907);
   v. M34I,_A81V in the VH region sequence (SEQ ID NO: 1900), and Q40K in the VL region sequence (SEQ ID NO: 1901);
      L45M in the VH region sequence (SEQ ID NO: 1866),_L20I,_Q40K and_G102S in the VH region sequence (SEQ ID NO: 1867);
      M34I,_S65A,_H101A and_N106S in the VH region sequence; L20I,_Q40K,_L69S and_G102S in the VL region sequence; M34F_and A81V in the VH region sequence (SEQ ID NO: 1884);
      L45M in the VH region sequence (SEQ ID NO: 1870), Q40K and_G102S in the VL region sequence (SEQ ID NO: 1871); Q39D in the VH region sequence (SEQ ID NO: 1918), L20I,_Q40K and G102S in the VL region sequence (SEQ ID NO: 1919);
      M34I_and A81V in the VH region sequence (SEQ ID NO: 1869); Q39E in the VH region sequence (SEQ ID NO:_1932), L20I,_Q40K and_G102S in the VL region sequence (SEQ ID NO: 1933);
      L45M in the VH region sequence (SEQ ID NO:_1868)_and_Q40K in the VI region sequence (SEQ ID NO: 1869);
      M34I,_S65A,_A81V,_V99A,_H101A_and N106S in the VH region sequence; or
   vi. Q39D in the VH region sequence (SEQ ID NO: 1928),_L20I,_Q40R and_G102S in the VL region sequence (SEQ ID NO: 1929);
      M34I in the VH region sequence (SEQ ID NO: 1892); Q40K in the VL region sequence (SEQ ID NO: 1974); Q39D_in the VH region sequence (SEQ ID NO: 1922), Q40K and_G102S in the VL region sequence (SEQ ID NO: 1923);
      L20I and_G102S in the VH region sequence;
      Q39K in the VH region sequence (SEQ ID NO: 1944);
      Q39E in the VH regions sequence (SEQ ID NO: 1938),_Q40K_and G102S in the VL region sequence (SEQ ID NO: 1939);
      Q39E in the VH region sequence (SEQ ID NO: 1934),_L20I,_Q40R_and G102S in the VL region sequence (SEQ ID NO: 1935);
      Q39D_in the VH region sequence (SEQ ID NO: 1926),_Q40R_and G102S in the VL region sequence (SEQ ID NO: 1926);
      A81V in the VH region sequence (SEQ ID NO: 1862);
      Q39D in the VH region sequence (SEQ ID NO: 1920) and_Q40K in the VL region sequence (SEQ ID NO: 1921);
      W112F in the VH region sequence (SEQ ID NO: 1958);
      W112F in the VH region sequence (SEQ ID NO: 1960) and_V381 in the VL region sequence (SEQ ID NO: 1961);
      Q39E in the VH region sequence (SEQ ID NO: 1936)_and_Q40K in the VL region sequence (SEQ ID NO: 1937);
      M34V in the VH region sequence (SEQ ID NO: 1914);
b) the VH region comprises or consist of the sequence as defined in SEQ ID NO: 730 and the VL region comprises or consists of the sequence as defined in SEQ ID NO: 731; and wherein the VH and/or VL region sequence comprise the amino acid substitution(s) selected from:
   i. M34I, S65A, A81V, V99A, H101A, N106S in the VH region sequence (resulting VH sequence as defined in SEQ ID NO: 846), L20I, Q40K, L69S, and G102S in the VL region sequence (resulting VL sequence as defined in SEQ ID NO: 847);
      L45M, S65A, A81L, H101A, N106T in the VH region sequence (SEQ ID NO: 870), L20I, Q40K, L69S, and G102S in the VL region sequence (SEQ ID NO: 871);
      L45M, S65A, A81L, H101A, N106S, W112F in the VH region sequence (SEQ ID NO: 862), L20I, V381, Q40K, L69E, G102S and W93Y in the VL region sequence (SEQ ID NO: 863);
      M34I, Q39E, S65A, H101N, G102A, N106T, W112F in the VH region sequence (SEQ ID NO: 878), Q40K and G102S in the VL region sequence (SEQ ID NO: 879);
      M34I, D64E, S65A, V99A, H101A, N106T in the VH region sequence, Q40K, V91A and G102S in the VL region sequence;
      M34I, S65A, A81V, V99A, H101A, G102E, F104I, N106T in the VH region sequence, L20I, Q40K, L69S, and G102S in the VL region sequence; D64E, S65A, A81V, H101A, N106S in the VH region sequence, L20I, L69S, and G102S in the VL region sequence;
      M34I, D64E, S65A, H101A, N106T, W112F in the VH region sequence, Q40K, V91A and G102S in the VL region sequence;
      M34I, D64E, S65A, H101A, N106S, W112F in the VH region sequence, Q40K, V91A and G102S in the VL region sequence;
      M34I, D64E, S65A, A81V, V99A, H101A, N106S in the VH region sequence, L20I, Q40K, L69S, G102S and W93Y in the VL region sequence;
      M34I, S65A, H101A, N106S, W112F in the VH region sequence, Q40K, V91A and G102S in the VL region sequence;
      L45M, D64E, S65A, A81L, H101A, N106T in the VH region sequence, L20I, V381, Q40K, L69E and G102S in the VL region sequence;
      N30S, L45M, S65A, A81V, H101A, N106T, W112F in the VH region sequence, Q40K, L69S and W93Y in the VL region sequence; and
      N30S, L45M, D64E, S65A, A81V, H101A, N106S in the VH region sequence, Q40K and L69S in the VL region sequence;
   ii. D64E, S65A, A81V, H101N, G102E, F104I, N106T in the VH region sequence (SEQ ID NO: 854), L20I, L69S, and G102S in the VL region sequence (SEQ ID NO: 855);
      S65A, A81V, H101N, G102E, F104I, N106S in the VH region sequence, L20I, L69S, and G102S in the VL region sequence;
      M34I, S65A, V99A, H101A, N106T in the VH region sequence, Q40K, V91A and G102S in the VL region sequence; and
      N30S, Q39E, D64E, S65A, A81V, H101A, G102E, F104I, N106T in the VH region sequence, L20M, Q40K and L69E in the VL region sequence;
   iii. H101A in the VH region sequence;
   iv. A81V, V99A_in the VH region sequence, L20I, Q40K, L69S and G102S in the VL region sequence;
      M34I,_A81V in the VH region sequence (SEQ ID NO: 774),_L20I,_Q40K and_G102S in the VL region sequence (SEQ ID NO: 775);
      M34I in the VH region sequence (SEQ ID NO: 780), L20I,_Q40K_and G102S in the VL region sequence (781);
      M34I,_Q39E,_A81V in the VH region sequence (SEQ ID NO: 782), L20I,_Q40K and_G102S in the VL region sequence (SEQ ID NO: 783);
   v. M34I,_A81V in the VH region sequence (SEQ ID NO: 776), and Q40K in the VL region sequence (SEQ ID NO: 777);
      L45M in the VH region sequence (SEQ ID NO: 742),_L20I,_Q40K and_G102S in the VH region sequence (SEQ ID NO: 743);
      M34I,_S65A,_H101A and_N106S in the VH region sequence;
      L20I,_Q40K,_L69S and_G102S in the VL region sequence;
      M34F_and A81V in the VH region sequence (SEQ ID NO: 760);
      L45M in the VH region sequence (SEQ ID NO: 746), Q40K and_G102S in the VL region sequence (SEQ ID NO: 747);
      Q39D in the VH region sequence (SEQ ID NO: 794), L20I,_Q40K and G102S in the VL region sequence (SEQ ID NO: 795);
      M34I_and A81V in the VH region sequence (SEQ ID NO: 772);
      Q39E in the VH region sequence (SEQ ID NO:_808), L20I,_Q40K and_G102S in the VL region sequence (SEQ ID NO: 809);
      L45M in the VH region sequence (SEQ ID NO:_744)_and_Q40K in the VI region sequence (SEQ ID NO: 745);
      M34I,_S65A,_A81V,_V99A,_H101A_and N106S in the VH region sequence; or
   vi. Q39D in the VH region sequence (SEQ ID NO: 796),_L20I,_Q40R and_G102S in the VL region sequence (SEQ ID NO: 797);
      M34I in the VH region sequence (SEQ ID NO: 768);
      Q40K in the VL region sequence (SEQ ID NO: 891);
      Q39D_in the VH region sequence (SEQ ID NO: 800), Q40K and_G102S in the VL region sequence (SEQ ID NO: 801);
      L20I and_G102S in the VH region sequence;
      Q39K in the VH region sequence (SEQ ID NO: 820);
      Q39E in the VH region sequence (SEQ ID NO: 814),_Q40K_and G102S in the VL region sequence (SEQ ID NO: 815);
      Q39E in the VH region sequence (SEQ ID NO: 810),_L20I,_Q40R_and G102S in the VL region sequence (SEQ ID NO: 811);
      Q39D_in the VH region sequence (SEQ ID NO: 804),_Q40R_and G102S in the VL region sequence (SEQ ID NO: 805);
      A81V in the VH region sequence (SEQ ID NO: 738);
      Q39D in the VH region sequence (SEQ ID NO: 798) and_Q40K in the VL region sequence (SEQ ID NO: 799);
      W112F in the VH region sequence (SEQ ID NO: 834);
      W112F in the VH region sequence (SEQ ID NO: 836) and_V381 in the VL region sequence (SEQ ID NO: 837);
      Q39E in the VH region sequence (SEQ ID NO: 812)_and_Q40K in the VL region sequence (SEQ ID NO: 813);
      M34V in the VH region sequence (SEQ ID NO: 790)
c) the VH region comprises or consist of the sequence as defined in SEQ ID NO: 1664 and the VL region comprises or consists of the sequence as defined in SEQ ID NO: 1665; and wherein the VH and/or VL region sequence comprise the amino acid substitution(s) selected from:
   i. M34I, S65A, A81V, V99A, H101A, N106S in the VH region sequence (resulting VH sequence as defined in SEQ ID NO: 1780), L20I, Q40K, L69S, and G102S in the VL region sequence (resulting VL sequence as defined in SEQ ID NO: 1781);
      L45M, S65A, A81L, H101A, N106T in the VH region sequence (SEQ ID NO: 1804), L20I, Q40K, L69S, and G102S in the VL region sequence (SEQ ID NO: 1805);
      L45M, S65A, A81L, H101A, N106S, W112F in the VH region sequence (SEQ ID NO: 1796), L20I, V381, Q40K, L69E, G102S and W93Y in the VL region sequence (SEQ ID NO: 1797);
      M34I, Q39E, S65A, H101N, G102A, N106T, W112F in the VH region sequence (SEQ ID NO: 1812), Q40K and G102S in the VL region sequence (SEQ ID NO: 1813);
      M34I, D64E, S65A, V99A, H101A, N106T in the VH region sequence, Q40K and G102S in the VL region sequence;
      M34I, S65A, A81V, V99A, H101A, G102E, F104I, N106T in the VH region sequence, L20I, Q40K, L69S, and G102S in the VL region sequence; D64E, S65A, D68G, A81V, H101A, N106S in the VH region sequence, L20I, L69S, and G102S in the VL region sequence;
      M34I, D64E, S65A, H101A, N106T, W112F in the VH region sequence, Q40K and G102S in the VL region sequence;
      M34I, D64E, S65A, H101A, N106S, W112F in the VH region sequence, Q40K and G102S in the VL region sequence;
      M34I, D64E, S65A, A81V, V99A, H101A, N106S in the VH region sequence, L20I, Q40K, L69S, G102S and W93Y in the VL region sequence;
      M34I, S65A, H101A, N106S, W112F in the VH region sequence, Q40K and G102S in the VL region sequence;
      L45M, D64E, S65A, A81L, H101A, N106T in the VH region sequence, L20I, V381, Q40K, L69E and G102S in the VL region sequence;
      N30S, L45M, S65A, D68G, A81V, H101A, N106T, W112F in the VH region sequence, Q40K, L69S and W93Y in the VL region sequence; and
      N30S, L45M, D64E, S65A, D68G, A81V, H101A, N106S in the VH region sequence, Q40K and L69S in the VL region sequence;
   ii. D64E, S65A, D68G, A81V, H101N, G102E, F104I, N106T in the VH region sequence (SEQ ID NO: 1788), L20I, L69S, and G102S in the VL region sequence (SEQ ID NO: 1789);
      S65A, D68G, A81V, H101N, G102E, F104I, N106S in the VH region sequence, L20I, L69S, and G102S in the VL region sequence;
      M34I, S65A, V99A, H101A, N106T in the VH region sequence, Q40K and G102S in the VL region sequence; and
      N30S, Q39E, D64E, S65A, A81V, H101A, G102E, F104I, N106T in the VH region sequence, L20M, Q40K and L69E in the VL region sequence;
   iii. H101A in the VH region sequence;
   iv. A81V, V99A_in the VH region sequence, L20I, Q40K, L69S and G102S in the VL region sequence;
      M34I,_A81V in the VH region sequence (SEQ ID NO: 1708),_L20I,_Q40K and_G102S in the VL region sequence (SEQ ID NO: 1709);
      M34I in the VH region sequence (SEQ ID NO: 1714), L20I,_Q40K_and G102S in the VL region sequence (1715);
      M34I,_Q39E,_A81V in the VH region sequence (SEQ ID NO: 1716), L20I,_Q40K and_G102S in the VL region sequence (SEQ ID NO: 1717);
   v. M34I,_A81V in the VH region sequence (SEQ ID NO: 1710), and Q40K in the VL region sequence (SEQ ID NO: 1711);
      L45M in the VH region sequence (SEQ ID NO: 1676),_L20I,_Q40K and_G102S in the VH region sequence (SEQ ID NO: 1677);
      M34I,_S65A,_H101A and_N106S in the VH region sequence;
      L20I,_Q40K,_L69S and_G102S in the VL region sequence;
      M34F_and A81V in the VH region sequence (SEQ ID NO: 1694);
      L45M in the VH region sequence (SEQ ID NO: 1680), Q40K and_G102S in the VL region sequence (SEQ ID NO: 1681);
      Q39D in the VH region sequence (SEQ ID NO: 1728), L20I,_Q40K and G102S in the VL region sequence (SEQ ID NO: 1729);
      M34I_and A81V in the VH region sequence (SEQ ID NO: 1706);
      Q39E in the VH region sequence (SEQ ID NO:_1742), L20I,_Q40K and_G102S in the VL region sequence (SEQ ID NO: 1743);
      L45M in the VH region sequence (SEQ ID NO:_1678)_and_Q40K in the VI region sequence (SEQ ID NO: 1679);
      M34I,_S65A,_A81V,_V99A,_H101A_and N106S in the VH region sequence; or
   vi. Q39D in the VH region sequence (SEQ ID NO: 1730),_L20I,_Q40R and_G102S in the VL region sequence (SEQ ID NO: 1731);
      M34I in the VH region sequence (SEQ ID NO: 1702);
      Q40K in the VL region sequence (SEQ ID NO: 1755);
      Q39D_in the VH region sequence (SEQ ID NO: 1734), Q40K and_G102S in the VL region sequence (SEQ ID NO: 1735);
      L20I and_G102S in the VH region sequence;
      Q39K in the VH region sequence (SEQ ID NO: 1754);
      Q39E in the VH regions sequence (SEQ ID NO: 1748),_Q40K_and G102S in the VL region sequence (SEQ ID NO: 1749);
      Q39E in the VH region sequence (SEQ ID NO: 1744),_L20I,_Q40R_and G102S in the VL region sequence (SEQ ID NO: 1745);
      Q39D_in the VH region sequence (SEQ ID NO: 1738),_Q40R_and G102S in the VL region sequence (SEQ ID NO: 1739);
      A81V in the VH region sequence (SEQ ID NO: 1672);
      Q39D in the VH region sequence (SEQ ID NO: 1732) and_Q40K in the VL region sequence (SEQ ID NO: 1733);
      W112F in the VH region sequence (SEQ ID NO: 1768);
      W112F in the VH region sequence (SEQ ID NO: 1770) and_V381 in the VL region sequence (SEQ ID NO: 1771);
      Q39E in the VH region sequence (SEQ ID NO: 1746)_and_Q40K in the VL region sequence (SEQ ID NO: 1747);
      M34V in the VH region sequence (SEQ ID NO: 1724); or
d) the VH region comprises or consist of the sequence as defined in SEQ ID NO: 124 and the VL region comprises or consists of the sequence as defined in SEQ ID NO: 125; and wherein the VH and/or VL region sequence comprise the amino acid substitution(s) selected from:
   i. Q65A, A81V, V99A, H101A, N106S in the VH region sequence (resulting VH sequence as defined in SEQ ID NO: 132), L20I, Q40K, L69S, and G102S in the VL region sequence (resulting VL sequence as defined in SEQ ID NO: 133);
      L45M, Q65A, A81L, H101A, N106T in the VH region sequence (SEQ ID NO: 156), L20I, Q40K, L69S, and G102S in the VL region sequence (SEQ ID NO: 157);
      L45M, Q65A, A81L, H101A, N106S, W112F in the VH region sequence (SEQ ID NO: 148), L20I, V381, Q40K, L69E, G102S and W93Y in the VL region sequence (SEQ ID NO: 149);
      Q39E, Q65A, H101N, N106T, W112F in the VH region sequence (SEQ ID NO: 164), Q40K and G102S in the VL region sequence (SEQ ID NO: 165);
      D64E, Q65A, V99A, H101A, N106T in the VH region sequence, Q40K, T91A and G102S in the VL region sequence;
      Q65A, A81V, V99A, H101A, A102E, F104I, N106T in the VH region sequence, L20I, Q40K, L69S, and G102S in the VL region sequence;
      D64E, Q65A, D68G, A81V, H101A, N106S in the VH region sequence, L20I, L69S, and G102S in the VL region sequence;
      D64E, Q65A, H101A, N106T, W112F in the VH region sequence, Q40K and G102S in the VL region sequence;
      D64E, Q65A, H101A, N106S, W112F in the VH region sequence, Q40K and G102S in the VL region sequence;
      D64E, Q65A, A81V, V99A, H101A, N106S in the VH region sequence, L20I, Q40K, L69S, G102S and W93Y in the VL region sequence;
      Q65A, H101A, N106S, W112F in the VH region sequence, Q40K and G102S in the VL region sequence;
      L45M, D64E, Q65A, A81L, H101A, N106T in the VH region sequence, L20I, V381, Q40K, L69E and G102S in the VL region sequence;
      N30S, L45M, Q65A, D68G, A81V, H101A, N106T, W112F in the VH region sequence, Q40K, L69S and W93Y in the VL region sequence; and
      N30S, L45M, D64E, Q65A, D68G, A81V, H101A, N106S in the VH region sequence, Q40K and L69S in the VL region sequence;
   ii. D64E, Q65A, D68G, A81V, H101N, A102E, F104I, N106T in the VH region sequence (SEQ ID NO: 140), L20I, L69S, and G102S in the VL region sequence (SEQ ID NO: 141);
      Q65A, D68G, A81V, H101N, A102E, F104I, N106S in the VH region sequence, L20I, L69S, and G102S in the VL region sequence;
      Q65A, V99A, H101A, N106T in the VH region sequence, Q40K and G102S in the VL region sequence; and
      N30S, Q39E, D64E, Q65A, A81V, H101A, A102E, F104I, N106T in the VH region sequence, L20M, Q40K and L69E in the VL region sequence;
   iii. H101A in the VH region sequence;
   iv. A81V, V99A_in the VH region sequence, L20I, Q40K, L69S and G102S in the VL region sequence;
      A81V in the VH region sequence,_L20I,_Q40K and_G102S in the VL region sequence;
      L20I,_Q40K_and G102S in the VL region sequence;
      Q39E,_A81V in the VH region sequence, L20I,_Q40K and_G102S in the VL region sequence;
   v. A81V in the VH region sequence, and Q40K in the VL region sequence;
      L45M in the VH region sequence,_L20I,_Q40K and_G102S in the VH region sequence;
      Q65A,_H101A and_N106S in the VH region sequence;
      L20I,_Q40K,_L69S and_G102S in the VL region sequence; 134F_and A81V in the VH region sequence;
      L45M in the VH region sequence, Q40K and_G102S in the VL region sequence;
      Q39D in the VH region sequence, L20I,_Q40K and G102S in the VL region sequence;
      Q39E in the VH region sequence, L20I,_Q40K and_G102S in the VL region sequence;
      L45M in the VH region sequence_and_Q40K in the VL region sequence; Q65A,_A81V,_V99A,_H101A_and N106S in the VH region sequence; or
   vi. Q39D in the VH region sequence,_L20I,_Q40R and_G102S in the VL region sequence;
      Q40K in the VL region sequence;
      Q39D_in the VH region sequence, Q40K and_G102S in the VL region sequence;
      L20I and_G102S in the VH region sequence;
      Q39K in the VH region sequence;
      Q39E in the VH region sequence,_Q40K_and G102S in the VL region sequence;
      Q39E in the VH region sequence,_L20I,_Q40R_and G102S in the VL region sequence;
      Q39D_in the VH region sequence,_Q40R_and G102S in the VL region sequence);
      A81V in the VH region sequence;
      Q39D in the VH region sequence and_Q40K in the VL region sequence;
      W112F in the VH region sequence;
      W112F in the VH region sequence and_V381 in the VL region sequence;
      Q39E in the VH region sequence_and_Q40K in the VL region sequence;
      M34V in the VH region sequence.
         The alternatives a) to c) are preferred, more preferred is the alternative a) as recited above as CD3epsilon binding domain of the polypeptide or polypeptide construct of the invention. The above recited preferred linkers are also preferred for this embodiment, i.e (G₄S)₃ or G₄Q)3.

In accordance with the invention, the polypeptide or polypeptide construct the invention may have linkers, half-life extending peptides, and other structural moieties as disclosed in SEQ ID NOs: 1 to 19 and 2551. Details on nature and functions of these structures are found in the sequence table disclosed herein.

The invention provides an embodiment wherein the polypeptide construct is in a format selected from the group consisting of scFv, and to the extent that a further binding domain is present (scFv)2, diabodies and oligomers of any of the aforementioned formats. The term “is in a format” does not exclude that the construct can be further modified, e.g. by attachment or fusion to other moieties, as described herein. According to one embodiment of the polypeptide construct of the present invention, the domains comprising the herein described paratopes are in the format of an scFv. In an scFv, the VH region and the and VL region are arranged in the order VH-VL or VL-VH (from N- to C-terminus). It is envisaged that the VH and the VL regions of the domain(s) are connected via a linker, preferably a peptide linker. In a preferred embodiment, the peptide linker is a G₄S, or G₄Q linker or repetitions thereof, such as preferably (G₄S)3 (i.e. three repetitions of G₄S), or (G₄Q)3 (i.e. three repetitions of G₄Q). According to one embodiment of the binding domain(s) comprising the herein described VH and the VL regions, the VH-region is positioned N-terminally of the linker, and the VL-region is positioned C-terminally of the linker. In other words, in one embodiment of the domains comprising the herein described paratopes, the scFv comprises from the N-terminus to the C-terminus: VH-linker-VL. It is furthermore envisaged that binding domain(s) (comprising the herein described paratopes) of the construct, in case the polypeptide or polypeptide construct comprises at least one further binding domain in addition to said CD3 epsilon binding domain, are connected via a linker, preferably a peptide linker. The construct may e.g. comprise the domains in the order (from N-terminus to C-terminus) CD3 binding domain-linker-further binding domain. The inverse order (further binding domain(s)-linker-CD3 binding domain) is also possible and is the preferred orientation.

The linkers are preferably peptide linkers, more preferably short peptide linkers. In accordance with the present invention, a “peptide linker” comprises an amino acid sequence which connects the amino acid sequences of one domain with another (variable and/or binding) domain (e.g. a variable domain or a binding domain) of the construct. An essential technical feature of such peptide linker is that it does not comprise any polymerization activity. Among the suitable peptide linkers are those described in U.S. Pat. Nos. 4,751,180 and 4,935,233 or WO 88/09344. The peptide linkers can also be used to attach other domains or modules or regions (such as half-life extending domains) to the construct of the invention. Examples of useful peptide linkers are shown in SEQ ID NOs: 1 to 11 and 2551. In the present context, a “short” linker has between 2 and 50 amino acids, preferably between 3 and 35, between 4 and 30, between 5 and 25, between 6 and 20, or between 6 and 17 amino acids. The linker between two variable regions of one binding domain may have a different length (e.g. may be longer) than the linker between the two binding domains. For example, the linker between two variable regions of one binding domain may have a length between 7 and 15 amino acids, preferably between 9 and 13, and the linker between the two binding domains may have a length between 3 and 10 amino acids, preferably between 4 and 8. It is further envisaged that the peptide linkers are glycine/serine linkers, such as those depicted in SEQ ID NOs: 1 to 11 and 2551. Most of the amino acids in glycine/serine linkers are selected from glycine and serine.

If a linker is used, this linker is preferably of a length and sequence to ensure that each of the first and second domains can, independently from one another, retain their differential binding specificities. For peptide linkers which connect the at least two binding domains (or the two variable regions forming one binding domain) in the construct, those peptide linkers are envisaged which comprise only a few amino acid residues, e.g. 12 amino acid residues or less. Thus, peptide linkers of 12, 11, 10, 9, 8, 7, 6 or 5 amino acid residues are preferred. An envisaged peptide linker with less than 5 amino acids comprises 4, 3, 2 or one amino acid(s), wherein Gly-rich linkers are preferred. A “single amino acid” linker in the context of said “peptide linker” is Gly. Another embodiment of a peptide linker is characterized by the amino acid sequence Gly-Gly-Gly-Gly-Ser, i.e. Gly4Ser (SEQ ID NO: 15), or polymers thereof, i.e. (Gly4Ser)x, where x is an integer of 1 or greater (e.g. 2 or 3), for example SEQ ID NOs: 1 and 2. Preferred linkers are depicted in SEQ ID NOs: 2, 4, 5, 6, 8, 10, 11, and 2551. Another preferred linker comprises or consists of (Gly4Ser)6. The characteristics of said peptide linkers are known in the art and are described e.g. in Dall'Acqua et al. (Biochem. (1998) 37, 9266-9273), Cheadle et al. (Mol Immunol (1992) 29, 21-30) and Raag and Whitlow (FASEB (1995) 9(1), 73-80). Peptide linkers which do not promote any secondary structures are preferred. The linkage of said domains to each other can be provided, e.g., by genetic engineering. Methods for preparing fused and operatively linked bispecific single chain constructs and expressing them in mammalian cells or bacteria are well-known in the art (e.g. WO 99/54440 or Sambrook et al., Molecular Cloning: A Laboratory Manual, Cold Spring Harbor Laboratory Press, Cold Spring Harbor, New York, 2001). More preferred, said peptide linker is a G₄S, or G₄Q linker or repetitions thereof, such as (G₄S)3 or (G₄Q)3.

According to one embodiment of the invention, the polypeptide construct of the invention is a “single chain construct” or “single chain polypeptide”. In the case of a further binding domain, it is also envisaged that either the CD3 binding or the further (also termed “second”) or both binding domains may be in the format of a “single chain Fv” (scFv). Although the two domains of the Fv fragment, VL and VH, are coded for by separate genes, they can be joined, using recombinant methods, by an artificial linker—as described hereinbefore—that enables them to be made as a single protein chain in which the VL and VH regions pair to form a monovalent molecule; see e.g., Huston et al. (1988) Proc. Natl. Acad. Sci USA 85:5879-5883). These antibody fragments are obtained using conventional techniques known to those with skill in the art, and the fragments are evaluated for function in the same manner as are full-length antibodies or IgGs. A single-chain variable fragment (scFv) is hence a fusion protein of the variable region of the heavy chain (VH) and of the light chain (VL) of immunoglobulins, usually connected with a short linker peptide. The linker is usually rich in glycine for flexibility, as well as serine or also threonine for solubility, and can either connect the N-terminus of the VH with the C-terminus of the VL, or vice versa. This protein retains the specificity of the original immunoglobulin, despite removal of the constant regions and introduction of the linker.

Bispecific single chain molecules are known in the art and are described in WO 99/54440, Mack, J. Immunol. (1997), 158, 3965-3970, Mack, PNAS, (1995), 92, 7021-7025, Kufer, Cancer Immunol. Immunother., (1997), 45, 193-197, Löffler, Blood, (2000), 95, 6, 2098-2103, Brühl, Immunol., (2001), 166, 2420-2426, Kipriyanov, J. Mol. Biol., (1999), 293, 41-56. Techniques described for producing single chain constructs (see, inter alia, U.S. Pat. No. 4,946,778, Kontermann and Dübel (2010), loc. cit. and Little (2009), loc. cit.) can be adapted to produce single chain constructs selectively and, preferably, specifically recognizing (an) elected target(s).

Bivalent (also called divalent) or bispecific single-chain variable fragments (bi-scFvs or di-scFvs) having the format (scFv)2 can be engineered by linking two scFv molecules (e.g. with linkers as described hereinbefore). The linking can be done by producing a single polypeptide chain with two VH regions and two VL regions, yielding tandem scFvs (see e.g. Kufer, P. et al., (2004) Trends in Biotechnology 22(5):238-244). Another possibility is the creation of scFv molecules with linker peptides that are too short for the two variable regions to fold together (e.g. about five amino acids), forcing the scFvs to dimerize. In this case, the VH and the VL of a binding domain (binding either to CD3epsilon or the further target antigen) are not directly connected via a peptide linker. Thus, the VH of the CD3 binding domain may e.g. be fused to the VL of the further target antigen binding domain via a peptide linker, and the VH of the further target antigen binding domain is fused to the VL of the CD3 binding domain via such peptide linker. This type is known as diabodies (see e.g. Hollinger, Philipp et al., (July 1993) Proceedings of the National Academy of Sciences of the United States of America 90 (14): 6444-8.).

In accordance with the invention, the polypeptide or polypeptide construct the invention comprises at least one further binding domain. In other words, the polypeptide or polypeptide construct comprises the CD3epsilon binding domain defined herein above and at least one further binding domain. Said further binding domain can be a further CD3 binding domain, preferably one as defined herein. As such, the polypeptide can comprise two of the same CD3 binding domains as defined herein. Alternatively or additionally (in a construct comprising at least three binding domains), the at least one further binding domain binds to a different target, such as a cell surface antigen.

Preferably, said at least one further binding domain binds to a cell surface antigen. The term “cell surface antigen” as used herein denotes a molecule, which is displayed on the surface of a cell. In most cases, this molecule will be located in or on the plasma membrane of the cell such that at least part of this molecule remains accessible from outside the cell in tertiary form. A non-limiting example of a cell surface molecule, which is located in the plasma membrane is a transmembrane protein comprising, in its tertiary conformation, regions of hydrophilicity and hydrophobicity. Here, at least one hydrophobic region allows the cell surface molecule to be embedded, or inserted in the hydrophobic plasma membrane of the cell while the hydrophilic regions extend on either side of the plasma membrane into the cytoplasm and extracellular space, respectively. It will be appreciated that an extracellular epitope refers to an epitope comprised by a portion of a protein that is disposed in the extracellular space, for example a portion of a cell surface molecule that extends into the extracellular space when that cell surface molecule is a native configuration disposed, embedded, or inserted in the plasma membrane. Non-limiting examples of cell surface molecules which are located on the plasma membrane are proteins which have been modified at a cysteine residue to bear a palmitoyl group, proteins modified at a C-terminal cysteine residue to bear a farnesyl group or proteins which have been modified at the C-terminus to bear a glycosyl phosphatidyl inositol (“GPI”) anchor.

Said cell surface antigen is preferably a tumor antigen. The term “tumor antigen” as used herein may be understood as those antigens that are presented on tumor cells. These antigens can be presented on the cell surface with an extracellular part, which is often combined with a transmembrane and cytoplasmic part of the molecule. These antigens can sometimes be presented only by tumor cells and never by the normal ones. Tumor antigens can be exclusively expressed on tumor cells or might represent a tumor specific mutation compared to normal cells. In this case, they are called tumor-specific antigens. More common are antigens that are presented by tumor cells and normal cells, and they are called tumor-associated antigens. These tumor-associated antigens can be overexpressed compared to normal cells or are accessible for antibody binding in tumor cells due to the less compact structure of the tumor tissue compared to normal tissue.

In a preferred embodiment, the tumor antigen is selected from the group consisting of BCMA (B-cell maturation antigen), CD123 (interleukin-3 receptor alpha chain (IL-3R)), CD19 (B-lymphocyte antigen CD19), CD20 (B-lymphocyte antigen CD20), CD22 (cluster of differentiation-22), CD33 (Siglec-3), CD70 (Cluster of Differentiation 70), CDH19 (Cadherin 19), CDH3 (Cadherin 3), CLL1 (C-type lectin domain family 12 member A), CS1 (CCND3 subset 1), CLDN6 (Claudin-6), CLDN18.2 (Claudin 18.2), DLL3 (Delta-like ligand 3), EGFRvII (Epidermal growth factor receptor vIII), FLT3 (fms like tyrosine kinase 3), MAGEB2 (Melanoma-associated antigen B2), MART1 (Melanoma Antigen Recognized By T-Cells 1), MSLN (Mesothelin), MUC17 (Mucin-17), PSMA (prostate-specific membrane antigen), and STEAP1 (Metalloreductase STEAP1). These tumor antigens are well known in the art due to their expression on tumor cells.

Preferred CDR sequences and VH/VL region sequences for BCMA binding domains as further binding domains of the polypeptide or polypeptide construct of the invention, and bispecific single chain molecule sequences of a polypeptide or polypeptide construct in accordance with the invention having a BCMA binding domain as further binding domain (with and without half-life extending domain) are defined in SEQ ID NOs: 2072 to 2095.

Preferred CDR sequences and VH/VL region sequences for CD123 binding domains as further binding domains of the polypeptide or polypeptide construct of the invention, and bispecific single chain molecule sequences of a polypeptide or polypeptide construct in accordance with the invention having a CD123 binding domain as further binding domain (with and without half-life extending domain) are defined in SEQ ID NOs: 2096 to 2110.

Preferred CDR sequences and VH/VL region sequences for CD19 binding domains as further binding domains of the polypeptide or polypeptide construct of the invention, and bispecific single chain molecule sequences of a polypeptide or polypeptide construct in accordance with the invention having a CD19 binding domain as further binding domain (with and without half-life extending domain) are defined in SEQ ID NOs: 2111 to 2125.

Preferred CDR sequences and VH/VL region sequences for CD20 binding domains as further binding domains of the polypeptide or polypeptide construct of the invention, and bispecific single chain molecule sequences of a polypeptide or polypeptide construct in accordance with the invention having a CD20 binding domain as further binding domain (with and without half-life extending domain) are defined in SEQ ID NOs: 2126 to 2140.

Preferred CDR sequences and VH/VL region sequences for CD22 binding domains as further binding domains of the polypeptide or polypeptide construct of the invention, and bispecific single chain molecule sequences of a polypeptide or polypeptide construct in accordance with the invention having a CD22 binding domain as further binding domain (with and without half-life extending domain) are defined in SEQ ID NOs: 2141 to 2154.

Preferred CDR sequences and VH/VL region sequences for CD33 binding domains as further binding domains of the polypeptide or polypeptide construct of the invention, and bispecific single chain molecule sequences of a polypeptide or polypeptide construct in accordance with the invention having a CD33 binding domain as further binding domain (with and without half-life extending domain) are defined in SEQ ID NOs: 2155 to 2178.

Preferred CDR sequences and VH/VL region sequences for CD70 binding domains as further binding domains of the polypeptide or polypeptide construct of the invention, and bispecific single chain molecule sequences of a polypeptide or polypeptide construct in accordance with the invention having a CD70 binding domain as further binding domain (with and without half-life extending domain) are defined in SEQ ID NOs: 2179 to 2192.

Preferred CDR sequences and VH/VL region sequences for CDH19 binding domains as further binding domains of the polypeptide or polypeptide construct of the invention, and bispecific single chain molecule sequences of a polypeptide or polypeptide construct in accordance with the invention having a CDH19 binding domain as further binding domain (with and without half-life extending domain) are defined in SEQ ID NOs: 2193 to 2212.

Preferred CDR sequences and VH/VL region sequences for CDH3 binding domains as further binding domains of the polypeptide or polypeptide construct of the invention, and bispecific single chain molecule sequences of a polypeptide or polypeptide construct in accordance with the invention having a CDH3 binding domain as further binding domain (with and without half-life extending domain) are defined in SEQ ID NOs: 2213 to 2256.

Preferred CDR sequences and VH/VL region sequences for CLL1 binding domains as further binding domains of the polypeptide or polypeptide construct of the invention, and bispecific single chain molecule sequences of a polypeptide or polypeptide construct in accordance with the invention having a CLL1 binding domain as further binding domain (with and without half-life extending domain) are defined in SEQ ID NOs: 2257 to 2271.

Preferred CDR sequences and VH/VL region sequences for CS1 binding domains as further binding domains of the polypeptide or polypeptide construct of the invention, and bispecific single chain molecule sequences of a polypeptide or polypeptide construct in accordance with the invention having a CS1 binding domain as further binding domain (with and without half-life extending domain) are defined in SEQ ID NOs: 2272 to 2285.

Preferred CDR sequences and VH/VL region sequences for CLDN6 binding domains as further binding domains of the polypeptide or polypeptide construct of the invention, and bispecific single chain molecule sequences of a polypeptide or polypeptide construct in accordance with the invention having a CLDN6 binding domain as further binding domain (with and without half-life extending domain) are defined in SEQ ID NOs: 2286 to 2299.

Preferred CDR sequences and VH/VL region sequences for CLDN18.2 binding domains as further binding domains of the polypeptide or polypeptide construct of the invention, and bispecific single chain molecule sequences of a polypeptide or polypeptide construct in accordance with the invention having a CLDN18.2 binding domain as further binding domain (with and without half-life extending domain) are defined in SEQ ID NOs: 2300 to 2314.

Preferred CDR sequences and VH/VL region sequences for DLL3 binding domains as further binding domains of the polypeptide or polypeptide construct of the invention, and bispecific single chain molecule sequences of a polypeptide or polypeptide construct in accordance with the invention having a DLL3 binding domain as further binding domain (with and without half-life extending domain) are defined in SEQ ID NOs: 2315 to 2328.

Preferred CDR sequences and VH/VL region sequences for EGFRvIII binding domains as further binding domains of the polypeptide or polypeptide construct of the invention, and bispecific single chain molecule sequences of a polypeptide or polypeptide construct in accordance with the invention having a EGFRvIII binding domain as further binding domain (with and without half-life extending domain) are defined in SEQ ID NOs: 2329 to 2342.

Preferred CDR sequences and VH/VL region sequences for FLT3 binding domains as further binding domains of the polypeptide or polypeptide construct of the invention, and bispecific single chain molecule sequences of a polypeptide or polypeptide construct in accordance with the invention having a FLT3 binding domain as further binding domain (with and without half-life extending domain) are defined in SEQ ID NOs: 2343 to 2357.

Preferred CDR sequences and VH/VL region sequences for MAGEB2 binding domains as further binding domains of the polypeptide or polypeptide construct of the invention, and bispecific single chain molecule sequences of a polypeptide or polypeptide construct in accordance with the invention having a MAGEB2 binding domain as further binding domain (with and without half-life extending domain) are defined in SEQ ID NOs: 2358 to 2378.

Preferred CDR sequences and VH/VL region sequences for MART1 binding domains as further binding domains of the polypeptide or polypeptide construct of the invention are defined in SEQ ID NOs: 2379 to 2387.

Preferred CDR sequences and VH/VL region sequences for MSLN binding domains as further binding domains of the polypeptide or polypeptide construct of the invention, and bispecific single chain molecule sequences of a polypeptide or polypeptide construct in accordance with the invention having a MSLN binding domain as further binding domain (with and without half-life extending domain) are defined in SEQ ID NOs: 2388 to 2431.

Preferred CDR sequences and VH/VL region sequences for MUC17 binding domains as further binding domains of the polypeptide or polypeptide construct of the invention, and bispecific single chain molecule sequences of a polypeptide or polypeptide construct in accordance with the invention having a MUC17 binding domain as further binding domain (with and without half-life extending domain) are defined in SEQ ID NOs: 2432 to 2445.

Preferred CDR sequences and VH/VL region sequences for PSMA binding domains as further binding domains of the polypeptide or polypeptide construct of the invention, and bispecific single chain molecule sequences of a polypeptide or polypeptide construct in accordance with the invention having a PSMA binding domain as further binding domain (with and without half-life extending domain) are defined in SEQ ID NOs: 2446 to 2475.

Preferred bispecific single chain molecule sequences of a polypeptide or polypeptide construct in accordance with the invention having CD20 and CD22 binding domains as further binding domains (with and without a further CD3 binding domain as defined herein) and a half-life extending domain are defined in SEQ ID NOs: 2505 to 2516.

Preferred bispecific single chain molecule sequences of a polypeptide or polypeptide construct in accordance with the invention having CS1 and BCMA binding domains as further binding domains and a half-life extending domain are defined in SEQ ID NOs: 2517 to 2522.

Preferred bispecific single chain molecule sequences of a polypeptide or polypeptide construct in accordance with the invention having MSLN and CDH3 binding domains as further binding domains and a half-life extending domain are defined in SEQ ID NOs: 2529 to 2536.

As laid out herein above, the polypeptide construct of the invention comprises a binding domain which binds to CD3 on the surface of a T cell. “CD3” (cluster of differentiation 3) is a T cell co-receptor composed of four chains. In mammals, the CD3 protein complex contains a CD3γ (gamma) chain, a CD3δ (delta) chain, and two CD3ε (epsilon) chains. These four chains associate with the T cell receptor (TCR) and the so-called ((zeta) chain to for the “T cell receptor complex” and to generate an activation signal in T lymphocytes. The CD3γ (gamma), CD3δ (delta), and CD3ε (epsilon) chains are highly related cell-surface proteins of the immunoglobulin superfamily and each contain a single extracellular immunoglobulin domain. The intracellular tails of the CD3 molecules contain a single conserved motif known as an immunoreceptor tyrosine-based activation motif (ITAM), which is essential for the signaling capacity of the TCR. The CD3 epsilon molecule is a polypeptide which in humans is encoded by the CD3 epsilon gene which resides on chromosome 11. In the context of the present invention, CD3 is understood as a protein complex and T cell co-receptor that is involved in activating both the cytotoxic T cell (CD8+ naive T cells) and T helper cells (CD4+ naive T cells). It is typically composed of four distinct chains. Especially in mammals, the complex contains a CD3γ chain, a CD3δ chain, and two CD3ε chains. These chains associate with the T-cell receptor (TCR) and the ζ-chain (zeta-chain) to generate an activation signal in T lymphocytes. The TCR, ζ-chain, and CD3 molecules together constitute the TCR complex.

The redirected lysis of target cells via the recruitment of T cells by a construct which binds to CD3 on the T cell and to a target protein on the target cell generally involves cytolytic synapse formation and delivery of perforin and granzymes. The engaged T cells are capable of serial target cell lysis and are not affected by immune escape mechanisms interfering with peptide antigen processing and presentation, or clonal T cell differentiation; see e.g. WO 2007/042261.

Cytotoxicity mediated by given tumor antigen×CD3 constructs can be measured in various ways. The “half maximal effective concentration” (EC50) is commonly used as a measure of potency of a biologically active molecule such as a construct of the present invention. It may be expressed in molar units. In the present case of measuring cytotoxicity, the EC50 value refers to the concentration of a construct inducing a cytotoxic response (lysis of target cells) halfway between the baseline and the maximum. Effector cells in a cytotoxicity assay can e.g. be stimulated enriched (human) CD8 positive T cells or unstimulated (human) peripheral blood mononuclear cells (PBMC). An EC50 value may typically be expected to be lower when stimulated/enriched CD8+ T cells are used as effector cells, compared with unstimulated PBMC. If the target cells are of macaque origin or express or are transfected with a given macaque tumor antigen, the effector cells should also be of macaque origin, such as a macaque T cell line, e.g. 4119LnPx. The target cells should express said tumor antigen on the cell surface. Target cells can be a cell line (such as CHO) which is stably or transiently transfected with said tumor antigen. Alternatively, the target cells can be a tumor antigen positive natural expresser cell line, such as the human cancer lines. Usually EC50 values are expected to be lower when using target cells that express higher levels of said tumor antigen on the cell surface compared with target cells having a lower target expression rate.

The effector to target cell (E:T) ratio in a cytotoxicity assay is usually about 10:1, but can also vary. Cytotoxic activity of tumor antigen×CD3 constructs can be measured in a 51-chromium release assay (e.g. with an incubation time of about 18 hours) or in a in a FACS-based cytotoxicity assay (e.g. with an incubation time of about 48 hours). Modifications of the incubation time (cytotoxic reaction) are also envisaged. Other methods of measuring cytotoxicity are well-known and comprise MTT or MTS assays, ATP-based assays including bioluminescent assays, the sulforhodamine B (SRB) assay, WST assay, clonogenic assay and the ECIS technology.

According to one embodiment, the cytotoxic activity mediated by tumor antigen×CD3 constructs of the present invention is measured in a cell-based cytotoxicity assay. It may also be measured in a 51-chromium release assay. It is envisaged that the EC50 value of the constructs of the invention is ≤300 pM, ≤280 pM, ≤260 pM, ≤250 pM, ≤240 pM, ≤220 pM, ≤200 pM, ≤180 pM, ≤160 pM, ≤150 pM, ≤140 pM, ≤120 pM, ≤100 pM, ≤90 pM, ≤80 pM, ≤70 pM, ≤60 pM, ≤50 pM, ≤40 pM, ≤30 pM, ≤20 pM, ≤15 pM, ≤10 pM, or ≤5 pM.

The above given EC50 values can be measured in different assays and under different conditions. For example, when human PBMCs are used as effector cells and tumor antigen transfected cells such as CHO cells are used as target cells, it is envisaged that the EC50 value of the tumor antigen×CD3 construct is ≤500 pM, ≤400 pM, ≤300 pM, ≤280 pM, ≤260 pM, ≤250 pM, ≤240 pM, ≤220 pM, ≤200 pM, ≤180 pM, ≤160 pM, ≤150 pM, ≤140 pM, ≤120 pM, ≤100 pM, ≤90 pM, ≤80 pM, ≤70 pM, ≤60 pM, ≤50 pM, ≤40 pM, ≤30 pM, ≤20 pM, ≤15 pM, ≤10 pM, or 55 pM. When human PBMCs are used as effector cells and when the target cells are a CLDN6 positive cell line such as, it is envisaged that the EC50 value of the CLDN6×CD3 construct is ≤300 pM, ≤280 pM, ≤260 pM, ≤250 pM, ≤240 pM, ≤220 pM, ≤200 pM, ≤180 pM, ≤160 pM, ≤150 pM, ≤140 pM, ≤120 pM, ≤100 pM, ≤90 pM, ≤80 pM, ≤70 pM, ≤60 pM, ≤50 pM, ≤40 pM, ≤30 pM, ≤20 pM, ≤15 pM, ≤10 pM, or ≤5 pM.

According to one embodiment, the tumor antigen×CD3 polypeptides/polypeptide constructs of the present invention do not induce/mediate lysis or do not essentially induce/mediate lysis of cells that do not express said given tumor antigen on their surface (tumor antigen negative cells), such as CHO cells. The term “do not induce lysis”, “do not essentially induce lysis”, “do not mediate lysis” or “do not essentially mediate lysis” means that a construct of the present invention does not induce or mediate lysis of more than 30%, preferably not more than 20%, more preferably not more than 10%, particularly preferably not more than 9%, 8%, 7%, 6% or 5% of tumor antigen negative cells, whereby lysis of tumor antigen expressing target cells (such as cells transformed or transfected with said tumor antigen or a natural expresser cell line such as the human cancer lines) is set to be 100%. This usually applies for concentrations of the construct of up to 500 nM. Cell lysis measurement is a routine technique. Moreover, the present specification teaches specific instructions how to measure cell lysis.

The difference in cytotoxic activity between the monomeric and the dimeric isoform of individual tumor antigen×CD3 polypeptides/polypeptide constructs is referred to as “potency gap”. This potency gap can e.g. be calculated as ratio between EC50 values of the molecule's monomeric and dimeric form. In one method to determine this gap, an 18 hour 51-chromium release assay or a 48h FACS-based cytotoxicity assay is carried out as described hereinbelow with purified construct monomer and dimer. Effector cells are stimulated enriched human CD8+ T cells or unstimulated human PBMC. Target cells are hu tumor antigen transfected CHO cells. Effector to target cell (E:T) ratio is 10:1. Potency gaps of the tumor antigen×CD3 constructs of the present invention are preferably ≤5, more preferably s 4, even more preferably s 3, even more preferably s 2 and most preferably s 1.

The binding domain(s) of the polypeptide construct of the invention is/are preferably cross-species specific for members of the mammalian order of primates, such as macaques. According to one embodiment, the further binding domain(s), in addition to binding to a human tumor antigen, will also bind to said tumor antigen of primates including (but not limited to) new world primates (such as Callithrix jacchus, Saguinus Oedipus or Saimiri sciureus), old world primates (such as baboons and macaques), gibbons, orangutans and non-human hominidae. It is envisaged that the domain which binds to human CD3 on the surface of a T cell of the invention also binds at least to macaque CD3. A preferred macaque is Macaca fascicularis. Macaca mulatta (Rhesus) is also envisaged. The polypeptide or polypeptide construct of the invention comprises a domain which binds to human CD3epsilon on the surface of a T cell and at least macaque CD3.

In one embodiment, the affinity gap of the constructs according to the invention for binding macaque CD3 versus human CD3 [KD ma CD3: KD hu CD3] (as determined e.g. by BiaCore or by Scatchard analysis) is between 0.01 and 100, preferably between 0.1 and 10, more preferably between 0.2 and 5, more preferably between 0.3 and 4, even more preferably between 0.5 and 3 or between 0.5 and 2.5, and most preferably between 0.5 and 1.

As detailed herein above, said binding domain of the polypeptide or polypeptide construct of the invention binds to human CD3 epsilon (or human CD3 epsilon on the surface of a T cell) and, preferably, to Callithrix jacchus or Saimiri sciureus CD3 epsilon. More specifically, said domain binds to an extracellular epitope of human CD3 epsilon. It is also envisaged that said domain binds to an extracellular epitope of the human and the Macaca CD3 epsilon chain. Said extracellular epitope of CD3 epsilon is comprised within amino acid residues 1-27 of the human CD3 epsilon extracellular domain (see SEQ ID NO: 2552; amino acid residues 1-27 in SEQ ID NO: 2553). Even more particularly, the epitope comprises at least the amino acid sequence Gln-Asp-Gly-Asn-Glu. Callithrix jacchus is a new world primate belonging to the family of Callitrichidae, while Saimiri sciureus is a new world primate belonging to the family of Cebidae.

In a preferred embodiment, the polypeptide or polypeptide construct of the invention is a single chain polypeptide that is at least bispecific. It is preferred for this embodiment that the CD3 binding domain according to the invention is present as a scFv in the polypeptide or polypeptide construct.

It is also envisaged that the polypeptide construct of the invention has, in addition to its function to bind to CD3 and, in certain embodiments to bind to at least a further binding domain, a further function. In this format, the construct may be a trifunctional or multifunctional construct by targeting target cells through, preferably, tumor antigen binding, mediating cytotoxic T cell activity through CD3 binding and providing a further function such as means or domains to enhance or extend serum half-life, a fully functional or modified Fc constant domain mediating cytotoxicity through recruitment of effector cells, a label (fluorescent etc.), a therapeutic agent such as a toxin or radionuclide, etc.

Examples for means or domains to extend serum half-life of the polypeptides/polypeptide constructs of the invention include peptides, proteins or domains of proteins, which are fused or otherwise attached to the polypeptides/polypeptide constructs. The group of peptides, proteins or protein domains includes peptides binding to other proteins with preferred pharmacokinetic profile in the human body such as serum albumin (see WO 2009/127691). An alternative concept of such half-life extending peptides includes peptides binding to the neonatal Fc receptor (FcRn, see WO 2007/098420), which can also be used in the constructs of the present invention. The concept of attaching larger domains of proteins or complete proteins includes the fusion of human serum albumin, variants or mutants of human serum albumin (see WO 2011/051489, WO 2012/059486, WO 2012/150319, WO 2013/135896, WO 2014/072481, WO 2013/075066) or domains thereof, as well as the fusion of an immunoglobulin constant region (Fc domain) and variants thereof. Such variants of Fc domains are called Fc-based domains and may e.g. be optimized/modified to allow the desired pairing of dimers or multimers, to abolish Fc receptor binding (e.g. to avoid ADCC or CDC) or for other reasons. A further concept known in the art to extend the half-life of substances or molecules in the human body is the pegylation of those molecules (such as the constructs of the present invention).

In one embodiment, the polypeptides/polypeptide constructs according to the invention are linked (e.g. via peptide bond) with a fusion partner (such as a protein, polypeptide or peptide), e.g. for extending the construct's serum half-life. These fusion partners can be selected from human serum albumin (“HSA” or “HALB”) as wells as sequence variants thereof, peptides binding to HSA, peptides binding to FcRn (“FcRn BP”), or constructs comprising an (antibody derived) Fc region. Exemplary sequences of these fusion partners are depicted in SEQ ID NOs: 16, 18 and 19. In general, the fusion partners may be linked to the N-terminus or to the C-terminus of the constructs according to the invention, either directly (e.g. via peptide bond) or through a peptide linker such as (GGGGS)n or (GGGGQ)n (wherein “n” is an integer of 2 or greater, e.g. 2 or 3 or 4). Suitable peptide linkers are discussed above and are shown in SEQ ID NOs: 2 and 2551.

It is envisaged that a polypeptide construct according to the present invention comprises a single chain polypeptide that is at least bispecific, wherein said polypeptide comprises or consists of in the following order from N-terminus to C-terminus:
a) VL (comprising part of a cell surface antigen binding domain/paratope)-(G4S)3 or (G4Q)3-VH (comprising part of a cell surface antigen binding domain/paratope)-Peptide linker (SG4S) or (SG4Q)-VH (comprising part of the CD3epsilon binding domain/paratope)-(G4S)3 or (G4Q)3-VL (comprising part of the CD3epsilon binding domain/paratope);
b) VH (comprising part of a cell surface antigen binding domain/paratope)-(G4S)3 or (G4Q)3-VL (comprising part of a cell surface antigen binding domain/paratope)-Peptide linker (SG4S) or (SG4Q)-VH (comprising part of the CD3epsilon binding domain/paratope)-(G4S)3 or (G4Q)3-VL (comprising part of the CD3epsilon binding domain/paratope);
c) VL (comprising part of a cell surface antigen binding domain/paratope)-(G4S)3 or (G4Q)3-VH (comprising part of a cell surface antigen binding domain/paratope)-Peptide linker (SG4S) or (SG4Q)-VH (comprising part of the CD3epsilon binding domain/paratope)-(G4S)3 or (G4Q)3-VL (comprising part of the CD3epsilon binding domain/paratope)-Peptide linker (G4)-Fc monomer (part of the HLE domain)-(G4S)6 or (G4Q)6 or -Fc monomer (part of the HLE domain);
d) VH (comprising part of a cell surface antigen binding domain/paratope)-(G4S)3 or (G4Q)3-VL (comprising part of a cell surface antigen binding domain/paratope)-Peptide linker (SG4S) or (SG4Q)-VH (comprising part of the CD3epsilon binding domain/paratope)-(G4S)3 or (G4Q)3-VL (comprising part of the CD3epsilon binding domain/paratope)-Peptide linker (G4)-Fc monomer (part of the HLE domain)-(G4S)6 or (G4Q)6 or -Fc monomer (part of the HLE domain);
e) VH (comprising part of a first cell surface antigen binding domain/paratope)-(G4S)3 or (G4Q)3-VL (comprising part of a first cell surface antigen binding domain/paratope)-Peptide linker (SG4S) or (SG4Q)-VL (comprising part of a second cell surface antigen binding domain/paratope)-(G4S)3 or (G4Q)3-VH (comprising part of a second cell surface antigen binding domain/paratope)-Peptide linker (SG4S) or (SG4Q)-VH (comprising part of the CD3epsilon binding domain/paratope)-(G4S)3 or (G4Q)3-VL (comprising part of the CD3epsilon binding domain/paratope)-Peptide linker (G4)-Fc monomer (part of the HLE domain)-(G4S)6 or (G4Q)6 or -Fc monomer (part of the HLE domain);
f) VH (comprising part of a first cell surface antigen binding domain/paratope)-(G4S)3 or (G4Q)3-VL (comprising part of a first cell surface antigen binding domain/paratope)-Peptide linker (SG4S) or (SG4Q)-VH (comprising part of a second cell surface antigen binding domain/paratope)-(G4S)3 or (G4Q)3-VL (comprising part of a second cell surface antigen binding domain/paratope)-Peptide linker (SG4S) or (SG4Q)-VH (comprising part of the CD3epsilon binding domain/paratope)-(G4S)3 or (G4Q)3-VL (comprising part of the CD3epsilon binding domain/paratope)-Peptide linker (G4)-Fc monomer (part of the HLE domain)-(G4S)6 or (G4Q)6 or -Fc monomer (part of the HLE domain);
g) VL (comprising part of a first cell surface antigen binding domain/paratope)-(G4S)3 or (G4Q)3-VH (comprising part of a first cell surface antigen binding domain/paratope)-Peptide linker (SG4S) or (SG4Q)-VL (comprising part of a second cell surface antigen binding domain/paratope)-(G4S)3 or (G4Q)3-VH (comprising part of a second cell surface antigen binding domain/paratope)-Peptide linker (SG4S) or (SG4Q)-VH (comprising part of the CD3epsilon binding domain/paratope)-(G4S)3 or (G4Q)3-VL (comprising part of the CD3epsilon binding domain/paratope)-Peptide linker (G4)-Fc monomer (part of the HLE domain)-(G4S)6 or (G4Q)6 or -Fc monomer (part of the HLE domain)
h) VL (comprising part of a first cell surface antigen binding domain/paratope)-(G4S)3 or (G4Q)3-VH (comprising part of a first cell surface antigen binding domain/paratope)-Peptide linker (SG4S) or (SG4Q)-VH (comprising part of a second cell surface antigen binding domain/paratope)-(G4S)3 or (G4Q)3-VL (comprising part of a second cell surface antigen binding domain/paratope)-Peptide linker (SG4S) or (SG4Q)-VH (comprising part of the CD3epsilon binding domain/paratope)-(G4S)3 or (G4Q)3-VL (comprising part of the CD3epsilon binding domain/paratope)-Peptide linker (G4)-Fc monomer (part of the HLE domain)-(G4S)6 or (G4Q)6 or -Fc monomer (part of the HLE domain); or
i) Binding domain 1 ((VL (comprising part of a first cell surface antigen binding domain/paratope)-(G4S)3 or (G4Q)3-VH (comprising part of a first cell surface antigen binding domain/paratope)) or (VH (comprising part of a first cell surface antigen binding domain/paratope)-(G4S)3 or (G4Q)3-VL (comprising part of a first cell surface antigen binding domain/paratope)))-Peptide linker (G4S) or (G4Q)-CD3 binding domain 1 (VH (comprising part of a first CD3epsilon binding domain/paratope)-(G4S)3 or (G4Q)3-VL (comprising part of a first CD3epsilon binding domain/paratope))-Peptide linker (G4)-Fc monomer (part of the HLE domain)-(G4S)6 or (G4Q)6-Fc monomer (part of the HLE domain)-Peptide linker (G4)-Binding domain 2 ((VL (comprising part of a second cell surface antigen binding domain/paratope)-(G4S)3 or (G4Q)3-VH (comprising part of a first cell surface antigen binding domain/paratope)) or (VH (comprising part of a second cell surface antigen binding domain/paratope)-(G4S)3 or (G4Q)3-VL (comprising part of a second cell surface antigen binding domain/paratope)))—Peptide linker (G4S) or (G4Q)-CD3 binding domain 2 (VH (comprising part of a second CD3epsilon binding domain/paratope)-(G4S)3 or (G4Q)3-VL (comprising part of a second CD3epsilon binding domain/paratope)).

As is evident from the above, the VH and VL region sequence orientation of the binding domain(s) of the cell surface antigen can be VH-VL or VL-VH. Preferably, the cell surface antigen is a tumor antigen as detailed herein above. The HLE domain sequences made up of the Fc monomers and connecting linkers as detailed are preferably selected from the sequences as defined in SEQ ID NOs: 18 and 19. The two CD3 binding domains of the polypeptide construct of item i) are preferably the same CD3 binding domains, such as, preferably, the CD3 binding domain with VH and VL region sequences of SEQ ID NOs: 2028 and 2029. While peptide linkers (SG4S) or (SG4Q) are preferred at the indicated positions, they can also be replaced by (G4S) or (G4Q) linkers.

Covalent modifications of the polypeptides/polypeptide constructs are also included within the scope of this invention, and are generally, but not always, done post-translationally. For example, several types of covalent modifications of the construct are introduced into the molecule by reacting specific amino acid residues of the construct with an organic derivatizing agent that can react with selected side chains or with the N- or C-terminal residues. Derivatization with bifunctional agents is useful for crosslinking the constructs of the present invention to a water-insoluble support matrix or surface for use in a variety of methods. Glutaminyl and asparaginyl residues are frequently deamidated to the corresponding glutamyl and aspartyl residues, respectively. Alternatively, these residues are deamidated under mildly acidic conditions. Either form of these residues falls within the scope of this invention. Other modifications include hydroxylation of proline and lysine, phosphorylation of hydroxyl groups of seryl or threonyl residues, methylation of the α-amino groups of lysine, arginine, and histidine side chains (T. E. Creighton, Proteins: Structure and Molecular Properties, W. H. Freeman & Co., San Francisco, 1983, pp. 79-86), acetylation of the N-terminal amine, and amidation of any C-terminal carboxyl group.

Another type of covalent modification of the constructs included within the scope of this invention comprises altering the glycosylation pattern of the protein. As is known in the art, glycosylation patterns can depend on both the sequence of the protein (e.g., the presence or absence of specific glycosylation amino acid residues, discussed below), or the host cell or organism in which the protein is produced. Specific expression systems are discussed below. Glycosylation of polypeptides is typically either N-linked or O-linked. N-linked refers to the attachment of the carbohydrate moiety to the side chain of an asparagine residue. The tri-peptide sequences asparagine-X-serine and asparagine-X-threonine, where X is any amino acid except proline, are the recognition sequences for enzymatic attachment of the carbohydrate moiety to the asparagine side chain. Thus, the presence of either of these tri-peptide sequences in a polypeptide creates a potential glycosylation site. O-linked glycosylation refers to the attachment of one of the sugars N-acetylgalactosamine, galactose, or xylose, to a hydroxyamino acid, most commonly serine or threonine, although 5-hydroxyproline or 5-hydroxylysine may also be used.

Addition of glycosylation sites to the construct is conveniently accomplished by altering the amino acid sequence such that it contains one or more of the above-described tri-peptide sequences (for N-linked glycosylation sites). The alteration may also be made by the addition of, or substitution by, one or more serine or threonine residues to the starting sequence (for O-linked glycosylation sites). For ease, the amino acid sequence of a construct may be altered through changes at the DNA level, particularly by mutating the DNA encoding the polypeptide at preselected bases such that codons are generated that will translate into the desired amino acids.

Another means of increasing the number of carbohydrate moieties on the construct is by chemical or enzymatic coupling of glycosides to the protein. These procedures are advantageous in that they do not require production of the protein in a host cell that has glycosylation capabilities for N- and O-linked glycosylation. Depending on the coupling mode used, the sugar(s) may be attached to (a) arginine and histidine, (b) free carboxyl groups, (c) free sulfhydryl groups such as those of cysteine, (d) free hydroxyl groups such as those of serine, threonine, or hydroxyproline, (e) aromatic residues such as those of phenylalanine, tyrosine, or tryptophan, or (f) the amide group of glutamine. These methods are described in WO 87/05330, and in Aplin and Wriston, 1981, CRC Crit. Rev. Biochem., pp. 259-306.

Removal of carbohydrate moieties present on the starting construct may be accomplished chemically or enzymatically. Chemical deglycosylation requires exposure of the protein to the compound trifluoromethanesulfonic acid, or an equivalent compound. This treatment results in the cleavage of most or all sugars except the linking sugar (N-acetylglucosamine or N-acetylgalactosamine), while leaving the polypeptide intact. Chemical deglycosylation is described by Hakimuddin et al., 1987, Arch. Biochem. Biophys. 259:52 and by Edge et al., 1981, Anal. Biochem. 118:131. Enzymatic cleavage of carbohydrate moieties on polypeptides can be achieved using a variety of endo- and exo-glycosidases as described by Thotakura et al., 1987, Meth. Enzymol. 138:350. Glycosylation at potential glycosylation sites may be prevented using the compound tunicamycin as described by Duskin et al., 1982, J. Biol. Chem. 257:3105. Tunicamycin blocks the formation of protein-N-glycoside linkages.

Other modifications of the construct are also contemplated herein. For example, another type of covalent modification of the construct comprises linking the construct to various non-proteinaceous polymers, including polyols, in the manner set forth in U.S. Pat. Nos. 4,640,835; 4,496,689; 4,301,144; 4,670,417; 4,791,192 or 4,179,337. In addition, as is known in the art, amino acid substitutions may be made in various positions within the construct, e.g. to facilitate the addition of polymers such as polyethylene glycol (PEG).

In some embodiments, the covalent modification of the constructs of the invention comprises the addition of one or more labels. The labelling group may be coupled to the construct via spacer arms of various lengths to reduce potential steric hindrance. Various methods for labelling proteins are known in the art and can be used in performing the present invention. The term “label” or “labelling group” refers to any detectable label. In general, labels fall into a variety of classes, depending on the assay in which they are to be detected—the following examples include, but are not limited to:
a) isotopic labels, which may be radioactive or heavy isotopes, such as radioisotopes or radionuclides (e.g., ³H, ¹⁴C ¹⁵N, S, ⁸⁹Zr, ⁹⁰, ⁹⁹Tc, ¹¹¹In, ¹²⁵I, ¹³¹I)
b) magnetic labels (e.g., magnetic particles)
c) redox active moieties
d) optical dyes (including, but not limited to, chromophores, phosphors and fluorophores) such as fluorescent groups (e.g., FITC, rhodamine, lanthanide phosphors), chemiluminescent groups, and fluorescent labels or fluorophores which can be either “small molecule” fluores or proteinaceous fluores
e) enzymatic groups (e.g. horseradish peroxidase, β-galactosidase, luciferase, alkaline phosphatase)
f) biotinylated groups
g) predetermined polypeptide epitopes recognized by a secondary reporter (e.g., leucine zipper pair sequences, binding sites for secondary antibodies, metal binding domains, epitope tags, etc.).

By “fluorescent label” is meant any molecule that may be detected via its inherent fluorescent properties. Suitable fluorescent labels include, but are not limited to, fluorescein, rhodamine, tetramethylrhodamine, eosin, erythrosin, coumarin, methyl-coumarins, pyrene, Malacite green, stilbene, Lucifer Yellow, Cascade BlueJ, Texas Red, IAEDANS, EDANS, BODIPY FL, LC Red 640, Cy 5, Cy 5.5, LC Red 705, Oregon green, the Alexa-Fluor dyes (Alexa Fluor 350, Alexa Fluor 430, Alexa Fluor 488, Alexa Fluor 546, Alexa Fluor 568, Alexa Fluor 594, Alexa Fluor 633, Alexa Fluor 660, Alexa Fluor 680), Cascade Blue, Cascade Yellow and R-phycoerythrin (PE) (Molecular Probes, Eugene, OR), FITC, Rhodamine, and Texas Red (Pierce, Rockford, IL), Cy5, Cy5.5, Cy7 (Amersham Life Science, Pittsburgh, PA). Suitable optical dyes, including fluorophores, are described in Molecular Probes Handbook by Richard P. Haugland.

Suitable proteinaceous fluorescent labels also include, but are not limited to, green fluorescent protein, including a Renilla, Ptilosarcus, or Aequorea species of GFP (Chalfie et al., 1994, Science 263:802-805), EGFP (Clontech Laboratories, Inc., Genbank® Accession Number U55762), blue fluorescent protein (BFP, Quantum Biotechnologies, Inc. 1801 de Maisonneuve Blvd. West, 8th Floor, Montreal, Quebec, Canada H3H 1J9; Stauber, 1998, Biotechniques 24:462-471; Heim et al., 1996, Curr. Biol. 6:178-182), enhanced yellow fluorescent protein (EYFP, Clontech Laboratories, Inc.), luciferase (Ichiki et al., 1993, J. Immunol. 150:5408-5417), p galactosidase (Nolan et al., 1988, Proc. Natl. Acad. Sci. U.S.A. 85:2603-2607) and Renilla (WO92/15673, WO95/07463, WO98/14605, WO98/26277, WO99/49019, U.S. Pat. Nos. 5,292,658; 5,418,155; 5,683,888; 5,741,668; 5,777,079; 5,804,387; 5,874,304; 5,876,995; 5,925,558).

Leucine zipper domains are peptides that promote oligomerization of the proteins in which they are found. Leucine zippers were originally identified in several DNA-binding proteins (Landschulz et al., 1988, Science 240:1759), and have since been found in a variety of different proteins. Among the known leucine zippers are naturally occurring peptides and derivatives thereof that dimerize or trimerize. Examples of leucine zipper domains suitable for producing soluble oligomeric proteins are described in PCT application WO 94/10308, and the leucine zipper derived from lung surfactant protein D (SPD) described in Hoppe et al., 1994, FEBS Letters 344:191. The use of a modified leucine zipper that allows for stable trimerization of a heterologous protein fused thereto is described in Fanslow et al., 1994, Semin. Immunol. 6:267-78.

The polypeptide construct of the invention may also comprise additional domains, which are e.g. helpful in the isolation of the molecule or relate to an adapted pharmacokinetic profile of the molecule. Domains helpful for the isolation of a construct may be selected from peptide motives or secondarily introduced moieties, which can be captured in an isolation method, e.g. an isolation column. Non-limiting embodiments of such additional domains comprise peptide motives known as Myc-tag, HAT-tag, HA-tag, TAP-tag, GST-tag, chitin binding domain (CBD-tag), maltose binding protein (MBP-tag), Flag-tag, Strep-tag and variants thereof (e.g. StrepII-tag) and His-tag. All herein disclosed constructs characterized by the identified CDRs may comprise a His-tag domain, which is generally known as a repeat of consecutive His residues in the amino acid sequence of a molecule, e.g. of five His residues, or of six His residues (hexa-histidine). The His-tag may be located e.g. at the N- or C-terminus of the construct. In one embodiment, a hexa-histidine tag (HHHHHH) is linked via peptide bond to the C-terminus of the construct according to the invention. A histidine tag is preferred, especially a 6×His tag.

The invention also relates to a polynucleotide encoding a polypeptide or polypeptide construct of the invention. Nucleic acid molecules are biopolymers composed of nucleotides. A polynucleotide is a biopolymer composed of 13 or more nucleotide monomers covalently bonded in a chain. DNA (such as cDNA) and RNA (such as mRNA) are examples of polynucleotides/nucleic acid molecules with distinct biological function. Nucleotides are organic molecules that serve as the monomers or subunits of nucleic acid molecules like DNA or RNA. The nucleic acid molecule or polynucleotide of the present invention can be double stranded or single stranded, linear or circular. It is envisaged that the nucleic acid molecule or polynucleotide is comprised in a vector. It is furthermore envisaged that such vector is comprised in a host cell. Said host cell is, e.g. after transformation or transfection with the vector or the polynucleotide/nucleic acid molecule of the invention, capable of expressing the construct. For this purpose, the polynucleotide or nucleic acid molecule is operatively linked with control sequences.

The genetic code is the set of rules by which information encoded within genetic material (nucleic acids) is translated into proteins. Biological decoding in living cells is accomplished by the ribosome which links amino acids in an order specified by mRNA, using tRNA molecules to carry amino acids and to read the mRNA three nucleotides at a time. The code defines how sequences of these nucleotide triplets, called codons, specify which amino acid will be added next during protein synthesis. With some exceptions, a three-nucleotide codon in a nucleic acid sequence specifies a single amino acid. Because the vast majority of genes are encoded with exactly the same code, this particular code is often referred to as the canonical or standard genetic code.

Degeneracy of codons is the redundancy of the genetic code, exhibited as the multiplicity of three-base pair codon combinations that specify an amino acid. Degeneracy results because there are more codons than encodable amino acids. The codons encoding one amino acid may differ in any of their three positions; however, often this difference is in the second or third position. For instance, codons GAA and GAG both specify glutamic acid and exhibit redundancy; but, neither specifies any other amino acid nor thus demonstrate ambiguity. The genetic codes of different organisms can be biased towards using one of the several codons that encode the same amino acid over the others—that is, a greater frequency of one will be found than expected by chance. For example, leucine is specified by six distinct codons, some of which are rarely used. Codon usage tables detailing genomic codon usage frequencies for most organisms are available. Recombinant gene technologies commonly take advantage of this effect by implementing a technique termed codon optimization, in which those codons are used to design a polynucleotide which are preferred by the respective host cell (such as a cell of human hamster origin, an Escherichia coli cell, or a Saccharomyces cerevisiae cell), e.g. to increase protein expression. It is hence envisaged that the polynucleotides/nucleic acid molecules of the present disclosure are codon optimized. Nevertheless, the polynucleotide/nucleic acid molecule encoding a construct of the invention may be designed using any codon that encodes the desired amino acid.

According to one embodiment, the polynucleotide/nucleic acid molecule of the present invention encoding the polypeptide construct of the invention is in the form of one single molecule or in the form of two or more separate molecules. If the construct of the present invention is a single chain construct, the polynucleotide/nucleic acid molecule encoding such construct will most likely also be in the form of one single molecule. However, it is also envisaged that different components of the polypeptide construct (such as the different domains, e.g. the paratope (antigen-binding (epitope-binding) structure)-comprising domain which binds to a cell surface antigen, the paratope (antigen-binding (epitope-binding) structure)-comprising domain which binds to CD3, and/or further domains such as antibody constant domains) are located on separate polypeptide chains, in which case the polynucleotide/nucleic acid molecule is most likely in the form of two or more separate molecules.

The same applies for the vector comprising a polynucleotide/nucleic acid molecule of the present invention. If the construct of the present invention is a single chain construct, one vector may comprise the polynucleotide which encodes the construct in one single location (as one single open reading frame, ORF). One vector may also comprise two or more polynucleotides/nucleic acid molecules at separate locations (with individual ORFs), each one of them encoding a different component of the construct of the invention. It is envisaged that the vector comprising the polynucleotide/nucleic acid molecule of the present invention is in the form of one single vector or two or more separate vectors. In one embodiment, and for the purpose of expressing the construct in a host cell, the host cell of the invention should comprise the polynucleotide/nucleic acid molecule encoding the construct or the vector comprising such polynucleotide/nucleic acid molecule in their entirety, meaning that all components of the construct—whether encoded as one single molecule or in separate molecules/locations—will assemble after translation and form together the biologically active construct of the invention.

The invention further relates to a vector comprising a polynucleotide/nucleic acid molecule of the invention. A vector is a nucleic acid molecule used as a vehicle to transfer (foreign) genetic material into a cell, usually to ensure the replication and/or expression of the genetic material. The term “vector” encompasses—but is not restricted to—plasmids, viruses, cosmids, and artificial chromosomes. Some vectors are designed specifically for cloning (cloning vectors), others for protein expression (expression vectors). So-called transcription vectors are mainly used to amplify their insert. The manipulation of DNA is normally conducted on E. coli vectors, which contain elements necessary for their maintenance in E. coli. However, vectors may also have elements that allow them to be maintained in another organism such as yeast, plant or mammalian cells, and these vectors are called shuttle vectors. Insertion of a vector into the target or host cell is usually called transformation for bacterial cells and transfection for eukaryotic cells, while insertion of a viral vector is often called transduction.

In general, engineered vectors comprise an origin of replication, a multicloning site and a selectable marker. The vector itself is generally a nucleotide sequence, commonly a DNA sequence, that comprises an insert (transgene) and a larger sequence that serves as the “backbone” of the vector. While the genetic code determines the polypeptide sequence for a given coding region, other genomic regions can influence when and where these polypeptides are produced. Modern vectors may therefore encompass additional features besides the transgene insert and a backbone: promoter, genetic marker, antibiotic resistance, reporter gene, targeting sequence, protein purification tag. Vectors called expression vectors (expression constructs) specifically are for the expression of the transgene in the target cell, and generally have control sequences.

The term “control sequences” refers to DNA sequences necessary for the expression of an operably linked coding sequence in a specific host organism. The control sequences that are suitable for prokaryotes, for example, include a promoter, optionally an operator sequence, and a ribosome binding site. Eukaryotic cells are known to utilize promoters, polyadenylation signals, a Kozak sequence and enhancers.

A nucleic acid is “operably linked” when it is placed into a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned to facilitate translation. Generally, “operably linked” means that the nucleotide sequences being linked are contiguous, and, in the case of a secretory leader, contiguous and in reading phase. However, enhancers do not have to be contiguous. Linking is accomplished by ligation at convenient restriction sites. If such sites do not exist, the synthetic oligonucleotide adaptors or linkers are used in accordance with conventional practice.

“Transfection” is the process of deliberately introducing nucleic acid molecules or polynucleotides (including vectors) into target cells. The term is mostly used for non-viral methods in eukaryotic cells. Transduction is often used to describe virus-mediated transfer of nucleic acid molecules or polynucleotides. Transfection of animal cells typically involves opening transient pores or “holes” in the cell membrane, to allow the uptake of material. Transfection can be carried out using biological particles (such as viral transfection, also called viral transduction), chemical-based methods (such as using calcium phosphate, lipofection, Fugene, cationic polymers, nanoparticles) or physical treatment (such as electroporation, microinjection, gene gun, cell squeezing, magnetofection, hydrostatic pressure, impalefection, sonication, optical transfection, heat shock).

The term “transformation” is used to describe non-viral transfer of nucleic acid molecules or polynucleotides (including vectors) into bacteria, and into non-animal eukaryotic cells, including plant cells. Transformation is hence the genetic alteration of a bacterial or non-animal eukaryotic cell resulting from the direct uptake through the cell membrane(s) from its surroundings and subsequent incorporation of exogenous genetic material (nucleic acid molecules). Transformation can be achieved by artificial means. For transformation to happen, cells or bacteria must be in a state of competence, which might occur as a time-limited response to environmental conditions such as starvation and cell density and can also be artificially induced.

Moreover, the invention provides a host cell transformed or transfected with the polynucleotide/nucleic acid molecule of the invention or with the vector of the invention.

As used herein, the terms “host cell” or “recipient cell” are intended to include any individual cell or cell culture that can be or has been recipient of vectors, exogenous nucleic acid molecules and/or polynucleotides encoding the construct of the present invention; and/or recipients of the construct itself. The introduction of the respective material into the cell is carried out by way of transformation, transfection and the like (vide supra). The term “host cell” is also intended to include progeny or potential progeny of a single cell. Because certain modifications may occur in succeeding generations due to either natural, accidental, or deliberate mutation or due to environmental influences, such progeny may not, in fact, be completely identical (in morphology or in genomic or total DNA complement) to the parent cell but is still included within the scope of the term as used herein. Suitable host cells include prokaryotic or eukaryotic cells and include—but are not limited to—bacteria (such as E. coli), yeast cells, fungi cells, plant cells, and animal cells such as insect cells and mammalian cells, e.g., hamster, murine, rat, macaque or human.

In addition to prokaryotes, eukaryotic microbes such as filamentous fungi or yeast are suitable cloning or expression hosts for the construct of the invention. Saccharomyces cerevisiae, or common baker's yeast, is the most commonly used among lower eukaryotic host microorganisms. However, a number of other genera, species, and strains are commonly available and useful herein, such as Schizosaccharomyces pombe, Kluyveromyces hosts such as K. lactis, K. fragilis (ATCC 12424), K. bulgaricus (ATCC 16045), K. wickeramii (ATCC 24178), K. waltii (ATCC 56500), K. drosophilarum (ATCC 36906), K. thermotolerans, and K. marxianus; yarrowia (EP 402 226); Pichia pastoris (EP 183 070); Candida; Trichoderma reesia (EP 244 234); Neurospora crassa; Schwanniomyces such as Schwanniomyces occidentalis; and filamentous fungi such as Neurospora, Penicillium, Tolypocladium, and Aspergillus hosts such as A. nidulans and A. niger.

Suitable host cells for the expression of a glycosylated construct are derived from multicellular organisms. Examples of invertebrate cells include plant and insect cells. Numerous baculoviral strains and variants and corresponding permissive insect host cells from hosts such as Spodoptera frugiperda (caterpillar), Aedes aegypti (mosquito), Aedes albopictus (mosquito), Drosophila melanogaster (fruit fly), and Bombyx mori (silkmoth) have been identified. A variety of viral strains for transfection are publicly available, e.g., the L-1 variant of Autographa californica NPV and the Bm-5 strain of Bombyx mori NPV, and such viruses may be used as the virus herein according to the present invention, particularly for transfection of Spodoptera frugiperda cells.

Plant cell cultures of cotton, corn, potato, soybean, petunia, tomato, Arabidopsis and tobacco can also be used as hosts. Cloning and expression vectors useful in the production of proteins in plant cell culture are known to those of skill in the art. See e.g. Hiatt et al., Nature (1989) 342: 76-78, Owen et al. (1992) Bio/Technology 10: 790-794, Artsaenko et al. (1995) The Plant J 8: 745-750, and Fecker et al. (1996) Plant Mol Biol 32: 979-986.

However, interest has been greatest in vertebrate cells, and propagation of vertebrate cells in culture (cell culture) has become a routine procedure. Examples of useful mammalian host cell lines are monkey kidney CV1 line transformed by SV40 (such as COS-7, ATCC CRL 1651); human embryonic kidney line (such as 293 or 293 cells subcloned for growth in suspension culture, Graham et al., J. Gen Virol. 36: 59 (1977)); baby hamster kidney cells (such as BHK, ATCC CCL 10); Chinese hamster ovary cells/-DHFR (such as CHO, Urlaub et al., Proc. Natl. Acad. Sci. USA 77: 4216 (1980)); mouse sertoli cells (such as TM4, Mather, Biol. Reprod. 23: 243-251 (1980)); monkey kidney cells (such as CVI ATCC CCL 70); African green monkey kidney cells (such as VERO-76, ATCC CRL1587); human cervical carcinoma cells (such as HELA, ATCC CCL 2); canine kidney cells (such as MDCK, ATCC CCL 34); buffalo rat liver cells (such as BRL 3A, ATCC CRL 1442); human lung cells (such as W138, ATCC CCL 75); human liver cells (such as Hep G2,1413 8065); mouse mammary tumor (such as MMT 060562, ATCC CCL-51); TRI cells (Mather et al., Annals N. Y Acad. Sci. (1982) 383: 44-68); MRC 5 cells; FS4 cells; and a human hepatoma line (such as Hep G2).

In a further embodiment, the invention provides a process for the production of a polypeptide or polypeptide construct of the invention, said process comprising culturing a host cell of the invention under conditions allowing the expression of the construct of the invention and recovering the produced construct from the culture.

As used herein, the term “culturing” refers to the in vitro maintenance, differentiation, growth, proliferation and/or propagation of cells under suitable conditions in a medium. Cells are grown and maintained in a cell growth medium at an appropriate temperature and gas mixture. Culture conditions vary widely for each cell type. Typical growth conditions are a temperature of about 37° C., a C02 concentration of about 5% and a humidity of about 95%. Recipes for growth media can vary e.g. in pH, concentration of the carbon source (such as glucose), nature and concentration of growth factors, and the presence of other nutrients (such as amino acids or vitamins). The growth factors used to supplement media are often derived from the serum of animal blood, such as fetal bovine serum (FBS), bovine calf serum (FCS), equine serum, and porcine serum. Cells can be grown either in suspension or as adherent cultures. There are also cell lines that have been modified to be able to survive in suspension cultures, so they can be grown to a higher density than adherent conditions would allow.

The term “expression” includes any step involved in the production of a construct of the invention including, but not limited to, transcription, post-transcriptional modification, translation, folding, post-translational modification, targeting to specific subcellular or extracellular locations, and secretion. The term “recovering” refers to a series of processes intended to isolate the construct from the cell culture. The “recovering” or “purification” process may separate the protein and non-protein parts of the cell culture, and finally separate the desired construct from all other polypeptides and proteins. Separation steps usually exploit differences in protein size, physico-chemical properties, binding affinity and biological activity. Preparative purifications aim to produce a relatively large quantity of purified proteins for subsequent use, while analytical purification produces a relatively small amount of a protein for a variety of research or analytical purposes.

When using recombinant techniques, the construct can be produced intracellularly, in the periplasmic space, or directly secreted into the medium. If the construct is produced intracellularly, as a first step, the particulate debris, either host cells or lysed fragments, are removed, for example, by centrifugation or ultrafiltration. The construct of the invention may e.g. be produced in bacteria such as E. coli. After expression, the construct is isolated from the bacterial cell paste in a soluble fraction and can be purified e.g. via affinity chromatography and/or size exclusion. Final purification can be carried out in a manner that is like the process for purifying a construct expressed in mammalian cells and secreted into the medium. Carteret al. (Biotechnology (NY) 1992 February; 10(2):163-7) describe a procedure for isolating antibodies which are secreted to the periplasmic space of E. coli.

Where the antibody is secreted into the medium, supernatants from such expression systems are generally first concentrated using a commercially available protein concentration filter, for example, an ultrafiltration unit.

The construct of the invention prepared from the host cells can be recovered or purified using, for example, hydroxylapatite chromatography, gel electrophoresis, dialysis, and affinity chromatography. Other techniques for protein purification such as fractionation on an ion-exchange column, mixed mode ion exchange, HIC, ethanol precipitation, size exclusion chromatography, reverse phase HPLC, chromatography on silica, chromatography on heparin sepharose, chromatography on an anion or cation exchange resin (such as a polyaspartic acid column), immunoaffinity (such as Protein A/G/L) chromatography, chromato-focusing, SDS-PAGE, ultracentrifugation, and ammonium sulfate precipitation are also available depending on the construct to be recovered.

A protease inhibitor may be included in any of the foregoing steps to inhibit proteolysis, and antibiotics may be included to prevent the growth of contaminants.

Moreover, the invention provides a pharmaceutical composition or formulation comprising a polypeptide or polypeptide construct of the invention or a polypeptide or polypeptide construct produced according to the process of the invention.

As used herein, the term “pharmaceutical composition” relates to a composition which is suitable for administration to a patient, preferably a human patient. The particularly preferred pharmaceutical composition of this invention comprises one or a plurality of the construct(s) of the invention, preferably in a therapeutically effective amount. Preferably, the pharmaceutical composition further comprises suitable formulations of one or more (pharmaceutically effective) carriers, stabilizers, excipients, diluents, solubilizers, surfactants, emulsifiers, preservatives and/or adjuvants. Acceptable constituents of the composition are preferably nontoxic to recipients at the dosages and concentrations employed. Pharmaceutical compositions of the invention include, but are not limited to, liquid, frozen, and lyophilized compositions.

The compositions may comprise a pharmaceutically acceptable carrier. In general, as used herein, “pharmaceutically acceptable carrier” means all aqueous and non-aqueous solutions, sterile solutions, solvents, buffers, e.g. phosphate buffered saline (PBS) solutions, water, suspensions, emulsions, such as oil/water emulsions, various types of wetting agents, liposomes, dispersion media and coatings, which are compatible with pharmaceutical administration, in particular with parenteral administration. The use of such media and agents in pharmaceutical compositions is well known in the art, and the compositions comprising such carriers can be formulated by well-known conventional methods.

Certain embodiments provide pharmaceutical compositions comprising the construct of the invention and further one or more excipients such as those illustratively described in this section and elsewhere herein. Excipients can be used in the invention for a wide variety of purposes, such as adjusting physical, chemical, or biological properties of formulations, such as adjustment of viscosity, and or processes of the invention to improve effectiveness and/or to stabilize such formulations and processes against degradation and spoilage e.g. due to stresses that occur during manufacturing, shipping, storage, pre-use preparation, administration, and thereafter. Excipients should in general be used in their lowest effective concentrations.

In certain embodiments, the pharmaceutical composition may contain formulation materials for modifying, maintaining or preserving certain characteristics of the composition such as the pH, osmolarity, viscosity, clarity, color, isotonicity, odor, sterility, stability, rate of dissolution or release, adsorption or penetration (see, Remington's Pharmaceutical Sciences, 18” Edition, 1990, Mack Publishing Company). In such embodiments, suitable formulation materials may include, but are not limited to:
amino acids
antimicrobials such as antibacterial and antifungal agents
antioxidants
buffers, buffer systems and buffering agents that are used to maintain the composition at physiological pH or at a slightly lower pH, typically within a range of from about 5 to about 8 or 9
non-aqueous solvents, vegetable oils, and injectable organic esters
aqueous carriers including water, alcoholic/aqueous solutions, emulsions or suspensions, including saline and buffered media
biodegradable polymers such as polyesters
bulking agents
chelating agents
isotonic and absorption delaying agents
complexing agents
fillers
carbohydrates
(low molecular weight) proteins, polypeptides or proteinaceous carriers, preferably of human origin
coloring and flavouring agents
sulfur containing reducing agents
diluting agents
emulsifying agents
hydrophilic polymers
salt-forming counter-ions
preservatives
metal complexes
solvents and co-solvents
sugars and sugar alcohols
suspending agents
surfactants or wetting agents
stability enhancing agents
tonicity enhancing agents
parenteral delivery vehicles
intravenous delivery vehicles

It is common knowledge that the different constituents of the pharmaceutical composition can have different effects, for example, and amino acid can act as a buffer, a stabilizer and/or an antioxidant; mannitol can act as a bulking agent and/or a tonicity enhancing agent; sodium chloride can act as delivery vehicle and/or tonicity enhancing agent; etc.

In the context of the present invention, a pharmaceutical composition may comprise:
(a) a polypeptide or polypeptide construct as described herein,
(b) at least one buffer agent,
(c) at least one saccharide, and
(d) at least one surfactant;
wherein the pH of the pharmaceutical composition is in the range of 3.5 to 6.

In the composition described above, the first domain preferably has an isoelectric point (pI) in the range of 4 to 9.5; the second domain has a pI in the range of 8 to 10, preferably 8.5 to 9.0; and the construct optionally comprises a third domain comprising two polypeptide monomers, each comprising a hinge, a CH2 domain and a CH3 domain, wherein said two polypeptide monomers are fused to each other via a peptide linker.

In the composition described above, it is further envisaged that the at least one buffer agent is present at a concentration range of 5 to 200 mM, more preferably at a concentration range of 10 to 50 mM. It is also envisaged that the at least one saccharide is selected from the group consisting of monosaccharide, disaccharide, cyclic polysaccharide, sugar alcohol, linear branched dextran or linear non-branched dextran. It is also envisaged that the disaccharide is selected from the group consisting of sucrose, trehalose and mannitol, sorbitol, and combinations thereof. It is further envisaged that the sugar alcohol is sorbitol. It is also envisaged that the at least one saccharide is present at a concentration in the range of 1 to 15% (m/V), preferably in a concentration range of 9 to 12% (m/V). It is further envisaged that the construct is present in a concentration range of 0.1 to 8 mg/ml, preferably of 0.2-2.5 mg/ml, more preferably of 0.25-1.0 mg/ml.

According to one embodiment of the composition described above, the at least one surfactant is selected from the group consisting of polysorbate 20, polysorbate 40, polysorbate 60, polysorbate 80, poloxamer 188, pluronic F68, triton X-100, polyoxyethylen, PEG 3350, PEG 4000 and combinations thereof. It is further envisaged that the at least one surfactant is present at a concentration in the range of 0.004 to 0.5% (m/V), preferably in the range of 0.001 to 0.01% (m/V). It is envisaged that the pH of the composition is in the range of 4.0 to 5.0, preferably 4.2. It is also envisaged that the pharmaceutical composition has an osmolarity in the range of 150 to 500 mOsm. It is further envisaged that the pharmaceutical composition further comprises an excipient selected from the group consisting of one or more polyol(s) and one or more amino acid(s). It is envisaged in the context of the present invention that said one or more excipient is present in the concentration range of 0.1 to 15% (w/V).

The present invention also provides a pharmaceutical composition comprising (a) the construct as described herein, preferably in a concentration range of 0.1 to 8 mg/ml, preferably of 0.2-2.5 mg/ml, more preferably of 0.25-1.0 mg/ml; (b) 10 mM glutamate or acetate; (c) 9% (m/V) sucrose or 6% (m/V) sucrose and 6% (m/V) hydroxypropyl-β-cyclodextrin; (d) 0.01% (m/V) polysorbate 80; wherein the pH of the liquid pharmaceutical composition is 4.2.

It is envisaged that the composition of the invention might comprise, in addition to the construct of the invention defined herein, further biologically active agents, depending on the intended use of the composition. Such agents might be drugs acting on the gastro-intestinal system, drugs acting as cytostatica, drugs preventing hyperurikemia, drugs inhibiting immunoreactions, drugs modulating the inflammatory response, drugs acting on the circulatory system and/or agents such as cytokines known in the art. It is also envisaged that the polypeptide construct of the present invention is applied in a co-therapy, i.e., in combination with another anti-cancer medicament.

In this context, it is envisaged that the pharmaceutical composition of the invention (which comprises a construct comprising a CD3 binding domain and at least a further binding domain which binds to a cell surface target antigen, preferably a tumor antigen on the surface of a target cell, as described in more detail herein above) furthermore comprises an agent, preferably an antibody or construct, which binds to a protein of the immune checkpoint pathway (such as PD-1 or CTLA-4) or to a co-stimulatory immune checkpoint receptor (such as 4-11BE). The present invention also refers to a combination of a polypeptide construct according to the invention (which comprises a construct comprising a CD3 binding domain and at least a further binding domain which binds to a cell surface target antigen, preferably a tumor antigen on the surface of a target cell, as described in more detail herein above) and an agent, preferably an antibody or polypeptide construct, which binds to a protein of the immune checkpoint pathway (such as PD-1 or CTLA-4) or to a co-stimulatory immune checkpoint receptor (such as 4-11BE). Due to the nature of the at least two ingredients of the combination, namely their pharmaceutical activity, the combination can also be referred to as a therapeutic combination. In some embodiments, the combination can be in the form of a pharmaceutical composition or of a kit. According to one embodiment, the pharmaceutical composition or the combination comprises a construct of the invention and an antibody or construct which binds to PD-1. Anti-PD-1 binding proteins useful for this purpose are e.g. described in detail in PCT/US2019/013205 incorporated herein by reference.

In certain embodiments, the optimal pharmaceutical composition is determined depending upon, for example, the intended route of administration, delivery format and desired dosage. See, for example, Remington's Pharmaceutical Sciences, supra. In certain embodiments, such compositions may influence the physical state, stability, rate of in vivo release and rate of in vivo clearance of the construct of the invention. In certain embodiments, the primary vehicle or carrier in a pharmaceutical composition may be either aqueous or non-aqueous in nature. For example, a suitable vehicle or carrier may be water for injection or physiological saline solution, possibly supplemented with other materials common in compositions for parenteral administration. In certain embodiments, the compositions comprising the construct of the invention may be prepared for storage by mixing the selected composition having the desired degree of purity with optional formulation agents (Remington's Pharmaceutical Sciences, supra) in the form of a lyophilized cake or an aqueous solution. Further, in certain embodiments, the construct of the invention may be formulated as a lyophilizate using appropriate excipients.

When parenteral administration is contemplated, the therapeutic compositions for use in this invention may be provided in the form of a pyrogen-free, parenterally acceptable aqueous solution comprising the desired construct of the invention in a pharmaceutically acceptable vehicle. A particularly suitable vehicle for parenteral injection is sterile distilled water in which the construct of the invention is formulated as a sterile, isotonic solution, properly preserved. In certain embodiments, the preparation can involve the formulation of the desired molecule with an agent that may provide controlled or sustained release of the product which can be delivered via depot injection, or that may promote sustained duration in the circulation. In certain embodiments, implantable drug delivery devices may be used to introduce the desired construct.

Additional pharmaceutical compositions will be evident to those skilled in the art, including formulations involving the construct of the invention in sustained or controlled delivery formulations. Techniques for formulating a variety of sustained- or controlled-delivery means are known to those skilled in the art. The construct may also be entrapped in microcapsules prepared, for example, by coacervation techniques or by interfacial polymerization, in colloidal drug delivery systems, or in macroemulsions. Such techniques are disclosed in Remington's Pharmaceutical Sciences, supra.

Pharmaceutical compositions used for in vivo administration are typically provided as sterile preparations. Sterilization can be accomplished by filtration through sterile filtration membranes. When the composition is lyophilized, sterilization using this method may be conducted either prior to or following lyophilization and reconstitution. Compositions for parenteral administration can be stored in lyophilized form or in a solution. Parenteral compositions are generally placed into a container having a sterile access port, for example, an intravenous solution bag or vial having a stopper pierceable by a hypodermic injection needle.

Another aspect of the invention includes self-buffering formulations comprising the construct of the invention, which can be used as pharmaceutical compositions, as described in international patent application WO 2006/138181. A variety of publications are available on protein stabilization and formulation materials and methods useful in this regard, such as Arawaka T. et al., Pharm Res. 1991 March; 8(3):285-91; Kendrick et al., “Physical stabilization of proteins in aqueous solution” in: Rational Design of Stable Protein Formulations: Theory and Practice, Carpenter and Manning, eds. Pharmaceutical Biotechnology. 13: 61-84 (2002), and Randolph and Jones, Pharm Biotechnol. 2002; 13:159-75, see particularly the parts pertinent to excipients and processes for self-buffering protein formulations, especially as to protein pharmaceutical products and processes for veterinary and/or human medical uses.

Salts may be used in accordance with certain embodiments of the invention, e.g. to adjust the ionic strength and/or the isotonicity of a composition or formulation and/or to improve the solubility and/or physical stability of a construct or other ingredient of a composition in accordance with the invention. Ions can stabilize the native state of proteins by binding to charged residues on the protein's surface and by shielding charged and polar groups in the protein and reducing the strength of their electrostatic interactions, attractive, and repulsive interactions. Ions also can stabilize the denatured state of a protein by binding to, particularly the denatured peptide linkages (—CONH) of the protein. Furthermore, ionic interaction with charged and polar groups in a protein also can reduce intermolecular electrostatic interactions and, thereby, prevent or reduce protein aggregation and insolubility.

Ionic species differ significantly in their effects on proteins. Several categorical rankings of ions and their effects on proteins have been developed that can be used in formulating pharmaceutical compositions in accordance with the invention. One example is the Hofmeister series, which ranks ionic and polar non-ionic solutes by their effect on the conformational stability of proteins in solution. Stabilizing solutes are referred to as “kosmotropic”. Destabilizing solutes are referred to as “chaotropic”. Kosmotropes are commonly used at high concentrations to precipitate proteins from solution (“salting-out”). Chaotropes are commonly used to denature and/or to solubilize proteins (“salting-in”). The relative effectiveness of ions to “salt-in” and “salt-out” defines their position in the Hofmeister series.

Free amino acids can be used in formulations or compositions comprising the construct of the invention in accordance with various embodiments of the invention as bulking agents, stabilizers, and antioxidants, as well as for other standard uses. Certain amino acids can be used for stabilizing proteins in a formulation, others are useful during lyophilization to ensure correct cake structure and properties of the active ingredient. Some amino acids may be useful to inhibit protein aggregation in both liquid and lyophilized formulations, and others are useful as antioxidants.

Polyols are kosmotropic and are useful as stabilizing agents in both liquid and lyophilized formulations to protect proteins from physical and chemical degradation processes. Polyols are also useful for adjusting the tonicity of formulations and for protecting against freeze-thaw stresses during transport or the preparation of bulks during the manufacturing process. Polyols can also serve as cryoprotectants in the context of the present invention.

Certain embodiments of the formulation or composition comprising the construct of the invention can comprise surfactants. Proteins may be susceptible to adsorption on surfaces and to denaturation and resulting aggregation at air-liquid, solid-liquid, and liquid-liquid interfaces. These deleterious interactions generally scale inversely with protein concentration and are typically exacerbated by physical agitation, such as that generated during the shipping and handling of a product. Surfactants are routinely used to prevent, minimize, or reduce surface adsorption. Surfactants also are commonly used to control protein conformational stability. The use of surfactants in this regard is protein specific, since one specific surfactant will typically stabilize some proteins and destabilize others.

Certain embodiments of the formulation or composition comprising the construct of the invention can comprise one or more antioxidants. To some extent deleterious oxidation of proteins can be prevented in pharmaceutical formulations by maintaining proper levels of ambient oxygen and temperature and by avoiding exposure to light. Antioxidant excipients can also be used to prevent oxidative degradation of proteins. It is envisaged that antioxidants for use in therapeutic protein formulations in accordance with the present invention can be water-soluble and maintain their activity throughout the shelf life of the product (the composition comprising the construct). Antioxidants can also damage proteins and should hence—among other things—be selected in a way to eliminate or sufficiently reduce the possibility of antioxidants damaging the construct or other proteins in the formulation.

Certain embodiments of the formulation or composition comprising the construct of the invention can comprise one or more preservatives. Preservatives are necessary for example when developing multi-dose parenteral formulations that involve more than one extraction from the same container. Their primary function is to inhibit microbial growth and ensure product sterility throughout the shelf-life or term of use of the drug product. Although preservatives have a long history of use with small-molecule parenterals, the development of protein formulations that include preservatives can be challenging. Preservatives very often have a destabilizing effect (aggregation) on proteins, and this has become a major factor in limiting their use in multi-dose protein formulations. To date, most protein drugs have been formulated for single-use only. However, when multi-dose formulations are possible, they have the added advantage of enabling patient convenience, and increased marketability. A good example is that of human growth hormone (hGH) where the development of preserved formulations has led to commercialization of more convenient, multi-use injection pen presentations. Several aspects need to be considered during the formulation and development of preserved dosage forms. The effective preservative concentration in the drug product must be optimized. This requires testing a given preservative in the dosage form with concentration ranges that confer anti-microbial effectiveness without compromising protein stability.

As might be expected, development of liquid formulations containing preservatives are more challenging than lyophilized formulations. Freeze-dried products can be lyophilized without the preservative and reconstituted with a preservative containing diluent at the time of use. This shortens the time during which a preservative is in contact with the construct, significantly minimizing the associated stability risks. With liquid formulations, preservative effectiveness and stability should be maintained over the entire product shelf-life. An important point to note is that preservative effectiveness should be demonstrated in the final formulation containing the active drug and all excipient components. Once the pharmaceutical composition has been formulated, it may be stored in sterile vials as a solution, suspension, gel, emulsion, solid, crystal, or as a dehydrated or lyophilized powder. Such formulations may be stored either in a ready-to-use form or in a form (e.g., lyophilized) that is reconstituted prior to administration.

The biological activity of the pharmaceutical composition defined herein can be determined for instance by in vitro cytotoxicity assays, as described in the following examples, in WO 99/54440 or by Schlereth et al. (Cancer Immunol. Immunother. 20 (2005), 1-12). “Efficacy” or “in vivo efficacy” as used herein refers to the response to therapy by the pharmaceutical composition of formulation of the invention, using e.g. standardized NCI response criteria. The success or in vivo efficacy of the therapy using a pharmaceutical composition of the invention refers to the effectiveness of the composition for its intended purpose, i.e. the ability of the composition to cause its desired effect, i.e. depletion of pathologic cells, e.g. tumor cells. The in vivo efficacy may be monitored by established standard methods for the respective disease entities including, but not limited to, white blood cell counts, differentials, fluorescence activated cell sorting, bone marrow aspiration. In addition, various disease specific clinical chemistry parameters and other established standard methods may be used. Furthermore, computer-aided tomography, X-ray, nuclear magnetic resonance tomography, positron-emission tomography scanning, lymph node biopsies/histologies and other established standard methods may be used.

Another major challenge in the development of drugs such as the pharmaceutical composition of the invention is the predictable modulation of pharmacokinetic properties. To this end, a pharmacokinetic profile of the drug candidate, i.e. a profile of the pharmacokinetic parameters that affect the ability of a specific drug to treat a given condition, can be established. Pharmacokinetic parameters of the drug influencing the ability of a drug for treating a certain disease entity include, but are not limited to: half-life, volume of distribution, hepatic first-pass metabolism and the degree of blood serum binding. The efficacy of a given drug agent can be influenced by each of the parameters mentioned above.

“Half-life” is the time required for a quantity to reduce to half its initial value. The medical sciences refer to the half-life of substances or drugs in the human body. In a medical context, half-life may refer to the time it takes for a substance/drug to lose one-half of its activity, e.g. pharmacologic, physiologic, or radiological activity. The half-life may also describe the time that it takes for the concentration of a drug or substance (e.g., a construct of the invention) in blood plasma/serum to reach one-half of its steady-state value (“serum half-life”). Typically, the elimination or removal of an administered substance/drug refers to the body's cleansing through biological processes such as metabolism, excretion, also involving the function of kidneys and liver. The “first-pass metabolism” is a phenomenon of drug metabolism whereby the concentration of a drug is reduced before it reaches the circulation. It is the fraction of drug lost during the process of absorption. Accordingly, by “hepatic first-pass metabolism” is meant the propensity of a drug to be metabolized upon first contact with the liver, i.e. during its first pass through the liver. “Volume of distribution” (VD) means the degree to which a drug is distributed in body tissue rather than the blood plasma, a higher VD indicating a greater amount of tissue distribution. The retention of a drug can occur throughout the various compartments of the body, such as intracellular and extracellular spaces, tissues and organs, etc. “Degree of blood serum binding” means the propensity of a drug to interact with and bind to blood serum proteins, such as albumin, leading to a reduction or loss of biological activity of the drug.

Pharmacokinetic parameters also include bioavailability, lag time (T lag), Tmax, absorption rates, and/or Cmax for a given amount of drug administered. “Bioavailability” refers to the fraction of an administered dose of a drug/substance that reaches the systemic circulation (the blood compartment). When a medication is administered intravenously, its bioavailability is considered to be 100%. However, when a medication is administered via other routes (such as orally), its bioavailability generally decreases. “Lag time” means the time delay between the administration of the drug and its detection and measurability in blood or plasma. Cmax is the maximum plasma concentration that a drug achieves after its administration (and before the administration of a second dose). Tmax is the time at which Cmax is reached. The time to reach a blood or tissue concentration of the drug which is required for its biological effect is influenced by all parameters. Pharmacokinetic parameters of constructs exhibiting cross-species specificity may be determined in preclinical animal testing in non-chimpanzee primates as outlined above and set forth e.g. in Schlereth et al. (supra).

One embodiment provides the construct of the invention (or the construct produced according to the process of the invention), for the use as a medicament, particularly for the use in the prevention, treatment or amelioration (preferably treatment) of a disease, preferably a tumorous disease, more preferred a neoplasm, cancer or tumor. Another embodiment provides the use of the construct of the invention (or of the construct produced according to the process of the invention) in the manufacture of a medicament for the prevention, treatment or amelioration of a disease, preferably a tumorous disease, more preferred a neoplasm, cancer or tumor. It is also envisaged to provide a method for the prevention, treatment or amelioration of a disease, preferably a tumorous disease, more preferred a neoplasm, cancer or tumor, comprising the step of administering to a subject in need thereof the construct of the present invention (or the construct produced according to the process of the present invention). The terms “subject in need”, “patient” or those “in need of treatment” include those already with the disease, as well as those in which the disease is to be prevented. The terms also include human and other mammalian subjects that receive either prophylactic or therapeutic treatment.

The polypeptides/polypeptide constructs of the invention and the formulations/pharmaceutical compositions described herein are useful in the treatment, amelioration and/or prevention of the medical condition as described herein in a patient in need thereof. The term “treatment” refers to both therapeutic treatment and prophylactic or preventative measures. Treatment includes the application or administration of the polypeptides/polypeptide constructs/pharmaceutical composition to the body, to an isolated tissue, or to a cell from a patient or a subject in need who has a disease/disorder as described herein, a symptom of such disease/disorder, or a predisposition toward such disease/disorder, with the purpose to cure, heal, alleviate, relieve, alter, remedy, ameliorate, improve, or affect the disease, the symptom of the disease, or the predisposition toward the disease. The term “amelioration” as used herein refers to any improvement of the disease state of a patient, by the administration of a polypeptide construct according to the invention to such patient or subject in need thereof. Such an improvement may be a slowing down or stopping of the progression of the disease of the patient, and/or as a decrease in severity of disease symptoms, an increase in frequency or duration of disease symptom-free periods or a prevention of impairment or disability due to the disease. The term “prevention” as used herein means the avoidance of the occurrence or of the re-occurrence of a disease as specified herein, by the administration of a construct according to the invention to a subject in need thereof.

The term “disease” refers to any condition that would benefit from treatment with the construct or the pharmaceutical composition described herein. This includes chronic and acute disorders or diseases including those pathological conditions that predispose the mammal to the disease in question. The disease is preferably a tumorous disease, more preferred a neoplasm, cancer or tumor. The disease, neoplasm, cancer or tumor is preferably positive for a tumor antigen, preferably such as those defined herein above, i.e. it is characterized by expression or overexpression of a tumor antigen, preferably such as those defined herein above. An overexpression of a tumor antigen means that there is an increase by at least 10%, in particular at least 25%, at least 50%, at least 100%, at least 250%, at least 500%, at least 750%, at least 1000% or even more. Expression is, preferably, only found in a diseased tissue, while expression in a corresponding healthy tissue is not or significantly not detectable. According to the invention, diseases associated with cells expressing a tumor antigen, preferably such as those defined herein above, include cancer diseases. Furthermore, according to the invention, cancer diseases preferably are those wherein the cancer cells express a tumor antigen. In accordance with the invention, the disease, preferably tumorous disease, more preferred neoplasm, tumor or cancer is preferably characterized by the presence of BCMA-positive, CD123-positive, CD19-positive, CD20-positive, CD22-positive, CD33-positive, CD70-positive, CDH19-positive, CDH3-positive, CLL1-positive, CS1-positive, CLDN6-positive, CLDN18.2-positive, DLL3-positive, EGFRvIII-positive, FLT3-positive, MAGEB2-positive, MART1-positive, MSLN-positive, MUC17-positive, PSMA-positive, or STEAP1-positive cells. In other words, the tumorous disease, more preferred neoplasm, tumor or cancer is preferably associated with the presence of BCMA-positive, CD123-positive, CD19-positive, CD20-positive, CD22-positive, CD33-positive, CD70-positive, CDH19-positive, CDH3-positive, CLL1-positive, CS1-positive, CLDN6-positive, CLDN18.2-positive, DLL3-positive, EGFRvIII-positive, FLT3-positive, MAGEB2-positive, MART1-positive, MSLN-positive, MUC17-positive, PSMA-positive, or STEAP1-positive cells; the tumorous disease, more preferred neoplasm, tumor or cancer can therefore be termed a BCMA-positive, CD123-positive, CD19-positive, CD20-positive, CD22-positive, CD33-positive, CD70-positive, CDH19-positive, CDH3-positive, CLL1-positive, CS1-positive, CLDN6-positive, CLDN18.2-positive, DLL3-positive, EGFRvIII-positive, FLT3-positive, MAGEB2-positive, MART1-positive, MSLN-positive, MUC17-positive, PSMA-positive, or STEAP1-positive neoplasm, tumor or cancer. It is understood herein, that each of said tumor antigen-positive neoplasms, tumors or cancers can be prevented, treated or ameliorated using a polypeptide or polypeptide construct according to the invention that comprises a binding domain against the tumor antigen expressed by the cells with which said neoplasm, tumor or cancer is associated with. A BCMA-positive, CD123-positive, CD19-positive, CD20-positive, CD22-positive, CD33-positive, CD70-positive, CDH19-positive, CDH3-positive, CLL1-positive, CS1-positive, CLDN6-positive, CLDN18.2-positive, DLL3-positive, EGFRvII-positive, FLT3-positive, MAGEB2-positive, MART1-positive, MSLN-positive, MUC17-positive, PSMA-positive, or STEAP1-positive neoplasm, tumor or cancer can be prevented, treated or ameliorated using a polypeptide or polypeptide construct according to the invention that comprises a binding domain against BCMA (for a BCMA-positive neoplasm, tumor or cancer), CD123 (for a CD123-positive neoplasm, tumor or cancer), CD19 (for a CD19-positive neoplasm, tumor or cancer), CD20 (for a CD20-positive neoplasm, tumor or cancer), CD22 (for a CD22-positive neoplasm, tumor or cancer), CD33 (for a CD33-positive neoplasm, tumor or cancer), CD70 (for a CD70-positive neoplasm, tumor or cancer), CDH19 (for a CDH19-positive neoplasm, tumor or cancer), CDH3 (for a CDH3-positive neoplasm, tumor or cancer), CLL1 (for a CLL1-positive neoplasm, tumor or cancer), CS1 (for a CS1-positive neoplasm, tumor or cancer), CLDN6 (for a CLDN6-positive neoplasm, tumor or cancer), CLDN18.2 (for a CLDN18.2-positive neoplasm, tumor or cancer), DLL3 (for a DLL3-positive neoplasm, tumor or cancer), EGFRvIII (for a EGFRvIII-positive neoplasm, tumor or cancer), FLT3 (for a FLT3-positive neoplasm, tumor or cancer), MAGEB2 (for a MAGEB2-positive neoplasm, tumor or cancer), MART1 (for a MART1-positive neoplasm, tumor or cancer), MSLN (for a MSLN-positive neoplasm, tumor or cancer), MUC17 (for a MUC17-positive neoplasm, tumor or cancer), PSMA (for a PSMA-positive neoplasm, tumor or cancer), and STEAP1 (for a STEAP1-positive neoplasm, tumor or cancer), respectively.

A “neoplasm” is an abnormal growth of tissue, usually but not always forming a mass. When also forming a mass, it is commonly referred to as a “tumor”. Neoplasms or tumors can be benign, potentially malignant (pre-cancerous), or malignant (cancerous). Malignant neoplasms/tumors are commonly called cancer. They usually invade and destroy the surrounding tissue and may form metastases, i.e., they spread to other parts, tissues or organs of the body. A “primary tumor” is a tumor growing at the anatomical site where tumor progression began and proceeded to yield a cancerous mass. Most cancers develop at their primary site but then go on to metastasize or spread to other parts (e.g. tissues and organs) of the body. These further tumors are “secondary tumors”. Most cancers continue to be called after their primary site, even after they have spread to other parts of the body.

Lymphomas and leukemias are lymphoid neoplasms. For the purposes of the present invention, they are also encompassed by the terms “tumor” and “cancer”. For the purposes of the present invention, the terms “neoplasm”, “tumor” and “cancer” may be used interchangeably, and they comprise both primary tumors/cancers and secondary tumors/cancers (or “metastases”) as well as mass-forming neoplasms (tumors) and lymphoid neoplasms (such as lymphomas and leukemias), and minimal residual disease (MRD).

The term “minimal residual disease” (MRD) refers to the evidence for the presence of small numbers of residual cancer cells that remain in the patient after cancer treatment, e.g. when the patient is in remission (no symptoms or signs of disease). A very small number of remaining cancer cells usually cannot be detected by routine means because the standard tests used to assess or detect cancer are not sensitive enough to detect MRD. Nowadays, very sensitive molecular biology tests for MRD are available, such as flow cytometry, PCR and next-generation sequencing. These tests can measure minimal levels of cancer cells in tissue samples, sometimes as low as one cancer cell in a million normal cells. In the context of the present invention, the terms “prevention”, “treatment” or “amelioration” of a cancer are envisaged to also encompass “prevention, treatment or amelioration of MRD”, whether the MRD was detected or not.

The construct of the invention will generally be designed for specific routes and methods of administration, for specific dosages and frequencies of administration, for specific treatments of specific diseases, with ranges of bio-availability and persistence, among other things. The materials of the composition are preferably formulated in concentrations that are acceptable for the site of administration. Formulations and compositions thus may be designed in accordance with the invention for delivery by any suitable route of administration. In the context of the present invention, the routes of administration include, but are not limited to topical routes, enteral routes and parenteral routes.

If the pharmaceutical composition has been lyophilized, the lyophilized material is first reconstituted in an appropriate liquid prior to administration. The lyophilized material may be reconstituted in, e.g., bacteriostatic water for injection (BWFI), physiological saline, phosphate buffered saline (PBS), or the same formulation the protein had been in prior to lyophilization. The pharmaceutical compositions and the construct of this invention are particularly useful for parenteral administration, e.g., intravenous delivery, for example by injection or infusion. Pharmaceutical compositions may be administered using a medical device. Examples of medical devices for administering pharmaceutical compositions are described in U.S. Pat. Nos. 4,475,196; 4,439,196; 4,447,224; 4,447,233; 4,486,194; 4,487,603; 4,596,556; 4,790,824; 4,941,880; 5,064,413; 5,312,335; 5,312,335; 5,383,851; and 5,399,163.

The compositions of the present invention can be administered to the subject at a suitable dose which can be determined e.g. in dose escalating studies. As set forth above, the construct of the invention exhibiting cross-species specificity as described herein can also be advantageously used in in preclinical testing in non-chimpanzee primates. The dosage regimen will be determined by the attending physician and clinical factors. As is well known in the medical art, dosages for any one patient depend upon many factors, including the patient's size, body surface area, age, the specific compound to be administered, sex, time and route of administration, general health, and other drugs being administered concurrently.

An “effective dose” is an amount of a therapeutic agent that is sufficient to achieve or at least partially achieve a desired effect. A “therapeutically effective dose” is an amount that is sufficient to cure or at least partially arrest the disease and its complications, signs and symptoms in a patient suffering from the disease. Amounts or doses effective for this use will depend on the disease to be treated (the indication), the delivered construct, the therapeutic context and objectives, the severity of the disease, prior therapy, the patient's clinical history and response to the therapeutic agent, the route of administration, the size (body weight, body surface) and/or condition (the age and general health) of the patient, and the general state of the patient's own immune system. The proper dose can be adjusted according to the judgment of the attending physician, to obtain the optimal therapeutic effect.

A therapeutically effective amount of a construct of the invention preferably results in a decrease in severity of disease symptoms, an increase in frequency or duration of disease symptom-free periods or a prevention of impairment or disability due to the disease. In the treatment of tumor antigen-expressing tumors, a therapeutically effective amount of the construct of the invention comprising a binding domain against said tumor antigen preferably inhibits tumor cell growth by at least about 20%, at least about 40%, at least about 50%, at least about 60%, at least about 70%, at least about 80%, or at least about 90% relative to untreated patients. The ability of a compound to inhibit tumor growth may also be evaluated in an animal model predictive of efficacy in human tumors.

In a further embodiment, the invention provides a kit comprising a construct of the invention, a construct produced according to the process of the invention, a polynucleotide of the invention, a vector of the invention, and/or a host cell of the invention. In the context of the present invention, the term “kit” means two or more components—one of which corresponding to the construct, the pharmaceutical composition, the polynucleotide, the vector or the host cell of the invention—packaged together in a container, recipient or otherwise. A kit can hence be described as a set of products and/or utensils that are sufficient to achieve a certain goal, which can be marketed as a single unit.

It is envisaged that a further component of the kit of the invention is an agent, preferably an antibody or construct, which binds to a protein of the immune checkpoint pathway (such as PD-1 or CTLA-4) or to a co-stimulatory immune checkpoint receptor (such as 4-1BB). These agents are described in more detail herein above. According to one embodiment, the kit comprises a construct of the invention and an antibody or construct which binds to PD-1. Anti-PD-1 binding proteins useful for this purpose are e.g. described in detail in PCT/US2019/013205. In certain embodiment, the kit allows for the simultaneous and/or sequential administration of the components.

The kit may comprise one or more recipients (such as vials, ampoules, containers, syringes, bottles, bags) of any appropriate shape, size and material (preferably waterproof, e.g. plastic or glass) containing the construct or the pharmaceutical composition of the present invention in an appropriate dosage for administration (see above). The kit may additionally contain directions for use (e.g. in the form of a leaflet or instruction manual), means for administering the construct or the pharmaceutical composition of the present invention such as a syringe, pump, infuser or the like, means for reconstituting the construct of the invention and/or means for diluting the construct of the invention.

The invention also provides kits for a single-dose administration unit. The kit of the invention may also contain a first recipient comprising a dried/lyophilized construct or pharmaceutical composition and a second recipient comprising an aqueous formulation. In certain embodiments of this invention, kits containing single-chambered and multi-chambered pre-filled syringes are provided.

Whenever the term “construct” is used herein, said term refers to the used polypeptide/polypeptide constructs of the invention or controls thereof as indicated.

As used herein, the singular forms “a”, “an”, and “the” include plural references unless the context clearly indicates otherwise. Thus, for example, reference to “a reagent” includes one or more of such different reagents and reference to “the method” includes reference to equivalent steps and methods known to those of ordinary skill in the art that could be modified or substituted for the methods described herein.

Unless otherwise indicated, the term “at least” preceding a series of elements is to be understood to refer to every element in the series. Those skilled in the art will recognize or be able to ascertain using no more than routine experimentation, many equivalents to the specific embodiments of the invention described herein. Such equivalents are intended to be encompassed by the present invention.

The term “and/or” wherever used herein includes the meaning of “and”, “or” and “all or any other combination of the elements connected by said term”.

The term “about” or “approximately” as used herein means within ±20%, preferably within +15%, more preferably within +10%, and most preferably within ±5% of a given value or range. It also includes the concrete value, e.g., “about 50” includes the value “50”.

Throughout this specification and the claims, unless the context requires otherwise, the word “comprise”, and variations such as “comprises” and “comprising”, will be understood to imply the inclusion of a stated integer or step or group of integers or steps but not the exclusion of any other integer or step or group of integer or step. When used herein the term “comprising” can be substituted with the term “containing” or “including” or sometimes when used herein with the term “having”.

When used herein “consisting of” excludes any element, step, or ingredient not specified in the claim element. When used herein, “consisting essentially of” does not exclude materials or steps that do not materially affect the basic and novel characteristics of the claim.

In each instance herein, any of the terms “comprising”, “consisting essentially of” and “consisting of” may be replaced with either of the other two terms.

The above description and the below examples provide exemplary arrangements, but the present invention is not limited to the specific methodologies, techniques, protocols, material, reagents, substances, etc., described herein and as such can vary. The terminology used herein serves to describe specific embodiments only. The terminology used herein does not intend to limit the scope of the present invention, which is defined solely by the claims. Aspects of the invention are provided in the independent claims. Some optional features of the invention are provided in the dependent claims.

All publications and patents cited throughout the text of this specification (including all patents, patent applications, scientific publications, manufacturer's specifications, instructions, etc.), whether supra or infra, are hereby incorporated by reference in their entirety. Nothing herein is to be construed as an admission that the invention is not entitled to antedate such disclosure by virtue of prior invention. To the extent the material incorporated by reference contradicts or is inconsistent with this specification, the specification will supersede any such material.

A better understanding of the present invention and of its advantages will be obtained from the following examples, offered for illustrative purposes only. The examples are not intended and should not be construed as to limit the scope of the present invention in any way.

### BRIEF DESCRIPTION OF THE DRAWINGS

FIG. 1 A): Effector cells: human unstimulated T cells; Target cells: CHO hu CD70 pEFDHR

Legend:

I2C: CD70 1-A9D CC×I2C×scFc

I2CA: CD70 1-A9D CC×I2C H109A×scFc

8A8.04: CD70 1-A9D CC×8A8.04×scFc

5C3.04: CD70 1-A9D CC×5C3.04×scFc

I2E: CD70 1-A9D CC×5C3.01×scFc

I2K: CD70 1-A9D CC×5G6.05×scFc

5G6.04: CD70 1-A9D CC×5G6.04×scFc

5G6.03: CD70 1-A9D CC×5G6.03×scFc

1D5.04: CD70 1-A9D CC×1D5.04×scFc

8A8.02: CD70 1-A9D CC×8A8.02×scFc

1D5.03: CD70 1-A9D CC×1D5.03×scFc

FIG. 1 B): Effector cells: human unstimulated T cells; Target cells: hu orl FLT3 v1 co CHO #12

Legend:

I2C: FL 7-H3 CC×I2C×scFc

5G6.04: FL 7-H3 CC×5G6.04×scFc

8A8.04: FL 7-H3 CC×8A8.04×scFc

5C3.04: FL 7-H3 CC×5C3.04×scFc

I2E: FL 7-H3 CC×5C3.01×scFc

I2K: FL 7-H3 CC×5G6.05×scFc

I2C CC 44/100: FL 7-H3 CC×I2C CC 44/100×scFc

### EXAMPLES

### Example 1: Thermal Stability

Purified monomeric BiTE® protein at 1 mg/ml formulated in screening buffer was analysed. Thermal stability was determined by Dynamic Light Scattering DLS and Differential Scanning Fluorometry DSF.

DLS

100 μl protein solution were transferred in doubles in a multi well plate, overlaid with paraffin oil and placed in a DLS plate reader (DynaPro Plate Reader II with temperature control, Wyatt Technology Europe GmbH, Dernbach—Germany). Thermal stability was determined in a linear increasing temperature scan from 40° C. to 70° C. while measuring the hydrodynamic radius at the different temperatures in this range. Aggregation temperature was defined at the temperature the radius begins to expand. The mean of both determined aggregation temperatures was calculated and used for candidate ranking.

DSF

Three glass capillaries were filled each with approx. 40 μl protein solution and placed simultaneously into a DSF reader with temperature control (Prometheus nanoDSF, NanoTemper Technologies GmbH, Munich—Germany). Protein internal fluorescence was measured at an excitation wavelength of 280 nm and emission wavelengths 330 and 350 nm in a linear increasing temperature scan from 20° C. to 95° C. Changes in the ratio of the two emission wavelength intensities indicate conformational changes in the protein therefore showing the melting temperature(s) of the analyzed BiTE proteins. The first maximum of the first derivate of the ratio 350/330 nm curve was defined as first melting temperature. The mean of all three determined first melting temperatures was calculated and used for candidate ranking.

The incorporation of specific amino acid mutations in the sequence of the anti-CD3 binder I2C (VH and VL defined in SEQ ID NO: 1854 and 1855, respectively, and linked with a peptide linker of SEQ ID NO: 2; CD70 target binder VH and VL defined in SEQ ID NOs: 2185 and 2186, respectively, and linked with a peptide linker of SEQ ID NO: 2; CD70 binding domain linked to CD3 binding domain with linker of SEQ ID NO: 6; HLE domain as specified in SEQ ID NO: 18 linked with a G4 linker to the C-terminus of the VL region of the CD3 binder; CD70 binding domain is at the N-terminus, followed by the CD3 binding domain with the HLE domain at the C-terminus of the BiTE® molecule) in the BiTE-HLE context resulted in aggregation temperature values of 57.5° C. to 62.0° C. determined by Dynamic Light Scattering (DLS) and melting temperature values of 62.9° C. to 70.3° C. determined by Differential scanning Fluorometry (DSF). The BiTE-HLE molecules harboring the I2C variants showed an aggregation temperature increase of 2.3° C. to 6.8° C. in comparison to the unmodified I2C. The same effect was observed in the melting temperature measurements of the I2C variants resulting in an increase of 2.1° C. to 9.5° C. as compared to the unmodified I2E.

Also the BiTE-HLE construct containing the anti-CD3 binder variant 12 CC44/100, in which an additional disulfide bond was engineered for stabilizing (Reiter, Y. et al., 1994) by replacing the amino acid residues with Cysteine residues at position 44 in the heavy chain variable region and at position 100 in the light chain variable region (numbering scheme according to Kabat et al., 1991), showed a thermostability increase of 3.2° C. for DLS and 5.3° C. for DSF as compared to the unmodified 120. However, said BiTE-HLE construct exhibited a significantly decreased cytotoxic activity (see further example) resulting in a construct which is therefore not suitable for further development and use as a part of T cell engagers.

TABLE 2
Aggregation and melting temperatures of BiTE-HLE molecules
harboring various CD3 binding domains determined
by Dynamic Light Scattering (DLS) or Differential
scanning Fluorometry (DSF), respectively.

| | Thermostability | | |
|---|---|---|---|
| CD3 binder | DLS | DSF | CD3 binding domain |
| in CD70 BITE- | aggregation | melting | SEQ ID NOs: VH, VL, |
| HLE | [° C.] | [° C.] | linker |
| I2E | 59.4 | 68.7 | 2012, 2013, 2 |
| I2K | 58.8 | 64.6 | 2020, 2021, 2 |
| I2L | 60.4 | 69.3 | 2028, 2029, 2 |
| I2M | 60.3 | 67.3 | 2036, 2037, 2 |
| I2M2 | 58.9 | 66.9 | 2044, 2045, 2 |
| 1D5.01 | 62.0 | 70.3 | 42, 43, 2 |
| 1D5.03 | 61.5 | 70.0 | 50, 51, 2 |
| 1D5.04 | 61.8 | 69.7 | 58, 59, 2 |
| 4G10.04 | 59.4 | 66.0 | 66, 67, 2 |
| 5B1.05 | 58.6 | 67.8 | 74, 75, 2 |
| 5B1.09 | 58.8 | 68.2 | 82, 83, 2 |
| 5C3.04 | 60.6 | 69.2 | 90, 91, 2 |
| 5C3.05 | 60.5 | 69.2 | 98, 99, 2 |
| 5G6.03 | 60.1 | 67.4 | 100, 101, 2 |
| 5G6.04 | 59.3 | 65.5 | 108, 109, 2 |
| 6H10.03 | 60.0 | 67.3 | 116, 117, 2 |
| 8A8.02 | 59.4 | 65.9 | 26, 27, 2 |
| 8A8.04 | 59.3 | 67.0 | 34, 35, 2 |
| I2C CC44/100 | 58.4 | 66.1 | |
| I2C H109A | 57.5 | 62.9 | 2560, 2561, 2 |
| I2C | 55.2 | 60.8 | 1854, 1855, 2 |

The mutation of single or more amino acids in the sequence of the anti-SD3 binder I2C in the BiTE-HLE context resulted in aggregation temperature values of 57.0° C. to 62.4C determined by Dynamic Light Scattering (DLS) and melting temperature values of 62.1° C. to 68.8° C. determined by Differential scanning Fluorometry (DSF). The BiTE-HLE molecules harboring the I2C mutants showed an aggregation temperature increase of 1.0° C. to 7.2° C. in comparison to the unmodified 120. The same effect was observed in the melting temperature measurements of the I2C mutants resulting in an increase of 1.0° C. to 7.7° C. as compared to the unmodified I2C. The CD3 binder as referenced in the below Table specifies the amino acid residue exchanges and their positions in the first column; for each entry it is shown in brackets the positions as used in reference to the base sequence of the VH region sequence as defined in SEQ ID NO: 1854 and the VL region sequence as defined in SEQ ID NO: 1855 which is the authoritative nomenclature in accordance with the invention.

TABLE 3

| | | | CD3 |
|---|---|---|---|
| | | | binding |
| | Thermostability | | domain |
| | DLS | DSF | SEQ ID |
| CD3 binder | aggregation | melting | NOs: VH, |
| in CD70 BITE-HLE | [° C.] | [° C.] | VL, linker |
| I2C_HC_A89V_V107A_LC_L21I_Q46K_L83S_G141S | 61.0 | 68.8 | |
| (I2C_HC_A81V_V99A_LC_L20I_Q40K_L69S_G102S) | | | |
| I2C_HC_M41I_A89V_LC_L21I_Q46K_G141S | 62.4 | 68.6 | 1898, 1899, |
| (I2C_HC_M34I_A81V_LC_L20I_Q40K_G102S) | | | 2 |
| I2C_HC_M41I_LC_L21I_Q46K_G141S | 61.9 | 68.3 | 1904, 1905, |
| (I2C_HC_M34I_LC_L20I_Q40K_G102S) | | | 2 |
| I2C_HC_M41I_Q46E_A89V_LC_L21I_Q46K_G141S | 61.4 | 68.2 | 1906, 1907, |
| (I2C_HC_M34I_Q39E_A81V_LC_L20I_Q40K_G102S) | | | 2 |
| I2C_HC_M41I_A89V_LC_Q46K | 60.5 | 66.6 | 1900, 1901, |
| (I2C_HC_M34I_A81V_LC_Q40K) | | | 2 |
| I2C_HC_L52M_LC_L21I_Q46K_G141S | 58.6 | 65.7 | 1866, 1867, |
| (I2C_HC_L45M_LC_L20I_Q40K_G102S) | | | 2 |
| I2C_HC_M41I_S73A_H109A_N114S | 59.0 | 65.4 | |
| (I2C_HC_M34I_S65A_H101A_N106S) | | | |
| I2C_LC_L21I_Q46K_L83S_G141S | 59.0 | 65.3 | |
| (I2C_LC_L20I_Q40K_L69S_G102S) | | | |
| I2C_HC_M41F_A89V (I2C_HC_M34F_A81V) | 59.3 | 65.0 | 1884, 1885, |
| | | | 2 |
| I2C_HC_L52M_LC_Q46K_G141S | 59.5 | 65.0 | 1870, 1871, |
| (I2C_HC_L45M_LC_Q40K_G102S) | | | 2 |
| I2C_HC_Q46D_LC_L21I_Q46K_G141S | 58.3 | 64.7 | 1918 |
| (I2C_HC_Q39D_LC_L20I_Q40K_G102S) | | | 1919, 2 |
| I2C_HC_M41I_A89V (I2C_HC_M34I_A81V) | 59.1 | 64.3 | 1896, 1897, |
| | | | 2 |
| I2C_HC_Q46E_LC_L21I_Q46K_G141S | 58.9 | 64.3 | 1932, 1933, |
| (I2C_HC_Q39E_LC_L20I_Q40K_G102S) | | | 2 |
| I2C_HC_L52M_LC_Q46K | 58.9 | 64.3 | 1868, 1869. |
| (I2C_HC_L45M_LC_Q40K) | | | 2 |
| I2C_HC_M41I_S73A_A89V_V107A_H109A_N114S | 58.1 | 64.2 | |
| (I2C_HC_M34I_S65A_A81V_V99A_H101A_N106S) | | | |
| I2C_HC_Q46D_LC_L21I_Q46R_G141S | 58.2 | 63.7 | 1928, 1929, |
| (I2C_HC_Q39D_LC_L20I_Q40R_G102S) | | | 2 |
| I2C_HC_M41I (I2C_HC_M34I) | 58.2 | 63.6 | 1892, 1893, |
| | | | 2 |
| I2C_LC_Q46K (I2C_LC_Q40K) | 58.4 | 63.5 | 1974, 1975, |
| | | | 2 |
| I2C_HC_Q46D_LC_Q46K_G141S | 58.2 | 63.5 | 1922, 1923, |
| (I2C_HC_Q39D_LC_Q40K_G102S) | | | 2 |
| I2C_LC_L21I_G141S (I2C_LC_L20I_G102S) | 57.4 | 63.2 | |
| I2C_HC_Q46K (I2C_HC_Q39K) | 57.4 | 63.2 | 1944, 1945, |
| | | | 2 |
| I2C_HC_Q46E_LC_Q46K_G141S | 58.0 | 63.2 | 1938, 1939, |
| (I2C_HC_Q39E_LC_Q40K_G102S) | | | 2 |
| I2C_HC_Q46E_LC_L21I_Q46R_G141S | 57.9 | 63.0 | 1934, 1935, |
| (I2C_HC_Q39E_LC_L20I_Q40R_G102S) | | | 2 |
| I2C_HC_Q46D_LC_Q46R_G141S | 57.3 | 62.9 | 1926, 1927, |
| (I2C_HC_Q39D_LC_Q40R_G102S) | | | 2 |
| I2C_HC_A89V (I2C_HC_A81V) | 58.2 | 62.9 | 1862, 1863, |
| | | | 2 |
| I2C_HC_Q46D_LC_Q46K | 57.1 | 62.8 | 1920, 1921, |
| (I2C_HC_Q39D_LC_Q40K) | | | 2 |
| I2C_HC_W136F (I2C_HC_W112F) | 57.0 | 62.7 | 1958, 1959, |
| | | | 2 |
| I2C_HC_W136F_LC_V44I | 57.8 | 62.4 | 1960, 1961, |
| (I2C_HC_W112F_LC_V38I) | | | 2 |
| I2C_HC_Q46E_LC_Q46K | 58.2 | 62.3 | 1936, 1937, |
| (I2C_HC_Q39E_LC_Q40K) | | | 2 |
| I2C_HC_M41V (I2C_HC_M34V) | 57.6 | 62.1 | 1914, 1915, |
| | | | 2 |
| I2C | 55.2 | 60.8 | |

Aggregation and melting temperatures of BiTE-HLE molecules harboring the CD3 binding domain “I2C” (SEQ ID NO: 1854 and 1855, respectively, linked by peptide linker (G₄S)3 (SEQ ID NO: 2)), respectively) with several amino acid mutations determined by Dynamic Light Scattering (DLS) or Differential scanning Fluorometry (DSF), respectively, as described herein above.

### Example 2: Cytotoxic Activity

Cytotoxic Activity

Human peripheral blood mononuclear cells (PBMC) were prepared by Ficoll density gradient centrifugation from enriched lymphocyte preparations (buffy coats), a side product of blood banks collecting blood for transfusions. Buffy coats were supplied by a local blood bank and PBMC were prepared on the same day of blood collection. After Ficoll density centrifugation and extensive washes with Dulbecco's PBS (Gibco), remaining erythrocytes were removed from PBMC via incubation with erythrocyte lysis buffer (155 mM NH₄Cl, 10 mM KHCO₃, 100 μM EDTA). Platelets were removed via the supernatant upon centrifugation of PBMC at 100×g. Remaining lymphocytes mainly encompass B and T lymphocytes, NK cells and monocytes. PBMC were kept in culture at 37° C./5% CO₂ in RPMI medium (Gibco) with 10% FCS (Gibco).

Depletion of CD14⁺ and CD56⁺ Cells

For depletion of CD14⁺ cells, human CD14 MicroBeads (Milteny Biotec, MACS, #130-050-201) were used, for depletion of NK cells human CD56 MicroBeads (MACS, #130-050-401). PBMC were counted and centrifuged for 10 min at room temperature with 300×g. The supernatant was discarded and the cell pellet resuspended in MACS isolation buffer [80 μL/10⁷ cells; PBS (Invitrogen, #20012-043), 0.5% (v/v) FBS (Gibco, #10270-106), 2 mM EDTA (Sigma-Aldrich, #E-6511)]. CD14 MicroBeads and CD56 MicroBeads (20 μL/10⁷ cells) were added and incubated for 15 min at 4-8° C. The cells were washed with MACS isolation buffer (1-2 mL/10⁷ cells). After centrifugation (see above), supernatant was discarded and cells resuspended in MACS isolation buffer (500 μL/10⁸ cells). CD14/CD56 negative cells were then isolated using LS Columns (Miltenyi Biotec, #130-042-401). PBMC w/o CD14+/CD56+ cells were cultured in RPMI complete medium i.e. RPMI1640 (Biochrom AG, #FG1215) supplemented with 10% FBS (Biochrom AG, #S0115), 1×non-essential amino acids (Biochrom AG, #K0293), 10 mM Hepes buffer (Biochrom AG, #L1613), 1 mM sodium pyruvate (Biochrom AG, #L0473) and 100 U/mL penicillin/streptomycin (Biochrom AG, #A2213) at 37° C. in an incubator until needed.

Target Cell Labeling

For the analysis of cell lysis in flow cytometry assays, the fluorescent membrane dye DiOC₁₈ (DiO) (Molecular Probes, #V22886) was used to label human target-transfected CHO cells as target cells and distinguish them from effector cells. Briefly, cells were harvested, washed once with PBS and adjusted to 10⁶ cell/mL in PBS containing 2% (v/v) FBS and the membrane dye DiO (5 μL/10⁶ cells). After incubation for 3 min at 37° C., cells were washed twice in complete RPMI medium and the cell number adjusted to 1.25×10⁵ cells/mL. The vitality of cells was determined using the NC-250 cell counter (Chemometec).

Flow Cytometry Based Analysis

This assay was designed to quantify the lysis of human target-transfected CHO cells in the presence of serial dilutions of anti-target multi-specific constructs. Equal volumes of DiO-labeled target cells and effector cells (i.e., PBMC w/o CD14⁺ and CD56⁺ cells) were mixed, resulting in an E:T cell ratio of 10:1. 80 μl of this suspension were transferred to each well of a 96-well plate. 20 μL of serial dilutions of the anti-target×anti-CD3 multi-specific constructs and a negative control (a CD3-based multi-specific construct recognizing an irrelevant target antigen) or RPMI complete medium as an additional negative control were added. The multi-specific construct-mediated cytotoxic reaction proceeded for 48 hours in a 7% CO₂ humidified incubator. Then cells were transferred to a new 96-well plate and loss of target cell membrane integrity was monitored by adding propidium iodide (PI) at a final concentration of 1 pg/mL. PI is a membrane impermeable dye that normally is excluded from viable cells, whereas dead cells take it up and become identifiable by fluorescent emission.

Samples were measured by flow cytometry on an iQue Plus instrument and analyzed by Forecyt software (both from Intellicyt). Target cells were identified as DiO-positive cells. PI-negative target cells were classified as living target cells. Percentage of cytotoxicity was calculated according to the following formula:

$$Cytotoxicity [ \% ] = \frac{{n}_{dead target cells}}{{n}_{target cells}} \times 100$$

$$n = number of events$$

Using GraphPad Prism 6 software (Graph Pad Software, San Diego), the percentage of cytotoxicity was plotted against the corresponding bispecific construct concentrations. Dose response curves were analyzed with the four parametric logistic regression models for evaluation of sigmoid dose response curves with fixed hill slope and EC50 values were calculated.

Results:

TABLE 4
EC50 values in pM of T-cell engager with different anti-CD3 scFvs

| | EC50 [pM] |
|---|---|
| I2C | 0.2 |
| I2CA | 1.2 |
| 8A8.04 | 0.1 |
| 5C3.04 | 0.1 |
| I2E | 0.05 |
| I2K | 0.1 |
| 5G6.04 | 0.1 |
| 5G6.03 | 0.1 |
| 1D5.04 | 0.4 |
| 8A8.02 | 0.1 |
| 1D5.03 | 0.2 |

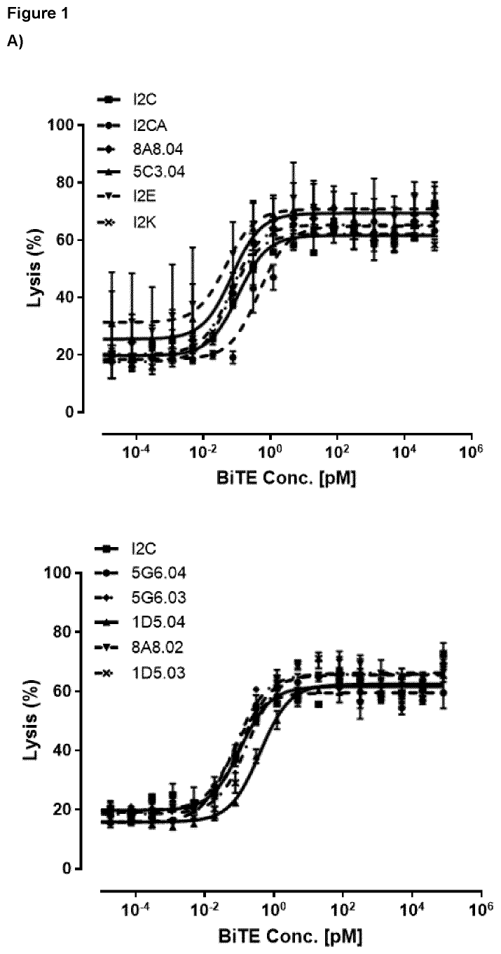

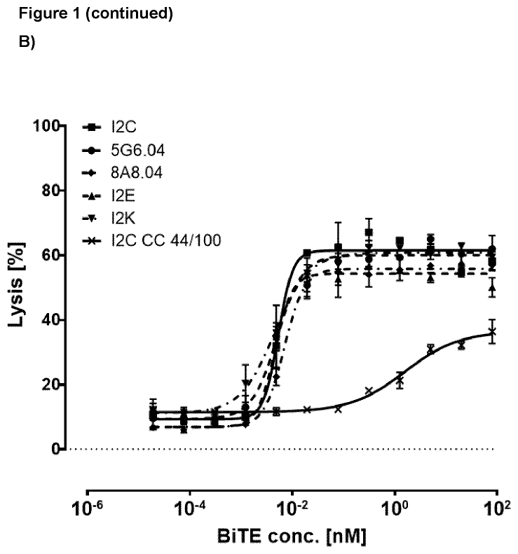

T-cell engager with anti-CD3 scFvs I2C, I2CA, 8A8.04, 5C3.04, 12E, I2K, 5G6.04, 5G6.03, 1D5.04, 8A8.02 and 1D5.03 show similar EC50 values in range from 0.05 pM to 0.4 pM (FIG. 1 A) and Table 4).

TABLE 5
EC50 values in pM of T-cell engager with different anti-CD3 scFvs

| | EC50 [pM] |
|---|---|
| I2C | 5.3 |
| 5G6.04 | 5.1 |
| 8A8.04 | 7.0 |
| I2E | 4.5 |
| I2K | 4.5 |
| I2C CC 44/100 | 1578 |

T-cell engager with anti-CD3 scFvs I2C, 5G6.04, 8A8.04, 12E and I2K show similar EC50 values in the range from 4.5 pM to 7.0 pM. T-cell engager with anti-CD3 scFv I2C CC 44/100 shows an EC50 value of 1578 pM and is 297-fold less active compared to T-cell engager with anti-CD3 scFv I2C (FIG. 1 B) and Table 5).

### Sequences

The below sequence list is the authoritative list for SEQ ID NOs referred to in the specification text and claims and may contain (a) gap(s) as to consecutive numbering. Any further list, such as the list complying with WIPO Standard ST.25 that contains consecutive numberings has been generated of this list and the difference in Sequence numbers is to be reconciled with the below list. Sequence identity can be identified without further ado by reference to the unique entry in the second column identified as “designation” as well as a sequence alignment:

| SEQ | | | |
|---|---|---|---|
| ID | | | |
| NO: | Designation | Organism | Sequence |
| 1 | (G4S)10- | artificial | GGGGGGGGSGGGGSGGGGSGGGGSGGGGSGGGGSGGGGSGGGGSGGGGS |
| | Linker | | |
| 2 | (G4S)3- | artificial | GGGGSGGGGSGGGGS |
| | Linker | | |
| 3 | G(EAAAK)10- | artificial | GEAAAKEAAAKEAAAKEAAAKEAAAKEAAAKEAAAKEAAAKEAAAKEAAAK |
| | Linker | | |
| 4 | G4-Linker | artificial | GGGG |
| 5 | G4Q- | artificial | GGGGQ |
| | Linker | | |
| 6 | G4S- | artificial | GGGGS |
| | Linker | | |
| 7 | S(G4S)10 | artificial | SGGGGSGGGGSGGGGSGGGGSGGGGSGGGGSGGGGSGGGGSGGGGSGGGGS |
| | Linker | | |
| 8 | S(G4S)3- | artificial | SGGGGSGGGGSGGGGS |
| | Linker | | |
| 9 | SG(EAAAK)10- | artificial | SGEAAAKEAAAKEAAAKEAAAKEAAAKEAAAKEAAAKEAAAKEAAAKEAAAK |
| | Linker | | |
| 10 | SG4Q- | artificial | SGGGGQ |
| | Linker | | |
| 11 | SG4S- | artificial | SGGGGS |
| | Linker | | |
| 12 | (EAAAK)10- | artificial | EAAAKEAAAKEAAAKEAAAKEAAAKEAAAKEAAAKEAAAKEAAAKEAAAK |
| | Spacer | | |
| 13 | (G4S)10- | artificial | GGGGSGGGGSGGGGSGGGGSGGGGSGGGGSGGGGSGGGGSGGGGSGGGGS |
| | Spacer | | |
| 14 | 2x scFc- | artificial | DKTHTCPPCPAPELLGGPSVFLFPPKPKDTLMISRTPEVTCVVVDVSHEDPEVKENWY |
| | Spacer | | VDGVEVHNAKTKPCEEQYGSTYRCVSVLTVLHQDWLNGKEYKCKVSNKALPAPIEKTI |
| | | | SKAKGQPREPQVYTLPPSREEMTKNQVSLTCLVKGFYPSDIAVEWESNGQPENNYKTT |
| | | | PPVLDSDGSFFLYSKLTVDKSRWQQGNVFSCSVMHEALHNHYTQKSLSLSPGKGGGGS |
| | | | GGGGSGGGGSGGGGSGGGGSGGGGSDKTHTCPPCPAPELLGGPSVFLFPPKPKDTLMI |
| | | | SRTPEVTCVVVDVSHEDPEVKFNWYVDGVEVHNAKTKPCEEQYGSTYRCVSVLTVLHQ |
| | | | DWLNGKEYKCKVSNKALPAPIEKTISKAKGQPREPQVYTLPPSREEMTKNQVSLTCLV |
| | | | KGFYPSDIAVEWESNGQPENNYKTTPPVLDSDGSFFLYSKLTVDKSRWQQGNVFSCSV |
| | | | MHEALHNHYTQKSLSLSPGKDKTHTCPPCPAPELLGGPSVFLFPPKPKDTLMISRTPE |
| | | | VTCVVVDVSHEDPEVKFNWYVDGVEVHNAKTKPCEEQYGSTYRCVSVLTVLHQDWLNG |
| | | | KEYKCKVSNKALPAPIEKTISKAKGQPREPQVYTLPPSREEMTKNQVSLTCLVKGFYP |
| | | | SDIAVEWESNGQPENNYKTTPPVLDSDGSFFLYSKLTVDKSRWQQGNVFSCSVMHEAL |
| | | | HNHYTQKSLSLSPGKGGGGSGGGGSGGGGSGGGGSGGGGSGGGGSDKTHTCPPCPAPE |
| | | | LLGGPSVFLFPPKPKDTLMISRTPEVTCVVVDVSHEDPEVKENWYVDGVEVHNAKTKP |
| | | | CEEQYGSTYRCVSVLTVLHQDWLNGKEYKCKVSNKALPAPIEKTISKAKGQPREPQVY |
| | | | TLPPSREEMTKNQVSLTCLVKGFYPSDIAVEWESNGQPENNYKTTPPVLDSDGSFFLY |
| | | | SKLTVDKSRWQQGNVFSCSVMHEALHNHYTQKSLSLSPGK |
| 15 | G4S- | artificial | GGGGS |
| | Spacer | | |
| 16 | Human Serum | artificial | DAHKSEVAHRFKDLGEENFKALVLIAFAQYLQQCPFEDHVKLVNEVTEFAKTCVADES |
| | Albumin | | AENCDKSLHTLFGDKLCTVATLRETYGEMADCCAKQEPERNECFLQHKDDNPNLPRLV |
| | (HSA)- | | RPEVDVMCTAFHDNEETFLKKYLYEIARRHPYFYAPELLFFAKRYKAAFTECCQAADK |
| | Spacer | | AACLLPKLDELRDEGKASSAKQRLKCASLQKFGERAFKAWAVARLSQRFPKAEFAEVS |
| | | | KLVTDLTKVHTECCHGDLLECADDRADLAKYICENQDSISSKLKECCEKPLLEKSHCI |
| | | | AEVENDEMPADLPSLAADFVESKDVCKNYAEAKDVFLGMFLYEYARRHPDYSVVLLLR |
| | | | LAKTYETTLEKCCAAADPHECYAKVEDEFKPLVEEPQNLIKQNCELFEQLGEYKFQNA |
| | | | LLVRYTKKVPQVSTPTLVEVSRNLGKVGSKCCKHPEAKRMPCAEDYLSVVLNQLCVLH |
| | | | EKTPVSDRVTKCCTESLVNRRPCFSALEVDETYVPKEFNAETFTFHADICTLSEKERQ |
| | | | IKKQTALVELVKHKPKATKEQLKAVMDDFAAFVEKCCKADDKETCFAEEGKKLVAASQ |
| | | | AALGL |
| 17 | PD1 (ECM | artificial | LDSPDRPWNPPTFSPALLVVTEGDNATFTCSFSNTSESFVLNWYRMSPSNQTDKLAAF |
| | 25-167) | | PEDRSQPGQDCRFRVTQLPNGRDFHMSVVRARRNDSGTYLCGAISLAPKAQIKESLRA |
| | Spacer | | ELRVTERRAEVPTAHPSPSPRPAGQFQ |
| 18 | ScFc- | artificial | DKTHTCPPCPAPELLGGPSVFLFPPKPKDTLMISRTPEVTCVVVDVSHEDPEVKENWY |
| | Spacer | | VDGVEVHNAKTKPCEEQYGSTYRCVSVLTVLHQDWLNGKEYKCKVSNKALPAPIEKTI |
| | | | SKAKGQPREPQVYTLPPSREEMTKNQVSLTCLVKGFYPSDIAVEWESNGQPENNYKTT |
| | | | PPVLDSDGSFFLYSKLTVDKSRWQQGNVFSCSVMHEALHNHYTQKSLSLSPGKGGGGS |
| | | | GGGGSGGGGSGGGGSGGGGSGGGGSDKTHTCPPCPAPELLGGPSVFLFPPKPKDTLMI |
| | | | SRTPEVTCVVVDVSHEDPEVKFNWYVDGVEVHNAKTKPCEEQYGSTYRCVSVLTVLHQ |
| | | | DWLNGKEYKCKVSNKALPAPIEKTISKAKGQPREPQVYTLPPSREEMTKNQVSLTCLV |
| | | | KGFYPSDIAVEWESNGQPENNYKTTPPVLDSDGSFFLYSKLTVDKSRWQQGNVFSCSV |
| | | | MHEALHNHYTQKSLSLSPGK |
| 19 | scFc_mod GQ- | artificial | CPPCPAPELLGGPSVFLFPPKPKDTLMISRTPEVTCVVVDVSHEEPEVKENWYVDGVE |
| | Spacer | | VHNAKTKPCEEQYGSTYRCVSVLTVLHQDWLNGKEYKCKVSNKALPAPIEKTISKAKG |
| | | | QPREPQVYTLPPSREEMTKNQVSLTCLVKGFYPSDIAVEWESNGQPENNYKTTPPVLD |
| | | | SDGSFFLYSKLTVDKSRWQQGNVFSCSVMHEALHNHYTQKSLSLSPGKGGGGQGGGGQ |
| | | | GGGGQGGGGQGGGGQGGGGQCPPCPAPELLGGPSVFLFPPKPKDTLMISRTPEVTCVV |
| | | | VDVSHEEPEVKFNWYVDGVEVHNAKTKPCEEQYGSTYRCVSVLTVLHQDWLNGKEYKC |
| | | | KVSNKALPAPIEKTISKAKGQPREPQVYTLPPSREEMTKNQVSLTCLVKGFYPSDIAV |
| | | | EWESNGQPENNYKTTPPVLDSDGSFFLYSKLTVDKSRWQQGNVFSCSVMHEALHNHYT |
| | | | QKSLSLSPGK |
| 20 | 16278-A8.2- | artificial | KYAIN |
| | HCDR1 | | |
| 21 | 16278-A8.2- | artificial | RIRSKYNNYATYYADAVKD |
| | HCDR2 | | |
| 22 | 16278-A8.2- | artificial | AGNFGTSYISYWAY |
| | HCDR3 | | |
| 23 | 16278-A8.2- | artificial | GSSTGAVTSGNYPN |
| | LCDR1 | | |
| 24 | 16278-A8.2- | artificial | GTKFLAP |
| | LCDR2 | | |
| 25 | 16278-A8.2- | artificial | ALWYSNRWV |
| | LCDR3 | | |
| 26 | 16278-A8.2- | artificial | EVQLVESGGGLVQPGGSLKLSCAASGFTFNKYAINWVRQAPGKGLEWVARIRSKYNNY |
| | VH | | ATYYADAVKDRFTISRDDSKNTAYLQMNNLKTEDTAVYYCARAGNFGTSYISYWAYWG |
| | | | QGTLVTVSS |
| 27 | 16278-A8.2- | artificial | QTVVTQEPSLTVSPGGTVTLTCGSSTGAVTSGNYPNWVQKKPGQAPRGLIGGTKFLAP |
| | L | | GTPARFSGSLLGGKAALTLSGVQPEDEAEYYCALWYSNRWVFGSGTKLTVL |
| 28 | 16278-A8.4- | artificial | KYAIN |
| | HCDR1 | | |
| 29 | 16278-A8.4- | artificial | RIRSKYNNYATYYAEAVKD |
| | HCDR2 | | |
| 30 | 16278-A8.4- | artificial | AGNFGTSYISYWAY |
| | HCDR3 | | |
| 31 | 16278-A8.4 | artificial | GSSTGAVTSGNYPN |
| | -LCDR1 | | |
| 32 | 16278-A8.4 | artificial | GTKFLAP |
| | -LCDR2 | | |
| 33 | 16278-A8.4 | artificial | ALWYSNRWV |
| | -LCDR3 | | |
| 34 | 16278-A8.4 | artificial | EVQLVESGGGLVQPGGSLKLSCAASGFTFNKYAINWVRQAPGKGLEWVARIRSKYNNY |
| | -VH | | ATYYAEAVKDRFTISRDDSKNTAYLQMNNLKTEDTAVYYCARAGNFGTSYISYWAYWG |
| | | | QGTLVTVSS |
| 35 | 16278-A8.4 | artificial | QTVVTQEPSLTVSPGGTVTLTCGSSTGAVTSGNYPNWVQKKPGQAPRGLIGGTKFLAP |
| | -VL | | GTPARFSGSLLGGKAALTLSGVQPEDEAEYYCALWYSNRWVFGSGTKLTVL |
| 36 | 16281-D5.1 | artificial | KYAIN |
| | -HCDR1 | | |
| 37 | 16281-D5.1 | artificial | RIRSKYNNYATYYADAVKD |
| | -HCDR2 | | |
| 38 | 16281-D5.1 | artificial | AGNFGSSYISYFAY |
| | -HCDR3 | | |
| 39 | 16281-D5.1 | artificial | GSSTGAVTSGNYPN |
| | -LCDR1 | | |
| 40 | 16281-D5.1 | artificial | GTKFLAP |
| | -LCDR2 | | |
| 41 | 16281-D5.1 | artificial | ALWYSNRWV |
| | -LCDR3 | | |
| 42 | 16281-D5.1 | artificial | EVQLVESGGGLVQPGGSLKLSCAASGFTFNKYAINWVRQAPGKGLEWVARIRSKYNNY |
| | -VH | | ATYYADAVKDRFTISRDDSKNTAYLQMNNLKTEDTAVYYCVRAGNFGSSYISYFAYWG |
| | | | QGTLVTVSS |
| 43 | 16281-D5.1 | artificial | QTVVTQEPSLTVSPGGTVTLTCGSSTGAVTSGNYPNWVQKKPGQAPRGLIGGTKFLAP |
| | -VL | | GTPARFSGSLLGGKAALTLSGVQPEDEAEYYCALWYSNRWVFGSGTKLTVL |
| 44 | 16281-D5.3 | artificial | KYAIN |
| | -HCDR1 | | |
| 45 | 16281-D5.3 | artificial | RIRSKYNNYATYYAEAVKD |
| | -HCDR2 | | |
| 46 | 16281-D5.3 | artificial | AGNFGSSYISYFAY |
| | -HCDR3 | | |
| 47 | 16281-D5.3 | artificial | GSSTGAVTSGNYPN |
| | -LCDR1 | | |
| 48 | 16281-D5.3 | artificial | GTKFLAP |
| | -LCDR2 | | |
| 49 | 16281-D5.3 | artificial | ALWYSNRWV |
| | -LCDR3 | | |
| 50 | 16281-D5.3 | artificial | EVQLVESGGGLVQPGGSLKLSCAASGFTFNKYAINWVRQAPGKGLEWVARIRSKYNNY |
| | -VH | | ATYYAEAVKDRFTISRDDSKNTAYLQMNNLKTEDTAVYYCVRAGNFGSSYISYFAYWG |
| | | | QGTLVTVSS |
| 51 | 16281-D5.3 | artificial | QTVVTQEPSLTVSPGGTVTLTCGSSTGAVTSGNYPNWVQKKPGQAPRGLIGGTKFLAP |
| | -VL | | GTPARFSGSLLGGKAALTLSGVQPEDEAEYYCALWYSNRWVFGSGTKLTVL |
| 52 | 16281-D5.4 | artificial | KYAIN |
| | -HCDR1 | | |
| 53 | 16281-D5.4 | artificial | RIRSKYNNYATYYAEAVKD |
| | -HCDR2 | | |
| 54 | 16281-D5.4 | artificial | AGNFGTSYISYFAY |
| | -HCDR3 | | |
| 55 | 16281-D5.4 | artificial | GSSTGAVTSGNYPN |
| | -LCDR1 | | |
| 56 | 16281-D5.4 | artificial | GTKFLAP |
| | -LCDR2 | | |
| 57 | 16281-D5.4 | artificial | ALWYSNRWV |
| | -LCDR3 | | |
| 58 | 16281-D5.4 | artificial | EVQLVESGGGLVQPGGSLKLSCAASGFTFNKYAINWVRQAPGKGLEWVARIRSKYNNY |
| | -VH | | ATYYAEAVKDRFTISRDDSKNTAYLQMNNLKTEDTAVYYCVRAGNFGTSYISYFAYWG |
| | | | QGTLVTVSS |
| 59 | 16281-D5.4 | artificial | QTVVTQEPSLTVSPGGTVTLTCGSSTGAVTSGNYPNWVQKKPGQAPRGLIGGTKFLAP |
| | -VL | | GTPARFSGSLLGGKAALTLSGVQPEDEAEYYCALWYSNRWVFGSGTKLTVL |
| 60 | 16334-G10.4 | artificial | KYAMN |
| | -HCDR1 | | |
| 61 | 16334-G10.4 | artificial | RIRSKYNNYATYYAEAVKD |
| | -HCDR2 | | |
| 62 | 16334-G10.4 | artificial | AENIGTSYISYWAY |
| | -HCDR3 | | |
| 63 | 16334-G10.4 | artificial | GSSTGAVTSGNYPN |
| | -LCDR1 | | |
| 64 | 16334-G10.4 | artificial | GTKFLAP |
| | -LCDR2 | | |
| 65 | 16334-G10.4 | artificial | VLWYSNRWV |
| | -LCDR3 | | |
| 66 | 16334-G10.4 | artificial | EVQLVESGGGLVQPGGSLKLSCAASGFTFSKYAMNWVREAPGKGLEWVARIRSKYNNY |
| | -VH | | ATYYAEAVKDRFTISRDDSKNTVYLQMNNLKTEDTAVYYCVRAENIGTSYISYWAYWG |
| | | | QGTLVTVSS |
| 67 | 16334-G10.4 | artificial | QTVVTQEPSLTVSPGGTVTMTCGSSTGAVTSGNYPNWVQKKPGQAPRGLIGGTKFLAP |
| | -VL | | GTPARFSGSLEGGKAALTLSGVQPEDEAEYYCVLWYSNRWVFGGGTKLTVL |
| 68 | 16335-B1.5 | artificial | KYAMN |
| | -HCDR1 | | |
| 69 | 16335-B1.5 | artificial | RIRSKYNNYATYYAEAVKG |
| | -HCDR2 | | |
| 70 | 16335-B1.5 | artificial | AGNFGSSYISYWAY |
| | -HCDR3 | | |
| 71 | 16335-B1.5 | artificial | GSSTGAVTSGNYPN |
| | -LCDR1 | | |
| 72 | 16335-B1.5 | artificial | GTKFLAP |
| | -LCDR2 | | |
| 73 | 16335-B1.5 | artificial | VLWYSNRWV |
| | -LCDR3 | | |
| 74 | 16335-B1.5 | artificial | EVQLVESGGGLVQPGGSLKLSCAASGFTFSKYAMNWVRQAPGKGMEWVARIRSKYNNY |
| | -VH | | ATYYAEAVKGRFTISRDDSKNTVYLQMNNLKTEDTAVYYCVRAGNFGSSYISYWAYWG |
| | | | QGTLVTVSS |
| 75 | 16335-B1.5 | artificial | QTVVTQEPSLTVSPGGTVTLTCGSSTGAVTSGNYPNWVQKKPGQAPRGLIGGTKFLAP |
| | -VL | | GTPARFSGSLSGGKAALTLSGVQPEDEAEYYCVLWYSNRWVFGGGTKLTVL |
| 76 | 16335-B1.9 | artificial | KYAMN |
| | -HCDR1 | | |
| 77 | 16335-B1.9 | artificial | RIRSKYNNYATYYADAVKG |
| | -HCDR2 | | |
| 78 | 16335-B1.9 | artificial | AGNFGTSYISYFAY |
| | -HCDR3 | | |
| 79 | 16335-B1.9 | artificial | GSSTGAVTSGNYPN |
| | -LCDR1 | | |
| 80 | 16335-B1.9 | artificial | GTKFLAP |
| | -LCDR2 | | |
| 81 | 16335-B1.9 | artificial | VLYYSNRWV |
| | -LCDR3 | | |
| 82 | 16335-B1.9 | artificial | EVQLVESGGGLVQPGGSLKLSCAASGFTFSKYAMNWVRQAPGKGMEWVARIRSKYNNY |
| | -VH | | ATYYADAVKGRFTISRDDSKNTVYLQMNNLKTEDTAVYYCVRAGNFGTSYISYFAYWG |
| | | | QGTLVTVSS |
| 83 | 16335-B1.9 | artificial | QTVVTQEPSLTVSPGGTVTLTCGSSTGAVTSGNYPNWVQKKPGQAPRGLIGGTKFLAP |
| | -VL | | GTPARFSGSLSGGKAALTLSGVQPEDEAEYYCVLYYSNRWVFGGGTKLTVL |
| 84 | 16335-C3.4 | artificial | KYAIN |
| | -HCDR1 | | |
| 85 | 16335-C3.4 | artificial | RIRSKYNNYATYYADAVKD |
| | -HCDR2 | | |
| 86 | 16335-C3.4 | artificial | AENIGTSYISYWAY |
| | -HCDR3 | | |
| 87 | 16335-C3.4 | artificial | GSSTGAVTSGNYPN |
| | -LCDR1 | | |
| 88 | 16335-C3.4 | artificial | GTKFLAP |
| | -LCDR2 | | |
| 89 | 16335-C3.4 | artificial | VLWYSNRWV |
| | -LCDR3 | | |
| 90 | 16335-C3.4 | artificial | EVQLVESGGGLVQPGGSLKLSCAASGFTFNKYAINWVRQAPGKGLEWVARIRSKYNNY |
| | -VH | | ATYYADAVKDRFTISRDDSKNTVYLQMNNLKTEDTAVYYCARAENIGTSYISYWAYWG |
| | | | QGTLVTVSS |
| 91 | 16335-C3.4 | artificial | QTVVTQEPSLTVSPGGTVTITCGSSTGAVTSGNYPNWVQKKPGQAPRGLIGGTKFLAP |
| | -VL | | GTPARFSGSLSGGKAALTLSGVQPEDEAEYYCVLWYSNRWVFGSGTKLTVL |
| 92 | 16335-C3.5 | artificial | KYAIN |
| | -HCDR1 | | |
| 93 | 16335-C3.5 | artificial | RIRSKYNNYATYYAEAVKD |
| | -HCDR2 | | |
| 94 | 16335-C3.5 | artificial | AGNFGSSYISYWAY |
| | -HCDR3 | | |
| 95 | 16335-C3.5 | artificial | GSSTGAVTSGNYPN |
| | -LCDR1 | | |
| 96 | 16335-C3.5 | artificial | GTKFLAP |
| | -LCDR2 | | |
| 97 | 16335-C3.5 | artificial | VLYYSNRWV |
| | -LCDR3 | | |
| 98 | 16335-C3.5 | artificial | EVQLVESGGGLVQPGGSLKLSCAASGFTENKYAINWVRQAPGKGLEWVARIRSKYNNY |
| | -VH | | ATYYAEAVKDRFTISRDDSKNTVYLQMNNLKTEDTAVYYCARAGNFGSSYISYWAYWG |
| | | | QGTLVTVSS |
| 99 | 16335-C3.5 | artificial | QTVVTQEPSLTVSPGGTVTITCGSSTGAVTSGNYPNWVQKKPGQAPRGLIGGTKFLAP |
| | -VL | | GTPARFSGSLSGGKAALTLSGVQPEDEAEYYCVLYYSNRWVFGSGTKLTVL |
| 100 | 16335-G6.3 | artificial | EVQLVESGGGLVQPGGSLKLSCAASGFTFNKYAMNWVRQAPGKGLEWVARIRSKYNNY |
| | -VH | | ATYYAEAVKGRFTISRDDSKNTVYLQMNNLKTEDTAVYYCVRAGNFGSSYISYWAYWG |
| | | | QGTLVTVSS |
| 101 | 16335-G6.3 | artificial | QTVVTQEPSLTVSPGGTVTITCGSSTGAVTSGNYPNWVQQKPGQAPRGLIGGTKFLAP |
| | -VL | | GTPARFSGSLSGGKAALTLSGVQPEDEAEYYCVLWYSNRWVFGSGTKLTVL |
| 102 | 16335-G6.4 | artificial | KYAMN |
| | -HCDR1 | | |
| 103 | 16335-G6.4 | artificial | RIRSKYNNYATYYADAVKG |
| | -HCDR2 | | |
| 104 | 16335-G6.4 | artificial | NENIGSSYISYWAY |
| | -HCDR3 | | |
| 105 | 16335-G6.4 | artificial | GSSTGAVTSGNYPN |
| | -LCDR1 | | |
| 106 | 16335-G6.4 | artificial | GTKFLAP |
| | -LCDR2 | | |
| 107 | 16335-G6.4 | artificial | VLWYSNRWV |
| | -LCDR3 | | |
| 108 | 16335-G6.4 | artificial | EVQLVESGGGLVQPGGSLKLSCAASGFTFNKYAMNWVRQAPGKGLEWVARIRSKYNNY |
| | -VH | | ATYYADAVKGRFTISRDDSKNTVYLQMNNLKTEDTAVYYCVRNENIGSSYISYWAYWG |
| | | | QGTLVTVSS |
| 109 | 16335-G6.4 | artificial | QTVVTQEPSLTVSPGGTVTITCGSSTGAVTSGNYPNWVQQKPGQAPRGLIGGTKFLAP |
| | -VL | | GTPARFSGSLSGGKAALTLSGVQPEDEAEYYCVLWYSNRWVFGSGTKLTVL |
| 110 | 16356-H10.3 | artificial | KYAMN |
| | -HCDR1 | | |
| 111 | 16356-H10.3 | artificial | RIRSKYNNYATYYAEAVKD |
| | -HCDR2 | | |
| 112 | 16356-H10.3 | artificial | AGNFGTSYISYWAY |
| | -HCDR3 | | |
| 113 | 16356-H10.3 | artificial | GSSTGAVTSGNYPN |
| | -LCDR1 | | |
| 114 | 16356-H10.3 | artificial | GTKFLAP |
| | -LCDR2 | | |
| 115 | 16356-H10.3 | artificial | VLWYSNRWV |
| | -LCDR3 | | |
| 116 | 16356-H10.3 | artificial | EVQLVESGGGLVQPGGSLKLSCAASGFTFNKYAMNWVRQAPGKGMEWVARIRSKYNNY |
| | -VH | | ATYYAEAVKDRFTISRDDSKNTLYLQMNNLKTEDTAVYYCVRAGNFGTSYISYWAYWG |
| | | | QGTLVTVSS |
| 117 | 16356-H10.3 | artificial | QTVVTQEPSLTVSPGGTVTITCGSSTGAVTSGNYPNWIQKKPGQAPRGLIGGTKFLAP |
| | -VL | | GTPARFSGSLEGGKAALTLSGVQPEDEAEYYCVLWYSNRWVFGSGTKLTVL |
| 118 | 2B2_Comp1- | artificial | KYAIN |
| | HCDR1 | | |
| 119 | 2B2_Comp1- | artificial | RIRSKYNNYATYYADQVKD |
| | HCDR2 | | |
| 120 | 2B2_Comp1- | artificial | HANFGNSYISYWAY |
| | HCDR3 | | |
| 121 | 2B2_Comp1- | artificial | ASSTGAVTSGNYPN |
| | LCDR1 | | |
| 122 | 2B2_Comp1- | artificial | GTKFLVP |
| | LCDR2 | | |
| 123 | 2B2_Comp1- | artificial | TLWYSNRWV |
| | LCDR3 | | |
| 124 | 2B2_Comp1- | artificial | EVQLVESGGGLVQPGGSLKLSCAASGFTFNKYAINWVRQAPGKGLEWVARIRSKYNNY |
| | VH | | ATYYADQVKDRFTISRDDSKNTAYLQMNNLKTEDTAVYYCVRHANFGNSYISYWAYWG |
| | | | QGTLVTVSS |
| 125 | 2B2_Comp1- | artificial | QTVVTQEPSLTVSPGGTVTLTCASSTGAVTSGNYPNWVQQKPGQAPRGLIGGTKFLVP |
| | VL | | GTPARFSGSLLGGKAALTLSGVQPEDEAEYYCTLWYSNRWVFGGGTKLTVL |
| 126 | 2B2_Comp1_ | artificial | KYAIN |
| | I2E-HCDR1 | | |
| 127 | 2B2_Comp1_ | artificial | RIRSKYNNYATYYADAVKD |
| | I2E-HCDR2 | | |
| 128 | 2B2_Comp1_ | artificial | AANFGSSYISYWAY |
| | I2E-HCDR3 | | |
| 129 | 2B2_Comp1_ | artificial | ASSTGAVTSGNYPN |
| | I2E-LCDR1 | | |
| 130 | 2B2_Comp1_ | artificial | GTKFLVP |
| | I2E-LCDR2 | | |
| 131 | 2B2_Comp1_ | artificial | TLWYSNRWV |
| | I2E-LCDR3 | | |
| 132 | 2B2_Comp1_ | artificial | EVQLVESGGGLVQPGGSLKLSCAASGFTFNKYAINWVRQAPGKGLEWVARIRSKYNNY |
| | I2E-VH | | ATYYADAVKDRFTISRDDSKNTVYLQMNNLKTEDTAVYYCARAANFGSSYISYWAYWG |
| | | | QGTLVTVSS |
| 133 | 2B2_Comp1_ | artificial | QTVVTQEPSLTVSPGGTVTITCASSTGAVTSGNYPNWVQKKPGQAPRGLIGGTKFLVP |
| | I2E-VL | | GTPARFSGSLSGGKAALTLSGVQPEDEAEYYCTLWYSNRWVFGSGTKLTVL |
| 134 | 2B2_Comp1_ | artificial | KYAMN |
| | I2K-HCDR1 | | |
| 135 | 2B2_Comp1_ | artificial | RIRSKYNNYATYYAEAVKG |
| | I2K-HCDR2 | | |
| 136 | 2B2_Comp1_ | artificial | NENIGTSYISYWAY |
| | I2K-HCDR3 | | |
| 137 | 2B2_Comp1_ | artificial | ASSTGAVTSGNYPN |
| | I2K-LCDR1 | | |
| 138 | 2B2_Comp1_ | artificial | GTKFLVP |
| | I2K-LCDR2 | | |
| 139 | 2B2_Comp1_ | artificial | TLWYSNRWV |
| | I2K-LCDR3 | | |
| 140 | 2B2_Comp1_ | artificial | EVQLVESGGGLVQPGGSLKLSCAASGFTFNKYAMNWVRQAPGKGLEWVARIRSKYNNY |
| | 2K-VH | | ATYYAEAVKGRFTISRDDSKNTVYLQMNNLKTEDTAVYYCVRNENIGTSYISYWAYWG |
| | | | QGTLVTVSS |
| 141 | 2B2_Comp1_ | artificial | QTVVTQEPSLTVSPGGTVTITCASSTGAVTSGNYPNWVQQKPGQAPRGLIGGTKFLVP |
| | I2K-VL | | GTPARFSGSLSGGKAALTLSGVQPEDEAEYYCTLWYSNRWVFGSGTKLTVL |
| 142 | 2B2_Comp1_ | artificial | KYAIN |
| | I2L-HCDR1 | | |
| 143 | 2B2_Comp1_ | artificial | RIRSKYNNYATYYADAVKD |
| | I2L-HCDR2 | | |
| 144 | 2B2_Comp1_ | artificial | AANFGSSYISYFAY |
| | I2L-HCDR3 | | |
| 145 | 2B2_Comp1_ | artificial | ASSTGAVTSGNYPN |
| | I2L-LCDR1 | | |
| 146 | 2B2_Comp1_ | artificial | GTKFLVP |
| | I2L-LCDR2 | | |
| 147 | 2B2_Comp1_ | artificial | TLYYSNRWV |
| | I2L-LCDR3 | | |
| 148 | 2B2_Comp1_ | artificial | EVQLVESGGGLVQPGGSLKLSCAASGFTENKYAINWVRQAPGKGMEWVARIRSKYNNY |
| | I2L-VH | | ATYYADAVKDRFTISRDDSKNTLYLQMNNLKTEDTAVYYCVRAANFGSSYISYFAYWG |
| | | | QGTLVTVSS |
| 149 | 2B2_Comp1_ | artificial | QTVVTQEPSLTVSPGGTVTITCASSTGAVTSGNYPNWIQKKPGQAPRGLIGGTKFLVP |
| | I2L-VL | | GTPARFSGSLEGGKAALTLSGVQPEDEAEYYCTLYYSNRWVFGSGTKLTVL |
| 150 | 2B2_Comp1_ | artificial | KYAIN |
| | I2M-HCDR1 | | |
| 151 | 2B2_Comp1_ | artificial | RIRSKYNNYATYYADAVKD |
| | I2M-HCDR2 | | |
| 152 | 2B2_Comp1_ | artificial | AANFGTSYISYWAY |
| | I2M-HCDR3 | | |
| 153 | 2B2_Comp1_ | artificial | ASSTGAVTSGNYPN |
| | I2M-LCDR1 | | |
| 154 | 2B2_Comp1_ | artificial | GTKFLVP |
| | I2M-LCDR2 | | |
| 155 | 2B2_Comp1_ | artificial | TLWYSNRWV |
| | I2M-LCDR3 | | |
| 156 | 2B2_Comp1_ | artificial | EVQLVESGGGLVQPGGSLKLSCAASGFTFNKYAINWVRQAPGKGMEWVARIRSKYNNY |
| | I2M-VH | | ATYYADAVKDRFTISRDDSKNTLYLQMNNLKTEDTAVYYCVRAANFGTSYISYWAYWG |
| | | | QGTLVTVSS |
| 157 | 2B2_Comp1_ | artificial | QTVVTQEPSLTVSPGGTVTITCASSTGAVTSGNYPNWVQKKPGQAPRGLIGGTKFLVP |
| | I2M-VL | | GTPARFSGSLSGGKAALTLSGVQPEDEAEYYCTLWYSNRWVFGSGTKLTVL |
| 158 | 2B2_Comp1_ | artificial | KYAIN |
| | I2M2-HCDR1 | | |
| 159 | 2B2_Comp1_ | artificial | RIRSKYNNYATYYADAVKD |
| | I2M2-HCDR2 | | |
| 160 | 2B2_Comp1_ | artificial | NANFGTSYISYFAY |
| | I2M2-HCDR3 | | |
| 161 | 2B2_Comp1_ | artificial | ASSTGAVTSGNYPN |
| | I2M2-LCDR1 | | |
| 162 | 2B2_Comp1_ | artificial | GTKFLVP |
| | I2M2-LCDR2 | | |
| 163 | 2B2_Comp1_ | artificial | TLWYSNRWV |
| | I2M2-LCDR3 | | |
| 164 | 2B2_Comp1_ | artificial | EVQLVESGGGLVQPGGSLKLSCAASGFTFNKYAINWVREAPGKGLEWVARIRSKYNNY |
| | I2M2-VH | | ATYYADAVKDRFTISRDDSKNTAYLQMNNLKTEDTAVYYCVRNANFGTSYISYFAYWG |
| | | | QGTLVTVSS |
| 165 | 2B2_Comp1_ | artificial | QTVVTQEPSLTVSPGGTVTLTCASSTGAVTSGNYPNWVQKKPGQAPRGLIGGTKFLVP |
| | I2M2-VL | | GTPARFSGSLLGGKAALTLSGVQPEDEAEYYCTLWYSNRWVFGSGTKLTVL |
| 166 | A2J-HCDR1 | artificial | VYAMN |
| 167 | A2J-HCDR2 | artificial | RIRSKYNNYATYYADSVKK |
| 168 | A2J-HCDR3 | artificial | HGNFGNSYLSWWAY |
| 169 | A2J-LCDR1 | artificial | RSSTGAVTSGYYPN |
| 170 | A2J-LCDR2 | artificial | ATDMRPS |
| 171 | A2J-LCDR3 | artificial | ALWYSNRWV |
| 172 | A2J-VH | artificial | EVQLVESGGGLVQPGGSLKLSCAASGFTFNVYAMNWVRQAPGKGLEWVARIRSKYNNY |
| | | | ATYYADSVKKRFTISRDDSKNTAYLQMNNLKTEDTAVYYCVRHGNFGNSYLSWWAYWG |
| | | | QGTLVTVSS |
| 173 | A2J-VL | artificial | QTVVTQEPSLTVSPGGTVTLTCRSSTGAVTSGYYPNWVQQKPGQAPRGLIGATDMRPS |
| | | | GTPARFSGSLLGGKAALTLSGVQPEDEAEYYCALWYSNRWVFGGGTKLTVL |
| 174 | A2J_HC_A89I | artificial | EVQLVESGGGLVQPGGSLKLSCAASGFTFNVYAMNWVRQAPGKGLEWVARIRSKYNNY |
| | -VH | | ATYYADSVKKRFTISRDDSKNTIYLQMNNLKTEDTAVYYCVRHGNFGNSYLSWWAYWG |
| | | | QGTLVTVSS |
| 175 | A2J_HC_A89I | artificial | QTVVTQEPSLTVSPGGTVTLTCRSSTGAVTSGYYPNWVQQKPGQAPRGLIGATDMRPS |
| | -VL | | GTPARFSGSLLGGKAALTLSGVQPEDEAEYYCALWYSNRWVFGGGTKLTVL |
| 176 | A2J_HC_A89L | artificial | EVQLVESGGGLVQPGGSLKLSCAASGFTFNVYAMNWVRQAPGKGLEWVARIRSKYNNY |
| | -VH | | ATYYADSVKKRFTISRDDSKNTLYLQMNNLKTEDTAVYYCVRHGNFGNSYLSWWAYWG |
| | | | QGTLVTVSS |
| 177 | A2J_HC_A89L | artificial | QTVVTQEPSLTVSPGGTVTLTCRSSTGAVTSGYYPNWVQQKPGQAPRGLIGATDMRPS |
| | -VL | | GTPARFSGSLLGGKAALTLSGVQPEDEAEYYCALWYSNRWVFGGGTKLTVL |
| 178 | A2J_HC_A89T | artificial | EVQLVESGGGLVQPGGSLKLSCAASGFTFNVYAMNWVRQAPGKGLEWVARIRSKYNNY |
| | -VH | | ATYYADSVKKRFTISRDDSKNTTYLQMNNLKTEDTAVYYCVRHGNFGNSYLSWWAYWG |
| | | | QGTLVTVSS |
| 179 | A2J_HC_A89T | artificial | QTVVTQEPSLTVSPGGTVTLTCRSSTGAVTSGYYPNWVQQKPGQAPRGLIGATDMRPS |
| | -VL | | GTPARFSGSLLGGKAALTLSGVQPEDEAEYYCALWYSNRWVFGGGTKLTVL |
| 180 | A2J_HC_A89V | artificial | EVQLVESGGGLVQPGGSLKLSCAASGFTFNVYAMNWVRQAPGKGLEWVARIRSKYNNY |
| | -VH | | ATYYADSVKKRFTISRDDSKNTVYLQMNNLKTEDTAVYYCVRHGNFGNSYLSWWAYWG |
| | | | QGTLVTVSS |
| 181 | A2J_HC_A89V | artificial | QTVVTQEPSLTVSPGGTVTLTCRSSTGAVTSGYYPNWVQQKPGQAPRGLIGATDMRPS |
| | -VL | | GTPARFSGSLLGGKAALTLSGVQPEDEAEYYCALWYSNRWVFGGGTKLTVL |
| 182 | A2J_HC_L52M | artificial | EVQLVESGGGLVQPGGSLKLSCAASGFTFNVYAMNWVRQAPGKGMEWVARIRSKYNNY |
| | -VH | | ATYYADSVKKRFTISRDDSKNTAYLQMNNLKTEDTAVYYCVRHGNFGNSYLSWWAYWG |
| | | | QGTLVTVSS |
| 183 | A2J_HC_L52M | artificial | QTVVTQEPSLTVSPGGTVTLTCRSSTGAVTSGYYPNWVQQKPGQAPRGLIGATDMRPS |
| | -VL | | GTPARFSGSLLGGKAALTLSGVQPEDEAEYYCALWYSNRWVFGGGTKLTVL |
| 184 | A2J_HC_L52M_ | artificial | EVQLVESGGGLVQPGGSLKLSCAASGFTFNVYAMNWVRQAPGKGMEWVARIRSKYNNY |
| | LC_L21I_Q46K_ | | ATYYADSVKKRFTISRDDSKNTAYLQMNNLKTEDTAVYYCVRHGNFGNSYLSWWAYWG |
| | G141S- | | QGTLVTVSS |
| | VH | | |
| 185 | A2J_HC_L52M_ | artificial | QTVVTQEPSLTVSPGGTVTITCRSSTGAVTSGYYPNWVQKKPGQAPRGLIGATDMRPS |
| | LC_L21I_Q46K_ | | GTPARFSGSLLGGKAALTLSGVQPEDEAEYYCALWYSNRWVFGSGTKLTVL |
| | G141S- | | |
| | VL | | |
| 186 | A2J_HC_L52M_ | artificial | EVQLVESGGGLVQPGGSLKLSCAASGFTFNVYAMNWVRQAPGKGMEWVARIRSKYNNY |
| | LC_Q46K- | | ATYYADSVKKRFTISRDDSKNTAYLQMNNLKTEDTAVYYCVRHGNFGNSYLSWWAYWG |
| | VH | | QGTLVTVSS |
| 187 | A2J_HC_L52M_ | artificial | QTVVTQEPSLTVSPGGTVTLTCRSSTGAVTSGYYPNWVQKKPGQAPRGLIGATDMRPS |
| | LC_Q46K- | | GTPARFSGSLLGGKAALTLSGVQPEDEAEYYCALWYSNRWVFGGGTKLTVL |
| | VL | | |
| 188 | A2J_HC_L52M_ | artificial | EVQLVESGGGLVQPGGSLKLSCAASGFTFNVYAMNWVRQAPGKGMEWVARIRSKYNNY |
| | LC_Q46K_ | | ATYYADSVKKRFTISRDDSKNTAYLQMNNLKTEDTAVYYCVRHGNFGNSYLSWWAYWG |
| | G141S-VH | | QGTLVTVSS |
| 189 | A2J_HC_L52M_ | artificial | QTVVTQEPSLTVSPGGTVTLTCRSSTGAVTSGYYPNWVQKKPGQAPRGLIGATDMRPS |
| | LC_Q46K_ | | GTPARFSGSLLGGKAALTLSGVQPEDEAEYYCALWYSNRWVFGSGTKLTVL |
| | G141S-VL | | |
| 190 | A2J_HC_L52V | artificial | EVQLVESGGGLVQPGGSLKLSCAASGFTFNVYAMNWVRQAPGKGVEWVARIRSKYNNY |
| | -VH | | ATYYADSVKKRFTISRDDSKNTAYLQMNNLKTEDTAVYYCVRHGNFGNSYLSWWAYWG |
| | | | QGTLVTVSS |
| 191 | A2J_HC_L52V | artificial | QTVVTQEPSLTVSPGGTVTLTCRSSTGAVTSGYYPNWVQQKPGQAPRGLIGATDMRPS |
| | -VL | | GTPARFSGSLLGGKAALTLSGVQPEDEAEYYCALWYSNRWVFGGGTKLTVL |
| 192 | A2J_HC_L52V_ | artificial | EVQLVESGGGLVQPGGSLKLSCAASGFTFNVYAMNWVRQAPGKGVEWVARIRSKYNNY |
| | LC_V44F- | | ATYYADSVKKRFTISRDDSKNTAYLQMNNLKTEDTAVYYCVRHGNFGNSYLSWWAYWG |
| | VH | | QGTLVTVSS |
| 193 | A2J_HC_L52V_ | artificial | QTVVTQEPSLTVSPGGTVTLTCRSSTGAVTSGYYPNWFQQKPGQAPRGLIGATDMRPS |
| | LC_V44F- | | GTPARFSGSLLGGKAALTLSGVQPEDEAEYYCALWYSNRWVFGGGTKLTVL |
| | VL | | |
| 194 | A2J_HC_L52V_ | artificial | EVQLVESGGGLVQPGGSLKLSCAASGFTFNVYAMNWVRQAPGKGVEWVARIRSKYNNY |
| | LC_V44Y- | | ATYYADSVKKRFTISRDDSKNTAYLQMNNLKTEDTAVYYCVRHGNFGNSYLSWWAYWG |
| | VH | | QGTLVTVSS |
| 195 | A2J_HC_L52V_ | artificial | QTVVTQEPSLTVSPGGTVTLTCRSSTGAVTSGYYPNWYQQKPGQAPRGLIGATDMRPS |
| | LC_V44Y- | | GTPARFSGSLLGGKAALTLSGVQPEDEAEYYCALWYSNRWVFGGGTKLTVL |
| | VL | | |
| 196 | A2J_HC_M41F_ | artificial | VYAFN |
| | A89V- | | |
| | HCDR1 | | |
| 197 | A2J_HC_M41F_ | artificial | RIRSKYNNYATYYADSVKK |
| | A89V-HCDR2 | | |
| 198 | A2J_HC_M41F_ | artificial | HGNFGNSYLSWWAY |
| | A89V-HCDR3 | | |
| 199 | A2J_HC_M41F_ | artificial | RSSTGAVTSGYYPN |
| | A89V- | | |
| | LCDR1 | | |
| 200 | A2J_HC_M41F | artificial | ATDMRPS |
| | A89V-LCDR2 | | |
| 201 | A2J_HC_M41F_ | artificial | ALWYSNRWV |
| | A89V-LCDR3 | | |
| 202 | A2J_HC_M41F_ | artificial | EVQLVESGGGLVQPGGSLKLSCAASGFTFNVYAFNWVRQAPGKGLEWVARIRSKYNNY |
| | A89V-VH | | ATYYADSVKKRFTISRDDSKNTVYLQMNNLKTEDTAVYYCVRHGNFGNSYLSWWAYWG |
| | | | QGTLVTVSS |
| 203 | A2J_HC_M41F_ | artificial | QTVVTQEPSLTVSPGGTVTLTCRSSTGAVTSGYYPNWVQQKPGQAPRGLIGATDMRPS |
| | A89V-VL | | GTPARFSGSLLGGKAALTLSGVQPEDEAEYYCALWYSNRWVFGGGTKLTVL |
| 204 | A2J_HC_M41I | artificial | VYAIN |
| | -HCDR1 | | |
| 205 | A2J_HC_M41I | artificial | RIRSKYNNYATYYADSVKK |
| | -HCDR2 | | |
| 206 | A2J_HC_M41I | artificial | HGNFGNSYLSWWAY |
| | -HCDR3 | | |
| 207 | A2J_HC_M41I | artificial | RSSTGAVTSGYYPN |
| | -LCDR1 | | |
| 208 | A2J_HC_M41I | artificial | ATDMRPS |
| | -LCDR2 | | |
| 209 | A2J_HC_M41I | artificial | ALWYSNRWV |
| | -LCDR3 | | |
| 210 | A2J_HC_M41I | artificial | EVQLVESGGGLVQPGGSLKLSCAASGFTFNVYAINWVRQAPGKGLEWVARIRSKYNNY |
| | -VH | | ATYYADSVKKRFTISRDDSKNTAYLQMNNLKTEDTAVYYCVRHGNFGNSYLSWWAYWG |
| | | | QGTLVTVSS |
| 211 | A2J_HC_M41I | artificial | QTVVTQEPSLTVSPGGTVTLTCRSSTGAVTSGYYPNWVQQKPGQAPRGLIGATDMRPS |
| | -VL | | GTPARFSGSLLGGKAALTLSGVQPEDEAEYYCALWYSNRWVFGGGTKLTVL |
| 212 | A2J_HC_M41I_ | artificial | EVQLVESGGGLVQPGGSLKLSCAASGFTFNVYAINWVRQAPGKGLEWVARIRSKYNNY |
| | A89L-VH | | ATYYADSVKKRFTISRDDSKNTLYLQMNNLKTEDTAVYYCVRHGNFGNSYLSWWAYWG |
| | | | QGTLVTVSS |
| 213 | A2J_HC_M41I_ | artificial | QTVVTQEPSLTVSPGGTVTLTCRSSTGAVTSGYYPNWVQQKPGQAPRGLIGATDMRPS |
| | A89L-VL | | GTPARFSGSLLGGKAALTLSGVQPEDEAEYYCALWYSNRWVFGGGTKLTVL |
| 214 | A2J_HC_M41I_ | artificial | EVQLVESGGGLVQPGGSLKLSCAASGFTFNVYAINWVRQAPGKGLEWVARIRSKYNNY |
| | A89V-VH | | ATYYADSVKKRFTISRDDSKNTVYLQMNNLKTEDTAVYYCVRHGNFGNSYLSWWAYWG |
| | | | QGTLVTVSS |
| 215 | A2J_HC_M41I_ | artificial | QTVVTQEPSLTVSPGGTVTLTCRSSTGAVTSGYYPNWVQQKPGQAPRGLIGATDMRPS |
| | A89V-VL | | GTPARFSGSLLGGKAALTLSGVQPEDEAEYYCALWYSNRWVFGGGTKLTVL |
| 216 | A2J_HC_M41I_ | artificial | EVQLVESGGGLVQPGGSLKLSCAASGFTFNVYAINWVRQAPGKGLEWVARIRSKYNNY |
| | A89V_LC_L21I_ | | ATYYADSVKKRFTISRDDSKNTVYLQMNNLKTEDTAVYYCVRHGNFGNSYLSWWAYWG |
| | Q46K_ | | QGTLVTVSS |
| | G141S-VH | | |
| 217 | A2J_HC_M41I_ | artificial | QTVVTQEPSLTVSPGGTVTITCRSSTGAVTSGYYPNWVQKKPGQAPRGLIGATDMRPS |
| | A89V_LC_L21I_ | | GTPARFSGSLLGGKAALTLSGVQPEDEAEYYCALWYSNRWVFGSGTKLTVL |
| | Q46K_ | | |
| | G141S-VL | | |
| 218 | A2J_HC_M41I_ | artificial | EVQLVESGGGLVQPGGSLKLSCAASGFTFNVYAINWVRQAPGKGLEWVARIRSKYNNY |
| | A89V_LC_ | | ATYYADSVKKRFTISRDDSKNTVYLQMNNLKTEDTAVYYCVRHGNFGNSYLSWWAYWG |
| | Q46K-VH | | QGTLVTVSS |
| 219 | A2J_HC_M41I_ | artificial | QTVVTQEPSLTVSPGGTVTLTCRSSTGAVTSGYYPNWVQKKPGQAPRGLIGATDMRPS |
| | A89V_LC_ | | GTPARFSGSLLGGKAALTLSGVQPEDEAEYYCALWYSNRWVFGGGTKLTVL |
| | Q46K-VL | | |
| 220 | A2J_HC_M41I_ | artificial | EVQLVESGGGLVQPGGSLKLSCAASGFTFNVYAINWVRQAPGKGVEWVARIRSKYNNY |
| | L52V_A89V_ | | ATYYADSVKKRFTISRDDSKNTVYLQMNNLKTEDTAVYYCVRHGNFGNSYLSWWAYWG |
| | LC_L21I_ | | QGTLVTVSS |
| | Q46K_G141S_ | | |
| | V44F-VH | | |
| 221 | A2J_HC_M41I_ | artificial | QTVVTQEPSLTVSPGGTVTITCRSSTGAVTSGYYPNWFQKKPGQAPRGLIGATDMRPS |
| | L52V_A89V_ | | GTPARFSGSLLGGKAALTLSGVQPEDEAEYYCALWYSNRWVFGSGTKLTVL |
| | LC_L21I_ | | |
| | Q46K_G141S- | | |
| | V44F-VL | | |
| 222 | A2J_HC_M41I_ | artificial | EVQLVESGGGLVQPGGSLKLSCAASGFTFNVYAINWVRQAPGKGLEWVARIRSKYNNY |
| | LC_L21I_ | | ATYYADSVKKRFTISRDDSKNTAYLQMNNLKTEDTAVYYCVRHGNFGNSYLSWWAYWG |
| | Q46K_G141S- | | QGTLVTVSS |
| | VH | | |
| 223 | A2J_HC_M41I_ | artificial | QTVVTQEPSLTVSPGGTVTITCRSSTGAVTSGYYPNWVQKKPGQAPRGLIGATDMRPS |
| | LC_L21I_ | | GTPARFSGSLLGGKAALTLSGVQPEDEAEYYCALWYSNRWVFGSGTKLTVL |
| | Q46K_G141S- | | |
| | VL | | |
| 224 | A2J_HC_M41I_ | artificial | EVQLVESGGGLVQPGGSLKLSCAASGFTFNVYAINWVREAPGKGLEWVARIRSKYNNY |
| | Q46E_A89V_ | | ATYYADSVKKRFTISRDDSKNTVYLQMNNLKTEDTAVYYCVRHGNFGNSYLSWWAYWG |
| | LC_L21I_ | | QGTLVTVSS |
| | Q46K_G141S- | | |
| | VH | | |
| 225 | A2J_HC_M41I_ | artificial | QTVVTQEPSLTVSPGGTVTITCRSSTGAVTSGYYPNWVQKKPGQAPRGLIGATDMRPS |
| | Q46E_A89V_ | | GTPARFSGSLLGGKAALTLSGVQPEDEAEYYCALWYSNRWVFGSGTKLTVL |
| | LC_L21I_ | | |
| | Q46K_G141S- | | |
| | VL | | |
| 226 | A2J_HC_M41V | artificial | VYAVN |
| | -HCDR1 | | |
| 227 | A2J_HC_M41V | artificial | RIRSKYNNYATYYADSVKK |
| | -HCDR2 | | |
| 228 | A2J_HC_M41V | artificial | HGNFGNSYLSWWAY |
| | -HCDR3 | | |
| 229 | A2J_HC_M41V | artificial | RSSTGAVTSGYYPN |
| | -LCDR1 | | |
| 230 | A2J_HC_M41V | artificial | ATDMRPS |
| | -LCDR2 | | |
| 231 | A2J_HC_M41V | artificial | ALWYSNRWV |
| | -LCDR3 | | |
| 232 | A2J_HC_M41V | artificial | EVQLVESGGGLVQPGGSLKLSCAASGFTFNVYAVNWVRQAPGKGLEWVARIRSKYNNY |
| | -VH | | ATYYADSVKKRFTISRDDSKNTAYLQMNNLKTEDTAVYYCVRHGNFGNSYLSWWAYWG |
| | | | QGTLVTVSS |
| 233 | A2J_HC_M41V | artificial | QTVVTQEPSLTVSPGGTVTLTCRSSTGAVTSGYYPNWVQQKPGQAPRGLIGATDMRPS |
| | -VL | | GTPARFSGSLLGGKAALTLSGVQPEDEAEYYCALWYSNRWVFGGGTKLTVL |
| 234 | A2J_HC_Q46D | artificial | EVQLVESGGGLVQPGGSLKLSCAASGFTFNVYAMNWVRDAPGKGLEWVARIRSKYNNY |
| | -VH | | ATYYADSVKKRFTISRDDSKNTAYLQMNNLKTEDTAVYYCVRHGNFGNSYLSWWAYWG |
| | | | QGTLVTVSS |
| 235 | A2J_HC_Q46D | artificial | QTVVTQEPSLTVSPGGTVTLTCRSSTGAVTSGYYPNWVQQKPGQAPRGLIGATDMRPS |
| | -VL | | GTPARFSGSLLGGKAALTLSGVQPEDEAEYYCALWYSNRWVFGGGTKLTVL |
| 236 | A2J_HC_Q46D_ | artificial | EVQLVESGGGLVQPGGSLKLSCAASGFTFNVYAMNWVRDAPGKGLEWVARIRSKYNNY |
| | LC_L21I_ | | ATYYADSVKKRFTISRDDSKNTAYLQMNNLKTEDTAVYYCVRHGNFGNSYLSWWAYWG |
| | Q46K_G141S- | | QGTLVTVSS |
| | VH | | |
| 237 | A2J_HC_Q46D_ | artificial | QTVVTQEPSLTVSPGGTVTITCRSSTGAVTSGYYPNWVQKKPGQAPRGLIGATDMRPS |
| | LC_L21I_ | | GTPARFSGSLLGGKAALTLSGVQPEDEAEYYCALWYSNRWVFGSGTKLTVL |
| | Q46K_G141S- | | |
| | VL | | |
| 238 | A2J_HC_Q46D_ | artificial | EVQLVESGGGLVQPGGSLKLSCAASGFTFNVYAMNWVRDAPGKGLEWVARIRSKYNNY |
| | LC_L21I_ | | ATYYADSVKKRFTISRDDSKNTAYLQMNNLKTEDTAVYYCVRHGNFGNSYLSWWAYWG |
| | Q46R_G141S- | | QGTLVTVSS |
| | VH | | |
| 239 | A2J_HC_Q46D_ | artificial | QTVVTQEPSLTVSPGGTVTITCRSSTGAVTSGYYPNWVQRKPGQAPRGLIGATDMRPS |
| | LC_L21I_ | | GTPARFSGSLLGGKAALTLSGVQPEDEAEYYCALWYSNRWVFGSGTKLTVL |
| | Q46R_G141S- | | |
| | VL | | |
| 240 | A2J_HC_Q46D_ | artificial | EVQLVESGGGLVQPGGSLKLSCAASGFTFNVYAMNWVRDAPGKGLEWVARIRSKYNNY |
| | LC_Q46K- | | ATYYADSVKKRFTISRDDSKNTAYLQMNNLKTEDTAVYYCVRHGNFGNSYLSWWAYWG |
| | VH | | QGTLVTVSS |
| 241 | A2J_HC_Q46D_ | artificial | QTVVTQEPSLTVSPGGTVTLTCRSSTGAVTSGYYPNWVQKKPGQAPRGLIGATDMRPS |
| | LC_Q46K- | | GTPARFSGSLLGGKAALTLSGVQPEDEAEYYCALWYSNRWVFGGGTKLTVL |
| | VL | | |
| 242 | A2J_HC_Q46D_ | artificial | EVQLVESGGGLVQPGGSLKLSCAASGFTFNVYAMNWVRDAPGKGLEWVARIRSKYNNY |
| | LC_Q46K_ | | ATYYADSVKKRFTISRDDSKNTAYLQMNNLKTEDTAVYYCVRHGNFGNSYLSWWAYWG |
| | G141S-VH | | QGTLVTVSS |
| 243 | A2J_HC_Q46D_ | artificial | QTVVTQEPSLTVSPGGTVTLTCRSSTGAVTSGYYPNWVQKKPGQAPRGLIGATDMRPS |
| | LC_Q46K_ | | GTPARFSGSLLGGKAALTLSGVQPEDEAEYYCALWYSNRWVFGSGTKLTVL |
| | G141S-VL | | |
| 244 | A2J_HC_Q46D_ | artificial | EVQLVESGGGLVQPGGSLKLSCAASGFTFNVYAMNWVRDAPGKGLEWVARIRSKYNNY |
| | LC_Q46R- | | ATYYADSVKKRFTISRDDSKNTAYLQMNNLKTEDTAVYYCVRHGNFGNSYLSWWAYWG |
| | VH | | QGTLVTVSS |
| 245 | A2J_HC_Q46D_ | artificial | QTVVTQEPSLTVSPGGTVTLTCRSSTGAVTSGYYPNWVQRKPGQAPRGLIGATDMRPS |
| | LC_Q46R- | | GTPARFSGSLLGGKAALTLSGVQPEDEAEYYCALWYSNRWVFGGGTKLTVL |
| | VL | | |
| 246 | A2J_HC_Q46D_ | artificial | EVQLVESGGGLVQPGGSLKLSCAASGFTFNVYAMNWVRDAPGKGLEWVARIRSKYNNY |
| | LC_Q46R_ | | ATYYADSVKKRFTISRDDSKNTAYLQMNNLKTEDTAVYYCVRHGNFGNSYLSWWAYWG |
| | G141S-VH | | QGTLVTVSS |
| 247 | A2J_HC_Q46D_ | artificial | QTVVTQEPSLTVSPGGTVTLTCRSSTGAVTSGYYPNWVQRKPGQAPRGLIGATDMRPS |
| | LC_Q46R_ | | GTPARFSGSLLGGKAALTLSGVQPEDEAEYYCALWYSNRWVFGSGTKLTVL |
| | G141S-VL | | |
| 248 | A2J_HC_Q46E | artificial | EVQLVESGGGLVQPGGSLKLSCAASGFTFNVYAMNWVREAPGKGLEWVARIRSKYNNY |
| | -VH | | ATYYADSVKKRFTISRDDSKNTAYLQMNNLKTEDTAVYYCVRHGNFGNSYLSWWAYWG |
| | | | QGTLVTVSS |
| 249 | A2J_HC_Q46E | artificial | QTVVTQEPSLTVSPGGTVTLTCRSSTGAVTSGYYPNWVQQKPGQAPRGLIGATDMRPS |
| | -VL | | GTPARFSGSLLGGKAALTLSGVQPEDEAEYYCALWYSNRWVFGGGTKLTVL |
| 250 | A2J_HC_Q46E_ | artificial | EVQLVESGGGLVQPGGSLKLSCAASGFTFNVYAMNWVREAPGKGLEWVARIRSKYNNY |
| | LC_L21I_ | | ATYYADSVKKRFTISRDDSKNTAYLQMNNLKTEDTAVYYCVRHGNFGNSYLSWWAYWG |
| | Q46K_G141S- | | QGTLVTVSS |
| | VH | | |
| 251 | A2J_HC_Q46E_ | artificial | QTVVTQEPSLTVSPGGTVTITCRSSTGAVTSGYYPNWVQKKPGQAPRGLIGATDMRPS |
| | LC_L21I_ | | GTPARFSGSLLGGKAALTLSGVQPEDEAEYYCALWYSNRWVFGSGTKLTVL |
| | Q46K_G141S | | |
| | VL | | |
| 252 | A2J_HC_Q46E_ | artificial | EVQLVESGGGLVQPGGSLKLSCAASGFTFNVYAMNWVREAPGKGLEWVARIRSKYNNY |
| | LC_L21I_ | | ATYYADSVKKRFTISRDDSKNTAYLQMNNLKTEDTAVYYCVRHGNFGNSYLSWWAYWG |
| | Q46R_G141S- | | QGTLVTVSS |
| | VH | | |
| 253 | A2J_HC_Q46E_ | artificial | QTVVTQEPSLTVSPGGTVTITCRSSTGAVTSGYYPNWVQRKPGQAPRGLIGATDMRPS |
| | LC_L21I_ | | GTPARFSGSLLGGKAALTLSGVQPEDEAEYYCALWYSNRWVFGSGTKLTVL |
| | Q46R_G141S- | | |
| | VL | | |
| 254 | A2J_HC_Q46E_ | artificial | EVQLVESGGGLVQPGGSLKLSCAASGFTFNVYAMNWVREAPGKGLEWVARIRSKYNNY |
| | LC_Q46K- | | ATYYADSVKKRFTISRDDSKNTAYLQMNNLKTEDTAVYYCVRHGNFGNSYLSWWAYWG |
| | VH | | QGTLVTVSS |
| 255 | A2J_HC_Q46E_ | artificial | QTVVTQEPSLTVSPGGTVTLTCRSSTGAVTSGYYPNWVQKKPGQAPRGLIGATDMRPS |
| | LC_Q46K- | | GTPARFSGSLLGGKAALTLSGVQPEDEAEYYCALWYSNRWVFGGGTKLTVL |
| | VL | | |
| 256 | A2J_HC_Q46E_ | artificial | EVQLVESGGGLVQPGGSLKLSCAASGFTFNVYAMNWVREAPGKGLEWVARIRSKYNNY |
| | LC_Q46K_ | | ATYYADSVKKRFTISRDDSKNTAYLQMNNLKTEDTAVYYCVRHGNFGNSYLSWWAYWG |
| | G141S-VH | | QGTLVTVSS |
| 257 | A2J_HC_Q46E_ | artificial | QTVVTQEPSLTVSPGGTVTLTCRSSTGAVTSGYYPNWVQKKPGQAPRGLIGATDMRPS |
| | LC_Q46K_ | | GTPARFSGSLLGGKAALTLSGVQPEDEAEYYCALWYSNRWVFGSGTKLTVL |
| | G141S-VL | | |
| 258 | A2J_HC_Q46E_ | artificial | EVQLVESGGGLVQPGGSLKLSCAASGFTFNVYAMNWVREAPGKGLEWVARIRSKYNNY |
| | LC_Q46R- | | ATYYADSVKKRFTISRDDSKNTAYLQMNNLKTEDTAVYYCVRHGNFGNSYLSWWAYWG |
| | VH | | QGTLVTVSS |
| 259 | A2J_HC_Q46E_ | artificial | QTVVTQEPSLTVSPGGTVTLTCRSSTGAVTSGYYPNWVQRKPGQAPRGLIGATDMRPS |
| | LC_Q46R- | | GTPARFSGSLLGGKAALTLSGVQPEDEAEYYCALWYSNRWVFGGGTKLTVL |
| | VL | | |
| 260 | A2J_HC_Q46E_ | artificial | EVQLVESGGGLVQPGGSLKLSCAASGFTFNVYAMNWVREAPGKGLEWVARIRSKYNNY |
| | LC_Q46R_ | | ATYYADSVKKRFTISRDDSKNTAYLQMNNLKTEDTAVYYCVRHGNFGNSYLSWWAYWG |
| | G141S-VH | | QGTLVTVSS |
| 261 | A2J_HC_Q46E_ | artificial | QTVVTQEPSLTVSPGGTVTLTCRSSTGAVTSGYYPNWVQRKPGQAPRGLIGATDMRPS |
| | LC_Q46R_ | | GTPARFSGSLLGGKAALTLSGVQPEDEAEYYCALWYSNRWVFGSGTKLTVL |
| | G141S-VL | | |
| 262 | A2J_HC_Q46K | artificial | EVQLVESGGGLVQPGGSLKLSCAASGFTFNVYAMNWVRKAPGKGLEWVARIRSKYNNY |
| | -VH | | ATYYADSVKKRFTISRDDSKNTAYLQMNNLKTEDTAVYYCVRHGNFGNSYLSWWAYWG |
| | | | QGTLVTVSS |
| 263 | A2J_HC_Q46K | artificial | QTVVTQEPSLTVSPGGTVTLTCRSSTGAVTSGYYPNWVQQKPGQAPRGLIGATDMRPS |
| | -VL | | GTPARFSGSLLGGKAALTLSGVQPEDEAEYYCALWYSNRWVFGGGTKLTVL |
| 264 | A2J_HC_Q46R | artificial | EVQLVESGGGLVQPGGSLKLSCAASGFTFNVYAMNWVRRAPGKGLEWVARIRSKYNNY |
| | -VH | | ATYYADSVKKRFTISRDDSKNTAYLQMNNLKTEDTAVYYCVRHGNFGNSYLSWWAYWG |
| | | | QGTLVTVSS |
| 265 | A2J_HC_Q46R | artificial | QTVVTQEPSLTVSPGGTVTLTCRSSTGAVTSGYYPNWVQQKPGQAPRGLIGATDMRPS |
| | -VL | | GTPARFSGSLLGGKAALTLSGVQPEDEAEYYCALWYSNRWVFGGGTKLTVL |
| 266 | A2J_HC_V107A- | artificial | EVQLVESGGGLVQPGGSLKLSCAASGFTFNVYAMNWVRQAPGKGLEWVARIRSKYNNY |
| | VH | | ATYYADSVKKRFTISRDDSKNTAYLQMNNLKTEDTAVYYCARHGNFGNSYLSWWAYWG |
| | | | QGTLVTVSS |
| 267 | A2J_HC_V107A- | artificial | QTVVTQEPSLTVSPGGTVTLTCRSSTGAVTSGYYPNWVQQKPGQAPRGLIGATDMRPS |
| | VL | | GTPARFSGSLLGGKAALTLSGVQPEDEAEYYCALWYSNRWVFGGGTKLTVL |
| 268 | A2J_HC_V107L- | artificial | EVQLVESGGGLVQPGGSLKLSCAASGFTFNVYAMNWVRQAPGKGLEWVARIRSKYNNY |
| | VH | | ATYYADSVKKRFTISRDDSKNTAYLQMNNLKTEDTAVYYCLRHGNFGNSYLSWWAYWG |
| | | | QGTLVTVSS |
| 269 | A2J_HC_V107L- | artificial | QTVVTQEPSLTVSPGGTVTLTCRSSTGAVTSGYYPNWVQQKPGQAPRGLIGATDMRPS |
| | VL | | GTPARFSGSLLGGKAALTLSGVQPEDEAEYYCALWYSNRWVFGGGTKLTVL |
| 270 | A2J_HC_W136F- | artificial | VYAMN |
| | HCDR1 | | |
| 271 | A2J_HC_W136F- | artificial | RIRSKYNNYATYYADSVKK |
| | HCDR2 | | |
| 272 | A2J_HC_W136F- | artificial | HGNFGNSYLSWFAY |
| | HCDR3 | | |
| 273 | A2J_HC_W136F- | artificial | RSSTGAVTSGYYPN |
| | LCDR1 | | |
| 274 | A2J_HC_W136F- | artificial | ATDMRPS |
| | LCDR2 | | |
| 275 | A2J_HC_W136F- | artificial | ALWYSNRWV |
| | LCDR3 | | |
| 276 | A2J_HC_W136F- | artificial | EVQLVESGGGLVQPGGSLKLSCAASGFTFNVYAMNWVRQAPGKGLEWVARIRSKYNNY |
| | VH | | ATYYADSVKKRFTISRDDSKNTAYLQMNNLKTEDTAVYYCVRHGNFGNSYLSWFAYWG |
| | | | QGTLVTVSS |
| 277 | A2J_HC_W136F- | artificial | QTVVTQEPSLTVSPGGTVTLTCRSSTGAVTSGYYPNWVQQKPGQAPRGLIGATDMRPS |
| | VL | | GTPARFSGSLLGGKAALTLSGVQPEDEAEYYCALWYSNRWVFGGGTKLTVL |
| 278 | A2J_HC_W136F_ | artificial | EVQLVESGGGLVQPGGSLKLSCAASGFTFNVYAMNWVRQAPGKGLEWVARIRSKYNNY |
| | LC_V44I- | | ATYYADSVKKRFTISRDDSKNTAYLQMNNLKTEDTAVYYCVRHGNFGNSYLSWFAYWG |
| | VH | | QGTLVTVSS |
| 279 | A2J_HC_W136F_ | artificial | QTVVTQEPSLTVSPGGTVTLTCRSSTGAVTSGYYPNWIQQKPGQAPRGLIGATDMRPS |
| | LC_V44I- | | GTPARFSGSLLGGKAALTLSGVQPEDEAEYYCALWYSNRWVFGGGTKLTVL |
| | VL | | |
| 280 | A2J_HC_W136F_ | artificial | EVQLVESGGGLVQPGGSLKLSCAASGFTFNVYAMNWVRQAPGKGLEWVARIRSKYNNY |
| | LC_V44L- | | ATYYADSVKKRFTISRDDSKNTAYLQMNNLKTEDTAVYYCVRHGNFGNSYLSWFAYWG |
| | VH | | QGTLVTVSS |
| 281 | A2J_HC_W136F_ | artificial | QTVVTQEPSLTVSPGGTVTLTCRSSTGAVTSGYYPNWLQQKPGQAPRGLIGATDMRPS |
| | LC_V44L- | | GTPARFSGSLLGGKAALTLSGVQPEDEAEYYCALWYSNRWVFGGGTKLTVL |
| | VL | | |
| 282 | A2J_I2E- | artificial | VYAIN |
| | HCDR1 | | |
| 283 | A2J_I2E- | artificial | RIRSKYNNYATYYADAVKK |
| | HCDR2 | | |
| 284 | A2J_I2E- | artificial | AGNFGSSYLSWWAY |
| | HCDR3 | | |
| 285 | A2J_I2E- | artificial | RSSTGAVTSGYYPN |
| | LCDR1 | | |
| 286 | A2J_I2E- | artificial | ATDMRPS |
| | LCDR2 | | |
| 287 | A2J_I2E- | artificial | ALWYSNRWV |
| | LCDR3 | | |
| 288 | A2J_I2E- | artificial | EVQLVESGGGLVQPGGSLKLSCAASGFTFNVYAINWVRQAPGKGLEWVARIRSKYNNY |
| | VH | | ATYYADAVKKRFTISRDDSKNTVYLQMNNLKTEDTAVYYCARAGNFGSSYLSWWAYWG |
| | | | QGTLVTVSS |
| 289 | A2J_I2E- | artificial | QTVVTQEPSLTVSPGGTVTITCRSSTGAVTSGYYPNWVQKKPGQAPRGLIGATDMRPS |
| | VL | | GTPARFSGSLSGGKAALTLSGVQPEDEAEYYCALWYSNRWVFGSGTKLTVL |
| 290 | A2J_I2K- | artificial | VYAMN |
| | HCDR1 | | |
| 291 | A2J_I2K- | artificial | RIRSKYNNYATYYAEAVKG |
| | HCDR2 | | |
| 292 | A2J_I2K- | artificial | NENIGTSYLSWWAY |
| | HCDR3 | | |
| 293 | A2J_I2K- | artificial | RSSTGAVTSGYYPN |
| | LCDR1 | | |
| 294 | A2J_I2K- | artificial | ATDMRPS |
| | LCDR2 | | |
| 295 | A2J_I2K- | artificial | ALWYSNRWV |
| | LCDR3 | | |
| 296 | A2J_I2K- | artificial | EVQLVESGGGLVQPGGSLKLSCAASGFTFNVYAMNWVRQAPGKGLEWVARIRSKYNNY |
| | VH | | ATYYAEAVKGRFTISRDDSKNTVYLQMNNLKTEDTAVYYCVRNENIGTSYLSWWAYWG |
| | | | QGTLVTVSS |
| 297 | A2J_I2K- | artificial | QTVVTQEPSLTVSPGGTVTITCRSSTGAVTSGYYPNWVQQKPGQAPRGLIGATDMRPS |
| | VL | | GTPARFSGSLSGGKAALTLSGVQPEDEAEYYCALWYSNRWVFGSGTKLTVL |
| 298 | A2J_I2L- | artificial | VYAMN |
| | HCDR1 | | |
| 299 | A2J_I2L- | artificial | RIRSKYNNYATYYADAVKK |
| | HCDR2 | | |
| 300 | A2J_I2L- | artificial | AGNFGSSYLSWFAY |
| | HCDR3 | | |
| 301 | A2J_I2L- | artificial | RSSTGAVTSGYYPN |
| | LCDR1 | | |
| 302 | A2J_I2L- | artificial | ATDMRPS |
| | LCDR2 | | |
| 303 | A2J_I2L- | artificial | ALYYSNRWV |
| | LCDR3 | | |
| 304 | A2J_I2L- | artificial | EVQLVESGGGLVQPGGSLKLSCAASGFTFNVYAMNWVRQAPGKGMEWVARIRSKYNNY |
| | VH | | ATYYADAVKKRFTISRDDSKNTLYLQMNNLKTEDTAVYYCVRAGNFGSSYLSWFAYWG |
| | | | QGTLVTVSS |
| 305 | A2J_I2L- | artificial | QTVVTQEPSLTVSPGGTVTITCRSSTGAVTSGYYPNWIQKKPGQAPRGLIGATDMRPS |
| | VL | | GTPARFSGSLEGGKAALTLSGVQPEDEAEYYCALYYSNRWVFGSGTKLTVL |
| 306 | A2J_I2M- | artificial | VYAMN |
| | HCDR1 | | |
| 307 | A2J_I2M- | artificial | RIRSKYNNYATYYADAVKK |
| | HCDR2 | | |
| 308 | A2J_I2M- | artificial | AGNFGTSYLSWWAY |
| | HCDR3 | | |
| 309 | A2J_I2M- | artificial | RSSTGAVTSGYYPN |
| | LCDR1 | | |
| 310 | A2J_I2M- | artificial | ATDMRPS |
| | LCDR2 | | |
| 311 | A2J_I2M- | artificial | ALWYSNRWV |
| | LCDR3 | | |
| 312 | A2J_I2M- | artificial | EVQLVESGGGLVQPGGSLKLSCAASGFTFNVYAMNWVRQAPGKGMEWVARIRSKYNNY |
| | VH | | ATYYADAVKKRFTISRDDSKNTLYLQMNNLKTEDTAVYYCVRAGNFGTSYLSWWAYWG |
| | | | QGTLVTVSS |
| 313 | A2J_I2M- | artificial | QTVVTQEPSLTVSPGGTVTITCRSSTGAVTSGYYPNWVQKKPGQAPRGLIGATDMRPS |
| | VL | | GTPARFSGSLSGGKAALTLSGVQPEDEAEYYCALWYSNRWVFGSGTKLTVL |
| 314 | A2J_I2M2- | artificial | VYAIN |
| | HCDR1 | | |
| 315 | A2J_I2M2- | artificial | RIRSKYNNYATYYADAVKK |
| | HCDR2 | | |
| 316 | A2J_I2M2- | artificial | NANFGTSYLSWFAY |
| | HCDR3 | | |
| 317 | A2J_I2M2- | artificial | RSSTGAVTSGYYPN |
| | LCDR1 | | |
| 318 | A2J_I2M2- | artificial | ATDMRPS |
| | LCDR2 | | |
| 319 | A2J_I2M2- | artificial | ALWYSNRWV |
| | LCDR3 | | |
| 320 | A2J_I2M2- | artificial | EVQLVESGGGLVQPGGSLKLSCAASGFTFNVYAINWVREAPGKGLEWVARIRSKYNNY |
| | VH | | ATYYADAVKKRFTISRDDSKNTAYLQMNNLKTEDTAVYYCVRNANFGTSYLSWFAYWG |
| | | | QGTLVTVSS |
| 321 | A2J_I2M2- | artificial | QTVVTQEPSLTVSPGGTVTLTCRSSTGAVTSGYYPNWVQKKPGQAPRGLIGATDMRPS |
| | VL | | GTPARFSGSLLGGKAALTLSGVQPEDEAEYYCALWYSNRWVFGSGTKLTVL |
| 322 | A2J_LC_G141S- | artificial | EVQLVESGGGLVQPGGSLKLSCAASGFTFNVYAMNWVRQAPGKGLEWVARIRSKYNNY |
| | VH | | ATYYADSVKKRFTISRDDSKNTAYLQMNNLKTEDTAVYYCVRHGNFGNSYLSWWAYWG |
| | | | QGTLVTVSS |
| 323 | A2J_LC_G141S- | artificial | QTVVTQEPSLTVSPGGTVTLTCRSSTGAVTSGYYPNWVQQKPGQAPRGLIGATDMRPS |
| | VL | | GTPARFSGSLLGGKAALTLSGVQPEDEAEYYCALWYSNRWVFGSGTKLTVL |
| 324 | A2J_LC_G141S_ | artificial | EVQLVESGGGLVQPGGSLKLSCAASGFTFNVYAMNWVRQAPGKGLEWVARIRSKYNNY |
| | L21I-VH | | ATYYADSVKKRFTISRDDSKNTAYLQMNNLKTEDTAVYYCVRHGNFGNSYLSWWAYWG |
| | | | QGTLVTVSS |
| 325 | A2J_LC_G141S_ | artificial | QTVVTQEPSLTVSPGGTVTITCRSSTGAVTSGYYPNWVQQKPGQAPRGLIGATDMRPS |
| | L21I-VL | | GTPARFSGSLLGGKAALTLSGVQPEDEAEYYCALWYSNRWVFGSGTKLTVL |
| 326 | A2J_LC_L83E | artificial | EVQLVESGGGLVQPGGSLKLSCAASGFTFNVYAMNWVRQAPGKGLEWVARIRSKYNNY |
| | -VH | | ATYYADSVKKRFTISRDDSKNTAYLQMNNLKTEDTAVYYCVRHGNFGNSYLSWWAYWG |
| | | | QGTLVTVSS |
| 327 | A2J_LC_L83E | artificial | QTVVTQEPSLTVSPGGTVTLTCRSSTGAVTSGYYPNWVQQKPGQAPRGLIGATDMRPS |
| | -VL | | GTPARFSGSLEGGKAALTLSGVQPEDEAEYYCALWYSNRWVFGGGTKLTVL |
| 328 | A2J_LC_L83S | artificial | EVQLVESGGGLVQPGGSLKLSCAASGFTFNVYAMNWVRQAPGKGLEWVARIRSKYNNY |
| | -VH | | ATYYADSVKKRFTISRDDSKNTAYLQMNNLKTEDTAVYYCVRHGNFGNSYLSWWAYWG |
| | | | QGTLVTVSS |
| 329 | A2J_LC_L83S | artificial | QTVVTQEPSLTVSPGGTVTLTCRSSTGAVTSGYYPNWVQQKPGQAPRGLIGATDMRPS |
| | -VL | | GTPARFSGSLSGGKAALTLSGVQPEDEAEYYCALWYSNRWVFGGGTKLTVL |
| 330 | A2J_LC_Q46E | artificial | EVQLVESGGGLVQPGGSLKLSCAASGFTFNVYAMNWVRQAPGKGLEWVARIRSKYNNY |
| | -VH | | ATYYADSVKKRFTISRDDSKNTAYLQMNNLKTEDTAVYYCVRHGNFGNSYLSWWAYWG |
| | | | QGTLVTVSS |
| 331 | A2J_LC_Q46E | artificial | QTVVTQEPSLTVSPGGTVTLTCRSSTGAVTSGYYPNWVQEKPGQAPRGLIGATDMRPS |
| | -VL | | GTPARFSGSLLGGKAALTLSGVQPEDEAEYYCALWYSNRWVFGGGTKLTVL |
| 332 | A2J_LC_Q46K | artificial | EVQLVESGGGLVQPGGSLKLSCAASGFTFNVYAMNWVRQAPGKGLEWVARIRSKYNNY |
| | -VH | | ATYYADSVKKRFTISRDDSKNTAYLQMNNLKTEDTAVYYCVRHGNFGNSYLSWWAYWG |
| | | | QGTLVTVSS |
| 333 | A2J_LC_Q46K | artificial | QTVVTQEPSLTVSPGGTVTLTCRSSTGAVTSGYYPNWVQKKPGQAPRGLIGATDMRPS |
| | -VL | | GTPARFSGSLLGGKAALTLSGVQPEDEAEYYCALWYSNRWVFGGGTKLTVL |
| 334 | A2J_LC_A107L- | artificial | VYAMN |
| | HCDR1 | | |
| 335 | A2J_LC_A107L- | artificial | RIRSKYNNYATYYADSVKK |
| | HCDR2 | | |
| 336 | A2J_LC_A107L- | artificial | HGNFGNSYLSWWAY |
| | HCDR3 | | |
| 337 | A2J_LC_A107L- | artificial | RSSTGAVTSGYYPN |
| | LCDR1 | | |
| 338 | A2J_LC_A107L- | artificial | ATDMRPS |
| | LCDR2 | | |
| 339 | A2J_LC_A107L- | artificial | LLWYSNRWV |
| | LCDR3 | | |
| 340 | A2J_LC_A107L- | artificial | EVQLVESGGGLVQPGGSLKLSCAASGFTFNVYAMNWVRQAPGKGLEWVARIRSKYNNY |
| | VH | | ATYYADSVKKRFTISRDDSKNTAYLQMNNLKTEDTAVYYCVRHGNFGNSYLSWWAYWG |
| | | | QGTLVTVSS |
| 341 | A2J_LC_A107L- | artificial | QTVVTQEPSLTVSPGGTVTLTCRSSTGAVTSGYYPNWVQQKPGQAPRGLIGATDMRPS |
| | VL | | GTPARFSGSLLGGKAALTLSGVQPEDEAEYYCLLWYSNRWVFGGGTKLTVL |
| 342 | A2J_LC_V44I | artificial | EVQLVESGGGLVQPGGSLKLSCAASGFTFNVYAMNWVRQAPGKGLEWVARIRSKYNNY |
| | -VH | | ATYYADSVKKRFTISRDDSKNTAYLQMNNLKTEDTAVYYCVRHGNFGNSYLSWWAYWG |
| | | | QGTLVTVSS |
| 343 | A2J_LC_V44I | artificial | QTVVTQEPSLTVSPGGTVTLTCRSSTGAVTSGYYPNWIQQKPGQAPRGLIGATDMRPS |
| | -VL | | GTPARFSGSLLGGKAALTLSGVQPEDEAEYYCALWYSNRWVFGGGTKLTVL |
| 344 | A2J_LC_V44L | artificial | EVQLVESGGGLVQPGGSLKLSCAASGFTFNVYAMNWVRQAPGKGLEWVARIRSKYNNY |
| | -VH | | ATYYADSVKKRFTISRDDSKNTAYLQMNNLKTEDTAVYYCVRHGNFGNSYLSWWAYWG |
| | | | QGTLVTVSS |
| 345 | A2J_LC_V44L | artificial | QTVVTQEPSLTVSPGGTVTLTCRSSTGAVTSGYYPNWLQQKPGQAPRGLIGATDMRPS |
| | -VL | | GTPARFSGSLLGGKAALTLSGVQPEDEAEYYCALWYSNRWVFGGGTKLTVL |
| 346 | A2J_LC_V44M | artificial | EVQLVESGGGLVQPGGSLKLSCAASGFTFNVYAMNWVRQAPGKGLEWVARIRSKYNNY |
| | -VH | | ATYYADSVKKRFTISRDDSKNTAYLQMNNLKTEDTAVYYCVRHGNFGNSYLSWWAYWG |
| | | | QGTLVTVSS |
| 347 | A2J_LC_V44M | artificial | QTVVTQEPSLTVSPGGTVTLTCRSSTGAVTSGYYPNWMQQKPGQAPRGLIGATDMRPS |
| | -VL | | GTPARFSGSLLGGKAALTLSGVQPEDEAEYYCALWYSNRWVFGGGTKLTVL |
| 348 | A2J_LC_W109Y- | artificial | VYAMN |
| | HCDR1 | | |
| 349 | A2J_LC_W109Y- | artificial | RIRSKYNNYATYYADSVKK |
| | HCDR2 | | |
| 350 | A2J_LC_W109Y- | artificial | HGNFGNSYLSWWAY |
| | HCDR3 | | |
| 351 | A2J_LC_W109Y- | artificial | RSSTGAVTSGYYPN |
| | LCDR1 | | |
| 352 | A2J_LC_W109Y- | artificial | ATDMRPS |
| | LCDR2 | | |
| 353 | A2J_LC_W109Y- | artificial | ALYYSNRWV |
| | LCDR3 | | |
| 354 | A2J_LC_W109Y- | artificial | EVQLVESGGGLVQPGGSLKLSCAASGFTFNVYAMNWVRQAPGKGLEWVARIRSKYNNY |
| | VH | | ATYYADSVKKRFTISRDDSKNTAYLQMNNLKTEDTAVYYCVRHGNFGNSYLSWWAYWG |
| | | | QGTLVTVSS |
| 355 | A2J_LC_W109Y- | artificial | QTVVTQEPSLTVSPGGTVTLTCRSSTGAVTSGYYPNWVQQKPGQAPRGLIGATDMRPS |
| | VL | | GTPARFSGSLLGGKAALTLSGVQPEDEAEYYCALYYSNRWVFGGGTKLTVL |
| 356 | E1L-HCDR1 | artificial | KYAMN |
| 357 | E1L-HCDR2 | artificial | RIRSKYNNYATYYADSVKS |
| 358 | E1L-HCDR3 | artificial | HGNFGNSYTSYYAY |
| 359 | E1L-LCDR1 | artificial | GSSTGAVTSGYYPN |
| 360 | E1L-LCDR2 | artificial | GTKFLAP |
| 361 | E1L-LCDR3 | artificial | ALWYSNRWV |
| 362 | E1L-VH | artificial | EVQLVESGGGLVQPGGSLKLSCAASGFTFNKYAMNWVRQAPGKGLEWVARIRSKYNNY |
| | | | ATYYADSVKSRFTISRDDSKNTAYLQMNNLKTEDTAVYYCVRHGNFGNSYTSYYAYWG |
| | | | QGTLVTVSS |
| 363 | E1L-VL | artificial | QTVVTQEPSLTVSPGGTVTLTCGSSTGAVTSGYYPNWVQQKPGQAPRGLIGGTKFLAP |
| | | | GTPARFSGSLLGGKAALTLSGVQPEDEAEYYCALWYSNRWVFGGGTKLTVL |
| 364 | E1L_HC_A89I | artificial | EVQLVESGGGLVQPGGSLKLSCAASGFTFNKYAMNWVRQAPGKGLEWVARIRSKYNNY |
| | -VH | | ATYYADSVKSRFTISRDDSKNTIYLQMNNLKTEDTAVYYCVRHGNFGNSYTSYYAYWG |
| | | | QGTLVTVSS |
| 365 | E1L_HC_A89I | artificial | QTVVTQEPSLTVSPGGTVTLTCGSSTGAVTSGYYPNWVQQKPGQAPRGLIGGTKFLAP |
| | -VL | | GTPARFSGSLLGGKAALTLSGVQPEDEAEYYCALWYSNRWVFGGGTKLTVL |
| 366 | E1L_HC_A89L | artificial | EVQLVESGGGLVQPGGSLKLSCAASGFTFNKYAMNWVRQAPGKGLEWVARIRSKYNNY |
| | -VH | | ATYYADSVKSRFTISRDDSKNTLYLQMNNLKTEDTAVYYCVRHGNFGNSYTSYYAYWG |
| | | | QGTLVTVSS |
| 367 | E1L_HC_A89L | artificial | QTVVTQEPSLTVSPGGTVTLTCGSSTGAVTSGYYPNWVQQKPGQAPRGLIGGTKFLAP |
| | -VL | | GTPARFSGSLLGGKAALTLSGVQPEDEAEYYCALWYSNRWVFGGGTKLTVL |
| 368 | E1L_HC_A89T | artificial | EVQLVESGGGLVQPGGSLKLSCAASGFTFNKYAMNWVRQAPGKGLEWVARIRSKYNNY |
| | -VH | | ATYYADSVKSRFTISRDDSKNTTYLQMNNLKTEDTAVYYCVRHGNFGNSYTSYYAYWG |
| | | | QGTLVTVSS |
| 369 | E1L_HC_A89T | artificial | QTVVTQEPSLTVSPGGTVTLTCGSSTGAVTSGYYPNWVQQKPGQAPRGLIGGTKFLAP |
| | -VL | | GTPARFSGSLLGGKAALTLSGVQPEDEAEYYCALWYSNRWVFGGGTKLTVL |
| 370 | E1L_HC_A89V | artificial | EVQLVESGGGLVQPGGSLKLSCAASGFTFNKYAMNWVRQAPGKGLEWVARIRSKYNNY |
| | -VH | | ATYYADSVKSRFTISRDDSKNTVYLQMNNLKTEDTAVYYCVRHGNFGNSYTSYYAYWG |
| | | | QGTLVTVSS |
| 371 | E1L_HC_A89V | artificial | QTVVTQEPSLTVSPGGTVTLTCGSSTGAVTSGYYPNWVQQKPGQAPRGLIGGTKFLAP |
| | -VL | | GTPARFSGSLLGGKAALTLSGVQPEDEAEYYCALWYSNRWVFGGGTKLTVL |
| 372 | E1L_HC_L52M | artificial | EVQLVESGGGLVQPGGSLKLSCAASGFTENKYAMNWVRQAPGKGMEWVARIRSKYNNY |
| | -VH | | ATYYADSVKSRFTISRDDSKNTAYLQMNNLKTEDTAVYYCVRHGNFGNSYTSYYAYWG |
| | | | QGTLVTVSS |
| 373 | E1L_HC_L52M | artificial | QTVVTQEPSLTVSPGGTVTLTCGSSTGAVTSGYYPNWVQQKPGQAPRGLIGGTKFLAP |
| | -VL | | GTPARFSGSLLGGKAALTLSGVQPEDEAEYYCALWYSNRWVFGGGTKLTVL |
| 374 | E1L_HC_L52M_ | artificial | EVQLVESGGGLVQPGGSLKLSCAASGFTFNKYAMNWVRQAPGKGMEWVARIRSKYNNY |
| | LC_L21I_ | | ATYYADSVKSRFTISRDDSKNTAYLQMNNLKTEDTAVYYCVRHGNFGNSYTSYYAYWG |
| | Q46K_G141S- | | QGTLVTVSS |
| | VH | | |
| 375 | E1L_HC_L52M_ | artificial | QTVVTQEPSLTVSPGGTVTITCGSSTGAVTSGYYPNWVQKKPGQAPRGLIGGTKFLAP |
| | LC_L21I_ | | GTPARFSGSLLGGKAALTLSGVQPEDEAEYYCALWYSNRWVFGSGTKLTVL |
| | Q46K_G141S- | | |
| | VL | | |
| 376 | E1L_HC_L52M_ | artificial | EVQLVESGGGLVQPGGSLKLSCAASGFTFNKYAMNWVRQAPGKGMEWVARIRSKYNNY |
| | LC_Q46K- | | ATYYADSVKSRFTISRDDSKNTAYLQMNNLKTEDTAVYYCVRHGNFGNSYTSYYAYWG |
| | VH | | QGTLVTVSS |
| 377 | E1L_HC_L52M_ | artificial | QTVVTQEPSLTVSPGGTVTLTCGSSTGAVTSGYYPNWVQKKPGQAPRGLIGGTKFLAP |
| | LC_Q46K- | | GTPARFSGSLLGGKAALTLSGVQPEDEAEYYCALWYSNRWVFGGGTKLTVL |
| | VL | | |
| 378 | E1L_HC_L52M_ | artificial | EVQLVESGGGLVQPGGSLKLSCAASGFTFNKYAMNWVRQAPGKGMEWVARIRSKYNNY |
| | LC_Q46K_ | | ATYYADSVKSRFTISRDDSKNTAYLQMNNLKTEDTAVYYCVRHGNFGNSYTSYYAYWG |
| | 4G11S-VH | | QGTLVTVSS |
| 379 | E1L_HC_L52M_ | artificial | QTVVTQEPSLTVSPGGTVTLTCGSSTGAVTSGYYPNWVQKKPGQAPRGLIGGTKFLAP |
| | LC_Q46K_ | | GTPARFSGSLLGGKAALTLSGVQPEDEAEYYCALWYSNRWVFGSGTKLTVL |
| | G141S-VL | | |
| 380 | E1L_HC_L52V | artificial | EVQLVESGGGLVQPGGSLKLSCAASGFTFNKYAMNWVRQAPGKGVEWVARIRSKYNNY |
| | -VH | | ATYYADSVKSRFTISRDDSKNTAYLQMNNLKTEDTAVYYCVRHGNFGNSYTSYYAYWG |
| | | | QGTLVTVSS |
| 381 | E1L_HC_L52V | artificial | QTVVTQEPSLTVSPGGTVTLTCGSSTGAVTSGYYPNWVQQKPGQAPRGLIGGTKFLAP |
| | -VL | | GTPARFSGSLLGGKAALTLSGVQPEDEAEYYCALWYSNRWVFGGGTKLTVL |
| 382 | E1L_HC_L52V_ | artificial | EVQLVESGGGLVQPGGSLKLSCAASGFTFNKYAMNWVRQAPGKGVEWVARIRSKYNNY |
| | LC_V44F- | | ATYYADSVKSRFTISRDDSKNTAYLQMNNLKTEDTAVYYCVRHGNFGNSYTSYYAYWG |
| | VH | | QGTLVTVSS |
| 383 | E1L_HC_L52V_ | artificial | QTVVTQEPSLTVSPGGTVTLTCGSSTGAVTSGYYPNWFQQKPGQAPRGLIGGTKFLAP |
| | LC_V44F- | | GTPARFSGSLLGGKAALTLSGVQPEDEAEYYCALWYSNRWVFGGGTKLTVL |
| | VL | | |
| 384 | E1L_HC_L52V_ | artificial | EVQLVESGGGLVQPGGSLKLSCAASGFTFNKYAMNWVRQAPGKGVEWVARIRSKYNNY |
| | LC_V44Y- | | ATYYADSVKSRFTISRDDSKNTAYLQMNNLKTEDTAVYYCVRHGNFGNSYTSYYAYWG |
| | VH | | QGTLVTVSS |
| 385 | E1L_HC_L52V_ | artificial | QTVVTQEPSLTVSPGGTVTLTCGSSTGAVTSGYYPNWYQQKPGQAPRGLIGGTKFLAP |
| | LC_V44Y- | | GTPARFSGSLLGGKAALTLSGVQPEDEAEYYCALWYSNRWVFGGGTKLTVL |
| | VL | | |
| 386 | E1L_HC_M41F_ | artificial | KYAFN |
| | A89V- | | |
| | HCDR1 | | |
| 387 | E1L_HC_M41F_ | artificial | RIRSKYNNYATYYADSVKS |
| | A89V-HCDR2 | | |
| 388 | E1L_HC_M41F_ | artificial | HGNFGNSYTSYYAY |
| | A89V- | | |
| | HCDR3 | | |
| 389 | E1L_HC_M41F_ | artificial | GSSTGAVTSGYYPN |
| | A89V- | | |
| | LCDR1 | | |
| 390 | E1L_HC_M41F_ | artificial | GTKFLAP |
| | A89V- | | |
| | LCDR2 | | |
| 391 | E1L_HC_M41F_ | artificial | ALWYSNRWV |
| | A89V-LCDR3 | | |
| 392 | E1L_HC_M41F_ | artificial | EVQLVESGGGLVQPGGSLKLSCAASGFTFNKYAFNWVRQAPGKGLEWVARIRSKYNNY |
| | A89V-VH | | ATYYADSVKSRFTISRDDSKNTVYLQMNNLKTEDTAVYYCVRHGNFGNSYTSYYAYWG |
| | | | QGTLVTVSS |
| 393 | E1L_HC_M41F_ | artificial | QTVVTQEPSLTVSPGGTVTLTCGSSTGAVTSGYYPNWVQQKPGQAPRGLIGGTKFLAP |
| | A89V-VL | | GTPARFSGSLLGGKAALTLSGVQPEDEAEYYCALWYSNRWVFGGGTKLTVL |
| 394 | E1L_HC_M41I | artificial | KYAIN |
| | -HCDR1 | | |
| 395 | E1L_HC_M41I | artificial | RIRSKYNNYATYYADSVKS |
| | -HCDR2 | | |
| 396 | E1L_HC_M41I | artificial | HGNFGNSYTSYYAY |
| | -HCDR3 | | |
| 397 | E1L_HC_M41I | artificial | GSSTGAVTSGYYPN |
| | -LCDR1 | | |
| 398 | E1L_HC_M41I | artificial | GTKFLAP |
| | -LCDR2 | | |
| 399 | E1L_HC_M41I | artificial | ALWYSNRWV |
| | -LCDR3 | | |
| 400 | E1L_HC_M41I | artificial | EVQLVESGGGLVQPGGSLKLSCAASGFTFNKYAINWVRQAPGKGLEWVARIRSKYNNY |
| | -VH | | ATYYADSVKSRFTISRDDSKNTAYLQMNNLKTEDTAVYYCVRHGNFGNSYTSYYAYWG |
| | | | QGTLVTVSS |
| 401 | E1L_HC_M41I | artificial | QTVVTQEPSLTVSPGGTVTLTCGSSTGAVTSGYYPNWVQQKPGQAPRGLIGGTKFLAP |
| | -VL | | GTPARFSGSLLGGKAALTLSGVQPEDEAEYYCALWYSNRWVFGGGTKLTVL |
| 402 | E1L_HC_M41I_ | artificial | EVQLVESGGGLVQPGGSLKLSCAASGFTFNKYAINWVRQAPGKGLEWVARIRSKYNNY |
| | A89L-VH | | ATYYADSVKSRFTISRDDSKNTLYLQMNNLKTEDTAVYYCVRHGNFGNSYTSYYAYWG |
| | | | QGTLVTVSS |
| 403 | E1L_HC_M41I_ | artificial | QTVVTQEPSLTVSPGGTVTLTCGSSTGAVTSGYYPNWVQQKPGQAPRGLIGGTKFLAP |
| | A89L-VL | | GTPARFSGSLLGGKAALTLSGVQPEDEAEYYCALWYSNRWVFGGGTKLTVL |
| 404 | E1L_HC_M41I_ | artificial | EVQLVESGGGLVQPGGSLKLSCAASGFTENKYAINWVRQAPGKGLEWVARIRSKYNNY |
| | A89V-VH | | ATYYADSVKSRFTISRDDSKNTVYLQMNNLKTEDTAVYYCVRHGNFGNSYTSYYAYWG |
| | | | QGTLVTVSS |
| 405 | E1L_HC_M41I_ | artificial | QTVVTQEPSLTVSPGGTVTLTCGSSTGAVTSGYYPNWVQQKPGQAPRGLIGGTKFLAP |
| | A89V-VL | | GTPARFSGSLLGGKAALTLSGVQPEDEAEYYCALWYSNRWVFGGGTKLTVL |
| 406 | E1L_HC_M41I_ | artificial | EVQLVESGGGLVQPGGSLKLSCAASGFTFNKYAINWVRQAPGKGLEWVARIRSKYNNY |
| | A89V_LC_ | | ATYYADSVKSRFTISRDDSKNTVYLQMNNLKTEDTAVYYCVRHGNFGNSYTSYYAYWG |
| | L21I_Q46K_ | | QGTLVTVSS |
| | G141S-VH | | |
| 407 | E1L_HC_M41I_ | artificial | QTVVTQEPSLTVSPGGTVTITCGSSTGAVTSGYYPNWVQKKPGQAPRGLIGGTKFLAP |
| | A89V_LC_ | | GTPARFSGSLLGGKAALTLSGVQPEDEAEYYCALWYSNRWVFGSGTKLTVL |
| | L21I_Q46K_ | | |
| | G141S-VL | | |
| 408 | E1L_HC_M41I_ | artificial | EVQLVESGGGLVQPGGSLKLSCAASGFTFNKYAINWVRQAPGKGLEWVARIRSKYNNY |
| | A89V_LC_ | | ATYYADSVKSRFTISRDDSKNTVYLQMNNLKTEDTAVYYCVRHGNFGNSYTSYYAYWG |
| | Q46K-VH | | QGTLVTVSS |
| 409 | E1L_HC_M41I_ | artificial | QTVVTQEPSLTVSPGGTVTLTCGSSTGAVTSGYYPNWVQKKPGQAPRGLIGGTKFLAP |
| | A89V_LC_ | | GTPARFSGSLLGGKAALTLSGVQPEDEAEYYCALWYSNRWVFGGGTKLTVL |
| | Q46K-VL | | |
| 410 | E1L_HC_M41I_ | artificial | EVQLVESGGGLVQPGGSLKLSCAASGFTENKYAINWVRQAPGKGVEWVARIRSKYNNY |
| | L52V_A89V_ | | ATYYADSVKSRFTISRDDSKNTVYLQMNNLKTEDTAVYYCVRHGNFGNSYTSYYAYWG |
| | LC_L21I_ | | QGTLVTVSS |
| | Q46K_G141S_ | | |
| | V44F-VH | | |
| 411 | E1L_HC_M41I_ | artificial | QTVVTQEPSLTVSPGGTVTITCGSSTGAVTSGYYPNWFQKKPGQAPRGLIGGTKFLAP |
| | L52V_A89V_ | | GTPARFSGSLLGGKAALTLSGVQPEDEAEYYCALWYSNRWVFGSGTKLTVL |
| | LC_L21I_ | | |
| | Q46K_G141S_ | | |
| | V44F-VL | | |
| 412 | E1L_HC_M41I_ | artificial | EVQLVESGGGLVQPGGSLKLSCAASGFTFNKYAINWVRQAPGKGLEWVARIRSKYNNY |
| | LC_L21I_ | | ATYYADSVKSRFTISRDDSKNTAYLQMNNLKTEDTAVYYCVRHGNFGNSYTSYYAYWG |
| | Q46K_G141S- | | QGTLVTVSS |
| | VH | | |
| 413 | E1L_HC_M41I_ | artificial | QTVVTQEPSLTVSPGGTVTITCGSSTGAVTSGYYPNWVQKKPGQAPRGLIGGTKFLAP |
| | LC_L21I_ | | GTPARFSGSLLGGKAALTLSGVQPEDEAEYYCALWYSNRWVFGSGTKLTVL |
| | Q46K_G141S- | | |
| | VL | | |
| 414 | E1L_HC_M41I_ | artificial | EVQLVESGGGLVQPGGSLKLSCAASGFTFNKYAINWVREAPGKGLEWVARIRSKYNNY |
| | Q46E_A89V_ | | ATYYADSVKSRFTISRDDSKNTVYLQMNNLKTEDTAVYYCVRHGNFGNSYTSYYAYWG |
| | LC_L21I_ | | QGTLVTVSS |
| | Q46K_G141S- | | |
| | VH | | |
| 415 | E1L_HC_M41I_ | artificial | QTVVTQEPSLTVSPGGTVTITCGSSTGAVTSGYYPNWVQKKPGQAPRGLIGGTKFLAP |
| | Q46E_A89V_ | | GTPARFSGSLLGGKAALTLSGVQPEDEAEYYCALWYSNRWVFGSGTKLTVL |
| | LC_L21I_ | | |
| | Q46K_G141S- | | |
| | VL | | |
| 416 | E1L_HC_M41V | artificial | KYAVN |
| | -HCDR1 | | |
| 417 | E1L_HC_M41V | artificial | RIRSKYNNYATYYADSVKS |
| | -HCDR2 | | |
| 418 | E1L_HC_M41V | artificial | HGNFGNSYTSYYAY |
| | -HCDR3 | | |
| 419 | E1L_HC_M41V | artificial | GSSTGAVTSGYYPN |
| | -LCDR1 | | |
| 420 | E1L_HC_M41V | artificial | GTKFLAP |
| | -LCDR2 | | |
| 421 | E1L_HC_M41V | artificial | ALWYSNRWV |
| | -LCDR3 | | |
| 422 | E1L_HC_M41V | artificial | EVQLVESGGGLVQPGGSLKLSCAASGFTFNKYAVNWVRQAPGKGLEWVARIRSKYNNY |
| | -VH | | ATYYADSVKSRFTISRDDSKNTAYLQMNNLKTEDTAVYYCVRHGNFGNSYTSYYAYWG |
| | | | QGTLVTVSS |
| 423 | E1L_HC_M41V | artificial | QTVVTQEPSLTVSPGGTVTLTCGSSTGAVTSGYYPNWVQQKPGQAPRGLIGGTKFLAP |
| | -VL | | GTPARFSGSLLGGKAALTLSGVQPEDEAEYYCALWYSNRWVFGGGTKLTVL |
| 424 | E1L_HC_Q46D | artificial | EVQLVESGGGLVQPGGSLKLSCAASGFTENKYAMNWVRDAPGKGLEWVARIRSKYNNY |
| | -VH | | ATYYADSVKSRFTISRDDSKNTAYLQMNNLKTEDTAVYYCVRHGNFGNSYTSYYAYWG |
| | | | QGTLVTVSS |
| 425 | E1L_HC_Q46D | artificial | QTVVTQEPSLTVSPGGTVTLTCGSSTGAVTSGYYPNWVQQKPGQAPRGLIGGTKFLAP |
| | -VL | | GTPARFSGSLLGGKAALTLSGVQPEDEAEYYCALWYSNRWVFGGGTKLTVL |
| 426 | E1L_HC_Q46D_ | artificial | EVQLVESGGGLVQPGGSLKLSCAASGFTFNKYAMNWVRDAPGKGLEWVARIRSKYNNY |
| | LC_L21I_ | | ATYYADSVKSRFTISRDDSKNTAYLQMNNLKTEDTAVYYCVRHGNFGNSYTSYYAYWG |
| | Q46K_G141S- | | QGTLVTVSS |
| | VH | | |
| 427 | E1L_HC_Q46D_ | artificial | QTVVTQEPSLTVSPGGTVTITCGSSTGAVTSGYYPNWVQKKPGQAPRGLIGGTKFLAP |
| | LC_L21I_ | | GTPARFSGSLLGGKAALTLSGVQPEDEAEYYCALWYSNRWVFGSGTKLTVL |
| | Q46K_G141S- | | |
| | VL | | |
| 428 | E1L_HC_Q46D_ | artificial | EVQLVESGGGLVQPGGSLKLSCAASGFTFNKYAMNWVRDAPGKGLEWVARIRSKYNNY |
| | LC_L21I_ | | ATYYADSVKSRFTISRDDSKNTAYLQMNNLKTEDTAVYYCVRHGNFGNSYTSYYAYWG |
| | Q46R_G141S- | | QGTLVTVSS |
| | VH | | |
| 429 | E1L_HC_Q46D_ | artificial | QTVVTQEPSLTVSPGGTVTITCGSSTGAVTSGYYPNWVQRKPGQAPRGLIGGTKFLAP |
| | LC_L21I_ | | GTPARFSGSLLGGKAALTLSGVQPEDEAEYYCALWYSNRWVFGSGTKLTVL |
| | Q46R_G141S- | | |
| | VL | | |
| 430 | E1L_HC_Q46D_ | artificial | EVQLVESGGGLVQPGGSLKLSCAASGFTFNKYAMNWVRDAPGKGLEWVARIRSKYNNY |
| | LC_Q46K- | | ATYYADSVKSRFTISRDDSKNTAYLQMNNLKTEDTAVYYCVRHGNFGNSYTSYYAYWG |
| | VH | | QGTLVTVSS |
| 431 | E1L_HC_Q46D_ | artificial | QTVVTQEPSLTVSPGGTVTLTCGSSTGAVTSGYYPNWVQKKPGQAPRGLIGGTKFLAP |
| | LC_Q46K- | | GTPARFSGSLLGGKAALTLSGVQPEDEAEYYCALWYSNRWVFGGGTKLTVL |
| | VL | | |
| 432 | E1L_HC_Q46D_ | artificial | EVQLVESGGGLVQPGGSLKLSCAASGFTFNKYAMNWVRDAPGKGLEWVARIRSKYNNY |
| | LC_Q46K_ | | ATYYADSVKSRFTISRDDSKNTAYLQMNNLKTEDTAVYYCVRHGNFGNSYTSYYAYWG |
| | G141S-VH | | QGTLVTVSS |
| 433 | E1L_HC_Q46D_ | artificial | QTVVTQEPSLTVSPGGTVTLTCGSSTGAVTSGYYPNWVQKKPGQAPRGLIGGTKFLAP |
| | LC_Q46K_ | | GTPARFSGSLLGGKAALTLSGVQPEDEAEYYCALWYSNRWVFGSGTKLTVL |
| | G141S-VL | | |
| 434 | E1L_HC_Q46D_ | artificial | EVQLVESGGGLVQPGGSLKLSCAASGFTFNKYAMNWVRDAPGKGLEWVARIRSKYNNY |
| | LC_Q46R- | | ATYYADSVKSRFTISRDDSKNTAYLQMNNLKTEDTAVYYCVRHGNFGNSYTSYYAYWG |
| | VH | | QGTLVTVSS |
| 435 | E1L_HC_Q46D_ | artificial | QTVVTQEPSLTVSPGGTVTLTCGSSTGAVTSGYYPNWVQRKPGQAPRGLIGGTKFLAP |
| | LC_Q46R- | | GTPARFSGSLLGGKAALTLSGVQPEDEAEYYCALWYSNRWVFGGGTKLTVL |
| | VL | | |
| 436 | E1L_HC_Q46D_ | artificial | EVQLVESGGGLVQPGGSLKLSCAASGFTFNKYAMNWVRDAPGKGLEWVARIRSKYNNY |
| | LC_Q46R_ | | ATYYADSVKSRFTISRDDSKNTAYLQMNNLKTEDTAVYYCVRHGNFGNSYTSYYAYWG |
| | G141S-VH | | QGTLVTVSS |
| 437 | E1L_HC_Q46D_ | artificial | QTVVTQEPSLTVSPGGTVTLTCGSSTGAVTSGYYPNWVQRKPGQAPRGLIGGTKFLAP |
| | LC_Q46R_ | | GTPARFSGSLLGGKAALTLSGVQPEDEAEYYCALWYSNRWVFGSGTKLTVL |
| | G141S-VL | | |
| 438 | E1L_HC_Q46E | artificial | EVQLVESGGGLVQPGGSLKLSCAASGFTFNKYAMNWVREAPGKGLEWVARIRSKYNNY |
| | -VH | | ATYYADSVKSRFTISRDDSKNTAYLQMNNLKTEDTAVYYCVRHGNFGNSYTSYYAYWG |
| | | | QGTLVTVSS |
| 439 | E1L_HC_Q46E | artificial | QTVVTQEPSLTVSPGGTVTLTCGSSTGAVTSGYYPNWVQQKPGQAPRGLIGGTKFLAP |
| | -VL | | GTPARFSGSLLGGKAALTLSGVQPEDEAEYYCALWYSNRWVFGGGTKLTVL |
| 440 | E1L_HC_Q46E_ | artificial | EVQLVESGGGLVQPGGSLKLSCAASGFTFNKYAMNWVREAPGKGLEWVARIRSKYNNY |
| | LC_L21I_ | | ATYYADSVKSRFTISRDDSKNTAYLQMNNLKTEDTAVYYCVRHGNFGNSYTSYYAYWG |
| | Q46K_G141S- | | QGTLVTVSS |
| | VH | | |
| 441 | E1L_HC_Q46E_ | artificial | QTVVTQEPSLTVSPGGTVTITCGSSTGAVTSGYYPNWVQKKPGQAPRGLIGGTKFLAP |
| | LC_L21I_ | | GTPARFSGSLLGGKAALTLSGVQPEDEAEYYCALWYSNRWVFGSGTKLTVL |
| | Q46K_G141S- | | |
| | VL | | |
| 442 | E1L_HC_Q46E_ | artificial | EVQLVESGGGLVQPGGSLKLSCAASGFTFNKYAMNWVREAPGKGLEWVARIRSKYNNY |
| | LC_L21I_ | | ATYYADSVKSRFTISRDDSKNTAYLQMNNLKTEDTAVYYCVRHGNFGNSYTSYYAYWG |
| | Q46R_G141S- | | QGTLVTVSS |
| | VH | | |
| 443 | E1L_HC_Q46E_ | artificial | QTVVTQEPSLTVSPGGTVTITCGSSTGAVTSGYYPNWVQRKPGQAPRGLIGGTKFLAP |
| | LC_L21I_ | | GTPARFSGSLLGGKAALTLSGVQPEDEAEYYCALWYSNRWVFGSGTKLTVL |
| | Q46R_G141S- | | |
| | VL | | |
| 444 | E1L_HC_Q46E_ | artificial | EVQLVESGGGLVQPGGSLKLSCAASGFTFNKYAMNWVREAPGKGLEWVARIRSKYNNY |
| | LC_Q46K- | | ATYYADSVKSRFTISRDDSKNTAYLQMNNLKTEDTAVYYCVRHGNFGNSYTSYYAYWG |
| | VH | | QGTLVTVSS |
| 445 | E1L_HC_Q46E_ | artificial | QTVVTQEPSLTVSPGGTVTLTCGSSTGAVTSGYYPNWVQKKPGQAPRGLIGGTKFLAP |
| | LC_Q46K- | | GTPARFSGSLLGGKAALTLSGVQPEDEAEYYCALWYSNRWVFGGGTKLTVL |
| | VL | | |
| 446 | E1L_HC_Q46E_ | artificial | EVQLVESGGGLVQPGGSLKLSCAASGFTFNKYAMNWVREAPGKGLEWVARIRSKYNNY |
| | LC_Q46K_ | | ATYYADSVKSRFTISRDDSKNTAYLQMNNLKTEDTAVYYCVRHGNFGNSYTSYYAYWG |
| | G141S-VH | | QGTLVTVSS |
| 447 | E1L_HC_Q46E_ | artificial | QTVVTQEPSLTVSPGGTVTLTCGSSTGAVTSGYYPNWVQKKPGQAPRGLIGGTKFLAP |
| | LC_Q46K_ | | GTPARFSGSLLGGKAALTLSGVQPEDEAEYYCALWYSNRWVFGSGTKLTVL |
| | G141S-VL | | |
| 448 | E1L_HC_Q46E_ | artificial | EVQLVESGGGLVQPGGSLKLSCAASGFTFNKYAMNWVREAPGKGLEWVARIRSKYNNY |
| | LC_Q46R- | | ATYYADSVKSRFTISRDDSKNTAYLQMNNLKTEDTAVYYCVRHGNFGNSYTSYYAYWG |
| | VH | | QGTLVTVSS |
| 449 | E1L_HC_Q46E_ | artificial | QTVVTQEPSLTVSPGGTVTLTCGSSTGAVTSGYYPNWVQRKPGQAPRGLIGGTKFLAP |
| | LC_Q46R- | | GTPARFSGSLLGGKAALTLSGVQPEDEAEYYCALWYSNRWVFGGGTKLTVL |
| | VL | | |
| 450 | E1L_HC_Q46E_ | artificial | EVQLVESGGGLVQPGGSLKLSCAASGFTFNKYAMNWVREAPGKGLEWVARIRSKYNNY |
| | LC_Q46R_ | | ATYYADSVKSRFTISRDDSKNTAYLQMNNLKTEDTAVYYCVRHGNFGNSYTSYYAYWG |
| | G141S-VH | | QGTLVTVSS |
| 451 | E1L_HC_Q46E_ | artificial | QTVVTQEPSLTVSPGGTVTLTCGSSTGAVTSGYYPNWVQRKPGQAPRGLIGGTKFLAP |
| | LC_Q46R_ | | GTPARFSGSLLGGKAALTLSGVQPEDEAEYYCALWYSNRWVFGSGTKLTVL |
| | G141S-VL | | |
| 452 | E1L_HC_Q46K | artificial | EVQLVESGGGLVQPGGSLKLSCAASGFTENKYAMNWVRKAPGKGLEWVARIRSKYNNY |
| | -VH | | ATYYADSVKSRFTISRDDSKNTAYLQMNNLKTEDTAVYYCVRHGNFGNSYTSYYAYWG |
| | | | QGTLVTVSS |
| 453 | E1L_HC_Q46K | artificial | QTVVTQEPSLTVSPGGTVTLTCGSSTGAVTSGYYPNWVQQKPGQAPRGLIGGTKFLAP |
| | -VL | | GTPARFSGSLLGGKAALTLSGVQPEDEAEYYCALWYSNRWVFGGGTKLTVL |
| 454 | E1L_HC_Q46R | artificial | EVQLVESGGGLVQPGGSLKLSCAASGFTFNKYAMNWVRRAPGKGLEWVARIRSKYNNY |
| | -VH | | ATYYADSVKSRFTISRDDSKNTAYLQMNNLKTEDTAVYYCVRHGNFGNSYTSYYAYWG |
| | | | QGTLVTVSS |
| 455 | E1L_HC_Q46R | artificial | QTVVTQEPSLTVSPGGTVTLTCGSSTGAVTSGYYPNWVQQKPGQAPRGLIGGTKFLAP |
| | -VL | | GTPARFSGSLLGGKAALTLSGVQPEDEAEYYCALWYSNRWVFGGGTKLTVL |
| 456 | E1L_HC_V107A- | artificial | EVQLVESGGGLVQPGGSLKLSCAASGFTFNKYAMNWVRQAPGKGLEWVARIRSKYNNY |
| | VH | | ATYYADSVKSRFTISRDDSKNTAYLQMNNLKTEDTAVYYCARHGNFGNSYTSYYAYWG |
| | | | QGTLVTVSS |
| 457 | E1L_HC_V107A- | artificial | QTVVTQEPSLTVSPGGTVTLTCGSSTGAVTSGYYPNWVQQKPGQAPRGLIGGTKFLAP |
| | VL | | GTPARFSGSLLGGKAALTLSGVQPEDEAEYYCALWYSNRWVFGGGTKLTVL |
| 458 | E1L_HC_V107L- | artificial | EVQLVESGGGLVQPGGSLKLSCAASGFTFNKYAMNWVRQAPGKGLEWVARIRSKYNNY |
| | VH | | ATYYADSVKSRFTISRDDSKNTAYLQMNNLKTEDTAVYYCLRHGNFGNSYTSYYAYWG |
| | | | QGTLVTVSS |
| 459 | E1L_HC_V107L- | artificial | QTVVTQEPSLTVSPGGTVTLTCGSSTGAVTSGYYPNWVQQKPGQAPRGLIGGTKFLAP |
| | VL | | GTPARFSGSLLGGKAALTLSGVQPEDEAEYYCALWYSNRWVFGGGTKLTVL |
| 460 | E1L_HC_Y136F- | artificial | KYAMN |
| | HCDR1 | | |
| 461 | E1L_HC_Y136F- | artificial | RIRSKYNNYATYYADSVKS |
| | HCDR2 | | |
| 462 | E1L_HC_Y136F- | artificial | HGNFGNSYTSYFAY |
| | HCDR3 | | |
| 463 | E1L_HC_Y136F- | artificial | GSSTGAVTSGYYPN |
| | LCDR1 | | |
| 464 | E1L_HC_Y136F- | artificial | GTKFLAP |
| | LCDR2 | | |
| 465 | E1L_HC_Y136F- | artificial | ALWYSNRWV |
| | LCDR3 | | |
| 466 | E1L_HC_Y136F- | artificial | EVQLVESGGGLVQPGGSLKLSCAASGFTFNKYAMNWVRQAPGKGLEWVARIRSKYNNY |
| | VH | | ATYYADSVKSRFTISRDDSKNTAYLQMNNLKTEDTAVYYCVRHGNFGNSYTSYFAYWG |
| | | | QGTLVTVSS |
| 467 | E1L_HC_Y136F- | artificial | QTVVTQEPSLTVSPGGTVTLTCGSSTGAVTSGYYPNWVQQKPGQAPRGLIGGTKFLAP |
| | VL | | GTPARFSGSLLGGKAALTLSGVQPEDEAEYYCALWYSNRWVFGGGTKLTVL |
| 468 | E1L_HC_Y136F_ | artificial | EVQLVESGGGLVQPGGSLKLSCAASGFTFNKYAMNWVRQAPGKGLEWVARIRSKYNNY |
| | LC_V44I- | | ATYYADSVKSRFTISRDDSKNTAYLQMNNLKTEDTAVYYCVRHGNFGNSYTSYFAYWG |
| | VH | | QGTLVTVSS |
| 469 | E1L_HC_Y136F_ | artificial | QTVVTQEPSLTVSPGGTVTLTCGSSTGAVTSGYYPNWIQQKPGQAPRGLIGGTKFLAP |
| | LC_V44I- | | GTPARFSGSLLGGKAALTLSGVQPEDEAEYYCALWYSNRWVFGGGTKLTVL |
| | VL | | |
| 470 | E1L_HC_Y136F_ | artificial | EVQLVESGGGLVQPGGSLKLSCAASGFTFNKYAMNWVRQAPGKGLEWVARIRSKYNNY |
| | LC_V44L- | | ATYYADSVKSRFTISRDDSKNTAYLQMNNLKTEDTAVYYCVRHGNFGNSYTSYFAYWG |
| | VH | | QGTLVTVSS |
| 471 | E1L_HC_Y136F_ | artificial | QTVVTQEPSLTVSPGGTVTLTCGSSTGAVTSGYYPNWLQQKPGQAPRGLIGGTKFLAP |
| | LC_V44L- | | GTPARFSGSLLGGKAALTLSGVQPEDEAEYYCALWYSNRWVFGGGTKLTVL |
| | VL | | |
| 472 | E1L_I2E- | artificial | KYAIN |
| | HCDR1 | | |
| 473 | E1L_I2E- | artificial | RIRSKYNNYATYYADAVKS |
| | HCDR2 | | |
| 474 | E1L_I2E- | artificial | AGNFGSSYTSYYAY |
| | HCDR3 | | |
| 475 | E1L_I2E- | artificial | GSSTGAVTSGYYPN |
| | LCDR1 | | |
| 476 | E1L_I2E- | artificial | GTKFLAP |
| | LCDR2 | | |
| 477 | E1L_I2E- | artificial | ALWYSNRWV |
| | LCDR3 | | |
| 478 | E1L_I2E- | artificial | EVQLVESGGGLVQPGGSLKLSCAASGFTFNKYAINWVRQAPGKGLEWVARIRSKYNNY |
| | VH | | ATYYADAVKSRFTISRDDSKNTVYLQMNNLKTEDTAVYYCARAGNFGSSYTSYYAYWG |
| | | | QGTLVTVSS |
| 479 | E1L_I2E- | artificial | QTVVTQEPSLTVSPGGTVTITCGSSTGAVTSGYYPNWVQKKPGQAPRGLIGGTKFLAP |
| | VL | | GTPARFSGSLSGGKAALTLSGVQPEDEAEYYCALWYSNRWVFGSGTKLTVL |
| 480 | E1L_I2K- | artificial | KYAMN |
| | HCDR1 | | |
| 481 | E1L_I2K- | artificial | RIRSKYNNYATYYAEAVKG |
| | HCDR2 | | |
| 482 | E1L_I2K- | artificial | NENIGTSYTSYYAY |
| | HCDR3 | | |
| 483 | E1L_I2K- | artificial | GSSTGAVTSGYYPN |
| | LCDR1 | | |
| 484 | E1L_I2K- | artificial | GTKFLAP |
| | LCDR2 | | |
| 485 | E1L_I2K- | artificial | ALWYSNRWV |
| | LCDR3 | | |
| 486 | E1L_I2K- | artificial | EVQLVESGGGLVQPGGSLKLSCAASGFTFNKYAMNWVRQAPGKGLEWVARIRSKYNNY |
| | VH | | ATYYAEAVKGRFTISRDDSKNTVYLQMNNLKTEDTAVYYCVRNENIGTSYTSYYAYWG |
| | | | QGTLVTVSS |
| 487 | E1L_I2K- | artificial | QTVVTQEPSLTVSPGGTVTITCGSSTGAVTSGYYPNWVQQKPGQAPRGLIGGTKFLAP |
| | VL | | GTPARFSGSLSGGKAALTLSGVQPEDEAEYYCALWYSNRWVFGSGTKLTVL |
| 488 | E1L_I2L- | artificial | KYAMN |
| | HCDR1 | | |
| 489 | E1L_I2L- | artificial | RIRSKYNNYATYYADAVKS |
| | HCDR2 | | |
| 490 | E1L_I2L- | artificial | AGNFGSSYTSYFAY |
| | HCDR3 | | |
| 491 | E1L_I2L- | artificial | GSSTGAVTSGYYPN |
| | LCDR1 | | |
| 492 | E1L_I2L- | artificial | GTKFLAP |
| | LCDR2 | | |
| 493 | E1L_I2L- | artificial | ALYYSNRWV |
| | LCDR3 | | |
| 494 | E1L_I2L- | artificial | EVQLVESGGGLVQPGGSLKLSCAASGFTENKYAMNWVRQAPGKGMEWVARIRSKYNNY |
| | VH | | ATYYADAVKSRFTISRDDSKNTLYLQMNNLKTEDTAVYYCVRAGNFGSSYTSYFAYWG |
| | | | QGTLVTVSS |
| 495 | E1L_I2L- | artificial | QTVVTQEPSLTVSPGGTVTITCGSSTGAVTSGYYPNWIQKKPGQAPRGLIGGTKFLAP |
| | VL | | GTPARFSGSLEGGKAALTLSGVQPEDEAEYYCALYYSNRWVFGSGTKLTVL |
| 496 | E1L_I2M- | artificial | KYAMN |
| | HCDR1 | | |
| 497 | E1L_I2M- | artificial | RIRSKYNNYATYYADAVKS |
| | HCDR2 | | |
| 498 | E1L_I2M- | artificial | AGNFGTSYTSYYAY |
| | HCDR3 | | |
| 499 | E1L_I2M- | artificial | GSSTGAVTSGYYPN |
| | LCDR1 | | |
| 500 | E1L_I2M- | artificial | GTKFLAP |
| | LCDR2 | | |
| 501 | E1L_I2M- | artificial | ALWYSNRWV |
| | LCDR3 | | |
| 502 | E1L_I2M- | artificial | EVQLVESGGGLVQPGGSLKLSCAASGFTFNKYAMNWVRQAPGKGMEWVARIRSKYNNY |
| | VH | | ATYYADAVKSRFTISRDDSKNTLYLQMNNLKTEDTAVYYCVRAGNFGTSYTSYYAYWG |
| | | | QGTLVTVSS |
| 503 | E1L_I2M- | artificial | QTVVTQEPSLTVSPGGTVTITCGSSTGAVTSGYYPNWVQKKPGQAPRGLIGGTKFLAP |
| | VL | | GTPARFSGSLSGGKAALTLSGVQPEDEAEYYCALWYSNRWVFGSGTKLTVL |
| 504 | E1L_I2M2- | artificial | KYAIN |
| | HCDR1 | | |
| 505 | E1L_I2M2- | artificial | RIRSKYNNYATYYADAVKS |
| | HCDR2 | | |
| 506 | E1L_I2M2- | artificial | NANFGTSYTSYFAY |
| | HCDR3 | | |
| 507 | E1L_I2M2- | artificial | GSSTGAVTSGYYPN |
| | LCDR1 | | |
| 508 | E1L_I2M2- | artificial | GTKFLAP |
| | LCDR2 | | |
| 509 | E1L_I2M2- | artificial | ALWYSNRWV |
| | LCDR3 | | |
| 510 | E1L_I2M2- | artificial | EVQLVESGGGLVQPGGSLKLSCAASGFTENKYAINWVREAPGKGLEWVARIRSKYNNY |
| | VH | | ATYYADAVKSRFTISRDDSKNTAYLQMNNLKTEDTAVYYCVRNANFGTSYTSYFAYWG |
| | | | QGTLVTVSS |
| 511 | E1L_I2M2- | artificial | QTVVTQEPSLTVSPGGTVTLTCGSSTGAVTSGYYPNWVQKKPGQAPRGLIGGTKFLAP |
| | VL | | GTPARFSGSLLGGKAALTLSGVQPEDEAEYYCALWYSNRWVFGSGTKLTVL |
| 512 | E1L_LC_G141S- | artificial | EVQLVESGGGLVQPGGSLKLSCAASGFTFNKYAMNWVRQAPGKGLEWVARIRSKYNNY |
| | VH | | ATYYADSVKSRFTISRDDSKNTAYLQMNNLKTEDTAVYYCVRHGNFGNSYTSYYAYWG |
| | | | QGTLVTVSS |
| 513 | E1L_LC_G141S- | artificial | QTVVTQEPSLTVSPGGTVTLTCGSSTGAVTSGYYPNWVQQKPGQAPRGLIGGTKFLAP |
| | VL | | GTPARFSGSLLGGKAALTLSGVQPEDEAEYYCALWYSNRWVFGSGTKLTVL |
| 514 | E1L_LC_G141S_ | artificial | EVQLVESGGGLVQPGGSLKLSCAASGFTFNKYAMNWVRQAPGKGLEWVARIRSKYNNY |
| | L21I-VH | | ATYYADSVKSRFTISRDDSKNTAYLQMNNLKTEDTAVYYCVRHGNFGNSYTSYYAYWG |
| | | | QGTLVTVSS |
| 515 | E1L_LC_G141S_ | artificial | QTVVTQEPSLTVSPGGTVTITCGSSTGAVTSGYYPNWVQQKPGQAPRGLIGGTKFLAP |
| | L21I-VL | | GTPARFSGSLLGGKAALTLSGVQPEDEAEYYCALWYSNRWVFGSGTKLTVL |
| 516 | E1L_LC_L83E | artificial | EVQLVESGGGLVQPGGSLKLSCAASGFTFNKYAMNWVRQAPGKGLEWVARIRSKYNNY |
| | -VH | | ATYYADSVKSRFTISRDDSKNTAYLQMNNLKTEDTAVYYCVRHGNFGNSYTSYYAYWG |
| | | | QGTLVTVSS |
| 517 | E1L_LC_L83E | artificial | QTVVTQEPSLTVSPGGTVTLTCGSSTGAVTSGYYPNWVQQKPGQAPRGLIGGTKFLAP |
| | -VL | | GTPARFSGSLEGGKAALTLSGVQPEDEAEYYCALWYSNRWVFGGGTKLTVL |
| 518 | E1L_LC_L83S | artificial | EVQLVESGGGLVQPGGSLKLSCAASGFTFNKYAMNWVRQAPGKGLEWVARIRSKYNNY |
| | -VH | | ATYYADSVKSRFTISRDDSKNTAYLQMNNLKTEDTAVYYCVRHGNFGNSYTSYYAYWG |
| | | | QGTLVTVSS |
| 519 | E1L_LC_L83S | artificial | QTVVTQEPSLTVSPGGTVTLTCGSSTGAVTSGYYPNWVQQKPGQAPRGLIGGTKFLAP |
| | -VL | | GTPARFSGSLSGGKAALTLSGVQPEDEAEYYCALWYSNRWVFGGGTKLTVL |
| 520 | E1L_LC_Q46E | artificial | EVQLVESGGGLVQPGGSLKLSCAASGFTFNKYAMNWVRQAPGKGLEWVARIRSKYNNY |
| | -VH | | ATYYADSVKSRFTISRDDSKNTAYLQMNNLKTEDTAVYYCVRHGNFGNSYTSYYAYWG |
| | | | QGTLVTVSS |
| 521 | E1L_LC_Q46E | artificial | QTVVTQEPSLTVSPGGTVTLTCGSSTGAVTSGYYPNWVQEKPGQAPRGLIGGTKFLAP |
| | -VL | | GTPARFSGSLLGGKAALTLSGVQPEDEAEYYCALWYSNRWVFGGGTKLTVL |
| 522 | E1L_LC_Q46K | artificial | EVQLVESGGGLVQPGGSLKLSCAASGFTFNKYAMNWVRQAPGKGLEWVARIRSKYNNY |
| | -VH | | ATYYADSVKSRFTISRDDSKNTAYLQMNNLKTEDTAVYYCVRHGNFGNSYTSYYAYWG |
| | | | QGTLVTVSS |
| 523 | E1L_LC_Q46K | artificial | QTVVTQEPSLTVSPGGTVTLTCGSSTGAVTSGYYPNWVQKKPGQAPRGLIGGTKFLAP |
| | -VL | | GTPARFSGSLLGGKAALTLSGVQPEDEAEYYCALWYSNRWVFGGGTKLTVL |
| 524 | E1L_LC_A107L- | artificial | KYAMN |
| | HCDR1 | | |
| 525 | E1L_LC_A107L- | artificial | RIRSKYNNYATYYADSVKS |
| | HCDR2 | | |
| 526 | E1L_LC_A107L- | artificial | HGNFGNSYTSYYAY |
| | HCDR3 | | |
| 527 | E1L_LC_A107L- | artificial | GSSTGAVTSGYYPN |
| | LCDR1 | | |
| 528 | E1L_LC_A107L- | artificial | GTKFLAP |
| | LCDR2 | | |
| 529 | E1L_LC_A107L- | artificial | LLWYSNRWV |
| | LCDR3 | | |
| 530 | E1L_LC_A107L- | artificial | EVQLVESGGGLVQPGGSLKLSCAASGFTFNKYAMNWVRQAPGKGLEWVARIRSKYNNY |
| | VH | | ATYYADSVKSRFTISRDDSKNTAYLQMNNLKTEDTAVYYCVRHGNFGNSYTSYYAYWG |
| | | | QGTLVTVSS |
| 531 | E1L_LC_A107L- | artificial | QTVVTQEPSLTVSPGGTVTLTCGSSTGAVTSGYYPNWVQQKPGQAPRGLIGGTKFLAP |
| | VL | | GTPARFSGSLLGGKAALTLSGVQPEDEAEYYCLLWYSNRWVFGGGTKLTVL |
| 532 | E1L_LC_V44I | artificial | EVQLVESGGGLVQPGGSLKLSCAASGFTFNKYAMNWVRQAPGKGLEWVARIRSKYNNY |
| | -VH | | ATYYADSVKSRFTISRDDSKNTAYLQMNNLKTEDTAVYYCVRHGNFGNSYTSYYAYWG |
| | | | QGTLVTVSS |
| 533 | E1L_LC_V44I | artificial | QTVVTQEPSLTVSPGGTVTLTCGSSTGAVTSGYYPNWIQQKPGQAPRGLIGGTKFLAP |
| | -VL | | GTPARFSGSLLGGKAALTLSGVQPEDEAEYYCALWYSNRWVFGGGTKLTVL |
| 534 | E1L_LC_V44L | artificial | EVQLVESGGGLVQPGGSLKLSCAASGFTFNKYAMNWVRQAPGKGLEWVARIRSKYNNY |
| | -VH | | ATYYADSVKSRFTISRDDSKNTAYLQMNNLKTEDTAVYYCVRHGNFGNSYTSYYAYWG |
| | | | QGTLVTVSS |
| 535 | E1L_LC_V44L | artificial | QTVVTQEPSLTVSPGGTVTLTCGSSTGAVTSGYYPNWLQQKPGQAPRGLIGGTKFLAP |
| | -VL | | GTPARFSGSLLGGKAALTLSGVQPEDEAEYYCALWYSNRWVFGGGTKLTVL |
| 536 | E1L_LC_V44M | artificial | EVQLVESGGGLVQPGGSLKLSCAASGFTFNKYAMNWVRQAPGKGLEWVARIRSKYNNY |
| | -VH | | ATYYADSVKSRFTISRDDSKNTAYLQMNNLKTEDTAVYYCVRHGNFGNSYTSYYAYWG |
| | | | QGTLVTVSS |
| 537 | E1L_LC_V44M | artificial | QTVVTQEPSLTVSPGGTVTLTCGSSTGAVTSGYYPNWMQQKPGQAPRGLIGGTKFLAP |
| | -VL | | GTPARFSGSLLGGKAALTLSGVQPEDEAEYYCALWYSNRWVFGGGTKLTVL |
| 538 | E1L_LC_W109Y- | artificial | KYAMN |
| | HCDR1 | | |
| 539 | E1L_LC_W109Y- | artificial | RIRSKYNNYATYYADSVKS |
| | HCDR2 | | |
| 540 | E1L_LC_W109Y- | artificial | HGNFGNSYTSYYAY |
| | HCDR3 | | |
| 541 | E1L_LC_W109Y- | artificial | GSSTGAVTSGYYPN |
| | LCDR1 | | |
| 542 | E1L_LC_W109Y- | artificial | GTKFLAP |
| | LCDR2 | | |
| 543 | E1L_LC_W109Y- | artificial | ALYYSNRWV |
| | LCDR3 | | |
| 544 | E1L_LC_W109Y- | artificial | EVQLVESGGGLVQPGGSLKLSCAASGFTFNKYAMNWVRQAPGKGLEWVARIRSKYNNY |
| | VH | | ATYYADSVKSRFTISRDDSKNTAYLQMNNLKTEDTAVYYCVRHGNFGNSYTSYYAYWG |
| | | | QGTLVTVSS |
| 545 | E1L_LC_W109Y- | artificial | QTVVTQEPSLTVSPGGTVTLTCGSSTGAVTSGYYPNWVQQKPGQAPRGLIGGTKFLAP |
| | VL | | GTPARFSGSLLGGKAALTLSGVQPEDEAEYYCALYYSNRWVFGGGTKLTVL |
| 546 | E2M-HCDR1 | artificial | GYAMN |
| 547 | E2M-HCDR2 | artificial | RIRSKYNNYATYYADSVKE |
| 548 | E2M-HCDR3 | artificial | HRNFGNSYLSWFAY |
| 549 | E2M-LCDR1 | artificial | RSSTGAVTSGYYPN |
| 550 | E2M-LCDR2 | artificial | ATDMRPS |
| 551 | E2M-LCDR3 | artificial | ALWYSNRWV |
| 552 | E2M-VH | artificial | EVQLVESGGGLVQPGGSLKLSCAASGFTFNGYAMNWVRQAPGKGLEWVARIRSKYNNY |
| | | | ATYYADSVKERFTISRDDSKNTAYLQMNNLKTEDTAVYYCVRHRNFGNSYLSWFAYWG |
| | | | QGTLVTVSS |
| 553 | E2M-VL | artificial | QTVVTQEPSLTVSPGGTVTLTCRSSTGAVTSGYYPNWVQQKPGQAPRGLIGATDMRPS |
| | | | GTPARFSGSLLGGKAALTLSGVQPEDEAEYYCALWYSNRWVFGGGTKLTVL |
| 554 | E2M_HC_A89I | artificial | EVQLVESGGGLVQPGGSLKLSCAASGFTFNGYAMNWVRQAPGKGLEWVARIRSKYNNY |
| | -VH | | ATYYADSVKERFTISRDDSKNTIYLQMNNLKTEDTAVYYCVRHRNFGNSYLSWFAYWG |
| | | | QGTLVTVSS |
| 555 | E2M_HC_A89I | artificial | QTVVTQEPSLTVSPGGTVTLTCRSSTGAVTSGYYPNWVQQKPGQAPRGLIGATDMRPS |
| | -VL | | GTPARFSGSLLGGKAALTLSGVQPEDEAEYYCALWYSNRWVFGGGTKLTVL |
| 556 | E2M_HC_A89L | artificial | EVQLVESGGGLVQPGGSLKLSCAASGFTFNGYAMNWVRQAPGKGLEWVARIRSKYNNY |
| | -VH | | ATYYADSVKERFTISRDDSKNTLYLQMNNLKTEDTAVYYCVRHRNFGNSYLSWFAYWG |
| | | | QGTLVTVSS |
| 557 | E2M_HC_A89L- | artificial | QTVVTQEPSLTVSPGGTVTLTCRSSTGAVTSGYYPNWVQQKPGQAPRGLIGATDMRPS |
| | VL | | GTPARFSGSLLGGKAALTLSGVQPEDEAEYYCALWYSNRWVFGGGTKLTVL |
| 558 | E2M_HC_A89T | artificial | EVQLVESGGGLVQPGGSLKLSCAASGFTFNGYAMNWVRQAPGKGLEWVARIRSKYNNY |
| | -VH | | ATYYADSVKERFTISRDDSKNTTYLQMNNLKTEDTAVYYCVRHRNFGNSYLSWFAYWG |
| | | | QGTLVTVSS |
| 559 | E2M_HC_A89T | artificial | QTVVTQEPSLTVSPGGTVTLTCRSSTGAVTSGYYPNWVQQKPGQAPRGLIGATDMRPS |
| | -VL | | GTPARFSGSLLGGKAALTLSGVQPEDEAEYYCALWYSNRWVFGGGTKLTVL |
| 560 | E2M_HC_A89V | artificial | EVQLVESGGGLVQPGGSLKLSCAASGFTFNGYAMNWVRQAPGKGLEWVARIRSKYNNY |
| | -VH | | ATYYADSVKERFTISRDDSKNTVYLQMNNLKTEDTAVYYCVRHRNFGNSYLSWFAYWG |
| | | | QGTLVTVSS |
| 561 | E2M_HC_A89V | artificial | QTVVTQEPSLTVSPGGTVTLTCRSSTGAVTSGYYPNWVQQKPGQAPRGLIGATDMRPS |
| | -VL | | GTPARFSGSLLGGKAALTLSGVQPEDEAEYYCALWYSNRWVFGGGTKLTVL |
| 562 | E2M_HC_L52M | artificial | EVQLVESGGGLVQPGGSLKLSCAASGFTFNGYAMNWVRQAPGKGMEWVARIRSKYNNY |
| | -VH | | ATYYADSVKERFTISRDDSKNTAYLQMNNLKTEDTAVYYCVRHRNFGNSYLSWFAYWG |
| | | | QGTLVTVSS |
| 563 | E2M_HC_L52M | artificial | QTVVTQEPSLTVSPGGTVTLTCRSSTGAVTSGYYPNWVQQKPGQAPRGLIGATDMRPS |
| | -VL | | GTPARFSGSLLGGKAALTLSGVQPEDEAEYYCALWYSNRWVFGGGTKLTVL |
| 564 | E2M_HC_L52M_ | artificial | EVQLVESGGGLVQPGGSLKLSCAASGFTFNGYAMNWVRQAPGKGMEWVARIRSKYNNY |
| | LC_L21I_ | | ATYYADSVKERFTISRDDSKNTAYLQMNNLKTEDTAVYYCVRHRNFGNSYLSWFAYWG |
| | Q46K_G141S- | | QGTLVTVSS |
| | VH | | |
| 565 | E2M_HC_L52M_ | artificial | QTVVTQEPSLTVSPGGTVTITCRSSTGAVTSGYYPNWVQKKPGQAPRGLIGATDMRPS |
| | LC_L21I_ | | GTPARFSGSLLGGKAALTLSGVQPEDEAEYYCALWYSNRWVFGSGTKLTVL |
| | 6Q4K_G141S- | | |
| | VL | | |
| 566 | E2M_HC_L52M_ | artificial | EVQLVESGGGLVQPGGSLKLSCAASGFTFNGYAMNWVRQAPGKGMEWVARIRSKYNNY |
| | LC_Q46K- | | ATYYADSVKERFTISRDDSKNTAYLQMNNLKTEDTAVYYCVRHRNFGNSYLSWFAYWG |
| | VH | | QGTLVTVSS |
| 567 | E2M_HC_L52M_ | artificial | QTVVTQEPSLTVSPGGTVTLTCRSSTGAVTSGYYPNWVQKKPGQAPRGLIGATDMRPS |
| | LC_Q46K- | | GTPARFSGSLLGGKAALTLSGVQPEDEAEYYCALWYSNRWVFGGGTKLTVL |
| | VL | | |
| 568 | E2M_HC_L52M_ | artificial | EVQLVESGGGLVQPGGSLKLSCAASGFTFNGYAMNWVRQAPGKGMEWVARIRSKYNNY |
| | LC_Q46K_ | | ATYYADSVKERFTISRDDSKNTAYLQMNNLKTEDTAVYYCVRHRNFGNSYLSWFAYWG |
| | G141S-VH | | QGTLVTVSS |
| 569 | E2M_HC_L52M_ | artificial | QTVVTQEPSLTVSPGGTVTLTCRSSTGAVTSGYYPNWVQKKPGQAPRGLIGATDMRPS |
| | LC_Q46K_ | | GTPARFSGSLLGGKAALTLSGVQPEDEAEYYCALWYSNRWVFGSGTKLTVL |
| | G141S-VL | | |
| 570 | E2M_HC_L52V | artificial | EVQLVESGGGLVQPGGSLKLSCAASGFTFNGYAMNWVRQAPGKGVEWVARIRSKYNNY |
| | -VH | | ATYYADSVKERFTISRDDSKNTAYLQMNNLKTEDTAVYYCVRHRNFGNSYLSWFAYWG |
| | | | QGTLVTVSS |
| 571 | E2M_HC_L52V | artificial | QTVVTQEPSLTVSPGGTVTLTCRSSTGAVTSGYYPNWVQQKPGQAPRGLIGATDMRPS |
| | -VL | | GTPARFSGSLLGGKAALTLSGVQPEDEAEYYCALWYSNRWVFGGGTKLTVL |
| 572 | E2M_HC_L52V_ | artificial | EVQLVESGGGLVQPGGSLKLSCAASGFTFNGYAMNWVRQAPGKGVEWVARIRSKYNNY |
| | LC_V44F- | | ATYYADSVKERFTISRDDSKNTAYLQMNNLKTEDTAVYYCVRHRNFGNSYLSWFAYWG |
| | VH | | QGTLVTVSS |
| 573 | E2M_HC_L52V_ | artificial | QTVVTQEPSLTVSPGGTVTLTCRSSTGAVTSGYYPNWFQQKPGQAPRGLIGATDMRPS |
| | LC_V44F- | | GTPARFSGSLLGGKAALTLSGVQPEDEAEYYCALWYSNRWVFGGGTKLTVL |
| | VL | | |
| 574 | E2M_HC_L52V_ | artificial | EVQLVESGGGLVQPGGSLKLSCAASGFTFNGYAMNWVRQAPGKGVEWVARIRSKYNNY |
| | LC_V44Y- | | ATYYADSVKERFTISRDDSKNTAYLQMNNLKTEDTAVYYCVRHRNFGNSYLSWFAYWG |
| | VH | | QGTLVTVSS |
| 575 | E2M_HC_L52V_ | artificial | QTVVTQEPSLTVSPGGTVTLTCRSSTGAVTSGYYPNWYQQKPGQAPRGLIGATDMRPS |
| | LC_V44Y- | | GTPARFSGSLLGGKAALTLSGVQPEDEAEYYCALWYSNRWVFGGGTKLTVL |
| | VL | | |
| 576 | E2M_HC_M41F_ | artificial | GYAFN |
| | A89V- | | |
| | HCDR1 | | |
| 577 | E2M_HC_M41F_ | artificial | RIRSKYNNYATYYADSVKE |
| | A89V- | | |
| | HCDR2 | | |
| 578 | E2M_HC_M41F_ | artificial | HRNFGNSYLSWFAY |
| | A89V-HCDR3 | | |
| 579 | E2M_HC_M41F_ | artificial | RSSTGAVTSGYYPN |
| | A89V-LCDR1 | | |
| 580 | E2M_HC_M41F_ | artificial | ATDMRPS |
| | A89V-LCDR2 | | |
| 581 | E2M_HC_M41F_ | artificial | ALWYSNRWV |
| | A89V- | | |
| | LCDR3 | | |
| 582 | E2M_HC_M41F_ | artificial | EVQLVESGGGLVQPGGSLKLSCAASGFTFNGYAFNWVRQAPGKGLEWVARIRSKYNNY |
| | A89V-VH | | ATYYADSVKERFTISRDDSKNTVYLQMNNLKTEDTAVYYCVRHRNFGNSYLSWFAYWG |
| | | | QGTLVTVSS |
| 583 | E2M_HC_M41F_ | artificial | QTVVTQEPSLTVSPGGTVTLTCRSSTGAVTSGYYPNWVQQKPGQAPRGLIGATDMRPS |
| | A89V-VL | | GTPARFSGSLLGGKAALTLSGVQPEDEAEYYCALWYSNRWVFGGGTKLTVL |
| 584 | E2M_HC_M41I | artificial | GYAIN |
| | -HCDR1 | | |
| 585 | E2M_HC_M41I | artificial | RIRSKYNNYATYYADSVKE |
| | -HCDR2 | | |
| 586 | E2M_HC_M41I | artificial | HRNFGNSYLSWFAY |
| | -HCDR3 | | |
| 587 | E2M_HC_M41I | artificial | RSSTGAVTSGYYPN |
| | -LCDR1 | | |
| 588 | E2M_HC_M41I | artificial | ATDMRPS |
| | -LCDR2 | | |
| 589 | E2M_HC_M41I | artificial | ALWYSNRWV |
| | -LCDR3 | | |
| 590 | E2M_HC_M41I | artificial | EVQLVESGGGLVQPGGSLKLSCAASGFTFNGYAINWVRQAPGKGLEWVARIRSKYNNY |
| | -VH | | ATYYADSVKERFTISRDDSKNTAYLQMNNLKTEDTAVYYCVRHRNFGNSYLSWFAYWG |
| | | | QGTLVTVSS |
| 591 | E2M_HC_M41I | artificial | QTVVTQEPSLTVSPGGTVTLTCRSSTGAVTSGYYPNWVQQKPGQAPRGLIGATDMRPS |
| | -VL | | GTPARFSGSLLGGKAALTLSGVQPEDEAEYYCALWYSNRWVFGGGTKLTVL |
| 592 | E2M_HC_M41I_ | artificial | EVQLVESGGGLVQPGGSLKLSCAASGFTFNGYAINWVRQAPGKGLEWVARIRSKYNNY |
| | A89L-VH | | ATYYADSVKERFTISRDDSKNTLYLQMNNLKTEDTAVYYCVRHRNFGNSYLSWFAYWG |
| | | | QGTLVTVSS |
| 593 | E2M_HC_M41I_ | artificial | QTVVTQEPSLTVSPGGTVTLTCRSSTGAVTSGYYPNWVQQKPGQAPRGLIGATDMRPS |
| | A89L-VL | | GTPARFSGSLLGGKAALTLSGVQPEDEAEYYCALWYSNRWVFGGGTKLTVL |
| 594 | E2M_HC_M41I_ | artificial | EVQLVESGGGLVQPGGSLKLSCAASGFTENGYAINWVRQAPGKGLEWVARIRSKYNNY |
| | A89V-VH | | ATYYADSVKERFTISRDDSKNTVYLQMNNLKTEDTAVYYCVRHRNFGNSYLSWFAYWG |
| | | | QGTLVTVSS |
| 595 | E2M_HC_M41I_ | artificial | QTVVTQEPSLTVSPGGTVTLTCRSSTGAVTSGYYPNWVQQKPGQAPRGLIGATDMRPS |
| | A89V-VL | | GTPARFSGSLLGGKAALTLSGVQPEDEAEYYCALWYSNRWVFGGGTKLTVL |
| 596 | E2M_HC_M41I_ | artificial | EVQLVESGGGLVQPGGSLKLSCAASGFTFNGYAINWVRQAPGKGLEWVARIRSKYNNY |
| | A89V_LC_ | | ATYYADSVKERFTISRDDSKNTVYLQMNNLKTEDTAVYYCVRHRNFGNSYLSWFAYWG |
| | L21I_Q46K_ | | QGTLVTVSS |
| | 1G14S-VH | | |
| 597 | E2M_HC_M41I_ | artificial | QTVVTQEPSLTVSPGGTVTITCRSSTGAVTSGYYPNWVQKKPGQAPRGLIGATDMRPS |
| | A89V_LC_ | | GTPARFSGSLLGGKAALTLSGVQPEDEAEYYCALWYSNRWVFGSGTKLTVL |
| | L21I_Q46K_ | | |
| | G141S-VL | | |
| 598 | E2M_HC_M41I_ | artificial | EVQLVESGGGLVQPGGSLKLSCAASGFTFNGYAINWVRQAPGKGLEWVARIRSKYNNY |
| | A89V_LC_ | | ATYYADSVKERFTISRDDSKNTVYLQMNNLKTEDTAVYYCVRHRNFGNSYLSWFAYWG |
| | 6Q4K-VH | | QGTLVTVSS |
| 599 | E2M_HC_M41I_ | artificial | QTVVTQEPSLTVSPGGTVTLTCRSSTGAVTSGYYPNWVQKKPGQAPRGLIGATDMRPS |
| | A89V_LC_ | | GTPARFSGSLLGGKAALTLSGVQPEDEAEYYCALWYSNRWVFGGGTKLTVL |
| | Q46K-VL | | |
| 600 | E2M_HC_M41I_ | artificial | EVQLVESGGGLVQPGGSLKLSCAASGFTFNGYAINWVRQAPGKGVEWVARIRSKYNNY |
| | L52V_A89V_ | | ATYYADSVKERFTISRDDSKNTVYLQMNNLKTEDTAVYYCVRHRNFGNSYLSWFAYWG |
| | LC_L21I_ | | QGTLVTVSS |
| | Q46K_G141S_ | | |
| | V44F-VH | | |
| 601 | E2M_HC_M41I_ | artificial | QTVVTQEPSLTVSPGGTVTITCRSSTGAVTSGYYPNWFQKKPGQAPRGLIGATDMRPS |
| | L52V_A89V_ | | GTPARFSGSLLGGKAALTLSGVQPEDEAEYYCALWYSNRWVFGSGTKLTVL |
| | LC_L21I_ | | |
| | Q46K_G141S_ | | |
| | FV44-VL | | |
| 602 | E2M_HC_M41I_ | artificial | EVQLVESGGGLVQPGGSLKLSCAASGFTFNGYAINWVRQAPGKGLEWVARIRSKYNNY |
| | LC_L21I_ | | ATYYADSVKERFTISRDDSKNTAYLQMNNLKTEDTAVYYCVRHRNFGNSYLSWFAYWG |
| | Q46K_G141S- | | QGTLVTVSS |
| | VH | | |
| 603 | E2M_HC_M41I_ | artificial | QTVVTQEPSLTVSPGGTVTITCRSSTGAVTSGYYPNWVQKKPGQAPRGLIGATDMRPS |
| | LC_L21I_ | | GTPARFSGSLLGGKAALTLSGVQPEDEAEYYCALWYSNRWVFGSGTKLTVL |
| | Q46K_G141S- | | |
| | VL | | |
| 604 | E2M_HC_M41I_ | artificial | EVQLVESGGGLVQPGGSLKLSCAASGFTFNGYAINWVREAPGKGLEWVARIRSKYNNY |
| | Q46E_A89V_ | | ATYYADSVKERFTISRDDSKNTVYLQMNNLKTEDTAVYYCVRHRNFGNSYLSWFAYWG |
| | LC_L21I_ | | QGTLVTVSS |
| | Q46K_G141S- | | |
| | VH | | |
| 605 | E2M_HC_M41I_ | artificial | QTVVTQEPSLTVSPGGTVTITCRSSTGAVTSGYYPNWVQKKPGQAPRGLIGATDMRPS |
| | Q46E_A89V_ | | GTPARFSGSLLGGKAALTLSGVQPEDEAEYYCALWYSNRWVFGSGTKLTVL |
| | LC_L21I_ | | |
| | Q46K_G141S- | | |
| | VL | | |
| 606 | E2M_HC_M41V | artificial | GYAVN |
| | -HCDR1 | | |
| 607 | E2M_HC_M41V | artificial | RIRSKYNNYATYYADSVKE |
| | -HCDR2 | | |
| 608 | E2M_HC_M41V | artificial | HRNFGNSYLSWFAY |
| | -HCDR3 | | |
| 609 | E2M_HC_M41V | artificial | RSSTGAVTSGYYPN |
| | -LCDR1 | | |
| 610 | E2M_HC_M41V | artificial | ATDMRPS |
| | -LCDR2 | | |
| 611 | E2M_HC_M41V | artificial | ALWYSNRWV |
| | -LCDR3 | | |
| 612 | E2M_HC_M41V | artificial | EVQLVESGGGLVQPGGSLKLSCAASGFTFNGYAVNWVRQAPGKGLEWVARIRSKYNNY |
| | -VH | | ATYYADSVKERFTISRDDSKNTAYLQMNNLKTEDTAVYYCVRHRNFGNSYLSWFAYWG |
| | | | QGTLVTVSS |
| 613 | E2M_HC_M41V | artificial | QTVVTQEPSLTVSPGGTVTLTCRSSTGAVTSGYYPNWVQQKPGQAPRGLIGATDMRPS |
| | -VL | | GTPARFSGSLLGGKAALTLSGVQPEDEAEYYCALWYSNRWVFGGGTKLTVL |
| 614 | E2M_HC_Q46D | artificial | EVQLVESGGGLVQPGGSLKLSCAASGFTFNGYAMNWVRDAPGKGLEWVARIRSKYNNY |
| | -VH | | ATYYADSVKERFTISRDDSKNTAYLQMNNLKTEDTAVYYCVRHRNFGNSYLSWFAYWG |
| | | | QGTLVTVSS |
| 615 | E2M_HC_Q46D | artificial | QTVVTQEPSLTVSPGGTVTLTCRSSTGAVTSGYYPNWVQQKPGQAPRGLIGATDMRPS |
| | -VL | | GTPARFSGSLLGGKAALTLSGVQPEDEAEYYCALWYSNRWVFGGGTKLTVL |
| 616 | E2M_HC_Q46D_ | artificial | EVQLVESGGGLVQPGGSLKLSCAASGFTFNGYAMNWVRDAPGKGLEWVARIRSKYNNY |
| | LC_L21I_ | | ATYYADSVKERFTISRDDSKNTAYLQMNNLKTEDTAVYYCVRHRNFGNSYLSWFAYWG |
| | Q46K_G141S- | | QGTLVTVSS |
| | VH | | |
| 617 | E2M_HC_Q46D_ | artificial | QTVVTQEPSLTVSPGGTVTITCRSSTGAVTSGYYPNWVQKKPGQAPRGLIGATDMRPS |
| | LC_L21I_ | | GTPARFSGSLLGGKAALTLSGVQPEDEAEYYCALWYSNRWVFGSGTKLTVL |
| | 6Q4K_G141S- | | |
| | VL | | |
| 618 | E2M_HC_Q46D_ | artificial | EVQLVESGGGLVQPGGSLKLSCAASGFTFNGYAMNWVRDAPGKGLEWVARIRSKYNNY |
| | LC_L21I_ | | ATYYADSVKERFTISRDDSKNTAYLQMNNLKTEDTAVYYCVRHRNFGNSYLSWFAYWG |
| | Q46R_G141S- | | QGTLVTVSS |
| | VH | | |
| 619 | E2M_HC_Q46D_ | artificial | QTVVTQEPSLTVSPGGTVTITCRSSTGAVTSGYYPNWVQRKPGQAPRGLIGATDMRPS |
| | LC_L21I_ | | GTPARFSGSLLGGKAALTLSGVQPEDEAEYYCALWYSNRWVFGSGTKLTVL |
| | Q46R_G141S- | | |
| | VL | | |
| 620 | E2M_HC_Q46D_ | artificial | EVQLVESGGGLVQPGGSLKLSCAASGFTFNGYAMNWVRDAPGKGLEWVARIRSKYNNY |
| | LC_Q46K- | | ATYYADSVKERFTISRDDSKNTAYLQMNNLKTEDTAVYYCVRHRNFGNSYLSWFAYWG |
| | VH | | QGTLVTVSS |
| 621 | E2M_HC_Q46D_ | artificial | QTVVTQEPSLTVSPGGTVTLTCRSSTGAVTSGYYPNWVQKKPGQAPRGLIGATDMRPS |
| | LC_Q46K- | | GTPARFSGSLLGGKAALTLSGVQPEDEAEYYCALWYSNRWVFGGGTKLTVL |
| | VL | | |
| 622 | E2M_HC_Q46D_ | artificial | EVQLVESGGGLVQPGGSLKLSCAASGFTFNGYAMNWVRDAPGKGLEWVARIRSKYNNY |
| | LC_Q46K_ | | ATYYADSVKERFTISRDDSKNTAYLQMNNLKTEDTAVYYCVRHRNFGNSYLSWFAYWG |
| | G141S-VH | | QGTLVTVSS |
| 623 | E2M_HC_Q46D_ | artificial | QTVVTQEPSLTVSPGGTVTLTCRSSTGAVTSGYYPNWVQKKPGQAPRGLIGATDMRPS |
| | LC_Q46K_ | | GTPARFSGSLLGGKAALTLSGVQPEDEAEYYCALWYSNRWVFGSGTKLTVL |
| | G141S-VL | | |
| 624 | E2M_HC_Q46D_ | artificial | EVQLVESGGGLVQPGGSLKLSCAASGFTFNGYAMNWVRDAPGKGLEWVARIRSKYNNY |
| | LC_Q46R- | | ATYYADSVKERFTISRDDSKNTAYLQMNNLKTEDTAVYYCVRHRNFGNSYLSWFAYWG |
| | VH | | QGTLVTVSS |
| 625 | E2M_HC_Q46D_ | artificial | QTVVTQEPSLTVSPGGTVTLTCRSSTGAVTSGYYPNWVQRKPGQAPRGLIGATDMRPS |
| | LC_Q46R- | | GTPARFSGSLLGGKAALTLSGVQPEDEAEYYCALWYSNRWVFGGGTKLTVL |
| | VL | | |
| 626 | E2M_HC_Q46D_ | artificial | EVQLVESGGGLVQPGGSLKLSCAASGFTFNGYAMNWVRDAPGKGLEWVARIRSKYNNY |
| | LC_Q46R_ | | ATYYADSVKERFTISRDDSKNTAYLQMNNLKTEDTAVYYCVRHRNFGNSYLSWFAYWG |
| | 4G11S-VH | | QGTLVTVSS |
| 627 | E2M_HC_Q46D_ | artificial | QTVVTQEPSLTVSPGGTVTLTCRSSTGAVTSGYYPNWVQRKPGQAPRGLIGATDMRPS |
| | LC_Q46R_ | | GTPARFSGSLLGGKAALTLSGVQPEDEAEYYCALWYSNRWVFGSGTKLTVL |
| | G141S-VL | | |
| 628 | E2M_HC_Q46E | artificial | EVQLVESGGGLVQPGGSLKLSCAASGFTFNGYAMNWVREAPGKGLEWVARIRSKYNNY |
| | -VH | | ATYYADSVKERFTISRDDSKNTAYLQMNNLKTEDTAVYYCVRHRNFGNSYLSWFAYWG |
| | | | QGTLVTVSS |
| 629 | E2M_HC_Q46E | artificial | QTVVTQEPSLTVSPGGTVTLTCRSSTGAVTSGYYPNWVQQKPGQAPRGLIGATDMRPS |
| | -VL | | GTPARFSGSLLGGKAALTLSGVQPEDEAEYYCALWYSNRWVFGGGTKLTVL |
| 630 | E2M_HC_Q46E_ | artificial | EVQLVESGGGLVQPGGSLKLSCAASGFTFNGYAMNWVREAPGKGLEWVARIRSKYNNY |
| | LC_L21I_ | | ATYYADSVKERFTISRDDSKNTAYLQMNNLKTEDTAVYYCVRHRNFGNSYLSWFAYWG |
| | Q46K_G141S- | | QGTLVTVSS |
| | VH | | |
| 631 | E2M_HC_Q46E_ | artificial | QTVVTQEPSLTVSPGGTVTITCRSSTGAVTSGYYPNWVQKKPGQAPRGLIGATDMRPS |
| | LC_L21I_ | | GTPARFSGSLLGGKAALTLSGVQPEDEAEYYCALWYSNRWVFGSGTKLTVL |
| | Q46K_G141S- | | |
| | VL | | |
| 632 | E2M_HC_Q46E_ | artificial | EVQLVESGGGLVQPGGSLKLSCAASGFTFNGYAMNWVREAPGKGLEWVARIRSKYNNY |
| | LC_L21I_ | | ATYYADSVKERFTISRDDSKNTAYLQMNNLKTEDTAVYYCVRHRNFGNSYLSWFAYWG |
| | Q46R_G141S- | | QGTLVTVSS |
| | VH | | |
| 633 | E2M_HC_Q46E_ | artificial | QTVVTQEPSLTVSPGGTVTITCRSSTGAVTSGYYPNWVQRKPGQAPRGLIGATDMRPS |
| | LC_L21I_ | | GTPARFSGSLLGGKAALTLSGVQPEDEAEYYCALWYSNRWVFGSGTKLTVL |
| | Q46R_G141S- | | |
| | VL | | |
| 634 | E2M_HC_Q46E_ | artificial | EVQLVESGGGLVQPGGSLKLSCAASGFTFNGYAMNWVREAPGKGLEWVARIRSKYNNY |
| | LC_Q46K- | | ATYYADSVKERFTISRDDSKNTAYLQMNNLKTEDTAVYYCVRHRNFGNSYLSWFAYWG |
| | VH | | QGTLVTVSS |
| 635 | E2M_HC_Q46E_ | artificial | QTVVTQEPSLTVSPGGTVTLTCRSSTGAVTSGYYPNWVQKKPGQAPRGLIGATDMRPS |
| | LC_Q46K- | | GTPARFSGSLLGGKAALTLSGVQPEDEAEYYCALWYSNRWVFGGGTKLTVL |
| | VL | | |
| 636 | E2M_HC_Q46E_ | artificial | EVQLVESGGGLVQPGGSLKLSCAASGFTFNGYAMNWVREAPGKGLEWVARIRSKYNNY |
| | LC_Q46K_ | | ATYYADSVKERFTISRDDSKNTAYLQMNNLKTEDTAVYYCVRHRNFGNSYLSWFAYWG |
| | G141S-VH | | QGTLVTVSS |
| 637 | E2M_HC_Q46E_ | artificial | QTVVTQEPSLTVSPGGTVTLTCRSSTGAVTSGYYPNWVQKKPGQAPRGLIGATDMRPS |
| | LC_Q46K_ | | GTPARFSGSLLGGKAALTLSGVQPEDEAEYYCALWYSNRWVFGSGTKLTVL |
| | G141S-VL | | |
| 638 | E2M_HC_Q46E_ | artificial | EVQLVESGGGLVQPGGSLKLSCAASGFTFNGYAMNWVREAPGKGLEWVARIRSKYNNY |
| | LC_Q46R- | | ATYYADSVKERFTISRDDSKNTAYLQMNNLKTEDTAVYYCVRHRNFGNSYLSWFAYWG |
| | VH | | QGTLVTVSS |
| 639 | E2M_HC_Q46E_ | artificial | QTVVTQEPSLTVSPGGTVTLTCRSSTGAVTSGYYPNWVQRKPGQAPRGLIGATDMRPS |
| | LC_Q46R- | | GTPARFSGSLLGGKAALTLSGVQPEDEAEYYCALWYSNRWVFGGGTKLTVL |
| | VL | | |
| 640 | E2M_HC_Q46E_ | artificial | EVQLVESGGGLVQPGGSLKLSCAASGFTFNGYAMNWVREAPGKGLEWVARIRSKYNNY |
| | LC_Q46R_ | | ATYYADSVKERFTISRDDSKNTAYLQMNNLKTEDTAVYYCVRHRNFGNSYLSWFAYWG |
| | G141S-VH | | QGTLVTVSS |
| 641 | E2M_HC_Q46E_ | artificial | QTVVTQEPSLTVSPGGTVTLTCRSSTGAVTSGYYPNWVQRKPGQAPRGLIGATDMRPS |
| | LC_Q46R_ | | GTPARFSGSLLGGKAALTLSGVQPEDEAEYYCALWYSNRWVFGSGTKLTVL |
| | G141S-VL | | |
| 642 | E2M_HC_Q46K | artificial | EVQLVESGGGLVQPGGSLKLSCAASGFTFNGYAMNWVRKAPGKGLEWVARIRSKYNNY |
| | -VH | | ATYYADSVKERFTISRDDSKNTAYLQMNNLKTEDTAVYYCVRHRNFGNSYLSWFAYWG |
| | | | QGTLVTVSS |
| 643 | E2M_HC_Q46K | artificial | QTVVTQEPSLTVSPGGTVTLTCRSSTGAVTSGYYPNWVQQKPGQAPRGLIGATDMRPS |
| | -VL | | GTPARFSGSLLGGKAALTLSGVQPEDEAEYYCALWYSNRWVFGGGTKLTVL |
| 644 | E2M_HC_Q46R | artificial | EVQLVESGGGLVQPGGSLKLSCAASGFTFNGYAMNWVRRAPGKGLEWVARIRSKYNNY |
| | -VH | | ATYYADSVKERFTISRDDSKNTAYLQMNNLKTEDTAVYYCVRHRNFGNSYLSWFAYWG |
| | | | QGTLVTVSS |
| 645 | E2M_HC_Q46R | artificial | QTVVTQEPSLTVSPGGTVTLTCRSSTGAVTSGYYPNWVQQKPGQAPRGLIGATDMRPS |
| | -VL | | GTPARFSGSLLGGKAALTLSGVQPEDEAEYYCALWYSNRWVFGGGTKLTVL |
| 646 | E2M_HC_V107A- | artificial | EVQLVESGGGLVQPGGSLKLSCAASGFTFNGYAMNWVRQAPGKGLEWVARIRSKYNNY |
| | VH | | ATYYADSVKERFTISRDDSKNTAYLQMNNLKTEDTAVYYCARHRNFGNSYLSWFAYWG |
| | | | QGTLVTVSS |
| 647 | E2M_HC_V107A- | artificial | QTVVTQEPSLTVSPGGTVTLTCRSSTGAVTSGYYPNWVQQKPGQAPRGLIGATDMRPS |
| | VL | | GTPARFSGSLLGGKAALTLSGVQPEDEAEYYCALWYSNRWVFGGGTKLTVL |
| 648 | E2M_HC_V107L- | artificial | EVQLVESGGGLVQPGGSLKLSCAASGFTFNGYAMNWVRQAPGKGLEWVARIRSKYNNY |
| | VH | | ATYYADSVKERFTISRDDSKNTAYLQMNNLKTEDTAVYYCLRHRNFGNSYLSWFAYWG |
| | | | QGTLVTVSS |
| 649 | E2M_HC_V107L- | artificial | QTVVTQEPSLTVSPGGTVTLTCRSSTGAVTSGYYPNWVQQKPGQAPRGLIGATDMRPS |
| | VL | | GTPARFSGSLLGGKAALTLSGVQPEDEAEYYCALWYSNRWVFGGGTKLTVL |
| 650 | E2M_I2E- | artificial | GYAIN |
| | HCDR1 | | |
| 651 | E2M_I2E- | artificial | RIRSKYNNYATYYADAVKE |
| | HCDR2 | | |
| 652 | E2M_I2E- | artificial | ARNFGSSYLSWFAY |
| | HCDR3 | | |
| 653 | E2M_I2E- | artificial | RSSTGAVTSGYYPN |
| | LCDR1 | | |
| 654 | E2M_I2E- | artificial | ATDMRPS |
| | LCDR2 | | |
| 655 | E2M_I2E- | artificial | ALWYSNRWV |
| | LCDR3 | | |
| 656 | E2M_I2E- | artificial | EVQLVESGGGLVQPGGSLKLSCAASGFTFNGYAINWVRQAPGKGLEWVARIRSKYNNY |
| | VH | | ATYYADAVKERFTISRDDSKNTVYLQMNNLKTEDTAVYYCARARNFGSSYLSWFAYWG |
| | | | QGTLVTVSS |
| 657 | E2M_I2E- | artificial | QTVVTQEPSLTVSPGGTVTITCRSSTGAVTSGYYPNWVQKKPGQAPRGLIGATDMRPS |
| | VL | | GTPARFSGSLSGGKAALTLSGVQPEDEAEYYCALWYSNRWVFGSGTKLTVL |
| 658 | E2M_I2K- | artificial | GYAMN |
| | HCDR1 | | |
| 659 | E2M_I2K- | artificial | RIRSKYNNYATYYAEAVKG |
| | HCDR2 | | |
| 660 | E2M_I2K- | artificial | NENIGTSYLSWFAY |
| | HCDR3 | | |
| 661 | E2M_I2K- | artificial | RSSTGAVTSGYYPN |
| | LCDR1 | | |
| 662 | E2M_I2K- | artificial | ATDMRPS |
| | LCDR2 | | |
| 663 | E2M_I2K- | artificial | ALWYSNRWV |
| | LCDR3 | | |
| 664 | E2M_I2K- | artificial | EVQLVESGGGLVQPGGSLKLSCAASGFTFNGYAMNWVRQAPGKGLEWVARIRSKYNNY |
| | VH | | ATYYAEAVKGRFTISRDDSKNTVYLQMNNLKTEDTAVYYCVRNENIGTSYLSWFAYWG |
| | | | QGTLVTVSS |
| 665 | E2M_I2K- | artificial | QTVVTQEPSLTVSPGGTVTITCRSSTGAVTSGYYPNWVQQKPGQAPRGLIGATDMRPS |
| | VL | | GTPARFSGSLSGGKAALTLSGVQPEDEAEYYCALWYSNRWVFGSGTKLTVL |
| 666 | E2M_I2L- | artificial | GYAMN |
| | HCDR1 | | |
| 667 | E2M_I2L- | artificial | RIRSKYNNYATYYAEAVKE |
| | HCDR2 | | |
| 668 | E2M_I2L- | artificial | ARNFGSSYLSWFAY |
| | HCDR3 | | |
| 669 | E2M_I2L- | artificial | RSSTGAVTSGYYPN |
| | LCDR1 | | |
| 670 | E2M_I2L- | artificial | ATDMRPS |
| | LCDR2 | | |
| 671 | E2M_I2L- | artificial | ALYYSNRWV |
| | LCDR3 | | |
| 672 | E2M_I2L- | artificial | EVQLVESGGGLVQPGGSLKLSCAASGFTFNGYAMNWVRQAPGKGMEWVARIRSKYNNY |
| | VH | | ATYYADAVKERFTISRDDSKNTLYLQMNNLKTEDTAVYYCVRARNFGSSYLSWFAYWG |
| | | | QGTLVTVSS |
| 673 | E2M_I2L- | artificial | QTVVTQEPSLTVSPGGTVTITCRSSTGAVTSGYYPNWIQKKPGQAPRGLIGATDMRPS |
| | VL | | GTPARFSGSLEGGKAALTLSGVQPEDEAEYYCALYYSNRWVFGSGTKLTVL |
| 674 | E2M_I2M- | artificial | GYAMN |
| | HCDR1 | | |
| 675 | E2M_I2M- | artificial | RIRSKYNNYATYYADAVKE |
| | HCDR2 | | |
| 676 | E2M_I2M- | artificial | ARNFGTSYLSWFAY |
| | HCDR3 | | |
| 677 | E2M_I2M- | artificial | RSSTGAVTSGYYPN |
| | LCDR1 | | |
| 678 | E2M_I2M- | artificial | ATDMRPS |
| | LCDR2 | | |
| 679 | E2M_I2M- | artificial | ALWYSNRWV |
| | LCDR3 | | |
| 680 | E2M_I2M- | artificial | EVQLVESGGGLVQPGGSLKLSCAASGFTFNGYAMNWVRQAPGKGMEWVARIRSKYNNY |
| | VH | | ATYYADAVKERFTISRDDSKNTLYLQMNNLKTEDTAVYYCVRARNFGTSYLSWFAYWG |
| | | | QGTLVTVSS |
| 681 | E2M_I2M- | artificial | QTVVTQEPSLTVSPGGTVTITCRSSTGAVTSGYYPNWVQKKPGQAPRGLIGATDMRPS |
| | VL | | GTPARFSGSLSGGKAALTLSGVQPEDEAEYYCALWYSNRWVFGSGTKLTVL |
| 682 | E2M_I2M2- | artificial | GYAIN |
| | HCDR1 | | |
| 683 | E2M_I2M2- | artificial | RIRSKYNNYATYYADAVKE |
| | HCDR2 | | |
| 684 | E2M_I2M2- | artificial | NANFGTSYLSFFAY |
| | HCDR3 | | |
| 685 | E2M_I2M2- | artificial | RSSTGAVTSGYYPN |
| | LCDR1 | | |
| 686 | E2M_I2M2- | artificial | ATDMRPS |
| | LCDR2 | | |
| 687 | E2M_I2M2- | artificial | ALWYSNRWV |
| | LCDR3 | | |
| 688 | E2M_I2M2- | artificial | EVQLVESGGGLVQPGGSLKLSCAASGFTENGYAINWVREAPGKGLEWVARIRSKYNNY |
| | VH | | ATYYADAVKERFTISRDDSKNTAYLQMNNLKTEDTAVYYCVRNANFGTSYLSYFAYWG |
| | | | QGTLVTVSS |
| 689 | E2M_I2M2- | artificial | QTVVTQEPSLTVSPGGTVTLTCRSSTGAVTSGYYPNWVQKKPGQAPRGLIGATDMRPS |
| | VL | | GTPARFSGSLLGGKAALTLSGVQPEDEAEYYCALWYSNRWVFGSGTKLTVL |
| 690 | E2M_LC_G141S- | artificial | EVQLVESGGGLVQPGGSLKLSCAASGFTFNGYAMNWVRQAPGKGLEWVARIRSKYNNY |
| | VH | | ATYYADSVKERFTISRDDSKNTAYLQMNNLKTEDTAVYYCVRHRNFGNSYLSWFAYWG |
| | | | QGTLVTVSS |
| 691 | E2M_LC_G141S- | artificial | QTVVTQEPSLTVSPGGTVTLTCRSSTGAVTSGYYPNWVQQKPGQAPRGLIGATDMRPS |
| | VL | | GTPARFSGSLLGGKAALTLSGVQPEDEAEYYCALWYSNRWVFGSGTKLTVL |
| 692 | E2M_LC_G141S_ | artificial | EVQLVESGGGLVQPGGSLKLSCAASGFTFNGYAMNWVRQAPGKGLEWVARIRSKYNNY |
| | L21I-VH | | ATYYADSVKERFTISRDDSKNTAYLQMNNLKTEDTAVYYCVRHRNFGNSYLSWFAYWG |
| | | | QGTLVTVSS |
| 693 | E2M_LC_G141S_ | artificial | QTVVTQEPSLTVSPGGTVTITCRSSTGAVTSGYYPNWVQQKPGQAPRGLIGATDMRPS |
| | L21I-VL | | GTPARFSGSLLGGKAALTLSGVQPEDEAEYYCALWYSNRWVFGSGTKLTVL |
| 694 | E2M_LC_L83E | artificial | EVQLVESGGGLVQPGGSLKLSCAASGFTFNGYAMNWVRQAPGKGLEWVARIRSKYNNY |
| | -VH | | ATYYADSVKERFTISRDDSKNTAYLQMNNLKTEDTAVYYCVRHRNFGNSYLSWFAYWG |
| | | | QGTLVTVSS |
| 695 | E2M_LC_L83E | artificial | QTVVTQEPSLTVSPGGTVTLTCRSSTGAVTSGYYPNWVQQKPGQAPRGLIGATDMRPS |
| | -VL | | GTPARFSGSLEGGKAALTLSGVQPEDEAEYYCALWYSNRWVFGGGTKLTVL |
| 696 | E2M_LC_L83S | artificial | EVQLVESGGGLVQPGGSLKLSCAASGFTFNGYAMNWVRQAPGKGLEWVARIRSKYNNY |
| | -VH | | ATYYADSVKERFTISRDDSKNTAYLQMNNLKTEDTAVYYCVRHRNFGNSYLSWFAYWG |
| | | | QGTLVTVSS |
| 697 | E2M_LC_L83S | artificial | QTVVTQEPSLTVSPGGTVTLTCRSSTGAVTSGYYPNWVQQKPGQAPRGLIGATDMRPS |
| | -VL | | GTPARFSGSLSGGKAALTLSGVQPEDEAEYYCALWYSNRWVFGGGTKLTVL |
| 698 | E2M_LC_Q46E | artificial | EVQLVESGGGLVQPGGSLKLSCAASGFTFNGYAMNWVRQAPGKGLEWVARIRSKYNNY |
| | -VH | | ATYYADSVKERFTISRDDSKNTAYLQMNNLKTEDTAVYYCVRHRNFGNSYLSWFAYWG |
| | | | QGTLVTVSS |
| 699 | E2M_LC_Q46E | artificial | QTVVTQEPSLTVSPGGTVTLTCRSSTGAVTSGYYPNWVQEKPGQAPRGLIGATDMRPS |
| | -VL | | GTPARFSGSLLGGKAALTLSGVQPEDEAEYYCALWYSNRWVFGGGTKLTVL |
| 700 | E2M_LC_Q46K | artificial | EVQLVESGGGLVQPGGSLKLSCAASGFTFNGYAMNWVRQAPGKGLEWVARIRSKYNNY |
| | -VH | | ATYYADSVKERFTISRDDSKNTAYLQMNNLKTEDTAVYYCVRHRNFGNSYLSWFAYWG |
| | | | QGTLVTVSS |
| 701 | E2M_LC_Q46K | artificial | QTVVTQEPSLTVSPGGTVTLTCRSSTGAVTSGYYPNWVQKKPGQAPRGLIGATDMRPS |
| | -VL | | GTPARFSGSLLGGKAALTLSGVQPEDEAEYYCALWYSNRWVFGGGTKLTVL |
| 702 | E2M_LC_A107L- | artificial | GYAMN |
| | HCDR1 | | |
| 703 | E2M_LC_A107L- | artificial | RIRSKYNNYATYYADSVKE |
| | HCDR2 | | |
| 704 | E2M_LC_A107L- | artificial | HRNFGNSYLSWFAY |
| | HCDR3 | | |
| 705 | E2M_LC_A107L- | artificial | RSSTGAVTSGYYPN |
| | LCDR1 | | |
| 706 | E2M_LC_A107L- | artificial | ATDMRPS |
| | LCDR2 | | |
| 707 | E2M_LC_A107L- | artificial | LLWYSNRWV |
| | LCDR3 | | |
| 708 | E2M_LC_A107L- | artificial | EVQLVESGGGLVQPGGSLKLSCAASGFTFNGYAMNWVRQAPGKGLEWVARIRSKYNNY |
| | VH | | ATYYADSVKERFTISRDDSKNTAYLQMNNLKTEDTAVYYCVRHRNFGNSYLSWFAYWG |
| | | | QGTLVTVSS |
| 709 | E2M_LC_A107L- | artificial | QTVVTQEPSLTVSPGGTVTLTCRSSTGAVTSGYYPNWVQQKPGQAPRGLIGATDMRPS |
| | VL | | GTPARFSGSLLGGKAALTLSGVQPEDEAEYYCLLWYSNRWVFGGGTKLTVL |
| 710 | E2M_LC_V44I | artificial | EVQLVESGGGLVQPGGSLKLSCAASGFTFNGYAMNWVRQAPGKGLEWVARIRSKYNNY |
| | -VH | | ATYYADSVKERFTISRDDSKNTAYLQMNNLKTEDTAVYYCVRHRNFGNSYLSWFAYWG |
| | | | QGTLVTVSS |
| 711 | E2M_LC_V44I | artificial | QTVVTQEPSLTVSPGGTVTLTCRSSTGAVTSGYYPNWIQQKPGQAPRGLIGATDMRPS |
| | -VL | | GTPARFSGSLLGGKAALTLSGVQPEDEAEYYCALWYSNRWVFGGGTKLTVL |
| 712 | E2M_LC_V44L | artificial | EVQLVESGGGLVQPGGSLKLSCAASGFTFNGYAMNWVRQAPGKGLEWVARIRSKYNNY |
| | -VH | | ATYYADSVKERFTISRDDSKNTAYLQMNNLKTEDTAVYYCVRHRNFGNSYLSWFAYWG |
| | | | QGTLVTVSS |
| 713 | E2M_LC_V44L | artificial | QTVVTQEPSLTVSPGGTVTLTCRSSTGAVTSGYYPNWLQQKPGQAPRGLIGATDMRPS |
| | -VL | | GTPARFSGSLLGGKAALTLSGVQPEDEAEYYCALWYSNRWVFGGGTKLTVL |
| 714 | E2M_LC_V44M | artificial | EVQLVESGGGLVQPGGSLKLSCAASGFTFNGYAMNWVRQAPGKGLEWVARIRSKYNNY |
| | -VH | | ATYYADSVKERFTISRDDSKNTAYLQMNNLKTEDTAVYYCVRHRNFGNSYLSWFAYWG |
| | | | QGTLVTVSS |
| 715 | E2M_LC_V44M | artificial | QTVVTQEPSLTVSPGGTVTLTCRSSTGAVTSGYYPNWMQQKPGQAPRGLIGATDMRPS |
| | -VL | | GTPARFSGSLLGGKAALTLSGVQPEDEAEYYCALWYSNRWVFGGGTKLTVL |
| 716 | E2M_LC_W109Y- | artificial | GYAMN |
| | HCDR1 | | |
| 717 | E2M_LC_W109Y- | artificial | RIRSKYNNYATYYADSVKE |
| | HCDR2 | | |
| 718 | E2M_LC_W109Y- | artificial | HRNFGNSYLSWFAY |
| | HCDR3 | | |
| 719 | E2M_LC_W109Y- | artificial | RSSTGAVTSGYYPN |
| | LCDR1 | | |
| 720 | E2M_LC_W109Y- | artificial | ATDMRPS |
| | LCDR2 | | |
| 721 | E2M_LC_W109Y- | artificial | ALYYSNRWV |
| | LCDR3 | | |
| 722 | E2M_LC_W109Y- | artificial | EVQLVESGGGLVQPGGSLKLSCAASGFTENGYAMNWVRQAPGKGLEWVARIRSKYNNY |
| | VH | | ATYYADSVKERFTISRDDSKNTAYLQMNNLKTEDTAVYYCVRHRNFGNSYLSWFAYWG |
| | | | QGTLVTVSS |
| 723 | E2M_LC_W109Y- | artificial | QTVVTQEPSLTVSPGGTVTLTCRSSTGAVTSGYYPNWVQQKPGQAPRGLIGATDMRPS |
| | VL | | GTPARFSGSLLGGKAALTLSGVQPEDEAEYYCALYYSNRWVFGGGTKLTVL |
| 724 | F12Q- | artificial | SYAMN |
| | HCDR1 | | |
| 725 | F12Q- | artificial | RIRSKYNNYATYYADSVKG |
| | HCDR2 | | |
| 726 | F12Q- | artificial | HGNFGNSYVSWWAY |
| | HCDR3 | | |
| 727 | F12Q- | artificial | GSSTGAVTSGNYPN |
| | LCDR1 | | |
| 728 | F12Q | artificial | GTKFLAP |
| | LCDR2 | | |
| 729 | F12Q | artificial | VLWYSNRWV |
| | LCDR3 | | |
| 730 | F12Q-VH | artificial | EVQLVESGGGLVQPGGSLKLSCAASGFTFNSYAMNWVRQAPGKGLEWVARIRSKYNNY |
| | | | ATYYADSVKGRFTISRDDSKNTAYLQMNNLKTEDTAVYYCVRHGNFGNSYVSWWAYWG |
| | | | QGTLVTVSS |
| 731 | F12Q-VL | artificial | QTVVTQEPSLTVSPGGTVTLTCGSSTGAVTSGNYPNWVQQKPGQAPRGLIGGTKFLAP |
| | | | GTPARFSGSLLGGKAALTLSGVQPEDEAEYYCVLWYSNRWVFGGGTKLTVL |
| 732 | F12Q_HC_A89I- | artificial | EVQLVESGGGLVQPGGSLKLSCAASGFTFNSYAMNWVRQAPGKGLEWVARIRSKYNNY |
| | VH | | ATYYADSVKGRFTISRDDSKNTIYLQMNNLKTEDTAVYYCVRHGNFGNSYVSWWAYWG |
| | | | QGTLVTVSS |
| 733 | F12Q_HC_A89I- | artificial | QTVVTQEPSLTVSPGGTVTLTCGSSTGAVTSGNYPNWVQQKPGQAPRGLIGGTKFLAP |
| | VL | | GTPARFSGSLLGGKAALTLSGVQPEDEAEYYCVLWYSNRWVFGGGTKLTVL |
| 734 | F12Q_HC_A89L- | artificial | EVQLVESGGGLVQPGGSLKLSCAASGFTFNSYAMNWVRQAPGKGLEWVARIRSKYNNY |
| | VH | | ATYYADSVKGRFTISRDDSKNTLYLQMNNLKTEDTAVYYCVRHGNFGNSYVSWWAYWG |
| | | | QGTLVTVSS |
| 735 | F12Q_HC_A89L- | artificial | QTVVTQEPSLTVSPGGTVTLTCGSSTGAVTSGNYPNWVQQKPGQAPRGLIGGTKFLAP |
| | VL | | GTPARFSGSLLGGKAALTLSGVQPEDEAEYYCVLWYSNRWVFGGGTKLTVL |
| 736 | F12Q_HC_A89T- | artificial | EVQLVESGGGLVQPGGSLKLSCAASGFTFNSYAMNWVRQAPGKGLEWVARIRSKYNNY |
| | VH | | ATYYADSVKGRFTISRDDSKNTTYLQMNNLKTEDTAVYYCVRHGNFGNSYVSWWAYWG |
| | | | QGTLVTVSS |
| 737 | F12Q_HC_A89T- | artificial | QTVVTQEPSLTVSPGGTVTLTCGSSTGAVTSGNYPNWVQQKPGQAPRGLIGGTKFLAP |
| | VL | | GTPARFSGSLLGGKAALTLSGVQPEDEAEYYCVLWYSNRWVFGGGTKLTVL |
| 738 | F12Q_HC_A89V- | artificial | EVQLVESGGGLVQPGGSLKLSCAASGFTENSYAMNWVRQAPGKGLEWVARIRSKYNNY |
| | VH | | ATYYADSVKGRFTISRDDSKNTVYLQMNNLKTEDTAVYYCVRHGNFGNSYVSWWAYWG |
| | | | QGTLVTVSS |
| 739 | F12Q_HC_A89V- | artificial | QTVVTQEPSLTVSPGGTVTLTCGSSTGAVTSGNYPNWVQQKPGQAPRGLIGGTKFLAP |
| | VL | | GTPARFSGSLLGGKAALTLSGVQPEDEAEYYCVLWYSNRWVFGGGTKLTVL |
| 740 | F12Q_HC_L52M- | artificial | EVQLVESGGGLVQPGGSLKLSCAASGFTFNSYAMNWVRQAPGKGMEWVARIRSKYNNY |
| | VH | | ATYYADSVKGRFTISRDDSKNTAYLQMNNLKTEDTAVYYCVRHGNFGNSYVSWWAYWG |
| | | | QGTLVTVSS |
| 741 | F12Q_HC_L52M- | artificial | QTVVTQEPSLTVSPGGTVTLTCGSSTGAVTSGNYPNWVQQKPGQAPRGLIGGTKFLAP |
| | VL | | GTPARFSGSLLGGKAALTLSGVQPEDEAEYYCVLWYSNRWVFGGGTKLTVL |
| 742 | F12Q_HC_L52M_ | artificial | EVQLVESGGGLVQPGGSLKLSCAASGFTFNSYAMNWVRQAPGKGMEWVARIRSKYNNY |
| | LC_L21I_ | | ATYYADSVKGRFTISRDDSKNTAYLQMNNLKTEDTAVYYCVRHGNFGNSYVSWWAYWG |
| | Q46K_G141S- | | QGTLVTVSS |
| | VH | | |
| 743 | F12Q_HC_L52M_ | artificial | QTVVTQEPSLTVSPGGTVTITCGSSTGAVTSGNYPNWVQKKPGQAPRGLIGGTKFLAP |
| | LC_L21I_ | | GTPARFSGSLLGGKAALTLSGVQPEDEAEYYCVLWYSNRWVFGSGTKLTVL |
| | Q46K_G141S- | | |
| | VL | | |
| 744 | F12Q_HC_L52M_ | artificial | EVQLVESGGGLVQPGGSLKLSCAASGFTENSYAMNWVRQAPGKGMEWVARIRSKYNNY |
| | LC_Q46K- | | ATYYADSVKGRFTISRDDSKNTAYLQMNNLKTEDTAVYYCVRHGNFGNSYVSWWAYWG |
| | VH | | QGTLVTVSS |
| 745 | F12Q_HC_L52M_ | artificial | QTVVTQEPSLTVSPGGTVTLTCGSSTGAVTSGNYPNWVQKKPGQAPRGLIGGTKFLAP |
| | LC_Q46K- | | GTPARFSGSLLGGKAALTLSGVQPEDEAEYYCVLWYSNRWVFGGGTKLTVL |
| | VL | | |
| 746 | F12Q_HC_L52M_ | artificial | EVQLVESGGGLVQPGGSLKLSCAASGFTFNSYAMNWVRQAPGKGMEWVARIRSKYNNY |
| | LC_Q46K_ | | ATYYADSVKGRFTISRDDSKNTAYLQMNNLKTEDTAVYYCVRHGNFGNSYVSWWAYWG |
| | G141S-VH | | QGTLVTVSS |
| 747 | F12Q_HC_L52M_ | artificial | QTVVTQEPSLTVSPGGTVTLTCGSSTGAVTSGNYPNWVQKKPGQAPRGLIGGTKFLAP |
| | LC_Q46K_ | | GTPARFSGSLLGGKAALTLSGVQPEDEAEYYCVLWYSNRWVFGSGTKLTVL |
| | G141S-VL | | |
| 748 | F12Q_HC_L52V- | artificial | EVQLVESGGGLVQPGGSLKLSCAASGFTFNSYAMNWVRQAPGKGVEWVARIRSKYNNY |
| | VH | | ATYYADSVKGRFTISRDDSKNTAYLQMNNLKTEDTAVYYCVRHGNFGNSYVSWWAYWG |
| | | | QGTLVTVSS |
| 749 | F12Q_HC_L52V- | artificial | QTVVTQEPSLTVSPGGTVTLTCGSSTGAVTSGNYPNWVQQKPGQAPRGLIGGTKFLAP |
| | VL | | GTPARFSGSLLGGKAALTLSGVQPEDEAEYYCVLWYSNRWVFGGGTKLTVL |
| 750 | F12Q_HC_L52V_ | artificial | EVQLVESGGGLVQPGGSLKLSCAASGFTFNSYAMNWVRQAPGKGVEWVARIRSKYNNY |
| | LC_V44F- | | ATYYADSVKGRFTISRDDSKNTAYLQMNNLKTEDTAVYYCVRHGNFGNSYVSWWAYWG |
| | VH | | QGTLVTVSS |
| 751 | F12Q_HC_L52V_ | artificial | QTVVTQEPSLTVSPGGTVTLTCGSSTGAVTSGNYPNWFQQKPGQAPRGLIGGTKFLAP |
| | LC_V44F- | | GTPARFSGSLLGGKAALTLSGVQPEDEAEYYCVLWYSNRWVFGGGTKLTVL |
| | VL | | |
| 752 | F12Q_HC_L52V_ | artificial | EVQLVESGGGLVQPGGSLKLSCAASGFTFNSYAMNWVRQAPGKGVEWVARIRSKYNNY |
| | LC_V44Y- | | ATYYADSVKGRFTISRDDSKNTAYLQMNNLKTEDTAVYYCVRHGNFGNSYVSWWAYWG |
| | VH | | QGTLVTVSS |
| 753 | F12Q_HC_L52V_ | artificial | QTVVTQEPSLTVSPGGTVTLTCGSSTGAVTSGNYPNWYQQKPGQAPRGLIGGTKFLAP |
| | LC_V44Y- | | GTPARFSGSLLGGKAALTLSGVQPEDEAEYYCVLWYSNRWVFGGGTKLTVL |
| | VL | | |
| 754 | F12Q_HC_M41F_ | artificial | SYAFN |
| | A89V- | | |
| | HCDR1 | | |
| 755 | F12Q_HC_M41F_ | artificial | RIRSKYNNYATYYADSVKG |
| | A89V-HCDR2 | | |
| 756 | F12Q_HC_M41F_ | artificial | HGNFGNSYVSWWAY |
| | A89V-HCDR3 | | |
| 757 | F12Q_HC_M41F_ | artificial | GSSTGAVTSGNYPN |
| | A89V- | | |
| | LCDR1 | | |
| 758 | F12Q_HC_M41F_ | artificial | GTKFLAP |
| | A89V- | | |
| | LCDR2 | | |
| 759 | F12Q_HC_M41F_ | artificial | VLWYSNRWV |
| | A89V- | | |
| | LCDR3 | | |
| 760 | F12Q_HC_M41F_ | artificial | EVQLVESGGGLVQPGGSLKLSCAASGFTENSYAFNWVRQAPGKGLEWVARIRSKYNNY |
| | A89V-VH | | ATYYADSVKGRFTISRDDSKNTVYLQMNNLKTEDTAVYYCVRHGNFGNSYVSWWAYWG |
| | | | QGTLVTVSS |
| 761 | F12Q_HC_M41F_ | artificial | QTVVTQEPSLTVSPGGTVTLTCGSSTGAVTSGNYPNWVQQKPGQAPRGLIGGTKFLAP |
| | A89V-VL | | GTPARFSGSLLGGKAALTLSGVQPEDEAEYYCVLWYSNRWVFGGGTKLTVL |
| 762 | F12Q_HC_M41I- | artificial | SYAIN |
| | HCDR1 | | |
| 763 | F12Q_HC_M41I- | artificial | RIRSKYNNYATYYADSVKG |
| | HCDR2 | | |
| 764 | F12Q_HC_M41I- | artificial | HGNFGNSYVSWWAY |
| | HCDR3 | | |
| 765 | F12Q_HC_M41I- | artificial | GSSTGAVTSGNYPN |
| | LCDR1 | | |
| 766 | F12Q_HC_M41I- | artificial | GTKFLAP |
| | LCDR2 | | |
| 767 | F12Q_HC_M41I- | artificial | VLWYSNRWV |
| | LCDR3 | | |
| 768 | F12Q_HC_M41I- | artificial | EVQLVESGGGLVQPGGSLKLSCAASGFTFNSYAINWVRQAPGKGLEWVARIRSKYNNY |
| | VH | | ATYYADSVKGRFTISRDDSKNTAYLQMNNLKTEDTAVYYCVRHGNFGNSYVSWWAYWG |
| | | | QGTLVTVSS |
| 769 | F12Q_HC_M41I- | artificial | QTVVTQEPSLTVSPGGTVTLTCGSSTGAVTSGNYPNWVQQKPGQAPRGLIGGTKFLAP |
| | VL | | GTPARFSGSLLGGKAALTLSGVQPEDEAEYYCVLWYSNRWVFGGGTKLTVL |
| 770 | F12Q_HC_M41I_ | artificial | EVQLVESGGGLVQPGGSLKLSCAASGFTENSYAINWVRQAPGKGLEWVARIRSKYNNY |
| | A89L-VH | | ATYYADSVKGRFTISRDDSKNTLYLQMNNLKTEDTAVYYCVRHGNFGNSYVSWWAYWG |
| | | | QGTLVTVSS |
| 771 | F12Q_HC_M41I_ | artificial | QTVVTQEPSLTVSPGGTVTLTCGSSTGAVTSGNYPNWVQQKPGQAPRGLIGGTKFLAP |
| | A89L-VL | | GTPARFSGSLLGGKAALTLSGVQPEDEAEYYCVLWYSNRWVFGGGTKLTVL |
| 772 | F12Q_HC_M41I_ | artificial | EVQLVESGGGLVQPGGSLKLSCAASGFTENSYAINWVRQAPGKGLEWVARIRSKYNNY |
| | A89V-VH | | ATYYADSVKGRFTISRDDSKNTVYLQMNNLKTEDTAVYYCVRHGNFGNSYVSWWAYWG |
| | | | QGTLVTVSS |
| 773 | F12Q_HC_M41I_ | artificial | QTVVTQEPSLTVSPGGTVTLTCGSSTGAVTSGNYPNWVQQKPGQAPRGLIGGTKFLAP |
| | A89V-VL | | GTPARFSGSLLGGKAALTLSGVQPEDEAEYYCVLWYSNRWVFGGGTKLTVL |
| 774 | F12Q_HC_M41I_ | artificial | EVQLVESGGGLVQPGGSLKLSCAASGFTFNSYAINWVRQAPGKGLEWVARIRSKYNNY |
| | A89V_LC_ | | ATYYADSVKGRFTISRDDSKNTVYLQMNNLKTEDTAVYYCVRHGNFGNSYVSWWAYWG |
| | L21I_Q46K_ | | QGTLVTVSS |
| | 4G11S-VH | | |
| 775 | F12Q_HC_M41I_ | artificial | QTVVTQEPSLTVSPGGTVTITCGSSTGAVTSGNYPNWVQKKPGQAPRGLIGGTKFLAP |
| | A89V_LC_ | | GTPARFSGSLLGGKAALTLSGVQPEDEAEYYCVLWYSNRWVFGSGTKLTVL |
| | L21I_Q46K_ | | |
| | G141S-VL | | |
| 776 | F12Q_HC_M41I_ | artificial | EVQLVESGGGLVQPGGSLKLSCAASGFTFNSYAINWVRQAPGKGLEWVARIRSKYNNY |
| | A89V_LC_ | | ATYYADSVKGRFTISRDDSKNTVYLQMNNLKTEDTAVYYCVRHGNFGNSYVSWWAYWG |
| | Q46K-VH | | QGTLVTVSS |
| 777 | F12Q_HC_M41I_ | artificial | QTVVTQEPSLTVSPGGTVTLTCGSSTGAVTSGNYPNWVQKKPGQAPRGLIGGTKFLAP |
| | A89V_LC_ | | GTPARFSGSLLGGKAALTLSGVQPEDEAEYYCVLWYSNRWVFGGGTKLTVL |
| | Q46K-VL | | |
| 778 | F12Q_HC_M41I_ | artificial | EVQLVESGGGLVQPGGSLKLSCAASGFTFNSYAINWVRQAPGKGVEWVARIRSKYNNY |
| | L52V_A89V_ | | ATYYADSVKGRFTISRDDSKNTVYLQMNNLKTEDTAVYYCVRHGNFGNSYVSWWAYWG |
| | LC_L21I_ | | QGTLVTVSS |
| | Q46K_G141S_ | | |
| | V44F-VH | | |
| 779 | F12Q_HC_M41I_ | artificial | QTVVTQEPSLTVSPGGTVTITCGSSTGAVTSGNYPNWFQKKPGQAPRGLIGGTKFLAP |
| | L52V_A89V_ | | GTPARFSGSLLGGKAALTLSGVQPEDEAEYYCVLWYSNRWVFGSGTKLTVL |
| | LC_L21I_ | | |
| | Q46K_G141S_ | | |
| | V44F-VL | | |
| 780 | F12Q_HC_M41I_ | artificial | EVQLVESGGGLVQPGGSLKLSCAASGFTFNSYAINWVRQAPGKGLEWVARIRSKYNNY |
| | LC_L21I_ | | ATYYADSVKGRFTISRDDSKNTAYLQMNNLKTEDTAVYYCVRHGNFGNSYVSWWAYWG |
| | Q46K_G141S- | | QGTLVTVSS |
| | VH | | |
| 781 | F12Q_HC_M41I_ | artificial | QTVVTQEPSLTVSPGGTVTITCGSSTGAVTSGNYPNWVQKKPGQAPRGLIGGTKFLAP |
| | LC_L21I_ | | GTPARFSGSLLGGKAALTLSGVQPEDEAEYYCVLWYSNRWVFGSGTKLTVL |
| | Q46K_G141S- | | |
| | VL | | |
| 782 | F12Q_HC_M41I_ | artificial | EVQLVESGGGLVQPGGSLKLSCAASGFTFNSYAINWVREAPGKGLEWVARIRSKYNNY |
| | Q46E_A89V_ | | ATYYADSVKGRFTISRDDSKNTVYLQMNNLKTEDTAVYYCVRHGNFGNSYVSWWAYWG |
| | LC_L21I_ | | QGTLVTVSS |
| | Q46K_G141S- | | |
| | VH | | |
| 783 | F12Q_HC_M41I_ | artificial | QTVVTQEPSLTVSPGGTVTITCGSSTGAVTSGNYPNWVQKKPGQAPRGLIGGTKFLAP |
| | Q46E_A89V_ | | GTPARFSGSLLGGKAALTLSGVQPEDEAEYYCVLWYSNRWVFGSGTKLTVL |
| | LC_L21I_ | | |
| | Q46K_G141S- | | |
| | VL | | |
| 784 | F12Q_HC_M41V- | artificial | SYAVN |
| | HCDR1 | | |
| 785 | F12Q_HC_M41V- | artificial | RIRSKYNNYATYYADSVKG |
| | HCDR2 | | |
| 786 | F12Q_HC_M41V- | artificial | HGNFGNSYVSWWAY |
| | HCDR3 | | |
| 787 | F12Q_HC_M41V- | artificial | GSSTGAVTSGNYPN |
| | LCDR1 | | |
| 788 | F12Q_HC_M41V- | artificial | GTKFLAP |
| | LCDR2 | | |
| 789 | F12Q_HC_M41V- | artificial | VLWYSNRWV |
| | LCDR3 | | |
| 790 | F12Q_HC_M41V- | artificial | EVQLVESGGGLVQPGGSLKLSCAASGFTFNSYAVNWVRQAPGKGLEWVARIRSKYNNY |
| | VH | | ATYYADSVKGRFTISRDDSKNTAYLQMNNLKTEDTAVYYCVRHGNFGNSYVSWWAYWG |
| | | | QGTLVTVSS |
| 791 | F12Q_HC_M41V- | artificial | QTVVTQEPSLTVSPGGTVTLTCGSSTGAVTSGNYPNWVQQKPGQAPRGLIGGTKFLAP |
| | VL | | GTPARFSGSLLGGKAALTLSGVQPEDEAEYYCVLWYSNRWVFGGGTKLTVL |
| 792 | F12Q_HC_Q46D- | artificial | EVQLVESGGGLVQPGGSLKLSCAASGFTFNSYAMNWVRDAPGKGLEWVARIRSKYNNY |
| | VH | | ATYYADSVKGRFTISRDDSKNTAYLQMNNLKTEDTAVYYCVRHGNFGNSYVSWWAYWG |
| | | | QGTLVTVSS |
| 793 | F12Q_HC_Q46D- | artificial | QTVVTQEPSLTVSPGGTVTLTCGSSTGAVTSGNYPNWVQQKPGQAPRGLIGGTKFLAP |
| | VL | | GTPARFSGSLLGGKAALTLSGVQPEDEAEYYCVLWYSNRWVFGGGTKLTVL |
| 794 | F12Q_HC_Q46D_ | artificial | EVQLVESGGGLVQPGGSLKLSCAASGFTFNSYAMNWVRDAPGKGLEWVARIRSKYNNY |
| | LC_L21I_ | | ATYYADSVKGRFTISRDDSKNTAYLQMNNLKTEDTAVYYCVRHGNFGNSYVSWWAYWG |
| | Q46K_G141S- | | QGTLVTVSS |
| | VH | | |
| 795 | F12Q_HC_Q46D_ | artificial | QTVVTQEPSLTVSPGGTVTITCGSSTGAVTSGNYPNWVQKKPGQAPRGLIGGTKFLAP |
| | LC_L211_ | | GTPARFSGSLLGGKAALTLSGVQPEDEAEYYCVLWYSNRWVFGSGTKLTVL |
| | Q46K_G141S- | | |
| | VL | | |
| 796 | F12Q_HC_Q46D_ | artificial | EVQLVESGGGLVQPGGSLKLSCAASGFTFNSYAMNWVRDAPGKGLEWVARIRSKYNNY |
| | LC_L21I_ | | ATYYADSVKGRFTISRDDSKNTAYLQMNNLKTEDTAVYYCVRHGNFGNSYVSWWAYWG |
| | Q46R_G141S- | | QGTLVTVSS |
| | VH | | |
| 797 | F12Q_HC_Q46D_ | artificial | QTVVTQEPSLTVSPGGTVTITCGSSTGAVTSGNYPNWVQRKPGQAPRGLIGGTKFLAP |
| | LC_L21I_ | | GTPARFSGSLLGGKAALTLSGVQPEDEAEYYCVLWYSNRWVFGSGTKLTVL |
| | Q46R_G141S- | | |
| | VL | | |
| 798 | F12Q_HC_Q46D_ | artificial | EVQLVESGGGLVQPGGSLKLSCAASGFTFNSYAMNWVRDAPGKGLEWVARIRSKYNNY |
| | LC_Q46K- | | ATYYADSVKGRFTISRDDSKNTAYLQMNNLKTEDTAVYYCVRHGNFGNSYVSWWAYWG |
| | VH | | QGTLVTVSS |
| 799 | F12Q_HC_Q46D_ | artificial | QTVVTQEPSLTVSPGGTVTLTCGSSTGAVTSGNYPNWVQKKPGQAPRGLIGGTKFLAP |
| | LC_Q46K- | | GTPARFSGSLLGGKAALTLSGVQPEDEAEYYCVLWYSNRWVFGGGTKLTVL |
| | VL | | |
| 800 | F12Q_HC_Q46D_ | artificial | EVQLVESGGGLVQPGGSLKLSCAASGFTFNSYAMNWVRDAPGKGLEWVARIRSKYNNY |
| | LC_Q46K_ | | ATYYADSVKGRFTISRDDSKNTAYLQMNNLKTEDTAVYYCVRHGNFGNSYVSWWAYWG |
| | G141S-VH | | QGTLVTVSS |
| 801 | F12Q_HC_Q46D_ | artificial | QTVVTQEPSLTVSPGGTVTLTCGSSTGAVTSGNYPNWVQKKPGQAPRGLIGGTKFLAP |
| | LC_Q46K_ | | GTPARFSGSLLGGKAALTLSGVQPEDEAEYYCVLWYSNRWVFGSGTKLTVL |
| | G141S-VL | | |
| 802 | F12Q_HC_Q46D_ | artificial | EVQLVESGGGLVQPGGSLKLSCAASGFTFNSYAMNWVRDAPGKGLEWVARIRSKYNNY |
| | LC_Q46R- | | ATYYADSVKGRFTISRDDSKNTAYLQMNNLKTEDTAVYYCVRHGNFGNSYVSWWAYWG |
| | VH | | QGTLVTVSS |
| 803 | F12Q_HC_Q46D_ | artificial | QTVVTQEPSLTVSPGGTVTLTCGSSTGAVTSGNYPNWVQRKPGQAPRGLIGGTKFLAP |
| | LC_Q46R- | | GTPARFSGSLLGGKAALTLSGVQPEDEAEYYCVLWYSNRWVFGGGTKLTVL |
| | VL | | |
| 804 | F12Q_HC_Q46D_ | artificial | EVQLVESGGGLVQPGGSLKLSCAASGFTFNSYAMNWVRDAPGKGLEWVARIRSKYNNY |
| | LC_Q46R_ | | ATYYADSVKGRFTISRDDSKNTAYLQMNNLKTEDTAVYYCVRHGNFGNSYVSWWAYWG |
| | G141S-VH | | QGTLVTVSS |
| 805 | F12Q_HC_Q46D_ | artificial | QTVVTQEPSLTVSPGGTVTLTCGSSTGAVTSGNYPNWVQRKPGQAPRGLIGGTKFLAP |
| | LC_Q46R_ | | GTPARFSGSLLGGKAALTLSGVQPEDEAEYYCVLWYSNRWVFGSGTKLTVL |
| | G141S-VL | | |
| 806 | F12Q_HC_Q46E- | artificial | EVQLVESGGGLVQPGGSLKLSCAASGFTFNSYAMNWVREAPGKGLEWVARIRSKYNNY |
| | VH | | ATYYADSVKGRFTISRDDSKNTAYLQMNNLKTEDTAVYYCVRHGNFGNSYVSWWAYWG |
| | | | QGTLVTVSS |
| 807 | F12Q_HC_Q46E- | artificial | QTVVTQEPSLTVSPGGTVTLTCGSSTGAVTSGNYPNWVQQKPGQAPRGLIGGTKFLAP |
| | VL | | GTPARFSGSLLGGKAALTLSGVQPEDEAEYYCVLWYSNRWVFGGGTKLTVL |
| 808 | F12Q_HC_Q46E_ | artificial | EVQLVESGGGLVQPGGSLKLSCAASGFTFNSYAMNWVREAPGKGLEWVARIRSKYNNY |
| | LC_L21I_ | | ATYYADSVKGRFTISRDDSKNTAYLQMNNLKTEDTAVYYCVRHGNFGNSYVSWWAYWG |
| | Q46K_G141S- | | QGTLVTVSS |
| | VH | | |
| 809 | F12Q_HC_Q46E_ | artificial | QTVVTQEPSLTVSPGGTVTITCGSSTGAVTSGNYPNWVQKKPGQAPRGLIGGTKFLAP |
| | LC_L21I_ | | GTPARFSGSLLGGKAALTLSGVQPEDEAEYYCVLWYSNRWVFGSGTKLTVL |
| | Q46K_G141S- | | |
| | VL | | |
| 810 | F12Q_HC_Q46E_ | artificial | EVQLVESGGGLVQPGGSLKLSCAASGFTFNSYAMNWVREAPGKGLEWVARIRSKYNNY |
| | LC_L21I_ | | ATYYADSVKGRFTISRDDSKNTAYLQMNNLKTEDTAVYYCVRHGNFGNSYVSWWAYWG |
| | Q46R_G141S- | | QGTLVTVSS |
| | VH | | |
| 811 | F12Q_HC_Q46E_ | artificial | QTVVTQEPSLTVSPGGTVTITCGSSTGAVTSGNYPNWVQRKPGQAPRGLIGGTKFLAP |
| | LC_L21I_ | | GTPARFSGSLLGGKAALTLSGVQPEDEAEYYCVLWYSNRWVFGSGTKLTVL |
| | Q46R_G141S- | | |
| | VL | | |
| 812 | F12Q_HC_Q46E_ | artificial | EVQLVESGGGLVQPGGSLKLSCAASGFTENSYAMNWVREAPGKGLEWVARIRSKYNNY |
| | LC_Q46K- | | ATYYADSVKGRFTISRDDSKNTAYLQMNNLKTEDTAVYYCVRHGNFGNSYVSWWAYWG |
| | VH | | QGTLVTVSS |
| 813 | F12Q_HC_Q46E_ | artificial | QTVVTQEPSLTVSPGGTVTLTCGSSTGAVTSGNYPNWVQKKPGQAPRGLIGGTKFLAP |
| | LC_Q46K- | | GTPARFSGSLLGGKAALTLSGVQPEDEAEYYCVLWYSNRWVFGGGTKLTVL |
| | VL | | |
| 814 | F12Q_HC_Q46E_ | artificial | EVQLVESGGGLVQPGGSLKLSCAASGFTENSYAMNWVREAPGKGLEWVARIRSKYNNY |
| | LC_Q46K_ | | ATYYADSVKGRFTISRDDSKNTAYLQMNNLKTEDTAVYYCVRHGNFGNSYVSWWAYWG |
| | G141S-VH | | QGTLVTVSS |
| 815 | F12Q_HC_Q46E_ | artificial | QTVVTQEPSLTVSPGGTVTLTCGSSTGAVTSGNYPNWVQKKPGQAPRGLIGGTKFLAP |
| | LC_Q46K_ | | GTPARFSGSLLGGKAALTLSGVQPEDEAEYYCVLWYSNRWVFGSGTKLTVL |
| | G141S-VL | | |
| 816 | F12Q_HC_Q46E_ | artificial | EVQLVESGGGLVQPGGSLKLSCAASGFTFNSYAMNWVREAPGKGLEWVARIRSKYNNY |
| | LC_Q46R- | | ATYYADSVKGRFTISRDDSKNTAYLQMNNLKTEDTAVYYCVRHGNFGNSYVSWWAYWG |
| | VH | | QGTLVTVSS |
| 817 | F12Q_HC_Q46E_ | artificial | QTVVTQEPSLTVSPGGTVTLTCGSSTGAVTSGNYPNWVQRKPGQAPRGLIGGTKFLAP |
| | LC_Q46R- | | GTPARFSGSLLGGKAALTLSGVQPEDEAEYYCVLWYSNRWVFGGGTKLTVL |
| | VL | | |
| 818 | F12Q_HC_Q46E_ | artificial | EVQLVESGGGLVQPGGSLKLSCAASGFTFNSYAMNWVREAPGKGLEWVARIRSKYNNY |
| | LC_Q46R_ | | ATYYADSVKGRFTISRDDSKNTAYLQMNNLKTEDTAVYYCVRHGNFGNSYVSWWAYWG |
| | G141S-VH | | QGTLVTVSS |
| 819 | F12Q_HC_Q46E_ | artificial | QTVVTQEPSLTVSPGGTVTLTCGSSTGAVTSGNYPNWVQRKPGQAPRGLIGGTKFLAP |
| | LC_Q46R_ | | GTPARFSGSLLGGKAALTLSGVQPEDEAEYYCVLWYSNRWVFGSGTKLTVL |
| | G141S-VL | | |
| 820 | F12Q_HC_Q46K- | artificial | EVQLVESGGGLVQPGGSLKLSCAASGFTFNSYAMNWVRKAPGKGLEWVARIRSKYNNY |
| | VH | | ATYYADSVKGRFTISRDDSKNTAYLQMNNLKTEDTAVYYCVRHGNFGNSYVSWWAYWG |
| | | | QGTLVTVSS |
| 821 | F12Q_HC_Q46K- | artificial | QTVVTQEPSLTVSPGGTVTLTCGSSTGAVTSGNYPNWVQQKPGQAPRGLIGGTKFLAP |
| | VL | | GTPARFSGSLLGGKAALTLSGVQPEDEAEYYCVLWYSNRWVFGGGTKLTVL |
| 822 | F12Q_HC_Q46R- | artificial | EVQLVESGGGLVQPGGSLKLSCAASGFTFNSYAMNWVRRAPGKGLEWVARIRSKYNNY |
| | VH | | ATYYADSVKGRFTISRDDSKNTAYLQMNNLKTEDTAVYYCVRHGNFGNSYVSWWAYWG |
| | | | QGTLVTVSS |
| 823 | F12Q_HC_Q46R- | artificial | QTVVTQEPSLTVSPGGTVTLTCGSSTGAVTSGNYPNWVQQKPGQAPRGLIGGTKFLAP |
| | VL | | GTPARFSGSLLGGKAALTLSGVQPEDEAEYYCVLWYSNRWVFGGGTKLTVL |
| 824 | F12Q_HC_V107A- | artificial | EVQLVESGGGLVQPGGSLKLSCAASGFTFNSYAMNWVRQAPGKGLEWVARIRSKYNNY |
| | VH | | ATYYADSVKGRFTISRDDSKNTAYLQMNNLKTEDTAVYYCARHGNFGNSYVSWWAYWG |
| | | | QGTLVTVSS |
| 825 | F12Q_HC_V107A- | artificial | QTVVTQEPSLTVSPGGTVTLTCGSSTGAVTSGNYPNWVQQKPGQAPRGLIGGTKFLAP |
| | VL | | GTPARFSGSLLGGKAALTLSGVQPEDEAEYYCVLWYSNRWVFGGGTKLTVL |
| 826 | F12Q_HC_V107L- | artificial | EVQLVESGGGLVQPGGSLKLSCAASGFTFNSYAMNWVRQAPGKGLEWVARIRSKYNNY |
| | VH | | ATYYADSVKGRFTISRDDSKNTAYLQMNNLKTEDTAVYYCLRHGNFGNSYVSWWAYWG |
| | | | QGTLVTVSS |
| 827 | F12Q_HC_V107L- | artificial | QTVVTQEPSLTVSPGGTVTLTCGSSTGAVTSGNYPNWVQQKPGQAPRGLIGGTKFLAP |
| | VL | | GTPARFSGSLLGGKAALTLSGVQPEDEAEYYCVLWYSNRWVFGGGTKLTVL |
| 828 | F12Q_HC_W136F- | artificial | SYAMN |
| | HCDR1 | | |
| 829 | F12Q_HC_W136F- | artificial | RIRSKYNNYATYYADSVKG |
| | HCDR2 | | |
| 830 | F12Q_HC_W136F- | artificial | HGNFGNSYVSWFAY |
| | HCDR3 | | |
| 831 | F12Q_HC_W136F- | artificial | GSSTGAVTSGNYPN |
| | LCDR1 | | |
| 832 | F12Q_HC_W136F- | artificial | GTKFLAP |
| | LCDR2 | | |
| 833 | F12Q_HC_W136F- | artificial | VLWYSNRWV |
| | LCDR3 | | |
| 834 | F12Q_HC_W136F- | artificial | EVQLVESGGGLVQPGGSLKLSCAASGFTENSYAMNWVRQAPGKGLEWVARIRSKYNNY |
| | VH | | ATYYADSVKGRFTISRDDSKNTAYLQMNNLKTEDTAVYYCVRHGNFGNSYVSWFAYWG |
| | | | QGTLVTVSS |
| 835 | F12Q_HC_W136F- | artificial | QTVVTQEPSLTVSPGGTVTLTCGSSTGAVTSGNYPNWVQQKPGQAPRGLIGGTKFLAP |
| | VL | | GTPARFSGSLLGGKAALTLSGVQPEDEAEYYCVLWYSNRWVFGGGTKLTVL |
| 836 | F12Q_HC_W136F_ | artificial | EVQLVESGGGLVQPGGSLKLSCAASGFTFNSYAMNWVRQAPGKGLEWVARIRSKYNNY |
| | LC_V44I | | ATYYADSVKGRFTISRDDSKNTAYLQMNNLKTEDTAVYYCVRHGNFGNSYVSWFAYWG |
| | -VH | | QGTLVTVSS |
| 837 | F12Q_HC_W136F_ | artificial | QTVVTQEPSLTVSPGGTVTLTCGSSTGAVTSGNYPNWIQQKPGQAPRGLIGGTKFLAP |
| | LC_V44I | | GTPARFSGSLLGGKAALTLSGVQPEDEAEYYCVLWYSNRWVFGGGTKLTVL |
| | -VL | | |
| 838 | F12Q_HC_W136F_ | artificial | EVQLVESGGGLVQPGGSLKLSCAASGFTENSYAMNWVRQAPGKGLEWVARIRSKYNNY |
| | LC_V44L | | ATYYADSVKGRFTISRDDSKNTAYLQMNNLKTEDTAVYYCVRHGNFGNSYVSWFAYWG |
| | -VH | | QGTLVTVSS |
| 839 | F12Q_HC_W136F_ | artificial | QTVVTQEPSLTVSPGGTVTLTCGSSTGAVTSGNYPNWLQQKPGQAPRGLIGGTKFLAP |
| | LC_V44L | | GTPARFSGSLLGGKAALTLSGVQPEDEAEYYCVLWYSNRWVFGGGTKLTVL |
| | -VL | | |
| 840 | F12Q_I2E- | artificial | SYAIN |
| | HCDR1 | | |
| 841 | F12Q_I2E- | artificial | RIRSKYNNYATYYADAVKG |
| | HCDR2 | | |
| 842 | F12Q_I2E- | artificial | AGNFGSSYVSWWAY |
| | HCDR3 | | |
| 843 | F12Q_I2E- | artificial | GSSTGAVTSGNYPN |
| | LCDR1 | | |
| 844 | F12Q_I2E- | artificial | GTKFLAP |
| | LCDR2 | | |
| 845 | F12Q_I2E- | artificial | VLWYSNRWV |
| | LCDR3 | | |
| 846 | F12Q_I2E- | artificial | EVQLVESGGGLVQPGGSLKLSCAASGFTFNSYAINWVRQAPGKGLEWVARIRSKYNNY |
| | VH | | ATYYADAVKGRFTISRDDSKNTVYLQMNNLKTEDTAVYYCARAGNFGSSYVSWWAYWG |
| | | | QGTLVTVSS |
| 847 | F12Q_I2E- | artificial | QTVVTQEPSLTVSPGGTVTITCGSSTGAVTSGNYPNWVQKKPGQAPRGLIGGTKFLAP |
| | VL | | GTPARFSGSLSGGKAALTLSGVQPEDEAEYYCVLWYSNRWVFGSGTKLTVL |
| 848 | F12Q_I2K- | artificial | SYAMN |
| | HCDR1 | | |
| 849 | F12Q_I2K- | artificial | RIRSKYNNYATYYAEAVKG |
| | HCDR2 | | |
| 850 | F12Q_I2K- | artificial | NENIGTSYVSWWAY |
| | HCDR3 | | |
| 851 | F12Q_I2K- | artificial | GSSTGAVTSGNYPN |
| | LCDR1 | | |
| 852 | F12Q_I2K- | artificial | GTKFLAP |
| | LCDR2 | | |
| 853 | F12Q_I2K- | artificial | VLWYSNRWV |
| | LCDR3 | | |
| 854 | F12Q_I2K- | artificial | EVQLVESGGGLVQPGGSLKLSCAASGFTFNSYAMNWVRQAPGKGLEWVARIRSKYNNY |
| | VH | | ATYYAEAVKGRFTISRDDSKNTVYLQMNNLKTEDTAVYYCVRNENIGTSYVSWWAYWG |
| | | | QGTLVTVSS |
| 855 | F12Q_I2K- | artificial | QTVVTQEPSLTVSPGGTVTITCGSSTGAVTSGNYPNWVQQKPGQAPRGLIGGTKFLAP |
| | VL | | GTPARFSGSLSGGKAALTLSGVQPEDEAEYYCVLWYSNRWVFGSGTKLTVL |
| 856 | F12Q_I2L- | artificial | SYAMN |
| | HCDR1 | | |
| 857 | F12Q_I2L- | artificial | RIRSKYNNYATYYADAVKG |
| | HCDR2 | | |
| 858 | F12Q_I2L- | artificial | AGNFGSSYVSWFAY |
| | HCDR3 | | |
| 859 | F12Q_I2L- | artificial | GSSTGAVTSGNYPN |
| | LCDR1 | | |
| 860 | F12Q_I2L- | artificial | GTKFLAP |
| | LCDR2 | | |
| 861 | F12Q_I2L- | artificial | VLYYSNRWV |
| | LCDR3 | | |
| 862 | F12Q_I2L- | artificial | EVQLVESGGGLVQPGGSLKLSCAASGFTFNSYAMNWVRQAPGKGMEWVARIRSKYNNY |
| | VH | | ATYYADAVKGRFTISRDDSKNTLYLQMNNLKTEDTAVYYCVRAGNFGSSYVSWFAYWG |
| | | | QGTLVTVSS |
| 863 | F12Q_I2L- | artificial | QTVVTQEPSLTVSPGGTVTITCGSSTGAVTSGNYPNWIQKKPGQAPRGLIGGTKFLAP |
| | VL | | GTPARFSGSLEGGKAALTLSGVQPEDEAEYYCVLYYSNRWVFGSGTKLTVL |
| 864 | F12Q_I2M- | artificial | SYAMN |
| | HCDR1 | | |
| 865 | F12Q_I2M- | artificial | RIRSKYNNYATYYADAVKG |
| | HCDR2 | | |
| 866 | F12Q_I2M- | artificial | AGNFGTSYVSWWAY |
| | HCDR3 | | |
| 867 | F12Q_I2M- | artificial | GSSTGAVTSGNYPN |
| | LCDR1 | | |
| 868 | F12Q_I2M- | artificial | GTKFLAP |
| | LCDR2 | | |
| 869 | F12Q_I2M- | artificial | VLWYSNRWV |
| | LCDR3 | | |
| 870 | F12Q_I2M- | artificial | EVQLVESGGGLVQPGGSLKLSCAASGFTFNSYAMNWVRQAPGKGMEWVARIRSKYNNY |
| | VH | | ATYYADAVKGRFTISRDDSKNTLYLQMNNLKTEDTAVYYCVRAGNFGTSYVSWWAYWG |
| | | | QGTLVTVSS |
| 871 | F12Q_I2M- | artificial | QTVVTQEPSLTVSPGGTVTITCGSSTGAVTSGNYPNWVQKKPGQAPRGLIGGTKFLAP |
| | VL | | GTPARFSGSLSGGKAALTLSGVQPEDEAEYYCVLWYSNRWVFGSGTKLTVL |
| 872 | F12Q_I2M2- | artificial | SYAIN |
| | HCDR1 | | |
| 873 | F12Q_I2M2- | artificial | RIRSKYNNYATYYADAVKG |
| | HCDR2 | | |
| 874 | F12Q_I2M2- | artificial | NANFGTSYVSWFAY |
| | HCDR3 | | |
| 875 | F12Q_I2M2- | artificial | GSSTGAVTSGNYPN |
| | LCDR1 | | |
| 876 | F12Q_I2M2- | artificial | GTKFLAP |
| | LCDR2 | | |
| 877 | F12Q_I2M2- | artificial | VLWYSNRWV |
| | LCDR3 | | |
| 878 | F12Q_I2M2- | artificial | EVQLVESGGGLVQPGGSLKLSCAASGFTENSYAINWVREAPGKGLEWVARIRSKYNNY |
| | VH | | ATYYADAVKGRFTISRDDSKNTAYLQMNNLKTEDTAVYYCVRNANFGTSYVSWFAYWG |
| | | | QGTLVTVSS |
| 879 | F12Q_I2M2- | artificial | QTVVTQEPSLTVSPGGTVTLTCGSSTGAVTSGNYPNWVQKKPGQAPRGLIGGTKFLAP |
| | VL | | GTPARFSGSLLGGKAALTLSGVQPEDEAEYYCVLWYSNRWVFGSGTKLTVL |
| 880 | F12Q_LC_G141S- | artificial | EVQLVESGGGLVQPGGSLKLSCAASGFTFNSYAMNWVRQAPGKGLEWVARIRSKYNNY |
| | VH | | ATYYADSVKGRFTISRDDSKNTAYLQMNNLKTEDTAVYYCVRHGNFGNSYVSWWAYWG |
| | | | QGTLVTVSS |
| 881 | F12Q_LC_G141S- | artificial | QTVVTQEPSLTVSPGGTVTLTCGSSTGAVTSGNYPNWVQQKPGQAPRGLIGGTKFLAP |
| | VL | | GTPARFSGSLLGGKAALTLSGVQPEDEAEYYCVLWYSNRWVFGSGTKLTVL |
| 882 | F12Q_LC_G141S_ | artificial | EVQLVESGGGLVQPGGSLKLSCAASGFTFNSYAMNWVRQAPGKGLEWVARIRSKYNNY |
| | L21I- | | ATYYADSVKGRFTISRDDSKNTAYLQMNNLKTEDTAVYYCVRHGNFGNSYVSWWAYWG |
| | VH | | QGTLVTVSS |
| 883 | F12Q_LC_G141S_ | artificial | QTVVTQEPSLTVSPGGTVTITCGSSTGAVTSGNYPNWVQQKPGQAPRGLIGGTKFLAP |
| | L21I- | | GTPARFSGSLLGGKAALTLSGVQPEDEAEYYCVLWYSNRWVFGSGTKLTVL |
| | VL | | |
| 884 | F12Q_LC_L83E- | artificial | EVQLVESGGGLVQPGGSLKLSCAASGFTFNSYAMNWVRQAPGKGLEWVARIRSKYNNY |
| | VH | | ATYYADSVKGRFTISRDDSKNTAYLQMNNLKTEDTAVYYCVRHGNFGNSYVSWWAYWG |
| | | | QGTLVTVSS |
| 885 | F12Q_LC_L83E- | artificial | QTVVTQEPSLTVSPGGTVTLTCGSSTGAVTSGNYPNWVQQKPGQAPRGLIGGTKFLAP |
| | VL | | GTPARFSGSLEGGKAALTLSGVQPEDEAEYYCVLWYSNRWVFGGGTKLTVL |
| 886 | F12Q_LC_L83S- | artificial | EVQLVESGGGLVQPGGSLKLSCAASGFTENSYAMNWVRQAPGKGLEWVARIRSKYNNY |
| | VH | | ATYYADSVKGRFTISRDDSKNTAYLQMNNLKTEDTAVYYCVRHGNFGNSYVSWWAYWG |
| | | | QGTLVTVSS |
| 887 | F12Q_LC_L83S- | artificial | QTVVTQEPSLTVSPGGTVTLTCGSSTGAVTSGNYPNWVQQKPGQAPRGLIGGTKFLAP |
| | VL | | GTPARFSGSLSGGKAALTLSGVQPEDEAEYYCVLWYSNRWVFGGGTKLTVL |
| 888 | F12Q_LC_Q46E- | artificial | EVQLVESGGGLVQPGGSLKLSCAASGFTENSYAMNWVRQAPGKGLEWVARIRSKYNNY |
| | VH | | ATYYADSVKGRFTISRDDSKNTAYLQMNNLKTEDTAVYYCVRHGNFGNSYVSWWAYWG |
| | | | QGTLVTVSS |
| 889 | F12Q_LC_Q46E- | artificial | QTVVTQEPSLTVSPGGTVTLTCGSSTGAVTSGNYPNWVQEKPGQAPRGLIGGTKFLAP |
| | VL | | GTPARFSGSLLGGKAALTLSGVQPEDEAEYYCVLWYSNRWVFGGGTKLTVL |
| 890 | F12Q_LC_Q46K- | artificial | EVQLVESGGGLVQPGGSLKLSCAASGFTFNSYAMNWVRQAPGKGLEWVARIRSKYNNY |
| | VH | | ATYYADSVKGRFTISRDDSKNTAYLQMNNLKTEDTAVYYCVRHGNFGNSYVSWWAYWG |
| | | | QGTLVTVSS |
| 891 | F12Q_LC_Q46K- | artificial | QTVVTQEPSLTVSPGGTVTLTCGSSTGAVTSGNYPNWVQKKPGQAPRGLIGGTKFLAP |
| | VL | | GTPARFSGSLLGGKAALTLSGVQPEDEAEYYCVLWYSNRWVFGGGTKLTVL |
| 892 | F12Q_LC_V107A- | artificial | SYAMN |
| | HCDR1 | | |
| 893 | F12Q_LC_V107A- | artificial | RIRSKYNNYATYYADSVKG |
| | HCDR2 | | |
| 894 | F12Q_LC_V107A- | artificial | HGNFGNSYVSWWAY |
| | HCDR3 | | |
| 895 | F12Q_LC_V107A- | artificial | GSSTGAVTSGNYPN |
| | LCDR1 | | |
| 896 | F12Q_LC_V107A- | artificial | GTKFLAP |
| | LCDR2 | | |
| 897 | F12Q_LC_V107A- | artificial | ALWYSNRWV |
| | LCDR3 | | |
| 898 | F12Q_LC_V107A- | artificial | EVQLVESGGGLVQPGGSLKLSCAASGFTFNSYAMNWVRQAPGKGLEWVARIRSKYNNY |
| | VH | | ATYYADSVKGRFTISRDDSKNTAYLQMNNLKTEDTAVYYCVRHGNFGNSYVSWWAYWG |
| | | | QGTLVTVSS |
| 899 | F12Q_LC_V107A- | artificial | QTVVTQEPSLTVSPGGTVTLTCGSSTGAVTSGNYPNWVQQKPGQAPRGLIGGTKFLAP |
| | VL | | GTPARFSGSLLGGKAALTLSGVQPEDEAEYYCALWYSNRWVFGGGTKLTVL |
| 900 | F12Q_LC_V107L- | artificial | SYAMN |
| | HCDR1 | | |
| 901 | F12Q_LC_V107L- | artificial | RIRSKYNNYATYYADSVKG |
| | HCDR2 | | |
| 902 | F12Q_LC_V107L- | artificial | HGNFGNSYVSWWAY |
| | HCDR3 | | |
| 903 | F12Q_LC_V107L- | artificial | GSSTGAVTSGNYPN |
| | LCDR1 | | |
| 904 | F12Q_LC_V107L- | artificial | GTKFLAP |
| | LCDR2 | | |
| 905 | F12Q_LC_V107L- | artificial | LLWYSNRWV |
| | LCDR3 | | |
| 906 | F12Q_LC_V107L- | artificial | EVQLVESGGGLVQPGGSLKLSCAASGFTFNSYAMNWVRQAPGKGLEWVARIRSKYNNY |
| | VH | | ATYYADSVKGRFTISRDDSKNTAYLQMNNLKTEDTAVYYCVRHGNFGNSYVSWWAYWG |
| | | | QGTLVTVSS |
| 907 | F12Q_LC_V107L- | artificial | QTVVTQEPSLTVSPGGTVTLTCGSSTGAVTSGNYPNWVQQKPGQAPRGLIGGTKFLAP |
| | VL | | GTPARFSGSLLGGKAALTLSGVQPEDEAEYYCLLWYSNRWVFGGGTKLTVL |
| 908 | F12Q_LC_V44I- | artificial | EVQLVESGGGLVQPGGSLKLSCAASGFTENSYAMNWVRQAPGKGLEWVARIRSKYNNY |
| | VH | | ATYYADSVKGRFTISRDDSKNTAYLQMNNLKTEDTAVYYCVRHGNFGNSYVSWWAYWG |
| | | | QGTLVTVSS |
| 909 | F12Q_LC_V44I- | artificial | QTVVTQEPSLTVSPGGTVTLTCGSSTGAVTSGNYPNWIQQKPGQAPRGLIGGTKFLAP |
| | VL | | GTPARFSGSLLGGKAALTLSGVQPEDEAEYYCVLWYSNRWVFGGGTKLTVL |
| 910 | F12Q_LC_V44L- | artificial | EVQLVESGGGLVQPGGSLKLSCAASGFTFNSYAMNWVRQAPGKGLEWVARIRSKYNNY |
| | VH | | ATYYADSVKGRFTISRDDSKNTAYLQMNNLKTEDTAVYYCVRHGNFGNSYVSWWAYWG |
| | | | QGTLVTVSS |
| 911 | F12Q_LC_V44L- | artificial | QTVVTQEPSLTVSPGGTVTLTCGSSTGAVTSGNYPNWLQQKPGQAPRGLIGGTKFLAP |
| | VL | | GTPARFSGSLLGGKAALTLSGVQPEDEAEYYCVLWYSNRWVFGGGTKLTVL |
| 912 | F12Q_LC_V44M- | artificial | EVQLVESGGGLVQPGGSLKLSCAASGFTENSYAMNWVRQAPGKGLEWVARIRSKYNNY |
| | VH | | ATYYADSVKGRFTISRDDSKNTAYLQMNNLKTEDTAVYYCVRHGNFGNSYVSWWAYWG |
| | | | QGTLVTVSS |
| 913 | F12Q_LC_V44M- | artificial | QTVVTQEPSLTVSPGGTVTLTCGSSTGAVTSGNYPNWMQQKPGQAPRGLIGGTKFLAP |
| | VL | | GTPARFSGSLLGGKAALTLSGVQPEDEAEYYCVLWYSNRWVFGGGTKLTVL |
| 914 | F12Q_LC_W109Y- | artificial | SYAMN |
| | HCDR1 | | |
| 915 | F12Q_LC_W109Y- | artificial | RIRSKYNNYATYYADSVKG |
| | HCDR2 | | |
| 916 | F12Q_LC_W109Y- | artificial | HGNFGNSYVSWWAY |
| | HCDR3 | | |
| 917 | F12Q_LC_W109Y- | artificial | GSSTGAVTSGNYPN |
| | LCDR1 | | |
| 918 | F12Q_LC_W109Y- | artificial | GTKFLAP |
| | LCDR2 | | |
| 919 | F12Q_LC_W109Y- | artificial | VLYYSNRWV |
| | LCDR3 | | |
| 920 | F12Q_LC_W109Y- | artificial | EVQLVESGGGLVQPGGSLKLSCAASGFTFNSYAMNWVRQAPGKGLEWVARIRSKYNNY |
| | VH | | ATYYADSVKGRFTISRDDSKNTAYLQMNNLKTEDTAVYYCVRHGNFGNSYVSWWAYWG |
| | | | QGTLVTVSS |
| 921 | F12Q_LC_W109Y- | artificial | QTVVTQEPSLTVSPGGTVTLTCGSSTGAVTSGNYPNWVQQKPGQAPRGLIGGTKFLAP |
| | VL | | GTPARFSGSLLGGKAALTLSGVQPEDEAEYYCVLYYSNRWVFGGGTKLTVL |
| 922 | F6A-HCDR1 | artificial | IYAMN |
| 923 | F6A-HCDR2 | artificial | RIRSKYNNYATYYADSVKS |
| 924 | F6A-HCDR3 | artificial | HGNFGNSYVSFFAY |
| 925 | F6A-LCDR1 | artificial | GSSTGAVTSGYYPN |
| 926 | F6A-LCDR2 | artificial | GTKFLAP |
| 927 | F6A-LCDR3 | artificial | ALWYSNRWV |
| 928 | F6A-VH | artificial | EVQLVESGGGLVQPGGSLKLSCAASGFTFNIYAMNWVRQAPGKGLEWVARIRSKYNNY |
| | | | ATYYADSVKSRFTISRDDSKNTAYLQMNNLKTEDTAVYYCVRHGNFGNSYVSFFAYWG |
| | | | QGTLVTVSS |
| 929 | F6A-VL | artificial | QTVVTQEPSLTVSPGGTVTLTCGSSTGAVTSGYYPNWVQQKPGQAPRGLIGGTKFLAP |
| | | | GTPARFSGSLLGGKAALTLSGVQPEDEAEYYCALWYSNRWVFGGGTKLTVL |
| 930 | F6A_HC_A89I | artificial | EVQLVESGGGLVQPGGSLKLSCAASGFTFNIYAMNWVRQAPGKGLEWVARIRSKYNNY |
| | -VH | | ATYYADSVKSRFTISRDDSKNTIYLQMNNLKTEDTAVYYCVRHGNFGNSYVSFFAYWG |
| | | | QGTLVTVSS |
| 931 | F6A_HC_A89I | artificial | QTVVTQEPSLTVSPGGTVTLTCGSSTGAVTSGYYPNWVQQKPGQAPRGLIGGTKFLAP |
| | -VL | | GTPARFSGSLLGGKAALTLSGVQPEDEAEYYCALWYSNRWVFGGGTKLTVL |
| 932 | F6A_HC_A89L | artificial | EVQLVESGGGLVQPGGSLKLSCAASGFTFNIYAMNWVRQAPGKGLEWVARIRSKYNNY |
| | -VH | | ATYYADSVKSRFTISRDDSKNTLYLQMNNLKTEDTAVYYCVRHGNFGNSYVSFFAYWG |
| | | | QGTLVTVSS |
| 933 | F6A_HC_A89L | artificial | QTVVTQEPSLTVSPGGTVTLTCGSSTGAVTSGYYPNWVQQKPGQAPRGLIGGTKFLAP |
| | -VL | | GTPARFSGSLLGGKAALTLSGVQPEDEAEYYCALWYSNRWVFGGGTKLTVL |
| 934 | F6A_HC_A89T | artificial | EVQLVESGGGLVQPGGSLKLSCAASGFTFNIYAMNWVRQAPGKGLEWVARIRSKYNNY |
| | -VH | | ATYYADSVKSRFTISRDDSKNTTYLQMNNLKTEDTAVYYCVRHGNFGNSYVSFFAYWG |
| | | | QGTLVTVSS |
| 935 | F6A_HC_A89T | artificial | QTVVTQEPSLTVSPGGTVTLTCGSSTGAVTSGYYPNWVQQKPGQAPRGLIGGTKFLAP |
| | -VL | | GTPARFSGSLLGGKAALTLSGVQPEDEAEYYCALWYSNRWVFGGGTKLTVL |
| 936 | F6A_HC_A89V | artificial | EVQLVESGGGLVQPGGSLKLSCAASGFTFNIYAMNWVRQAPGKGLEWVARIRSKYNNY |
| | -VH | | ATYYADSVKSRFTISRDDSKNTVYLQMNNLKTEDTAVYYCVRHGNFGNSYVSFFAYWG |
| | | | QGTLVTVSS |
| 937 | F6A_HC_A89V | artificial | QTVVTQEPSLTVSPGGTVTLTCGSSTGAVTSGYYPNWVQQKPGQAPRGLIGGTKFLAP |
| | -VL | | GTPARFSGSLLGGKAALTLSGVQPEDEAEYYCALWYSNRWVFGGGTKLTVL |
| 938 | F6A_HC_L52M | artificial | EVQLVESGGGLVQPGGSLKLSCAASGFTFNIYAMNWVRQAPGKGMEWVARIRSKYNNY |
| | -VH | | ATYYADSVKSRFTISRDDSKNTAYLQMNNLKTEDTAVYYCVRHGNFGNSYVSFFAYWG |
| | | | QGTLVTVSS |
| 939 | F6A_HC_L52M | artificial | QTVVTQEPSLTVSPGGTVTLTCGSSTGAVTSGYYPNWVQQKPGQAPRGLIGGTKFLAP |
| | -VL | | GTPARFSGSLLGGKAALTLSGVQPEDEAEYYCALWYSNRWVFGGGTKLTVL |
| 940 | F6A_HC_L52M_ | artificial | EVQLVESGGGLVQPGGSLKLSCAASGFTFNIYAMNWVRQAPGKGMEWVARIRSKYNNY |
| | LC_L21I_ | | ATYYADSVKSRFTISRDDSKNTAYLQMNNLKTEDTAVYYCVRHGNFGNSYVSFFAYWG |
| | Q46K_G141S- | | QGTLVTVSS |
| | VH | | |
| 941 | F6A_HC_L52M_ | artificial | QTVVTQEPSLTVSPGGTVTITCGSSTGAVTSGYYPNWVQKKPGQAPRGLIGGTKFLAP |
| | LC_L21I_ | | GTPARFSGSLLGGKAALTLSGVQPEDEAEYYCALWYSNRWVFGSGTKLTVL |
| | Q46K_G141S- | | |
| | VL | | |
| 942 | F6A_HC_L52M_ | artificial | EVQLVESGGGLVQPGGSLKLSCAASGFTFNIYAMNWVRQAPGKGMEWVARIRSKYNNY |
| | LC_Q46K- | | ATYYADSVKSRFTISRDDSKNTAYLQMNNLKTEDTAVYYCVRHGNFGNSYVSFFAYWG |
| | VH | | QGTLVTVSS |
| 943 | F6A_HC_L52M_ | artificial | QTVVTQEPSLTVSPGGTVTLTCGSSTGAVTSGYYPNWVQKKPGQAPRGLIGGTKFLAP |
| | LC_Q46K- | | GTPARFSGSLLGGKAALTLSGVQPEDEAEYYCALWYSNRWVFGGGTKLTVL |
| | VL | | |
| 944 | F6A_HC_L52M_ | artificial | EVQLVESGGGLVQPGGSLKLSCAASGFTFNIYAMNWVRQAPGKGMEWVARIRSKYNNY |
| | LC_Q46K_ | | ATYYADSVKSRFTISRDDSKNTAYLQMNNLKTEDTAVYYCVRHGNFGNSYVSFFAYWG |
| | G141S-VH | | QGTLVTVSS |
| 945 | F6A_HC_L52M_ | artificial | QTVVTQEPSLTVSPGGTVTLTCGSSTGAVTSGYYPNWVQKKPGQAPRGLIGGTKFLAP |
| | LC_Q46K_ | | GTPARFSGSLLGGKAALTLSGVQPEDEAEYYCALWYSNRWVFGSGTKLTVL |
| | G141S-VL | | |
| 946 | F6A_HC_L52V | artificial | EVQLVESGGGLVQPGGSLKLSCAASGFTFNIYAMNWVRQAPGKGVEWVARIRSKYNNY |
| | -VH | | ATYYADSVKSRFTISRDDSKNTAYLQMNNLKTEDTAVYYCVRHGNFGNSYVSFFAYWG |
| | | | QGTLVTVSS |
| 947 | F6A_HC_L52V | artificial | QTVVTQEPSLTVSPGGTVTLTCGSSTGAVTSGYYPNWVQQKPGQAPRGLIGGTKFLAP |
| | -VL | | GTPARFSGSLLGGKAALTLSGVQPEDEAEYYCALWYSNRWVFGGGTKLTVL |
| 948 | F6A_HC_L52V | artificial | EVQLVESGGGLVQPGGSLKLSCAASGFTFNIYAMNWVRQAPGKGVEWVARIRSKYNNY |
| | LC_V44F- | | ATYYADSVKSRFTISRDDSKNTAYLQMNNLKTEDTAVYYCVRHGNFGNSYVSFFAYWG |
| | VH | | QGTLVTVSS |
| 949 | F6A_HC_L52V_ | artificial | QTVVTQEPSLTVSPGGTVTLTCGSSTGAVTSGYYPNWFQQKPGQAPRGLIGGTKFLAP |
| | LC_V44F- | | GTPARFSGSLLGGKAALTLSGVQPEDEAEYYCALWYSNRWVFGGGTKLTVL |
| | VL | | |
| 950 | F6A_HC_L52V_ | artificial | EVQLVESGGGLVQPGGSLKLSCAASGFTFNIYAMNWVRQAPGKGVEWVARIRSKYNNY |
| | LC_V44Y- | | ATYYADSVKSRFTISRDDSKNTAYLQMNNLKTEDTAVYYCVRHGNFGNSYVSFFAYWG |
| | VH | | QGTLVTVSS |
| 951 | F6A_HC_L52V_ | artificial | QTVVTQEPSLTVSPGGTVTLTCGSSTGAVTSGYYPNWYQQKPGQAPRGLIGGTKFLAP |
| | LC_V44Y- | | GTPARFSGSLLGGKAALTLSGVQPEDEAEYYCALWYSNRWVFGGGTKLTVL |
| | VL | | |
| 952 | F6A_HC_M41F_ | artificial | IYAFN |
| | A89V- | | |
| | HCDR1 | | |
| 953 | F6A_HC_M41F_ | artificial | RIRSKYNNYATYYADSVKS |
| | A89V- | | |
| | HCDR2 | | |
| 954 | F6A_HC_M41F_ | artificial | HGNFGNSYVSFFAY |
| | A89V- | | |
| | HCDR3 | | |
| 955 | F6A_HC_M41F_ | artificial | GSSTGAVTSGYYPN |
| | A89V- | | |
| | LCDR1 | | |
| 956 | F6A_HC_M41F_ | artificial | GTKFLAP |
| | A89V-LCDR2 | | |
| 957 | F6A_HC_M41F_ | artificial | ALWYSNRWV |
| | A89V-LCDR3 | | |
| 958 | F6A_HC_M41F_ | artificial | EVQLVESGGGLVQPGGSLKLSCAASGFTFNIYAFNWVRQAPGKGLEWVARIRSKYNNY |
| | A89V-VH | | ATYYADSVKSRFTISRDDSKNTVYLQMNNLKTEDTAVYYCVRHGNFGNSYVSFFAYWG |
| | | | QGTLVTVSS |
| 959 | F6A_HC_M41F_ | artificial | QTVVTQEPSLTVSPGGTVTLTCGSSTGAVTSGYYPNWVQQKPGQAPRGLIGGTKFLAP |
| | A89V-VL | | GTPARFSGSLLGGKAALTLSGVQPEDEAEYYCALWYSNRWVFGGGTKLTVL |
| 960 | F6A_HC_M41I | artificial | IYAIN |
| | -HCDR1 | | |
| 961 | F6A_HC_M41I | artificial | RIRSKYNNYATYYADSVKS |
| | -HCDR2 | | |
| 962 | F6A_HC_M41I | artificial | HGNFGNSYVSFFAY |
| | -HCDR3 | | |
| 963 | F6A_HC_M41I | artificial | GSSTGAVTSGYYPN |
| | -LCDR1 | | |
| 964 | F6A_HC_M41I | artificial | GTKFLAP |
| | -LCDR2 | | |
| 965 | F6A_HC_M41I | artificial | ALWYSNRWV |
| | -LCDR3 | | |
| 966 | F6A_HC_M41I | artificial | EVQLVESGGGLVQPGGSLKLSCAASGFTFNIYAINWVRQAPGKGLEWVARIRSKYNNY |
| | -VH | | ATYYADSVKSRFTISRDDSKNTAYLQMNNLKTEDTAVYYCVRHGNFGNSYVSFFAYWG |
| | | | QGTLVTVSS |
| 967 | F6A_HC_M41I | artificial | QTVVTQEPSLTVSPGGTVTLTCGSSTGAVTSGYYPNWVQQKPGQAPRGLIGGTKFLAP |
| | -VL | | GTPARFSGSLLGGKAALTLSGVQPEDEAEYYCALWYSNRWVFGGGTKLTVL |
| 968 | F6A_HC_M41I_ | artificial | EVQLVESGGGLVQPGGSLKLSCAASGFTFNIYAINWVRQAPGKGLEWVARIRSKYNNY |
| | A89L-VH | | ATYYADSVKSRFTISRDDSKNTLYLQMNNLKTEDTAVYYCVRHGNFGNSYVSFFAYWG |
| | | | QGTLVTVSS |
| 969 | F6A_HC_M41I_ | artificial | QTVVTQEPSLTVSPGGTVTLTCGSSTGAVTSGYYPNWVQQKPGQAPRGLIGGTKFLAP |
| | A89L-VL | | GTPARFSGSLLGGKAALTLSGVQPEDEAEYYCALWYSNRWVFGGGTKLTVL |
| 970 | F6A_HC_M41I_ | artificial | EVQLVESGGGLVQPGGSLKLSCAASGFTFNIYAINWVRQAPGKGLEWVARIRSKYNNY |
| | A89V-VH | | ATYYADSVKSRFTISRDDSKNTVYLQMNNLKTEDTAVYYCVRHGNFGNSYVSFFAYWG |
| | | | QGTLVTVSS |
| 971 | F6A_HC_M41I_ | artificial | QTVVTQEPSLTVSPGGTVTLTCGSSTGAVTSGYYPNWVQQKPGQAPRGLIGGTKFLAP |
| | A89V-VL | | GTPARFSGSLLGGKAALTLSGVQPEDEAEYYCALWYSNRWVFGGGTKLTVL |
| 972 | F6A_HC_M41I_ | artificial | EVQLVESGGGLVQPGGSLKLSCAASGFTFNIYAINWVRQAPGKGLEWVARIRSKYNNY |
| | A89V_LC_ | | ATYYADSVKSRFTISRDDSKNTVYLQMNNLKTEDTAVYYCVRHGNFGNSYVSFFAYWG |
| | L21I_Q46K_ | | QGTLVTVSS |
| | G141S-VH | | |
| 973 | F6A_HC_M41I_ | artificial | QTVVTQEPSLTVSPGGTVTITCGSSTGAVTSGYYPNWVQKKPGQAPRGLIGGTKFLAP |
| | A89V_LC_ | | GTPARFSGSLLGGKAALTLSGVQPEDEAEYYCALWYSNRWVFGSGTKLTVL |
| | L21I_Q46K_ | | |
| | G141S-VL | | |
| 974 | F6A_HC_M41I_ | artificial | EVQLVESGGGLVQPGGSLKLSCAASGFTFNIYAINWVRQAPGKGLEWVARIRSKYNNY |
| | A89V_LC_ | | ATYYADSVKSRFTISRDDSKNTVYLQMNNLKTEDTAVYYCVRHGNFGNSYVSFFAYWG |
| | Q46K-VH | | QGTLVTVSS |
| 975 | F6A_HC_M41I_ | artificial | QTVVTQEPSLTVSPGGTVTLTCGSSTGAVTSGYYPNWVQKKPGQAPRGLIGGTKFLAP |
| | A89V_LC_ | | GTPARFSGSLLGGKAALTLSGVQPEDEAEYYCALWYSNRWVFGGGTKLTVL |
| | Q46K-VL | | |
| 976 | F6A_HC_M41I_ | artificial | EVQLVESGGGLVQPGGSLKLSCAASGFTFNIYAINWVRQAPGKGVEWVARIRSKYNNY |
| | L52V_A89V_ | | ATYYADSVKSRFTISRDDSKNTVYLQMNNLKTEDTAVYYCVRHGNFGNSYVSFFAYWG |
| | LC_L21I_ | | QGTLVTVSS |
| | Q46K_G141S_ | | |
| | V44F-VH | | |
| 977 | F6A_HC_M41I_ | artificial | QTVVTQEPSLTVSPGGTVTITCGSSTGAVTSGYYPNWFQKKPGQAPRGLIGGTKFLAP |
| | L52V_A89V_ | | GTPARFSGSLLGGKAALTLSGVQPEDEAEYYCALWYSNRWVFGSGTKLTVL |
| | LC_L21I_ | | |
| | Q46K_G141S_ | | |
| | V44F-VL | | |
| 978 | F6A_HC_M41I_ | artificial | EVQLVESGGGLVQPGGSLKLSCAASGFTFNIYAINWVRQAPGKGLEWVARIRSKYNNY |
| | LC_L21I_ | | ATYYADSVKSRFTISRDDSKNTAYLQMNNLKTEDTAVYYCVRHGNFGNSYVSFFAYWG |
| | Q46K_G141S- | | QGTLVTVSS |
| | VH | | |
| 979 | F6A_HC_M41I_ | artificial | QTVVTQEPSLTVSPGGTVTITCGSSTGAVTSGYYPNWVQKKPGQAPRGLIGGTKFLAP |
| | LC_L21I_ | | GTPARFSGSLLGGKAALTLSGVQPEDEAEYYCALWYSNRWVFGSGTKLTVL |
| | Q46K_G141S- | | |
| | VL | | |
| 980 | F6A_HC_M41I_ | artificial | EVQLVESGGGLVQPGGSLKLSCAASGFTFNIYAINWVREAPGKGLEWVARIRSKYNNY |
| | Q46E_A89V_ | | ATYYADSVKSRFTISRDDSKNTVYLQMNNLKTEDTAVYYCVRHGNFGNSYVSFFAYWG |
| | LC_L21I_ | | QGTLVTVSS |
| | Q46K_G141S- | | |
| | VH | | |
| 981 | F6A_HC_M41I_ | artificial | QTVVTQEPSLTVSPGGTVTITCGSSTGAVTSGYYPNWVQKKPGQAPRGLIGGTKFLAP |
| | Q46E_A89V_ | | GTPARFSGSLLGGKAALTLSGVQPEDEAEYYCALWYSNRWVFGSGTKLTVL |
| | LC_L21I_ | | |
| | Q46K_G141S- | | |
| | VL | | |
| 982 | F6A_HC_M41V | artificial | IYAVN |
| | -HCDR1 | | |
| 983 | F6A_HC_M41V | artificial | RIRSKYNNYATYYADSVKS |
| | -HCDR2 | | |
| 984 | F6A_HC_M41V | artificial | HGNFGNSYVSFFAY |
| | -HCDR3 | | |
| 985 | F6A_HC_M41V | artificial | GSSTGAVTSGYYPN |
| | -LCDR1 | | |
| 986 | F6A_HC_M41V | artificial | GTKFLAP |
| | -LCDR2 | | |
| 987 | F6A_HC_M41V | artificial | ALWYSNRWV |
| | -LCDR3 | | |
| 988 | F6A_HC_M41V | artificial | EVQLVESGGGLVQPGGSLKLSCAASGFTFNIYAVNWVRQAPGKGLEWVARIRSKYNNY |
| | -VH | | ATYYADSVKSRFTISRDDSKNTAYLQMNNLKTEDTAVYYCVRHGNFGNSYVSFFAYWG |
| | | | QGTLVTVSS |
| 989 | F6A_HC_M41V | artificial | QTVVTQEPSLTVSPGGTVTLTCGSSTGAVTSGYYPNWVQQKPGQAPRGLIGGTKFLAP |
| | -VL | | GTPARFSGSLLGGKAALTLSGVQPEDEAEYYCALWYSNRWVFGGGTKLTVL |
| 990 | F6A_HC_Q46D | artificial | EVQLVESGGGLVQPGGSLKLSCAASGFTFNIYAMNWVRDAPGKGLEWVARIRSKYNNY |
| | -VH | | ATYYADSVKSRFTISRDDSKNTAYLQMNNLKTEDTAVYYCVRHGNFGNSYVSFFAYWG |
| | | | QGTLVTVSS |
| 991 | F6A_HC_Q46D | artificial | QTVVTQEPSLTVSPGGTVTLTCGSSTGAVTSGYYPNWVQQKPGQAPRGLIGGTKFLAP |
| | -VL | | GTPARFSGSLLGGKAALTLSGVQPEDEAEYYCALWYSNRWVFGGGTKLTVL |
| 992 | F6A_HC_Q46D_ | artificial | EVQLVESGGGLVQPGGSLKLSCAASGFTFNIYAMNWVRDAPGKGLEWVARIRSKYNNY |
| | LC_L21I_ | | ATYYADSVKSRFTISRDDSKNTAYLQMNNLKTEDTAVYYCVRHGNFGNSYVSFFAYWG |
| | Q46K_G141S- | | QGTLVTVSS |
| | VH | | |
| 993 | F6A_HC_Q46D_ | artificial | QTVVTQEPSLTVSPGGTVTITCGSSTGAVTSGYYPNWVQKKPGQAPRGLIGGTKFLAP |
| | LC_L21I_ | | GTPARFSGSLLGGKAALTLSGVQPEDEAEYYCALWYSNRWVFGSGTKLTVL |
| | Q46K_G141S- | | |
| | VL | | |
| 994 | F6A_HC_Q46D_ | artificial | EVQLVESGGGLVQPGGSLKLSCAASGFTFNIYAMNWVRDAPGKGLEWVARIRSKYNNY |
| | LC_L21I_ | | ATYYADSVKSRFTISRDDSKNTAYLQMNNLKTEDTAVYYCVRHGNFGNSYVSFFAYWG |
| | Q46R_G141S- | | QGTLVTVSS |
| | VH | | |
| 995 | F6A_HC_Q46D_ | artificial | QTVVTQEPSLTVSPGGTVTITCGSSTGAVTSGYYPNWVQRKPGQAPRGLIGGTKFLAP |
| | LC_L21I_ | | GTPARFSGSLLGGKAALTLSGVQPEDEAEYYCALWYSNRWVFGSGTKLTVL |
| | Q46R_G141S- | | |
| | VL | | |
| 996 | F6A_HC_Q46D_ | artificial | EVQLVESGGGLVQPGGSLKLSCAASGFTFNIYAMNWVRDAPGKGLEWVARIRSKYNNY |
| | LC_Q46K- | | ATYYADSVKSRFTISRDDSKNTAYLQMNNLKTEDTAVYYCVRHGNFGNSYVSFFAYWG |
| | VH | | QGTLVTVSS |
| 997 | F6A_HC_Q46D_ | artificial | QTVVTQEPSLTVSPGGTVTLTCGSSTGAVTSGYYPNWVQKKPGQAPRGLIGGTKFLAP |
| | LC_Q46K- | | GTPARFSGSLLGGKAALTLSGVQPEDEAEYYCALWYSNRWVFGGGTKLTVL |
| | VL | | |
| 998 | F6A_HC_Q46D_ | artificial | EVQLVESGGGLVQPGGSLKLSCAASGFTFNIYAMNWVRDAPGKGLEWVARIRSKYNNY |
| | LC_Q46K_ | | ATYYADSVKSRFTISRDDSKNTAYLQMNNLKTEDTAVYYCVRHGNFGNSYVSFFAYWG |
| | G141S-VH | | QGTLVTVSS |
| 999 | F6A_HC_Q46D_ | artificial | QTVVTQEPSLTVSPGGTVTLTCGSSTGAVTSGYYPNWVQKKPGQAPRGLIGGTKFLAP |
| | LC_Q46K_ | | GTPARFSGSLLGGKAALTLSGVQPEDEAEYYCALWYSNRWVFGSGTKLTVL |
| | G141S-VL | | |
| 1000 | F6A_HC_Q46D_ | artificial | EVQLVESGGGLVQPGGSLKLSCAASGFTFNIYAMNWVRDAPGKGLEWVARIRSKYNNY |
| | LC_Q46R- | | ATYYADSVKSRFTISRDDSKNTAYLQMNNLKTEDTAVYYCVRHGNFGNSYVSFFAYWG |
| | VH | | QGTLVTVSS |
| 1001 | F6A_HC_Q46D_ | artificial | QTVVTQEPSLTVSPGGTVTLTCGSSTGAVTSGYYPNWVQRKPGQAPRGLIGGTKFLAP |
| | LC_Q46R- | | GTPARFSGSLLGGKAALTLSGVQPEDEAEYYCALWYSNRWVFGGGTKLTVL |
| | VL | | |
| 1002 | F6A_HC_Q46D_ | artificial | EVQLVESGGGLVQPGGSLKLSCAASGFTFNIYAMNWVRDAPGKGLEWVARIRSKYNNY |
| | LC_Q46R_ | | ATYYADSVKSRFTISRDDSKNTAYLQMNNLKTEDTAVYYCVRHGNFGNSYVSFFAYWG |
| | G141S-VH | | QGTLVTVSS |
| 1003 | F6A_HC_Q46D_ | artificial | QTVVTQEPSLTVSPGGTVTLTCGSSTGAVTSGYYPNWVQRKPGQAPRGLIGGTKFLAP |
| | LC_Q46R_ | | GTPARFSGSLLGGKAALTLSGVQPEDEAEYYCALWYSNRWVFGSGTKLTVL |
| | G141S-VL | | |
| 1004 | F6A_HC_Q46E | artificial | EVQLVESGGGLVQPGGSLKLSCAASGFTFNIYAMNWVREAPGKGLEWVARIRSKYNNY |
| | -VH | | ATYYADSVKSRFTISRDDSKNTAYLQMNNLKTEDTAVYYCVRHGNFGNSYVSFFAYWG |
| | | | QGTLVTVSS |
| 1005 | F6A_HC_Q46E | artificial | QTVVTQEPSLTVSPGGTVTLTCGSSTGAVTSGYYPNWVQQKPGQAPRGLIGGTKFLAP |
| | -VL | | GTPARFSGSLLGGKAALTLSGVQPEDEAEYYCALWYSNRWVFGGGTKLTVL |
| 1006 | F6A_HC_Q46E_ | artificial | EVQLVESGGGLVQPGGSLKLSCAASGFTFNIYAMNWVREAPGKGLEWVARIRSKYNNY |
| | LC_L21I_ | | ATYYADSVKSRFTISRDDSKNTAYLQMNNLKTEDTAVYYCVRHGNFGNSYVSFFAYWG |
| | Q46K_G141S- | | QGTLVTVSS |
| | VH | | |
| 1007 | F6A_HC_Q46E_ | artificial | QTVVTQEPSLTVSPGGTVTITCGSSTGAVTSGYYPNWVQKKPGQAPRGLIGGTKFLAP |
| | LC_L21I_ | | GTPARFSGSLLGGKAALTLSGVQPEDEAEYYCALWYSNRWVFGSGTKLTVL |
| | Q46K_G141S- | | |
| | VL | | |
| 1008 | F6A_HC_Q46E_ | artificial | EVQLVESGGGLVQPGGSLKLSCAASGFTFNIYAMNWVREAPGKGLEWVARIRSKYNNY |
| | LC_L21I_ | | ATYYADSVKSRFTISRDDSKNTAYLQMNNLKTEDTAVYYCVRHGNFGNSYVSFFAYWG |
| | Q46R_G141S- | | QGTLVTVSS |
| | VH | | |
| 1009 | F6A_HC_Q46E_ | artificial | QTVVTQEPSLTVSPGGTVTITCGSSTGAVTSGYYPNWVQRKPGQAPRGLIGGTKFLAP |
| | LC_L21I_ | | GTPARFSGSLLGGKAALTLSGVQPEDEAEYYCALWYSNRWVFGSGTKLTVL |
| | Q46R_G141S- | | |
| | VL | | |
| 1010 | F6A_HC_Q46E_ | artificial | EVQLVESGGGLVQPGGSLKLSCAASGFTFNIYAMNWVREAPGKGLEWVARIRSKYNNY |
| | LC_Q46K- | | ATYYADSVKSRFTISRDDSKNTAYLQMNNLKTEDTAVYYCVRHGNFGNSYVSFFAYWG |
| | VH | | QGTLVTVSS |
| 1011 | F6A_HC_Q46E_ | artificial | QTVVTQEPSLTVSPGGTVTLTCGSSTGAVTSGYYPNWVQKKPGQAPRGLIGGTKFLAP |
| | LC_Q46K- | | GTPARFSGSLLGGKAALTLSGVQPEDEAEYYCALWYSNRWVFGGGTKLTVL |
| | VL | | |
| 1012 | F6A_HC_Q46E_ | artificial | EVQLVESGGGLVQPGGSLKLSCAASGFTFNIYAMNWVREAPGKGLEWVARIRSKYNNY |
| | LC_Q46K_ | | ATYYADSVKSRFTISRDDSKNTAYLQMNNLKTEDTAVYYCVRHGNFGNSYVSFFAYWG |
| | G141S-VH | | QGTLVTVSS |
| 1013 | F6A_HC_Q46E_ | artificial | QTVVTQEPSLTVSPGGTVTLTCGSSTGAVTSGYYPNWVQKKPGQAPRGLIGGTKFLAP |
| | LC_Q46K_ | | GTPARFSGSLLGGKAALTLSGVQPEDEAEYYCALWYSNRWVFGSGTKLTVL |
| | G141S-VL | | |
| 1014 | F6A_HC_Q46E_ | artificial | EVQLVESGGGLVQPGGSLKLSCAASGFTFNIYAMNWVREAPGKGLEWVARIRSKYNNY |
| | LC_Q46R- | | ATYYADSVKSRFTISRDDSKNTAYLQMNNLKTEDTAVYYCVRHGNFGNSYVSFFAYWG |
| | VH | | QGTLVTVSS |
| 1015 | F6A_HC_Q46E_ | artificial | QTVVTQEPSLTVSPGGTVTLTCGSSTGAVTSGYYPNWVQRKPGQAPRGLIGGTKFLAP |
| | LC_Q46R- | | GTPARFSGSLLGGKAALTLSGVQPEDEAEYYCALWYSNRWVFGGGTKLTVL |
| | VL | | |
| 1016 | F6A_HC_Q46E_ | artificial | EVQLVESGGGLVQPGGSLKLSCAASGFTFNIYAMNWVREAPGKGLEWVARIRSKYNNY |
| | LC_Q46R_ | | ATYYADSVKSRFTISRDDSKNTAYLQMNNLKTEDTAVYYCVRHGNFGNSYVSFFAYWG |
| | G141S-VH | | QGTLVTVSS |
| 1017 | F6A_HC_Q46E_ | artificial | QTVVTQEPSLTVSPGGTVTLTCGSSTGAVTSGYYPNWVQRKPGQAPRGLIGGTKFLAP |
| | LC_Q46R_ | | GTPARFSGSLLGGKAALTLSGVQPEDEAEYYCALWYSNRWVFGSGTKLTVL |
| | G141S-VL | | |
| 1018 | F6A_HC_Q46K | artificial | EVQLVESGGGLVQPGGSLKLSCAASGFTFNIYAMNWVRKAPGKGLEWVARIRSKYNNY |
| | -VH | | ATYYADSVKSRFTISRDDSKNTAYLQMNNLKTEDTAVYYCVRHGNFGNSYVSFFAYWG |
| | | | QGTLVTVSS |
| 1019 | F6A_HC_Q46K | artificial | QTVVTQEPSLTVSPGGTVTLTCGSSTGAVTSGYYPNWVQQKPGQAPRGLIGGTKFLAP |
| | -VL | | GTPARFSGSLLGGKAALTLSGVQPEDEAEYYCALWYSNRWVFGGGTKLTVL |
| 1020 | F6A_HC_Q46R | artificial | EVQLVESGGGLVQPGGSLKLSCAASGFTFNIYAMNWVRRAPGKGLEWVARIRSKYNNY |
| | -VH | | ATYYADSVKSRFTISRDDSKNTAYLQMNNLKTEDTAVYYCVRHGNFGNSYVSFFAYWG |
| | | | QGTLVTVSS |
| 1021 | F6A_HC_Q46R | artificial | QTVVTQEPSLTVSPGGTVTLTCGSSTGAVTSGYYPNWVQQKPGQAPRGLIGGTKFLAP |
| | -VL | | GTPARFSGSLLGGKAALTLSGVQPEDEAEYYCALWYSNRWVFGGGTKLTVL |
| 1022 | F6A_HC_V107A- | artificial | EVQLVESGGGLVQPGGSLKLSCAASGFTFNIYAMNWVRQAPGKGLEWVARIRSKYNNY |
| | VH | | ATYYADSVKSRFTISRDDSKNTAYLQMNNLKTEDTAVYYCARHGNFGNSYVSFFAYWG |
| | | | QGTLVTVSS |
| 1023 | F6A_HC_V107A- | artificial | QTVVTQEPSLTVSPGGTVTLTCGSSTGAVTSGYYPNWVQQKPGQAPRGLIGGTKFLAP |
| | VL | | GTPARFSGSLLGGKAALTLSGVQPEDEAEYYCALWYSNRWVFGGGTKLTVL |
| 1024 | F6A_HC_V107L- | artificial | EVQLVESGGGLVQPGGSLKLSCAASGFTFNIYAMNWVRQAPGKGLEWVARIRSKYNNY |
| | VH | | ATYYADSVKSRFTISRDDSKNTAYLQMNNLKTEDTAVYYCLRHGNFGNSYVSFFAYWG |
| | | | QGTLVTVSS |
| 1025 | F6A_HC_V107L- | artificial | QTVVTQEPSLTVSPGGTVTLTCGSSTGAVTSGYYPNWVQQKPGQAPRGLIGGTKFLAP |
| | VL | | GTPARFSGSLLGGKAALTLSGVQPEDEAEYYCALWYSNRWVFGGGTKLTVL |
| 1026 | F6A_I2E- | artificial | IYAIN |
| | HCDR1 | | |
| 1027 | F6A_I2E- | artificial | RIRSKYNNYATYYADAVKS |
| | HCDR2 | | |
| 1028 | F6A_I2E- | artificial | AGNFGSSYVSFFAY |
| | HCDR3 | | |
| 1029 | F6A_I2E- | artificial | GSSTGAVTSGYYPN |
| | LCDR1 | | |
| 1030 | F6A_I2E- | artificial | GTKFLAP |
| | LCDR2 | | |
| 1031 | F6A_I2E- | artificial | ALWYSNRWV |
| | LCDR3 | | |
| 1032 | F6A_I2E- | artificial | EVQLVESGGGLVQPGGSLKLSCAASGFTFNIYAINWVRQAPGKGLEWVARIRSKYNNY |
| | VH | | ATYYADAVKSRFTISRDDSKNTVYLQMNNLKTEDTAVYYCARAGNFGSSYVSFFAYWG |
| | | | QGTLVTVSS |
| 1033 | F6A_I2E- | artificial | QTVVTQEPSLTVSPGGTVTITCGSSTGAVTSGYYPNWVQKKPGQAPRGLIGGTKFLAP |
| | VL | | GTPARFSGSLSGGKAALTLSGVQPEDEAEYYCALWYSNRWVFGSGTKLTVL |
| 1034 | F6A_I2K- | artificial | IYAMN |
| | HCDR1 | | |
| 1035 | F6A_I2K- | artificial | RIRSKYNNYATYYAEAVKG |
| | HCDR2 | | |
| 1036 | F6A_I2K- | artificial | NENIGTSYVSFFAY |
| | HCDR3 | | |
| 1037 | F6A_I2K- | artificial | GSSTGAVTSGYYPN |
| | LCDR1 | | |
| 1038 | F6A_I2K- | artificial | GTKFLAP |
| | LCDR2 | | |
| 1039 | F6A_I2K- | artificial | ALWYSNRWV |
| | LCDR3 | | |
| 1040 | F6A_I2K- | artificial | EVQLVESGGGLVQPGGSLKLSCAASGFTFNIYAMNWVRQAPGKGLEWVARIRSKYNNY |
| | VH | | ATYYAEAVKGRFTISRDDSKNTVYLQMNNLKTEDTAVYYCVRNENIGTSYVSFFAYWG |
| | | | QGTLVTVSS |
| 1041 | F6A_I2K- | artificial | QTVVTQEPSLTVSPGGTVTITCGSSTGAVTSGYYPNWVQQKPGQAPRGLIGGTKFLAP |
| | VL | | GTPARFSGSLSGGKAALTLSGVQPEDEAEYYCALWYSNRWVFGSGTKLTVL |
| 1042 | F6A_I2L- | artificial | IYAMN |
| | HCDR1 | | |
| 1043 | F6A_I2L- | artificial | RIRSKYNNYATYYADAVKS |
| | HCDR2 | | |
| 1044 | F6A_I2L- | artificial | AGNFGSSYVSFFAY |
| | HCDR3 | | |
| 1045 | F6A_I2L- | artificial | GSSTGAVTSGYYPN |
| | LCDR1 | | |
| 1046 | F6A_I2L- | artificial | GTKFLAP |
| | LCDR2 | | |
| 1047 | F6A_I2L- | artificial | ALYYSNRWV |
| | LCDR3 | | |
| 1048 | F6A_I2L- | artificial | EVQLVESGGGLVQPGGSLKLSCAASGFTFNIYAMNWVRQAPGKGMEWVARIRSKYNNY |
| | VH | | ATYYADAVKSRFTISRDDSKNTLYLQMNNLKTEDTAVYYCVRAGNFGSSYVSFFAYWG |
| | | | QGTLVTVSS |
| 1049 | F6A_I2L- | artificial | QTVVTQEPSLTVSPGGTVTITCGSSTGAVTSGYYPNWIQKKPGQAPRGLIGGTKFLAP |
| | VL | | GTPARFSGSLEGGKAALTLSGVQPEDEAEYYCALYYSNRWVFGSGTKLTVL |
| 1050 | F6A_I2M- | artificial | IYAMN |
| | HCDR1 | | |
| 1051 | F6A_I2M- | artificial | RIRSKYNNYATYYADAVKS |
| | HCDR2 | | |
| 1052 | F6A_I2M- | artificial | AGNFGTSYVSFFAY |
| | HCDR3 | | |
| 1053 | F6A_I2M- | artificial | GSSTGAVTSGYYPN |
| | LCDR1 | | |
| 1054 | F6A_I2M- | artificial | GTKFLAP |
| | LCDR2 | | |
| 1055 | F6A_I2M- | artificial | ALWYSNRWV |
| | LCDR3 | | |
| 1056 | F6A_I2M- | artificial | EVQLVESGGGLVQPGGSLKLSCAASGFTFNIYAMNWVRQAPGKGMEWVARIRSKYNNY |
| | VH | | ATYYADAVKSRFTISRDDSKNTLYLQMNNLKTEDTAVYYCVRAGNFGTSYVSFFAYWG |
| | | | QGTLVTVSS |
| 1057 | F6A_I2M- | artificial | QTVVTQEPSLTVSPGGTVTITCGSSTGAVTSGYYPNWVQKKPGQAPRGLIGGTKFLAP |
| | VL | | GTPARFSGSLSGGKAALTLSGVQPEDEAEYYCALWYSNRWVFGSGTKLTVL |
| 1058 | F6A_I2M2- | artificial | IYAIN |
| | HCDR1 | | |
| 1059 | F6A_I2M2- | artificial | RIRSKYNNYATYYADAVKS |
| | HCDR2 | | |
| 1060 | F6A_I2M2- | artificial | NANFGTSYVSFFAY |
| | HCDR3 | | |
| 1061 | F6A_I2M2- | artificial | GSSTGAVTSGYYPN |
| | LCDR1 | | |
| 1062 | F6A_I2M2- | artificial | GTKFLAP |
| | LCDR2 | | |
| 1063 | F6A_I2M2- | artificial | ALWYSNRWV |
| | LCDR3 | | |
| 1064 | F6A_I2M2- | artificial | EVQLVESGGGLVQPGGSLKLSCAASGFTFNIYAINWVREAPGKGLEWVARIRSKYNNY |
| | VH | | ATYYADAVKSRFTISRDDSKNTAYLQMNNLKTEDTAVYYCVRNANFGTSYVSFFAYWG |
| | | | QGTLVTVSS |
| 1065 | F6A_I2M2- | artificial | QTVVTQEPSLTVSPGGTVTLTCGSSTGAVTSGYYPNWVQKKPGQAPRGLIGGTKFLAP |
| | VL | | GTPARFSGSLLGGKAALTLSGVQPEDEAEYYCALWYSNRWVFGSGTKLTVL |
| 1066 | F6A_LC_G141S- | artificial | EVQLVESGGGLVQPGGSLKLSCAASGFTFNIYAMNWVRQAPGKGLEWVARIRSKYNNY |
| | VH | | ATYYADSVKSRFTISRDDSKNTAYLQMNNLKTEDTAVYYCVRHGNFGNSYVSFFAYWG |
| | | | QGTLVTVSS |
| 1067 | F6A_LC_G141S- | artificial | QTVVTQEPSLTVSPGGTVTLTCGSSTGAVTSGYYPNWVQQKPGQAPRGLIGGTKFLAP |
| | VL | | GTPARFSGSLLGGKAALTLSGVQPEDEAEYYCALWYSNRWVFGSGTKLTVL |
| 1068 | F6A_LC_G141S_ | artificial | EVQLVESGGGLVQPGGSLKLSCAASGFTFNIYAMNWVRQAPGKGLEWVARIRSKYNNY |
| | L21I-VH | | ATYYADSVKSRFTISRDDSKNTAYLQMNNLKTEDTAVYYCVRHGNFGNSYVSFFAYWG |
| | | | QGTLVTVSS |
| 1069 | F6A_LC_G141S_ | artificial | QTVVTQEPSLTVSPGGTVTITCGSSTGAVTSGYYPNWVQQKPGQAPRGLIGGTKFLAP |
| | L21I-VL | | GTPARFSGSLLGGKAALTLSGVQPEDEAEYYCALWYSNRWVFGSGTKLTVL |
| 1070 | F6A_LC_L83E | artificial | EVQLVESGGGLVQPGGSLKLSCAASGFTFNIYAMNWVRQAPGKGLEWVARIRSKYNNY |
| | -VH | | ATYYADSVKSRFTISRDDSKNTAYLQMNNLKTEDTAVYYCVRHGNFGNSYVSFFAYWG |
| | | | QGTLVTVSS |
| 1071 | F6A_LC_L83E | artificial | QTVVTQEPSLTVSPGGTVTLTCGSSTGAVTSGYYPNWVQQKPGQAPRGLIGGTKFLAP |
| | -VL | | GTPARFSGSLEGGKAALTLSGVQPEDEAEYYCALWYSNRWVFGGGTKLTVL |
| 1072 | F6A_LC_L83S | artificial | EVQLVESGGGLVQPGGSLKLSCAASGFTFNIYAMNWVRQAPGKGLEWVARIRSKYNNY |
| | -VH | | ATYYADSVKSRFTISRDDSKNTAYLQMNNLKTEDTAVYYCVRHGNFGNSYVSFFAYWG |
| | | | QGTLVTVSS |
| 1073 | F6A_LC_L83S | artificial | QTVVTQEPSLTVSPGGTVTLTCGSSTGAVTSGYYPNWVQQKPGQAPRGLIGGTKFLAP |
| | -VL | | GTPARFSGSLSGGKAALTLSGVQPEDEAEYYCALWYSNRWVFGGGTKLTVL |
| 1074 | F6A_LC_Q46E | artificial | EVQLVESGGGLVQPGGSLKLSCAASGFTFNIYAMNWVRQAPGKGLEWVARIRSKYNNY |
| | -VH | | ATYYADSVKSRFTISRDDSKNTAYLQMNNLKTEDTAVYYCVRHGNFGNSYVSFFAYWG |
| | | | QGTLVTVSS |
| 1075 | F6A_LC_Q46E | artificial | QTVVTQEPSLTVSPGGTVTLTCGSSTGAVTSGYYPNWVQEKPGQAPRGLIGGTKFLAP |
| | -VL | | GTPARFSGSLLGGKAALTLSGVQPEDEAEYYCALWYSNRWVFGGGTKLTVL |
| 1076 | F6A_LC_Q46K | artificial | EVQLVESGGGLVQPGGSLKLSCAASGFTFNIYAMNWVRQAPGKGLEWVARIRSKYNNY |
| | -VH | | ATYYADSVKSRFTISRDDSKNTAYLQMNNLKTEDTAVYYCVRHGNFGNSYVSFFAYWG |
| | | | QGTLVTVSS |
| 1077 | F6A_LC_Q46K | artificial | QTVVTQEPSLTVSPGGTVTLTCGSSTGAVTSGYYPNWVQKKPGQAPRGLIGGTKFLAP |
| | -VL | | GTPARFSGSLLGGKAALTLSGVQPEDEAEYYCALWYSNRWVFGGGTKLTVL |
| 1078 | F6A_LC_A107L- | artificial | IYAMN |
| | HCDR1 | | |
| 1079 | F6A_LC_A107L- | artificial | RIRSKYNNYATYYADSVKS |
| | HCDR2 | | |
| 1080 | F6A_LC_A107L- | artificial | HGNFGNSYVSFFAY |
| | HCDR3 | | |
| 1081 | F6A_LC_A107L- | artificial | GSSTGAVTSGYYPN |
| | LCDR1 | | |
| 1082 | F6A_LC_A107L- | artificial | GTKFLAP |
| | LCDR2 | | |
| 1083 | F6A_LC_A107L- | artificial | LLWYSNRWV |
| | LCDR3 | | |
| 1084 | F6A_LC_A107L- | artificial | EVQLVESGGGLVQPGGSLKLSCAASGFTFNIYAMNWVRQAPGKGLEWVARIRSKYNNY |
| | VH | | ATYYADSVKSRFTISRDDSKNTAYLQMNNLKTEDTAVYYCVRHGNFGNSYVSFFAYWG |
| | | | QGTLVTVSS |
| 1085 | F6A_LC_A107L- | artificial | QTVVTQEPSLTVSPGGTVTLTCGSSTGAVTSGYYPNWVQQKPGQAPRGLIGGTKFLAP |
| | VL | | GTPARFSGSLLGGKAALTLSGVQPEDEAEYYCLLWYSNRWVFGGGTKLTVL |
| 1086 | F6A_LC_V44I | artificial | EVQLVESGGGLVQPGGSLKLSCAASGFTFNIYAMNWVRQAPGKGLEWVARIRSKYNNY |
| | -VH | | ATYYADSVKSRFTISRDDSKNTAYLQMNNLKTEDTAVYYCVRHGNFGNSYVSFFAYWG |
| | | | QGTLVTVSS |
| 1087 | F6A_LC_V44I | artificial | QTVVTQEPSLTVSPGGTVTLTCGSSTGAVTSGYYPNWIQQKPGQAPRGLIGGTKFLAP |
| | -VL | | GTPARFSGSLLGGKAALTLSGVQPEDEAEYYCALWYSNRWVFGGGTKLTVL |
| 1088 | F6A_LC_V44L | artificial | EVQLVESGGGLVQPGGSLKLSCAASGFTFNIYAMNWVRQAPGKGLEWVARIRSKYNNY |
| | -VH | | ATYYADSVKSRFTISRDDSKNTAYLQMNNLKTEDTAVYYCVRHGNFGNSYVSFFAYWG |
| | | | QGTLVTVSS |
| 1089 | F6A_LC_V44L | artificial | QTVVTQEPSLTVSPGGTVTLTCGSSTGAVTSGYYPNWLQQKPGQAPRGLIGGTKFLAP |
| | -VL | | GTPARFSGSLLGGKAALTLSGVQPEDEAEYYCALWYSNRWVFGGGTKLTVL |
| 1090 | F6A_LC_V44M | artificial | EVQLVESGGGLVQPGGSLKLSCAASGFTFNIYAMNWVRQAPGKGLEWVARIRSKYNNY |
| | -VH | | ATYYADSVKSRFTISRDDSKNTAYLQMNNLKTEDTAVYYCVRHGNFGNSYVSFFAYWG |
| | | | QGTLVTVSS |
| 1091 | F6A_LC_V44M | artificial | QTVVTQEPSLTVSPGGTVTLTCGSSTGAVTSGYYPNWMQQKPGQAPRGLIGGTKFLAP |
| | -VL | | GTPARFSGSLLGGKAALTLSGVQPEDEAEYYCALWYSNRWVFGGGTKLTVL |
| 1092 | F6A_LC_W109Y- | artificial | IYAMN |
| | HCDR1 | | |
| 1093 | F6A_LC_W109Y- | artificial | RIRSKYNNYATYYADSVKS |
| | HCDR2 | | |
| 1094 | F6A_LC_W109Y- | artificial | HGNFGNSYVSFFAY |
| | HCDR3 | | |
| 1095 | F6A_LC_W109Y- | artificial | GSSTGAVTSGYYPN |
| | LCDR1 | | |
| 1096 | F6A_LC_W109Y- | artificial | GTKFLAP |
| | LCDR2 | | |
| 1097 | F6A_LC_W109Y- | artificial | ALYYSNRWV |
| | LCDR3 | | |
| 1098 | F6A_LC_W109Y- | artificial | EVQLVESGGGLVQPGGSLKLSCAASGFTFNIYAMNWVRQAPGKGLEWVARIRSKYNNY |
| | VH | | ATYYADSVKSRFTISRDDSKNTAYLQMNNLKTEDTAVYYCVRHGNFGNSYVSFFAYWG |
| | | | QGTLVTVSS |
| 1099 | F6A_LC_W109Y- | artificial | QTVVTQEPSLTVSPGGTVTLTCGSSTGAVTSGYYPNWVQQKPGQAPRGLIGGTKFLAP |
| | VL | | GTPARFSGSLLGGKAALTLSGVQPEDEAEYYCALYYSNRWVFGGGTKLTVL |
| 1100 | F7O-HCDR1 | artificial | VYAMN |
| 1101 | F7O-HCDR2 | artificial | RIRSKYNNYATYYADSVKK |
| 1102 | F7O-HCDR3 | artificial | HGNFGNSYISWWAY |
| 1103 | F7O-LCDR1 | artificial | GSSTGAVTSGYYPN |
| 1104 | F7O-LCDR2 | artificial | GTKFLAP |
| 1105 | F7O-LCDR3 | artificial | ALWYSNRWV |
| 1106 | F7O-VH | artificial | EVQLVESGGGLVQPGGSLKLSCAASGFTFNVYAMNWVRQAPGKGLEWVARIRSKYNNY |
| | | | ATYYADSVKKRFTISRDDSKNTAYLQMNNLKTEDTAVYYCVRHGNFGNSYISWWAYWG |
| | | | QGTLVTVSS |
| 1107 | F7O-VL | artificial | QTVVTQEPSLTVSPGGTVTLTCGSSTGAVTSGYYPNWVQQKPGQAPRGLIGGTKFLAP |
| | | | GTPARFSGSLLGGKAALTLSGVQPEDEAEYYCALWYSNRWVFGGGTKLTVL |
| 1108 | F7O_HC_A89I | artificial | EVQLVESGGGLVQPGGSLKLSCAASGFTFNVYAMNWVRQAPGKGLEWVARIRSKYNNY |
| | -VH | | ATYYADSVKKRFTISRDDSKNTIYLQMNNLKTEDTAVYYCVRHGNFGNSYISWWAYWG |
| | | | QGTLVTVSS |
| 1109 | F7O_HC_A89I | artificial | QTVVTQEPSLTVSPGGTVTLTCGSSTGAVTSGYYPNWVQQKPGQAPRGLIGGTKFLAP |
| | -VL | | GTPARFSGSLLGGKAALTLSGVQPEDEAEYYCALWYSNRWVFGGGTKLTVL |
| 1110 | F7O_HC_A89L | artificial | EVQLVESGGGLVQPGGSLKLSCAASGFTFNVYAMNWVRQAPGKGLEWVARIRSKYNNY |
| | -VH | | ATYYADSVKKRFTISRDDSKNTLYLQMNNLKTEDTAVYYCVRHGNFGNSYISWWAYWG |
| | | | QGTLVTVSS |
| 1111 | F7O_HC_A89L | artificial | QTVVTQEPSLTVSPGGTVTLTCGSSTGAVTSGYYPNWVQQKPGQAPRGLIGGTKFLAP |
| | -VL | | GTPARFSGSLLGGKAALTLSGVQPEDEAEYYCALWYSNRWVFGGGTKLTVL |
| 1112 | F7O_HC_A89T | artificial | EVQLVESGGGLVQPGGSLKLSCAASGFTFNVYAMNWVRQAPGKGLEWVARIRSKYNNY |
| | -VH | | ATYYADSVKKRFTISRDDSKNTTYLQMNNLKTEDTAVYYCVRHGNFGNSYISWWAYWG |
| | | | QGTLVTVSS |
| 1113 | F7O_HC_A89T | artificial | QTVVTQEPSLTVSPGGTVTLTCGSSTGAVTSGYYPNWVQQKPGQAPRGLIGGTKFLAP |
| | -VL | | GTPARFSGSLLGGKAALTLSGVQPEDEAEYYCALWYSNRWVFGGGTKLTVL |
| 1114 | F7O_HC_A89V | artificial | EVQLVESGGGLVQPGGSLKLSCAASGFTFNVYAMNWVRQAPGKGLEWVARIRSKYNNY |
| | -VH | | ATYYADSVKKRFTISRDDSKNTVYLQMNNLKTEDTAVYYCVRHGNFGNSYISWWAYWG |
| | | | QGTLVTVSS |
| 1115 | F7O_HC_A89V | artificial | QTVVTQEPSLTVSPGGTVTLTCGSSTGAVTSGYYPNWVQQKPGQAPRGLIGGTKFLAP |
| | -VL | | GTPARFSGSLLGGKAALTLSGVQPEDEAEYYCALWYSNRWVFGGGTKLTVL |
| 1116 | F7O_HC_L52M | artificial | EVQLVESGGGLVQPGGSLKLSCAASGFTFNVYAMNWVRQAPGKGMEWVARIRSKYNNY |
| | -VH | | ATYYADSVKKRFTISRDDSKNTAYLQMNNLKTEDTAVYYCVRHGNFGNSYISWWAYWG |
| | | | QGTLVTVSS |
| 1117 | F7O_HC_L52M | artificial | QTVVTQEPSLTVSPGGTVTLTCGSSTGAVTSGYYPNWVQQKPGQAPRGLIGGTKFLAP |
| | -VL | | GTPARFSGSLLGGKAALTLSGVQPEDEAEYYCALWYSNRWVFGGGTKLTVL |
| 1118 | F7O_HC_L52M_ | artificial | EVQLVESGGGLVQPGGSLKLSCAASGFTFNVYAMNWVRQAPGKGMEWVARIRSKYNNY |
| | LC_L21I_ | | ATYYADSVKKRFTISRDDSKNTAYLQMNNLKTEDTAVYYCVRHGNFGNSYISWWAYWG |
| | Q46K_G141S- | | QGTLVTVSS |
| | VH | | |
| 1119 | F7O_HC_L52M_ | artificial | QTVVTQEPSLTVSPGGTVTITCGSSTGAVTSGYYPNWVQKKPGQAPRGLIGGTKFLAP |
| | LC_L21I_ | | GTPARFSGSLLGGKAALTLSGVQPEDEAEYYCALWYSNRWVFGSGTKLTVL |
| | Q46K_G141S- | | |
| | VL | | |
| 1120 | F7O_HC_L52M_ | artificial | EVQLVESGGGLVQPGGSLKLSCAASGFTFNVYAMNWVRQAPGKGMEWVARIRSKYNNY |
| | LC_Q46K- | | ATYYADSVKKRFTISRDDSKNTAYLQMNNLKTEDTAVYYCVRHGNFGNSYISWWAYWG |
| | VH | | QGTLVTVSS |
| 1121 | F7O_HC_L52M_ | artificial | QTVVTQEPSLTVSPGGTVTLTCGSSTGAVTSGYYPNWVQKKPGQAPRGLIGGTKFLAP |
| | LC_Q46K- | | GTPARFSGSLLGGKAALTLSGVQPEDEAEYYCALWYSNRWVFGGGTKLTVL |
| | VL | | |
| 1122 | F7O_HC_L52M_ | artificial | EVQLVESGGGLVQPGGSLKLSCAASGFTFNVYAMNWVRQAPGKGMEWVARIRSKYNNY |
| | LC_Q46K_ | | ATYYADSVKKRFTISRDDSKNTAYLQMNNLKTEDTAVYYCVRHGNFGNSYISWWAYWG |
| | G141S-VH | | QGTLVTVSS |
| 1123 | F7O_HC_L52M_ | artificial | QTVVTQEPSLTVSPGGTVTLTCGSSTGAVTSGYYPNWVQKKPGQAPRGLIGGTKFLAP |
| | LC_Q46K_ | | GTPARFSGSLLGGKAALTLSGVQPEDEAEYYCALWYSNRWVFGSGTKLTVL |
| | G141S-VL | | |
| 1124 | F7O_HC_L52V | artificial | EVQLVESGGGLVQPGGSLKLSCAASGFTFNVYAMNWVRQAPGKGVEWVARIRSKYNNY |
| | -VH | | ATYYADSVKKRFTISRDDSKNTAYLQMNNLKTEDTAVYYCVRHGNFGNSYISWWAYWG |
| | | | QGTLVTVSS |
| 1125 | F7O_HC_L52V | artificial | QTVVTQEPSLTVSPGGTVTLTCGSSTGAVTSGYYPNWVQQKPGQAPRGLIGGTKFLAP |
| | -VL | | GTPARFSGSLLGGKAALTLSGVQPEDEAEYYCALWYSNRWVFGGGTKLTVL |
| 1126 | F7O_HC_L52V_ | artificial | EVQLVESGGGLVQPGGSLKLSCAASGFTFNVYAMNWVRQAPGKGVEWVARIRSKYNNY |
| | LC_V44F- | | ATYYADSVKKRFTISRDDSKNTAYLQMNNLKTEDTAVYYCVRHGNFGNSYISWWAYWG |
| | VH | | QGTLVTVSS |
| 1127 | F7O_HC_L52V_ | artificial | QTVVTQEPSLTVSPGGTVTLTCGSSTGAVTSGYYPNWFQQKPGQAPRGLIGGTKFLAP |
| | LC_V44F- | | GTPARFSGSLLGGKAALTLSGVQPEDEAEYYCALWYSNRWVFGGGTKLTVL |
| | VL | | |
| 1128 | F7O_HC_L52V_ | artificial | EVQLVESGGGLVQPGGSLKLSCAASGFTFNVYAMNWVRQAPGKGVEWVARIRSKYNNY |
| | LC_V44Y- | | ATYYADSVKKRFTISRDDSKNTAYLQMNNLKTEDTAVYYCVRHGNFGNSYISWWAYWG |
| | VH | | QGTLVTVSS |
| 1129 | F7O_HC_L52V_ | artificial | QTVVTQEPSLTVSPGGTVTLTCGSSTGAVTSGYYPNWYQQKPGQAPRGLIGGTKFLAP |
| | LC_V44Y | | GTPARFSGSLLGGKAALTLSGVQPEDEAEYYCALWYSNRWVFGGGTKLTVL |
| | VL | | |
| 1130 | F7O_HC_M41F_ | artificial | VYAFN |
| | A89V- | | |
| | HCDR1 | | |
| 1131 | F7O_HC_M41F_ | artificial | RIRSKYNNYATYYADSVKK |
| | A89V- | | |
| | HCDR2 | | |
| 1132 | F7O_HC_M41F_ | artificial | HGNFGNSYISWWAY |
| | A89V-HCDR3 | | |
| 1133 | F7O_HC_M41F_ | artificial | GSSTGAVTSGYYPN |
| | A89V-LCDR1 | | |
| 1134 | F7O_HC_M41F_ | artificial | GTKFLAP |
| | A89V-LCDR2 | | |
| 1135 | F7O_HC_M41F_ | artificial | ALWYSNRWV |
| | A89V-LCDR3 | | |
| 1136 | F7O_HC_M41F_ | artificial | EVQLVESGGGLVQPGGSLKLSCAASGFTFNVYAFNWVRQAPGKGLEWVARIRSKYNNY |
| | A89V-VH | | ATYYADSVKKRFTISRDDSKNTVYLQMNNLKTEDTAVYYCVRHGNFGNSYISWWAYWG |
| | | | QGTLVTVSS |
| 1137 | F7O_HC_M41F_ | artificial | QTVVTQEPSLTVSPGGTVTLTCGSSTGAVTSGYYPNWVQQKPGQAPRGLIGGTKFLAP |
| | A89V-VL | | GTPARFSGSLLGGKAALTLSGVQPEDEAEYYCALWYSNRWVFGGGTKLTVL |
| 1138 | F7O_HC_M41I | artificial | VYAIN |
| | -HCDR1 | | |
| 1139 | F7O_HC_M41I- | artificial | RIRSKYNNYATYYADSVKK |
| | HCDR2 | | |
| 1140 | F7O_HC_M41I | artificial | HGNFGNSYISWWAY |
| | -HCDR3 | | |
| 1141 | F7O_HC_M41I | artificial | GSSTGAVTSGYYPN |
| | -LCDR1 | | |
| 1142 | F7O_HC_M41I | artificial | GTKFLAP |
| | -LCDR2 | | |
| 1143 | F7O_HC_M41I | artificial | ALWYSNRWV |
| | -LCDR3 | | |
| 1144 | F7O_HC_M41I | artificial | EVQLVESGGGLVQPGGSLKLSCAASGFTFNVYAINWVRQAPGKGLEWVARIRSKYNNY |
| | -VH | | ATYYADSVKKRFTISRDDSKNTAYLQMNNLKTEDTAVYYCVRHGNFGNSYISWWAYWG |
| | | | QGTLVTVSS |
| 1145 | F7O_HC_M41I | artificial | QTVVTQEPSLTVSPGGTVTLTCGSSTGAVTSGYYPNWVQQKPGQAPRGLIGGTKFLAP |
| | -VL | | GTPARFSGSLLGGKAALTLSGVQPEDEAEYYCALWYSNRWVFGGGTKLTVL |
| 1146 | F7O_HC_M41I_ | artificial | EVQLVESGGGLVQPGGSLKLSCAASGFTFNVYAINWVRQAPGKGLEWVARIRSKYNNY |
| | A89L-VH | | ATYYADSVKKRFTISRDDSKNTLYLQMNNLKTEDTAVYYCVRHGNFGNSYISWWAYWG |
| | | | QGTLVTVSS |
| 1147 | F7O_HC_M41I_ | artificial | QTVVTQEPSLTVSPGGTVTLTCGSSTGAVTSGYYPNWVQQKPGQAPRGLIGGTKFLAP |
| | A89L-VL | | GTPARFSGSLLGGKAALTLSGVQPEDEAEYYCALWYSNRWVFGGGTKLTVL |
| 1148 | F7O_HC_M41I_ | artificial | EVQLVESGGGLVQPGGSLKLSCAASGFTFNVYAINWVRQAPGKGLEWVARIRSKYNNY |
| | A89V-VH | | ATYYADSVKKRFTISRDDSKNTVYLQMNNLKTEDTAVYYCVRHGNFGNSYISWWAYWG |
| | | | QGTLVTVSS |
| 1149 | F7O_HC_M41I_ | artificial | QTVVTQEPSLTVSPGGTVTLTCGSSTGAVTSGYYPNWVQQKPGQAPRGLIGGTKFLAP |
| | A89V-VL | | GTPARFSGSLLGGKAALTLSGVQPEDEAEYYCALWYSNRWVFGGGTKLTVL |
| 1150 | F7O_HC_M41I_ | artificial | EVQLVESGGGLVQPGGSLKLSCAASGFTFNVYAINWVRQAPGKGLEWVARIRSKYNNY |
| | A89V_LC_ | | ATYYADSVKKRFTISRDDSKNTVYLQMNNLKTEDTAVYYCVRHGNFGNSYISWWAYWG |
| | L21I_Q46K_ | | QGTLVTVSS |
| | G141S-VH | | |
| 1151 | F7O_HC_M41I_ | artificial | QTVVTQEPSLTVSPGGTVTITCGSSTGAVTSGYYPNWVQKKPGQAPRGLIGGTKFLAP |
| | A89V_LC_ | | GTPARFSGSLLGGKAALTLSGVQPEDEAEYYCALWYSNRWVFGSGTKLTVL |
| | L21I_Q46K_ | | |
| | G141S-VL | | |
| 1152 | F7O_HC_M41I_ | artificial | EVQLVESGGGLVQPGGSLKLSCAASGFTFNVYAINWVRQAPGKGLEWVARIRSKYNNY |
| | A89V_LC_ | | ATYYADSVKKRFTISRDDSKNTVYLQMNNLKTEDTAVYYCVRHGNFGNSYISWWAYWG |
| | Q46K-VH | | QGTLVTVSS |
| 1153 | F7O_HC_M41I_ | artificial | QTVVTQEPSLTVSPGGTVTLTCGSSTGAVTSGYYPNWVQKKPGQAPRGLIGGTKFLAP |
| | A89V_LC_ | | GTPARFSGSLLGGKAALTLSGVQPEDEAEYYCALWYSNRWVFGGGTKLTVL |
| | Q46K-VL | | |
| 1154 | F7O_HC_M41I_ | artificial | EVQLVESGGGLVQPGGSLKLSCAASGFTFNVYAINWVRQAPGKGVEWVARIRSKYNNY |
| | L52V_A89V_ | | ATYYADSVKKRFTISRDDSKNTVYLQMNNLKTEDTAVYYCVRHGNFGNSYISWWAYWG |
| | LC_L21I_ | | QGTLVTVSS |
| | Q46K_G141S_ | | |
| | V44F-VH | | |
| 1155 | F7O_HC_M41I_ | artificial | QTVVTQEPSLTVSPGGTVTITCGSSTGAVTSGYYPNWFQKKPGQAPRGLIGGTKFLAP |
| | L52V_A89V_ | | GTPARFSGSLLGGKAALTLSGVQPEDEAEYYCALWYSNRWVFGSGTKLTVL |
| | LC_L21I_ | | |
| | Q46K_G141S_ | | |
| | V44F-VL | | |
| 1156 | F7O_HC_M41I_ | artificial | EVQLVESGGGLVQPGGSLKLSCAASGFTFNVYAINWVRQAPGKGLEWVARIRSKYNNY |
| | LC_L21I_ | | ATYYADSVKKRFTISRDDSKNTAYLQMNNLKTEDTAVYYCVRHGNFGNSYISWWAYWG |
| | Q46K_G141S- | | QGTLVTVSS |
| | VH | | |
| 1157 | F7O_HC_M41I_ | artificial | QTVVTQEPSLTVSPGGTVTITCGSSTGAVTSGYYPNWVQKKPGQAPRGLIGGTKFLAP |
| | LC_L21I_ | | GTPARFSGSLLGGKAALTLSGVQPEDEAEYYCALWYSNRWVFGSGTKLTVL |
| | Q46K_G141S- | | |
| | VL | | |
| 1158 | F7O_HC_M41I_ | artificial | EVQLVESGGGLVQPGGSLKLSCAASGFTFNVYAINWVREAPGKGLEWVARIRSKYNNY |
| | Q46E_A89V_ | | ATYYADSVKKRFTISRDDSKNTVYLQMNNLKTEDTAVYYCVRHGNFGNSYISWWAYWG |
| | LC_L21I_ | | QGTLVTVSS |
| | Q46K_G141S- | | |
| | VH | | |
| 1159 | F7O_HC_M41I_ | artificial | QTVVTQEPSLTVSPGGTVTITCGSSTGAVTSGYYPNWVQKKPGQAPRGLIGGTKFLAP |
| | Q46E_A89V_ | | GTPARFSGSLLGGKAALTLSGVQPEDEAEYYCALWYSNRWVFGSGTKLTVL |
| | LC_L21I_ | | |
| | Q46K_G141S- | | |
| | VL | | |
| 1160 | F7O_HC_M41V | artificial | VYAVN |
| | -HCDR1 | | |
| 1161 | F7O_HC_M41V | artificial | RIRSKYNNYATYYADSVKK |
| | -HCDR2 | | |
| 1162 | F7O_HC_M41V | artificial | HGNFGNSYISWWAY |
| | -HCDR3 | | |
| 1163 | F7O_HC_M41V | artificial | GSSTGAVTSGYYPN |
| | -LCDR1 | | |
| 1164 | F7O_HC_M41V | artificial | GTKFLAP |
| | -LCDR2 | | |
| 1165 | F7O_HC_M41V | artificial | ALWYSNRWV |
| | -LCDR3 | | |
| 1166 | F7O_HC_M41V | artificial | EVQLVESGGGLVQPGGSLKLSCAASGFTFNVYAVNWVRQAPGKGLEWVARIRSKYNNY |
| | -VH | | ATYYADSVKKRFTISRDDSKNTAYLQMNNLKTEDTAVYYCVRHGNFGNSYISWWAYWG |
| | | | QGTLVTVSS |
| 1167 | F7O_HC_M41V | artificial | QTVVTQEPSLTVSPGGTVTLTCGSSTGAVTSGYYPNWVQQKPGQAPRGLIGGTKFLAP |
| | -VL | | GTPARFSGSLLGGKAALTLSGVQPEDEAEYYCALWYSNRWVFGGGTKLTVL |
| 1168 | F7O_HC_Q46D | artificial | EVQLVESGGGLVQPGGSLKLSCAASGFTFNVYAMNWVRDAPGKGLEWVARIRSKYNNY |
| | -VH | | ATYYADSVKKRFTISRDDSKNTAYLQMNNLKTEDTAVYYCVRHGNFGNSYISWWAYWG |
| | | | QGTLVTVSS |
| 1169 | F7O_HC_Q46D | artificial | QTVVTQEPSLTVSPGGTVTLTCGSSTGAVTSGYYPNWVQQKPGQAPRGLIGGTKFLAP |
| | -VL | | GTPARFSGSLLGGKAALTLSGVQPEDEAEYYCALWYSNRWVFGGGTKLTVL |
| 1170 | F7O_HC_Q46D_ | artificial | EVQLVESGGGLVQPGGSLKLSCAASGFTFNVYAMNWVRDAPGKGLEWVARIRSKYNNY |
| | LC_L21I_ | | ATYYADSVKKRFTISRDDSKNTAYLQMNNLKTEDTAVYYCVRHGNFGNSYISWWAYWG |
| | Q46K_G141S- | | QGTLVTVSS |
| | VH | | |
| 1171 | F7O_HC_Q46D_ | artificial | QTVVTQEPSLTVSPGGTVTITCGSSTGAVTSGYYPNWVQKKPGQAPRGLIGGTKFLAP |
| | LC_L21I_ | | GTPARFSGSLLGGKAALTLSGVQPEDEAEYYCALWYSNRWVFGSGTKLTVL |
| | Q46K_G141S- | | |
| | VL | | |
| 1172 | F7O_HC_Q46D_ | artificial | EVQLVESGGGLVQPGGSLKLSCAASGFTFNVYAMNWVRDAPGKGLEWVARIRSKYNNY |
| | LC_L21I_ | | ATYYADSVKKRFTISRDDSKNTAYLQMNNLKTEDTAVYYCVRHGNFGNSYISWWAYWG |
| | Q46R_G141S- | | QGTLVTVSS |
| | VH | | |
| 1173 | F7O_HC_Q46D_ | artificial | QTVVTQEPSLTVSPGGTVTITCGSSTGAVTSGYYPNWVQRKPGQAPRGLIGGTKFLAP |
| | LC_L21I_ | | GTPARFSGSLLGGKAALTLSGVQPEDEAEYYCALWYSNRWVFGSGTKLTVL |
| | Q46R_G141S- | | |
| | VL | | |
| 1174 | F7O_HC_Q46D_ | artificial | EVQLVESGGGLVQPGGSLKLSCAASGFTFNVYAMNWVRDAPGKGLEWVARIRSKYNNY |
| | LC_Q46K- | | ATYYADSVKKRFTISRDDSKNTAYLQMNNLKTEDTAVYYCVRHGNFGNSYISWWAYWG |
| | VH | | QGTLVTVSS |
| 1175 | F7O_HC_Q46D_ | artificial | QTVVTQEPSLTVSPGGTVTLTCGSSTGAVTSGYYPNWVQKKPGQAPRGLIGGTKFLAP |
| | LC_Q46K- | | GTPARFSGSLLGGKAALTLSGVQPEDEAEYYCALWYSNRWVFGGGTKLTVL |
| | VL | | |
| 1176 | F7O_HC_Q46D_ | artificial | EVQLVESGGGLVQPGGSLKLSCAASGFTFNVYAMNWVRDAPGKGLEWVARIRSKYNNY |
| | LC_Q46K_G1 | | ATYYADSVKKRFTISRDDSKNTAYLQMNNLKTEDTAVYYCVRHGNFGNSYISWWAYWG |
| | 41S-VH | | QGTLVTVSS |
| 1177 | F7O_HC_Q46D_ | artificial | QTVVTQEPSLTVSPGGTVTLTCGSSTGAVTSGYYPNWVQKKPGQAPRGLIGGTKFLAP |
| | LC_Q46K_ | | GTPARFSGSLLGGKAALTLSGVQPEDEAEYYCALWYSNRWVFGSGTKLTVL |
| | G141S-VL | | |
| 1178 | F7O_HC_Q46D_ | artificial | EVQLVESGGGLVQPGGSLKLSCAASGFTFNVYAMNWVRDAPGKGLEWVARIRSKYNNY |
| | LC_Q46R- | | ATYYADSVKKRFTISRDDSKNTAYLQMNNLKTEDTAVYYCVRHGNFGNSYISWWAYWG |
| | VH | | QGTLVTVSS |
| 1179 | F7O_HC_Q46D_ | artificial | QTVVTQEPSLTVSPGGTVTLTCGSSTGAVTSGYYPNWVQRKPGQAPRGLIGGTKFLAP |
| | LC_Q46R- | | GTPARFSGSLLGGKAALTLSGVQPEDEAEYYCALWYSNRWVFGGGTKLTVL |
| | VL | | |
| 1180 | F7O_HC_Q46D_ | artificial | EVQLVESGGGLVQPGGSLKLSCAASGFTFNVYAMNWVRDAPGKGLEWVARIRSKYNNY |
| | LC_Q46R_ | | ATYYADSVKKRFTISRDDSKNTAYLQMNNLKTEDTAVYYCVRHGNFGNSYISWWAYWG |
| | G141S-VH | | QGTLVTVSS |
| 1181 | F7O_HC_Q46D_ | artificial | QTVVTQEPSLTVSPGGTVTLTCGSSTGAVTSGYYPNWVQRKPGQAPRGLIGGTKFLAP |
| | LC_Q46R_ | | GTPARFSGSLLGGKAALTLSGVQPEDEAEYYCALWYSNRWVFGSGTKLTVL |
| | G141S-VL | | |
| 1182 | F7O_HC_Q46E | artificial | EVQLVESGGGLVQPGGSLKLSCAASGFTFNVYAMNWVREAPGKGLEWVARIRSKYNNY |
| | -VH | | ATYYADSVKKRFTISRDDSKNTAYLQMNNLKTEDTAVYYCVRHGNFGNSYISWWAYWG |
| | | | QGTLVTVSS |
| 1183 | F7O_HC_Q46E | artificial | QTVVTQEPSLTVSPGGTVTLTCGSSTGAVTSGYYPNWVQQKPGQAPRGLIGGTKFLAP |
| | -VL | | GTPARFSGSLLGGKAALTLSGVQPEDEAEYYCALWYSNRWVFGGGTKLTVL |
| 1184 | F7O_HC_Q46E_ | artificial | EVQLVESGGGLVQPGGSLKLSCAASGFTFNVYAMNWVREAPGKGLEWVARIRSKYNNY |
| | LC_L21I_ | | ATYYADSVKKRFTISRDDSKNTAYLQMNNLKTEDTAVYYCVRHGNFGNSYISWWAYWG |
| | Q46K_G141S- | | QGTLVTVSS |
| | VH | | |
| 1185 | F7O_HC_Q46E_ | artificial | QTVVTQEPSLTVSPGGTVTITCGSSTGAVTSGYYPNWVQKKPGQAPRGLIGGTKFLAP |
| | LC_L21I_ | | GTPARFSGSLLGGKAALTLSGVQPEDEAEYYCALWYSNRWVFGSGTKLTVL |
| | Q46K_G141S- | | |
| | VL | | |
| 1186 | F7O_HC_Q46E_ | artificial | EVQLVESGGGLVQPGGSLKLSCAASGFTFNVYAMNWVREAPGKGLEWVARIRSKYNNY |
| | LC_L21I_ | | ATYYADSVKKRFTISRDDSKNTAYLQMNNLKTEDTAVYYCVRHGNFGNSYISWWAYWG |
| | Q46R_G141S- | | QGTLVTVSS |
| | VH | | |
| 1187 | F7O_HC_Q46E_ | artificial | QTVVTQEPSLTVSPGGTVTITCGSSTGAVTSGYYPNWVQRKPGQAPRGLIGGTKFLAP |
| | LC_L21I_ | | GTPARFSGSLLGGKAALTLSGVQPEDEAEYYCALWYSNRWVFGSGTKLTVL |
| | Q46R_G141S- | | |
| | VL | | |
| 1188 | F7O_HC_Q46E_ | artificial | EVQLVESGGGLVQPGGSLKLSCAASGFTFNVYAMNWVREAPGKGLEWVARIRSKYNNY |
| | LC_Q46K- | | ATYYADSVKKRFTISRDDSKNTAYLQMNNLKTEDTAVYYCVRHGNFGNSYISWWAYWG |
| | VH | | QGTLVTVSS |
| 1189 | F7O_HC_Q46E_ | artificial | QTVVTQEPSLTVSPGGTVTLTCGSSTGAVTSGYYPNWVQKKPGQAPRGLIGGTKFLAP |
| | LC_Q46K- | | GTPARFSGSLLGGKAALTLSGVQPEDEAEYYCALWYSNRWVFGGGTKLTVL |
| | VL | | |
| 1190 | F7O_HC_Q46E_ | artificial | EVQLVESGGGLVQPGGSLKLSCAASGFTFNVYAMNWVREAPGKGLEWVARIRSKYNNY |
| | LC_Q46K_ | | ATYYADSVKKRFTISRDDSKNTAYLQMNNLKTEDTAVYYCVRHGNFGNSYISWWAYWG |
| | G141S-VH | | QGTLVTVSS |
| 1191 | F7O_HC_Q46E_ | artificial | QTVVTQEPSLTVSPGGTVTLTCGSSTGAVTSGYYPNWVQKKPGQAPRGLIGGTKFLAP |
| | LC_Q46K_ | | GTPARFSGSLLGGKAALTLSGVQPEDEAEYYCALWYSNRWVFGSGTKLTVL |
| | G141S-VL | | |
| 1192 | F7O_HC_Q46E_ | artificial | EVQLVESGGGLVQPGGSLKLSCAASGFTFNVYAMNWVREAPGKGLEWVARIRSKYNNY |
| | LC_Q46R- | | ATYYADSVKKRFTISRDDSKNTAYLQMNNLKTEDTAVYYCVRHGNFGNSYISWWAYWG |
| | VH | | QGTLVTVSS |
| 1193 | F7O_HC_Q46E_ | artificial | QTVVTQEPSLTVSPGGTVTLTCGSSTGAVTSGYYPNWVQRKPGQAPRGLIGGTKFLAP |
| | LC_Q46R- | | GTPARFSGSLLGGKAALTLSGVQPEDEAEYYCALWYSNRWVFGGGTKLTVL |
| | VL | | |
| 1194 | F7O_HC_Q46E_ | artificial | EVQLVESGGGLVQPGGSLKLSCAASGFTFNVYAMNWVREAPGKGLEWVARIRSKYNNY |
| | LC_Q46R_ | | ATYYADSVKKRFTISRDDSKNTAYLQMNNLKTEDTAVYYCVRHGNFGNSYISWWAYWG |
| | G141S-VH | | QGTLVTVSS |
| 1195 | F7O_HC_Q46E_ | artificial | QTVVTQEPSLTVSPGGTVTLTCGSSTGAVTSGYYPNWVQRKPGQAPRGLIGGTKFLAP |
| | LC_Q46R_ | | GTPARFSGSLLGGKAALTLSGVQPEDEAEYYCALWYSNRWVFGSGTKLTVL |
| | G141S-VL | | |
| 1196 | F7O_HC_Q46K | artificial | EVQLVESGGGLVQPGGSLKLSCAASGFTFNVYAMNWVRKAPGKGLEWVARIRSKYNNY |
| | -VH | | ATYYADSVKKRFTISRDDSKNTAYLQMNNLKTEDTAVYYCVRHGNFGNSYISWWAYWG |
| | | | QGTLVTVSS |
| 1197 | F7O_HC_Q46K | artificial | QTVVTQEPSLTVSPGGTVTLTCGSSTGAVTSGYYPNWVQQKPGQAPRGLIGGTKFLAP |
| | -VL | | GTPARFSGSLLGGKAALTLSGVQPEDEAEYYCALWYSNRWVFGGGTKLTVL |
| 1198 | F7O_HC_Q46R | artificial | EVQLVESGGGLVQPGGSLKLSCAASGFTFNVYAMNWVRRAPGKGLEWVARIRSKYNNY |
| | -VH | | ATYYADSVKKRFTISRDDSKNTAYLQMNNLKTEDTAVYYCVRHGNFGNSYISWWAYWG |
| | | | QGTLVTVSS |
| 1199 | F7O_HC_Q46R | artificial | QTVVTQEPSLTVSPGGTVTLTCGSSTGAVTSGYYPNWVQQKPGQAPRGLIGGTKFLAP |
| | -VL | | GTPARFSGSLLGGKAALTLSGVQPEDEAEYYCALWYSNRWVFGGGTKLTVL |
| 1200 | F7O_HC_V107A- | artificial | EVQLVESGGGLVQPGGSLKLSCAASGFTFNVYAMNWVRQAPGKGLEWVARIRSKYNNY |
| | VH | | ATYYADSVKKRFTISRDDSKNTAYLQMNNLKTEDTAVYYCARHGNFGNSYISWWAYWG |
| | | | QGTLVTVSS |
| 1201 | F7O_HC_V107A- | artificial | QTVVTQEPSLTVSPGGTVTLTCGSSTGAVTSGYYPNWVQQKPGQAPRGLIGGTKFLAP |
| | VL | | GTPARFSGSLLGGKAALTLSGVQPEDEAEYYCALWYSNRWVFGGGTKLTVL |
| 1202 | F7O_HC_V107L- | artificial | EVQLVESGGGLVQPGGSLKLSCAASGFTFNVYAMNWVRQAPGKGLEWVARIRSKYNNY |
| | VH | | ATYYADSVKKRFTISRDDSKNTAYLQMNNLKTEDTAVYYCLRHGNFGNSYISWWAYWG |
| | | | QGTLVTVSS |
| 1203 | F7O_HC_V107L- | artificial | QTVVTQEPSLTVSPGGTVTLTCGSSTGAVTSGYYPNWVQQKPGQAPRGLIGGTKFLAP |
| | VL | | GTPARFSGSLLGGKAALTLSGVQPEDEAEYYCALWYSNRWVFGGGTKLTVL |
| 1204 | F7O_HC_W136F- | artificial | VYAMN |
| | HCDR1 | | |
| 1205 | F7O_HC_W136F- | artificial | RIRSKYNNYATYYADSVKK |
| | HCDR2 | | |
| 1206 | F7O_HC_W136F- | artificial | HGNFGNSYISWFAY |
| | HCDR3 | | |
| 1207 | F7O_HC_W136F- | artificial | GSSTGAVTSGYYPN |
| | LCDR1 | | |
| 1208 | F7O_HC_W136F- | artificial | GTKFLAP |
| | LCDR2 | | |
| 1209 | F7O_HC_W136F- | artificial | ALWYSNRWV |
| | LCDR3 | | |
| 1210 | F7O_HC_W136F- | artificial | EVQLVESGGGLVQPGGSLKLSCAASGFTFNVYAMNWVRQAPGKGLEWVARIRSKYNNY |
| | VH | | ATYYADSVKKRFTISRDDSKNTAYLQMNNLKTEDTAVYYCVRHGNFGNSYISWFAYWG |
| | | | QGTLVTVSS |
| 1211 | F7O_HC_W136F- | artificial | QTVVTQEPSLTVSPGGTVTLTCGSSTGAVTSGYYPNWVQQKPGQAPRGLIGGTKFLAP |
| | VL | | GTPARFSGSLLGGKAALTLSGVQPEDEAEYYCALWYSNRWVFGGGTKLTVL |
| 1212 | F7O_HC_W136F_ | artificial | EVQLVESGGGLVQPGGSLKLSCAASGFTFNVYAMNWVRQAPGKGLEWVARIRSKYNNY |
| | LC_V44I- | | ATYYADSVKKRFTISRDDSKNTAYLQMNNLKTEDTAVYYCVRHGNFGNSYISWFAYWG |
| | VH | | QGTLVTVSS |
| 1213 | F7O_HC_W136F_ | artificial | QTVVTQEPSLTVSPGGTVTLTCGSSTGAVTSGYYPNWIQQKPGQAPRGLIGGTKFLAP |
| | LC_V44I- | | GTPARFSGSLLGGKAALTLSGVQPEDEAEYYCALWYSNRWVFGGGTKLTVL |
| | VL | | |
| 1214 | F7O_HC_W136F_ | artificial | EVQLVESGGGLVQPGGSLKLSCAASGFTFNVYAMNWVRQAPGKGLEWVARIRSKYNNY |
| | LC_V44L- | | ATYYADSVKKRFTISRDDSKNTAYLQMNNLKTEDTAVYYCVRHGNFGNSYISWFAYWG |
| | VH | | QGTLVTVSS |
| 1215 | F7O_HC_W136F_ | artificial | QTVVTQEPSLTVSPGGTVTLTCGSSTGAVTSGYYPNWLQQKPGQAPRGLIGGTKFLAP |
| | LC_V44L- | | GTPARFSGSLLGGKAALTLSGVQPEDEAEYYCALWYSNRWVFGGGTKLTVL |
| | VL | | |
| 1216 | F7O_I2E- | artificial | VYAIN |
| | HCDR1 | | |
| 1217 | F7O_I2E- | artificial | RIRSKYNNYATYYADAVKK |
| | HCDR2 | | |
| 1218 | F7O_I2E- | artificial | AGNFGSSYISWWAY |
| | HCDR3 | | |
| 1219 | F7O_I2E- | artificial | GSSTGAVTSGYYPN |
| | LCDR1 | | |
| 1220 | F7O_I2E- | artificial | GTKFLAP |
| | LCDR2 | | |
| 1221 | F7O_I2E- | artificial | ALWYSNRWV |
| | LCDR3 | | |
| 1222 | F7O_I2E- | artificial | EVQLVESGGGLVQPGGSLKLSCAASGFTFNVYAINWVRQAPGKGLEWVARIRSKYNNY |
| | VH | | ATYYADAVKKRFTISRDDSKNTVYLQMNNLKTEDTAVYYCARAGNFGSSYISWWAYWG |
| | | | QGTLVTVSS |
| 1223 | F7O_I2E- | artificial | QTVVTQEPSLTVSPGGTVTITCGSSTGAVTSGYYPNWVQKKPGQAPRGLIGGTKFLAP |
| | VL | | GTPARFSGSLSGGKAALTLSGVQPEDEAEYYCALWYSNRWVFGSGTKLTVL |
| 1224 | F7O_I2K- | artificial | VYAMN |
| | HCDR1 | | |
| 1225 | F7O_I2K- | artificial | RIRSKYNNYATYYAEAVKG |
| | HCDR2 | | |
| 1226 | F7O_I2K- | artificial | NENIGTSYISWWAY |
| | HCDR3 | | |
| 1227 | F7O_I2K- | artificial | GSSTGAVTSGYYPN |
| | LCDR1 | | |
| 1228 | F7O_I2K- | artificial | GTKFLAP |
| | LCDR2 | | |
| 1229 | F7O_I2K- | artificial | ALWYSNRWV |
| | LCDR3 | | |
| 1230 | F7O_I2K- | artificial | EVQLVESGGGLVQPGGSLKLSCAASGFTFNVYAMNWVRQAPGKGLEWVARIRSKYNNY |
| | VH | | ATYYAEAVKGRFTISRDDSKNTVYLQMNNLKTEDTAVYYCVRNENIGTSYISWWAYWG |
| | | | QGTLVTVSS |
| 1231 | F7O_I2K- | artificial | QTVVTQEPSLTVSPGGTVTITCGSSTGAVTSGYYPNWVQQKPGQAPRGLIGGTKFLAP |
| | VL | | GTPARFSGSLSGGKAALTLSGVQPEDEAEYYCALWYSNRWVFGSGTKLTVL |
| 1232 | F7O_I2L- | artificial | VYAMN |
| | HCDR1 | | |
| 1233 | F7O_I2L- | artificial | RIRSKYNNYATYYADAVKK |
| | HCDR2 | | |
| 1234 | F7O_I2L- | artificial | AGNFGSSYISWFAY |
| | HCDR3 | | |
| 1235 | F7O_I2L- | artificial | GSSTGAVTSGYYPN |
| | LCDR1 | | |
| 1236 | F7O_I2L- | artificial | GTKFLAP |
| | LCDR2 | | |
| 1237 | F7O_I2L- | artificial | ALYYSNRWV |
| | LCDR3 | | |
| 1238 | F7O_I2L- | artificial | EVQLVESGGGLVQPGGSLKLSCAASGFTFNVYAMNWVRQAPGKGMEWVARIRSKYNNY |
| | VH | | ATYYADAVKKRFTISRDDSKNTLYLQMNNLKTEDTAVYYCVRAGNFGSSYISWFAYWG |
| | | | QGTLVTVSS |
| 1239 | F7O_I2L- | artificial | QTVVTQEPSLTVSPGGTVTITCGSSTGAVTSGYYPNWIQKKPGQAPRGLIGGTKFLAP |
| | VL | | GTPARFSGSLEGGKAALTLSGVQPEDEAEYYCALYYSNRWVFGSGTKLTVL |
| 1240 | F7O_I2M- | artificial | VYAMN |
| | HCDR1 | | |
| 1241 | F7O_I2M- | artificial | RIRSKYNNYATYYADAVKK |
| | HCDR2 | | |
| 1242 | F7O_I2M- | artificial | AGNFGTSYISWWAY |
| | HCDR3 | | |
| 1243 | F7O_I2M- | artificial | GSSTGAVTSGYYPN |
| | LCDR1 | | |
| 1244 | F7O_I2M- | artificial | GTKFLAP |
| | LCDR2 | | |
| 1245 | F7O_I2M- | artificial | ALWYSNRWV |
| | LCDR3 | | |
| 1246 | F7O_I2M- | artificial | EVQLVESGGGLVQPGGSLKLSCAASGFTFNVYAMNWVRQAPGKGMEWVARIRSKYNNY |
| | VH | | ATYYADAVKKRFTISRDDSKNTLYLQMNNLKTEDTAVYYCVRAGNFGTSYISWWAYWG |
| | | | QGTLVTVSS |
| 1247 | F7O_I2M- | artificial | QTVVTQEPSLTVSPGGTVTITCGSSTGAVTSGYYPNWVQKKPGQAPRGLIGGTKFLAP |
| | VL | | GTPARFSGSLSGGKAALTLSGVQPEDEAEYYCALWYSNRWVFGSGTKLTVL |
| 1248 | F7O_I2M2- | artificial | VYAIN |
| | HCDR1 | | |
| 1249 | F7O_I2M2- | artificial | RIRSKYNNYATYYADAVKK |
| | HCDR2 | | |
| 1250 | F7O_I2M2- | artificial | NANFGTSYISWFAY |
| | HCDR3 | | |
| 1251 | F7O_I2M2- | artificial | GSSTGAVTSGYYPN |
| | LCDR1 | | |
| 1252 | F7O_I2M2- | artificial | GTKFLAP |
| | LCDR2 | | |
| 1253 | F7O_I2M2- | artificial | ALWYSNRWV |
| | LCDR3 | | |
| 1254 | F7O_I2M2_ | artificial | EVQLVESGGGLVQPGGSLKLSCAASGFTFNVYAINWVREAPGKGLEWVARIRSKYNNY |
| | VH | | ATYYADAVKKRFTISRDDSKNTAYLQMNNLKTEDTAVYYCVRNANFGTSYISWFAYWG |
| | | | QGTLVTVSS |
| 1255 | F7O_I2M2- | artificial | QTVVTQEPSLTVSPGGTVTLTCGSSTGAVTSGYYPNWVQKKPGQAPRGLIGGTKFLAP |
| | VL | | GTPARFSGSLLGGKAALTLSGVQPEDEAEYYCALWYSNRWVFGSGTKLTVL |
| 1256 | F7O_LC_G141S- | artificial | EVQLVESGGGLVQPGGSLKLSCAASGFTFNVYAMNWVRQAPGKGLEWVARIRSKYNNY |
| | VH | | ATYYADSVKKRFTISRDDSKNTAYLQMNNLKTEDTAVYYCVRHGNFGNSYISWWAYWG |
| | | | QGTLVTVSS |
| 1257 | F7O_LC_G141S- | artificial | QTVVTQEPSLTVSPGGTVTLTCGSSTGAVTSGYYPNWVQQKPGQAPRGLIGGTKFLAP |
| | VL | | GTPARFSGSLLGGKAALTLSGVQPEDEAEYYCALWYSNRWVFGSGTKLTVL |
| 1258 | F7O_LC_G141S_ | artificial | EVQLVESGGGLVQPGGSLKLSCAASGFTFNVYAMNWVRQAPGKGLEWVARIRSKYNNY |
| | L21I-VH | | ATYYADSVKKRFTISRDDSKNTAYLQMNNLKTEDTAVYYCVRHGNFGNSYISWWAYWG |
| | | | QGTLVTVSS |
| 1259 | F7O_LC_G141S_ | artificial | QTVVTQEPSLTVSPGGTVTITCGSSTGAVTSGYYPNWVQQKPGQAPRGLIGGTKFLAP |
| | L21I-VL | | GTPARFSGSLLGGKAALTLSGVQPEDEAEYYCALWYSNRWVFGSGTKLTVL |
| 1260 | F7O_LC_L83E | artificial | EVQLVESGGGLVQPGGSLKLSCAASGFTFNVYAMNWVRQAPGKGLEWVARIRSKYNNY |
| | -VH | | ATYYADSVKKRFTISRDDSKNTAYLQMNNLKTEDTAVYYCVRHGNFGNSYISWWAYWG |
| | | | QGTLVTVSS |
| 1261 | F7O_LC_L83E | artificial | QTVVTQEPSLTVSPGGTVTLTCGSSTGAVTSGYYPNWVQQKPGQAPRGLIGGTKFLAP |
| | -VL | | GTPARFSGSLEGGKAALTLSGVQPEDEAEYYCALWYSNRWVFGGGTKLTVL |
| 1262 | F7O_LC_L83S | artificial | EVQLVESGGGLVQPGGSLKLSCAASGFTFNVYAMNWVRQAPGKGLEWVARIRSKYNNY |
| | -VH | | ATYYADSVKKRFTISRDDSKNTAYLQMNNLKTEDTAVYYCVRHGNFGNSYISWWAYWG |
| | | | QGTLVTVSS |
| 1263 | F7O_LC_L83S | artificial | QTVVTQEPSLTVSPGGTVTLTCGSSTGAVTSGYYPNWVQQKPGQAPRGLIGGTKFLAP |
| | -VL | | GTPARFSGSLSGGKAALTLSGVQPEDEAEYYCALWYSNRWVFGGGTKLTVL |
| 1264 | F7O_LC_Q46E | artificial | EVQLVESGGGLVQPGGSLKLSCAASGFTFNVYAMNWVRQAPGKGLEWVARIRSKYNNY |
| | -VH | | ATYYADSVKKRFTISRDDSKNTAYLQMNNLKTEDTAVYYCVRHGNFGNSYISWWAYWG |
| | | | QGTLVTVSS |
| 1265 | F7O_LC_Q46E | artificial | QTVVTQEPSLTVSPGGTVTLTCGSSTGAVTSGYYPNWVQEKPGQAPRGLIGGTKFLAP |
| | -VL | | GTPARFSGSLLGGKAALTLSGVQPEDEAEYYCALWYSNRWVFGGGTKLTVL |
| 1266 | F7O_LC_Q46K | artificial | EVQLVESGGGLVQPGGSLKLSCAASGFTFNVYAMNWVRQAPGKGLEWVARIRSKYNNY |
| | -VH | | ATYYADSVKKRFTISRDDSKNTAYLQMNNLKTEDTAVYYCVRHGNFGNSYISWWAYWG |
| | | | QGTLVTVSS |
| 1267 | F7O_LC_Q46K | artificial | QTVVTQEPSLTVSPGGTVTLTCGSSTGAVTSGYYPNWVQKKPGQAPRGLIGGTKFLAP |
| | -VL | | GTPARFSGSLLGGKAALTLSGVQPEDEAEYYCALWYSNRWVFGGGTKLTVL |
| 1268 | F7O_LC_A107L- | artificial | VYAMN |
| | HCDR1 | | |
| 1269 | F7O_LC_A107L- | artificial | RIRSKYNNYATYYADSVKK |
| | HCDR2 | | |
| 1270 | F7O_LC_A107L- | artificial | HGNFGNSYISWWAY |
| | HCDR3 | | |
| 1271 | F7O_LC_A107L- | artificial | GSSTGAVTSGYYPN |
| | LCDR1 | | |
| 1272 | F7O_LC_A107L- | artificial | GTKFLAP |
| | LCDR2 | | |
| 1273 | F7O_LC_A107L- | artificial | LLWYSNRWV |
| | LCDR3 | | |
| 1274 | F7O_LC_A107L- | artificial | EVQLVESGGGLVQPGGSLKLSCAASGFTFNVYAMNWVRQAPGKGLEWVARIRSKYNNY |
| | VH | | ATYYADSVKKRFTISRDDSKNTAYLQMNNLKTEDTAVYYCVRHGNFGNSYISWWAYWG |
| | | | QGTLVTVSS |
| 1275 | F7O_LC_A107L- | artificial | QTVVTQEPSLTVSPGGTVTLTCGSSTGAVTSGYYPNWVQQKPGQAPRGLIGGTKFLAP |
| | VL | | GTPARFSGSLLGGKAALTLSGVQPEDEAEYYCLLWYSNRWVFGGGTKLTVL |
| 1276 | F7O_LC_V44I | artificial | EVQLVESGGGLVQPGGSLKLSCAASGFTFNVYAMNWVRQAPGKGLEWVARIRSKYNNY |
| | -VH | | ATYYADSVKKRFTISRDDSKNTAYLQMNNLKTEDTAVYYCVRHGNFGNSYISWWAYWG |
| | | | QGTLVTVSS |
| 1277 | F7O_LC_V44I | artificial | QTVVTQEPSLTVSPGGTVTLTCGSSTGAVTSGYYPNWIQQKPGQAPRGLIGGTKFLAP |
| | -VL | | GTPARFSGSLLGGKAALTLSGVQPEDEAEYYCALWYSNRWVFGGGTKLTVL |
| 1278 | F7O_LC_V44L | artificial | EVQLVESGGGLVQPGGSLKLSCAASGFTFNVYAMNWVRQAPGKGLEWVARIRSKYNNY |
| | -VH | | ATYYADSVKKRFTISRDDSKNTAYLQMNNLKTEDTAVYYCVRHGNFGNSYISWWAYWG |
| | | | QGTLVTVSS |
| 1279 | F7O_LC_V44L | artificial | QTVVTQEPSLTVSPGGTVTLTCGSSTGAVTSGYYPNWLQQKPGQAPRGLIGGTKFLAP |
| | -VL | | GTPARFSGSLLGGKAALTLSGVQPEDEAEYYCALWYSNRWVFGGGTKLTVL |
| 1280 | F7O_LC_V44M | artificial | EVQLVESGGGLVQPGGSLKLSCAASGFTFNVYAMNWVRQAPGKGLEWVARIRSKYNNY |
| | -VH | | ATYYADSVKKRFTISRDDSKNTAYLQMNNLKTEDTAVYYCVRHGNFGNSYISWWAYWG |
| | | | QGTLVTVSS |
| 1281 | F7O_LC_V44M | artificial | QTVVTQEPSLTVSPGGTVTLTCGSSTGAVTSGYYPNWMQQKPGQAPRGLIGGTKFLAP |
| | -VL | | GTPARFSGSLLGGKAALTLSGVQPEDEAEYYCALWYSNRWVFGGGTKLTVL |
| 1282 | F7O_LC_W109Y- | artificial | VYAMN |
| | HCDR1 | | |
| 1283 | F7O_LC_W109Y- | artificial | RIRSKYNNYATYYADSVKK |
| | HCDR2 | | |
| 1284 | F7O_LC_W109Y- | artificial | HGNFGNSYISWWAY |
| | HCDR3 | | |
| 1285 | F7O_LC_W109Y- | artificial | GSSTGAVTSGYYPN |
| | LCDR1 | | |
| 1286 | F7O_LC_W109Y- | artificial | GTKFLAP |
| | LCDR2 | | |
| 1287 | F7O_LC_W109Y- | artificial | ALYYSNRWV |
| | LCDR3 | | |
| 1288 | F7O_LC_W109Y- | artificial | EVQLVESGGGLVQPGGSLKLSCAASGFTFNVYAMNWVRQAPGKGLEWVARIRSKYNNY |
| | VH | | ATYYADSVKKRFTISRDDSKNTAYLQMNNLKTEDTAVYYCVRHGNFGNSYISWWAYWG |
| | | | QGTLVTVSS |
| 1289 | F7O_LC_W109Y- | artificial | QTVVTQEPSLTVSPGGTVTLTCGSSTGAVTSGYYPNWVQQKPGQAPRGLIGGTKFLAP |
| | VL | | GTPARFSGSLLGGKAALTLSGVQPEDEAEYYCALYYSNRWVFGGGTKLTVL |
| 1290 | G4H-HCDR1 | artificial | RYAMN |
| 1291 | G4H-HCDR2 | artificial | RIRSKYNNYATYYADSVKG |
| 1292 | G4H-HCDR3 | artificial | HGNFGNSYLSYFAY |
| 1293 | G4H-LCDR1 | artificial | GSSTGAVTSGYYPN |
| 1294 | G4H-LCDR2 | artificial | GTKFLAP |
| 1295 | G4H-LCDR3 | artificial | ALWYSNRWV |
| 1296 | G4H-VH | artificial | EVQLVESGGGLVQPGGSLKLSCAASGFTFNRYAMNWVRQAPGKGLEWVARIRSKYNNY |
| | | | ATYYADSVKGRFTISRDDSKNTAYLQMNNLKTEDTAVYYCVRHGNFGNSYLSYFAYWG |
| | | | QGTLVTVSS |
| 1297 | G4H-VL | artificial | QTVVTQEPSLTVSPGGTVTLTCGSSTGAVTSGYYPNWVQQKPGQAPRGLIGGTKFLAP |
| | | | GTPARFSGSLLGGKAALTLSGVQPEDEAEYYCALWYSNRWVFGGGTKLTVL |
| 1298 | G4H_HC_A89I | artificial | EVQLVESGGGLVQPGGSLKLSCAASGFTFNRYAMNWVRQAPGKGLEWVARIRSKYNNY |
| | -VH | | ATYYADSVKGRFTISRDDSKNTIYLQMNNLKTEDTAVYYCVRHGNFGNSYLSYFAYWG |
| | | | QGTLVTVSS |
| 1299 | G4H_HC_A89I | artificial | QTVVTQEPSLTVSPGGTVTLTCGSSTGAVTSGYYPNWVQQKPGQAPRGLIGGTKFLAP |
| | -VL | | GTPARFSGSLLGGKAALTLSGVQPEDEAEYYCALWYSNRWVFGGGTKLTVL |
| 1300 | G4H_HC_A89L | artificial | EVQLVESGGGLVQPGGSLKLSCAASGFTFNRYAMNWVRQAPGKGLEWVARIRSKYNNY |
| | -VH | | ATYYADSVKGRFTISRDDSKNTLYLQMNNLKTEDTAVYYCVRHGNFGNSYLSYFAYWG |
| | | | QGTLVTVSS |
| 1301 | G4H_HC_A89L | artificial | QTVVTQEPSLTVSPGGTVTLTCGSSTGAVTSGYYPNWVQQKPGQAPRGLIGGTKFLAP |
| | -VL | | GTPARFSGSLLGGKAALTLSGVQPEDEAEYYCALWYSNRWVFGGGTKLTVL |
| 1302 | G4H_HC_A89T | artificial | EVQLVESGGGLVQPGGSLKLSCAASGFTFNRYAMNWVRQAPGKGLEWVARIRSKYNNY |
| | -VH | | ATYYADSVKGRFTISRDDSKNTTYLQMNNLKTEDTAVYYCVRHGNFGNSYLSYFAYWG |
| | | | QGTLVTVSS |
| 1303 | G4H_HC_A89T | artificial | QTVVTQEPSLTVSPGGTVTLTCGSSTGAVTSGYYPNWVQQKPGQAPRGLIGGTKFLAP |
| | -VL | | GTPARFSGSLLGGKAALTLSGVQPEDEAEYYCALWYSNRWVFGGGTKLTVL |
| 1304 | G4H_HC_A89V | artificial | EVQLVESGGGLVQPGGSLKLSCAASGFTFNRYAMNWVRQAPGKGLEWVARIRSKYNNY |
| | -VH | | ATYYADSVKGRFTISRDDSKNTVYLQMNNLKTEDTAVYYCVRHGNFGNSYLSYFAYWG |
| | | | QGTLVTVSS |
| 1305 | G4H_HC_A89V | artificial | QTVVTQEPSLTVSPGGTVTLTCGSSTGAVTSGYYPNWVQQKPGQAPRGLIGGTKFLAP |
| | -VL | | GTPARFSGSLLGGKAALTLSGVQPEDEAEYYCALWYSNRWVFGGGTKLTVL |
| 1306 | G4H_HC_L52M | artificial | EVQLVESGGGLVQPGGSLKLSCAASGFTFNRYAMNWVRQAPGKGMEWVARIRSKYNNY |
| | -VH | | ATYYADSVKGRFTISRDDSKNTAYLQMNNLKTEDTAVYYCVRHGNFGNSYLSYFAYWG |
| | | | QGTLVTVSS |
| 1307 | G4H_HC_L52M | artificial | QTVVTQEPSLTVSPGGTVTLTCGSSTGAVTSGYYPNWVQQKPGQAPRGLIGGTKFLAP |
| | -VL | | GTPARFSGSLLGGKAALTLSGVQPEDEAEYYCALWYSNRWVFGGGTKLTVL |
| 1308 | G4H_HC_L52M_ | artificial | EVQLVESGGGLVQPGGSLKLSCAASGFTFNRYAMNWVRQAPGKGMEWVARIRSKYNNY |
| | LC_L21I_ | | ATYYADSVKGRFTISRDDSKNTAYLQMNNLKTEDTAVYYCVRHGNFGNSYLSYFAYWG |
| | Q46K_G141S- | | QGTLVTVSS |
| | VH | | |
| 1309 | G4H_HC_L52M_ | artificial | QTVVTQEPSLTVSPGGTVTITCGSSTGAVTSGYYPNWVQKKPGQAPRGLIGGTKFLAP |
| | LC_L21I_ | | GTPARFSGSLLGGKAALTLSGVQPEDEAEYYCALWYSNRWVFGSGTKLTVL |
| | Q46K_G141S- | | |
| | VL | | |
| 1310 | G4H_HC_L52M_ | artificial | EVQLVESGGGLVQPGGSLKLSCAASGFTFNRYAMNWVRQAPGKGMEWVARIRSKYNNY |
| | LC_Q46K- | | ATYYADSVKGRFTISRDDSKNTAYLQMNNLKTEDTAVYYCVRHGNFGNSYLSYFAYWG |
| | VH | | QGTLVTVSS |
| 1311 | G4H_HC_L52M_ | artificial | QTVVTQEPSLTVSPGGTVTLTCGSSTGAVTSGYYPNWVQKKPGQAPRGLIGGTKFLAP |
| | LC_Q46K- | | GTPARFSGSLLGGKAALTLSGVQPEDEAEYYCALWYSNRWVFGGGTKLTVL |
| | VL | | |
| 1312 | G4H_HC_L52M_ | artificial | EVQLVESGGGLVQPGGSLKLSCAASGFTFNRYAMNWVRQAPGKGMEWVARIRSKYNNY |
| | LC_Q46K_ | | ATYYADSVKGRFTISRDDSKNTAYLQMNNLKTEDTAVYYCVRHGNFGNSYLSYFAYWG |
| | G141S-VH | | QGTLVTVSS |
| 1313 | G4H_HC_L52M_ | artificial | QTVVTQEPSLTVSPGGTVTLTCGSSTGAVTSGYYPNWVQKKPGQAPRGLIGGTKFLAP |
| | LC_Q46K_ | | GTPARFSGSLLGGKAALTLSGVQPEDEAEYYCALWYSNRWVFGSGTKLTVL |
| | G141S-VL | | |
| 1314 | G4H_HC_L52V | artificial | EVQLVESGGGLVQPGGSLKLSCAASGFTFNRYAMNWVRQAPGKGVEWVARIRSKYNNY |
| | -VH | | ATYYADSVKGRFTISRDDSKNTAYLQMNNLKTEDTAVYYCVRHGNFGNSYLSYFAYWG |
| | | | QGTLVTVSS |
| 1315 | G4H_HC_L52V | artificial | QTVVTQEPSLTVSPGGTVTLTCGSSTGAVTSGYYPNWVQQKPGQAPRGLIGGTKFLAP |
| | -VL | | GTPARFSGSLLGGKAALTLSGVQPEDEAEYYCALWYSNRWVFGGGTKLTVL |
| 1316 | G4H_HC_L52V | artificial | EVQLVESGGGLVQPGGSLKLSCAASGFTFNRYAMNWVRQAPGKGVEWVARIRSKYNNY |
| | LC_V44F- | | ATYYADSVKGRFTISRDDSKNTAYLQMNNLKTEDTAVYYCVRHGNFGNSYLSYFAYWG |
| | VH | | QGTLVTVSS |
| 1317 | G4H_HC_L52V_ | artificial | QTVVTQEPSLTVSPGGTVTLTCGSSTGAVTSGYYPNWFQQKPGQAPRGLIGGTKFLAP |
| | LC_V44F- | | GTPARFSGSLLGGKAALTLSGVQPEDEAEYYCALWYSNRWVFGGGTKLTVL |
| | VL | | |
| 1318 | G4H_HC_L52V_ | artificial | EVQLVESGGGLVQPGGSLKLSCAASGFTFNRYAMNWVRQAPGKGVEWVARIRSKYNNY |
| | LC_V44Y- | | ATYYADSVKGRFTISRDDSKNTAYLQMNNLKTEDTAVYYCVRHGNFGNSYLSYFAYWG |
| | VH | | QGTLVTVSS |
| 1319 | G4H_HC_L52V_ | artificial | QTVVTQEPSLTVSPGGTVTLTCGSSTGAVTSGYYPNWYQQKPGQAPRGLIGGTKFLAP |
| | LC_V44Y- | | GTPARFSGSLLGGKAALTLSGVQPEDEAEYYCALWYSNRWVFGGGTKLTVL |
| | VL | | |
| 1320 | G4H_HC_M41F_ | artificial | RYAFN |
| | A89V- | | |
| | HCDR1 | | |
| 1321 | G4H_HC_M41F_ | artificial | RIRSKYNNYATYYADSVKG |
| | A89V- | | |
| | HCDR2 | | |
| 1322 | G4H_HC_M41F_ | artificial | HGNFGNSYLSYFAY |
| | A89V-HCDR3 | | |
| 1323 | G4H_HC_M41F_ | artificial | GSSTGAVTSGYYPN |
| | A89V-LCDR1 | | |
| 1324 | G4H_HC_M41F_ | artificial | GTKFLAP |
| | A89V-LCDR2 | | |
| 1325 | G4H_HC_M41F_ | artificial | ALWYSNRWV |
| | A89V-LCDR3 | | |
| 1326 | G4H_HC_M41F_ | artificial | EVQLVESGGGLVQPGGSLKLSCAASGFTFNRYAFNWVRQAPGKGLEWVARIRSKYNNY |
| | A89V-VH | | ATYYADSVKGRFTISRDDSKNTVYLQMNNLKTEDTAVYYCVRHGNFGNSYLSYFAYWG |
| | | | QGTLVTVSS |
| 1327 | G4H_HC_M41F_ | artificial | QTVVTQEPSLTVSPGGTVTLTCGSSTGAVTSGYYPNWVQQKPGQAPRGLIGGTKFLAP |
| | A89V-VL | | GTPARFSGSLLGGKAALTLSGVQPEDEAEYYCALWYSNRWVFGGGTKLTVL |
| 1328 | G4H_HC_M41I | artificial | RYAIN |
| | -HCDR1 | | |
| 1329 | G4H_HC_M41I | artificial | RIRSKYNNYATYYADSVKG |
| | -HCDR2 | | |
| 1330 | G4H_HC_M41I | artificial | HGNFGNSYLSYFAY |
| | -HCDR3 | | |
| 1331 | G4H_HC_M41I | artificial | GSSTGAVTSGYYPN |
| | -LCDR1 | | |
| 1332 | G4H_HC_M41I | artificial | GTKFLAP |
| | -LCDR2 | | |
| 1333 | G4H_HC_M41I | artificial | ALWYSNRWV |
| | -LCDR3 | | |
| 1334 | G4H_HC_M41I | artificial | EVQLVESGGGLVQPGGSLKLSCAASGFTFNRYAINWVRQAPGKGLEWVARIRSKYNNY |
| | -VH | | ATYYADSVKGRFTISRDDSKNTAYLQMNNLKTEDTAVYYCVRHGNFGNSYLSYFAYWG |
| | | | QGTLVTVSS |
| 1335 | G4H_HC_M41I | artificial | QTVVTQEPSLTVSPGGTVTLTCGSSTGAVTSGYYPNWVQQKPGQAPRGLIGGTKFLAP |
| | -VL | | GTPARFSGSLLGGKAALTLSGVQPEDEAEYYCALWYSNRWVFGGGTKLTVL |
| 1336 | G4H_HC_M41I_ | artificial | EVQLVESGGGLVQPGGSLKLSCAASGFTFNRYAINWVRQAPGKGLEWVARIRSKYNNY |
| | A89L-VH | | ATYYADSVKGRFTISRDDSKNTLYLQMNNLKTEDTAVYYCVRHGNFGNSYLSYFAYWG |
| | | | QGTLVTVSS |
| 1337 | G4H_HC_M41I_ | artificial | QTVVTQEPSLTVSPGGTVTLTCGSSTGAVTSGYYPNWVQQKPGQAPRGLIGGTKFLAP |
| | A89L-VL | | GTPARFSGSLLGGKAALTLSGVQPEDEAEYYCALWYSNRWVFGGGTKLTVL |
| 1338 | G4H_HC_M41I_ | artificial | EVQLVESGGGLVQPGGSLKLSCAASGFTFNRYAINWVRQAPGKGLEWVARIRSKYNNY |
| | A89V-VH | | ATYYADSVKGRFTISRDDSKNTVYLQMNNLKTEDTAVYYCVRHGNFGNSYLSYFAYWG |
| | | | QGTLVTVSS |
| 1339 | G4H_HC_M41I_ | artificial | QTVVTQEPSLTVSPGGTVTLTCGSSTGAVTSGYYPNWVQQKPGQAPRGLIGGTKFLAP |
| | A89V-VL | | GTPARFSGSLLGGKAALTLSGVQPEDEAEYYCALWYSNRWVFGGGTKLTVL |
| 1340 | G4H_HC_M41I_ | artificial | EVQLVESGGGLVQPGGSLKLSCAASGFTFNRYAINWVRQAPGKGLEWVARIRSKYNNY |
| | A89V_LC_ | | ATYYADSVKGRFTISRDDSKNTVYLQMNNLKTEDTAVYYCVRHGNFGNSYLSYFAYWG |
| | L21I_Q46K_ | | QGTLVTVSS |
| | G141S-VH | | |
| 1341 | G4H_HC_M41I_ | artificial | QTVVTQEPSLTVSPGGTVTITCGSSTGAVTSGYYPNWVQKKPGQAPRGLIGGTKFLAP |
| | A89V_LC_ | | GTPARFSGSLLGGKAALTLSGVQPEDEAEYYCALWYSNRWVFGSGTKLTVL |
| | L21I_Q46K_ | | |
| | 1G14S-VL | | |
| 1342 | G4H_HC_M41I_ | artificial | EVQLVESGGGLVQPGGSLKLSCAASGFTFNRYAINWVRQAPGKGLEWVARIRSKYNNY |
| | A89V_LC_ | | ATYYADSVKGRFTISRDDSKNTVYLQMNNLKTEDTAVYYCVRHGNFGNSYLSYFAYWG |
| | Q46K-VH | | QGTLVTVSS |
| 1343 | G4H_HC_M41I_ | artificial | QTVVTQEPSLTVSPGGTVTLTCGSSTGAVTSGYYPNWVQKKPGQAPRGLIGGTKFLAP |
| | A89V_LC_ | | GTPARFSGSLLGGKAALTLSGVQPEDEAEYYCALWYSNRWVFGGGTKLTVL |
| | Q46K-VL | | |
| 1344 | G4H_HC_M41I_ | artificial | EVQLVESGGGLVQPGGSLKLSCAASGFTFNRYAINWVRQAPGKGVEWVARIRSKYNNY |
| | L52V_A89V_ | | ATYYADSVKGRFTISRDDSKNTVYLQMNNLKTEDTAVYYCVRHGNFGNSYLSYFAYWG |
| | LC_L21I_ | | QGTLVTVSS |
| | Q46K_G141S_ | | |
| | V44F-VH | | |
| 1345 | G4H_HC_M41I_ | artificial | QTVVTQEPSLTVSPGGTVTITCGSSTGAVTSGYYPNWFQKKPGQAPRGLIGGTKFLAP |
| | L52V_A89V_ | | GTPARFSGSLLGGKAALTLSGVQPEDEAEYYCALWYSNRWVFGSGTKLTVL |
| | LC_L21I_ | | |
| | Q46K_G141S_ | | |
| | V44F-VL | | |
| 1346 | G4H_HC_M41I_ | artificial | EVQLVESGGGLVQPGGSLKLSCAASGFTFNRYAINWVRQAPGKGLEWVARIRSKYNNY |
| | LC_L21I_ | | ATYYADSVKGRFTISRDDSKNTAYLQMNNLKTEDTAVYYCVRHGNFGNSYLSYFAYWG |
| | Q46K_G141S- | | QGTLVTVSS |
| | VH | | |
| 1347 | G4H_HC_M41I_ | artificial | QTVVTQEPSLTVSPGGTVTITCGSSTGAVTSGYYPNWVQKKPGQAPRGLIGGTKFLAP |
| | LC_L21I_ | | GTPARFSGSLLGGKAALTLSGVQPEDEAEYYCALWYSNRWVFGSGTKLTVL |
| | Q46K_G141S- | | |
| | VL | | |
| 1348 | G4H_HC_M41I_ | artificial | EVQLVESGGGLVQPGGSLKLSCAASGFTFNRYAINWVREAPGKGLEWVARIRSKYNNY |
| | Q46E_A89V_ | | ATYYADSVKGRFTISRDDSKNTVYLQMNNLKTEDTAVYYCVRHGNFGNSYLSYFAYWG |
| | LC_L21I_ | | QGTLVTVSS |
| | Q46K_G141S- | | |
| | VH | | |
| 1349 | G4H_HC_M41I_ | artificial | QTVVTQEPSLTVSPGGTVTITCGSSTGAVTSGYYPNWVQKKPGQAPRGLIGGTKFLAP |
| | Q46E_A89V_ | | GTPARFSGSLLGGKAALTLSGVQPEDEAEYYCALWYSNRWVFGSGTKLTVL |
| | LC_L21I_ | | |
| | Q46K_G141S- | | |
| | VL | | |
| 1350 | G4H_HC_M41V | artificial | RYAVN |
| | -HCDR1 | | |
| 1351 | G4H_HC_M41V | artificial | RIRSKYNNYATYYADSVKG |
| | -HCDR2 | | |
| 1352 | G4H_HC_M41V | artificial | HGNFGNSYLSYFAY |
| | -HCDR3 | | |
| 1353 | G4H_HC_M41V | artificial | GSSTGAVTSGYYPN |
| | -LCDR1 | | |
| 1354 | G4H_HC_M41V | artificial | GTKFLAP |
| | -LCDR2 | | |
| 1355 | G4H_HC_M41V | artificial | ALWYSNRWV |
| | -LCDR3 | | |
| 1356 | G4H_HC_M41V | artificial | EVQLVESGGGLVQPGGSLKLSCAASGFTFNRYAVNWVRQAPGKGLEWVARIRSKYNNY |
| | -VH | | ATYYADSVKGRFTISRDDSKNTAYLQMNNLKTEDTAVYYCVRHGNFGNSYLSYFAYWG |
| | | | QGTLVTVSS |
| 1357 | G4H_HC_M41V | artificial | QTVVTQEPSLTVSPGGTVTLTCGSSTGAVTSGYYPNWVQQKPGQAPRGLIGGTKFLAP |
| | -VL | | GTPARFSGSLLGGKAALTLSGVQPEDEAEYYCALWYSNRWVFGGGTKLTVL |
| 1358 | G4H_HC_Q46D | artificial | EVQLVESGGGLVQPGGSLKLSCAASGFTFNRYAMNWVRDAPGKGLEWVARIRSKYNNY |
| | -VH | | ATYYADSVKGRFTISRDDSKNTAYLQMNNLKTEDTAVYYCVRHGNFGNSYLSYFAYWG |
| | | | QGTLVTVSS |
| 1359 | G4H_HC_Q46D | artificial | QTVVTQEPSLTVSPGGTVTLTCGSSTGAVTSGYYPNWVQQKPGQAPRGLIGGTKFLAP |
| | -VL | | GTPARFSGSLLGGKAALTLSGVQPEDEAEYYCALWYSNRWVFGGGTKLTVL |
| 1360 | G4H_HC_Q46D_ | artificial | EVQLVESGGGLVQPGGSLKLSCAASGFTFNRYAMNWVRDAPGKGLEWVARIRSKYNNY |
| | LC_L21I_ | | ATYYADSVKGRFTISRDDSKNTAYLQMNNLKTEDTAVYYCVRHGNFGNSYLSYFAYWG |
| | Q46K_G141S- | | QGTLVTVSS |
| | VH | | |
| 1361 | G4H_HC_Q46D_ | artificial | QTVVTQEPSLTVSPGGTVTITCGSSTGAVTSGYYPNWVQKKPGQAPRGLIGGTKFLAP |
| | LC_L21I_ | | GTPARFSGSLLGGKAALTLSGVQPEDEAEYYCALWYSNRWVFGSGTKLTVL |
| | Q46K_G141S- | | |
| | VL | | |
| 1362 | G4H_HC_Q46D_ | artificial | EVQLVESGGGLVQPGGSLKLSCAASGFTFNRYAMNWVRDAPGKGLEWVARIRSKYNNY |
| | LC_L21I_ | | ATYYADSVKGRFTISRDDSKNTAYLQMNNLKTEDTAVYYCVRHGNFGNSYLSYFAYWG |
| | Q46R_G141S- | | QGTLVTVSS |
| | VH | | |
| 1363 | G4H_HC_Q46D_ | artificial | QTVVTQEPSLTVSPGGTVTITCGSSTGAVTSGYYPNWVQRKPGQAPRGLIGGTKFLAP |
| | LC_L21I_ | | GTPARFSGSLLGGKAALTLSGVQPEDEAEYYCALWYSNRWVFGSGTKLTVL |
| | Q46R_G141S- | | |
| | VL | | |
| 1364 | G4H_HC_Q46D_ | artificial | EVQLVESGGGLVQPGGSLKLSCAASGFTFNRYAMNWVRDAPGKGLEWVARIRSKYNNY |
| | LC_Q46K- | | ATYYADSVKGRFTISRDDSKNTAYLQMNNLKTEDTAVYYCVRHGNFGNSYLSYFAYWG |
| | VH | | QGTLVTVSS |
| 1365 | G4H_HC_Q46D_ | artificial | QTVVTQEPSLTVSPGGTVTLTCGSSTGAVTSGYYPNWVQKKPGQAPRGLIGGTKFLAP |
| | LC_Q46K- | | GTPARFSGSLLGGKAALTLSGVQPEDEAEYYCALWYSNRWVFGGGTKLTVL |
| | VL | | |
| 1366 | G4H_HC_Q46D_ | artificial | EVQLVESGGGLVQPGGSLKLSCAASGFTFNRYAMNWVRDAPGKGLEWVARIRSKYNNY |
| | LC_Q46K_ | | ATYYADSVKGRFTISRDDSKNTAYLQMNNLKTEDTAVYYCVRHGNFGNSYLSYFAYWG |
| | G141S-VH | | QGTLVTVSS |
| 1367 | G4H_HC_Q46D_ | artificial | QTVVTQEPSLTVSPGGTVTLTCGSSTGAVTSGYYPNWVQKKPGQAPRGLIGGTKFLAP |
| | LC_Q46K_ | | GTPARFSGSLLGGKAALTLSGVQPEDEAEYYCALWYSNRWVFGSGTKLTVL |
| | G141S-VL | | |
| 1368 | G4H_HC_Q46D_ | artificial | EVQLVESGGGLVQPGGSLKLSCAASGFTFNRYAMNWVRDAPGKGLEWVARIRSKYNNY |
| | LC_Q46R- | | ATYYADSVKGRFTISRDDSKNTAYLQMNNLKTEDTAVYYCVRHGNFGNSYLSYFAYWG |
| | VH | | QGTLVTVSS |
| 1369 | G4H_HC_Q46D_ | artificial | QTVVTQEPSLTVSPGGTVTLTCGSSTGAVTSGYYPNWVQRKPGQAPRGLIGGTKFLAP |
| | LC_Q46R- | | GTPARFSGSLLGGKAALTLSGVQPEDEAEYYCALWYSNRWVFGGGTKLTVL |
| | VL | | |
| 1370 | G4H_HC_Q46D_ | artificial | EVQLVESGGGLVQPGGSLKLSCAASGFTFNRYAMNWVRDAPGKGLEWVARIRSKYNNY |
| | LC_Q46R_ | | ATYYADSVKGRFTISRDDSKNTAYLQMNNLKTEDTAVYYCVRHGNFGNSYLSYFAYWG |
| | G141S-VH | | QGTLVTVSS |
| 1371 | G4H_HC_Q46D_ | artificial | QTVVTQEPSLTVSPGGTVTLTCGSSTGAVTSGYYPNWVQRKPGQAPRGLIGGTKFLAP |
| | LC_Q46R_ | | GTPARFSGSLLGGKAALTLSGVQPEDEAEYYCALWYSNRWVFGSGTKLTVL |
| | G141S-VL | | |
| 1372 | G4H_HC_Q46E | artificial | EVQLVESGGGLVQPGGSLKLSCAASGFTFNRYAMNWVREAPGKGLEWVARIRSKYNNY |
| | -VH | | ATYYADSVKGRFTISRDDSKNTAYLQMNNLKTEDTAVYYCVRHGNFGNSYLSYFAYWG |
| | | | QGTLVTVSS |
| 1373 | G4H_HC_Q46E | artificial | QTVVTQEPSLTVSPGGTVTLTCGSSTGAVTSGYYPNWVQQKPGQAPRGLIGGTKFLAP |
| | -VL | | GTPARFSGSLLGGKAALTLSGVQPEDEAEYYCALWYSNRWVFGGGTKLTVL |
| 1374 | G4H_HC_Q46E_ | artificial | EVQLVESGGGLVQPGGSLKLSCAASGFTFNRYAMNWVREAPGKGLEWVARIRSKYNNY |
| | LC_L21I_ | | ATYYADSVKGRFTISRDDSKNTAYLQMNNLKTEDTAVYYCVRHGNFGNSYLSYFAYWG |
| | Q46K_G141S- | | QGTLVTVSS |
| | VH | | |
| 1375 | G4H_HC_Q46E_ | artificial | QTVVTQEPSLTVSPGGTVTITCGSSTGAVTSGYYPNWVQKKPGQAPRGLIGGTKFLAP |
| | LC_L21I_ | | GTPARFSGSLLGGKAALTLSGVQPEDEAEYYCALWYSNRWVFGSGTKLTVL |
| | Q46K_G141S- | | |
| | VL | | |
| 1376 | G4H_HC_Q46E_ | artificial | EVQLVESGGGLVQPGGSLKLSCAASGFTFNRYAMNWVREAPGKGLEWVARIRSKYNNY |
| | LC_L21I_ | | ATYYADSVKGRFTISRDDSKNTAYLQMNNLKTEDTAVYYCVRHGNFGNSYLSYFAYWG |
| | 6Q4R_G141S- | | QGTLVTVSS |
| | VH | | |
| 1377 | G4H_HC_Q46E_ | artificial | QTVVTQEPSLTVSPGGTVTITCGSSTGAVTSGYYPNWVQRKPGQAPRGLIGGTKFLAP |
| | LC_L21I_ | | GTPARFSGSLLGGKAALTLSGVQPEDEAEYYCALWYSNRWVFGSGTKLTVL |
| | Q46R_G141S- | | |
| | VL | | |
| 1378 | G4H_HC_Q46E_ | artificial | EVQLVESGGGLVQPGGSLKLSCAASGFTFNRYAMNWVREAPGKGLEWVARIRSKYNNY |
| | LC_Q46K- | | ATYYADSVKGRFTISRDDSKNTAYLQMNNLKTEDTAVYYCVRHGNFGNSYLSYFAYWG |
| | VH | | QGTLVTVSS |
| 1379 | G4H_HC_Q46E_ | artificial | QTVVTQEPSLTVSPGGTVTLTCGSSTGAVTSGYYPNWVQKKPGQAPRGLIGGTKFLAP |
| | LC_Q46K- | | GTPARFSGSLLGGKAALTLSGVQPEDEAEYYCALWYSNRWVFGGGTKLTVL |
| | VL | | |
| 1380 | G4H_HC_Q46E_ | artificial | EVQLVESGGGLVQPGGSLKLSCAASGFTFNRYAMNWVREAPGKGLEWVARIRSKYNNY |
| | LC_Q46K_ | | ATYYADSVKGRFTISRDDSKNTAYLQMNNLKTEDTAVYYCVRHGNFGNSYLSYFAYWG |
| | 4G11S-VH | | QGTLVTVSS |
| 1381 | G4H_HC_Q46E_ | artificial | QTVVTQEPSLTVSPGGTVTLTCGSSTGAVTSGYYPNWVQKKPGQAPRGLIGGTKFLAP |
| | LC_Q46K_ | | GTPARFSGSLLGGKAALTLSGVQPEDEAEYYCALWYSNRWVFGSGTKLTVL |
| | G141S-VL | | |
| 1382 | G4H_HC_Q46E_ | artificial | EVQLVESGGGLVQPGGSLKLSCAASGFTFNRYAMNWVREAPGKGLEWVARIRSKYNNY |
| | LC_Q46R- | | ATYYADSVKGRFTISRDDSKNTAYLQMNNLKTEDTAVYYCVRHGNFGNSYLSYFAYWG |
| | VH | | QGTLVTVSS |
| 1383 | G4H_HC_Q46E_ | artificial | QTVVTQEPSLTVSPGGTVTLTCGSSTGAVTSGYYPNWVQRKPGQAPRGLIGGTKFLAP |
| | LC_Q46R- | | GTPARFSGSLLGGKAALTLSGVQPEDEAEYYCALWYSNRWVFGGGTKLTVL |
| | VL | | |
| 1384 | G4H_HC_Q46E_ | artificial | EVQLVESGGGLVQPGGSLKLSCAASGFTFNRYAMNWVREAPGKGLEWVARIRSKYNNY |
| | LC_Q46R_ | | ATYYADSVKGRFTISRDDSKNTAYLQMNNLKTEDTAVYYCVRHGNFGNSYLSYFAYWG |
| | G141S-VH | | QGTLVTVSS |
| 1385 | G4H_HC_Q46E_ | artificial | QTVVTQEPSLTVSPGGTVTLTCGSSTGAVTSGYYPNWVQRKPGQAPRGLIGGTKFLAP |
| | LC_Q46R_ | | GTPARFSGSLLGGKAALTLSGVQPEDEAEYYCALWYSNRWVFGSGTKLTVL |
| | G141S-VL | | |
| 1386 | G4H_HC_Q46K | artificial | EVQLVESGGGLVQPGGSLKLSCAASGFTFNRYAMNWVRKAPGKGLEWVARIRSKYNNY |
| | -VH | | ATYYADSVKGRFTISRDDSKNTAYLQMNNLKTEDTAVYYCVRHGNFGNSYLSYFAYWG |
| | | | QGTLVTVSS |
| 1387 | G4H_HC_Q46K | artificial | QTVVTQEPSLTVSPGGTVTLTCGSSTGAVTSGYYPNWVQQKPGQAPRGLIGGTKFLAP |
| | -VL | | GTPARFSGSLLGGKAALTLSGVQPEDEAEYYCALWYSNRWVFGGGTKLTVL |
| 1388 | G4H_HC_Q46R | artificial | EVQLVESGGGLVQPGGSLKLSCAASGFTFNRYAMNWVRRAPGKGLEWVARIRSKYNNY |
| | -VH | | ATYYADSVKGRFTISRDDSKNTAYLQMNNLKTEDTAVYYCVRHGNFGNSYLSYFAYWG |
| | | | QGTLVTVSS |
| 1389 | G4H_HC_Q46R | artificial | QTVVTQEPSLTVSPGGTVTLTCGSSTGAVTSGYYPNWVQQKPGQAPRGLIGGTKFLAP |
| | -VL | | GTPARFSGSLLGGKAALTLSGVQPEDEAEYYCALWYSNRWVFGGGTKLTVL |
| 1390 | G4H_HC_V107 | artificial | EVQLVESGGGLVQPGGSLKLSCAASGFTFNRYAMNWVRQAPGKGLEWVARIRSKYNNY |
| | A-VH | | ATYYADSVKGRFTISRDDSKNTAYLQMNNLKTEDTAVYYCARHGNFGNSYLSYFAYWG |
| | | | QGTLVTVSS |
| 1391 | G4H_HC_V107A- | artificial | QTVVTQEPSLTVSPGGTVTLTCGSSTGAVTSGYYPNWVQQKPGQAPRGLIGGTKFLAP |
| | VL | | GTPARFSGSLLGGKAALTLSGVQPEDEAEYYCALWYSNRWVFGGGTKLTVL |
| 1392 | G4H_HC_V107L- | artificial | EVQLVESGGGLVQPGGSLKLSCAASGFTFNRYAMNWVRQAPGKGLEWVARIRSKYNNY |
| | VH | | ATYYADSVKGRFTISRDDSKNTAYLQMNNLKTEDTAVYYCLRHGNFGNSYLSYFAYWG |
| | | | QGTLVTVSS |
| 1393 | G4H_HC_V107L- | artificial | QTVVTQEPSLTVSPGGTVTLTCGSSTGAVTSGYYPNWVQQKPGQAPRGLIGGTKFLAP |
| | VL | | GTPARFSGSLLGGKAALTLSGVQPEDEAEYYCALWYSNRWVFGGGTKLTVL |
| 1394 | G4H_I2E- | artificial | RYAIN |
| | HCDR1 | | |
| 1395 | G4H_I2E- | artificial | RIRSKYNNYATYYADAVKG |
| | HCDR2 | | |
| 1396 | G4H_I2E- | artificial | AGNFGSSYLSYFAY |
| | HCDR3 | | |
| 1397 | G4H_I2E- | artificial | GSSTGAVTSGYYPN |
| | LCDR1 | | |
| 1398 | G4H_I2E- | artificial | GTKFLAP |
| | LCDR2 | | |
| 1399 | G4H_I2E- | artificial | ALWYSNRWV |
| | LCDR3 | | |
| 1400 | G4H_I2E- | artificial | EVQLVESGGGLVQPGGSLKLSCAASGFTFNRYAINWVRQAPGKGLEWVARIRSKYNNY |
| | VH | | ATYYADAVKGRFTISRDDSKNTVYLQMNNLKTEDTAVYYCARAGNFGSSYLSYFAYWG |
| | | | QGTLVTVSS |
| 1401 | G4H_I2E- | artificial | QTVVTQEPSLTVSPGGTVTITCGSSTGAVTSGYYPNWVQKKPGQAPRGLIGGTKFLAP |
| | VL | | GTPARFSGSLSGGKAALTLSGVQPEDEAEYYCALWYSNRWVFGSGTKLTVL |
| 1402 | G4H_I2K- | artificial | RYAMN |
| | HCDR1 | | |
| 1403 | G4H_I2K- | artificial | RIRSKYNNYATYYAEAVKG |
| | HCDR2 | | |
| 1404 | G4H_I2K- | artificial | NENIGTSYLSYFAY |
| | HCDR3 | | |
| 1405 | G4H_I2K- | artificial | GSSTGAVTSGYYPN |
| | LCDR1 | | |
| 1406 | G4H_I2K- | artificial | GTKFLAP |
| | LCDR2 | | |
| 1407 | G4H_I2K- | artificial | ALWYSNRWV |
| | LCDR3 | | |
| 1408 | G4H_I2K- | artificial | EVQLVESGGGLVQPGGSLKLSCAASGFTFNRYAMNWVRQAPGKGLEWVARIRSKYNNY |
| | VH | | ATYYAEAVKGRFTISRDDSKNTVYLQMNNLKTEDTAVYYCVRNENIGTSYLSYFAYWG |
| | | | QGTLVTVSS |
| 1409 | G4H_I2K- | artificial | QTVVTQEPSLTVSPGGTVTITCGSSTGAVTSGYYPNWVQQKPGQAPRGLIGGTKFLAP |
| | VL | | GTPARFSGSLSGGKAALTLSGVQPEDEAEYYCALWYSNRWVFGSGTKLTVL |
| 1410 | G4H_I2L- | artificial | RYAMN |
| | HCDR1 | | |
| 1411 | G4H_I2L- | artificial | RIRSKYNNYATYYADAVKG |
| | HCDR2 | | |
| 1412 | G4H_I2L- | artificial | AGNFGSSYLSYFAY |
| | HCDR3 | | |
| 1413 | G4H_I2L- | artificial | GSSTGAVTSGYYPN |
| | LCDR1 | | |
| 1414 | G4H_I2L- | artificial | GTKFLAP |
| | LCDR2 | | |
| 1415 | G4H_I2L- | artificial | ALYYSNRWV |
| | LCDR3 | | |
| 1416 | G4H_I2L- | artificial | EVQLVESGGGLVQPGGSLKLSCAASGFTFNRYAMNWVRQAPGKGMEWVARIRSKYNNY |
| | VH | | ATYYADAVKGRFTISRDDSKNTLYLQMNNLKTEDTAVYYCVRAGNFGSSYLSYFAYWG |
| | | | QGTLVTVSS |
| 1417 | G4H_I2L- | artificial | QTVVTQEPSLTVSPGGTVTITCGSSTGAVTSGYYPNWIQKKPGQAPRGLIGGTKFLAP |
| | VL | | GTPARFSGSLEGGKAALTLSGVQPEDEAEYYCALYYSNRWVFGSGTKLTVL |
| 1418 | G4H_I2M- | artificial | RYAMN |
| | HCDR1 | | |
| 1419 | G4H_I2M- | artificial | RIRSKYNNYATYYADAVKG |
| | HCDR2 | | |
| 1420 | G4H_I2M- | artificial | AGNFGTSYLSYFAY |
| | HCDR3 | | |
| 1421 | G4H_I2M- | artificial | GSSTGAVTSGYYPN |
| | LCDR1 | | |
| 1422 | G4H_I2M- | artificial | GTKFLAP |
| | LCDR2 | | |
| 1423 | G4H_I2M- | artificial | ALWYSNRWV |
| | LCDR3 | | |
| 1424 | G4H_I2M | artificial | EVQLVESGGGLVQPGGSLKLSCAASGFTFNRYAMNWVRQAPGKGMEWVARIRSKYNNY |
| | VH | | ATYYADAVKGRFTISRDDSKNTLYLQMNNLKTEDTAVYYCVRAGNFGTSYLSYFAYWG |
| | | | QGTLVTVSS |
| 1425 | G4H_I2M- | artificial | QTVVTQEPSLTVSPGGTVTITCGSSTGAVTSGYYPNWVQKKPGQAPRGLIGGTKFLAP |
| | VL | | GTPARFSGSLSGGKAALTLSGVQPEDEAEYYCALWYSNRWVFGSGTKLTVL |
| 1426 | G4H_I2M2- | artificial | RYAIN |
| | HCDR1 | | |
| 1427 | G4H_I2M2- | artificial | RIRSKYNNYATYYADAVKG |
| | HCDR2 | | |
| 1428 | G4H_I2M2- | artificial | NANFGTSYLSYFAY |
| | HCDR3 | | |
| 1429 | G4H_I2M2- | artificial | GSSTGAVTSGYYPN |
| | LCDR1 | | |
| 1430 | G4H_I2M2- | artificial | GTKFLAP |
| | LCDR2 | | |
| 1431 | G4H_I2M2- | artificial | ALWYSNRWV |
| | LCDR3 | | |
| 1432 | G4H_I2M2- | artificial | EVQLVESGGGLVQPGGSLKLSCAASGFTFNRYAINWVREAPGKGLEWVARIRSKYNNY |
| | VH | | ATYYADAVKGRFTISRDDSKNTAYLQMNNLKTEDTAVYYCVRNANFGTSYLSYFAYWG |
| | | | QGTLVTVSS |
| 1433 | G4H_I2M2 | artificial | QTVVTQEPSLTVSPGGTVTLTCGSSTGAVTSGYYPNWVQKKPGQAPRGLIGGTKFLAP |
| | VL | | GTPARFSGSLLGGKAALTLSGVQPEDEAEYYCALWYSNRWVFGSGTKLTVL |
| 1434 | G4H_LC_G141S- | artificial | EVQLVESGGGLVQPGGSLKLSCAASGFTFNRYAMNWVRQAPGKGLEWVARIRSKYNNY |
| | VH | | ATYYADSVKGRFTISRDDSKNTAYLQMNNLKTEDTAVYYCVRHGNFGNSYLSYFAYWG |
| | | | QGTLVTVSS |
| 1435 | G4H_LC_G141S- | artificial | QTVVTQEPSLTVSPGGTVTLTCGSSTGAVTSGYYPNWVQQKPGQAPRGLIGGTKFLAP |
| | VL | | GTPARFSGSLLGGKAALTLSGVQPEDEAEYYCALWYSNRWVFGSGTKLTVL |
| 1436 | G4H_LC_G141S_ | artificial | EVQLVESGGGLVQPGGSLKLSCAASGFTFNRYAMNWVRQAPGKGLEWVARIRSKYNNY |
| | L21I-VH | | ATYYADSVKGRFTISRDDSKNTAYLQMNNLKTEDTAVYYCVRHGNFGNSYLSYFAYWG |
| | | | QGTLVTVSS |
| 1437 | G4H_LC_G141S_ | artificial | QTVVTQEPSLTVSPGGTVTITCGSSTGAVTSGYYPNWVQQKPGQAPRGLIGGTKFLAP |
| | L21I-VL | | GTPARFSGSLLGGKAALTLSGVQPEDEAEYYCALWYSNRWVFGSGTKLTVL |
| 1438 | G4H_LC_L83E | artificial | EVQLVESGGGLVQPGGSLKLSCAASGFTFNRYAMNWVRQAPGKGLEWVARIRSKYNNY |
| | -VH | | ATYYADSVKGRFTISRDDSKNTAYLQMNNLKTEDTAVYYCVRHGNFGNSYLSYFAYWG |
| | | | QGTLVTVSS |
| 1439 | G4H_LC_L83E | artificial | QTVVTQEPSLTVSPGGTVTLTCGSSTGAVTSGYYPNWVQQKPGQAPRGLIGGTKFLAP |
| | -VL | | GTPARFSGSLEGGKAALTLSGVQPEDEAEYYCALWYSNRWVFGGGTKLTVL |
| 1440 | G4H_LC_L83S | artificial | EVQLVESGGGLVQPGGSLKLSCAASGFTFNRYAMNWVRQAPGKGLEWVARIRSKYNNY |
| | -VH | | ATYYADSVKGRFTISRDDSKNTAYLQMNNLKTEDTAVYYCVRHGNFGNSYLSYFAYWG |
| | | | QGTLVTVSS |
| 1441 | G4H_LC_L83S | artificial | QTVVTQEPSLTVSPGGTVTLTCGSSTGAVTSGYYPNWVQQKPGQAPRGLIGGTKFLAP |
| | -VL | | GTPARFSGSLSGGKAALTLSGVQPEDEAEYYCALWYSNRWVFGGGTKLTVL |
| 1442 | G4H_LC_Q46E | artificial | EVQLVESGGGLVQPGGSLKLSCAASGFTENRYAMNWVRQAPGKGLEWVARIRSKYNNY |
| | -VH | | ATYYADSVKGRFTISRDDSKNTAYLQMNNLKTEDTAVYYCVRHGNFGNSYLSYFAYWG |
| | | | QGTLVTVSS |
| 1443 | G4H_LC_Q46E | artificial | QTVVTQEPSLTVSPGGTVTLTCGSSTGAVTSGYYPNWVQEKPGQAPRGLIGGTKFLAP |
| | -VL | | GTPARFSGSLLGGKAALTLSGVQPEDEAEYYCALWYSNRWVFGGGTKLTVL |
| 1444 | G4H_LC_Q46K | artificial | EVQLVESGGGLVQPGGSLKLSCAASGFTFNRYAMNWVRQAPGKGLEWVARIRSKYNNY |
| | -VH | | ATYYADSVKGRFTISRDDSKNTAYLQMNNLKTEDTAVYYCVRHGNFGNSYLSYFAYWG |
| | | | QGTLVTVSS |
| 1445 | G4H_LC_Q46K | artificial | QTVVTQEPSLTVSPGGTVTLTCGSSTGAVTSGYYPNWVQKKPGQAPRGLIGGTKFLAP |
| | -VL | | GTPARFSGSLLGGKAALTLSGVQPEDEAEYYCALWYSNRWVFGGGTKLTVL |
| 1446 | G4H_LC_A107L- | artificial | RYAMN |
| | HCDR1 | | |
| 1447 | G4H_LC_A107L- | artificial | RIRSKYNNYATYYADSVKG |
| | HCDR2 | | |
| 1448 | G4H_LC_A107L- | artificial | HGNFGNSYLSYFAY |
| | HCDR3 | | |
| 1449 | G4H_LC_A107L- | artificial | GSSTGAVTSGYYPN |
| | LCDR1 | | |
| 1450 | G4H_LC_A107L- | artificial | GTKFLAP |
| | LCDR2 | | |
| 1451 | G4H_LC_A107L- | artificial | LLWYSNRWV |
| | LCDR3 | | |
| 1452 | G4H_LC_A107L- | artificial | EVQLVESGGGLVQPGGSLKLSCAASGFTFNRYAMNWVRQAPGKGLEWVARIRSKYNNY |
| | VH | | ATYYADSVKGRFTISRDDSKNTAYLQMNNLKTEDTAVYYCVRHGNFGNSYLSYFAYWG |
| | | | QGTLVTVSS |
| 1453 | G4H_LC_A107L- | artificial | QTVVTQEPSLTVSPGGTVTLTCGSSTGAVTSGYYPNWVQQKPGQAPRGLIGGTKFLAP |
| | VL | | GTPARFSGSLLGGKAALTLSGVQPEDEAEYYCLLWYSNRWVFGGGTKLTVL |
| 1454 | G4H_LC_V44I | artificial | EVQLVESGGGLVQPGGSLKLSCAASGFTFNRYAMNWVRQAPGKGLEWVARIRSKYNNY |
| | -VH | | ATYYADSVKGRFTISRDDSKNTAYLQMNNLKTEDTAVYYCVRHGNFGNSYLSYFAYWG |
| | | | QGTLVTVSS |
| 1455 | G4H_LC_V44I | artificial | QTVVTQEPSLTVSPGGTVTLTCGSSTGAVTSGYYPNWIQQKPGQAPRGLIGGTKFLAP |
| | -VL | | GTPARFSGSLLGGKAALTLSGVQPEDEAEYYCALWYSNRWVFGGGTKLTVL |
| 1456 | G4H_LC_V44L | artificial | EVQLVESGGGLVQPGGSLKLSCAASGFTFNRYAMNWVRQAPGKGLEWVARIRSKYNNY |
| | -VH | | ATYYADSVKGRFTISRDDSKNTAYLQMNNLKTEDTAVYYCVRHGNFGNSYLSYFAYWG |
| | | | QGTLVTVSS |
| 1457 | G4H_LC_V44L | artificial | QTVVTQEPSLTVSPGGTVTLTCGSSTGAVTSGYYPNWLQQKPGQAPRGLIGGTKFLAP |
| | -VL | | GTPARFSGSLLGGKAALTLSGVQPEDEAEYYCALWYSNRWVFGGGTKLTVL |
| 1458 | G4H_LC_V44M | artificial | EVQLVESGGGLVQPGGSLKLSCAASGFTFNRYAMNWVRQAPGKGLEWVARIRSKYNNY |
| | -VH | | ATYYADSVKGRFTISRDDSKNTAYLQMNNLKTEDTAVYYCVRHGNFGNSYLSYFAYWG |
| | | | QGTLVTVSS |
| 1459 | G4H_LC_V44M | artificial | QTVVTQEPSLTVSPGGTVTLTCGSSTGAVTSGYYPNWMQQKPGQAPRGLIGGTKFLAP |
| | -VL | | GTPARFSGSLLGGKAALTLSGVQPEDEAEYYCALWYSNRWVFGGGTKLTVL |
| 1460 | G4H_LC_W109Y- | artificial | RYAMN |
| | HCDR1 | | |
| 1461 | G4H_LC_W109Y- | artificial | RIRSKYNNYATYYADSVKG |
| | HCDR2 | | |
| 1462 | G4H_LC_W109Y- | artificial | HGNFGNSYLSYFAY |
| | HCDR3 | | |
| 1463 | G4H_LC_W109Y- | artificial | GSSTGAVTSGYYPN |
| | LCDR1 | | |
| 1464 | G4H_LC_W109Y- | artificial | GTKFLAP |
| | LCDR2 | | |
| 1465 | G4H_LC_W109Y- | artificial | ALYYSNRWV |
| | LCDR3 | | |
| 1466 | G4H_LC_W109Y- | artificial | EVQLVESGGGLVQPGGSLKLSCAASGFTFNRYAMNWVRQAPGKGLEWVARIRSKYNNY |
| | VH | | ATYYADSVKGRFTISRDDSKNTAYLQMNNLKTEDTAVYYCVRHGNFGNSYLSYFAYWG |
| | | | QGTLVTVSS |
| 1467 | G4H_LC_W109Y- | artificial | QTVVTQEPSLTVSPGGTVTLTCGSSTGAVTSGYYPNWVQQKPGQAPRGLIGGTKFLAP |
| | VL | | GTPARFSGSLLGGKAALTLSGVQPEDEAEYYCALYYSNRWVFGGGTKLTVL |
| 1468 | H1E-HCDR1 | artificial | SYAMN |
| 1469 | H1E-HCDR2 | artificial | RIRSKYNNYATYYADSVKG |
| 1470 | H1E-HCDR3 | artificial | HGNFGNSYLSFWAY |
| 1471 | H1E-LCDR1 | artificial | GSSTGAVTSGYYPN |
| 1472 | H1E-LCDR2 | artificial | GTKFLAP |
| 1473 | H1E-LCDR3 | artificial | ALWYSNRWV |
| 1474 | H1E-VH | artificial | EVQLVESGGGLEQPGGSLKLSCAASGFTFNSYAMNWVRQAPGKGLEWVARIRSKYNNY |
| | | | ATYYADSVKGRFTISRDDSKNTAYLQMNNLKTEDTAVYYCVRHGNFGNSYLSFWAYWG |
| | | | QGTLVTVSS |
| 1475 | H1E-VL | artificial | QTVVTQEPSLTVSPGGTVTLTCGSSTGAVTSGYYPNWVQQKPGQAPRGLIGGTKFLAP |
| | | | GTPARFSGSLLGGKAALTLSGVQPEDEAEYYCALWYSNRWVFGGGTKLTVL |
| 1476 | H1E_HC_A89I | artificial | EVQLVESGGGLEQPGGSLKLSCAASGFTFNSYAMNWVRQAPGKGLEWVARIRSKYNNY |
| | -VH | | ATYYADSVKGRFTISRDDSKNTIYLQMNNLKTEDTAVYYCVRHGNFGNSYLSFWAYWG |
| | | | QGTLVTVSS |
| 1477 | H1E_HC_A89I | artificial | QTVVTQEPSLTVSPGGTVTLTCGSSTGAVTSGYYPNWVQQKPGQAPRGLIGGTKFLAP |
| | -VL | | GTPARFSGSLLGGKAALTLSGVQPEDEAEYYCALWYSNRWVFGGGTKLTVL |
| 1478 | H1E_HC_A89L | artificial | EVQLVESGGGLEQPGGSLKLSCAASGFTFNSYAMNWVRQAPGKGLEWVARIRSKYNNY |
| | -VH | | ATYYADSVKGRFTISRDDSKNTLYLQMNNLKTEDTAVYYCVRHGNFGNSYLSFWAYWG |
| | | | QGTLVTVSS |
| 1479 | H1E_HC_A89L | artificial | QTVVTQEPSLTVSPGGTVTLTCGSSTGAVTSGYYPNWVQQKPGQAPRGLIGGTKFLAP |
| | -VL | | GTPARFSGSLLGGKAALTLSGVQPEDEAEYYCALWYSNRWVFGGGTKLTVL |
| 1480 | H1E_HC_A89T | artificial | EVQLVESGGGLEQPGGSLKLSCAASGFTENSYAMNWVRQAPGKGLEWVARIRSKYNNY |
| | -VH | | ATYYADSVKGRFTISRDDSKNTTYLQMNNLKTEDTAVYYCVRHGNFGNSYLSFWAYWG |
| | | | QGTLVTVSS |
| 1481 | H1E_HC_A89T | artificial | QTVVTQEPSLTVSPGGTVTLTCGSSTGAVTSGYYPNWVQQKPGQAPRGLIGGTKFLAP |
| | -VL | | GTPARFSGSLLGGKAALTLSGVQPEDEAEYYCALWYSNRWVFGGGTKLTVL |
| 1482 | H1E_HC_A89V | artificial | EVQLVESGGGLEQPGGSLKLSCAASGFTENSYAMNWVRQAPGKGLEWVARIRSKYNNY |
| | -VH | | ATYYADSVKGRFTISRDDSKNTVYLQMNNLKTEDTAVYYCVRHGNFGNSYLSFWAYWG |
| | | | QGTLVTVSS |
| 1483 | H1E_HC_A89V | artificial | QTVVTQEPSLTVSPGGTVTLTCGSSTGAVTSGYYPNWVQQKPGQAPRGLIGGTKFLAP |
| | -VL | | GTPARFSGSLLGGKAALTLSGVQPEDEAEYYCALWYSNRWVFGGGTKLTVL |
| 1484 | H1E_HC_L52M | artificial | EVQLVESGGGLEQPGGSLKLSCAASGFTFNSYAMNWVRQAPGKGMEWVARIRSKYNNY |
| | -VH | | ATYYADSVKGRFTISRDDSKNTAYLQMNNLKTEDTAVYYCVRHGNFGNSYLSFWAYWG |
| | | | QGTLVTVSS |
| 1485 | H1E_HC_L52M | artificial | QTVVTQEPSLTVSPGGTVTLTCGSSTGAVTSGYYPNWVQQKPGQAPRGLIGGTKFLAP |
| | -VL | | GTPARFSGSLLGGKAALTLSGVQPEDEAEYYCALWYSNRWVFGGGTKLTVL |
| 1486 | H1E_HC_L52M_ | artificial | EVQLVESGGGLEQPGGSLKLSCAASGFTFNSYAMNWVRQAPGKGMEWVARIRSKYNNY |
| | LC_L21I_ | | ATYYADSVKGRFTISRDDSKNTAYLQMNNLKTEDTAVYYCVRHGNFGNSYLSFWAYWG |
| | Q46K_G141S- | | QGTLVTVSS |
| | VH | | |
| 1487 | H1E_HC_L52M_ | artificial | QTVVTQEPSLTVSPGGTVTITCGSSTGAVTSGYYPNWVQKKPGQAPRGLIGGTKFLAP |
| | LC_L21I_ | | GTPARFSGSLLGGKAALTLSGVQPEDEAEYYCALWYSNRWVFGSGTKLTVL |
| | Q46K_G141S- | | |
| | VL | | |
| 1488 | H1E_HC_L52M_ | artificial | EVQLVESGGGLEQPGGSLKLSCAASGFTFNSYAMNWVRQAPGKGMEWVARIRSKYNNY |
| | LC_Q46K- | | ATYYADSVKGRFTISRDDSKNTAYLQMNNLKTEDTAVYYCVRHGNFGNSYLSFWAYWG |
| | VH | | QGTLVTVSS |
| 1489 | H1E_HC_L52M_ | artificial | QTVVTQEPSLTVSPGGTVTLTCGSSTGAVTSGYYPNWVQKKPGQAPRGLIGGTKFLAP |
| | LC_Q46K- | | GTPARFSGSLLGGKAALTLSGVQPEDEAEYYCALWYSNRWVFGGGTKLTVL |
| | VL | | |
| 1490 | H1E_HC_L52M_ | artificial | EVQLVESGGGLEQPGGSLKLSCAASGFTENSYAMNWVRQAPGKGMEWVARIRSKYNNY |
| | LC_Q46K_ | | ATYYADSVKGRFTISRDDSKNTAYLQMNNLKTEDTAVYYCVRHGNFGNSYLSFWAYWG |
| | G141S-VH | | QGTLVTVSS |
| 1491 | H1E_HC_L52M_ | artificial | QTVVTQEPSLTVSPGGTVTLTCGSSTGAVTSGYYPNWVQKKPGQAPRGLIGGTKFLAP |
| | LC_Q46K_ | | GTPARFSGSLLGGKAALTLSGVQPEDEAEYYCALWYSNRWVFGSGTKLTVL |
| | G141S-VL | | |
| 1492 | H1E_HC_L52V | artificial | EVQLVESGGGLEQPGGSLKLSCAASGFTFNSYAMNWVRQAPGKGVEWVARIRSKYNNY |
| | -VH | | ATYYADSVKGRFTISRDDSKNTAYLQMNNLKTEDTAVYYCVRHGNFGNSYLSFWAYWG |
| | | | QGTLVTVSS |
| 1493 | H1E_HC_L52V | artificial | QTVVTQEPSLTVSPGGTVTLTCGSSTGAVTSGYYPNWVQQKPGQAPRGLIGGTKFLAP |
| | -VL | | GTPARFSGSLLGGKAALTLSGVQPEDEAEYYCALWYSNRWVFGGGTKLTVL |
| 1494 | H1E_HC_L52V_ | artificial | EVQLVESGGGLEQPGGSLKLSCAASGFTFNSYAMNWVRQAPGKGVEWVARIRSKYNNY |
| | LC_V44F- | | ATYYADSVKGRFTISRDDSKNTAYLQMNNLKTEDTAVYYCVRHGNFGNSYLSFWAYWG |
| | VH | | QGTLVTVSS |
| 1495 | H1E_HC_L52V_ | artificial | QTVVTQEPSLTVSPGGTVTLTCGSSTGAVTSGYYPNWFQQKPGQAPRGLIGGTKFLAP |
| | LC_V44F- | | GTPARFSGSLLGGKAALTLSGVQPEDEAEYYCALWYSNRWVFGGGTKLTVL |
| | VL | | |
| 1496 | H1E_HC_L52V_ | artificial | EVQLVESGGGLEQPGGSLKLSCAASGFTENSYAMNWVRQAPGKGVEWVARIRSKYNNY |
| | LC_V44Y- | | ATYYADSVKGRFTISRDDSKNTAYLQMNNLKTEDTAVYYCVRHGNFGNSYLSFWAYWG |
| | VH | | QGTLVTVSS |
| 1497 | H1E_HC_L52V_ | artificial | QTVVTQEPSLTVSPGGTVTLTCGSSTGAVTSGYYPNWYQQKPGQAPRGLIGGTKFLAP |
| | LC_V44Y- | | GTPARFSGSLLGGKAALTLSGVQPEDEAEYYCALWYSNRWVFGGGTKLTVL |
| | VL | | |
| 1498 | H1E_HC_M41F_ | artificial | SYAFN |
| | A89V- | | |
| | HCDR1 | | |
| 1499 | H1E_HC_M41F_ | artificial | RIRSKYNNYATYYADSVKG |
| | A89V- | | |
| | HCDR2 | | |
| 1500 | H1E_HC_M41F_ | artificial | HGNFGNSYLSFWAY |
| | A89V- | | |
| | HCDR3 | | |
| 1501 | H1E_HC_M41F_ | artificial | GSSTGAVTSGYYPN |
| | A89V- | | |
| | LCDR1 | | |
| 1502 | H1E_HC_M41F_ | artificial | GTKFLAP |
| | A89V-LCDR2 | | |
| 1503 | H1E_HC_M41F_ | artificial | ALWYSNRWV |
| | A89V- | | |
| | LCDR3 | | |
| 1504 | H1E_HC_M41F_ | artificial | EVQLVESGGGLEQPGGSLKLSCAASGFTFNSYAFNWVRQAPGKGLEWVARIRSKYNNY |
| | A89V-VH | | ATYYADSVKGRFTISRDDSKNTVYLQMNNLKTEDTAVYYCVRHGNFGNSYLSFWAYWG |
| | | | QGTLVTVSS |
| 1505 | H1E_HC_M41F | artificial | QTVVTQEPSLTVSPGGTVTLTCGSSTGAVTSGYYPNWVQQKPGQAPRGLIGGTKFLAP |
| | A89V-VL | | GTPARFSGSLLGGKAALTLSGVQPEDEAEYYCALWYSNRWVFGGGTKLTVL |
| 1506 | H1E_HC_M41I | artificial | SYAIN |
| | -HCDR1 | | |
| 1507 | H1E_HC_M41I | artificial | RIRSKYNNYATYYADSVKG |
| | -HCDR2 | | |
| 1508 | H1E_HC_M41I | artificial | HGNFGNSYLSFWAY |
| | -HCDR3 | | |
| 1509 | H1E_HC_M41I | artificial | GSSTGAVTSGYYPN |
| | -LCDR1 | | |
| 1510 | H1E_HC_M41I | artificial | GTKFLAP |
| | -LCDR2 | | |
| 1511 | H1E_HC_M41I | artificial | ALWYSNRWV |
| | -LCDR3 | | |
| 1512 | H1E_HC_M41I | artificial | EVQLVESGGGLEQPGGSLKLSCAASGFTENSYAINWVRQAPGKGLEWVARIRSKYNNY |
| | -VH | | ATYYADSVKGRFTISRDDSKNTAYLQMNNLKTEDTAVYYCVRHGNFGNSYLSFWAYWG |
| | | | QGTLVTVSS |
| 1513 | H1E_HC_M41I | artificial | QTVVTQEPSLTVSPGGTVTLTCGSSTGAVTSGYYPNWVQQKPGQAPRGLIGGTKFLAP |
| | -VL | | GTPARFSGSLLGGKAALTLSGVQPEDEAEYYCALWYSNRWVFGGGTKLTVL |
| 1514 | H1E_HC_M41I_ | artificial | EVQLVESGGGLEQPGGSLKLSCAASGFTENSYAINWVRQAPGKGLEWVARIRSKYNNY |
| | A89L-VH | | ATYYADSVKGRFTISRDDSKNTLYLQMNNLKTEDTAVYYCVRHGNFGNSYLSFWAYWG |
| | | | QGTLVTVSS |
| 1515 | H1E_HC_M41I_ | artificial | QTVVTQEPSLTVSPGGTVTLTCGSSTGAVTSGYYPNWVQQKPGQAPRGLIGGTKFLAP |
| | A89L-VL | | GTPARFSGSLLGGKAALTLSGVQPEDEAEYYCALWYSNRWVFGGGTKLTVL |
| 1516 | H1E_HC_M41I_ | artificial | EVQLVESGGGLEQPGGSLKLSCAASGFTFNSYAINWVRQAPGKGLEWVARIRSKYNNY |
| | A89V-VH | | ATYYADSVKGRFTISRDDSKNTVYLQMNNLKTEDTAVYYCVRHGNFGNSYLSFWAYWG |
| | | | QGTLVTVSS |
| 1517 | H1E_HC_M41I_ | artificial | QTVVTQEPSLTVSPGGTVTLTCGSSTGAVTSGYYPNWVQQKPGQAPRGLIGGTKFLAP |
| | A89V-VL | | GTPARFSGSLLGGKAALTLSGVQPEDEAEYYCALWYSNRWVFGGGTKLTVL |
| 1518 | H1E_HC_M41I_ | artificial | EVQLVESGGGLEQPGGSLKLSCAASGFTENSYAINWVRQAPGKGLEWVARIRSKYNNY |
| | A89V_LC_ | | ATYYADSVKGRFTISRDDSKNTVYLQMNNLKTEDTAVYYCVRHGNFGNSYLSFWAYWG |
| | L21I_Q46K_ | | QGTLVTVSS |
| | G141S-VH | | |
| 1519 | H1E_HC_M41I_ | artificial | QTVVTQEPSLTVSPGGTVTITCGSSTGAVTSGYYPNWVQKKPGQAPRGLIGGTKFLAP |
| | A89V_LC_ | | GTPARFSGSLLGGKAALTLSGVQPEDEAEYYCALWYSNRWVFGSGTKLTVL |
| | L21I_Q46K_ | | |
| | G141S-VL | | |
| 1520 | H1E_HC_M41I_ | artificial | EVQLVESGGGLEQPGGSLKLSCAASGFTENSYAINWVRQAPGKGLEWVARIRSKYNNY |
| | A89V_LC_ | | ATYYADSVKGRFTISRDDSKNTVYLQMNNLKTEDTAVYYCVRHGNFGNSYLSFWAYWG |
| | Q46K-VH | | QGTLVTVSS |
| 1521 | H1E_HC_M41I_ | artificial | QTVVTQEPSLTVSPGGTVTLTCGSSTGAVTSGYYPNWVQKKPGQAPRGLIGGTKFLAP |
| | A89V_LC_ | | GTPARFSGSLLGGKAALTLSGVQPEDEAEYYCALWYSNRWVFGGGTKLTVL |
| | Q46K-VL | | |
| 1522 | H1E_HC_M41I_ | artificial | EVQLVESGGGLEQPGGSLKLSCAASGFTFNSYAINWVRQAPGKGVEWVARIRSKYNNY |
| | L52V_A89V_ | | ATYYADSVKGRFTISRDDSKNTVYLQMNNLKTEDTAVYYCVRHGNFGNSYLSFWAYWG |
| | LC_L21I_ | | QGTLVTVSS |
| | Q46K_G141S_ | | |
| | FV44-VH | | |
| 1523 | H1E_HC_M41I_ | artificial | QTVVTQEPSLTVSPGGTVTITCGSSTGAVTSGYYPNWFQKKPGQAPRGLIGGTKFLAP |
| | L52V_A89V_ | | GTPARFSGSLLGGKAALTLSGVQPEDEAEYYCALWYSNRWVFGSGTKLTVL |
| | LC_L21I_ | | |
| | Q46K_G141S_ | | |
| | V44F-VL | | |
| 1524 | H1E_HC_M41I_ | artificial | EVQLVESGGGLEQPGGSLKLSCAASGFTENSYAINWVRQAPGKGLEWVARIRSKYNNY |
| | LC_L21I_ | | ATYYADSVKGRFTISRDDSKNTAYLQMNNLKTEDTAVYYCVRHGNFGNSYLSFWAYWG |
| | Q46K_G141S- | | QGTLVTVSS |
| | VH | | |
| 1525 | H1E_HC_M41I_ | artificial | QTVVTQEPSLTVSPGGTVTITCGSSTGAVTSGYYPNWVQKKPGQAPRGLIGGTKFLAP |
| | LC_L21I_ | | GTPARFSGSLLGGKAALTLSGVQPEDEAEYYCALWYSNRWVFGSGTKLTVL |
| | Q46K_G141S- | | |
| | VL | | |
| 1526 | H1E_HC_M41I_ | artificial | EVQLVESGGGLEQPGGSLKLSCAASGFTFNSYAINWVREAPGKGLEWVARIRSKYNNY |
| | Q46E_A89V_ | | ATYYADSVKGRFTISRDDSKNTVYLQMNNLKTEDTAVYYCVRHGNFGNSYLSFWAYWG |
| | LC_L21I_ | | QGTLVTVSS |
| | Q46K_G141S- | | |
| | VH | | |
| 1527 | H1E_HC_M41I_ | artificial | QTVVTQEPSLTVSPGGTVTITCGSSTGAVTSGYYPNWVQKKPGQAPRGLIGGTKFLAP |
| | Q46E_A89V_ | | GTPARFSGSLLGGKAALTLSGVQPEDEAEYYCALWYSNRWVFGSGTKLTVL |
| | LC_L21I_ | | |
| | Q46K_G141S- | | |
| | VL | | |
| 1528 | H1E_HC_M41V | artificial | SYAVN |
| | -HCDR1 | | |
| 1529 | H1E_HC_M41V | artificial | RIRSKYNNYATYYADSVKG |
| | -HCDR2 | | |
| 1530 | H1E_HC_M41V | artificial | HGNFGNSYLSFWAY |
| | -HCDR3 | | |
| 1531 | H1E_HC_M41V | artificial | GSSTGAVTSGYYPN |
| | -LCDR1 | | |
| 1532 | H1E_HC_M41V | artificial | GTKFLAP |
| | -LCDR2 | | |
| 1533 | H1E_HC_M41V | artificial | ALWYSNRWV |
| | -LCDR3 | | |
| 1534 | H1E_HC_M41V | artificial | EVQLVESGGGLEQPGGSLKLSCAASGFTFNSYAVNWVRQAPGKGLEWVARIRSKYNNY |
| | -VH | | ATYYADSVKGRFTISRDDSKNTAYLQMNNLKTEDTAVYYCVRHGNFGNSYLSFWAYWG |
| | | | QGTLVTVSS |
| 1535 | H1E_HC_M41V | artificial | QTVVTQEPSLTVSPGGTVTLTCGSSTGAVTSGYYPNWVQQKPGQAPRGLIGGTKFLAP |
| | -VL | | GTPARFSGSLLGGKAALTLSGVQPEDEAEYYCALWYSNRWVFGGGTKLTVL |
| 1536 | H1E_HC_Q46D | artificial | EVQLVESGGGLEQPGGSLKLSCAASGFTENSYAMNWVRDAPGKGLEWVARIRSKYNNY |
| | -VH | | ATYYADSVKGRFTISRDDSKNTAYLQMNNLKTEDTAVYYCVRHGNFGNSYLSFWAYWG |
| | | | QGTLVTVSS |
| 1537 | H1E_HC_Q46D | artificial | QTVVTQEPSLTVSPGGTVTLTCGSSTGAVTSGYYPNWVQQKPGQAPRGLIGGTKFLAP |
| | -VL | | GTPARFSGSLLGGKAALTLSGVQPEDEAEYYCALWYSNRWVFGGGTKLTVL |
| 1538 | H1E_HC_Q46D_ | artificial | EVQLVESGGGLEQPGGSLKLSCAASGFTFNSYAMNWVRDAPGKGLEWVARIRSKYNNY |
| | LC_L21I_ | | ATYYADSVKGRFTISRDDSKNTAYLQMNNLKTEDTAVYYCVRHGNFGNSYLSFWAYWG |
| | Q46K_G141S- | | QGTLVTVSS |
| | VH | | |
| 1539 | H1E_HC_Q46D_ | artificial | QTVVTQEPSLTVSPGGTVTITCGSSTGAVTSGYYPNWVQKKPGQAPRGLIGGTKFLAP |
| | LC_L21I_ | | GTPARFSGSLLGGKAALTLSGVQPEDEAEYYCALWYSNRWVFGSGTKLTVL |
| | Q46K_G141S- | | |
| | VL | | |
| 1540 | H1E_HC_Q46D_ | artificial | EVQLVESGGGLEQPGGSLKLSCAASGFTFNSYAMNWVRDAPGKGLEWVARIRSKYNNY |
| | LC_L21I_ | | ATYYADSVKGRFTISRDDSKNTAYLQMNNLKTEDTAVYYCVRHGNFGNSYLSFWAYWG |
| | Q46R_G141S- | | QGTLVTVSS |
| | VH | | |
| 1541 | H1E_HC_Q46D_ | artificial | QTVVTQEPSLTVSPGGTVTITCGSSTGAVTSGYYPNWVQRKPGQAPRGLIGGTKFLAP |
| | LC_L21I_ | | GTPARFSGSLLGGKAALTLSGVQPEDEAEYYCALWYSNRWVFGSGTKLTVL |
| | Q46R_G141S- | | |
| | VL | | |
| 1542 | H1E_HC_Q46D_ | artificial | EVQLVESGGGLEQPGGSLKLSCAASGFTFNSYAMNWVRDAPGKGLEWVARIRSKYNNY |
| | LC_Q46K- | | ATYYADSVKGRFTISRDDSKNTAYLQMNNLKTEDTAVYYCVRHGNFGNSYLSFWAYWG |
| | VH | | QGTLVTVSS |
| 1543 | H1E_HC_Q46D_ | artificial | QTVVTQEPSLTVSPGGTVTLTCGSSTGAVTSGYYPNWVQKKPGQAPRGLIGGTKFLAP |
| | LC_Q46K- | | GTPARFSGSLLGGKAALTLSGVQPEDEAEYYCALWYSNRWVFGGGTKLTVL |
| | VL | | |
| 1544 | H1E_HC_Q46D_ | artificial | EVQLVESGGGLEQPGGSLKLSCAASGFTENSYAMNWVRDAPGKGLEWVARIRSKYNNY |
| | LC_Q46K_ | | ATYYADSVKGRFTISRDDSKNTAYLQMNNLKTEDTAVYYCVRHGNFGNSYLSFWAYWG |
| | G141S-VH | | QGTLVTVSS |
| 1545 | H1E_HC_Q46D_ | artificial | QTVVTQEPSLTVSPGGTVTLTCGSSTGAVTSGYYPNWVQKKPGQAPRGLIGGTKFLAP |
| | LC_Q46K_ | | GTPARFSGSLLGGKAALTLSGVQPEDEAEYYCALWYSNRWVFGSGTKLTVL |
| | G141S-VL | | |
| 1546 | H1E_HC_Q46D_ | artificial | EVQLVESGGGLEQPGGSLKLSCAASGFTENSYAMNWVRDAPGKGLEWVARIRSKYNNY |
| | LC_Q46R- | | ATYYADSVKGRFTISRDDSKNTAYLQMNNLKTEDTAVYYCVRHGNFGNSYLSFWAYWG |
| | VH | | QGTLVTVSS |
| 1547 | H1E_HC_Q46D_ | artificial | QTVVTQEPSLTVSPGGTVTLTCGSSTGAVTSGYYPNWVQRKPGQAPRGLIGGTKFLAP |
| | LC_Q46R- | | GTPARFSGSLLGGKAALTLSGVQPEDEAEYYCALWYSNRWVFGGGTKLTVL |
| | VL | | |
| 1548 | H1E_HC_Q46D_ | artificial | EVQLVESGGGLEQPGGSLKLSCAASGFTENSYAMNWVRDAPGKGLEWVARIRSKYNNY |
| | LC_Q46R_ | | ATYYADSVKGRFTISRDDSKNTAYLQMNNLKTEDTAVYYCVRHGNFGNSYLSFWAYWG |
| | G141S-VH | | QGTLVTVSS |
| 1549 | H1E_HC_Q46D_ | artificial | QTVVTQEPSLTVSPGGTVTLTCGSSTGAVTSGYYPNWVQRKPGQAPRGLIGGTKFLAP |
| | LC_Q46R_ | | GTPARFSGSLLGGKAALTLSGVQPEDEAEYYCALWYSNRWVFGSGTKLTVL |
| | G141S-VL | | |
| 1550 | H1E_HC_Q46E | artificial | EVQLVESGGGLEQPGGSLKLSCAASGFTFNSYAMNWVREAPGKGLEWVARIRSKYNNY |
| | -VH | | ATYYADSVKGRFTISRDDSKNTAYLQMNNLKTEDTAVYYCVRHGNFGNSYLSFWAYWG |
| | | | QGTLVTVSS |
| 1551 | H1E_HC_Q46E | artificial | QTVVTQEPSLTVSPGGTVTLTCGSSTGAVTSGYYPNWVQQKPGQAPRGLIGGTKFLAP |
| | -VL | | GTPARFSGSLLGGKAALTLSGVQPEDEAEYYCALWYSNRWVFGGGTKLTVL |
| 1552 | H1E_HC_Q46E_ | artificial | EVQLVESGGGLEQPGGSLKLSCAASGFTFNSYAMNWVREAPGKGLEWVARIRSKYNNY |
| | LC_L21I_ | | ATYYADSVKGRFTISRDDSKNTAYLQMNNLKTEDTAVYYCVRHGNFGNSYLSFWAYWG |
| | Q46K_G141S- | | QGTLVTVSS |
| | VH | | |
| 1553 | H1E_HC_Q46E_ | artificial | QTVVTQEPSLTVSPGGTVTITCGSSTGAVTSGYYPNWVQKKPGQAPRGLIGGTKFLAP |
| | LC_L21I_ | | GTPARFSGSLLGGKAALTLSGVQPEDEAEYYCALWYSNRWVFGSGTKLTVL |
| | Q46K_G141S- | | |
| | VL | | |
| 1554 | H1E_HC_Q46E_ | artificial | EVQLVESGGGLEQPGGSLKLSCAASGFTFNSYAMNWVREAPGKGLEWVARIRSKYNNY |
| | LC_L21I_ | | ATYYADSVKGRFTISRDDSKNTAYLQMNNLKTEDTAVYYCVRHGNFGNSYLSFWAYWG |
| | Q46R_G141S- | | QGTLVTVSS |
| | VH | | |
| 1555 | H1E_HC_Q46E_ | artificial | QTVVTQEPSLTVSPGGTVTITCGSSTGAVTSGYYPNWVQRKPGQAPRGLIGGTKFLAP |
| | LC_L21I_ | | GTPARFSGSLLGGKAALTLSGVQPEDEAEYYCALWYSNRWVFGSGTKLTVL |
| | Q46R_G141S- | | |
| | VL | | |
| 1556 | H1E_HC_Q46E_ | artificial | EVQLVESGGGLEQPGGSLKLSCAASGFTFNSYAMNWVREAPGKGLEWVARIRSKYNNY |
| | LC_Q46K- | | ATYYADSVKGRFTISRDDSKNTAYLQMNNLKTEDTAVYYCVRHGNFGNSYLSFWAYWG |
| | VH | | QGTLVTVSS |
| 1557 | H1E_HC_Q46E_ | artificial | QTVVTQEPSLTVSPGGTVTLTCGSSTGAVTSGYYPNWVQKKPGQAPRGLIGGTKFLAP |
| | LC_Q46K- | | GTPARFSGSLLGGKAALTLSGVQPEDEAEYYCALWYSNRWVFGGGTKLTVL |
| | VL | | |
| 1558 | H1E_HC_Q46E_ | artificial | EVQLVESGGGLEQPGGSLKLSCAASGFTENSYAMNWVREAPGKGLEWVARIRSKYNNY |
| | LC_Q46K_ | | ATYYADSVKGRFTISRDDSKNTAYLQMNNLKTEDTAVYYCVRHGNFGNSYLSFWAYWG |
| | G141S-VH | | QGTLVTVSS |
| 1559 | H1E_HC_Q46E_ | artificial | QTVVTQEPSLTVSPGGTVTLTCGSSTGAVTSGYYPNWVQKKPGQAPRGLIGGTKFLAP |
| | LC_Q46K_ | | GTPARFSGSLLGGKAALTLSGVQPEDEAEYYCALWYSNRWVFGSGTKLTVL |
| | G141S-VL | | |
| 1560 | H1E_HC_Q46E_ | artificial | EVQLVESGGGLEQPGGSLKLSCAASGFTFNSYAMNWVREAPGKGLEWVARIRSKYNNY |
| | LC_Q46R- | | ATYYADSVKGRFTISRDDSKNTAYLQMNNLKTEDTAVYYCVRHGNFGNSYLSFWAYWG |
| | VH | | QGTLVTVSS |
| 1561 | H1E_HC_Q46E_ | artificial | QTVVTQEPSLTVSPGGTVTLTCGSSTGAVTSGYYPNWVQRKPGQAPRGLIGGTKFLAP |
| | LC_Q46R- | | GTPARFSGSLLGGKAALTLSGVQPEDEAEYYCALWYSNRWVFGGGTKLTVL |
| | VL | | |
| 1562 | H1E_HC_Q46E_ | artificial | EVQLVESGGGLEQPGGSLKLSCAASGFTFNSYAMNWVREAPGKGLEWVARIRSKYNNY |
| | LC_Q46R_ | | ATYYADSVKGRFTISRDDSKNTAYLQMNNLKTEDTAVYYCVRHGNFGNSYLSFWAYWG |
| | G141S-VH | | QGTLVTVSS |
| 1563 | H1E_HC_Q46E_ | artificial | QTVVTQEPSLTVSPGGTVTLTCGSSTGAVTSGYYPNWVQRKPGQAPRGLIGGTKFLAP |
| | LC_Q46R_ | | GTPARFSGSLLGGKAALTLSGVQPEDEAEYYCALWYSNRWVFGSGTKLTVL |
| | G141S-VL | | |
| 1564 | H1E_HC_Q46K | artificial | EVQLVESGGGLEQPGGSLKLSCAASGFTENSYAMNWVRKAPGKGLEWVARIRSKYNNY |
| | -VH | | ATYYADSVKGRFTISRDDSKNTAYLQMNNLKTEDTAVYYCVRHGNFGNSYLSFWAYWG |
| | | | QGTLVTVSS |
| 1565 | H1E_HC_Q46K | artificial | QTVVTQEPSLTVSPGGTVTLTCGSSTGAVTSGYYPNWVQQKPGQAPRGLIGGTKFLAP |
| | -VL | | GTPARFSGSLLGGKAALTLSGVQPEDEAEYYCALWYSNRWVFGGGTKLTVL |
| 1566 | H1E_HC_Q46R | artificial | EVQLVESGGGLEQPGGSLKLSCAASGFTFNSYAMNWVRRAPGKGLEWVARIRSKYNNY |
| | -VH | | ATYYADSVKGRFTISRDDSKNTAYLQMNNLKTEDTAVYYCVRHGNFGNSYLSFWAYWG |
| | | | QGTLVTVSS |
| 1567 | H1E_HC_Q46R | artificial | QTVVTQEPSLTVSPGGTVTLTCGSSTGAVTSGYYPNWVQQKPGQAPRGLIGGTKFLAP |
| | -VL | | GTPARFSGSLLGGKAALTLSGVQPEDEAEYYCALWYSNRWVFGGGTKLTVL |
| 1568 | H1E_HC_V107A- | artificial | EVQLVESGGGLEQPGGSLKLSCAASGFTFNSYAMNWVRQAPGKGLEWVARIRSKYNNY |
| | VH | | ATYYADSVKGRFTISRDDSKNTAYLQMNNLKTEDTAVYYCARHGNFGNSYLSFWAYWG |
| | | | QGTLVTVSS |
| 1569 | H1E_HC_V107A- | artificial | QTVVTQEPSLTVSPGGTVTLTCGSSTGAVTSGYYPNWVQQKPGQAPRGLIGGTKFLAP |
| | VL | | GTPARFSGSLLGGKAALTLSGVQPEDEAEYYCALWYSNRWVFGGGTKLTVL |
| 1570 | H1E_HC_V107L- | artificial | EVQLVESGGGLEQPGGSLKLSCAASGFTFNSYAMNWVRQAPGKGLEWVARIRSKYNNY |
| | VH | | ATYYADSVKGRFTISRDDSKNTAYLQMNNLKTEDTAVYYCLRHGNFGNSYLSFWAYWG |
| | | | QGTLVTVSS |
| 1571 | H1E_HC_V107L- | artificial | QTVVTQEPSLTVSPGGTVTLTCGSSTGAVTSGYYPNWVQQKPGQAPRGLIGGTKFLAP |
| | VL | | GTPARFSGSLLGGKAALTLSGVQPEDEAEYYCALWYSNRWVFGGGTKLTVL |
| 1572 | H1E_HC_W136F- | artificial | SYAMN |
| | HCDR1 | | |
| 1573 | H1E_HC_W136F- | artificial | RIRSKYNNYATYYADSVKG |
| | HCDR2 | | |
| 1574 | H1E_HC_W136F- | artificial | HGNFGNSYLSFFAY |
| | HCDR3 | | |
| 1575 | H1E_HC_W136F- | artificial | GSSTGAVTSGYYPN |
| | LCDR1 | | |
| 1576 | H1E_HC_W136F- | artificial | GTKFLAP |
| | LCDR2 | | |
| 1577 | H1E_HC_W136F- | artificial | ALWYSNRWV |
| | LCDR3 | | |
| 1578 | H1E_HC_W136F- | artificial | EVQLVESGGGLEQPGGSLKLSCAASGFTFNSYAMNWVRQAPGKGLEWVARIRSKYNNY |
| | VH | | ATYYADSVKGRFTISRDDSKNTAYLQMNNLKTEDTAVYYCVRHGNFGNSYLSFFAYWG |
| | | | QGTLVTVSS |
| 1579 | H1E_HC_W136F- | artificial | QTVVTQEPSLTVSPGGTVTLTCGSSTGAVTSGYYPNWVQQKPGQAPRGLIGGTKFLAP |
| | VL | | GTPARFSGSLLGGKAALTLSGVQPEDEAEYYCALWYSNRWVFGGGTKLTVL |
| 1580 | H1E_HC_W136F_ | artificial | EVQLVESGGGLEQPGGSLKLSCAASGFTFNSYAMNWVRQAPGKGLEWVARIRSKYNNY |
| | LC_V44I- | | ATYYADSVKGRFTISRDDSKNTAYLQMNNLKTEDTAVYYCVRHGNFGNSYLSFFAYWG |
| | VH | | QGTLVTVSS |
| 1581 | H1E_HC_W136F_ | artificial | QTVVTQEPSLTVSPGGTVTLTCGSSTGAVTSGYYPNWIQQKPGQAPRGLIGGTKFLAP |
| | LC_V44I- | | GTPARFSGSLLGGKAALTLSGVQPEDEAEYYCALWYSNRWVFGGGTKLTVL |
| | VL | | |
| 1582 | H1E_HC_W136F_ | artificial | EVQLVESGGGLEQPGGSLKLSCAASGFTFNSYAMNWVRQAPGKGLEWVARIRSKYNNY |
| | LC_V44L- | | ATYYADSVKGRFTISRDDSKNTAYLQMNNLKTEDTAVYYCVRHGNFGNSYLSFFAYWG |
| | VH | | QGTLVTVSS |
| 1583 | H1E_HC_W136F_ | artificial | QTVVTQEPSLTVSPGGTVTLTCGSSTGAVTSGYYPNWLQQKPGQAPRGLIGGTKFLAP |
| | LC_V44L- | | GTPARFSGSLLGGKAALTLSGVQPEDEAEYYCALWYSNRWVFGGGTKLTVL |
| | VL | | |
| 1584 | H1E_I2E- | artificial | SYAIN |
| | HCDR1 | | |
| 1585 | H1E_I2E- | artificial | RIRSKYNNYATYYADAVKG |
| | HCDR2 | | |
| 1586 | H1E_I2E- | artificial | AGNFGSSYLSFWAY |
| | HCDR3 | | |
| 1587 | H1E_I2E- | artificial | GSSTGAVTSGYYPN |
| | LCDR1 | | |
| 1588 | H1E_I2E- | artificial | GTKFLAP |
| | LCDR2 | | |
| 1589 | H1E_I2E- | artificial | ALWYSNRWV |
| | LCDR3 | | |
| 1590 | H1E_I2E- | artificial | EVQLVESGGGLEQPGGSLKLSCAASGFTFNSYAINWVRQAPGKGLEWVARIRSKYNNY |
| | VH | | ATYYADAVKGRFTISRDDSKNTVYLQMNNLKTEDTAVYYCARAGNFGSSYLSFWAYWG |
| | | | QGTLVTVSS |
| 1591 | H1E_I2E- | artificial | QTVVTQEPSLTVSPGGTVTITCGSSTGAVTSGYYPNWVQKKPGQAPRGLIGGTKFLAP |
| | VL | | GTPARFSGSLSGGKAALTLSGVQPEDEAEYYCALWYSNRWVFGSGTKLTVL |
| 1592 | H1E_I2K- | artificial | SYAMN |
| | HCDR1 | | |
| 1593 | H1E_I2K- | artificial | RIRSKYNNYATYYAEAVKG |
| | HCDR2 | | |
| 1594 | H1E_I2K- | artificial | NENIGTSYLSFWAY |
| | HCDR3 | | |
| 1595 | H1E_I2K- | artificial | GSSTGAVTSGYYPN |
| | LCDR1 | | |
| 1596 | H1E_I2K- | artificial | GTKFLAP |
| | LCDR2 | | |
| 1597 | H1E_I2K- | artificial | ALWYSNRWV |
| | LCDR3 | | |
| 1598 | H1E_I2K- | artificial | EVQLVESGGGLEQPGGSLKLSCAASGFTFNSYAMNWVRQAPGKGLEWVARIRSKYNNY |
| | VH | | ATYYAEAVKGRFTISRDDSKNTVYLQMNNLKTEDTAVYYCVRNENIGTSYLSFWAYWG |
| | | | QGTLVTVSS |
| 1599 | H1E_I2K- | artificial | QTVVTQEPSLTVSPGGTVTITCGSSTGAVTSGYYPNWVQQKPGQAPRGLIGGTKFLAP |
| | VL | | GTPARFSGSLSGGKAALTLSGVQPEDEAEYYCALWYSNRWVFGSGTKLTVL |
| 1600 | H1E_I2L- | artificial | SYAMN |
| | HCDR1 | | |
| 1601 | H1E_I2L- | artificial | RIRSKYNNYATYYADAVKG |
| | HCDR2 | | |
| 1602 | H1E_I2L- | artificial | AGNFGSSYLSFFAY |
| | HCDR3 | | |
| 1603 | H1E_I2L- | artificial | GSSTGAVTSGYYPN |
| | LCDR1 | | |
| 1604 | H1E_I2L- | artificial | GTKFLAP |
| | LCDR2 | | |
| 1605 | H1E_I2L- | artificial | ALYYSNRWV |
| | LCDR3 | | |
| 1606 | H1E_I2L- | artificial | EVQLVESGGGLEQPGGSLKLSCAASGFTFNSYAMNWVRQAPGKGMEWVARIRSKYNNY |
| | VH | | ATYYADAVKGRFTISRDDSKNTLYLQMNNLKTEDTAVYYCVRAGNFGSSYLSFFAYWG |
| | | | QGTLVTVSS |
| 1607 | H1E_I2L- | artificial | QTVVTQEPSLTVSPGGTVTITCGSSTGAVTSGYYPNWIQKKPGQAPRGLIGGTKFLAP |
| | VL | | GTPARFSGSLEGGKAALTLSGVQPEDEAEYYCALYYSNRWVFGSGTKLTVL |
| 1608 | H1E_I2M- | artificial | SYAMN |
| | HCDR1 | | |
| 1609 | H1E_I2M- | artificial | RIRSKYNNYATYYADAVKG |
| | HCDR2 | | |
| 1610 | H1E_I2M- | artificial | AGNFGTSYLSFWAY |
| | HCDR3 | | |
| 1611 | H1E_I2M- | artificial | GSSTGAVTSGYYPN |
| | LCDR1 | | |
| 1612 | H1E_I2M- | artificial | GTKFLAP |
| | LCDR2 | | |
| 1613 | H1E_I2M- | artificial | ALWYSNRWV |
| | LCDR3 | | |
| 1614 | H1E_I2M- | artificial | EVQLVESGGGLEQPGGSLKLSCAASGFTFNSYAMNWVRQAPGKGMEWVARIRSKYNNY |
| | VH | | ATYYADAVKGRFTISRDDSKNTLYLQMNNLKTEDTAVYYCVRAGNFGTSYLSFWAYWG |
| | | | QGTLVTVSS |
| 1615 | H1E_I2M- | artificial | QTVVTQEPSLTVSPGGTVTITCGSSTGAVTSGYYPNWVQKKPGQAPRGLIGGTKFLAP |
| | VL | | GTPARFSGSLSGGKAALTLSGVQPEDEAEYYCALWYSNRWVFGSGTKLTVL |
| 1616 | H1E_I2M2- | artificial | SYAIN |
| | HCDR1 | | |
| 1617 | H1E_I2M2- | artificial | RIRSKYNNYATYYADAVKG |
| | HCDR2 | | |
| 1618 | H1E_I2M2- | artificial | NANFGTSYLSFFAY |
| | HCDR3 | | |
| 1619 | H1E_I2M2- | artificial | GSSTGAVTSGYYPN |
| | LCDR1 | | |
| 1620 | H1E_I2M2- | artificial | GTKFLAP |
| | LCDR2 | | |
| 1621 | H1E_I2M2- | artificial | ALWYSNRWV |
| | LCDR3 | | |
| 1622 | H1E_I2M2- | artificial | EVQLVESGGGLEQPGGSLKLSCAASGFTFNSYAINWVREAPGKGLEWVARIRSKYNNY |
| | VH | | ATYYADAVKGRFTISRDDSKNTAYLQMNNLKTEDTAVYYCVRNANFGTSYLSFFAYWG |
| | | | QGTLVTVSS |
| 1623 | H1E_I2M2- | artificial | QTVVTQEPSLTVSPGGTVTLTCGSSTGAVTSGYYPNWVQKKPGQAPRGLIGGTKFLAP |
| | VL | | GTPARFSGSLLGGKAALTLSGVQPEDEAEYYCALWYSNRWVFGSGTKLTVL |
| 1624 | H1E_LC_G141S- | artificial | EVQLVESGGGLEQPGGSLKLSCAASGFTFNSYAMNWVRQAPGKGLEWVARIRSKYNNY |
| | VH | | ATYYADSVKGRFTISRDDSKNTAYLQMNNLKTEDTAVYYCVRHGNFGNSYLSFWAYWG |
| | | | QGTLVTVSS |
| 1625 | H1E_LC_G141S- | artificial | QTVVTQEPSLTVSPGGTVTLTCGSSTGAVTSGYYPNWVQQKPGQAPRGLIGGTKFLAP |
| | VL | | GTPARFSGSLLGGKAALTLSGVQPEDEAEYYCALWYSNRWVFGSGTKLTVL |
| 1626 | H1E_LC_G141S_ | artificial | EVQLVESGGGLEQPGGSLKLSCAASGFTENSYAMNWVRQAPGKGLEWVARIRSKYNNY |
| | L21I-VH | | ATYYADSVKGRFTISRDDSKNTAYLQMNNLKTEDTAVYYCVRHGNFGNSYLSFWAYWG |
| | | | QGTLVTVSS |
| 1627 | H1E_LC_G141S_ | artificial | QTVVTQEPSLTVSPGGTVTITCGSSTGAVTSGYYPNWVQQKPGQAPRGLIGGTKFLAP |
| | L21I-VL | | GTPARFSGSLLGGKAALTLSGVQPEDEAEYYCALWYSNRWVFGSGTKLTVL |
| 1628 | H1E_LC_L83E | artificial | EVQLVESGGGLEQPGGSLKLSCAASGFTFNSYAMNWVRQAPGKGLEWVARIRSKYNNY |
| | -VH | | ATYYADSVKGRFTISRDDSKNTAYLQMNNLKTEDTAVYYCVRHGNFGNSYLSFWAYWG |
| | | | QGTLVTVSS |
| 1629 | H1E_LC_L83E | artificial | QTVVTQEPSLTVSPGGTVTLTCGSSTGAVTSGYYPNWVQQKPGQAPRGLIGGTKFLAP |
| | -VL | | GTPARFSGSLEGGKAALTLSGVQPEDEAEYYCALWYSNRWVFGGGTKLTVL |
| 1630 | H1E_LC_L83S | artificial | EVQLVESGGGLEQPGGSLKLSCAASGFTFNSYAMNWVRQAPGKGLEWVARIRSKYNNY |
| | -VH | | ATYYADSVKGRFTISRDDSKNTAYLQMNNLKTEDTAVYYCVRHGNFGNSYLSFWAYWG |
| | | | QGTLVTVSS |
| 1631 | H1E_LC_L83S | artificial | QTVVTQEPSLTVSPGGTVTLTCGSSTGAVTSGYYPNWVQQKPGQAPRGLIGGTKFLAP |
| | -VL | | GTPARFSGSLSGGKAALTLSGVQPEDEAEYYCALWYSNRWVFGGGTKLTVL |
| 1632 | H1E_LC_Q46E | artificial | EVQLVESGGGLEQPGGSLKLSCAASGFTFNSYAMNWVRQAPGKGLEWVARIRSKYNNY |
| | -VH | | ATYYADSVKGRFTISRDDSKNTAYLQMNNLKTEDTAVYYCVRHGNFGNSYLSFWAYWG |
| | | | QGTLVTVSS |
| 1633 | H1E_LC_Q46E | artificial | QTVVTQEPSLTVSPGGTVTLTCGSSTGAVTSGYYPNWVQEKPGQAPRGLIGGTKFLAP |
| | -VL | | GTPARFSGSLLGGKAALTLSGVQPEDEAEYYCALWYSNRWVFGGGTKLTVL |
| 1634 | H1E_LC_Q46K | artificial | EVQLVESGGGLEQPGGSLKLSCAASGFTFNSYAMNWVRQAPGKGLEWVARIRSKYNNY |
| | -VH | | ATYYADSVKGRFTISRDDSKNTAYLQMNNLKTEDTAVYYCVRHGNFGNSYLSFWAYWG |
| | | | QGTLVTVSS |
| 1635 | H1E_LC_Q46K | artificial | QTVVTQEPSLTVSPGGTVTLTCGSSTGAVTSGYYPNWVQKKPGQAPRGLIGGTKFLAP |
| | -VL | | GTPARFSGSLLGGKAALTLSGVQPEDEAEYYCALWYSNRWVFGGGTKLTVL |
| 1636 | H1E_LC_A107L- | artificial | SYAMN |
| | HCDR1 | | |
| 1637 | H1E_LC_A107L- | artificial | RIRSKYNNYATYYADSVKG |
| | HCDR2 | | |
| 1638 | H1E_LC_A107L- | artificial | HGNFGNSYLSFWAY |
| | HCDR3 | | |
| 1639 | H1E_LC_A107L- | artificial | GSSTGAVTSGYYPN |
| | LCDR1 | | |
| 1640 | H1E_LC_A107L- | artificial | GTKFLAP |
| | LCDR2 | | |
| 1641 | H1E_LC_A107L- | artificial | LLWYSNRWV |
| | LCDR3 | | |
| 1642 | H1E_LC_A107L- | artificial | EVQLVESGGGLEQPGGSLKLSCAASGFTFNSYAMNWVRQAPGKGLEWVARIRSKYNNY |
| | VH | | ATYYADSVKGRFTISRDDSKNTAYLQMNNLKTEDTAVYYCVRHGNFGNSYLSFWAYWG |
| | | | QGTLVTVSS |
| 1643 | H1E_LC_A107L- | artificial | QTVVTQEPSLTVSPGGTVTLTCGSSTGAVTSGYYPNWVQQKPGQAPRGLIGGTKFLAP |
| | VL | | GTPARFSGSLLGGKAALTLSGVQPEDEAEYYCLLWYSNRWVFGGGTKLTVL |
| 1644 | H1E_LC_V44I | artificial | EVQLVESGGGLEQPGGSLKLSCAASGFTFNSYAMNWVRQAPGKGLEWVARIRSKYNNY |
| | -VH | | ATYYADSVKGRFTISRDDSKNTAYLQMNNLKTEDTAVYYCVRHGNFGNSYLSFWAYWG |
| | | | QGTLVTVSS |
| 1645 | H1E_LC_V44I | artificial | QTVVTQEPSLTVSPGGTVTLTCGSSTGAVTSGYYPNWIQQKPGQAPRGLIGGTKFLAP |
| | -VL | | GTPARFSGSLLGGKAALTLSGVQPEDEAEYYCALWYSNRWVFGGGTKLTVL |
| 1646 | H1E_LC_V44L | artificial | EVQLVESGGGLEQPGGSLKLSCAASGFTFNSYAMNWVRQAPGKGLEWVARIRSKYNNY |
| | -VH | | ATYYADSVKGRFTISRDDSKNTAYLQMNNLKTEDTAVYYCVRHGNFGNSYLSFWAYWG |
| | | | QGTLVTVSS |
| 1647 | H1E_LC_V44L | artificial | QTVVTQEPSLTVSPGGTVTLTCGSSTGAVTSGYYPNWLQQKPGQAPRGLIGGTKFLAP |
| | -VL | | GTPARFSGSLLGGKAALTLSGVQPEDEAEYYCALWYSNRWVFGGGTKLTVL |
| 1648 | H1E_LC_V44M | artificial | EVQLVESGGGLEQPGGSLKLSCAASGFTFNSYAMNWVRQAPGKGLEWVARIRSKYNNY |
| | -VH | | ATYYADSVKGRFTISRDDSKNTAYLQMNNLKTEDTAVYYCVRHGNFGNSYLSFWAYWG |
| | | | QGTLVTVSS |
| 1649 | H1E_LC_V44M | artificial | QTVVTQEPSLTVSPGGTVTLTCGSSTGAVTSGYYPNWMQQKPGQAPRGLIGGTKFLAP |
| | -VL | | GTPARFSGSLLGGKAALTLSGVQPEDEAEYYCALWYSNRWVFGGGTKLTVL |
| 1650 | H1E_LC_W109Y- | artificial | SYAMN |
| | HCDR1 | | |
| 1651 | H1E_LC_W109Y- | artificial | RIRSKYNNYATYYADSVKG |
| | HCDR2 | | |
| 1652 | H1E_LC_W109Y- | artificial | HGNFGNSYLSFWAY |
| | HCDR3 | | |
| 1653 | H1E_LC_W109Y- | artificial | GSSTGAVTSGYYPN |
| | LCDR1 | | |
| 1654 | H1E_LC_W109Y- | artificial | GTKFLAP |
| | LCDR2 | | |
| 1655 | H1E_LC_W109Y- | artificial | ALYYSNRWV |
| | LCDR3 | | |
| 1656 | H1E_LC_W109Y- | artificial | EVQLVESGGGLEQPGGSLKLSCAASGFTFNSYAMNWVRQAPGKGLEWVARIRSKYNNY |
| | VH | | ATYYADSVKGRFTISRDDSKNTAYLQMNNLKTEDTAVYYCVRHGNFGNSYLSFWAYWG |
| | | | QGTLVTVSS |
| 1657 | H1E_LC_W109Y- | artificial | QTVVTQEPSLTVSPGGTVTLTCGSSTGAVTSGYYPNWVQQKPGQAPRGLIGGTKFLAP |
| | VL | | GTPARFSGSLLGGKAALTLSGVQPEDEAEYYCALYYSNRWVFGGGTKLTVL |
| 1658 | H2C-HCDR1 | artificial | KYAMN |
| 1659 | H2C-HCDR2 | artificial | RIRSKYNNYATYYADSVKD |
| 1660 | H2C-HCDR3 | artificial | HGNFGNSYISYWAY |
| 1661 | H2C-LCDR1 | artificial | GSSTGAVTSGYYPN |
| 1662 | H2C-LCDR2 | artificial | GTKFLAP |
| 1663 | H2C-LCDR3 | artificial | ALWYSNRWV |
| 1664 | H2C-VH | artificial | EVQLVESGGGLVQPGGSLKLSCAASGFTFNKYAMNWVRQAPGKGLEWVARIRSKYNNY |
| | | | ATYYADSVKDRFTISRDDSKNTAYLQMNNLKTEDTAVYYCVRHGNFGNSYISYWAYWG |
| | | | QGTLVTVSS |
| 1665 | H2C-VL | artificial | QTVVTQEPSLTVSPGGTVTLTCGSSTGAVTSGYYPNWVQQKPGQAPRGLIGGTKFLAP |
| | | | GTPARFSGSLLGGKAALTLSGVQPEDEAEYYCALWYSNRWVFGGGTKLTVL |
| 1666 | H2C_HC_A89I | artificial | EVQLVESGGGLVQPGGSLKLSCAASGFTFNKYAMNWVRQAPGKGLEWVARIRSKYNNY |
| | -VH | | ATYYADSVKDRFTISRDDSKNTIYLQMNNLKTEDTAVYYCVRHGNFGNSYISYWAYWG |
| | | | QGTLVTVSS |
| 1667 | H2C_HC_A89I | artificial | QTVVTQEPSLTVSPGGTVTLTCGSSTGAVTSGYYPNWVQQKPGQAPRGLIGGTKFLAP |
| | -VL | | GTPARFSGSLLGGKAALTLSGVQPEDEAEYYCALWYSNRWVFGGGTKLTVL |
| 1668 | H2C_HC_A89L | artificial | EVQLVESGGGLVQPGGSLKLSCAASGFTFNKYAMNWVRQAPGKGLEWVARIRSKYNNY |
| | -VH | | ATYYADSVKDRFTISRDDSKNTLYLQMNNLKTEDTAVYYCVRHGNFGNSYISYWAYWG |
| | | | QGTLVTVSS |
| 1669 | H2C_HC_A89L | artificial | QTVVTQEPSLTVSPGGTVTLTCGSSTGAVTSGYYPNWVQQKPGQAPRGLIGGTKFLAP |
| | -VL | | GTPARFSGSLLGGKAALTLSGVQPEDEAEYYCALWYSNRWVFGGGTKLTVL |
| 1670 | H2C_HC_A89T | artificial | EVQLVESGGGLVQPGGSLKLSCAASGFTFNKYAMNWVRQAPGKGLEWVARIRSKYNNY |
| | -VH | | ATYYADSVKDRFTISRDDSKNTTYLQMNNLKTEDTAVYYCVRHGNFGNSYISYWAYWG |
| | | | QGTLVTVSS |
| 1671 | H2C_HC_A89T | artificial | QTVVTQEPSLTVSPGGTVTLTCGSSTGAVTSGYYPNWVQQKPGQAPRGLIGGTKFLAP |
| | -VL | | GTPARFSGSLLGGKAALTLSGVQPEDEAEYYCALWYSNRWVFGGGTKLTVL |
| 1672 | H2C_HC_A89V | artificial | EVQLVESGGGLVQPGGSLKLSCAASGFTFNKYAMNWVRQAPGKGLEWVARIRSKYNNY |
| | -VH | | ATYYADSVKDRFTISRDDSKNTVYLQMNNLKTEDTAVYYCVRHGNFGNSYISYWAYWG |
| | | | QGTLVTVSS |
| 1673 | H2C_HC_A89V | artificial | QTVVTQEPSLTVSPGGTVTLTCGSSTGAVTSGYYPNWVQQKPGQAPRGLIGGTKFLAP |
| | -VL | | GTPARFSGSLLGGKAALTLSGVQPEDEAEYYCALWYSNRWVFGGGTKLTVL |
| 1674 | H2C_HC_L52M | artificial | EVQLVESGGGLVQPGGSLKLSCAASGFTFNKYAMNWVRQAPGKGMEWVARIRSKYNNY |
| | -VH | | ATYYADSVKDRFTISRDDSKNTAYLQMNNLKTEDTAVYYCVRHGNFGNSYISYWAYWG |
| | | | QGTLVTVSS |
| 1675 | H2C_HC_L52M | artificial | QTVVTQEPSLTVSPGGTVTLTCGSSTGAVTSGYYPNWVQQKPGQAPRGLIGGTKFLAP |
| | -VL | | GTPARFSGSLLGGKAALTLSGVQPEDEAEYYCALWYSNRWVFGGGTKLTVL |
| 1676 | H2C_HC_L52M_ | artificial | EVQLVESGGGLVQPGGSLKLSCAASGFTFNKYAMNWVRQAPGKGMEWVARIRSKYNNY |
| | LC_L21I_ | | ATYYADSVKDRFTISRDDSKNTAYLQMNNLKTEDTAVYYCVRHGNFGNSYISYWAYWG |
| | Q46K_G141S- | | QGTLVTVSS |
| | VH | | |
| 1677 | H2C_HC_L52M_ | artificial | QTVVTQEPSLTVSPGGTVTITCGSSTGAVTSGYYPNWVQKKPGQAPRGLIGGTKFLAP |
| | LC_L21I_ | | GTPARFSGSLLGGKAALTLSGVQPEDEAEYYCALWYSNRWVFGSGTKLTVL |
| | Q46K_G141S | | |
| | VL | | |
| 1678 | H2C_HC_L52M_ | artificial | EVQLVESGGGLVQPGGSLKLSCAASGFTFNKYAMNWVRQAPGKGMEWVARIRSKYNNY |
| | LC_Q46K- | | ATYYADSVKDRFTISRDDSKNTAYLQMNNLKTEDTAVYYCVRHGNFGNSYISYWAYWG |
| | VH | | QGTLVTVSS |
| 1679 | H2C_HC_L52M_ | artificial | QTVVTQEPSLTVSPGGTVTLTCGSSTGAVTSGYYPNWVQKKPGQAPRGLIGGTKFLAP |
| | LC_Q46K- | | GTPARFSGSLLGGKAALTLSGVQPEDEAEYYCALWYSNRWVFGGGTKLTVL |
| | VL | | |
| 1680 | H2C_HC_L52M_ | artificial | EVQLVESGGGLVQPGGSLKLSCAASGFTFNKYAMNWVRQAPGKGMEWVARIRSKYNNY |
| | LC_Q46K_ | | ATYYADSVKDRFTISRDDSKNTAYLQMNNLKTEDTAVYYCVRHGNFGNSYISYWAYWG |
| | G141S-VH | | QGTLVTVSS |
| 1681 | H2C_HC_L52M_ | artificial | QTVVTQEPSLTVSPGGTVTLTCGSSTGAVTSGYYPNWVQKKPGQAPRGLIGGTKFLAP |
| | LC_Q46K_ | | GTPARFSGSLLGGKAALTLSGVQPEDEAEYYCALWYSNRWVFGSGTKLTVL |
| | G141S-VL | | |
| 1682 | H2C_HC_L52V_ | artificial | EVQLVESGGGLVQPGGSLKLSCAASGFTFNKYAMNWVRQAPGKGVEWVARIRSKYNNY |
| | -VH | | ATYYADSVKDRFTISRDDSKNTAYLQMNNLKTEDTAVYYCVRHGNFGNSYISYWAYWG |
| | | | QGTLVTVSS |
| 1683 | H2C_HC_L52V | artificial | QTVVTQEPSLTVSPGGTVTLTCGSSTGAVTSGYYPNWVQQKPGQAPRGLIGGTKFLAP |
| | -VL | | GTPARFSGSLLGGKAALTLSGVQPEDEAEYYCALWYSNRWVFGGGTKLTVL |
| 1684 | H2C_HC_L52V_ | artificial | EVQLVESGGGLVQPGGSLKLSCAASGFTFNKYAMNWVRQAPGKGVEWVARIRSKYNNY |
| | LC_V44F- | | ATYYADSVKDRFTISRDDSKNTAYLQMNNLKTEDTAVYYCVRHGNFGNSYISYWAYWG |
| | VH | | QGTLVTVSS |
| 1685 | H2C_HC_L52V_ | artificial | QTVVTQEPSLTVSPGGTVTLTCGSSTGAVTSGYYPNWFQQKPGQAPRGLIGGTKFLAP |
| | LC_V44F- | | GTPARFSGSLLGGKAALTLSGVQPEDEAEYYCALWYSNRWVFGGGTKLTVL |
| | VL | | |
| 1686 | H2C_HC_L52V_ | artificial | EVQLVESGGGLVQPGGSLKLSCAASGFTFNKYAMNWVRQAPGKGVEWVARIRSKYNNY |
| | LC_V44Y- | | ATYYADSVKDRFTISRDDSKNTAYLQMNNLKTEDTAVYYCVRHGNFGNSYISYWAYWG |
| | VH | | QGTLVTVSS |
| 1687 | H2C_HC_L52V_ | artificial | QTVVTQEPSLTVSPGGTVTLTCGSSTGAVTSGYYPNWYQQKPGQAPRGLIGGTKFLAP |
| | LC_V44Y- | | GTPARFSGSLLGGKAALTLSGVQPEDEAEYYCALWYSNRWVFGGGTKLTVL |
| | VL | | |
| 1688 | H2C_HC_M41F_ | artificial | KYAFN |
| | A89V-HCDR1 | | |
| 1689 | H2C_HC_M41F_ | artificial | RIRSKYNNYATYYADSVKD |
| | A89V-HCDR2 | | |
| 1690 | H2C_HC_M41F_ | artificial | HGNFGNSYISYWAY |
| | A89V-HCDR3 | | |
| 1691 | H2C_HC_M41F_ | artificial | GSSTGAVTSGYYPN |
| | A89V- | | |
| | LCDR1 | | |
| 1692 | H2C_HC_M41F_ | artificial | GTKFLAP |
| | A89V- | | |
| | LCDR2 | | |
| 1693 | H2C_HC_M41F_ | artificial | ALWYSNRWV |
| | A89V- | | |
| | LCDR3 | | |
| 1694 | H2C_HC_M41F_ | artificial | EVQLVESGGGLVQPGGSLKLSCAASGFTFNKYAFNWVRQAPGKGLEWVARIRSKYNNY |
| | A89V-VH | | ATYYADSVKDRFTISRDDSKNTVYLQMNNLKTEDTAVYYCVRHGNFGNSYISYWAYWG |
| | | | QGTLVTVSS |
| 1695 | H2C_HC_M41F_ | artificial | QTVVTQEPSLTVSPGGTVTLTCGSSTGAVTSGYYPNWVQQKPGQAPRGLIGGTKFLAP |
| | A89V-VL | | GTPARFSGSLLGGKAALTLSGVQPEDEAEYYCALWYSNRWVFGGGTKLTVL |
| 1696 | H2C_HC_M41I | artificial | KYAIN |
| | -HCDR1 | | |
| 1697 | H2C_HC_M41I | artificial | RIRSKYNNYATYYADSVKD |
| | -HCDR2 | | |
| 1698 | H2C_HC_M41I | artificial | HGNFGNSYISYWAY |
| | -HCDR3 | | |
| 1699 | H2C_HC_M41I | artificial | GSSTGAVTSGYYPN |
| | -LCDR1 | | |
| 1700 | H2C_HC_M41I | artificial | GTKFLAP |
| | -LCDR2 | | |
| 1701 | H2C_HC_M41I | artificial | ALWYSNRWV |
| | -LCDR3 | | |
| 1702 | H2C_HC_M41I | artificial | EVQLVESGGGLVQPGGSLKLSCAASGFTFNKYAINWVRQAPGKGLEWVARIRSKYNNY |
| | -VH | | ATYYADSVKDRFTISRDDSKNTAYLQMNNLKTEDTAVYYCVRHGNFGNSYISYWAYWG |
| | | | QGTLVTVSS |
| 1703 | H2C_HC_M41I | artificial | QTVVTQEPSLTVSPGGTVTLTCGSSTGAVTSGYYPNWVQQKPGQAPRGLIGGTKFLAP |
| | -VL | | GTPARFSGSLLGGKAALTLSGVQPEDEAEYYCALWYSNRWVFGGGTKLTVL |
| 1704 | H2C_HC_M41I_ | artificial | EVQLVESGGGLVQPGGSLKLSCAASGFTFNKYAINWVRQAPGKGLEWVARIRSKYNNY |
| | A89L-VH | | ATYYADSVKDRFTISRDDSKNTLYLQMNNLKTEDTAVYYCVRHGNFGNSYISYWAYWG |
| | | | QGTLVTVSS |
| 1705 | H2C_HC_M41I_ | artificial | QTVVTQEPSLTVSPGGTVTLTCGSSTGAVTSGYYPNWVQQKPGQAPRGLIGGTKFLAP |
| | A89L-VL | | GTPARFSGSLLGGKAALTLSGVQPEDEAEYYCALWYSNRWVFGGGTKLTVL |
| 1706 | H2C_HC_M41I_ | artificial | EVQLVESGGGLVQPGGSLKLSCAASGFTENKYAINWVRQAPGKGLEWVARIRSKYNNY |
| | A89V-VH | | ATYYADSVKDRFTISRDDSKNTVYLQMNNLKTEDTAVYYCVRHGNFGNSYISYWAYWG |
| | | | QGTLVTVSS |
| 1707 | H2C_HC_M41I_ | artificial | QTVVTQEPSLTVSPGGTVTLTCGSSTGAVTSGYYPNWVQQKPGQAPRGLIGGTKFLAP |
| | A89V-VL | | GTPARFSGSLLGGKAALTLSGVQPEDEAEYYCALWYSNRWVFGGGTKLTVL |
| 1708 | H2C_HC_M41I_ | artificial | EVQLVESGGGLVQPGGSLKLSCAASGFTFNKYAINWVRQAPGKGLEWVARIRSKYNNY |
| | A89V_LC_ | | ATYYADSVKDRFTISRDDSKNTVYLQMNNLKTEDTAVYYCVRHGNFGNSYISYWAYWG |
| | L21I_Q46K_ | | QGTLVTVSS |
| | G141S-VH | | |
| 1709 | H2C_HC_M41I_ | artificial | QTVVTQEPSLTVSPGGTVTITCGSSTGAVTSGYYPNWVQKKPGQAPRGLIGGTKFLAP |
| | A89V_LC_ | | GTPARFSGSLLGGKAALTLSGVQPEDEAEYYCALWYSNRWVFGSGTKLTVL |
| | L21I_Q46K_ | | |
| | G141S-VL | | |
| 1710 | H2C_HC_M41I_ | artificial | EVQLVESGGGLVQPGGSLKLSCAASGFTFNKYAINWVRQAPGKGLEWVARIRSKYNNY |
| | A89V_LC_ | | ATYYADSVKDRFTISRDDSKNTVYLQMNNLKTEDTAVYYCVRHGNFGNSYISYWAYWG |
| | Q46K-VH | | QGTLVTVSS |
| 1711 | H2C_HC_M41I_ | artificial | QTVVTQEPSLTVSPGGTVTLTCGSSTGAVTSGYYPNWVQKKPGQAPRGLIGGTKFLAP |
| | A89V_LC_ | | GTPARFSGSLLGGKAALTLSGVQPEDEAEYYCALWYSNRWVFGGGTKLTVL |
| | Q46K-VL | | |
| 1712 | H2C_HC_M41I_ | artificial | EVQLVESGGGLVQPGGSLKLSCAASGFTFNKYAINWVRQAPGKGVEWVARIRSKYNNY |
| | L52V_A89V_ | | ATYYADSVKDRFTISRDDSKNTVYLQMNNLKTEDTAVYYCVRHGNFGNSYISYWAYWG |
| | LC_L21I_ | | QGTLVTVSS |
| | Q46K_G141S_ | | |
| | V44F-VH | | |
| 1713 | H2C_HC_M41I_ | artificial | QTVVTQEPSLTVSPGGTVTITCGSSTGAVTSGYYPNWFQKKPGQAPRGLIGGTKFLAP |
| | L52V_A89_ | | GTPARFSGSLLGGKAALTLSGVQPEDEAEYYCALWYSNRWVFGSGTKLTVL |
| | LC_L21I_ | | |
| | Q46K_G141S_ | | |
| | V44F-VL | | |
| 1714 | H2C_HC_M41I_ | artificial | EVQLVESGGGLVQPGGSLKLSCAASGFTFNKYAINWVRQAPGKGLEWVARIRSKYNNY |
| | LC_L21I_ | | ATYYADSVKDRFTISRDDSKNTAYLQMNNLKTEDTAVYYCVRHGNFGNSYISYWAYWG |
| | Q46K_G141S- | | QGTLVTVSS |
| | VH | | |
| 1715 | H2C_HC_M41I_ | artificial | QTVVTQEPSLTVSPGGTVTITCGSSTGAVTSGYYPNWVQKKPGQAPRGLIGGTKFLAP |
| | LC_L21I_ | | GTPARFSGSLLGGKAALTLSGVQPEDEAEYYCALWYSNRWVFGSGTKLTVL |
| | Q46K_G141S- | | |
| | VL | | |
| 1716 | H2C_HC_M41I_ | artificial | EVQLVESGGGLVQPGGSLKLSCAASGFTFNKYAINWVREAPGKGLEWVARIRSKYNNY |
| | Q46E_A89V_ | | ATYYADSVKDRFTISRDDSKNTVYLQMNNLKTEDTAVYYCVRHGNFGNSYISYWAYWG |
| | LC_L21I_ | | QGTLVTVSS |
| | KQ46_G141S- | | |
| | VH | | |
| 1717 | H2C_HC_M41I_ | artificial | QTVVTQEPSLTVSPGGTVTITCGSSTGAVTSGYYPNWVQKKPGQAPRGLIGGTKFLAP |
| | Q46E_A89V_ | | GTPARFSGSLLGGKAALTLSGVQPEDEAEYYCALWYSNRWVFGSGTKLTVL |
| | LC_L21I_ | | |
| | Q46K_G141S- | | |
| | VL | | |
| 1718 | H2C_HC_M41V | artificial | KYAVN |
| | -HCDR1 | | |
| 1719 | H2C_HC_M41V | artificial | RIRSKYNNYATYYADSVKD |
| | -HCDR2 | | |
| 1720 | H2C_HC_M41V | artificial | HGNFGNSYISYWAY |
| | -HCDR3 | | |
| 1721 | H2C_HC_M41V | artificial | GSSTGAVTSGYYPN |
| | -LCDR1 | | |
| 1722 | H2C_HC_M41V | artificial | GTKFLAP |
| | -LCDR2 | | |
| 1723 | H2C_HC_M41V | artificial | ALWYSNRWV |
| | -LCDR3 | | |
| 1724 | H2C_HC_M41V | artificial | EVQLVESGGGLVQPGGSLKLSCAASGFTFNKYAVNWVRQAPGKGLEWVARIRSKYNNY |
| | -VH | | ATYYADSVKDRFTISRDDSKNTAYLQMNNLKTEDTAVYYCVRHGNFGNSYISYWAYWG |
| | | | QGTLVTVSS |
| 1725 | H2C_HC_M41V | artificial | QTVVTQEPSLTVSPGGTVTLTCGSSTGAVTSGYYPNWVQQKPGQAPRGLIGGTKFLAP |
| | -VL | | GTPARFSGSLLGGKAALTLSGVQPEDEAEYYCALWYSNRWVFGGGTKLTVL |
| 1726 | H2C_HC_Q46D | artificial | EVQLVESGGGLVQPGGSLKLSCAASGFTFNKYAMNWVRDAPGKGLEWVARIRSKYNNY |
| | -VH | | ATYYADSVKDRFTISRDDSKNTAYLQMNNLKTEDTAVYYCVRHGNFGNSYISYWAYWG |
| | | | QGTLVTVSS |
| 1727 | H2C_HC_Q46D | artificial | QTVVTQEPSLTVSPGGTVTLTCGSSTGAVTSGYYPNWVQQKPGQAPRGLIGGTKFLAP |
| | -VL | | GTPARFSGSLLGGKAALTLSGVQPEDEAEYYCALWYSNRWVFGGGTKLTVL |
| 1728 | H2C_HC_Q46D_ | artificial | EVQLVESGGGLVQPGGSLKLSCAASGFTFNKYAMNWVRDAPGKGLEWVARIRSKYNNY |
| | LC_L21I_ | | ATYYADSVKDRFTISRDDSKNTAYLQMNNLKTEDTAVYYCVRHGNFGNSYISYWAYWG |
| | Q46K_G141S- | | QGTLVTVSS |
| | VH | | |
| 1729 | H2C_HC_Q46D_ | artificial | QTVVTQEPSLTVSPGGTVTITCGSSTGAVTSGYYPNWVQKKPGQAPRGLIGGTKFLAP |
| | LC_L21I_ | | GTPARFSGSLLGGKAALTLSGVQPEDEAEYYCALWYSNRWVFGSGTKLTVL |
| | Q46K_G141S- | | |
| | VL | | |
| 1730 | H2C_HC_Q46D_ | artificial | EVQLVESGGGLVQPGGSLKLSCAASGFTFNKYAMNWVRDAPGKGLEWVARIRSKYNNY |
| | LC_L21I_ | | ATYYADSVKDRFTISRDDSKNTAYLQMNNLKTEDTAVYYCVRHGNFGNSYISYWAYWG |
| | Q46R_G141S- | | QGTLVTVSS |
| | VH | | |
| 1731 | H2C_HC_Q46D_ | artificial | QTVVTQEPSLTVSPGGTVTITCGSSTGAVTSGYYPNWVQRKPGQAPRGLIGGTKFLAP |
| | LC_L21I_ | | GTPARFSGSLLGGKAALTLSGVQPEDEAEYYCALWYSNRWVFGSGTKLTVL |
| | Q46R_G141S- | | |
| | VL | | |
| 1732 | H2C_HC_Q46D_ | artificial | EVQLVESGGGLVQPGGSLKLSCAASGFTENKYAMNWVRDAPGKGLEWVARIRSKYNNY |
| | LC_Q46K- | | ATYYADSVKDRFTISRDDSKNTAYLQMNNLKTEDTAVYYCVRHGNFGNSYISYWAYWG |
| | VH | | QGTLVTVSS |
| 1733 | H2C_HC_Q46D_ | artificial | QTVVTQEPSLTVSPGGTVTLTCGSSTGAVTSGYYPNWVQKKPGQAPRGLIGGTKFLAP |
| | LC_Q46K- | | GTPARFSGSLLGGKAALTLSGVQPEDEAEYYCALWYSNRWVFGGGTKLTVL |
| | VL | | |
| 1734 | H2C_HC_Q46D_ | artificial | EVQLVESGGGLVQPGGSLKLSCAASGFTFNKYAMNWVRDAPGKGLEWVARIRSKYNNY |
| | LC_Q46K_ | | ATYYADSVKDRFTISRDDSKNTAYLQMNNLKTEDTAVYYCVRHGNFGNSYISYWAYWG |
| | G141S-VH | | QGTLVTVSS |
| 1735 | H2C_HC_Q46D_ | artificial | QTVVTQEPSLTVSPGGTVTLTCGSSTGAVTSGYYPNWVQKKPGQAPRGLIGGTKFLAP |
| | LC_Q46K_ | | GTPARFSGSLLGGKAALTLSGVQPEDEAEYYCALWYSNRWVFGSGTKLTVL |
| | 4G11S-VL | | |
| 1736 | H2C_HC_Q46D_ | artificial | EVQLVESGGGLVQPGGSLKLSCAASGFTFNKYAMNWVRDAPGKGLEWVARIRSKYNNY |
| | LC_Q46R- | | ATYYADSVKDRFTISRDDSKNTAYLQMNNLKTEDTAVYYCVRHGNFGNSYISYWAYWG |
| | VH | | QGTLVTVSS |
| 1737 | H2C_HC_Q46D_ | artificial | QTVVTQEPSLTVSPGGTVTLTCGSSTGAVTSGYYPNWVQRKPGQAPRGLIGGTKFLAP |
| | LC_Q46R- | | GTPARFSGSLLGGKAALTLSGVQPEDEAEYYCALWYSNRWVFGGGTKLTVL |
| | VL | | |
| 1738 | H2C_HC_Q46D_ | artificial | EVQLVESGGGLVQPGGSLKLSCAASGFTENKYAMNWVRDAPGKGLEWVARIRSKYNNY |
| | LC_Q46R_ | | ATYYADSVKDRFTISRDDSKNTAYLQMNNLKTEDTAVYYCVRHGNFGNSYISYWAYWG |
| | G141S-VH | | QGTLVTVSS |
| 1739 | H2C_HC_Q46D_ | artificial | QTVVTQEPSLTVSPGGTVTLTCGSSTGAVTSGYYPNWVQRKPGQAPRGLIGGTKFLAP |
| | LC_Q46R_ | | GTPARFSGSLLGGKAALTLSGVQPEDEAEYYCALWYSNRWVFGSGTKLTVL |
| | G141S-VL | | |
| 1740 | H2C_HC_Q46E | artificial | EVQLVESGGGLVQPGGSLKLSCAASGFTFNKYAMNWVREAPGKGLEWVARIRSKYNNY |
| | -VH | | ATYYADSVKDRFTISRDDSKNTAYLQMNNLKTEDTAVYYCVRHGNFGNSYISYWAYWG |
| | | | QGTLVTVSS |
| 1741 | H2C_HC_Q46E | artificial | QTVVTQEPSLTVSPGGTVTLTCGSSTGAVTSGYYPNWVQQKPGQAPRGLIGGTKFLAP |
| | -VL | | GTPARFSGSLLGGKAALTLSGVQPEDEAEYYCALWYSNRWVFGGGTKLTVL |
| 1742 | H2C_HC_Q46E_ | artificial | EVQLVESGGGLVQPGGSLKLSCAASGFTENKYAMNWVREAPGKGLEWVARIRSKYNNY |
| | LC_L21I_ | | ATYYADSVKDRFTISRDDSKNTAYLQMNNLKTEDTAVYYCVRHGNFGNSYISYWAYWG |
| | Q46K_G141S- | | QGTLVTVSS |
| | VH | | |
| 1743 | H2C_HC_Q46E_ | artificial | QTVVTQEPSLTVSPGGTVTITCGSSTGAVTSGYYPNWVQKKPGQAPRGLIGGTKFLAP |
| | LC_L21I_ | | GTPARFSGSLLGGKAALTLSGVQPEDEAEYYCALWYSNRWVFGSGTKLTVL |
| | Q46K_G141S- | | |
| | VL | | |
| 1744 | H2C_HC_Q46E_ | artificial | EVQLVESGGGLVQPGGSLKLSCAASGFTFNKYAMNWVREAPGKGLEWVARIRSKYNNY |
| | LC_L21I_ | | ATYYADSVKDRFTISRDDSKNTAYLQMNNLKTEDTAVYYCVRHGNFGNSYISYWAYWG |
| | 6Q4R_G141S- | | QGTLVTVSS |
| | VH | | |
| 1745 | H2C_HC_Q46E_ | artificial | QTVVTQEPSLTVSPGGTVTITCGSSTGAVTSGYYPNWVQRKPGQAPRGLIGGTKFLAP |
| | LC_L21I_ | | GTPARFSGSLLGGKAALTLSGVQPEDEAEYYCALWYSNRWVFGSGTKLTVL |
| | Q46R_G141S- | | |
| | VL | | |
| 1746 | H2C_HC_Q46E_ | artificial | EVQLVESGGGLVQPGGSLKLSCAASGFTFNKYAMNWVREAPGKGLEWVARIRSKYNNY |
| | LC_Q46K- | | ATYYADSVKDRFTISRDDSKNTAYLQMNNLKTEDTAVYYCVRHGNFGNSYISYWAYWG |
| | VH | | QGTLVTVSS |
| 1747 | H2C_HC_Q46E_ | artificial | QTVVTQEPSLTVSPGGTVTLTCGSSTGAVTSGYYPNWVQKKPGQAPRGLIGGTKFLAP |
| | LC_Q46K- | | GTPARFSGSLLGGKAALTLSGVQPEDEAEYYCALWYSNRWVFGGGTKLTVL |
| | VL | | |
| 1748 | H2C_HC_Q46E_ | artificial | EVQLVESGGGLVQPGGSLKLSCAASGFTFNKYAMNWVREAPGKGLEWVARIRSKYNNY |
| | LC_Q46K_ | | ATYYADSVKDRFTISRDDSKNTAYLQMNNLKTEDTAVYYCVRHGNFGNSYISYWAYWG |
| | G141S-VH | | QGTLVTVSS |
| 1749 | H2C_HC_Q46E_ | artificial | QTVVTQEPSLTVSPGGTVTLTCGSSTGAVTSGYYPNWVQKKPGQAPRGLIGGTKFLAP |
| | LC_Q46K_ | | GTPARFSGSLLGGKAALTLSGVQPEDEAEYYCALWYSNRWVFGSGTKLTVL |
| | G141S-VL | | |
| 1750 | H2C_HC_Q46E_ | artificial | EVQLVESGGGLVQPGGSLKLSCAASGFTFNKYAMNWVREAPGKGLEWVARIRSKYNNY |
| | LC_Q46R- | | ATYYADSVKDRFTISRDDSKNTAYLQMNNLKTEDTAVYYCVRHGNFGNSYISYWAYWG |
| | VH | | QGTLVTVSS |
| 1751 | H2C_HC_Q46E_ | artificial | QTVVTQEPSLTVSPGGTVTLTCGSSTGAVTSGYYPNWVQRKPGQAPRGLIGGTKFLAP |
| | LC_Q46R- | | GTPARFSGSLLGGKAALTLSGVQPEDEAEYYCALWYSNRWVFGGGTKLTVL |
| | VL | | |
| 1752 | H2C_HC_Q46E_ | artificial | EVQLVESGGGLVQPGGSLKLSCAASGFTENKYAMNWVREAPGKGLEWVARIRSKYNNY |
| | LC_Q46R_ | | ATYYADSVKDRFTISRDDSKNTAYLQMNNLKTEDTAVYYCVRHGNFGNSYISYWAYWG |
| | 4G11S-VH | | QGTLVTVSS |
| 1753 | H2C_HC_Q46E_ | artificial | QTVVTQEPSLTVSPGGTVTLTCGSSTGAVTSGYYPNWVQRKPGQAPRGLIGGTKFLAP |
| | LC_Q46R_ | | GTPARFSGSLLGGKAALTLSGVQPEDEAEYYCALWYSNRWVFGSGTKLTVL |
| | G141S-VL | | |
| 1754 | H2C_HC_Q46K | artificial | EVQLVESGGGLVQPGGSLKLSCAASGFTFNKYAMNWVRKAPGKGLEWVARIRSKYNNY |
| | -VH | | ATYYADSVKDRFTISRDDSKNTAYLQMNNLKTEDTAVYYCVRHGNFGNSYISYWAYWG |
| | | | QGTLVTVSS |
| 1755 | H2C_HC_Q46K | artificial | QTVVTQEPSLTVSPGGTVTLTCGSSTGAVTSGYYPNWVQQKPGQAPRGLIGGTKFLAP |
| | -VL | | GTPARFSGSLLGGKAALTLSGVQPEDEAEYYCALWYSNRWVFGGGTKLTVL |
| 1756 | H2C_HC_Q46R | artificial | EVQLVESGGGLVQPGGSLKLSCAASGFTFNKYAMNWVRRAPGKGLEWVARIRSKYNNY |
| | -VH | | ATYYADSVKDRFTISRDDSKNTAYLQMNNLKTEDTAVYYCVRHGNFGNSYISYWAYWG |
| | | | QGTLVTVSS |
| 1757 | H2C_HC_Q46R | artificial | QTVVTQEPSLTVSPGGTVTLTCGSSTGAVTSGYYPNWVQQKPGQAPRGLIGGTKFLAP |
| | -VL | | GTPARFSGSLLGGKAALTLSGVQPEDEAEYYCALWYSNRWVFGGGTKLTVL |
| 1758 | H2C_HC_V107A- | artificial | EVQLVESGGGLVQPGGSLKLSCAASGFTFNKYAMNWVRQAPGKGLEWVARIRSKYNNY |
| | VH | | ATYYADSVKDRFTISRDDSKNTAYLQMNNLKTEDTAVYYCARHGNFGNSYISYWAYWG |
| | | | QGTLVTVSS |
| 1759 | H2C_HC_V107A- | artificial | QTVVTQEPSLTVSPGGTVTLTCGSSTGAVTSGYYPNWVQQKPGQAPRGLIGGTKFLAP |
| | VL | | GTPARFSGSLLGGKAALTLSGVQPEDEAEYYCALWYSNRWVFGGGTKLTVL |
| 1760 | H2C_HC_V107L- | artificial | EVQLVESGGGLVQPGGSLKLSCAASGFTFNKYAMNWVRQAPGKGLEWVARIRSKYNNY |
| | VH | | ATYYADSVKDRFTISRDDSKNTAYLQMNNLKTEDTAVYYCLRHGNFGNSYISYWAYWG |
| | | | QGTLVTVSS |
| 1761 | H2C_HC_V107L- | artificial | QTVVTQEPSLTVSPGGTVTLTCGSSTGAVTSGYYPNWVQQKPGQAPRGLIGGTKFLAP |
| | VL | | GTPARFSGSLLGGKAALTLSGVQPEDEAEYYCALWYSNRWVFGGGTKLTVL |
| 1762 | H2C_HC_W136F- | artificial | KYAMN |
| | HCDR1 | | |
| 1763 | H2C_HC_W136F- | artificial | RIRSKYNNYATYYADSVKD |
| | HCDR2 | | |
| 1764 | H2C_HC_W136F- | artificial | HGNFGNSYISYFAY |
| | HCDR3 | | |
| 1765 | H2C_HC_W136F- | artificial | GSSTGAVTSGYYPN |
| | LCDR1 | | |
| 1766 | H2C_HC_W136F- | artificial | GTKFLAP |
| | LCDR2 | | |
| 1767 | H2C_HC_W136F- | artificial | ALWYSNRWV |
| | LCDR3 | | |
| 1768 | H2C_HC_W136F- | artificial | EVQLVESGGGLVQPGGSLKLSCAASGFTENKYAMNWVRQAPGKGLEWVARIRSKYNNY |
| | VH | | ATYYADSVKDRFTISRDDSKNTAYLQMNNLKTEDTAVYYCVRHGNFGNSYISYFAYWG |
| | | | QGTLVTVSS |
| 1769 | H2C_HC_W136F- | artificial | QTVVTQEPSLTVSPGGTVTLTCGSSTGAVTSGYYPNWVQQKPGQAPRGLIGGTKFLAP |
| | VL | | GTPARFSGSLLGGKAALTLSGVQPEDEAEYYCALWYSNRWVFGGGTKLTVL |
| 1770 | H2C_HC_W136F_ | artificial | EVQLVESGGGLVQPGGSLKLSCAASGFTFNKYAMNWVRQAPGKGLEWVARIRSKYNNY |
| | LC_V44I- | | ATYYADSVKDRFTISRDDSKNTAYLQMNNLKTEDTAVYYCVRHGNFGNSYISYFAYWG |
| | VH | | QGTLVTVSS |
| 1771 | H2C_HC_W136F_ | artificial | QTVVTQEPSLTVSPGGTVTLTCGSSTGAVTSGYYPNWIQQKPGQAPRGLIGGTKFLAP |
| | LC_V44I- | | GTPARFSGSLLGGKAALTLSGVQPEDEAEYYCALWYSNRWVFGGGTKLTVL |
| | VL | | |
| 1772 | H2C_HC_W136F_ | artificial | EVQLVESGGGLVQPGGSLKLSCAASGFTFNKYAMNWVRQAPGKGLEWVARIRSKYNNY |
| | LC_V44L- | | ATYYADSVKDRFTISRDDSKNTAYLQMNNLKTEDTAVYYCVRHGNFGNSYISYFAYWG |
| | VH | | QGTLVTVSS |
| 1773 | H2C_HC_W136F_ | artificial | QTVVTQEPSLTVSPGGTVTLTCGSSTGAVTSGYYPNWLQQKPGQAPRGLIGGTKFLAP |
| | LC_V44L- | | GTPARFSGSLLGGKAALTLSGVQPEDEAEYYCALWYSNRWVFGGGTKLTVL |
| | VL | | |
| 1774 | H2C_I2E- | artificial | KYAIN |
| | HCDR1 | | |
| 1775 | H2C_I2E- | artificial | RIRSKYNNYATYYADAVKD |
| | HCDR2 | | |
| 1776 | H2C_I2E- | artificial | AGNFGSSYISYWAY |
| | HCDR3 | | |
| 1777 | H2C_I2E- | artificial | GSSTGAVTSGYYPN |
| | LCDR1 | | |
| 1778 | H2C_I2E- | artificial | GTKFLAP |
| | LCDR2 | | |
| 1779 | H2C_I2E- | artificial | ALWYSNRWV |
| | LCDR3 | | |
| 1780 | H2C_I2E- | artificial | EVQLVESGGGLVQPGGSLKLSCAASGFTFNKYAINWVRQAPGKGLEWVARIRSKYNNY |
| | VH | | ATYYADAVKDRFTISRDDSKNTVYLQMNNLKTEDTAVYYCARAGNFGSSYISYWAYWG |
| | | | QGTLVTVSS |
| 1781 | H2C_I2E- | artificial | QTVVTQEPSLTVSPGGTVTITCGSSTGAVTSGYYPNWVQKKPGQAPRGLIGGTKFLAP |
| | VL | | GTPARFSGSLSGGKAALTLSGVQPEDEAEYYCALWYSNRWVFGSGTKLTVL |
| 1782 | H2C_I2K- | artificial | KYAMN |
| | HCDR1 | | |
| 1783 | H2C_I2K- | artificial | RIRSKYNNYATYYAEAVKG |
| | HCDR2 | | |
| 1784 | H2C_I2K- | artificial | NENIGTSYISYWAY |
| | HCDR3 | | |
| 1785 | H2C_I2K- | artificial | GSSTGAVTSGYYPN |
| | LCDR1 | | |
| 1786 | H2C_I2K- | artificial | GTKFLAP |
| | LCDR2 | | |
| 1787 | H2C_I2K- | artificial | ALWYSNRWV |
| | LCDR3 | | |
| 1788 | H2C_I2K- | artificial | EVQLVESGGGLVQPGGSLKLSCAASGFTFNKYAMNWVRQAPGKGLEWVARIRSKYNNY |
| | VH | | ATYYAEAVKGRFTISRDDSKNTVYLQMNNLKTEDTAVYYCVRNENIGTSYISYWAYWG |
| | | | QGTLVTVSS |
| 1789 | H2C_I2K- | artificial | QTVVTQEPSLTVSPGGTVTITCGSSTGAVTSGYYPNWVQQKPGQAPRGLIGGTKFLAP |
| | VL | | GTPARFSGSLSGGKAALTLSGVQPEDEAEYYCALWYSNRWVFGSGTKLTVL |
| 1790 | H2C_I2L- | artificial | KYAMN |
| | HCDR1 | | |
| 1791 | H2C_I2L- | artificial | RIRSKYNNYATYYADAVKD |
| | HCDR2 | | |
| 1792 | H2C_I2L- | artificial | AGNFGSSYISYFAY |
| | HCDR3 | | |
| 1793 | H2C_I2L- | artificial | GSSTGAVTSGYYPN |
| | LCDR1 | | |
| 1794 | H2C_I2L- | artificial | GTKFLAP |
| | LCDR2 | | |
| 1795 | H2C_I2L- | artificial | ALYYSNRWV |
| | LCDR3 | | |
| 1796 | H2C_I2L- | artificial | EVQLVESGGGLVQPGGSLKLSCAASGFTFNKYAMNWVRQAPGKGMEWVARIRSKYNNY |
| | VH | | ATYYADAVKDRFTISRDDSKNTLYLQMNNLKTEDTAVYYCVRAGNFGSSYISYFAYWG |
| | | | QGTLVTVSS |
| 1797 | H2C_I2L- | artificial | QTVVTQEPSLTVSPGGTVTITCGSSTGAVTSGYYPNWIQKKPGQAPRGLIGGTKFLAP |
| | VL | | GTPARFSGSLEGGKAALTLSGVQPEDEAEYYCALYYSNRWVFGSGTKLTVL |
| 1798 | H2C_I2M- | artificial | KYAMN |
| | HCDR1 | | |
| 1799 | H2C_I2M- | artificial | RIRSKYNNYATYYADAVKD |
| | HCDR2 | | |
| 1800 | H2C_I2M- | artificial | AGNFGTSYISYWAY |
| | HCDR3 | | |
| 1801 | H2C_I2M- | artificial | GSSTGAVTSGYYPN |
| | LCDR1 | | |
| 1802 | H2C_I2M- | artificial | GTKFLAP |
| | LCDR2 | | |
| 1803 | H2C_I2M- | artificial | ALWYSNRWV |
| | LCDR3 | | |
| 1804 | H2C_I2M- | artificial | EVQLVESGGGLVQPGGSLKLSCAASGFTFNKYAMNWVRQAPGKGMEWVARIRSKYNNY |
| | VH | | ATYYADAVKDRFTISRDDSKNTLYLQMNNLKTEDTAVYYCVRAGNFGTSYISYWAYWG |
| | | | QGTLVTVSS |
| 1805 | H2C_I2M- | artificial | QTVVTQEPSLTVSPGGTVTITCGSSTGAVTSGYYPNWVQKKPGQAPRGLIGGTKFLAP |
| | VL | | GTPARFSGSLSGGKAALTLSGVQPEDEAEYYCALWYSNRWVFGSGTKLTVL |
| 1806 | H2C_I2M2- | artificial | KYAIN |
| | HCDR1 | | |
| 1807 | H2C_I2M2- | artificial | RIRSKYNNYATYYADAVKD |
| | HCDR2 | | |
| 1808 | H2C_I2M2- | artificial | NANFGTSYISYFAY |
| | HCDR3 | | |
| 1809 | H2C_I2M2- | artificial | GSSTGAVTSGYYPN |
| | LCDR1 | | |
| 1810 | H2C_I2M2- | artificial | GTKFLAP |
| | LCDR2 | | |
| 1811 | H2C_I2M2- | artificial | ALWYSNRWV |
| | LCDR3 | | |
| 1812 | H2C_I2M2- | artificial | EVQLVESGGGLVQPGGSLKLSCAASGFTFNKYAINWVREAPGKGLEWVARIRSKYNNY |
| | VH | | ATYYADAVKDRFTISRDDSKNTAYLQMNNLKTEDTAVYYCVRNANFGTSYISYFAYWG |
| | | | QGTLVTVSS |
| 1813 | H2C_I2M2- | artificial | QTVVTQEPSLTVSPGGTVTLTCGSSTGAVTSGYYPNWVQKKPGQAPRGLIGGTKFLAP |
| | VL | | GTPARFSGSLLGGKAALTLSGVQPEDEAEYYCALWYSNRWVFGSGTKLTVL |
| 1814 | H2C_LC_G141S- | artificial | EVQLVESGGGLVQPGGSLKLSCAASGFTFNKYAMNWVRQAPGKGLEWVARIRSKYNNY |
| | VH | | ATYYADSVKDRFTISRDDSKNTAYLQMNNLKTEDTAVYYCVRHGNFGNSYISYWAYWG |
| | | | QGTLVTVSS |
| 1815 | H2C_LC_G141S- | artificial | QTVVTQEPSLTVSPGGTVTLTCGSSTGAVTSGYYPNWVQQKPGQAPRGLIGGTKFLAP |
| | VL | | GTPARFSGSLLGGKAALTLSGVQPEDEAEYYCALWYSNRWVFGSGTKLTVL |
| 1816 | H2C_LC_G141S_ | artificial | EVQLVESGGGLVQPGGSLKLSCAASGFTFNKYAMNWVRQAPGKGLEWVARIRSKYNNY |
| | L21I-VH | | ATYYADSVKDRFTISRDDSKNTAYLQMNNLKTEDTAVYYCVRHGNFGNSYISYWAYWG |
| | | | QGTLVTVSS |
| 1817 | H2C_LC_G141S_ | artificial | QTVVTQEPSLTVSPGGTVTITCGSSTGAVTSGYYPNWVQQKPGQAPRGLIGGTKFLAP |
| | L21I-VL | | GTPARFSGSLLGGKAALTLSGVQPEDEAEYYCALWYSNRWVFGSGTKLTVL |
| 1818 | H2C_LC_L83E | artificial | EVQLVESGGGLVQPGGSLKLSCAASGFTFNKYAMNWVRQAPGKGLEWVARIRSKYNNY |
| | -VH | | ATYYADSVKDRFTISRDDSKNTAYLQMNNLKTEDTAVYYCVRHGNFGNSYISYWAYWG |
| | | | QGTLVTVSS |
| 1819 | H2C_LC_L83E | artificial | QTVVTQEPSLTVSPGGTVTLTCGSSTGAVTSGYYPNWVQQKPGQAPRGLIGGTKFLAP |
| | -VL | | GTPARFSGSLEGGKAALTLSGVQPEDEAEYYCALWYSNRWVFGGGTKLTVL |
| 1820 | H2C_LC_L83S | artificial | EVQLVESGGGLVQPGGSLKLSCAASGFTFNKYAMNWVRQAPGKGLEWVARIRSKYNNY |
| | -VH | | ATYYADSVKDRFTISRDDSKNTAYLQMNNLKTEDTAVYYCVRHGNFGNSYISYWAYWG |
| | | | QGTLVTVSS |
| 1821 | H2C_LC_L83S | artificial | QTVVTQEPSLTVSPGGTVTLTCGSSTGAVTSGYYPNWVQQKPGQAPRGLIGGTKFLAP |
| | -VL | | GTPARFSGSLSGGKAALTLSGVQPEDEAEYYCALWYSNRWVFGGGTKLTVL |
| 1822 | H2C_LC_Q46E | artificial | EVQLVESGGGLVQPGGSLKLSCAASGFTFNKYAMNWVRQAPGKGLEWVARIRSKYNNY |
| | -VH | | ATYYADSVKDRFTISRDDSKNTAYLQMNNLKTEDTAVYYCVRHGNFGNSYISYWAYWG |
| | | | QGTLVTVSS |
| 1823 | H2C_LC_Q46E | artificial | QTVVTQEPSLTVSPGGTVTLTCGSSTGAVTSGYYPNWVQEKPGQAPRGLIGGTKFLAP |
| | -VL | | GTPARFSGSLLGGKAALTLSGVQPEDEAEYYCALWYSNRWVFGGGTKLTVL |
| 1824 | H2C_LC_Q46K | artificial | EVQLVESGGGLVQPGGSLKLSCAASGFTFNKYAMNWVRQAPGKGLEWVARIRSKYNNY |
| | -VH | | ATYYADSVKDRFTISRDDSKNTAYLQMNNLKTEDTAVYYCVRHGNFGNSYISYWAYWG |
| | | | QGTLVTVSS |
| 1825 | H2C_LC_Q46K | artificial | QTVVTQEPSLTVSPGGTVTLTCGSSTGAVTSGYYPNWVQKKPGQAPRGLIGGTKFLAP |
| | -VL | | GTPARFSGSLLGGKAALTLSGVQPEDEAEYYCALWYSNRWVFGGGTKLTVL |
| 1826 | H2C_LC_A107L- | artificial | KYAMN |
| | HCDR1 | | |
| 1827 | H2C_LC_A107L- | artificial | RIRSKYNNYATYYADSVKD |
| | HCDR2 | | |
| 1828 | H2C_LC_A107L- | artificial | HGNFGNSYISYWAY |
| | HCDR3 | | |
| 1829 | H2C_LC_A107L- | artificial | GSSTGAVTSGYYPN |
| | LCDR1 | | |
| 1830 | H2C_LC_A107L- | artificial | GTKFLAP |
| | LCDR2 | | |
| 1831 | H2C_LC_A107L- | artificial | LLWYSNRWV |
| | LCDR3 | | |
| 1832 | H2C_LC_A107L- | artificial | EVQLVESGGGLVQPGGSLKLSCAASGFTENKYAMNWVRQAPGKGLEWVARIRSKYNNY |
| | VH | | ATYYADSVKDRFTISRDDSKNTAYLQMNNLKTEDTAVYYCVRHGNFGNSYISYWAYWG |
| | | | QGTLVTVSS |
| 1833 | H2C_LC_A107L- | artificial | QTVVTQEPSLTVSPGGTVTLTCGSSTGAVTSGYYPNWVQQKPGQAPRGLIGGTKFLAP |
| | VL | | GTPARFSGSLLGGKAALTLSGVQPEDEAEYYCLLWYSNRWVFGGGTKLTVL |
| 1834 | H2C_LC_V44I | artificial | EVQLVESGGGLVQPGGSLKLSCAASGFTFNKYAMNWVRQAPGKGLEWVARIRSKYNNY |
| | -VH | | ATYYADSVKDRFTISRDDSKNTAYLQMNNLKTEDTAVYYCVRHGNFGNSYISYWAYWG |
| | | | QGTLVTVSS |
| 1835 | H2C_LC_V44I | artificial | QTVVTQEPSLTVSPGGTVTLTCGSSTGAVTSGYYPNWIQQKPGQAPRGLIGGTKFLAP |
| | -VL | | GTPARFSGSLLGGKAALTLSGVQPEDEAEYYCALWYSNRWVFGGGTKLTVL |
| 1836 | H2C_LC_V44L | artificial | EVQLVESGGGLVQPGGSLKLSCAASGFTFNKYAMNWVRQAPGKGLEWVARIRSKYNNY |
| | -VH | | ATYYADSVKDRFTISRDDSKNTAYLQMNNLKTEDTAVYYCVRHGNFGNSYISYWAYWG |
| | | | QGTLVTVSS |
| 1837 | H2C_LC_V44L | artificial | QTVVTQEPSLTVSPGGTVTLTCGSSTGAVTSGYYPNWLQQKPGQAPRGLIGGTKFLAP |
| | -VL | | GTPARFSGSLLGGKAALTLSGVQPEDEAEYYCALWYSNRWVFGGGTKLTVL |
| 1838 | H2C_LC_V44M | artificial | EVQLVESGGGLVQPGGSLKLSCAASGFTFNKYAMNWVRQAPGKGLEWVARIRSKYNNY |
| | -VH | | ATYYADSVKDRFTISRDDSKNTAYLQMNNLKTEDTAVYYCVRHGNFGNSYISYWAYWG |
| | | | QGTLVTVSS |
| 1839 | H2C_LC_V44M | artificial | QTVVTQEPSLTVSPGGTVTLTCGSSTGAVTSGYYPNWMQQKPGQAPRGLIGGTKFLAP |
| | -VL | | GTPARFSGSLLGGKAALTLSGVQPEDEAEYYCALWYSNRWVFGGGTKLTVL |
| 1840 | H2C_LC_W109Y- | artificial | KYAMN |
| | HCDR1 | | |
| 1841 | H2C_LC_W109Y- | artificial | RIRSKYNNYATYYADSVKD |
| | HCDR2 | | |
| 1842 | H2C_LC_W109Y- | artificial | HGNFGNSYISYWAY |
| | HCDR3 | | |
| 1843 | H2C_LC_W109Y- | artificial | GSSTGAVTSGYYPN |
| | LCDR1 | | |
| 1844 | H2C_LC_W109Y- | artificial | GTKFLAP |
| | LCDR2 | | |
| 1845 | H2C_LC_W109Y- | artificial | ALYYSNRWV |
| | LCDR3 | | |
| 1846 | H2C_LC_W109Y- | artificial | EVQLVESGGGLVQPGGSLKLSCAASGFTFNKYAMNWVRQAPGKGLEWVARIRSKYNNY |
| | VH | | ATYYADSVKDRFTISRDDSKNTAYLQMNNLKTEDTAVYYCVRHGNFGNSYISYWAYWG |
| | | | QGTLVTVSS |
| 1847 | H2C_LC_W109Y- | artificial | QTVVTQEPSLTVSPGGTVTLTCGSSTGAVTSGYYPNWVQQKPGQAPRGLIGGTKFLAP |
| | VL | | GTPARFSGSLLGGKAALTLSGVQPEDEAEYYCALYYSNRWVFGGGTKLTVL |
| 1848 | I2C-HCDR1 | artificial | KYAMN |
| 1849 | I2C-HCDR2 | artificial | RIRSKYNNYATYYADSVKD |
| 1850 | I2C-HCDR3 | artificial | HGNFGNSYISYWAY |
| 1851 | I2C-LCDR1 | artificial | GSSTGAVTSGNYPN |
| 1852 | I2C-LCDR2 | artificial | GTKFLAP |
| 1853 | I2C-LCDR3 | artificial | VLWYSNRWV |
| 1854 | I2C-VH | artificial | EVQLVESGGGLVQPGGSLKLSCAASGFTFNKYAMNWVRQAPGKGLEWVARIRSKYNNY |
| | | | ATYYADSVKDRFTISRDDSKNTAYLQMNNLKTEDTAVYYCVRHGNFGNSYISYWAYWG |
| | | | QGTLVTVSS |
| 1855 | I2C-VL | artificial | QTVVTQEPSLTVSPGGTVTLTCGSSTGAVTSGNYPNWVQQKPGQAPRGLIGGTKFLAP |
| | | | GTPARFSGSLLGGKAALTLSGVQPEDEAEYYCVLWYSNRWVFGGGTKLTVL |
| 1856 | I2C_HC_A89I | artificial | EVQLVESGGGLVQPGGSLKLSCAASGFTENKYAMNWVRQAPGKGLEWVARIRSKYNNY |
| | -VH | | ATYYADSVKDRFTISRDDSKNTIYLQMNNLKTEDTAVYYCVRHGNFGNSYISYWAYWG |
| | | | QGTLVTVSS |
| 1857 | I2C_HC_A89I | artificial | QTVVTQEPSLTVSPGGTVTLTCGSSTGAVTSGNYPNWVQQKPGQAPRGLIGGTKFLAP |
| | -VL | | GTPARFSGSLLGGKAALTLSGVQPEDEAEYYCVLWYSNRWVFGGGTKLTVL |
| 1858 | I2C_HC_A89L | artificial | EVQLVESGGGLVQPGGSLKLSCAASGFTFNKYAMNWVRQAPGKGLEWVARIRSKYNNY |
| | -VH | | ATYYADSVKDRFTISRDDSKNTLYLQMNNLKTEDTAVYYCVRHGNFGNSYISYWAYWG |
| | | | QGTLVTVSS |
| 1859 | I2C_HC_A89L | artificial | QTVVTQEPSLTVSPGGTVTLTCGSSTGAVTSGNYPNWVQQKPGQAPRGLIGGTKFLAP |
| | -VL | | GTPARFSGSLLGGKAALTLSGVQPEDEAEYYCVLWYSNRWVFGGGTKLTVL |
| 1860 | I2C_HC_A89T | artificial | EVQLVESGGGLVQPGGSLKLSCAASGFTFNKYAMNWVRQAPGKGLEWVARIRSKYNNY |
| | -VH | | ATYYADSVKDRFTISRDDSKNTTYLQMNNLKTEDTAVYYCVRHGNFGNSYISYWAYWG |
| | | | QGTLVTVSS |
| 1861 | I2C_HC_A89T | artificial | QTVVTQEPSLTVSPGGTVTLTCGSSTGAVTSGNYPNWVQQKPGQAPRGLIGGTKFLAP |
| | -VL | | GTPARFSGSLLGGKAALTLSGVQPEDEAEYYCVLWYSNRWVFGGGTKLTVL |
| 1862 | I2C_HC_A89V | artificial | EVQLVESGGGLVQPGGSLKLSCAASGFTFNKYAMNWVRQAPGKGLEWVARIRSKYNNY |
| | -VH | | ATYYADSVKDRFTISRDDSKNTVYLQMNNLKTEDTAVYYCVRHGNFGNSYISYWAYWG |
| | | | QGTLVTVSS |
| 1863 | I2C_HC_A89V | artificial | QTVVTQEPSLTVSPGGTVTLTCGSSTGAVTSGNYPNWVQQKPGQAPRGLIGGTKFLAP |
| | -VL | | GTPARFSGSLLGGKAALTLSGVQPEDEAEYYCVLWYSNRWVFGGGTKLTVL |
| 1864 | I2C_HC_L52M | artificial | EVQLVESGGGLVQPGGSLKLSCAASGFTFNKYAMNWVRQAPGKGMEWVARIRSKYNNY |
| | -VH | | ATYYADSVKDRFTISRDDSKNTAYLQMNNLKTEDTAVYYCVRHGNFGNSYISYWAYWG |
| | | | QGTLVTVSS |
| 1865 | I2C_HC_L52M | artificial | QTVVTQEPSLTVSPGGTVTLTCGSSTGAVTSGNYPNWVQQKPGQAPRGLIGGTKFLAP |
| | -VL | | GTPARFSGSLLGGKAALTLSGVQPEDEAEYYCVLWYSNRWVFGGGTKLTVL |
| 1866 | I2C_HC_L52M_ | artificial | EVQLVESGGGLVQPGGSLKLSCAASGFTFNKYAMNWVRQAPGKGMEWVARIRSKYNNY |
| | LC_L21I_ | | ATYYADSVKDRFTISRDDSKNTAYLQMNNLKTEDTAVYYCVRHGNFGNSYISYWAYWG |
| | Q46K_G141S- | | QGTLVTVSS |
| | VH | | |
| 1867 | I2C_HC_L52M_ | artificial | QTVVTQEPSLTVSPGGTVTITCGSSTGAVTSGNYPNWVQKKPGQAPRGLIGGTKFLAP |
| | LC_L21I_ | | GTPARFSGSLLGGKAALTLSGVQPEDEAEYYCVLWYSNRWVFGSGTKLTVL |
| | 6Q4K_G141S- | | |
| | VL | | |
| 1868 | I2C_HC_L52M_ | artificial | EVQLVESGGGLVQPGGSLKLSCAASGFTFNKYAMNWVRQAPGKGMEWVARIRSKYNNY |
| | LC_Q46K- | | ATYYADSVKDRFTISRDDSKNTAYLQMNNLKTEDTAVYYCVRHGNFGNSYISYWAYWG |
| | VH | | QGTLVTVSS |
| 1869 | I2C_HC_L52M_ | artificial | QTVVTQEPSLTVSPGGTVTLTCGSSTGAVTSGNYPNWVQKKPGQAPRGLIGGTKFLAP |
| | LC_Q46K- | | GTPARFSGSLLGGKAALTLSGVQPEDEAEYYCVLWYSNRWVFGGGTKLTVL |
| | VL | | |
| 1870 | I2C_HC_L52M_ | artificial | EVQLVESGGGLVQPGGSLKLSCAASGFTFNKYAMNWVRQAPGKGMEWVARIRSKYNNY |
| | LC_Q46K_ | | ATYYADSVKDRFTISRDDSKNTAYLQMNNLKTEDTAVYYCVRHGNFGNSYISYWAYWG |
| | G141S-VH | | QGTLVTVSS |
| 1871 | I2C_HC_L52M_ | artificial | QTVVTQEPSLTVSPGGTVTLTCGSSTGAVTSGNYPNWVQKKPGQAPRGLIGGTKFLAP |
| | LC_Q46K_ | | GTPARFSGSLLGGKAALTLSGVQPEDEAEYYCVLWYSNRWVFGSGTKLTVL |
| | G141S-VL | | |
| 1872 | I2C_HC_L52V | artificial | EVQLVESGGGLVQPGGSLKLSCAASGFTFNKYAMNWVRQAPGKGVEWVARIRSKYNNY |
| | -VH | | ATYYADSVKDRFTISRDDSKNTAYLQMNNLKTEDTAVYYCVRHGNFGNSYISYWAYWG |
| | | | QGTLVTVSS |
| 1873 | I2C_HC_L52V | artificial | QTVVTQEPSLTVSPGGTVTLTCGSSTGAVTSGNYPNWVQQKPGQAPRGLIGGTKFLAP |
| | -VL | | GTPARFSGSLLGGKAALTLSGVQPEDEAEYYCVLWYSNRWVFGGGTKLTVL |
| 1874 | I2C_HC_L52V_ | artificial | EVQLVESGGGLVQPGGSLKLSCAASGFTFNKYAMNWVRQAPGKGVEWVARIRSKYNNY |
| | LC_V44F- | | ATYYADSVKDRFTISRDDSKNTAYLQMNNLKTEDTAVYYCVRHGNFGNSYISYWAYWG |
| | VH | | QGTLVTVSS |
| 1875 | I2C_HC_L52V_ | artificial | QTVVTQEPSLTVSPGGTVTLTCGSSTGAVTSGNYPNWFQQKPGQAPRGLIGGTKFLAP |
| | LC_V44F- | | GTPARFSGSLLGGKAALTLSGVQPEDEAEYYCVLWYSNRWVFGGGTKLTVL |
| | VL | | |
| 1876 | I2C_HC_L52V_ | artificial | EVQLVESGGGLVQPGGSLKLSCAASGFTFNKYAMNWVRQAPGKGVEWVARIRSKYNNY |
| | LC_V44Y- | | ATYYADSVKDRFTISRDDSKNTAYLQMNNLKTEDTAVYYCVRHGNFGNSYISYWAYWG |
| | VH | | QGTLVTVSS |
| 1877 | I2C_HC_L52V_ | artificial | QTVVTQEPSLTVSPGGTVTLTCGSSTGAVTSGNYPNWYQQKPGQAPRGLIGGTKFLAP |
| | LC_V44Y- | | GTPARFSGSLLGGKAALTLSGVQPEDEAEYYCVLWYSNRWVFGGGTKLTVL |
| | VL | | |
| 1878 | I2C_HC_M41F_ | artificial | KYAFN |
| | A89V-HCDR1 | | |
| 1879 | I2C_HC_M41F_ | artificial | RIRSKYNNYATYYADSVKD |
| | A89V- | | |
| | HCDR2 | | |
| 1880 | I2C_HC_M41F_ | artificial | HGNFGNSYISYWAY |
| | A89V- | | |
| | HCDR3 | | |
| 1881 | I2C_HC_M41F_ | artificial | GSSTGAVTSGNYPN |
| | A89V- | | |
| | LCDR1 | | |
| 1882 | I2C_HC_M41F_ | artificial | GTKFLAP |
| | A89V-LCDR2 | | |
| 1883 | I2C_HC_M41F_ | artificial | VLWYSNRWV |
| | A89V-LCDR3 | | |
| 1884 | I2C_HC_M41F_ | artificial | EVQLVESGGGLVQPGGSLKLSCAASGFTFNKYAFNWVRQAPGKGLEWVARIRSKYNNY |
| | A89V-VH | | ATYYADSVKDRFTISRDDSKNTVYLQMNNLKTEDTAVYYCVRHGNFGNSYISYWAYWG |
| | | | QGTLVTVSS |
| 1885 | I2C_HC_M41F_ | artificial | QTVVTQEPSLTVSPGGTVTLTCGSSTGAVTSGNYPNWVQQKPGQAPRGLIGGTKFLAP |
| | A89V-VL | | GTPARFSGSLLGGKAALTLSGVQPEDEAEYYCVLWYSNRWVFGGGTKLTVL |
| 1886 | I2C_HC_M41I | artificial | KYAIN |
| | -HCDR1 | | |
| 1887 | I2C_HC_M41I | artificial | RIRSKYNNYATYYADSVKD |
| | -HCDR2 | | |
| 1888 | I2C_HC_M41I | artificial | HGNFGNSYISYWAY |
| | -HCDR3 | | |
| 1889 | I2C_HC_M41I | artificial | GSSTGAVTSGNYPN |
| | -LCDR1 | | |
| 1890 | I2C_HC_M41I | artificial | GTKFLAP |
| | -LCDR2 | | |
| 1891 | I2C_HC_M41I | artificial | VLWYSNRWV |
| | -LCDR3 | | |
| 1892 | I2C_HC_M41I | artificial | EVQLVESGGGLVQPGGSLKLSCAASGFTFNKYAINWVRQAPGKGLEWVARIRSKYNNY |
| | -VH | | ATYYADSVKDRFTISRDDSKNTAYLQMNNLKTEDTAVYYCVRHGNFGNSYISYWAYWG |
| | | | QGTLVTVSS |
| 1893 | I2C_HC_M41I | artificial | QTVVTQEPSLTVSPGGTVTLTCGSSTGAVTSGNYPNWVQQKPGQAPRGLIGGTKFLAP |
| | -VL | | GTPARFSGSLLGGKAALTLSGVQPEDEAEYYCVLWYSNRWVFGGGTKLTVL |
| 1894 | I2C_HC_M41I_ | artificial | EVQLVESGGGLVQPGGSLKLSCAASGFTFNKYAINWVRQAPGKGLEWVARIRSKYNNY |
| | A89L-VH | | ATYYADSVKDRFTISRDDSKNTLYLQMNNLKTEDTAVYYCVRHGNFGNSYISYWAYWG |
| | | | QGTLVTVSS |
| 1895 | I2C_HC_M41I_ | artificial | QTVVTQEPSLTVSPGGTVTLTCGSSTGAVTSGNYPNWVQQKPGQAPRGLIGGTKFLAP |
| | A89L-VL | | GTPARFSGSLLGGKAALTLSGVQPEDEAEYYCVLWYSNRWVFGGGTKLTVL |
| 1896 | I2C_HC_M41I_ | artificial | EVQLVESGGGLVQPGGSLKLSCAASGFTFNKYAINWVRQAPGKGLEWVARIRSKYNNY |
| | A89V-VH | | ATYYADSVKDRFTISRDDSKNTVYLQMNNLKTEDTAVYYCVRHGNFGNSYISYWAYWG |
| | | | QGTLVTVSS |
| 1897 | I2C_HC_M41I_ | artificial | QTVVTQEPSLTVSPGGTVTLTCGSSTGAVTSGNYPNWVQQKPGQAPRGLIGGTKFLAP |
| | A89V-VL | | GTPARFSGSLLGGKAALTLSGVQPEDEAEYYCVLWYSNRWVFGGGTKLTVL |
| 1898 | I2C_HC_M41I_ | artificial | EVQLVESGGGLVQPGGSLKLSCAASGFTFNKYAINWVRQAPGKGLEWVARIRSKYNNY |
| | A89V_LC_ | | ATYYADSVKDRFTISRDDSKNTVYLQMNNLKTEDTAVYYCVRHGNFGNSYISYWAYWG |
| | L21I_Q46K_ | | QGTLVTVSS |
| | G141S-VH | | |
| 1899 | I2C_HC_M41I_ | artificial | QTVVTQEPSLTVSPGGTVTITCGSSTGAVTSGNYPNWVQKKPGQAPRGLIGGTKFLAP |
| | A89V_LC_ | | GTPARFSGSLLGGKAALTLSGVQPEDEAEYYCVLWYSNRWVFGSGTKLTVL |
| | L21I_Q46K_ | | |
| | G141S-VL | | |
| 1900 | I2C_HC_M41I_ | artificial | EVQLVESGGGLVQPGGSLKLSCAASGFTFNKYAINWVRQAPGKGLEWVARIRSKYNNY |
| | A89V_LC_ | | ATYYADSVKDRFTISRDDSKNTVYLQMNNLKTEDTAVYYCVRHGNFGNSYISYWAYWG |
| | 6Q4K-VH | | QGTLVTVSS |
| 1901 | I2C_HC_M41I_ | artificial | QTVVTQEPSLTVSPGGTVTLTCGSSTGAVTSGNYPNWVQKKPGQAPRGLIGGTKFLAP |
| | A89V_LC_ | | GTPARFSGSLLGGKAALTLSGVQPEDEAEYYCVLWYSNRWVFGGGTKLTVL |
| | Q46K-VL | | |
| 1902 | I2C_HC_M41I_ | artificial | EVQLVESGGGLVQPGGSLKLSCAASGFTENKYAINWVRQAPGKGVEWVARIRSKYNNY |
| | L52V_A89V_ | | ATYYADSVKDRFTISRDDSKNTVYLQMNNLKTEDTAVYYCVRHGNFGNSYISYWAYWG |
| | LC_L21I_ | | QGTLVTVSS |
| | Q46K_G141S_ | | |
| | V44F-VH | | |
| 1903 | I2C_HC_M41I_ | artificial | QTVVTQEPSLTVSPGGTVTITCGSSTGAVTSGNYPNWFQKKPGQAPRGLIGGTKFLAP |
| | L52V_A89V_ | | GTPARFSGSLLGGKAALTLSGVQPEDEAEYYCVLWYSNRWVFGSGTKLTVL |
| | LC_L21I_ | | |
| | Q46K_G141S_ | | |
| | V44F-VL | | |
| 1904 | I2C_HC_M41I_ | artificial | EVQLVESGGGLVQPGGSLKLSCAASGFTFNKYAINWVRQAPGKGLEWVARIRSKYNNY |
| | LC_L21I_ | | ATYYADSVKDRFTISRDDSKNTAYLQMNNLKTEDTAVYYCVRHGNFGNSYISYWAYWG |
| | Q46K_G141S- | | QGTLVTVSS |
| | VH | | |
| 1905 | I2C_HC_M41I_ | artificial | QTVVTQEPSLTVSPGGTVTITCGSSTGAVTSGNYPNWVQKKPGQAPRGLIGGTKFLAP |
| | LC_L21I_ | | GTPARFSGSLLGGKAALTLSGVQPEDEAEYYCVLWYSNRWVFGSGTKLTVL |
| | Q46K_G141S- | | |
| | VL | | |
| 1906 | I2C_HC_M41I_ | artificial | EVQLVESGGGLVQPGGSLKLSCAASGFTFNKYAINWVREAPGKGLEWVARIRSKYNNY |
| | Q46E_A89V_ | | ATYYADSVKDRFTISRDDSKNTVYLQMNNLKTEDTAVYYCVRHGNFGNSYISYWAYWG |
| | LC_L21I_ | | QGTLVTVSS |
| | Q46K_G141S- | | |
| | VH | | |
| 1907 | I2C_HC_M41I_ | artificial | QTVVTQEPSLTVSPGGTVTITCGSSTGAVTSGNYPNWVQKKPGQAPRGLIGGTKFLAP |
| | Q46E_A89V_ | | GTPARFSGSLLGGKAALTLSGVQPEDEAEYYCVLWYSNRWVFGSGTKLTVL |
| | LC_L21I_ | | |
| | Q46K_G141S- | | |
| | VL | | |
| 1908 | I2C_HC_M41V | artificial | KYAVN |
| | -HCDR1 | | |
| 1909 | I2C_HC_M41V | artificial | RIRSKYNNYATYYADSVKD |
| | -HCDR2 | | |
| 1910 | I2C_HC_M41V | artificial | HGNFGNSYISYWAY |
| | -HCDR3 | | |
| 1911 | I2C_HC_M41V | artificial | GSSTGAVTSGNYPN |
| | -LCDR1 | | |
| 1912 | I2C_HC_M41V | artificial | GTKFLAP |
| | -LCDR2 | | |
| 1913 | I2C_HC_M41V | artificial | VLWYSNRWV |
| | -LCDR3 | | |
| 1914 | I2C_HC_M41V | artificial | EVQLVESGGGLVQPGGSLKLSCAASGFTFNKYAVNWVRQAPGKGLEWVARIRSKYNNY |
| | -VH | | ATYYADSVKDRFTISRDDSKNTAYLQMNNLKTEDTAVYYCVRHGNFGNSYISYWAYWG |
| | | | QGTLVTVSS |
| 1915 | I2C_HC_M41V | artificial | QTVVTQEPSLTVSPGGTVTLTCGSSTGAVTSGNYPNWVQQKPGQAPRGLIGGTKFLAP |
| | -VL | | GTPARFSGSLLGGKAALTLSGVQPEDEAEYYCVLWYSNRWVFGGGTKLTVL |
| 1916 | I2C_HC_Q46D | artificial | EVQLVESGGGLVQPGGSLKLSCAASGFTENKYAMNWVRDAPGKGLEWVARIRSKYNNY |
| | -VH | | ATYYADSVKDRFTISRDDSKNTAYLQMNNLKTEDTAVYYCVRHGNFGNSYISYWAYWG |
| | | | QGTLVTVSS |
| 1917 | I2C_HC_Q46D | artificial | QTVVTQEPSLTVSPGGTVTLTCGSSTGAVTSGNYPNWVQQKPGQAPRGLIGGTKFLAP |
| | -VL | | GTPARFSGSLLGGKAALTLSGVQPEDEAEYYCVLWYSNRWVFGGGTKLTVL |
| 1918 | I2C_HC_Q46D_ | artificial | EVQLVESGGGLVQPGGSLKLSCAASGFTFNKYAMNWVRDAPGKGLEWVARIRSKYNNY |
| | LC_L21I_ | | ATYYADSVKDRFTISRDDSKNTAYLQMNNLKTEDTAVYYCVRHGNFGNSYISYWAYWG |
| | Q46K_G141S- | | QGTLVTVSS |
| | VH | | |
| 1919 | I2C_HC_Q46D_ | artificial | QTVVTQEPSLTVSPGGTVTITCGSSTGAVTSGNYPNWVQKKPGQAPRGLIGGTKFLAP |
| | LC_L21I_ | | GTPARFSGSLLGGKAALTLSGVQPEDEAEYYCVLWYSNRWVFGSGTKLTVL |
| | Q46K_G141S- | | |
| | VL | | |
| 1920 | I2C_HC_Q46D_ | artificial | EVQLVESGGGLVQPGGSLKLSCAASGFTFNKYAMNWVRDAPGKGLEWVARIRSKYNNY |
| | LC_Q46K- | | ATYYADSVKDRFTISRDDSKNTAYLQMNNLKTEDTAVYYCVRHGNFGNSYISYWAYWG |
| | VH | | QGTLVTVSS |
| 1921 | I2C_HC_Q46D_ | artificial | QTVVTQEPSLTVSPGGTVTLTCGSSTGAVTSGNYPNWVQKKPGQAPRGLIGGTKFLAP |
| | LC_Q46K- | | GTPARFSGSLLGGKAALTLSGVQPEDEAEYYCVLWYSNRWVFGGGTKLTVL |
| | VL | | |
| 1922 | I2C_HC_Q46D_ | artificial | EVQLVESGGGLVQPGGSLKLSCAASGFTFNKYAMNWVRDAPGKGLEWVARIRSKYNNY |
| | LC_Q46K_ | | ATYYADSVKDRFTISRDDSKNTAYLQMNNLKTEDTAVYYCVRHGNFGNSYISYWAYWG |
| | 4G11S-VH | | QGTLVTVSS |
| 1923 | I2C_HC_Q46D_ | artificial | QTVVTQEPSLTVSPGGTVTLTCGSSTGAVTSGNYPNWVQKKPGQAPRGLIGGTKFLAP |
| | LC_Q46K_ | | GTPARFSGSLLGGKAALTLSGVQPEDEAEYYCVLWYSNRWVFGSGTKLTVL |
| | G141S-VL | | |
| 1924 | I2C_HC_Q46D_ | artificial | EVQLVESGGGLVQPGGSLKLSCAASGFTENKYAMNWVRDAPGKGLEWVARIRSKYNNY |
| | LC_Q46R- | | ATYYADSVKDRFTISRDDSKNTAYLQMNNLKTEDTAVYYCVRHGNFGNSYISYWAYWG |
| | VH | | QGTLVTVSS |
| 1925 | I2C_HC_Q46D_ | artificial | QTVVTQEPSLTVSPGGTVTLTCGSSTGAVTSGNYPNWVQRKPGQAPRGLIGGTKFLAP |
| | LC_Q46R- | | GTPARFSGSLLGGKAALTLSGVQPEDEAEYYCVLWYSNRWVFGGGTKLTVL |
| | VL | | |
| 1926 | I2C_HC_Q46D_ | artificial | EVQLVESGGGLVQPGGSLKLSCAASGFTENKYAMNWVRDAPGKGLEWVARIRSKYNNY |
| | LC_Q46R_ | | ATYYADSVKDRFTISRDDSKNTAYLQMNNLKTEDTAVYYCVRHGNFGNSYISYWAYWG |
| | G141S-VH | | QGTLVTVSS |
| 1927 | I2C_HC_Q46D_ | artificial | QTVVTQEPSLTVSPGGTVTLTCGSSTGAVTSGNYPNWVQRKPGQAPRGLIGGTKFLAP |
| | LC_Q46R_ | | GTPARFSGSLLGGKAALTLSGVQPEDEAEYYCVLWYSNRWVFGSGTKLTVL |
| | G141S-VL | | |
| 1928 | I2C_HC_Q46D_ | artificial | EVQLVESGGGLVQPGGSLKLSCAASGFTFNKYAMNWVRDAPGKGLEWVARIRSKYNNY |
| | LC_L21I_ | | ATYYADSVKDRFTISRDDSKNTAYLQMNNLKTEDTAVYYCVRHGNFGNSYISYWAYWG |
| | Q46R_G141S- | | QGTLVTVSS |
| | VH | | |
| 1929 | I2C_HC_Q46D_ | artificial | QTVVTQEPSLTVSPGGTVTITCGSSTGAVTSGNYPNWVQRKPGQAPRGLIGGTKFLAP |
| | LC_L21I_ | | GTPARFSGSLLGGKAALTLSGVQPEDEAEYYCVLWYSNRWVFGSGTKLTVL |
| | Q46R_G141S- | | |
| | VL | | |
| 1930 | I2C_HC_Q46E | artificial | EVQLVESGGGLVQPGGSLKLSCAASGFTENKYAMNWVREAPGKGLEWVARIRSKYNNY |
| | -VH | | ATYYADSVKDRFTISRDDSKNTAYLQMNNLKTEDTAVYYCVRHGNFGNSYISYWAYWG |
| | | | QGTLVTVSS |
| 1931 | I2C_HC_Q46E | artificial | QTVVTQEPSLTVSPGGTVTLTCGSSTGAVTSGNYPNWVQQKPGQAPRGLIGGTKFLAP |
| | -VL | | GTPARFSGSLLGGKAALTLSGVQPEDEAEYYCVLWYSNRWVFGGGTKLTVL |
| 1932 | I2C_HC_Q46E_ | artificial | EVQLVESGGGLVQPGGSLKLSCAASGFTFNKYAMNWVREAPGKGLEWVARIRSKYNNY |
| | LC_L21I_ | | ATYYADSVKDRFTISRDDSKNTAYLQMNNLKTEDTAVYYCVRHGNFGNSYISYWAYWG |
| | Q46K_G141S- | | QGTLVTVSS |
| | VH | | |
| 1933 | I2C_HC_Q46E_ | artificial | QTVVTQEPSLTVSPGGTVTITCGSSTGAVTSGNYPNWVQKKPGQAPRGLIGGTKFLAP |
| | LC_L21I_ | | GTPARFSGSLLGGKAALTLSGVQPEDEAEYYCVLWYSNRWVFGSGTKLTVL |
| | Q46K_G141S- | | |
| | VL | | |
| 1934 | I2C_HC_Q46E_ | artificial | EVQLVESGGGLVQPGGSLKLSCAASGFTFNKYAMNWVREAPGKGLEWVARIRSKYNNY |
| | LC_L21I_ | | ATYYADSVKDRFTISRDDSKNTAYLQMNNLKTEDTAVYYCVRHGNFGNSYISYWAYWG |
| | Q46R_G141S- | | QGTLVTVSS |
| | VH | | |
| 1935 | I2C_HC_Q46E_ | artificial | QTVVTQEPSLTVSPGGTVTITCGSSTGAVTSGNYPNWVQRKPGQAPRGLIGGTKFLAP |
| | LC_L21I_ | | GTPARFSGSLLGGKAALTLSGVQPEDEAEYYCVLWYSNRWVFGSGTKLTVL |
| | Q46R_G141S- | | |
| | VL | | |
| 1936 | I2C_HC_Q46E_ | artificial | EVQLVESGGGLVQPGGSLKLSCAASGFTFNKYAMNWVREAPGKGLEWVARIRSKYNNY |
| | LC_Q46K- | | ATYYADSVKDRFTISRDDSKNTAYLQMNNLKTEDTAVYYCVRHGNFGNSYISYWAYWG |
| | VH | | QGTLVTVSS |
| 1937 | I2C_HC_Q46E_ | artificial | QTVVTQEPSLTVSPGGTVTLTCGSSTGAVTSGNYPNWVQKKPGQAPRGLIGGTKFLAP |
| | LC_Q46K- | | GTPARFSGSLLGGKAALTLSGVQPEDEAEYYCVLWYSNRWVFGGGTKLTVL |
| | VL | | |
| 1938 | I2C_HC_Q46E_ | artificial | EVQLVESGGGLVQPGGSLKLSCAASGFTENKYAMNWVREAPGKGLEWVARIRSKYNNY |
| | LC_Q46K_ | | ATYYADSVKDRFTISRDDSKNTAYLQMNNLKTEDTAVYYCVRHGNFGNSYISYWAYWG |
| | G141S-VH | | QGTLVTVSS |
| 1939 | I2C_HC_Q46E_ | artificial | QTVVTQEPSLTVSPGGTVTLTCGSSTGAVTSGNYPNWVQKKPGQAPRGLIGGTKFLAP |
| | LC_Q46K_ | | GTPARFSGSLLGGKAALTLSGVQPEDEAEYYCVLWYSNRWVFGSGTKLTVL |
| | G141S-VL | | |
| 1940 | I2C_HC_Q46E_ | artificial | EVQLVESGGGLVQPGGSLKLSCAASGFTENKYAMNWVREAPGKGLEWVARIRSKYNNY |
| | LC_Q46R- | | ATYYADSVKDRFTISRDDSKNTAYLQMNNLKTEDTAVYYCVRHGNFGNSYISYWAYWG |
| | VH | | QGTLVTVSS |
| 1941 | I2C_HC_Q46E_ | artificial | QTVVTQEPSLTVSPGGTVTLTCGSSTGAVTSGNYPNWVQRKPGQAPRGLIGGTKFLAP |
| | LC_Q46R- | | GTPARFSGSLLGGKAALTLSGVQPEDEAEYYCVLWYSNRWVFGGGTKLTVL |
| | VL | | |
| 1942 | I2C_HC_Q46E_ | artificial | EVQLVESGGGLVQPGGSLKLSCAASGFTFNKYAMNWVREAPGKGLEWVARIRSKYNNY |
| | LC_Q46R_ | | ATYYADSVKDRFTISRDDSKNTAYLQMNNLKTEDTAVYYCVRHGNFGNSYISYWAYWG |
| | G141S-VH | | QGTLVTVSS |
| 1943 | I2C_HC_Q46E_ | artificial | QTVVTQEPSLTVSPGGTVTLTCGSSTGAVTSGNYPNWVQRKPGQAPRGLIGGTKFLAP |
| | LC_Q46R_ | | GTPARFSGSLLGGKAALTLSGVQPEDEAEYYCVLWYSNRWVFGSGTKLTVL |
| | G141S-VL | | |
| 1944 | I2C_HC_Q46K | artificial | EVQLVESGGGLVQPGGSLKLSCAASGFTFNKYAMNWVRKAPGKGLEWVARIRSKYNNY |
| | -VH | | ATYYADSVKDRFTISRDDSKNTAYLQMNNLKTEDTAVYYCVRHGNFGNSYISYWAYWG |
| | | | QGTLVTVSS |
| 1945 | I2C_HC_Q46K | artificial | QTVVTQEPSLTVSPGGTVTLTCGSSTGAVTSGNYPNWVQQKPGQAPRGLIGGTKFLAP |
| | -VL | | GTPARFSGSLLGGKAALTLSGVQPEDEAEYYCVLWYSNRWVFGGGTKLTVL |
| 1946 | I2C_HC_Q46R | artificial | EVQLVESGGGLVQPGGSLKLSCAASGFTFNKYAMNWVRRAPGKGLEWVARIRSKYNNY |
| | -VH | | ATYYADSVKDRFTISRDDSKNTAYLQMNNLKTEDTAVYYCVRHGNFGNSYISYWAYWG |
| | | | QGTLVTVSS |
| 1947 | I2C_HC_Q46R | artificial | QTVVTQEPSLTVSPGGTVTLTCGSSTGAVTSGNYPNWVQQKPGQAPRGLIGGTKFLAP |
| | -VL | | GTPARFSGSLLGGKAALTLSGVQPEDEAEYYCVLWYSNRWVFGGGTKLTVL |
| 1948 | I2C_HC_V107A- | artificial | EVQLVESGGGLVQPGGSLKLSCAASGFTFNKYAMNWVRQAPGKGLEWVARIRSKYNNY |
| | VH | | ATYYADSVKDRFTISRDDSKNTAYLQMNNLKTEDTAVYYCARHGNFGNSYISYWAYWG |
| | | | QGTLVTVSS |
| 1949 | I2C_HC_V107A- | artificial | QTVVTQEPSLTVSPGGTVTLTCGSSTGAVTSGNYPNWVQQKPGQAPRGLIGGTKFLAP |
| | VL | | GTPARFSGSLLGGKAALTLSGVQPEDEAEYYCVLWYSNRWVFGGGTKLTVL |
| 1950 | I2C_HC_V107L- | artificial | EVQLVESGGGLVQPGGSLKLSCAASGFTFNKYAMNWVRQAPGKGLEWVARIRSKYNNY |
| | VH | | ATYYADSVKDRFTISRDDSKNTAYLQMNNLKTEDTAVYYCLRHGNFGNSYISYWAYWG |
| | | | QGTLVTVSS |
| 1951 | I2C_HC_V107L- | artificial | QTVVTQEPSLTVSPGGTVTLTCGSSTGAVTSGNYPNWVQQKPGQAPRGLIGGTKFLAP |
| | VL | | GTPARFSGSLLGGKAALTLSGVQPEDEAEYYCVLWYSNRWVFGGGTKLTVL |
| 1952 | I2C_HC_W136F- | artificial | KYAMN |
| | HCDR1 | | |
| 1953 | I2C_HC_W136F- | artificial | RIRSKYNNYATYYADSVKD |
| | HCDR2 | | |
| 1954 | I2C_HC_W136F- | artificial | HGNFGNSYISYFAY |
| | HCDR3 | | |
| 1955 | I2C_HC_W136F- | artificial | GSSTGAVTSGNYPN |
| | LCDR1 | | |
| 1956 | I2C_HC_W136F- | artificial | GTKFLAP |
| | LCDR2 | | |
| 1957 | I2C_HC_W136F- | artificial | VLWYSNRWV |
| | LCDR3 | | |
| 1958 | I2C_HC_W136F- | artificial | EVQLVESGGGLVQPGGSLKLSCAASGFTFNKYAMNWVRQAPGKGLEWVARIRSKYNNY |
| | VH | | ATYYADSVKDRFTISRDDSKNTAYLQMNNLKTEDTAVYYCVRHGNFGNSYISYFAYWG |
| | | | QGTLVTVSS |
| 1959 | I2C_HC_W136F- | artificial | QTVVTQEPSLTVSPGGTVTLTCGSSTGAVTSGNYPNWVQQKPGQAPRGLIGGTKFLAP |
| | VL | | GTPARFSGSLLGGKAALTLSGVQPEDEAEYYCVLWYSNRWVFGGGTKLTVL |
| 1960 | I2C_HC_W136F_ | artificial | EVQLVESGGGLVQPGGSLKLSCAASGFTFNKYAMNWVRQAPGKGLEWVARIRSKYNNY |
| | LC_V44I- | | ATYYADSVKDRFTISRDDSKNTAYLQMNNLKTEDTAVYYCVRHGNFGNSYISYFAYWG |
| | VH | | QGTLVTVSS |
| 1961 | I2C_HC_W136F_ | artificial | QTVVTQEPSLTVSPGGTVTLTCGSSTGAVTSGNYPNWIQQKPGQAPRGLIGGTKFLAP |
| | LC_V44I- | | GTPARFSGSLLGGKAALTLSGVQPEDEAEYYCVLWYSNRWVFGGGTKLTVL |
| | VL | | |
| 1962 | I2C_HC_W136F_ | artificial | EVQLVESGGGLVQPGGSLKLSCAASGFTFNKYAMNWVRQAPGKGLEWVARIRSKYNNY |
| | LC_V44L- | | ATYYADSVKDRFTISRDDSKNTAYLQMNNLKTEDTAVYYCVRHGNFGNSYISYFAYWG |
| | VH | | QGTLVTVSS |
| 1963 | I2C_HC_W136F_ | artificial | QTVVTQEPSLTVSPGGTVTLTCGSSTGAVTSGNYPNWLQQKPGQAPRGLIGGTKFLAP |
| | LC_V44L- | | GTPARFSGSLLGGKAALTLSGVQPEDEAEYYCVLWYSNRWVFGGGTKLTVL |
| | VL | | |
| 1964 | I2C_LC_G141S- | artificial | EVQLVESGGGLVQPGGSLKLSCAASGFTFNKYAMNWVRQAPGKGLEWVARIRSKYNNY |
| | VH | | ATYYADSVKDRFTISRDDSKNTAYLQMNNLKTEDTAVYYCVRHGNFGNSYISYWAYWG |
| | | | QGTLVTVSS |
| 1965 | I2C_LC_G141S- | artificial | QTVVTQEPSLTVSPGGTVTLTCGSSTGAVTSGNYPNWVQQKPGQAPRGLIGGTKFLAP |
| | VL | | GTPARFSGSLLGGKAALTLSGVQPEDEAEYYCVLWYSNRWVFGSGTKLTVL |
| 1966 | I2C_LC_G141S_ | artificial | EVQLVESGGGLVQPGGSLKLSCAASGFTFNKYAMNWVRQAPGKGLEWVARIRSKYNNY |
| | L21I-VH | | ATYYADSVKDRFTISRDDSKNTAYLQMNNLKTEDTAVYYCVRHGNFGNSYISYWAYWG |
| | | | QGTLVTVSS |
| 1967 | I2C_LC_G141S_ | artificial | QTVVTQEPSLTVSPGGTVTITCGSSTGAVTSGNYPNWVQQKPGQAPRGLIGGTKFLAP |
| | L21I-VL | | GTPARFSGSLLGGKAALTLSGVQPEDEAEYYCVLWYSNRWVFGSGTKLTVL |
| 1968 | I2C_LC_L83E | artificial | EVQLVESGGGLVQPGGSLKLSCAASGFTFNKYAMNWVRQAPGKGLEWVARIRSKYNNY |
| | -VH | | ATYYADSVKDRFTISRDDSKNTAYLQMNNLKTEDTAVYYCVRHGNFGNSYISYWAYWG |
| | | | QGTLVTVSS |
| 1969 | I2C_LC_L83E | artificial | QTVVTQEPSLTVSPGGTVTLTCGSSTGAVTSGNYPNWVQQKPGQAPRGLIGGTKFLAP |
| | -VL | | GTPARFSGSLEGGKAALTLSGVQPEDEAEYYCVLWYSNRWVFGGGTKLTVL |
| 1970 | I2C_LC_L83S | artificial | EVQLVESGGGLVQPGGSLKLSCAASGFTFNKYAMNWVRQAPGKGLEWVARIRSKYNNY |
| | -VH | | ATYYADSVKDRFTISRDDSKNTAYLQMNNLKTEDTAVYYCVRHGNFGNSYISYWAYWG |
| | | | QGTLVTVSS |
| 1971 | I2C_LC_L83S | artificial | QTVVTQEPSLTVSPGGTVTLTCGSSTGAVTSGNYPNWVQQKPGQAPRGLIGGTKFLAP |
| | -VL | | GTPARFSGSLSGGKAALTLSGVQPEDEAEYYCVLWYSNRWVFGGGTKLTVL |
| 1972 | I2C_LC_Q46E | artificial | EVQLVESGGGLVQPGGSLKLSCAASGFTFNKYAMNWVRQAPGKGLEWVARIRSKYNNY |
| | -VH | | ATYYADSVKDRFTISRDDSKNTAYLQMNNLKTEDTAVYYCVRHGNFGNSYISYWAYWG |
| | | | QGTLVTVSS |
| 1973 | I2C_LC_Q46E | artificial | QTVVTQEPSLTVSPGGTVTLTCGSSTGAVTSGNYPNWVQEKPGQAPRGLIGGTKFLAP |
| | -VL | | GTPARFSGSLLGGKAALTLSGVQPEDEAEYYCVLWYSNRWVFGGGTKLTVL |
| 1974 | I2C_LC_Q46K | artificial | EVQLVESGGGLVQPGGSLKLSCAASGFTFNKYAMNWVRQAPGKGLEWVARIRSKYNNY |
| | -VH | | ATYYADSVKDRFTISRDDSKNTAYLQMNNLKTEDTAVYYCVRHGNFGNSYISYWAYWG |
| | | | QGTLVTVSS |
| 1975 | I2C_LC_Q46K | artificial | QTVVTQEPSLTVSPGGTVTLTCGSSTGAVTSGNYPNWVQKKPGQAPRGLIGGTKFLAP |
| | -VL | | GTPARFSGSLLGGKAALTLSGVQPEDEAEYYCVLWYSNRWVFGGGTKLTVL |
| 1976 | I2C_LC_V107A- | artificial | KYAMN |
| | HCDR1 | | |
| 1977 | I2C_LC_V107A- | artificial | RIRSKYNNYATYYADSVKD |
| | HCDR2 | | |
| 1978 | I2C_LC_V107A- | artificial | HGNFGNSYISYWAY |
| | HCDR3 | | |
| 1979 | I2C_LC_V107A- | artificial | GSSTGAVTSGNYPN |
| | LCDR1 | | |
| 1980 | I2C_LC_V107A- | artificial | GTKFLAP |
| | LCDR2 | | |
| 1981 | I2C_LC_V107A- | artificial | ALWYSNRWV |
| | LCDR3 | | |
| 1982 | I2C_LC_V107A- | artificial | EVQLVESGGGLVQPGGSLKLSCAASGFTFNKYAMNWVRQAPGKGLEWVARIRSKYNNY |
| | VH | | ATYYADSVKDRFTISRDDSKNTAYLQMNNLKTEDTAVYYCVRHGNFGNSYISYWAYWG |
| | | | QGTLVTVSS |
| 1983 | I2C_LC_V107A- | artificial | QTVVTQEPSLTVSPGGTVTLTCGSSTGAVTSGNYPNWVQQKPGQAPRGLIGGTKFLAP |
| | VL | | GTPARFSGSLLGGKAALTLSGVQPEDEAEYYCALWYSNRWVFGGGTKLTVL |
| 1984 | I2C_LC_V107L- | artificial | KYAMN |
| | HCDR1 | | |
| 1985 | I2C_LC_V107L- | artificial | RIRSKYNNYATYYADSVKD |
| | HCDR2 | | |
| 1986 | I2C_LC_V107L- | artificial | HGNFGNSYISYWAY |
| | HCDR3 | | |
| 1987 | I2C_LC_V107L- | artificial | GSSTGAVTSGNYPN |
| | LCDR1 | | |
| 1988 | I2C_LC_V107L- | artificial | GTKFLAP |
| | LCDR2 | | |
| 1989 | I2C_LC_V107L- | artificial | LLWYSNRWV |
| | LCDR3 | | |
| 1990 | I2C_LC_V107L- | artificial | EVQLVESGGGLVQPGGSLKLSCAASGFTFNKYAMNWVRQAPGKGLEWVARIRSKYNNY |
| | VH | | ATYYADSVKDRFTISRDDSKNTAYLQMNNLKTEDTAVYYCVRHGNFGNSYISYWAYWG |
| | | | QGTLVTVSS |
| 1991 | I2C_LC_V107L- | artificial | QTVVTQEPSLTVSPGGTVTLTCGSSTGAVTSGNYPNWVQQKPGQAPRGLIGGTKFLAP |
| | VL | | GTPARFSGSLLGGKAALTLSGVQPEDEAEYYCLLWYSNRWVFGGGTKLTVL |
| 1992 | I2C_LC_V44I | artificial | EVQLVESGGGLVQPGGSLKLSCAASGFTFNKYAMNWVRQAPGKGLEWVARIRSKYNNY |
| | -VH | | ATYYADSVKDRFTISRDDSKNTAYLQMNNLKTEDTAVYYCVRHGNFGNSYISYWAYWG |
| | | | QGTLVTVSS |
| 1993 | I2C_LC_V44I | artificial | QTVVTQEPSLTVSPGGTVTLTCGSSTGAVTSGNYPNWIQQKPGQAPRGLIGGTKFLAP |
| | -VL | | GTPARFSGSLLGGKAALTLSGVQPEDEAEYYCVLWYSNRWVFGGGTKLTVL |
| 1994 | I2C_LC_V44L | artificial | EVQLVESGGGLVQPGGSLKLSCAASGFTENKYAMNWVRQAPGKGLEWVARIRSKYNNY |
| | -VH | | ATYYADSVKDRFTISRDDSKNTAYLQMNNLKTEDTAVYYCVRHGNFGNSYISYWAYWG |
| | | | QGTLVTVSS |
| 1995 | I2C_LC_V44L | artificial | QTVVTQEPSLTVSPGGTVTLTCGSSTGAVTSGNYPNWLQQKPGQAPRGLIGGTKFLAP |
| | -VL | | GTPARFSGSLLGGKAALTLSGVQPEDEAEYYCVLWYSNRWVFGGGTKLTVL |
| 1996 | I2C_LC_V44M | artificial | EVQLVESGGGLVQPGGSLKLSCAASGFTFNKYAMNWVRQAPGKGLEWVARIRSKYNNY |
| | -VH | | ATYYADSVKDRFTISRDDSKNTAYLQMNNLKTEDTAVYYCVRHGNFGNSYISYWAYWG |
| | | | QGTLVTVSS |
| 1997 | I2C_LC_V44M | artificial | QTVVTQEPSLTVSPGGTVTLTCGSSTGAVTSGNYPNWMQQKPGQAPRGLIGGTKFLAP |
| | -VL | | GTPARFSGSLLGGKAALTLSGVQPEDEAEYYCVLWYSNRWVFGGGTKLTVL |
| 1998 | I2C_LC_W109Y- | artificial | KYAMN |
| | HCDR1 | | |
| 1999 | I2C_LC_W109Y- | artificial | RIRSKYNNYATYYADSVKD |
| | HCDR2 | | |
| 2000 | I2C_LC_W109Y- | artificial | HGNFGNSYISYWAY |
| | HCDR3 | | |
| 2001 | I2C_LC_W109Y- | artificial | GSSTGAVTSGNYPN |
| | LCDR1 | | |
| 2002 | I2C_LC_W109Y- | artificial | GTKFLAP |
| | LCDR2 | | |
| 2003 | I2C_LC_W109Y- | artificial | VLYYSNRWV |
| | LCDR3 | | |
| 2004 | I2C_LC_W109Y- | artificial | EVQLVESGGGLVQPGGSLKLSCAASGFTFNKYAMNWVRQAPGKGLEWVARIRSKYNNY |
| | VH | | ATYYADSVKDRFTISRDDSKNTAYLQMNNLKTEDTAVYYCVRHGNFGNSYISYWAYWG |
| | | | QGTLVTVSS |
| 2005 | I2C_LC_W109Y- | artificial | QTVVTQEPSLTVSPGGTVTLTCGSSTGAVTSGNYPNWVQQKPGQAPRGLIGGTKFLAP |
| | VL | | GTPARFSGSLLGGKAALTLSGVQPEDEAEYYCVLYYSNRWVFGGGTKLTVL |
| 2006 | I2E-HCDR1 | artificial | KYAIN |
| 2007 | I2E-HCDR2 | artificial | RIRSKYNNYATYYADAVKD |
| 2008 | I2E-HCDR3 | artificial | AGNFGSSYISYWAY |
| 2009 | I2E-LCDR1 | artificial | GSSTGAVTSGNYPN |
| 2010 | I2E-LCDR2 | artificial | GTKFLAP |
| 2011 | I2E-LCDR3 | artificial | VLWYSNRWV |
| 2012 | I2E-VH | artificial | EVQLVESGGGLVQPGGSLKLSCAASGFTFNKYAINWVRQAPGKGLEWVARIRSKYNNY |
| | | | ATYYADAVKDRFTISRDDSKNTVYLQMNNLKTEDTAVYYCARAGNFGSSYISYWAYWG |
| | | | QGTLVTVSS |
| 2013 | I2E-VL | artificial | QTVVTQEPSLTVSPGGTVTITCGSSTGAVTSGNYPNWVQKKPGQAPRGLIGGTKFLAP |
| | | | GTPARFSGSLSGGKAALTLSGVQPEDEAEYYCVLWYSNRWVFGSGTKLTVL |
| 2014 | I2K-HCDR1 | artificial | KYAMN |
| 2015 | I2K-HCDR2 | artificial | RIRSKYNNYATYYAEAVKG |
| 2016 | I2K-HCDR3 | artificial | NENIGTSYISYWAY |
| 2017 | I2K-LCDR1 | artificial | GSSTGAVTSGNYPN |
| 2018 | I2K-LCDR2 | artificial | GTKFLAP |
| 2019 | I2K-LCDR3 | artificial | VLWYSNRWV |
| 2020 | I2K-VH | artificial | EVQLVESGGGLVQPGGSLKLSCAASGFTFNKYAMNWVRQAPGKGLEWVARIRSKYNNY |
| | | | ATYYAEAVKGRFTISRDDSKNTVYLQMNNLKTEDTAVYYCVRNENIGTSYISYWAYWG |
| | | | QGTLVTVSS |
| 2021 | I2K-VL | artificial | QTVVTQEPSLTVSPGGTVTITCGSSTGAVTSGNYPNWVQQKPGQAPRGLIGGTKFLAP |
| | | | GTPARFSGSLSGGKAALTLSGVQPEDEAEYYCVLWYSNRWVFGSGTKLTVL |
| 2022 | I2L-HCDR1 | artificial | KYAMN |
| 2023 | I2L-HCDR2 | artificial | RIRSKYNNYATYYADAVKD |
| 2024 | I2L-HCDR3 | artificial | AGNFGSSYISYFAY |
| 2025 | I2L-LCDR1 | artificial | GSSTGAVTSGNYPN |
| 2026 | I2L-LCDR2 | artificial | GTKFLAP |
| 2027 | I2L-LCDR3 | artificial | VLYYSNRWV |
| 2028 | I2L-VH | artificial | EVQLVESGGGLVQPGGSLKLSCAASGFTFNKYAMNWVRQAPGKGMEWVARIRSKYNNY |
| | | | ATYYADAVKDRFTISRDDSKNTLYLQMNNLKTEDTAVYYCVRAGNFGSSYISYFAYWG |
| | | | QGTLVTVSS |
| 2029 | I2L-VL | artificial | QTVVTQEPSLTVSPGGTVTITCGSSTGAVTSGNYPNWIQKKPGQAPRGLIGGTKFLAP |
| | | | GTPARFSGSLEGGKAALTLSGVQPEDEAEYYCVLYYSNRWVFGSGTKLTVL |
| 2030 | I2M-HCDR1 | artificial | KYAMN |
| 2031 | I2M-HCDR2 | artificial | RIRSKYNNYATYYADAVKD |
| 2032 | I2M-HCDR3 | artificial | AGNFGTSYISYWAY |
| 2033 | I2M-LCDR1 | artificial | GSSTGAVTSGNYPN |
| 2034 | I2M-LCDR2 | artificial | GTKFLAP |
| 2035 | I2M-LCDR3 | artificial | VLWYSNRWV |
| 2036 | I2M-VH | artificial | EVQLVESGGGLVQPGGSLKLSCAASGFTFNKYAMNWVRQAPGKGMEWVARIRSKYNNY |
| | | | ATYYADAVKDRFTISRDDSKNTLYLQMNNLKTEDTAVYYCVRAGNFGTSYISYWAYWG |
| | | | QGTLVTVSS |
| 2037 | I2M-VL | artificial | QTVVTQEPSLTVSPGGTVTITCGSSTGAVTSGNYPNWVQKKPGQAPRGLIGGTKFLAP |
| | | | GTPARFSGSLSGGKAALTLSGVQPEDEAEYYCVLWYSNRWVFGSGTKLTVL |
| 2038 | I2M2- | artificial | KYAIN |
| | HCDR1 | | |
| 2039 | I2M2- | artificial | RIRSKYNNYATYYADAVKD |
| | HCDR2 | | |
| 2040 | I2M2- | artificial | NANFGTSYISYFAY |
| | HCDR3 | | |
| 2041 | I2M2- | artificial | GSSTGAVTSGNYPN |
| | LCDR1 | | |
| 2042 | I2M2- | artificial | GTKFLAP |
| | LCDR2 | | |
| 2043 | I2M2- | artificial | VLWYSNRWV |
| | LCDR3 | | |
| 2044 | I2M2-VH | artificial | EVQLVESGGGLVQPGGSLKLSCAASGFTFNKYAINWVREAPGKGLEWVARIRSKYNNY |
| | | | ATYYADAVKDRFTISRDDSKNTAYLQMNNLKTEDTAVYYCVRNANFGTSYISYFAYWG |
| | | | QGTLVTVSS |
| 2045 | I2M2-VL | artificial | QTVVTQEPSLTVSPGGTVTLTCGSSTGAVTSGNYPNWVQKKPGQAPRGLIGGTKFLAP |
| | | | GTPARFSGSLLGGKAALTLSGVQPEDEAEYYCVLWYSNRWVFGSGTKLTVL |
| 2046 | consensus_ | artificial | KYAMN |
| | sequence_1- | | |
| | HCDR1 | | |
| 2047 | consensus_ | artificial | RIRSKYNNYATYYADSVKD |
| | sequence_1- | | |
| | HCDR2 | | |
| 2048 | consensus_ | artificial | HGNFGNSYJSXWAY |
| | sequence_1- | | |
| | HCDR3 | | |
| 2049 | consensus_ | artificial | GSSTGAVTSGYYPN |
| | sequence_1- | | |
| | LCDR1 | | |
| 2050 | consensus_ | artificial | GTKFLAP |
| | sequence_1- | | |
| | LCDR2 | | |
| 2051 | consensus_ | artificial | ALWYSNRWV |
| | sequence_1- | | |
| | LCDR3 | | |
| 2052 | consensus_ | artificial | EVQLVESGGGLVQPGGSLKLSCAASGFTFNKYAMNWVRQAPGKGLEWVARIRSKYNNY |
| | sequence_1- | | ATYYADSVKDRFTISRDDSKNTAYLQMNNLKTEDTAVYYCVRHGNFGNSYJSXWAYWG |
| | VH | | QGTLVTVSS |
| 2053 | consensus_ | artificial | QTVVTQEPSLTVSPGGTVTLTCGSSTGAVTSGYYPNWVQQKPGQAPRGLIGGTKFLAP |
| | sequence_1- | | GTPARFSGSLLGGKAALTLSGVQPEDEAEYYCALWYSNRWVFGGGTKLTVL |
| | VL | | |
| 2054 | consensus_ | artificial | KYAMN |
| | sequence_2 | | |
| | HCDR1 | | |
| 2055 | consensus_ | artificial | RIRSKYNNYATYYADSVKX |
| | sequence_2 | | |
| | HCDR2 | | |
| 2056 | consensus_ | artificial | HGNFGNSYJSYWAY |
| | sequence_2 | | |
| | HCDR3 | | |
| 2057 | consensus_ | artificial | GSSTGAVTSGYYPN |
| | sequence_2 | | |
| | LCDR1 | | |
| 2058 | consensus_ | artificial | GTKFLAP |
| | sequence_2 | | |
| | LCDR2 | | |
| 2059 | consensus_ | artificial | ALWYSNRWV |
| | sequence_2 | | |
| | LCDR3 | | |
| 2060 | consensus_ | artificial | EVQLVESGGGLVQPGGSLKLSCAASGFTFNKYAMNWVRQAPGKGLEWVARIRSKYNNY |
| | sequence_2 | | ATYYADSVKXRFTISRDDSKNTAYLQMNNLKTEDTAVYYCVRHGNFGNSYJSYWAYWG |
| | VH | | QGTLVTVSS |
| 2061 | consensus_ | artificial | QTVVTQEPSLTVSPGGTVTLTCGSSTGAVTSGYYPNWVQQKPGQAPRGLIGGTKFLAP |
| | sequence_2 | | GTPARFSGSLLGGKAALTLSGVQPEDEAEYYCALWYSNRWVFGGGTKLTVL |
| | VL | | |
| 2062 | consensus_ | artificial | KYAMN |
| | sequence_3- | | |
| | HCDR1 | | |
| 2063 | consensus_ | artificial | RIRSKYNNYATYYADSVKX |
| | sequence_3- | | |
| | HCDR2 | | |
| 2064 | consensus_ | artificial | HGNFGNSYISYWAY |
| | sequence_3- | | |
| | HCDR3 | | |
| 2065 | consensus_ | artificial | GSSTGAVTSGYYPN |
| | sequence_3- | | |
| | LCDR1 | | |
| 2066 | consensus_ | artificial | GTKFLAP |
| | sequence_3- | | |
| | LCDR2 | | |
| 2067 | consensus_ | artificial | ALWYSNRWV |
| | sequence_3- | | |
| | LCDR3 | | |
| 2068 | consensus_ | artificial | EVQLVESGGGLVQPGGSLKLSCAASGFTFNKYAMNWVRQAPGKGLEWVARIRSKYNNY |
| | sequence_3- | | ATYYADSVKXRFTISRDDSKNTAYLQMNNLKTEDTAVYYCVRHGNFGNSYISYWAYWG |
| | VH | | QGTLVTVSS |
| 2069 | consensus_ | artificial | QTVVTQEPSLTVSPGGTVTLTCGSSTGAVTSGYYPNWVQQKPGQAPRGLIGGTKFLAP |
| | sequence_3- | | GTPARFSGSLLGGKAALTLSGVQPEDEAEYYCALWYSNRWVFGGGTKLTVL |
| | VL | | |
| 2070 | consensus_ | artificial | EVQLVESGGGLVQPGGSLKLSCAASGFTFNKYAMNWVRQAPGKGLEWVARIRSKYNNY |
| | sequence_4- | | ATYYADSVKDRFTISRDDSKNTAYLQMNNLKTEDTAVYYCVRHGNFGNSYISYWAYWG |
| | VH | | QGTLVTVSS |
| 2071 | consensus_ | artificial | QTVVTQEPSLTVSPGGTVTLTCGSSTGAVTSGNYPNWVQQKPGQAPRGLIGGTKFLAP |
| | sequence_4- | | GTPARFSGSLLGGKAALTLSGVQPEDEAEYYCVLWYSNRWVFGGGTKLTVL |
| | VL | | |
| 2072 | BC A7 27- | artificial | NHIIH |
| | C4-G7- | | |
| | HCDR1 | | |
| 2073 | BC A7 27- | artificial | YINPYPGYHAYNEKFQG |
| | C4-G7- | | |
| | HCDR2 | | |
| 2074 | BC A7 27- | artificial | DGYYRDTDVLDY |
| | C4-G7- | | |
| | HCDR3 | | |
| 2075 | BC A7 27- | artificial | QASQDISNYLN |
| | C4-G7- | | |
| | LCDR1 | | |
| 2076 | BC A7 27- | artificial | YTSRLHT |
| | C4-G7- | | |
| | LCDR2 | | |
| 2077 | BC A7 27- | artificial | QQGNTLPWT |
| | C4-G7- | | |
| | LCDR3 | | |
| 2078 | BC A7 27- | artificial | QVQLVQSGAEVKKPGASVKVSCKASGYTFTNHIIHWVRQAPGQGLEWMGYINPYPGYH |
| | C4-G7-VH | | AYNEKFQGRATMTSDTSTSTVYMELSSLRSEDTAVYYCARDGYYRDTDVLDYWGQGTL |
| | | | VTVSS |
| 2079 | BC A7 27- | artificial | DIQMTQSPSSLSASVGDRVTITCQASQDISNYLNWYQQKPGKAPKLLIYYTSRLHTGV |
| | C4-G7-VL | | PSRFSGSGSGTDFTFTISSLEPEDIATYYCQQGNTLPWTFGQGTKVEIK |
| 2080 | BC A7 27- | artificial | QVQLVQSGAEVKKPGASVKVSCKASGYTFTNHIIHWVRQAPGQCLEWMGYINPYPGYH |
| | C4-G7 CC- | | AYNEKFQGRATMTSDTSTSTVYMELSSLRSEDTAVYYCARDGYYRDTDVLDYWGQGTL |
| | VH | | VTVSS |
| 2081 | BC A7 27- | artificial | DIQMTQSPSSLSASVGDRVTITCQASQDISNYLNWYQQKPGKAPKLLIYYTSRLHTGV |
| | C4-G7 CC- | | PSRFSGSGSGTDFTFTISSLEPEDIATYYCQQGNTLPWTFGCGTKVEIK |
| | VL | | |
| 2082 | BC A7 27- | artificial | EIQMTQSPSSLSASVGDRVTITCQASQDISNYLNWYQQKPGKAPKLLIYYTSRLHTGV |
| | C4-G7 CC EI | | PSRFSGSGSGTDFTFTISSLEPEDIATYYCQQGNTLPWTFGCGTKVEIK |
| | GQ-VL | | |
| 2083 | BC A7 27- | artificial | EIQMTQSPSSLSASVGDRVTITCQASQDISNYLNWYQQKPGKAPKLLIYYTSRLHTGV |
| | C4-G7 EI GQ | | PSRFSGSGSGTDFTFTISSLEPEDIATYYCQQGNTLPWTFGQGTKVEIK |
| | -VL | | |
| 2084 | BC A7 27- | artificial | QVQLVQSGAEVKKPGASVKVSCKASGYTFTNHIIHWVRQAPGQGLEWMGYINPYPGYH |
| | C4-G7X I2C | | AYNEKFQGRATMTSDTSTSTVYMELSSLRSEDTAVYYCARDGYYRDTDVLDYWGQGTL |
| | -Full | | VTVSSGGGGSGGGGSGGGGSDIQMTQSPSSLSASVGDRVTITCQASQDISNYLNWYQQ |
| | Sequence | | KPGKAPKLLIYYTSRLHTGVPSRFSGSGSGTDFTFTISSLEPEDIATYYCQQGNTLPW |
| | | | TFGQGTKVEIKSGGGGSEVQLVESGGGLVQPGGSLKLSCAASGFTFNKYAMNWVRQAP |
| | | | GKGLEWVARIRSKYNNYATYYADSVKDRFTISRDDSKNTAYLQMNNLKTEDTAVYYCV |
| | | | RHGNFGNSYISYWAYWGQGTLVTVSSGGGGSGGGGSGGGGSQTVVTQEPSLTVSPGGT |
| | | | VTLTCGSSTGAVTSGNYPNWVQQKPGQAPRGLIGGTKFLAPGTPARFSGSLLGGKAAL |
| | | | TLSGVQPEDEAEYYCVLWYSNRWVFGGGTKLTVL |
| 2085 | BC A7 27- | artificial | QVQLVQSGAEVKKPGASVKVSCKASGYTFTNHIIHWVRQAPGQGLEWMGYINPYPGYH |
| | C4-G7X I2E | | AYNEKFQGRATMTSDTSTSTVYMELSSLRSEDTAVYYCARDGYYRDTDVLDYWGQGTL |
| | -Full | | VTVSSGGGGSGGGGSGGGGSDIQMTQSPSSLSASVGDRVTITCQASQDISNYLNWYQQ |
| | Sequence | | KPGKAPKLLIYYTSRLHTGVPSRFSGSGSGTDFTFTISSLEPEDIATYYCQQGNTLPW |
| | | | TFGQGTKVEIKSGGGGSEVQLVESGGGLVQPGGSLKLSCAASGFTFNKYAINWVRQAP |
| | | | GKGLEWVARIRSKYNNYATYYADAVKDRFTISRDDSKNTVYLQMNNLKTEDTAVYYCA |
| | | | RAGNFGSSYISYWAYWGQGTLVTVSSGGGGSGGGGSGGGGSQTVVTQEPSLTVSPGGT |
| | | | VTITCGSSTGAVTSGNYPNWVQKKPGQAPRGLIGGTKFLAPGTPARFSGSLSGGKAAL |
| | | | TLSGVQPEDEAEYYCVLWYSNRWVFGSGTKLTVL |
| 2086 | BC A7 27- | artificial | QVQLVQSGAEVKKPGASVKVSCKASGYTFTNHIIHWVRQAPGQGLEWMGYINPYPGYH |
| | C4-G7X I2K | | AYNEKFQGRATMTSDTSTSTVYMELSSLRSEDTAVYYCARDGYYRDTDVLDYWGQGTL |
| | -Full | | VTVSSGGGGSGGGGSGGGGSDIQMTQSPSSLSASVGDRVTITCQASQDISNYLNWYQQ |
| | Sequence | | KPGKAPKLLIYYTSRLHTGVPSRFSGSGSGTDFTFTISSLEPEDIATYYCQQGNTLPW |
| | | | TFGQGTKVEIKSGGGGSEVQLVESGGGLVQPGGSLKLSCAASGFTENKYAMNWVRQAP |
| | | | GKGLEWVARIRSKYNNYATYYAEAVKGRFTISRDDSKNTVYLQMNNLKTEDTAVYYCV |
| | | | RNENIGTSYISYWAYWGQGTLVTVSSGGGGSGGGGSGGGGSQTVVTQEPSLTVSPGGT |
| | | | VTITCGSSTGAVTSGNYPNWVQQKPGQAPRGLIGGTKFLAPGTPARFSGSLSGGKAAL |
| | | | TLSGVQPEDEAEYYCVLWYSNRWVFGSGTKLTVL |
| 2087 | BC A7 27- | artificial | QVQLVQSGAEVKKPGASVKVSCKASGYTFTNHIIHWVRQAPGQGLEWMGYINPYPGYH |
| | C4-G7X I2L | | AYNEKFQGRATMTSDTSTSTVYMELSSLRSEDTAVYYCARDGYYRDTDVLDYWGQGTL |
| | -Full | | VTVSSGGGGSGGGGSGGGGSDIQMTQSPSSLSASVGDRVTITCQASQDISNYLNWYQQ |
| | Sequence | | KPGKAPKLLIYYTSRLHTGVPSRFSGSGSGTDFTFTISSLEPEDIATYYCQQGNTLPW |
| | | | TFGQGTKVEIKSGGGGSEVQLVESGGGLVQPGGSLKLSCAASGFTENKYAMNWVRQAP |
| | | | GKGMEWVARIRSKYNNYATYYADAVKDRFTISRDDSKNTLYLQMNNLKTEDTAVYYCV |
| | | | RAGNFGSSYISYFAYWGQGTLVTVSSGGGGSGGGGSGGGGSQTVVTQEPSLTVSPGGT |
| | | | VTITCGSSTGAVTSGNYPNWIQKKPGQAPRGLIGGTKFLAPGTPARFSGSLEGGKAAL |
| | | | TLSGVQPEDEAEYYCVLYYSNRWVFGSGTKLTVL |
| 2088 | BC A7 27- | artificial | QVQLVQSGAEVKKPGASVKVSCKASGYTFTNHIIHWVRQAPGQGLEWMGYINPYPGYH |
| | C4-G7X I2M | | AYNEKFQGRATMTSDTSTSTVYMELSSLRSEDTAVYYCARDGYYRDTDVLDYWGQGTL |
| | -Full | | VTVSSGGGGSGGGGSGGGGSDIQMTQSPSSLSASVGDRVTITCQASQDISNYLNWYQQ |
| | Sequence | | KPGKAPKLLIYYTSRLHTGVPSRFSGSGSGTDFTFTISSLEPEDIATYYCQQGNTLPW |
| | | | TFGQGTKVEIKSGGGGSEVQLVESGGGLVQPGGSLKLSCAASGFTENKYAMNWVRQAP |
| | | | GKGMEWVARIRSKYNNYATYYADAVKDRFTISRDDSKNTLYLQMNNLKTEDTAVYYCV |
| | | | RAGNFGTSYISYWAYWGQGTLVTVSSGGGGSGGGGSGGGGSQTVVTQEPSLTVSPGGT |
| | | | VTITCGSSTGAVTSGNYPNWVQKKPGQAPRGLIGGTKFLAPGTPARFSGSLSGGKAAL |
| | | | TLSGVQPEDEAEYYCVLWYSNRWVFGSGTKLTVL |
| 2089 | BC A7 27- | artificial | QVQLVQSGAEVKKPGASVKVSCKASGYTFTNHIIHWVRQAPGQGLEWMGYINPYPGYH |
| | C4-G7X I2M2 | | AYNEKFQGRATMTSDTSTSTVYMELSSLRSEDTAVYYCARDGYYRDTDVLDYWGQGTL |
| | -Full | | VTVSSGGGGSGGGGSGGGGSDIQMTQSPSSLSASVGDRVTITCQASQDISNYLNWYQQ |
| | Sequence | | KPGKAPKLLIYYTSRLHTGVPSRFSGSGSGTDFTFTISSLEPEDIATYYCQQGNTLPW |
| | | | TFGQGTKVEIKSGGGGSEVQLVESGGGLVQPGGSLKLSCAASGFTENKYAINWVREAP |
| | | | GKGLEWVARIRSKYNNYATYYADAVKDRFTISRDDSKNTAYLQMNNLKTEDTAVYYCV |
| | | | RNANFGTSYISYFAYWGQGTLVTVSSGGGGSGGGGSGGGGSQTVVTQEPSLTVSPGGT |
| | | | VTLTCGSSTGAVTSGNYPNWVQKKPGQAPRGLIGGTKFLAPGTPARFSGSLLGGKAAL |
| | | | TLSGVQPEDEAEYYCVLWYSNRWVFGSGTKLTVL |
| 2090 | BC A7 27- | artificial | QVQLVQSGAEVKKPGASVKVSCKASGYTFTNHIIHWVRQAPGQCLEWMGYINPYPGYH |
| | C4-G7 CC X | | AYNEKFQGRATMTSDTSTSTVYMELSSLRSEDTAVYYCARDGYYRDTDVLDYWGQGTL |
| | I2C x scFc | | VTVSSGGGGSGGGGSGGGGSDIQMTQSPSSLSASVGDRVTITCQASQDISNYLNWYQQ |
| | -Full | | KPGKAPKLLIYYTSRLHTGVPSRFSGSGSGTDFTFTISSLEPEDIATYYCQQGNTLPW |
| | Sequence | | TFGCGTKVEIKSGGGGSEVQLVESGGGLVQPGGSLKLSCAASGFTFNKYAMNWVRQAP |
| | | | GKGLEWVARIRSKYNNYATYYADSVKDRFTISRDDSKNTAYLQMNNLKTEDTAVYYCV |
| | | | RHGNFGNSYISYWAYWGQGTLVTVSSGGGGSGGGGSGGGGSQTVVTQEPSLTVSPGGT |
| | | | VTLTCGSSTGAVTSGNYPNWVQQKPGQAPRGLIGGTKFLAPGTPARFSGSLLGGKAAL |
| | | | TLSGVQPEDEAEYYCVLWYSNRWVFGGGTKLTVLGGGGDKTHTCPPCPAPELLGGPSV |
| | | | FLFPPKPKDTLMISRTPEVTCVVVDVSHEDPEVKENWYVDGVEVHNAKTKPCEEQYGS |
| | | | TYRCVSVLTVLHQDWLNGKEYKCKVSNKALPAPIEKTISKAKGQPREPQVYTLPPSRE |
| | | | EMTKNQVSLTCLVKGFYPSDIAVEWESNGQPENNYKTTPPVLDSDGSFFLYSKLTVDK |
| | | | SRWQQGNVFSCSVMHEALHNHYTQKSLSLSPGKGGGGSGGGGSGGGGSGGGGSGGGGS |
| | | | GGGGSDKTHTCPPCPAPELLGGPSVFLFPPKPKDTLMISRTPEVTCVVVDVSHEDPEV |
| | | | KFNWYVDGVEVHNAKTKPCEEQYGSTYRCVSVLTVLHQDWLNGKEYKCKVSNKALPAP |
| | | | IEKTISKAKGQPREPQVYTLPPSREEMTKNQVSLTCLVKGFYPSDIAVEWESNGQPEN |
| | | | NYKTTPPVLDSDGSFFLYSKLTVDKSRWQQGNVFSCSVMHEALHNHYTQKSLSLSPGK |
| 2091 | BC A7 27- | artificial | QVQLVQSGAEVKKPGASVKVSCKASGYTFTNHIIHWVRQAPGQCLEWMGYINPYPGYH |
| | C4-G7 CC X | | AYNEKFQGRATMTSDTSTSTVYMELSSLRSEDTAVYYCARDGYYRDTDVLDYWGQGTL |
| | I2E x scFc | | VTVSSGGGGSGGGGSGGGGSDIQMTQSPSSLSASVGDRVTITCQASQDISNYLNWYQQ |
| | -Full | | KPGKAPKLLIYYTSRLHTGVPSRFSGSGSGTDFTFTISSLEPEDIATYYCQQGNTLPW |
| | Sequence | | TFGCGTKVEIKSGGGGSEVQLVESGGGLVQPGGSLKLSCAASGFTFNKYAINWVRQAP |
| | | | GKGLEWVARIRSKYNNYATYYADAVKDRFTISRDDSKNTVYLQMNNLKTEDTAVYYCA |
| | | | RAGNFGSSYISYWAYWGQGTLVTVSSGGGGSGGGGSGGGGSQTVVTQEPSLTVSPGGT |
| | | | VTITCGSSTGAVTSGNYPNWVQKKPGQAPRGLIGGTKFLAPGTPARFSGSLSGGKAAL |
| | | | TLSGVQPEDEAEYYCVLWYSNRWVFGSGTKLTVLGGGGDKTHTCPPCPAPELLGGPSV |
| | | | FLFPPKPKDTLMISRTPEVTCVVVDVSHEDPEVKFNWYVDGVEVHNAKTKPCEEQYGS |
| | | | TYRCVSVLTVLHQDWLNGKEYKCKVSNKALPAPIEKTISKAKGQPREPQVYTLPPSRE |
| | | | EMTKNQVSLTCLVKGFYPSDIAVEWESNGQPENNYKTTPPVLDSDGSFFLYSKLTVDK |
| | | | SRWQQGNVFSCSVMHEALHNHYTQKSLSLSPGKGGGGSGGGGSGGGGSGGGGSGGGGS |
| | | | GGGGSDKTHTCPPCPAPELLGGPSVFLFPPKPKDTLMISRTPEVTCVVVDVSHEDPEV |
| | | | KFNWYVDGVEVHNAKTKPCEEQYGSTYRCVSVLTVLHQDWLNGKEYKCKVSNKALPAP |
| | | | IEKTISKAKGQPREPQVYTLPPSREEMTKNQVSLTCLVKGFYPSDIAVEWESNGQPEN |
| | | | NYKTTPPVLDSDGSFFLYSKLTVDKSRWQQGNVFSCSVMHEALHNHYTQKSLSLSPGK |
| 2092 | BC A7 27- | artificial | QVQLVQSGAEVKKPGASVKVSCKASGYTFTNHIIHWVRQAPGQCLEWMGYINPYPGYH |
| | C4-G7 CC X | | AYNEKFQGRATMTSDTSTSTVYMELSSLRSEDTAVYYCARDGYYRDTDVLDYWGQGTL |
| | I2K x scFc | | VTVSSGGGGSGGGGSGGGGSDIQMTQSPSSLSASVGDRVTITCQASQDISNYLNWYQQ |
| | Full | | KPGKAPKLLIYYTSRLHTGVPSRFSGSGSGTDFTFTISSLEPEDIATYYCQQGNTLPW |
| | Sequence | | TFGCGTKVEIKSGGGGSEVQLVESGGGLVQPGGSLKLSCAASGFTENKYAMNWVRQAP |
| | | | GKGLEWVARIRSKYNNYATYYAEAVKGRFTISRDDSKNTVYLQMNNLKTEDTAVYYCV |
| | | | RNENIGTSYISYWAYWGQGTLVTVSSGGGGSGGGGSGGGGSQTVVTQEPSLTVSPGGT |
| | | | VTITCGSSTGAVTSGNYPNWVQQKPGQAPRGLIGGTKFLAPGTPARFSGSLSGGKAAL |
| | | | TLSGVQPEDEAEYYCVLWYSNRWVFGSGTKLTVLGGGGDKTHTCPPCPAPELLGGPSV |
| | | | FLFPPKPKDTLMISRTPEVTCVVVDVSHEDPEVKFNWYVDGVEVHNAKTKPCEEQYGS |
| | | | TYRCVSVLTVLHQDWLNGKEYKCKVSNKALPAPIEKTISKAKGQPREPQVYTLPPSRE |
| | | | EMTKNQVSLTCLVKGFYPSDIAVEWESNGQPENNYKTTPPVLDSDGSFFLYSKLTVDK |
| | | | SRWQQGNVFSCSVMHEALHNHYTQKSLSLSPGKGGGGSGGGGSGGGGSGGGGSGGGGS |
| | | | GGGGSDKTHTCPPCPAPELLGGPSVFLFPPKPKDTLMISRTPEVTCVVVDVSHEDPEV |
| | | | KFNWYVDGVEVHNAKTKPCEEQYGSTYRCVSVLTVLHQDWLNGKEYKCKVSNKALPAP |
| | | | IEKTISKAKGQPREPQVYTLPPSREEMTKNQVSLTCLVKGFYPSDIAVEWESNGQPEN |
| | | | NYKTTPPVLDSDGSFFLYSKLTVDKSRWQQGNVFSCSVMHEALHNHYTQKSLSLSPGK |
| 2093 | BC A7 27- | artificial | QVQLVQSGAEVKKPGASVKVSCKASGYTFTNHIIHWVRQAPGQCLEWMGYINPYPGYH |
| | C4-G7 CC X | | AYNEKFQGRATMTSDTSTSTVYMELSSLRSEDTAVYYCARDGYYRDTDVLDYWGQGTL |
| | I2L x scFc | | VTVSSGGGGSGGGGSGGGGSDIQMTQSPSSLSASVGDRVTITCQASQDISNYLNWYQQ |
| | -Full | | KPGKAPKLLIYYTSRLHTGVPSRFSGSGSGTDFTFTISSLEPEDIATYYCQQGNTLPW |
| | Sequence | | TFGCGTKVEIKSGGGGSEVQLVESGGGLVQPGGSLKLSCAASGFTFNKYAMNWVRQAP |
| | | | GKGMEWVARIRSKYNNYATYYADAVKDRFTISRDDSKNTLYLQMNNLKTEDTAVYYCV |
| | | | RAGNFGSSYISYFAYWGQGTLVTVSSGGGGSGGGGSGGGGSQTVVTQEPSLTVSPGGT |
| | | | VTITCGSSTGAVTSGNYPNWIQKKPGQAPRGLIGGTKFLAPGTPARFSGSLEGGKAAL |
| | | | TLSGVQPEDEAEYYCVLYYSNRWVFGSGTKLTVLGGGGDKTHTCPPCPAPELLGGPSV |
| | | | FLFPPKPKDTLMISRTPEVTCVVVDVSHEDPEVKENWYVDGVEVHNAKTKPCEEQYGS |
| | | | TYRCVSVLTVLHQDWLNGKEYKCKVSNKALPAPIEKTISKAKGQPREPQVYTLPPSRE |
| | | | EMTKNQVSLTCLVKGFYPSDIAVEWESNGQPENNYKTTPPVLDSDGSFFLYSKLTVDK |
| | | | SRWQQGNVFSCSVMHEALHNHYTQKSLSLSPGKGGGGSGGGGSGGGGSGGGGSGGGGS |
| | | | GGGGSDKTHTCPPCPAPELLGGPSVFLFPPKPKDTLMISRTPEVTCVVVDVSHEDPEV |
| | | | KFNWYVDGVEVHNAKTKPCEEQYGSTYRCVSVLTVLHQDWLNGKEYKCKVSNKALPAP |
| | | | IEKTISKAKGQPREPQVYTLPPSREEMTKNQVSLTCLVKGFYPSDIAVEWESNGQPEN |
| | | | NYKTTPPVLDSDGSFFLYSKLTVDKSRWQQGNVFSCSVMHEALHNHYTQKSLSLSPGK |
| 2094 | BC A7 27- | artificial | QVQLVQSGAEVKKPGASVKVSCKASGYTFTNHIIHWVRQAPGQCLEWMGYINPYPGYH |
| | C4-G7 CC X | | AYNEKFQGRATMTSDTSTSTVYMELSSLRSEDTAVYYCARDGYYRDTDVLDYWGQGTL |
| | I2M x scFc | | VTVSSGGGGSGGGGSGGGGSDIQMTQSPSSLSASVGDRVTITCQASQDISNYLNWYQQ |
| | -Full | | KPGKAPKLLIYYTSRLHTGVPSRFSGSGSGTDFTFTISSLEPEDIATYYCQQGNTLPW |
| | Sequence | | TFGCGTKVEIKSGGGGSEVQLVESGGGLVQPGGSLKLSCAASGFTENKYAMNWVRQAP |
| | | | GKGMEWVARIRSKYNNYATYYADAVKDRFTISRDDSKNTLYLQMNNLKTEDTAVYYCV |
| | | | RAGNFGTSYISYWAYWGQGTLVTVSSGGGGSGGGGSGGGGSQTVVTQEPSLTVSPGGT |
| | | | VTITCGSSTGAVTSGNYPNWVQKKPGQAPRGLIGGTKFLAPGTPARFSGSLSGGKAAL |
| | | | TLSGVQPEDEAEYYCVLWYSNRWVFGSGTKLTVLGGGGDKTHTCPPCPAPELLGGPSV |
| | | | FLFPPKPKDTLMISRTPEVTCVVVDVSHEDPEVKFNWYVDGVEVHNAKTKPCEEQYGS |
| | | | TYRCVSVLTVLHQDWLNGKEYKCKVSNKALPAPIEKTISKAKGQPREPQVYTLPPSRE |
| | | | EMTKNQVSLTCLVKGFYPSDIAVEWESNGQPENNYKTTPPVLDSDGSFFLYSKLTVDK |
| | | | SRWQQGNVFSCSVMHEALHNHYTQKSLSLSPGKGGGGSGGGGSGGGGSGGGGSGGGGS |
| | | | GGGGSDKTHTCPPCPAPELLGGPSVFLFPPKPKDTLMISRTPEVTCVVVDVSHEDPEV |
| | | | KFNWYVDGVEVHNAKTKPCEEQYGSTYRCVSVLTVLHQDWLNGKEYKCKVSNKALPAP |
| | | | IEKTISKAKGQPREPQVYTLPPSREEMTKNQVSLTCLVKGFYPSDIAVEWESNGQPEN |
| | | | NYKTTPPVLDSDGSFFLYSKLTVDKSRWQQGNVFSCSVMHEALHNHYTQKSLSLSPGK |
| 2095 | BC A7 27- | artificial | QVQLVQSGAEVKKPGASVKVSCKASGYTFTNHIIHWVRQAPGQCLEWMGYINPYPGYH |
| | C4-G7 CC X | | AYNEKFQGRATMTSDTSTSTVYMELSSLRSEDTAVYYCARDGYYRDTDVLDYWGQGTL |
| | I2M2 x scFc | | VTVSSGGGGSGGGGSGGGGSDIQMTQSPSSLSASVGDRVTITCQASQDISNYLNWYQQ |
| | -Full | | KPGKAPKLLIYYTSRLHTGVPSRFSGSGSGTDFTFTISSLEPEDIATYYCQQGNTLPW |
| | Sequence | | TFGCGTKVEIKSGGGGSEVQLVESGGGLVQPGGSLKLSCAASGFTFNKYAINWVREAP |
| | | | GKGLEWVARIRSKYNNYATYYADAVKDRFTISRDDSKNTAYLQMNNLKTEDTAVYYCV |
| | | | RNANFGTSYISYFAYWGQGTLVTVSSGGGGSGGGGSGGGGSQTVVTQEPSLTVSPGGT |
| | | | VTLTCGSSTGAVTSGNYPNWVQKKPGQAPRGLIGGTKFLAPGTPARFSGSLLGGKAAL |
| | | | TLSGVQPEDEAEYYCVLWYSNRWVFGSGTKLTVLGGGGDKTHTCPPCPAPELLGGPSV |
| | | | FLFPPKPKDTLMISRTPEVTCVVVDVSHEDPEVKFNWYVDGVEVHNAKTKPCEEQYGS |
| | | | TYRCVSVLTVLHQDWLNGKEYKCKVSNKALPAPIEKTISKAKGQPREPQVYTLPPSRE |
| | | | EMTKNQVSLTCLVKGFYPSDIAVEWESNGQPENNYKTTPPVLDSDGSFFLYSKLTVDK |
| | | | SRWQQGNVFSCSVMHEALHNHYTQKSLSLSPGKGGGGSGGGGSGGGGSGGGGSGGGGS |
| | | | GGGGSDKTHTCPPCPAPELLGGPSVFLFPPKPKDTLMISRTPEVTCVVVDVSHEDPEV |
| | | | KFNWYVDGVEVHNAKTKPCEEQYGSTYRCVSVLTVLHQDWLNGKEYKCKVSNKALPAP |
| | | | IEKTISKAKGQPREPQVYTLPPSREEMTKNQVSLTCLVKGFYPSDIAVEWESNGQPEN |
| | | | NYKTTPPVLDSDGSFFLYSKLTVDKSRWQQGNVFSCSVMHEALHNHYTQKSLSLSPGK |
| 2096 | CD123 24- | artificial | HYAMS |
| | B4-f NK CC | | |
| | -HCDR1 | | |
| 2097 | CD123 24- | artificial | AVSGGGDKTLYADAVKG |
| | B4-f NK CC | | |
| | -HCDR2 | | |
| 2098 | CD123 24- | artificial | LRGFYYGMDV |
| | B4-f NK CC | | |
| | -HCDR3 | | |
| 2099 | CD123 24- | artificial | RSSQSLLHSNKYNYLD |
| | B4-f NK CC | | |
| | -LCDR1 | | |
| 2100 | CD123 24- | artificial | LGSNRAS |
| | B4-f NK CC | | |
| | -LCDR2 | | |
| 2101 | CD123 24- | artificial | MQALQTPPIT |
| | B4-f NK CC | | |
| | -LCDR3 | | |
| 2102 | CD123 24- | artificial | EVQLLESGGGLVQPGGSLRLSCAASGFTFSHYAMSWVRQAPGKCLEWVSAVSGGGDKT |
| | B4-f NK CC | | LYADAVKGRFTISRDNSKNTLFLQMNSLRAEDTAIYYCARLRGFYYGMDVWGQGTTVT |
| | -VH | | VSS |
| 2103 | CD123 24- | artificial | DIVLTQSPLSLPVTPGEPASISCRSSQSLLHSNKYNYLDWYLQKPGQSPQLLIYLGSN |
| | B4-f NK CC | | RASGVPDRFSGSGSGTDFTLKISRVEAEDVGVYYCMQALQTPPITFGCGTRLEIK |
| | -VL | | |
| 2104 | CD123 24- | artificial | EIVLTQSPLSLPVTPGEPASISCRSSQSLLHSNKYNYLDWYLQKPGQSPQLLIYLGSN |
| | B4-f NK CC | | RASGVPDRFSGSGSGTDFTLKISRVEAEDVGVYYCMQALQTPPITFGCGTRLEIK |
| | EI GQ-VL | | |
| 2105 | CD123 24- | artificial | EVQLLESGGGLVQPGGSLRLSCAASGFTFSHYAMSWVRQAPGKCLEWVSAVSGGGDKT |
| | B4-f NK CC | | LYADAVKGRFTISRDNSKNTLFLQMNSLRAEDTAIYYCARLRGFYYGMDVWGQGTTVT |
| | x I2C x | | VSSGGGGSGGGGSGGGGSDIVLTQSPLSLPVTPGEPASISCRSSQSLLHSNKYNYLDW |
| | ScFc-Full | | YLQKPGQSPQLLIYLGSNRASGVPDRFSGSGSGTDFTLKISRVEAEDVGVYYCMQALQ |
| | Sequence | | TPPITFGCGTRLEIKSGGGGSEVQLVESGGGLVQPGGSLKLSCAASGFTENKYAMNWV |
| | | | RQAPGKGLEWVARIRSKYNNYATYYADSVKDRFTISRDDSKNTAYLQMNNLKTEDTAV |
| | | | YYCVRHGNFGNSYISYWAYWGQGTLVTVSSGGGGSGGGGSGGGGSQTVVTQEPSLTVS |
| | | | PGGTVTLTCGSSTGAVTSGNYPNWVQQKPGQAPRGLIGGTKFLAPGTPARFSGSLLGG |
| | | | KAALTLSGVQPEDEAEYYCVLWYSNRWVFGGGTKLTVLGGGGDKTHTCPPCPAPELLG |
| | | | GPSVFLFPPKPKDTLMISRTPEVTCVVVDVSHEDPEVKFNWYVDGVEVHNAKTKPCEE |
| | | | QYGSTYRCVSVLTVLHQDWLNGKEYKCKVSNKALPAPIEKTISKAKGQPREPQVYTLP |
| | | | PSREEMTKNQVSLTCLVKGFYPSDIAVEWESNGQPENNYKTTPPVLDSDGSFFLYSKL |
| | | | TVDKSRWQQGNVFSCSVMHEALHNHYTQKSLSLSPGKGGGGSGGGGSGGGGSGGGGSG |
| | | | GGGSGGGGSDKTHTCPPCPAPELLGGPSVFLFPPKPKDTLMISRTPEVTCVVVDVSHE |
| | | | DPEVKFNWYVDGVEVHNAKTKPCEEQYGSTYRCVSVLTVLHQDWLNGKEYKCKVSNKA |
| | | | LPAPIEKTISKAKGQPREPQVYTLPPSREEMTKNQVSLTCLVKGFYPSDIAVEWESNG |
| | | | QPENNYKTTPPVLDSDGSFFLYSKLTVDKSRWQQGNVFSCSVMHEALHNHYTQKSLSL |
| | | | SPGK |
| 2106 | CD123 24- | artificial | EVQLLESGGGLVQPGGSLRLSCAASGFTFSHYAMSWVRQAPGKCLEWVSAVSGGGDKT |
| | B4-f NK CC | | LYADAVKGRFTISRDNSKNTLFLQMNSLRAEDTAIYYCARLRGFYYGMDVWGQGTTVT |
| | x I2E x | | VSSGGGGSGGGGSGGGGSDIVLTQSPLSLPVTPGEPASISCRSSQSLLHSNKYNYLDW |
| | scFc-Full | | YLQKPGQSPQLLIYLGSNRASGVPDRFSGSGSGTDFTLKISRVEAEDVGVYYCMQALQ |
| | Sequence | | TPPITFGCGTRLEIKSGGGGSEVQLVESGGGLVQPGGSLKLSCAASGFTENKYAINWV |
| | | | RQAPGKGLEWVARIRSKYNNYATYYADAVKDRFTISRDDSKNTVYLQMNNLKTEDTAV |
| | | | YYCARAGNFGSSYISYWAYWGQGTLVTVSSGGGGSGGGGSGGGGSQTVVTQEPSLTVS |
| | | | PGGTVTITCGSSTGAVTSGNYPNWVQKKPGQAPRGLIGGTKFLAPGTPARFSGSLSGG |
| | | | KAALTLSGVQPEDEAEYYCVLWYSNRWVFGSGTKLTVLGGGGDKTHTCPPCPAPELLG |
| | | | GPSVFLFPPKPKDTLMISRTPEVTCVVVDVSHEDPEVKFNWYVDGVEVHNAKTKPCEE |
| | | | QYGSTYRCVSVLTVLHQDWLNGKEYKCKVSNKALPAPIEKTISKAKGQPREPQVYTLP |
| | | | PSREEMTKNQVSLTCLVKGFYPSDIAVEWESNGQPENNYKTTPPVLDSDGSFFLYSKL |
| | | | TVDKSRWQQGNVFSCSVMHEALHNHYTQKSLSLSPGKGGGGSGGGGSGGGGSGGGGSG |
| | | | GGGSGGGGSDKTHTCPPCPAPELLGGPSVFLFPPKPKDTLMISRTPEVTCVVVDVSHE |
| | | | DPEVKFNWYVDGVEVHNAKTKPCEEQYGSTYRCVSVLTVLHQDWLNGKEYKCKVSNKA |
| | | | LPAPIEKTISKAKGQPREPQVYTLPPSREEMTKNQVSLTCLVKGFYPSDIAVEWESNG |
| | | | QPENNYKTTPPVLDSDGSFFLYSKLTVDKSRWQQGNVFSCSVMHEALHNHYTQKSLSL |
| | | | SPGK |
| 2107 | CD123 24- | artificial | EVQLLESGGGLVQPGGSLRLSCAASGFTFSHYAMSWVRQAPGKCLEWVSAVSGGGDKT |
| | B4-f NK CC | | LYADAVKGRFTISRDNSKNTLFLQMNSLRAEDTAIYYCARLRGFYYGMDVWGQGTTVT |
| | x I2K x | | VSSGGGGSGGGGSGGGGSDIVLTQSPLSLPVTPGEPASISCRSSQSLLHSNKYNYLDW |
| | scFc-Full | | YLQKPGQSPQLLIYLGSNRASGVPDRFSGSGSGTDFTLKISRVEAEDVGVYYCMQALQ |
| | Sequence | | TPPITFGCGTRLEIKSGGGGSEVQLVESGGGLVQPGGSLKLSCAASGFTENKYAMNWV |
| | | | RQAPGKGLEWVARIRSKYNNYATYYAEAVKGRFTISRDDSKNTVYLQMNNLKTEDTAV |
| | | | YYCVRNENIGTSYISYWAYWGQGTLVTVSSGGGGSGGGGSGGGGSQTVVTQEPSLTVS |
| | | | PGGTVTITCGSSTGAVTSGNYPNWVQQKPGQAPRGLIGGTKFLAPGTPARFSGSLSGG |
| | | | KAALTLSGVQPEDEAEYYCVLWYSNRWVFGSGTKLTVLGGGGDKTHTCPPCPAPELLG |
| | | | GPSVFLFPPKPKDTLMISRTPEVTCVVVDVSHEDPEVKFNWYVDGVEVHNAKTKPCEE |
| | | | QYGSTYRCVSVLTVLHQDWLNGKEYKCKVSNKALPAPIEKTISKAKGQPREPQVYTLP |
| | | | PSREEMTKNQVSLTCLVKGFYPSDIAVEWESNGQPENNYKTTPPVLDSDGSFFLYSKL |
| | | | TVDKSRWQQGNVFSCSVMHEALHNHYTQKSLSLSPGKGGGGSGGGGSGGGGSGGGGSG |
| | | | GGGSGGGGSDKTHTCPPCPAPELLGGPSVFLFPPKPKDTLMISRTPEVTCVVVDVSHE |
| | | | DPEVKFNWYVDGVEVHNAKTKPCEEQYGSTYRCVSVLTVLHQDWLNGKEYKCKVSNKA |
| | | | LPAPIEKTISKAKGQPREPQVYTLPPSREEMTKNQVSLTCLVKGFYPSDIAVEWESNG |
| | | | QPENNYKTTPPVLDSDGSFFLYSKLTVDKSRWQQGNVFSCSVMHEALHNHYTQKSLSL |
| | | | SPGK |
| 2108 | CD123 24- | artificial | EVQLLESGGGLVQPGGSLRLSCAASGFTFSHYAMSWVRQAPGKCLEWVSAVSGGGDKT |
| | B4-f NK CC | | LYADAVKGRFTISRDNSKNTLFLQMNSLRAEDTAIYYCARLRGFYYGMDVWGQGTTVT |
| | x I2L x | | VSSGGGGSGGGGSGGGGSDIVLTQSPLSLPVTPGEPASISCRSSQSLLHSNKYNYLDW |
| | scFc-Full | | YLQKPGQSPQLLIYLGSNRASGVPDRFSGSGSGTDFTLKISRVEAEDVGVYYCMQALQ |
| | Sequence | | TPPITFGCGTRLEIKSGGGGSEVQLVESGGGLVQPGGSLKLSCAASGFTENKYAMNWV |
| | | | RQAPGKGMEWVARIRSKYNNYATYYADAVKDRFTISRDDSKNTLYLQMNNLKTEDTAV |
| | | | YYCVRAGNFGSSYISYFAYWGQGTLVTVSSGGGGSGGGGSGGGGSQTVVTQEPSLTVS |
| | | | PGGTVTITCGSSTGAVTSGNYPNWIQKKPGQAPRGLIGGTKFLAPGTPARFSGSLEGG |
| | | | KAALTLSGVQPEDEAEYYCVLYYSNRWVFGSGTKLTVLGGGGDKTHTCPPCPAPELLG |
| | | | GPSVFLFPPKPKDTLMISRTPEVTCVVVDVSHEDPEVKENWYVDGVEVHNAKTKPCEE |
| | | | QYGSTYRCVSVLTVLHQDWLNGKEYKCKVSNKALPAPIEKTISKAKGQPREPQVYTLP |
| | | | PSREEMTKNQVSLTCLVKGFYPSDIAVEWESNGQPENNYKTTPPVLDSDGSFFLYSKL |
| | | | TVDKSRWQQGNVFSCSVMHEALHNHYTQKSLSLSPGKGGGGSGGGGSGGGGSGGGGSG |
| | | | GGGSGGGGSDKTHTCPPCPAPELLGGPSVFLFPPKPKDTLMISRTPEVTCVVVDVSHE |
| | | | DPEVKFNWYVDGVEVHNAKTKPCEEQYGSTYRCVSVLTVLHQDWLNGKEYKCKVSNKA |
| | | | LPAPIEKTISKAKGQPREPQVYTLPPSREEMTKNQVSLTCLVKGFYPSDIAVEWESNG |
| | | | QPENNYKTTPPVLDSDGSFFLYSKLTVDKSRWQQGNVFSCSVMHEALHNHYTQKSLSL |
| | | | SPGK |
| 2109 | CD123 24- | artificial | EVQLLESGGGLVQPGGSLRLSCAASGFTFSHYAMSWVRQAPGKCLEWVSAVSGGGDKT |
| | B4-f NK CC | | LYADAVKGRFTISRDNSKNTLFLQMNSLRAEDTAIYYCARLRGFYYGMDVWGQGTTVT |
| | x I2M X | | VSSGGGGSGGGGSGGGGSDIVLTQSPLSLPVTPGEPASISCRSSQSLLHSNKYNYLDW |
| | scFc-Full | | YLQKPGQSPQLLIYLGSNRASGVPDRFSGSGSGTDFTLKISRVEAEDVGVYYCMQALQ |
| | Sequence | | TPPITFGCGTRLEIKSGGGGSEVQLVESGGGLVQPGGSLKLSCAASGFTENKYAMNWV |
| | | | RQAPGKGMEWVARIRSKYNNYATYYADAVKDRFTISRDDSKNTLYLQMNNLKTEDTAV |
| | | | YYCVRAGNFGTSYISYWAYWGQGTLVTVSSGGGGSGGGGSGGGGSQTVVTQEPSLTVS |
| | | | PGGTVTITCGSSTGAVTSGNYPNWVQKKPGQAPRGLIGGTKFLAPGTPARFSGSLSGG |
| | | | KAALTLSGVQPEDEAEYYCVLWYSNRWVFGSGTKLTVLGGGGDKTHTCPPCPAPELLG |
| | | | GPSVFLFPPKPKDTLMISRTPEVTCVVVDVSHEDPEVKENWYVDGVEVHNAKTKPCEE |
| | | | QYGSTYRCVSVLTVLHQDWLNGKEYKCKVSNKALPAPIEKTISKAKGQPREPQVYTLP |
| | | | PSREEMTKNQVSLTCLVKGFYPSDIAVEWESNGQPENNYKTTPPVLDSDGSFFLYSKL |
| | | | TVDKSRWQQGNVFSCSVMHEALHNHYTQKSLSLSPGKGGGGSGGGGSGGGGSGGGGSG |
| | | | GGGSGGGGSDKTHTCPPCPAPELLGGPSVFLFPPKPKDTLMISRTPEVTCVVVDVSHE |
| | | | DPEVKFNWYVDGVEVHNAKTKPCEEQYGSTYRCVSVLTVLHQDWLNGKEYKCKVSNKA |
| | | | LPAPIEKTISKAKGQPREPQVYTLPPSREEMTKNQVSLTCLVKGFYPSDIAVEWESNG |
| | | | QPENNYKTTPPVLDSDGSFFLYSKLTVDKSRWQQGNVFSCSVMHEALHNHYTQKSLSL |
| | | | SPGK |
| 2110 | CD123 24- | artificial | EVQLLESGGGLVQPGGSLRLSCAASGFTFSHYAMSWVRQAPGKCLEWVSAVSGGGDKT |
| | B4-f NK CC | | LYADAVKGRFTISRDNSKNTLFLQMNSLRAEDTAIYYCARLRGFYYGMDVWGQGTTVT |
| | x I2M2 X | | VSSGGGGSGGGGSGGGGSDIVLTQSPLSLPVTPGEPASISCRSSQSLLHSNKYNYLDW |
| | ScFc-Full | | YLQKPGQSPQLLIYLGSNRASGVPDRFSGSGSGTDFTLKISRVEAEDVGVYYCMQALQ |
| | Sequence | | TPPITFGCGTRLEIKSGGGGSEVQLVESGGGLVQPGGSLKLSCAASGFTENKYAINWV |
| | | | REAPGKGLEWVARIRSKYNNYATYYADAVKDRFTISRDDSKNTAYLQMNNLKTEDTAV |
| | | | YYCVRNANFGTSYISYFAYWGQGTLVTVSSGGGGSGGGGSGGGGSQTVVTQEPSLTVS |
| | | | PGGTVTLTCGSSTGAVTSGNYPNWVQKKPGQAPRGLIGGTKFLAPGTPARFSGSLLGG |
| | | | KAALTLSGVQPEDEAEYYCVLWYSNRWVFGSGTKLTVLGGGGDKTHTCPPCPAPELLG |
| | | | GPSVFLFPPKPKDTLMISRTPEVTCVVVDVSHEDPEVKENWYVDGVEVHNAKTKPCEE |
| | | | QYGSTYRCVSVLTVLHQDWLNGKEYKCKVSNKALPAPIEKTISKAKGQPREPQVYTLP |
| | | | PSREEMTKNQVSLTCLVKGFYPSDIAVEWESNGQPENNYKTTPPVLDSDGSFFLYSKL |
| | | | TVDKSRWQQGNVFSCSVMHEALHNHYTQKSLSLSPGKGGGGSGGGGSGGGGSGGGGSG |
| | | | GGGSGGGGSDKTHTCPPCPAPELLGGPSVFLFPPKPKDTLMISRTPEVTCVVVDVSHE |
| | | | DPEVKFNWYVDGVEVHNAKTKPCEEQYGSTYRCVSVLTVLHQDWLNGKEYKCKVSNKA |
| | | | LPAPIEKTISKAKGQPREPQVYTLPPSREEMTKNQVSLTCLVKGFYPSDIAVEWESNG |
| | | | QPENNYKTTPPVLDSDGSFFLYSKLTVDKSRWQQGNVFSCSVMHEALHNHYTQKSLSL |
| | | | SPGK |
| 2111 | CD19 97- | artificial | SYGMH |
| | G1RE-C2 LH | | |
| | CC-HCDR1 | | |
| 2112 | CD19 97- | artificial | VISYEGSNKYYAESVKG |
| | G1RE-C2 LH | | |
| | CC-HCDR2 | | |
| 2113 | CD19 97- | artificial | DRGTIFGNYGLEV |
| | G1RE-C2 LH | | |
| | CC-HCDR3 | | |
| 2114 | CD19 97- | artificial | RSSQSLLHKNAFNYLD |
| | G1RE-C2 LH | | |
| | CC-LCDR1 | | |
| 2115 | CD19 97- | artificial | LGSNRAS |
| | G1RE-C2 LH | | |
| | CC-LCDR2 | | |
| 2116 | CD19 97- | artificial | MQALQTPFT |
| | G1RE-C2 LH | | |
| | CC-LCDR3 | | |
| 2117 | CD19 97- | artificial | QVQLVESGGGVVQPGRSLRLSCAASGFTFSSYGMHWVRQAPGKCLEWVAVISYEGSNK |
| | G1RE-C2 LH | | YYAESVKGRFTISRDNSKNTLYLQMNSLRDEDTAVYYCARDRGTIFGNYGLEVWGQGT |
| | CC-VH | | TVTVSS |
| 2118 | CD19 97- | artificial | DIVMTQSPLSLPVISGEPASISCRSSQSLLHKNAFNYLDWYLQKPGQSPQLLIYLGSN |
| | G1RE-C2 LH | | RASGVPDRFSGSGSGTDFTLKISRVEAEDVGVYYCMQALQTPFTFGCGTKVDIK |
| | CC-VL | | |
| 2119 | CD19 97- | artificial | EIVMTQSPLSLPVISGEPASISCRSSQSLLHKNAFNYLDWYLQKPGQSPQLLIYLGSN |
| | G1RE-C2 LH | | RASGVPDRFSGSGSGTDFTLKISRVEAEDVGVYYCMQALQTPFTFGCGTKVDIK |
| | CC EI GQ- | | |
| | VL | | |
| 2120 | CD19 97- | artificial | DIVMTQSPLSLPVISGEPASISCRSSQSLLHKNAFNYLDWYLQKPGQSPQLLIYLGSN |
| | G1RE-C2 LH | | RASGVPDRFSGSGSGTDFTLKISRVEAEDVGVYYCMQALQTPFTFGCGTKVDIKGGGG |
| | CC x I2C x | | SGGGGSGGGGSQVQLVESGGGVVQPGRSLRLSCAASGFTFSSYGMHWVRQAPGKCLEW |
| | scFc-Full | | VAVISYEGSNKYYAESVKGRFTISRDNSKNTLYLQMNSLRDEDTAVYYCARDRGTIFG |
| | Sequence | | NYGLEVWGQGTTVTVSSSGGGGSEVQLVESGGGLVQPGGSLKLSCAASGFTENKYAMN |
| | | | WVRQAPGKGLEWVARIRSKYNNYATYYADSVKDRFTISRDDSKNTAYLQMNNLKTEDT |
| | | | AVYYCVRHGNFGNSYISYWAYWGQGTLVTVSSGGGGSGGGGSGGGGSQTVVTQEPSLT |
| | | | VSPGGTVTLTCGSSTGAVTSGNYPNWVQQKPGQAPRGLIGGTKFLAPGTPARFSGSLL |
| | | | GGKAALTLSGVQPEDEAEYYCVLWYSNRWVFGGGTKLTVLGGGGDKTHTCPPCPAPEL |
| | | | LGGPSVFLFPPKPKDTLMISRTPEVTCVVVDVSHEDPEVKENWYVDGVEVHNAKTKPC |
| | | | EEQYGSTYRCVSVLTVLHQDWLNGKEYKCKVSNKALPAPIEKTISKAKGQPREPQVYT |
| | | | LPPSREEMTKNQVSLTCLVKGFYPSDIAVEWESNGQPENNYKTTPPVLDSDGSFFLYS |
| | | | KLTVDKSRWQQGNVFSCSVMHEALHNHYTQKSLSLSPGKGGGGSGGGGSGGGGSGGGG |
| | | | SGGGGSGGGGSDKTHTCPPCPAPELLGGPSVFLFPPKPKDTLMISRTPEVTCVVVDVS |
| | | | HEDPEVKFNWYVDGVEVHNAKTKPCEEQYGSTYRCVSVLTVLHQDWLNGKEYKCKVSN |
| | | | KALPAPIEKTISKAKGQPREPQVYTLPPSREEMTKNQVSLTCLVKGFYPSDIAVEWES |
| | | | NGQPENNYKTTPPVLDSDGSFFLYSKLTVDKSRWQQGNVFSCSVMHEALHNHYTQKSL |
| | | | SLSPGK |
| 2121 | CD19 97- | artificial | DIVMTQSPLSLPVISGEPASISCRSSQSLLHKNAFNYLDWYLQKPGQSPQLLIYLGSN |
| | G1RE-C2 LH | | RASGVPDRFSGSGSGTDFTLKISRVEAEDVGVYYCMQALQTPFTFGCGTKVDIKGGGG |
| | CC x I2E X | | SGGGGSGGGGSQVQLVESGGGVVQPGRSLRLSCAASGFTFSSYGMHWVRQAPGKCLEW |
| | scFc-Full | | VAVISYEGSNKYYAESVKGRFTISRDNSKNTLYLQMNSLRDEDTAVYYCARDRGTIFG |
| | Sequence | | NYGLEVWGQGTTVTVSSSGGGGSEVQLVESGGGLVQPGGSLKLSCAASGFTENKYAIN |
| | | | WVRQAPGKGLEWVARIRSKYNNYATYYADAVKDRFTISRDDSKNTVYLQMNNLKTEDT |
| | | | AVYYCARAGNFGSSYISYWAYWGQGTLVTVSSGGGGSGGGGSGGGGSQTVVTQEPSLT |
| | | | VSPGGTVTITCGSSTGAVTSGNYPNWVQKKPGQAPRGLIGGTKFLAPGTPARFSGSLS |
| | | | GGKAALTLSGVQPEDEAEYYCVLWYSNRWVFGSGTKLTVLGGGGDKTHTCPPCPAPEL |
| | | | LGGPSVFLFPPKPKDTLMISRTPEVTCVVVDVSHEDPEVKENWYVDGVEVHNAKTKPC |
| | | | EEQYGSTYRCVSVLTVLHQDWLNGKEYKCKVSNKALPAPIEKTISKAKGQPREPQVYT |
| | | | LPPSREEMTKNQVSLTCLVKGFYPSDIAVEWESNGQPENNYKTTPPVLDSDGSFFLYS |
| | | | KLTVDKSRWQQGNVFSCSVMHEALHNHYTQKSLSLSPGKGGGGSGGGGSGGGGSGGGG |
| | | | SGGGGSGGGGSDKTHTCPPCPAPELLGGPSVFLFPPKPKDTLMISRTPEVTCVVVDVS |
| | | | HEDPEVKFNWYVDGVEVHNAKTKPCEEQYGSTYRCVSVLTVLHQDWLNGKEYKCKVSN |
| | | | KALPAPIEKTISKAKGQPREPQVYTLPPSREEMTKNQVSLTCLVKGFYPSDIAVEWES |
| | | | NGQPENNYKTTPPVLDSDGSFFLYSKLTVDKSRWQQGNVFSCSVMHEALHNHYTQKSL |
| | | | SLSPGK |
| 2122 | CD19 97- | artificial | DIVMTQSPLSLPVISGEPASISCRSSQSLLHKNAFNYLDWYLQKPGQSPQLLIYLGSN |
| | G1RE-C2 LH | | RASGVPDRFSGSGSGTDFTLKISRVEAEDVGVYYCMQALQTPFTFGCGTKVDIKGGGG |
| | CC x I2K X | | SGGGGSGGGGSQVQLVESGGGVVQPGRSLRLSCAASGFTFSSYGMHWVRQAPGKCLEW |
| | scFc-Full | | VAVISYEGSNKYYAESVKGRFTISRDNSKNTLYLQMNSLRDEDTAVYYCARDRGTIFG |
| | Sequence | | NYGLEVWGQGTTVTVSSSGGGGSEVQLVESGGGLVQPGGSLKLSCAASGFTENKYAMN |
| | | | WVRQAPGKGLEWVARIRSKYNNYATYYAEAVKGRFTISRDDSKNTVYLQMNNLKTEDT |
| | | | AVYYCVRNENIGTSYISYWAYWGQGTLVTVSSGGGGSGGGGSGGGGSQTVVTQEPSLT |
| | | | VSPGGTVTITCGSSTGAVTSGNYPNWVQQKPGQAPRGLIGGTKFLAPGTPARFSGSLS |
| | | | GGKAALTLSGVQPEDEAEYYCVLWYSNRWVFGSGTKLTVLGGGGDKTHTCPPCPAPEL |
| | | | LGGPSVFLFPPKPKDTLMISRTPEVTCVVVDVSHEDPEVKENWYVDGVEVHNAKTKPC |
| | | | EEQYGSTYRCVSVLTVLHQDWLNGKEYKCKVSNKALPAPIEKTISKAKGQPREPQVYT |
| | | | LPPSREEMTKNQVSLTCLVKGFYPSDIAVEWESNGQPENNYKTTPPVLDSDGSFFLYS |
| | | | KLTVDKSRWQQGNVFSCSVMHEALHNHYTQKSLSLSPGKGGGGSGGGGSGGGGSGGGG |
| | | | SGGGGSGGGGSDKTHTCPPCPAPELLGGPSVFLFPPKPKDTLMISRTPEVTCVVVDVS |
| | | | HEDPEVKFNWYVDGVEVHNAKTKPCEEQYGSTYRCVSVLTVLHQDWLNGKEYKCKVSN |
| | | | KALPAPIEKTISKAKGQPREPQVYTLPPSREEMTKNQVSLTCLVKGFYPSDIAVEWES |
| | | | NGQPENNYKTTPPVLDSDGSFFLYSKLTVDKSRWQQGNVFSCSVMHEALHNHYTQKSL |
| | | | SLSPGK |
| 2123 | CD19 97- | artificial | DIVMTQSPLSLPVISGEPASISCRSSQSLLHKNAFNYLDWYLQKPGQSPQLLIYLGSN |
| | G1RE-C2 LH | | RASGVPDRFSGSGSGTDFTLKISRVEAEDVGVYYCMQALQTPFTFGCGTKVDIKGGGG |
| | CC x I2L x | | SGGGGSGGGGSQVQLVESGGGVVQPGRSLRLSCAASGFTFSSYGMHWVRQAPGKCLEW |
| | scFc-Full | | VAVISYEGSNKYYAESVKGRFTISRDNSKNTLYLQMNSLRDEDTAVYYCARDRGTIFG |
| | Sequence | | NYGLEVWGQGTTVTVSSSGGGGSEVQLVESGGGLVQPGGSLKLSCAASGFTENKYAMN |
| | | | WVRQAPGKGMEWVARIRSKYNNYATYYADAVKDRFTISRDDSKNTLYLQMNNLKTEDT |
| | | | AVYYCVRAGNFGSSYISYFAYWGQGTLVTVSSGGGGSGGGGSGGGGSQTVVTQEPSLT |
| | | | VSPGGTVTITCGSSTGAVTSGNYPNWIQKKPGQAPRGLIGGTKFLAPGTPARFSGSLE |
| | | | GGKAALTLSGVQPEDEAEYYCVLYYSNRWVFGSGTKLTVLGGGGDKTHTCPPCPAPEL |
| | | | LGGPSVFLFPPKPKDTLMISRTPEVTCVVVDVSHEDPEVKFNWYVDGVEVHNAKTKPC |
| | | | EEQYGSTYRCVSVLTVLHQDWLNGKEYKCKVSNKALPAPIEKTISKAKGQPREPQVYT |
| | | | LPPSREEMTKNQVSLTCLVKGFYPSDIAVEWESNGQPENNYKTTPPVLDSDGSFFLYS |
| | | | KLTVDKSRWQQGNVFSCSVMHEALHNHYTQKSLSLSPGKGGGGSGGGGSGGGGSGGGG |
| | | | SGGGGSGGGGSDKTHTCPPCPAPELLGGPSVFLFPPKPKDTLMISRTPEVTCVVVDVS |
| | | | HEDPEVKFNWYVDGVEVHNAKTKPCEEQYGSTYRCVSVLTVLHQDWLNGKEYKCKVSN |
| | | | KALPAPIEKTISKAKGQPREPQVYTLPPSREEMTKNQVSLTCLVKGFYPSDIAVEWES |
| | | | NGQPENNYKTTPPVLDSDGSFFLYSKLTVDKSRWQQGNVFSCSVMHEALHNHYTQKSL |
| | | | SLSPGK |
| 2124 | CD19 97- | artificial | DIVMTQSPLSLPVISGEPASISCRSSQSLLHKNAFNYLDWYLQKPGQSPQLLIYLGSN |
| | G1RE-C2 LH | | RASGVPDRFSGSGSGTDFTLKISRVEAEDVGVYYCMQALQTPFTFGCGTKVDIKGGGG |
| | CC x I2M X | | SGGGGSGGGGSQVQLVESGGGVVQPGRSLRLSCAASGFTFSSYGMHWVRQAPGKCLEW |
| | scFc-Full | | VAVISYEGSNKYYAESVKGRFTISRDNSKNTLYLQMNSLRDEDTAVYYCARDRGTIFG |
| | Sequence | | NYGLEVWGQGTTVTVSSSGGGGSEVQLVESGGGLVQPGGSLKLSCAASGFTENKYAMN |
| | | | WVRQAPGKGMEWVARIRSKYNNYATYYADAVKDRFTISRDDSKNTLYLQMNNLKTEDT |
| | | | AVYYCVRAGNFGTSYISYWAYWGQGTLVTVSSGGGGSGGGGSGGGGSQTVVTQEPSLT |
| | | | VSPGGTVTITCGSSTGAVTSGNYPNWVQKKPGQAPRGLIGGTKFLAPGTPARFSGSLS |
| | | | GGKAALTLSGVQPEDEAEYYCVLWYSNRWVFGSGTKLTVLGGGGDKTHTCPPCPAPEL |
| | | | LGGPSVFLFPPKPKDTLMISRTPEVTCVVVDVSHEDPEVKENWYVDGVEVHNAKTKPC |
| | | | EEQYGSTYRCVSVLTVLHQDWLNGKEYKCKVSNKALPAPIEKTISKAKGQPREPQVYT |
| | | | LPPSREEMTKNQVSLTCLVKGFYPSDIAVEWESNGQPENNYKTTPPVLDSDGSFFLYS |
| | | | KLTVDKSRWQQGNVFSCSVMHEALHNHYTQKSLSLSPGKGGGGSGGGGSGGGGSGGGG |
| | | | SGGGGSGGGGSDKTHTCPPCPAPELLGGPSVFLFPPKPKDTLMISRTPEVTCVVVDVS |
| | | | HEDPEVKFNWYVDGVEVHNAKTKPCEEQYGSTYRCVSVLTVLHQDWLNGKEYKCKVSN |
| | | | KALPAPIEKTISKAKGQPREPQVYTLPPSREEMTKNQVSLTCLVKGFYPSDIAVEWES |
| | | | NGQPENNYKTTPPVLDSDGSFFLYSKLTVDKSRWQQGNVFSCSVMHEALHNHYTQKSL |
| | | | SLSPGK |
| 2125 | CD19 97- | artificial | DIVMTQSPLSLPVISGEPASISCRSSQSLLHKNAFNYLDWYLQKPGQSPQLLIYLGSN |
| | G1RE-C2 LH | | RASGVPDRFSGSGSGTDFTLKISRVEAEDVGVYYCMQALQTPFTFGCGTKVDIKGGGG |
| | CC x I2M2 x | | SGGGGSGGGGSQVQLVESGGGVVQPGRSLRLSCAASGFTFSSYGMHWVRQAPGKCLEW |
| | scFc-Full | | VAVISYEGSNKYYAESVKGRFTISRDNSKNTLYLQMNSLRDEDTAVYYCARDRGTIFG |
| | Sequence | | NYGLEVWGQGTTVTVSSSGGGGSEVQLVESGGGLVQPGGSLKLSCAASGFTENKYAIN |
| | | | WVREAPGKGLEWVARIRSKYNNYATYYADAVKDRFTISRDDSKNTAYLQMNNLKTEDT |
| | | | AVYYCVRNANFGTSYISYFAYWGQGTLVTVSSGGGGSGGGGSGGGGSQTVVTQEPSLT |
| | | | VSPGGTVTLTCGSSTGAVTSGNYPNWVQKKPGQAPRGLIGGTKFLAPGTPARFSGSLL |
| | | | GGKAALTLSGVQPEDEAEYYCVLWYSNRWVFGSGTKLTVLGGGGDKTHTCPPCPAPEL |
| | | | LGGPSVFLFPPKPKDTLMISRTPEVTCVVVDVSHEDPEVKENWYVDGVEVHNAKTKPC |
| | | | EEQYGSTYRCVSVLTVLHQDWLNGKEYKCKVSNKALPAPIEKTISKAKGQPREPQVYT |
| | | | LPPSREEMTKNQVSLTCLVKGFYPSDIAVEWESNGQPENNYKTTPPVLDSDGSFFLYS |
| | | | KLTVDKSRWQQGNVFSCSVMHEALHNHYTQKSLSLSPGKGGGGSGGGGSGGGGSGGGG |
| | | | SGGGGSGGGGSDKTHTCPPCPAPELLGGPSVFLFPPKPKDTLMISRTPEVTCVVVDVS |
| | | | HEDPEVKFNWYVDGVEVHNAKTKPCEEQYGSTYRCVSVLTVLHQDWLNGKEYKCKVSN |
| | | | KALPAPIEKTISKAKGQPREPQVYTLPPSREEMTKNQVSLTCLVKGFYPSDIAVEWES |
| | | | NGQPENNYKTTPPVLDSDGSFFLYSKLTVDKSRWQQGNVFSCSVMHEALHNHYTQKSL |
| | | | SLSPGK |
| 2126 | CD20 99-E5 | artificial | SYWMH |
| | CC-HCDR1 | | |
| 2127 | CD20 99-E5 | artificial | YITPSTGYTEYNQKFKG |
| | CC-HCDR2 | | |
| 2128 | CD20 99-E5 | artificial | VHDYDRAMEY |
| | CC-HCDR3 | | |
| 2129 | CD20 99-E5 | artificial | KASQDINKYIA |
| | CC-LCDR1 | | |
| 2130 | CD20 99-E5 | artificial | YTSTLQP |
| | CC-LCDR2 | | |
| 2131 | CD20 99-E5 | artificial | LQYASYPFT |
| | CC-LCDR3 | | |
| 2132 | CD20 99-E5 | artificial | QVQLVQSGAEVKKPGASVKVSCKASGYTFTSYWMHWVRQAPGQCLEWIGYITPSTGYT |
| | CC-VH | | EYNQKFKGRVTMTRDKSTSTVYMELSSLTSEDTAVYYCARVHDYDRAMEYWGQGTTVT |
| | | | VSS |
| 2133 | CD20 99-E5 | artificial | DIQMTQSPSSLSASVGDRVTITCKASQDINKYIAWYQQKPGKGPKLLIYYTSTLQPGV |
| | CC-VL | | PSRFSGSGSGTDFTFTISSLQPEDIATYYCLQYASYPFTFGCGTRLEIK |
| 2134 | CD20 99-E5 | artificial | EIQMTQSPSSLSASVGDRVTITCKASQDINKYIAWYQQKPGKGPKLLIYYTSTLQPGV |
| | CC EI GQ- | | PSRFSGSGSGTDFTFTISSLQPEDIATYYCLQYASYPFTFGCGTRLEIK |
| | VL | | |
| 2135 | CD20 99-E5 | artificial | QVQLVQSGAEVKKPGASVKVSCKASGYTFTSYWMHWVRQAPGQCLEWIGYITPSTGYT |
| | CC x I2C x | | EYNQKFKGRVTMTRDKSTSTVYMELSSLTSEDTAVYYCARVHDYDRAMEYWGQGTTVT |
| | scFc-Full | | VSSGGGGSGGGGSGGGGSDIQMTQSPSSLSASVGDRVTITCKASQDINKYIAWYQQKP |
| | Sequence | | GKGPKLLIYYTSTLQPGVPSRFSGSGSGTDFTFTISSLQPEDIATYYCLQYASYPFTF |
| | | | GCGTRLEIKSGGGGSEVQLVESGGGLVQPGGSLKLSCAASGFTENKYAMNWVRQAPGK |
| | | | GLEWVARIRSKYNNYATYYADSVKDRFTISRDDSKNTAYLQMNNLKTEDTAVYYCVRH |
| | | | GNFGNSYISYWAYWGQGTLVTVSSGGGGSGGGGSGGGGSQTVVTQEPSLTVSPGGTVT |
| | | | LTCGSSTGAVTSGNYPNWVQQKPGQAPRGLIGGTKFLAPGTPARFSGSLLGGKAALTL |
| | | | SGVQPEDEAEYYCVLWYSNRWVFGGGTKLTVLGGGGDKTHTCPPCPAPELLGGPSVFL |
| | | | FPPKPKDTLMISRTPEVTCVVVDVSHEDPEVKFNWYVDGVEVHNAKTKPCEEQYGSTY |
| | | | RCVSVLTVLHQDWLNGKEYKCKVSNKALPAPIEKTISKAKGQPREPQVYTLPPSREEM |
| | | | TKNQVSLTCLVKGFYPSDIAVEWESNGQPENNYKTTPPVLDSDGSFFLYSKLTVDKSR |
| | | | WQQGNVFSCSVMHEALHNHYTQKSLSLSPGKGGGGSGGGGSGGGGSGGGGSGGGGSGG |
| | | | GGSDKTHTCPPCPAPELLGGPSVFLFPPKPKDTLMISRTPEVTCVVVDVSHEDPEVKF |
| | | | NWYVDGVEVHNAKTKPCEEQYGSTYRCVSVLTVLHQDWLNGKEYKCKVSNKALPAPIE |
| | | | KTISKAKGQPREPQVYTLPPSREEMTKNQVSLTCLVKGFYPSDIAVEWESNGQPENNY |
| | | | KTTPPVLDSDGSFFLYSKLTVDKSRWQQGNVFSCSVMHEALHNHYTQKSLSLSPGK |
| 2136 | CD20 99-E5 | artificial | QVQLVQSGAEVKKPGASVKVSCKASGYTFTSYWMHWVRQAPGQCLEWIGYITPSTGYT |
| | CC x I2E X | | EYNQKFKGRVTMTRDKSTSTVYMELSSLTSEDTAVYYCARVHDYDRAMEYWGQGTTVT |
| | scFc-Full | | VSSGGGGSGGGGSGGGGSDIQMTQSPSSLSASVGDRVTITCKASQDINKYIAWYQQKP |
| | Sequence | | GKGPKLLIYYTSTLQPGVPSRFSGSGSGTDFTFTISSLQPEDIATYYCLQYASYPFTF |
| | | | GCGTRLEIKSGGGGSEVQLVESGGGLVQPGGSLKLSCAASGFTENKYAINWVRQAPGK |
| | | | GLEWVARIRSKYNNYATYYADAVKDRFTISRDDSKNTVYLQMNNLKTEDTAVYYCARA |
| | | | GNFGSSYISYWAYWGQGTLVTVSSGGGGSGGGGSGGGGSQTVVTQEPSLTVSPGGTVT |
| | | | ITCGSSTGAVTSGNYPNWVQKKPGQAPRGLIGGTKFLAPGTPARFSGSLSGGKAALTL |
| | | | SGVQPEDEAEYYCVLWYSNRWVFGSGTKLTVLGGGGDKTHTCPPCPAPELLGGPSVFL |
| | | | FPPKPKDTLMISRTPEVTCVVVDVSHEDPEVKFNWYVDGVEVHNAKTKPCEEQYGSTY |
| | | | RCVSVLTVLHQDWLNGKEYKCKVSNKALPAPIEKTISKAKGQPREPQVYTLPPSREEM |
| | | | TKNQVSLTCLVKGFYPSDIAVEWESNGQPENNYKTTPPVLDSDGSFFLYSKLTVDKSR |
| | | | WQQGNVFSCSVMHEALHNHYTQKSLSLSPGKGGGGSGGGGSGGGGSGGGGSGGGGSGG |
| | | | GGSDKTHTCPPCPAPELLGGPSVFLFPPKPKDTLMISRTPEVTCVVVDVSHEDPEVKF |
| | | | NWYVDGVEVHNAKTKPCEEQYGSTYRCVSVLTVLHQDWLNGKEYKCKVSNKALPAPIE |
| | | | KTISKAKGQPREPQVYTLPPSREEMTKNQVSLTCLVKGFYPSDIAVEWESNGQPENNY |
| | | | KTTPPVLDSDGSFFLYSKLTVDKSRWQQGNVFSCSVMHEALHNHYTQKSLSLSPGK |
| 2137 | CD20 99-E5 | artificial | QVQLVQSGAEVKKPGASVKVSCKASGYTFTSYWMHWVRQAPGQCLEWIGYITPSTGYT |
| | CC x I2K x | | EYNQKFKGRVTMTRDKSTSTVYMELSSLTSEDTAVYYCARVHDYDRAMEYWGQGTTVT |
| | scFc-Full | | VSSGGGGSGGGGSGGGGSDIQMTQSPSSLSASVGDRVTITCKASQDINKYIAWYQQKP |
| | Sequence | | GKGPKLLIYYTSTLQPGVPSRFSGSGSGTDFTFTISSLQPEDIATYYCLQYASYPFTF |
| | | | GCGTRLEIKSGGGGSEVQLVESGGGLVQPGGSLKLSCAASGFTFNKYAMNWVRQAPGK |
| | | | GLEWVARIRSKYNNYATYYAEAVKGRFTISRDDSKNTVYLQMNNLKTEDTAVYYCVRN |
| | | | ENIGTSYISYWAYWGQGTLVTVSSGGGGSGGGGSGGGGSQTVVTQEPSLTVSPGGTVT |
| | | | ITCGSSTGAVTSGNYPNWVQQKPGQAPRGLIGGTKFLAPGTPARFSGSLSGGKAALTL |
| | | | SGVQPEDEAEYYCVLWYSNRWVFGSGTKLTVLGGGGDKTHTCPPCPAPELLGGPSVFL |
| | | | FPPKPKDTLMISRTPEVTCVVVDVSHEDPEVKFNWYVDGVEVHNAKTKPCEEQYGSTY |
| | | | RCVSVLTVLHQDWLNGKEYKCKVSNKALPAPIEKTISKAKGQPREPQVYTLPPSREEM |
| | | | TKNQVSLTCLVKGFYPSDIAVEWESNGQPENNYKTTPPVLDSDGSFFLYSKLTVDKSR |
| | | | WQQGNVFSCSVMHEALHNHYTQKSLSLSPGKGGGGSGGGGSGGGGSGGGGSGGGGSGG |
| | | | GGSDKTHTCPPCPAPELLGGPSVFLFPPKPKDTLMISRTPEVTCVVVDVSHEDPEVKF |
| | | | NWYVDGVEVHNAKTKPCEEQYGSTYRCVSVLTVLHQDWLNGKEYKCKVSNKALPAPIE |
| | | | KTISKAKGQPREPQVYTLPPSREEMTKNQVSLTCLVKGFYPSDIAVEWESNGQPENNY |
| | | | KTTPPVLDSDGSFFLYSKLTVDKSRWQQGNVFSCSVMHEALHNHYTQKSLSLSPGK |
| 2138 | CD20 99-E5 | artificial | QVQLVQSGAEVKKPGASVKVSCKASGYTFTSYWMHWVRQAPGQCLEWIGYITPSTGYT |
| | CC x I2L x | | EYNQKFKGRVTMTRDKSTSTVYMELSSLTSEDTAVYYCARVHDYDRAMEYWGQGTTVT |
| | scFc-Full | | VSSGGGGSGGGGSGGGGSDIQMTQSPSSLSASVGDRVTITCKASQDINKYIAWYQQKP |
| | Sequence | | GKGPKLLIYYTSTLQPGVPSRFSGSGSGTDFTFTISSLQPEDIATYYCLQYASYPFTF |
| | | | GCGTRLEIKSGGGGSEVQLVESGGGLVQPGGSLKLSCAASGFTENKYAMNWVRQAPGK |
| | | | GMEWVARIRSKYNNYATYYADAVKDRFTISRDDSKNTLYLQMNNLKTEDTAVYYCVRA |
| | | | GNFGSSYISYFAYWGQGTLVTVSSGGGGSGGGGSGGGGSQTVVTQEPSLTVSPGGTVT |
| | | | ITCGSSTGAVTSGNYPNWIQKKPGQAPRGLIGGTKFLAPGTPARFSGSLEGGKAALTL |
| | | | SGVQPEDEAEYYCVLYYSNRWVFGSGTKLTVLGGGGDKTHTCPPCPAPELLGGPSVFL |
| | | | FPPKPKDTLMISRTPEVTCVVVDVSHEDPEVKENWYVDGVEVHNAKTKPCEEQYGSTY |
| | | | RCVSVLTVLHQDWLNGKEYKCKVSNKALPAPIEKTISKAKGQPREPQVYTLPPSREEM |
| | | | TKNQVSLTCLVKGFYPSDIAVEWESNGQPENNYKTTPPVLDSDGSFFLYSKLTVDKSR |
| | | | WQQGNVFSCSVMHEALHNHYTQKSLSLSPGKGGGGSGGGGSGGGGSGGGGSGGGGSGG |
| | | | GGSDKTHTCPPCPAPELLGGPSVFLFPPKPKDTLMISRTPEVTCVVVDVSHEDPEVKF |
| | | | NWYVDGVEVHNAKTKPCEEQYGSTYRCVSVLTVLHQDWLNGKEYKCKVSNKALPAPIE |
| | | | KTISKAKGQPREPQVYTLPPSREEMTKNQVSLTCLVKGFYPSDIAVEWESNGQPENNY |
| | | | KTTPPVLDSDGSFFLYSKLTVDKSRWQQGNVFSCSVMHEALHNHYTQKSLSLSPGK |
| | | | QVQLVQSGAEVKKPGASVKVSCKASGYTFTSYWMHWVRQAPGQCLEWIGYITPSTGYT |
| 2139 | CD20 99-E5 | artificial | EYNQKFKGRVTMTRDKSTSTVYMELSSLTSEDTAVYYCARVHDYDRAMEYWGQGTTVT |
| | CC x I2M X | | VSSGGGGSGGGGSGGGGSDIQMTQSPSSLSASVGDRVTITCKASQDINKYIAWYQQKP |
| | scFc-Full | | GKGPKLLIYYTSTLQPGVPSRFSGSGSGTDFTFTISSLQPEDIATYYCLQYASYPFTF |
| | Sequence | | GCGTRLEIKSGGGGSEVQLVESGGGLVQPGGSLKLSCAASGFTFNKYAMNWVRQAPGK |
| | | | GMEWVARIRSKYNNYATYYADAVKDRFTISRDDSKNTLYLQMNNLKTEDTAVYYCVRA |
| | | | GNFGTSYISYWAYWGQGTLVTVSSGGGGSGGGGSGGGGSQTVVTQEPSLTVSPGGTVT |
| | | | ITCGSSTGAVTSGNYPNWVQKKPGQAPRGLIGGTKFLAPGTPARFSGSLSGGKAALTL |
| | | | SGVQPEDEAEYYCVLWYSNRWVFGSGTKLTVLGGGGDKTHTCPPCPAPELLGGPSVEL |
| | | | FPPKPKDTLMISRTPEVTCVVVDVSHEDPEVKFNWYVDGVEVHNAKTKPCEEQYGSTY |
| | | | RCVSVLTVLHQDWLNGKEYKCKVSNKALPAPIEKTISKAKGQPREPQVYTLPPSREEM |
| | | | TKNQVSLTCLVKGFYPSDIAVEWESNGQPENNYKTTPPVLDSDGSFFLYSKLTVDKSR |
| | | | WQQGNVFSCSVMHEALHNHYTQKSLSLSPGKGGGGSGGGGSGGGGSGGGGSGGGGSGG |
| | | | GGSDKTHTCPPCPAPELLGGPSVFLFPPKPKDTLMISRTPEVTCVVVDVSHEDPEVKF |
| | | | NWYVDGVEVHNAKTKPCEEQYGSTYRCVSVLTVLHQDWLNGKEYKCKVSNKALPAPIE |
| | | | KTISKAKGQPREPQVYTLPPSREEMTKNQVSLTCLVKGFYPSDIAVEWESNGQPENNY |
| | | | KTTPPVLDSDGSFFLYSKLTVDKSRWQQGNVFSCSVMHEALHNHYTQKSLSLSPGK |
| 2140 | CD20 99-E5 | artificial | QVQLVQSGAEVKKPGASVKVSCKASGYTFTSYWMHWVRQAPGQCLEWIGYITPSTGYT |
| | CC x I2M2 x | | EYNQKFKGRVTMTRDKSTSTVYMELSSLTSEDTAVYYCARVHDYDRAMEYWGQGTTVT |
| | scFc-Full | | VSSGGGGSGGGGSGGGGSDIQMTQSPSSLSASVGDRVTITCKASQDINKYIAWYQQKP |
| | Sequence | | GKGPKLLIYYTSTLQPGVPSRFSGSGSGTDFTFTISSLQPEDIATYYCLQYASYPFTF |
| | | | GCGTRLEIKSGGGGSEVQLVESGGGLVQPGGSLKLSCAASGFTFNKYAINWVREAPGK |
| | | | GLEWVARIRSKYNNYATYYADAVKDRFTISRDDSKNTAYLQMNNLKTEDTAVYYCVRN |
| | | | ANFGTSYISYFAYWGQGTLVTVSSGGGGSGGGGSGGGGSQTVVTQEPSLTVSPGGTVT |
| | | | LTCGSSTGAVTSGNYPNWVQKKPGQAPRGLIGGTKFLAPGTPARFSGSLLGGKAALTL |
| | | | SGVQPEDEAEYYCVLWYSNRWVFGSGTKLTVLGGGGDKTHTCPPCPAPELLGGPSVFL |
| | | | FPPKPKDTLMISRTPEVTCVVVDVSHEDPEVKFNWYVDGVEVHNAKTKPCEEQYGSTY |
| | | | RCVSVLTVLHQDWLNGKEYKCKVSNKALPAPIEKTISKAKGQPREPQVYTLPPSREEM |
| | | | TKNQVSLTCLVKGFYPSDIAVEWESNGQPENNYKTTPPVLDSDGSFFLYSKLTVDKSR |
| | | | WQQGNVFSCSVMHEALHNHYTQKSLSLSPGKGGGGSGGGGSGGGGSGGGGSGGGGSGG |
| | | | GGSDKTHTCPPCPAPELLGGPSVFLFPPKPKDTLMISRTPEVTCVVVDVSHEDPEVKE |
| | | | NWYVDGVEVHNAKTKPCEEQYGSTYRCVSVLTVLHQDWLNGKEYKCKVSNKALPAPIE |
| | | | KTISKAKGQPREPQVYTLPPSREEMTKNQVSLTCLVKGFYPSDIAVEWESNGQPENNY |
| | | | KTTPPVLDSDGSFFLYSKLTVDKSRWQQGNVFSCSVMHEALHNHYTQKSLSLSPGK |
| 2141 | CD22 28- | artificial | SYGIS |
| | B7N65S CC- | | |
| | HCDR1 | | |
| 2142 | CD22 28- | artificial | WISAYSGNAIYAQKLQG |
| | B7N65S CC- | | |
| | HCDR2 | | |
| 2143 | CD22 28- | artificial | DPDYYGSGSYSDY |
| | B7N65S CC- | | |
| | HCDR3 | | |
| 2144 | CD22 28- | artificial | RASQSVSSNLA |
| | B7N65S CC- | | |
| | LCDR1 | | |
| 2145 | CD22 28- | artificial | GASSRAT |
| | B7N65S CC- | | |
| | LCDR2 | | |
| 2146 | CD22 28- | artificial | QQYHSWPLLT |
| | B7N65S CC- | | |
| | LCDR3 | | |
| 2147 | CD22 28- | artificial | QVQLVQSGAEVKKPGASVKVSCKASGYTFTSYGISWVRQAPGQCLEWMGWISAYSGNA |
| | B7N65S CC- | | IYAQKLQGRVTMTRDTSTSTAYMELRSLRSDDTAVYYCARDPDYYGSGSYSDYWGQGT |
| | VH | | LVTVSS |
| 2148 | CD22 28- | artificial | EIVLTQSPATLSVSPGERATLSCRASQSVSSNLAWYQQKPGQAPRLLIYGASSRATGI |
| | B7N65S CC- | | PARFSGSGSGTEFTLTISSLQSEDFAVYYCQQYHSWPLLTFGCGTKVEIK |
| | VL | | |
| 2149 | CD22 28-B7 | artificial | QVQLVQSGAEVKKPGASVKVSCKASGYTFTSYGISWVRQAPGQCLEWMGWISAYSGNA |
| | N65S CC X | | IYAQKLQGRVTMTRDTSTSTAYMELRSLRSDDTAVYYCARDPDYYGSGSYSDYWGQGT |
| | I2C x scFc | | LVTVSSGGGGSGGGGSGGGGSEIVLTQSPATLSVSPGERATLSCRASQSVSSNLAWYQ |
| | -Full | | QKPGQAPRLLIYGASSRATGIPARFSGSGSGTEFTLTISSLQSEDFAVYYCQQYHSWP |
| | Sequence | | LLTFGCGTKVEIKSGGGGSEVQLVESGGGLVQPGGSLKLSCAASGFTFNKYAMNWVRQ |
| | | | APGKGLEWVARIRSKYNNYATYYADSVKDRFTISRDDSKNTAYLQMNNLKTEDTAVYY |
| | | | CVRHGNFGNSYISYWAYWGQGTLVTVSSGGGGSGGGGSGGGGSQTVVTQEPSLTVSPG |
| | | | GTVTLTCGSSTGAVTSGNYPNWVQQKPGQAPRGLIGGTKFLAPGTPARFSGSLLGGKA |
| | | | ALTLSGVQPEDEAEYYCVLWYSNRWVFGGGTKLTVLGGGGDKTHTCPPCPAPELLGGP |
| | | | SVFLFPPKPKDTLMISRTPEVTCVVVDVSHEDPEVKFNWYVDGVEVHNAKTKPCEEQY |
| | | | GSTYRCVSVLTVLHQDWLNGKEYKCKVSNKALPAPIEKTISKAKGQPREPQVYTLPPS |
| | | | REEMTKNQVSLTCLVKGFYPSDIAVEWESNGQPENNYKTTPPVLDSDGSFFLYSKLTV |
| | | | DKSRWQQGNVFSCSVMHEALHNHYTQKSLSLSPGKGGGGSGGGGSGGGGSGGGGSGGG |
| | | | GSGGGGSDKTHTCPPCPAPELLGGPSVFLFPPKPKDTLMISRTPEVTCVVVDVSHEDP |
| | | | EVKFNWYVDGVEVHNAKTKPCEEQYGSTYRCVSVLTVLHQDWLNGKEYKCKVSNKALP |
| | | | APIEKTISKAKGQPREPQVYTLPPSREEMTKNQVSLTCLVKGFYPSDIAVEWESNGQP |
| | | | ENNYKTTPPVLDSDGSFFLYSKLTVDKSRWQQGNVFSCSVMHEALHNHYTQKSLSLSP |
| | | | GK |
| 2150 | CD22 28-B7 | artificial | QVQLVQSGAEVKKPGASVKVSCKASGYTFTSYGISWVRQAPGQCLEWMGWISAYSGNA |
| | N65S CC X | | IYAQKLQGRVTMTRDTSTSTAYMELRSLRSDDTAVYYCARDPDYYGSGSYSDYWGQGT |
| | I2E x scFc | | LVTVSSGGGGSGGGGSGGGGSEIVLTQSPATLSVSPGERATLSCRASQSVSSNLAWYQ |
| | -Full | | QKPGQAPRLLIYGASSRATGIPARFSGSGSGTEFTLTISSLQSEDFAVYYCQQYHSWP |
| | Sequence | | LLTFGCGTKVEIKSGGGGSEVQLVESGGGLVQPGGSLKLSCAASGFTENKYAINWVRQ |
| | | | APGKGLEWVARIRSKYNNYATYYADAVKDRFTISRDDSKNTVYLQMNNLKTEDTAVYY |
| | | | CARAGNFGSSYISYWAYWGQGTLVTVSSGGGGSGGGGSGGGGSQTVVTQEPSLTVSPG |
| | | | GTVTITCGSSTGAVTSGNYPNWVQKKPGQAPRGLIGGTKFLAPGTPARFSGSLSGGKA |
| | | | ALTLSGVQPEDEAEYYCVLWYSNRWVFGSGTKLTVLGGGGDKTHTCPPCPAPELLGGP |
| | | | SVFLFPPKPKDTLMISRTPEVTCVVVDVSHEDPEVKFNWYVDGVEVHNAKTKPCEEQY |
| | | | GSTYRCVSVLTVLHQDWLNGKEYKCKVSNKALPAPIEKTISKAKGQPREPQVYTLPPS |
| | | | REEMTKNQVSLTCLVKGFYPSDIAVEWESNGQPENNYKTTPPVLDSDGSFFLYSKLTV |
| | | | DKSRWQQGNVFSCSVMHEALHNHYTQKSLSLSPGKGGGGSGGGGSGGGGSGGGGSGGG |
| | | | GSGGGGSDKTHTCPPCPAPELLGGPSVFLFPPKPKDTLMISRTPEVTCVVVDVSHEDP |
| | | | EVKFNWYVDGVEVHNAKTKPCEEQYGSTYRCVSVLTVLHQDWLNGKEYKCKVSNKALP |
| | | | APIEKTISKAKGQPREPQVYTLPPSREEMTKNQVSLTCLVKGFYPSDIAVEWESNGQP |
| | | | ENNYKTTPPVLDSDGSFFLYSKLTVDKSRWQQGNVFSCSVMHEALHNHYTQKSLSLSP |
| | | | GK |
| 2151 | CD22 28-B7 | artificial | QVQLVQSGAEVKKPGASVKVSCKASGYTFTSYGISWVRQAPGQCLEWMGWISAYSGNA |
| | N65S CC X | | IYAQKLQGRVTMTRDTSTSTAYMELRSLRSDDTAVYYCARDPDYYGSGSYSDYWGQGT |
| | I2K x scFc | | LVTVSSGGGGSGGGGSGGGGSEIVLTQSPATLSVSPGERATLSCRASQSVSSNLAWYQ |
| | -Full | | QKPGQAPRLLIYGASSRATGIPARFSGSGSGTEFTLTISSLQSEDFAVYYCQQYHSWP |
| | Sequence | | LLTFGCGTKVEIKSGGGGSEVQLVESGGGLVQPGGSLKLSCAASGFTFNKYAMNWVRQ |
| | | | APGKGLEWVARIRSKYNNYATYYAEAVKGRFTISRDDSKNTVYLQMNNLKTEDTAVYY |
| | | | CVRNENIGTSYISYWAYWGQGTLVTVSSGGGGSGGGGSGGGGSQTVVTQEPSLTVSPG |
| | | | GTVTITCGSSTGAVTSGNYPNWVQQKPGQAPRGLIGGTKFLAPGTPARFSGSLSGGKA |
| | | | ALTLSGVQPEDEAEYYCVLWYSNRWVFGSGTKLTVLGGGGDKTHTCPPCPAPELLGGP |
| | | | SVFLFPPKPKDTLMISRTPEVTCVVVDVSHEDPEVKFNWYVDGVEVHNAKTKPCEEQY |
| | | | GSTYRCVSVLTVLHQDWLNGKEYKCKVSNKALPAPIEKTISKAKGQPREPQVYTLPPS |
| | | | REEMTKNQVSLTCLVKGFYPSDIAVEWESNGQPENNYKTTPPVLDSDGSFFLYSKLTV |
| | | | DKSRWQQGNVFSCSVMHEALHNHYTQKSLSLSPGKGGGGSGGGGSGGGGSGGGGSGGG |
| | | | GSGGGGSDKTHTCPPCPAPELLGGPSVFLFPPKPKDTLMISRTPEVTCVVVDVSHEDP |
| | | | EVKFNWYVDGVEVHNAKTKPCEEQYGSTYRCVSVLTVLHQDWLNGKEYKCKVSNKALP |
| | | | APIEKTISKAKGQPREPQVYTLPPSREEMTKNQVSLTCLVKGFYPSDIAVEWESNGQP |
| | | | ENNYKTTPPVLDSDGSFFLYSKLTVDKSRWQQGNVFSCSVMHEALHNHYTQKSLSLSP |
| | | | GK |
| 2152 | CD22 28-B7 | artificial | QVQLVQSGAEVKKPGASVKVSCKASGYTFTSYGISWVRQAPGQCLEWMGWISAYSGNA |
| | N65S CC X | | IYAQKLQGRVTMTRDTSTSTAYMELRSLRSDDTAVYYCARDPDYYGSGSYSDYWGQGT |
| | I2L x scFc | | LVTVSSGGGGSGGGGSGGGGSEIVLTQSPATLSVSPGERATLSCRASQSVSSNLAWYQ |
| | -Full | | QKPGQAPRLLIYGASSRATGIPARFSGSGSGTEFTLTISSLQSEDFAVYYCQQYHSWP |
| | Sequence | | LLTFGCGTKVEIKSGGGGSEVQLVESGGGLVQPGGSLKLSCAASGFTENKYAMNWVRQ |
| | | | APGKGMEWVARIRSKYNNYATYYADAVKDRFTISRDDSKNTLYLQMNNLKTEDTAVYY |
| | | | CVRAGNFGSSYISYFAYWGQGTLVTVSSGGGGSGGGGSGGGGSQTVVTQEPSLTVSPG |
| | | | GTVTITCGSSTGAVTSGNYPNWIQKKPGQAPRGLIGGTKFLAPGTPARFSGSLEGGKA |
| | | | ALTLSGVQPEDEAEYYCVLYYSNRWVFGSGTKLTVLGGGGDKTHTCPPCPAPELLGGP |
| | | | SVFLFPPKPKDTLMISRTPEVTCVVVDVSHEDPEVKENWYVDGVEVHNAKTKPCEEQY |
| | | | GSTYRCVSVLTVLHQDWLNGKEYKCKVSNKALPAPIEKTISKAKGQPREPQVYTLPPS |
| | | | REEMTKNQVSLTCLVKGFYPSDIAVEWESNGQPENNYKTTPPVLDSDGSFFLYSKLTV |
| | | | DKSRWQQGNVFSCSVMHEALHNHYTQKSLSLSPGKGGGGSGGGGSGGGGSGGGGSGGG |
| | | | GSGGGGSDKTHTCPPCPAPELLGGPSVFLFPPKPKDTLMISRTPEVTCVVVDVSHEDP |
| | | | EVKFNWYVDGVEVHNAKTKPCEEQYGSTYRCVSVLTVLHQDWLNGKEYKCKVSNKALP |
| | | | APIEKTISKAKGQPREPQVYTLPPSREEMTKNQVSLTCLVKGFYPSDIAVEWESNGQP |
| | | | ENNYKTTPPVLDSDGSFFLYSKLTVDKSRWQQGNVFSCSVMHEALHNHYTQKSLSLSP |
| | | | GK |
| 2153 | CD22 28-B7 | artificial | QVQLVQSGAEVKKPGASVKVSCKASGYTFTSYGISWVRQAPGQCLEWMGWISAYSGNA |
| | N65S CC X | | IYAQKLQGRVTMTRDTSTSTAYMELRSLRSDDTAVYYCARDPDYYGSGSYSDYWGQGT |
| | I2M x scFc | | LVTVSSGGGGSGGGGSGGGGSEIVLTQSPATLSVSPGERATLSCRASQSVSSNLAWYQ |
| | -Full | | QKPGQAPRLLIYGASSRATGIPARFSGSGSGTEFTLTISSLQSEDFAVYYCQQYHSWP |
| | Sequence | | LLTFGCGTKVEIKSGGGGSEVQLVESGGGLVQPGGSLKLSCAASGFTFNKYAMNWVRQ |
| | | | APGKGMEWVARIRSKYNNYATYYADAVKDRFTISRDDSKNTLYLQMNNLKTEDTAVYY |
| | | | CVRAGNFGTSYISYWAYWGQGTLVTVSSGGGGSGGGGSGGGGSQTVVTQEPSLTVSPG |
| | | | GTVTITCGSSTGAVTSGNYPNWVQKKPGQAPRGLIGGTKFLAPGTPARFSGSLSGGKA |
| | | | ALTLSGVQPEDEAEYYCVLWYSNRWVFGSGTKLTVLGGGGDKTHTCPPCPAPELLGGP |
| | | | SVFLFPPKPKDTLMISRTPEVTCVVVDVSHEDPEVKENWYVDGVEVHNAKTKPCEEQY |
| | | | GSTYRCVSVLTVLHQDWLNGKEYKCKVSNKALPAPIEKTISKAKGQPREPQVYTLPPS |
| | | | REEMTKNQVSLTCLVKGFYPSDIAVEWESNGQPENNYKTTPPVLDSDGSFFLYSKLTV |
| | | | DKSRWQQGNVFSCSVMHEALHNHYTQKSLSLSPGKGGGGSGGGGSGGGGSGGGGSGGG |
| | | | GSGGGGSDKTHTCPPCPAPELLGGPSVFLFPPKPKDTLMISRTPEVTCVVVDVSHEDP |
| | | | EVKFNWYVDGVEVHNAKTKPCEEQYGSTYRCVSVLTVLHQDWLNGKEYKCKVSNKALP |
| | | | APIEKTISKAKGQPREPQVYTLPPSREEMTKNQVSLTCLVKGFYPSDIAVEWESNGQP |
| | | | ENNYKTTPPVLDSDGSFFLYSKLTVDKSRWQQGNVFSCSVMHEALHNHYTQKSLSLSP |
| | | | GK |
| 2154 | CD22 28-B7 | artificial | QVQLVQSGAEVKKPGASVKVSCKASGYTFTSYGISWVRQAPGQCLEWMGWISAYSGNA |
| | N65S CC X | | IYAQKLQGRVTMTRDTSTSTAYMELRSLRSDDTAVYYCARDPDYYGSGSYSDYWGQGT |
| | I2M2 x scFc | | LVTVSSGGGGSGGGGSGGGGSEIVLTQSPATLSVSPGERATLSCRASQSVSSNLAWYQ |
| | -Full | | QKPGQAPRLLIYGASSRATGIPARFSGSGSGTEFTLTISSLQSEDFAVYYCQQYHSWP |
| | Sequence | | LLTFGCGTKVEIKSGGGGSEVQLVESGGGLVQPGGSLKLSCAASGFTENKYAINWVRE |
| | | | APGKGLEWVARIRSKYNNYATYYADAVKDRFTISRDDSKNTAYLQMNNLKTEDTAVYY |
| | | | CVRNANFGTSYISYFAYWGQGTLVTVSSGGGGSGGGGSGGGGSQTVVTQEPSLTVSPG |
| | | | GTVTLTCGSSTGAVTSGNYPNWVQKKPGQAPRGLIGGTKFLAPGTPARFSGSLLGGKA |
| | | | ALTLSGVQPEDEAEYYCVLWYSNRWVFGSGTKLTVLGGGGDKTHTCPPCPAPELLGGP |
| | | | SVFLFPPKPKDTLMISRTPEVTCVVVDVSHEDPEVKFNWYVDGVEVHNAKTKPCEEQY |
| | | | GSTYRCVSVLTVLHQDWLNGKEYKCKVSNKALPAPIEKTISKAKGQPREPQVYTLPPS |
| | | | REEMTKNQVSLTCLVKGFYPSDIAVEWESNGQPENNYKTTPPVLDSDGSFFLYSKLTV |
| | | | DKSRWQQGNVFSCSVMHEALHNHYTQKSLSLSPGKGGGGSGGGGSGGGGSGGGGSGGG |
| | | | GSGGGGSDKTHTCPPCPAPELLGGPSVFLFPPKPKDTLMISRTPEVTCVVVDVSHEDP |
| | | | EVKFNWYVDGVEVHNAKTKPCEEQYGSTYRCVSVLTVLHQDWLNGKEYKCKVSNKALP |
| | | | APIEKTISKAKGQPREPQVYTLPPSREEMTKNQVSLTCLVKGFYPSDIAVEWESNGQP |
| | | | ENNYKTTPPVLDSDGSFFLYSKLTVDKSRWQQGNVFSCSVMHEALHNHYTQKSLSLSP |
| | | | GK |
| 2155 | CD33 E11- | artificial | NYGMN |
| | HCDR1 | | |
| 2156 | CD33 E11- | artificial | WINTYTGEPTYADKFQG |
| | HCDR2 | | |
| 2157 | CD33 E11- | artificial | WSWSDGYYVYFDY |
| | HCDR3 | | |
| 2158 | CD33 E11- | artificial | KSSQSVLDSSTNKNSLA |
| | LCDR1 | | |
| 2159 | CD33 E11- | artificial | WASTRES |
| | LCDR2 | | |
| 2160 | CD33 E11- | artificial | QQSAHFPIT |
| | LCDR3 | | |
| 2161 | CD33 E11- | artificial | QVQLVQSGAEVKKPGESVKVSCKASGYTFTNYGMNWVKQAPGQGLEWMGWINTYTGEP |
| | VH | | TYADKFQGRVTMTTDTSTSTAYMEIRNLGGDDTAVYYCARWSWSDGYYVYFDYWGQGT |
| | | | SVTVSS |
| 2162 | CD33 E11- | artificial | DIVMTQSPDSLTVSLGERTTINCKSSQSVLDSSTNKNSLAWYQQKPGQPPKLLLSWAS |
| | VL | | TRESGIPDRFSGSGSGTDFTLTIDSPQPEDSATYYCQQSAHFPITFGQGTRLEIK |
| 2163 | CD33 E11 CC | artificial | QVQLVQSGAEVKKPGESVKVSCKASGYTFTNYGMNWVKQAPGQCLEWMGWINTYTGEP |
| | -VH | | TYADKFQGRVTMTTDTSTSTAYMEIRNLGGDDTAVYYCARWSWSDGYYVYFDYWGQGT |
| | | | SVTVSS |
| 2164 | CD33 E11 CC | artificial | DIVMTQSPDSLTVSLGERTTINCKSSQSVLDSSTNKNSLAWYQQKPGQPPKLLLSWAS |
| | -VL | | TRESGIPDRFSGSGSGTDFTLTIDSPQPEDSATYYCQQSAHFPITFGCGTRLEIK |
| 2165 | CD33 E11 CC | artificial | EIVMTQSPDSLTVSLGERTTINCKSSQSVLDSSTNKNSLAWYQQKPGQPPKLLLSWAS |
| | EI GQ-VL | | TRESGIPDRFSGSGSGTDFTLTIDSPQPEDSATYYCQQSAHFPITFGCGTRLEIK |
| 2166 | CD33 E11 EI | artificial | EIVMTQSPDSLTVSLGERTTINCKSSQSVLDSSTNKNSLAWYQQKPGQPPKLLLSWAS |
| | GQ-VL | | TRESGIPDRFSGSGSGTDFTLTIDSPQPEDSATYYCQQSAHFPITFGQGTRLEIK |
| 2167 | E11 x I2C- | artificial | QVQLVQSGAEVKKPGESVKVSCKASGYTFTNYGMNWVKQAPGQGLEWMGWINTYTGEP |
| | Full | | TYADKFQGRVTMTTDTSTSTAYMEIRNLGGDDTAVYYCARWSWSDGYYVYFDYWGQGT |
| | Sequence | | SVTVSSGGGGSGGGGSGGGGSDIVMTQSPDSLTVSLGERTTINCKSSQSVLDSSTNKN |
| | | | SLAWYQQKPGQPPKLLLSWASTRESGIPDRFSGSGSGTDFTLTIDSPQPEDSATYYCQ |
| | | | QSAHFPITFGQGTRLEIKSGGGGSEVQLVESGGGLVQPGGSLKLSCAASGFTENKYAM |
| | | | NWVRQAPGKGLEWVARIRSKYNNYATYYADSVKDRFTISRDDSKNTAYLQMNNLKTED |
| | | | TAVYYCVRHGNFGNSYISYWAYWGQGTLVTVSSGGGGSGGGGSGGGGSQTVVTQEPSL |
| | | | TVSPGGTVTLTCGSSTGAVTSGNYPNWVQQKPGQAPRGLIGGTKFLAPGTPARFSGSL |
| | | | LGGKAALTLSGVQPEDEAEYYCVLWYSNRWVFGGGTKLTVL |
| 2168 | E11 x I2E- | artificial | QVQLVQSGAEVKKPGESVKVSCKASGYTFTNYGMNWVKQAPGQGLEWMGWINTYTGEP |
| | Full | | TYADKFQGRVTMTTDTSTSTAYMEIRNLGGDDTAVYYCARWSWSDGYYVYFDYWGQGT |
| | Sequence | | SVTVSSGGGGSGGGGSGGGGSDIVMTQSPDSLTVSLGERTTINCKSSQSVLDSSTNKN |
| | | | SLAWYQQKPGQPPKLLLSWASTRESGIPDRFSGSGSGTDFTLTIDSPQPEDSATYYCQ |
| | | | QSAHFPITFGQGTRLEIKSGGGGSEVQLVESGGGLVQPGGSLKLSCAASGFTFNKYAI |
| | | | NWVRQAPGKGLEWVARIRSKYNNYATYYADAVKDRFTISRDDSKNTVYLQMNNLKTED |
| | | | TAVYYCARAGNFGSSYISYWAYWGQGTLVTVSSGGGGSGGGGSGGGGSQTVVTQEPSL |
| | | | TVSPGGTVTITCGSSTGAVTSGNYPNWVQKKPGQAPRGLIGGTKFLAPGTPARFSGSL |
| | | | SGGKAALTLSGVQPEDEAEYYCVLWYSNRWVFGSGTKLTVL |
| 2169 | E11 x I2K- | artificial | QVQLVQSGAEVKKPGESVKVSCKASGYTFTNYGMNWVKQAPGQGLEWMGWINTYTGEP |
| | Full | | TYADKFQGRVTMTTDTSTSTAYMEIRNLGGDDTAVYYCARWSWSDGYYVYFDYWGQGT |
| | Sequence | | SVTVSSGGGGSGGGGSGGGGSDIVMTQSPDSLTVSLGERTTINCKSSQSVLDSSTNKN |
| | | | SLAWYQQKPGQPPKLLLSWASTRESGIPDRFSGSGSGTDFTLTIDSPQPEDSATYYCQ |
| | | | QSAHFPITFGQGTRLEIKSGGGGSEVQLVESGGGLVQPGGSLKLSCAASGFTENKYAM |
| | | | NWVRQAPGKGLEWVARIRSKYNNYATYYAEAVKGRFTISRDDSKNTVYLQMNNLKTED |
| | | | TAVYYCVRNENIGTSYISYWAYWGQGTLVTVSSGGGGSGGGGSGGGGSQTVVTQEPSL |
| | | | TVSPGGTVTITCGSSTGAVTSGNYPNWVQQKPGQAPRGLIGGTKFLAPGTPARFSGSL |
| | | | SGGKAALTLSGVQPEDEAEYYCVLWYSNRWVFGSGTKLTVL |
| 2170 | E11 x I2L- | artificial | QVQLVQSGAEVKKPGESVKVSCKASGYTFTNYGMNWVKQAPGQGLEWMGWINTYTGEP |
| | Full | | TYADKFQGRVTMTTDTSTSTAYMEIRNLGGDDTAVYYCARWSWSDGYYVYFDYWGQGT |
| | Sequence | | SVTVSSGGGGSGGGGSGGGGSDIVMTQSPDSLTVSLGERTTINCKSSQSVLDSSTNKN |
| | | | SLAWYQQKPGQPPKLLLSWASTRESGIPDRFSGSGSGTDFTLTIDSPQPEDSATYYCQ |
| | | | QSAHFPITFGQGTRLEIKSGGGGSEVQLVESGGGLVQPGGSLKLSCAASGFTENKYAM |
| | | | NWVRQAPGKGMEWVARIRSKYNNYATYYADAVKDRFTISRDDSKNTLYLQMNNLKTED |
| | | | TAVYYCVRAGNFGSSYISYFAYWGQGTLVTVSSGGGGGGGGSGGGGSQTVVTQEPSL |
| | | | TVSPGGTVTITCGSSTGAVTSGNYPNWIQKKPGQAPRGLIGGTKFLAPGTPARFSGSL |
| | | | EGGKAALTLSGVQPEDEAEYYCVLYYSNRWVFGSGTKLTVL |
| 2171 | E11 x I2M- | artificial | QVQLVQSGAEVKKPGESVKVSCKASGYTFTNYGMNWVKQAPGQGLEWMGWINTYTGEP |
| | Full | | TYADKFQGRVTMTTDTSTSTAYMEIRNLGGDDTAVYYCARWSWSDGYYVYFDYWGQGT |
| | Sequence | | SVTVSSGGGGSGGGGSGGGGSDIVMTQSPDSLTVSLGERTTINCKSSQSVLDSSTNKN |
| | | | SLAWYQQKPGQPPKLLLSWASTRESGIPDRFSGSGSGTDFTLTIDSPQPEDSATYYCQ |
| | | | QSAHFPITFGQGTRLEIKSGGGGSEVQLVESGGGLVQPGGSLKLSCAASGFTFNKYAM |
| | | | NWVRQAPGKGMEWVARIRSKYNNYATYYADAVKDRFTISRDDSKNTLYLQMNNLKTED |
| | | | TAVYYCVRAGNFGTSYISYWAYWGQGTLVTVSSGGGGSGGGGSGGGGSQTVVTQEPSL |
| | | | TVSPGGTVTITCGSSTGAVTSGNYPNWVQKKPGQAPRGLIGGTKFLAPGTPARFSGSL |
| | | | SGGKAALTLSGVQPEDEAEYYCVLWYSNRWVFGSGTKLTVL |
| 2172 | E11 x I2M2 | artificial | QVQLVQSGAEVKKPGESVKVSCKASGYTFTNYGMNWVKQAPGQGLEWMGWINTYTGEP |
| | -Full | | TYADKFQGRVTMTTDTSTSTAYMEIRNLGGDDTAVYYCARWSWSDGYYVYFDYWGQGT |
| | Sequence | | SVTVSSGGGGSGGGGSGGGGSDIVMTQSPDSLTVSLGERTTINCKSSQSVLDSSTNKN |
| | | | SLAWYQQKPGQPPKLLLSWASTRESGIPDRFSGSGSGTDFTLTIDSPQPEDSATYYCQ |
| | | | QSAHFPITFGQGTRLEIKSGGGGSEVQLVESGGGLVQPGGSLKLSCAASGFTENKYAI |
| | | | NWVREAPGKGLEWVARIRSKYNNYATYYADAVKDRFTISRDDSKNTAYLQMNNLKTED |
| | | | TAVYYCVRNANFGTSYISYFAYWGQGTLVTVSSGGGGSGGGGSGGGGSQTVVTQEPSL |
| | | | TVSPGGTVTLTCGSSTGAVTSGNYPNWVQKKPGQAPRGLIGGTKFLAPGTPARFSGSL |
| | | | LGGKAALTLSGVQPEDEAEYYCVLWYSNRWVFGSGTKLTVL |
| 2173 | CD33 E11 CC | artificial | QVQLVQSGAEVKKPGESVKVSCKASGYTFTNYGMNWVKQAPGQCLEWMGWINTYTGEP |
| | x I2C x | | TYADKFQGRVTMTTDTSTSTAYMEIRNLGGDDTAVYYCARWSWSDGYYVYFDYWGQGT |
| | scFc-Full | | SVTVSSGGGGSGGGGSGGGGSDIVMTQSPDSLTVSLGERTTINCKSSQSVLDSSTNKN |
| | Sequence | | SLAWYQQKPGQPPKLLLSWASTRESGIPDRFSGSGSGTDFTLTIDSPQPEDSATYYCQ |
| | | | QSAHFPITFGCGTRLEIKSGGGGSEVQLVESGGGLVQPGGSLKLSCAASGFTENKYAM |
| | | | NWVRQAPGKGLEWVARIRSKYNNYATYYADSVKDRFTISRDDSKNTAYLQMNNLKTED |
| | | | TAVYYCVRHGNFGNSYISYWAYWGQGTLVTVSSGGGGSGGGGSGGGGSQTVVTQEPSL |
| | | | TVSPGGTVTLTCGSSTGAVTSGNYPNWVQQKPGQAPRGLIGGTKFLAPGTPARFSGSL |
| | | | LGGKAALTLSGVQPEDEAEYYCVLWYSNRWVFGGGTKLTVLGGGGDKTHTCPPCPAPE |
| | | | LLGGPSVFLFPPKPKDTLMISRTPEVTCVVVDVSHEDPEVKFNWYVDGVEVHNAKTKP |
| | | | CEEQYGSTYRCVSVLTVLHQDWLNGKEYKCKVSNKALPAPIEKTISKAKGQPREPQVY |
| | | | TLPPSREEMTKNQVSLTCLVKGFYPSDIAVEWESNGQPENNYKTTPPVLDSDGSFFLY |
| | | | SKLTVDKSRWQQGNVFSCSVMHEALHNHYTQKSLSLSPGKGGGGSGGGGSGGGGSGGG |
| | | | GSGGGGSGGGGSDKTHTCPPCPAPELLGGPSVFLFPPKPKDTLMISRTPEVTCVVVDV |
| | | | SHEDPEVKFNWYVDGVEVHNAKTKPCEEQYGSTYRCVSVLTVLHQDWLNGKEYKCKVS |
| | | | NKALPAPIEKTISKAKGQPREPQVYTLPPSREEMTKNQVSLTCLVKGFYPSDIAVEWE |
| | | | SNGQPENNYKTTPPVLDSDGSFFLYSKLTVDKSRWQQGNVFSCSVMHEALHNHYTQKS |
| | | | LSLSPGK |
| 2174 | CD33 E11 CC | artificial | QVQLVQSGAEVKKPGESVKVSCKASGYTFTNYGMNWVKQAPGQCLEWMGWINTYTGEP |
| | x I2E X | | TYADKFQGRVTMTTDTSTSTAYMEIRNLGGDDTAVYYCARWSWSDGYYVYFDYWGQGT |
| | ScFc-Full | | SVTVSSGGGGSGGGGSGGGGSDIVMTQSPDSLTVSLGERTTINCKSSQSVLDSSTNKN |
| | Sequence | | SLAWYQQKPGQPPKLLLSWASTRESGIPDRFSGSGSGTDFTLTIDSPQPEDSATYYCQ |
| | | | QSAHFPITFGCGTRLEIKSGGGGSEVQLVESGGGLVQPGGSLKLSCAASGFTENKYAI |
| | | | NWVRQAPGKGLEWVARIRSKYNNYATYYADAVKDRFTISRDDSKNTVYLQMNNLKTED |
| | | | TAVYYCARAGNFGSSYISYWAYWGQGTLVTVSSGGGGSGGGGSGGGGSQTVVTQEPSL |
| | | | TVSPGGTVTITCGSSTGAVTSGNYPNWVQKKPGQAPRGLIGGTKFLAPGTPARFSGSL |
| | | | SGGKAALTLSGVQPEDEAEYYCVLWYSNRWVFGSGTKLTVLGGGGDKTHTCPPCPAPE |
| | | | LLGGPSVFLFPPKPKDTLMISRTPEVTCVVVDVSHEDPEVKFNWYVDGVEVHNAKTKP |
| | | | CEEQYGSTYRCVSVLTVLHQDWLNGKEYKCKVSNKALPAPIEKTISKAKGQPREPQVY |
| | | | TLPPSREEMTKNQVSLTCLVKGFYPSDIAVEWESNGQPENNYKTTPPVLDSDGSFFLY |
| | | | SKLTVDKSRWQQGNVFSCSVMHEALHNHYTQKSLSLSPGKGGGGSGGGGSGGGGSGGG |
| | | | GSGGGGSGGGGSDKTHTCPPCPAPELLGGPSVFLFPPKPKDTLMISRTPEVTCVVVDV |
| | | | SHEDPEVKFNWYVDGVEVHNAKTKPCEEQYGSTYRCVSVLTVLHQDWLNGKEYKCKVS |
| | | | NKALPAPIEKTISKAKGQPREPQVYTLPPSREEMTKNQVSLTCLVKGFYPSDIAVEWE |
| | | | SNGQPENNYKTTPPVLDSDGSFFLYSKLTVDKSRWQQGNVFSCSVMHEALHNHYTQKS |
| | | | LSLSPGK |
| 2175 | CD33 E11 CC | artificial | QVQLVQSGAEVKKPGESVKVSCKASGYTFTNYGMNWVKQAPGQCLEWMGWINTYTGEP |
| | x I2K x | | TYADKFQGRVTMTTDTSTSTAYMEIRNLGGDDTAVYYCARWSWSDGYYVYFDYWGQGT |
| | scFc-Full | | SVTVSSGGGGSGGGGSGGGGSDIVMTQSPDSLTVSLGERTTINCKSSQSVLDSSTNKN |
| | Sequence | | SLAWYQQKPGQPPKLLLSWASTRESGIPDRFSGSGSGTDFTLTIDSPQPEDSATYYCQ |
| | | | QSAHFPITFGCGTRLEIKSGGGGSEVQLVESGGGLVQPGGSLKLSCAASGFTFNKYAM |
| | | | NWVRQAPGKGLEWVARIRSKYNNYATYYAEAVKGRFTISRDDSKNTVYLQMNNLKTED |
| | | | TAVYYCVRNENIGTSYISYWAYWGQGTLVTVSSGGGGSGGGGSGGGGSQTVVTQEPSL |
| | | | TVSPGGTVTITCGSSTGAVTSGNYPNWVQQKPGQAPRGLIGGTKFLAPGTPARFSGSL |
| | | | SGGKAALTLSGVQPEDEAEYYCVLWYSNRWVFGSGTKLTVLGGGGDKTHTCPPCPAPE |
| | | | LLGGPSVFLFPPKPKDTLMISRTPEVTCVVVDVSHEDPEVKENWYVDGVEVHNAKTKP |
| | | | CEEQYGSTYRCVSVLTVLHQDWLNGKEYKCKVSNKALPAPIEKTISKAKGQPREPQVY |
| | | | TLPPSREEMTKNQVSLTCLVKGFYPSDIAVEWESNGQPENNYKTTPPVLDSDGSFFLY |
| | | | SKLTVDKSRWQQGNVFSCSVMHEALHNHYTQKSLSLSPGKGGGGSGGGGSGGGGSGGG |
| | | | GSGGGGSGGGGSDKTHTCPPCPAPELLGGPSVFLFPPKPKDTLMISRTPEVTCVVVDV |
| | | | SHEDPEVKFNWYVDGVEVHNAKTKPCEEQYGSTYRCVSVLTVLHQDWLNGKEYKCKVS |
| | | | NKALPAPIEKTISKAKGQPREPQVYTLPPSREEMTKNQVSLTCLVKGFYPSDIAVEWE |
| | | | SNGQPENNYKTTPPVLDSDGSFFLYSKLTVDKSRWQQGNVFSCSVMHEALHNHYTQKS |
| | | | LSLSPGK |
| 2176 | CD33 E11 CC | artificial | QVQLVQSGAEVKKPGESVKVSCKASGYTFTNYGMNWVKQAPGQCLEWMGWINTYTGEP |
| | x I2L x | | TYADKFQGRVTMTTDTSTSTAYMEIRNLGGDDTAVYYCARWSWSDGYYVYFDYWGQGT |
| | scFc-Full | | SVTVSSGGGGSGGGGSGGGGSDIVMTQSPDSLTVSLGERTTINCKSSQSVLDSSTNKN |
| | Sequence | | SLAWYQQKPGQPPKLLLSWASTRESGIPDRFSGSGSGTDFTLTIDSPQPEDSATYYCQ |
| | | | QSAHFPITFGCGTRLEIKSGGGGSEVQLVESGGGLVQPGGSLKLSCAASGFTENKYAM |
| | | | NWVRQAPGKGMEWVARIRSKYNNYATYYADAVKDRFTISRDDSKNTLYLQMNNLKTED |
| | | | TAVYYCVRAGNFGSSYISYFAYWGQGTLVTVSSGGGGSGGGGSGGGGSQTVVTQEPSL |
| | | | TVSPGGTVTITCGSSTGAVTSGNYPNWIQKKPGQAPRGLIGGTKFLAPGTPARFSGSL |
| | | | EGGKAALTLSGVQPEDEAEYYCVLYYSNRWVFGSGTKLTVLGGGGDKTHTCPPCPAPE |
| | | | LLGGPSVFLFPPKPKDTLMISRTPEVTCVVVDVSHEDPEVKENWYVDGVEVHNAKTKP |
| | | | CEEQYGSTYRCVSVLTVLHQDWLNGKEYKCKVSNKALPAPIEKTISKAKGQPREPQVY |
| | | | TLPPSREEMTKNQVSLTCLVKGFYPSDIAVEWESNGQPENNYKTTPPVLDSDGSFFLY |
| | | | SKLTVDKSRWQQGNVFSCSVMHEALHNHYTQKSLSLSPGKGGGGSGGGGSGGGGSGGG |
| | | | GSGGGGSGGGGSDKTHTCPPCPAPELLGGPSVFLFPPKPKDTLMISRTPEVTCVVVDV |
| | | | SHEDPEVKFNWYVDGVEVHNAKTKPCEEQYGSTYRCVSVLTVLHQDWLNGKEYKCKVS |
| | | | NKALPAPIEKTISKAKGQPREPQVYTLPPSREEMTKNQVSLTCLVKGFYPSDIAVEWE |
| | | | SNGQPENNYKTTPPVLDSDGSFFLYSKLTVDKSRWQQGNVFSCSVMHEALHNHYTQKS |
| | | | LSLSPGK |
| 2177 | CD33 E11 CC | artificial | QVQLVQSGAEVKKPGESVKVSCKASGYTFTNYGMNWVKQAPGQCLEWMGWINTYTGEP |
| | x I2M X | | TYADKFQGRVTMTTDTSTSTAYMEIRNLGGDDTAVYYCARWSWSDGYYVYFDYWGQGT |
| | scFc-Full | | SVTVSSGGGGSGGGGSGGGGSDIVMTQSPDSLTVSLGERTTINCKSSQSVLDSSTNKN |
| | Sequence | | SLAWYQQKPGQPPKLLLSWASTRESGIPDRFSGSGSGTDFTLTIDSPQPEDSATYYCQ |
| | | | QSAHFPITFGCGTRLEIKSGGGGSEVQLVESGGGLVQPGGSLKLSCAASGFTENKYAM |
| | | | NWVRQAPGKGMEWVARIRSKYNNYATYYADAVKDRFTISRDDSKNTLYLQMNNLKTED |
| | | | TAVYYCVRAGNFGTSYISYWAYWGQGTLVTVSSGGGGSGGGGSGGGGSQTVVTQEPSL |
| | | | TVSPGGTVTITCGSSTGAVTSGNYPNWVQKKPGQAPRGLIGGTKFLAPGTPARFSGSL |
| | | | SGGKAALTLSGVQPEDEAEYYCVLWYSNRWVFGSGTKLTVLGGGGDKTHTCPPCPAPE |
| | | | LLGGPSVFLFPPKPKDTLMISRTPEVTCVVVDVSHEDPEVKENWYVDGVEVHNAKTKP |
| | | | CEEQYGSTYRCVSVLTVLHQDWLNGKEYKCKVSNKALPAPIEKTISKAKGQPREPQVY |
| | | | TLPPSREEMTKNQVSLTCLVKGFYPSDIAVEWESNGQPENNYKTTPPVLDSDGSFFLY |
| | | | SKLTVDKSRWQQGNVFSCSVMHEALHNHYTQKSLSLSPGKGGGGSGGGGSGGGGSGGG |
| | | | GSGGGGSGGGGSDKTHTCPPCPAPELLGGPSVFLFPPKPKDTLMISRTPEVTCVVVDV |
| | | | SHEDPEVKFNWYVDGVEVHNAKTKPCEEQYGSTYRCVSVLTVLHQDWLNGKEYKCKVS |
| | | | NKALPAPIEKTISKAKGQPREPQVYTLPPSREEMTKNQVSLTCLVKGFYPSDIAVEWE |
| | | | SNGQPENNYKTTPPVLDSDGSFFLYSKLTVDKSRWQQGNVFSCSVMHEALHNHYTQKS |
| | | | LSLSPGK |
| 2178 | CD33 E11 CC | artificial | QVQLVQSGAEVKKPGESVKVSCKASGYTFTNYGMNWVKQAPGQCLEWMGWINTYTGEP |
| | X I2M2 X | | TYADKFQGRVTMTTDTSTSTAYMEIRNLGGDDTAVYYCARWSWSDGYYVYFDYWGQGT |
| | scFc-Full | | SVTVSSGGGGSGGGGSGGGGSDIVMTQSPDSLTVSLGERTTINCKSSQSVLDSSTNKN |
| | Sequence | | SLAWYQQKPGQPPKLLLSWASTRESGIPDRFSGSGSGTDFTLTIDSPQPEDSATYYCQ |
| | | | QSAHFPITFGCGTRLEIKSGGGGSEVQLVESGGGLVQPGGSLKLSCAASGFTFNKYAI |
| | | | NWVREAPGKGLEWVARIRSKYNNYATYYADAVKDRFTISRDDSKNTAYLQMNNLKTED |
| | | | TAVYYCVRNANFGTSYISYFAYWGQGTLVTVSSGGGGSGGGGSGGGGSQTVVTQEPSL |
| | | | TVSPGGTVTLTCGSSTGAVTSGNYPNWVQKKPGQAPRGLIGGTKFLAPGTPARFSGSL |
| | | | LGGKAALTLSGVQPEDEAEYYCVLWYSNRWVFGSGTKLTVLGGGGDKTHTCPPCPAPE |
| | | | LLGGPSVFLFPPKPKDTLMISRTPEVTCVVVDVSHEDPEVKENWYVDGVEVHNAKTKP |
| | | | CEEQYGSTYRCVSVLTVLHQDWLNGKEYKCKVSNKALPAPIEKTISKAKGQPREPQVY |
| | | | TLPPSREEMTKNQVSLTCLVKGFYPSDIAVEWESNGQPENNYKTTPPVLDSDGSFFLY |
| | | | SKLTVDKSRWQQGNVFSCSVMHEALHNHYTQKSLSLSPGKGGGGSGGGGSGGGGSGGG |
| | | | GSGGGGSGGGGSDKTHTCPPCPAPELLGGPSVFLFPPKPKDTLMISRTPEVTCVVVDV |
| | | | SHEDPEVKFNWYVDGVEVHNAKTKPCEEQYGSTYRCVSVLTVLHQDWLNGKEYKCKVS |
| | | | NKALPAPIEKTISKAKGQPREPQVYTLPPSREEMTKNQVSLTCLVKGFYPSDIAVEWE |
| | | | SNGQPENNYKTTPPVLDSDGSFFLYSKLTVDKSRWQQGNVFSCSVMHEALHNHYTQKS |
| | | | LSLSPGK |
| 2179 | CD70 1 C7D | artificial | TYAMS |
| | CC-HCDR1 | | |
| 2180 | CD70 1 C7D | artificial | AISGSGGRTFYAESVEG |
| | CC-HCDR2 | | |
| 2181 | CD70 1 C7D | artificial | HDYSNYPYFDY |
| | CC-HCDR3 | | |
| 2182 | CD70 1 C7D | artificial | RASQSVRSTYLA |
| | CC-LCDR1 | | |
| 2183 | CD70 1 C7D | artificial | GASSRAT |
| | CC-LCDR2 | | |
| 2184 | CD70 1 C7D | artificial | QQYGDLPFT |
| | CC-LCDR3 | | |
| 2185 | CD70 1 C7D | artificial | EVQLLESGGGMVQPGGSLRLSCAASGFTFSTYAMSWVRQAPGKCLEWVSAISGSGGRT |
| | CC-VH | | FYAESVEGRFTISRDNSKNTLYLQMNSLRAEDTAVYYCAKHDYSNYPYFDYWGQGTLV |
| | | | TVSS |
| 2186 | CD70 1 C7D | artificial | EIVLTQSPGTLSLSPGERATLSCRASQSVRSTYLAWYQQKPGQAPRLLIYGASSRATG |
| | CC-VL | | IPDRESGSGSGTDFTLTISRLEPEDFAVYSCQQYGDLPFTFGCGTKLEIK |
| 2187 | CD70 1 C7D | artificial | EVQLLESGGGMVQPGGSLRLSCAASGFTFSTYAMSWVRQAPGKCLEWVSAISGSGGRT |
| | CC x I2C x | | FYAESVEGRETISRDNSKNTLYLQMNSLRAEDTAVYYCAKHDYSNYPYFDYWGQGTLV |
| | ScFc-Full | | TVSSGGGGSGGGGSGGGGSEIVLTQSPGTLSLSPGERATLSCRASQSVRSTYLAWYQQ |
| | Sequence | | KPGQAPRLLIYGASSRATGIPDRFSGSGSGTDFTLTISRLEPEDFAVYSCQQYGDLPF |
| | | | TFGCGTKLEIKSGGGGSEVQLVESGGGLVQPGGSLKLSCAASGFTFNKYAMNWVRQAP |
| | | | GKGLEWVARIRSKYNNYATYYADSVKDRFTISRDDSKNTAYLQMNNLKTEDTAVYYCV |
| | | | RHGNFGNSYISYWAYWGQGTLVTVSSGGGGSGGGGSGGGGSQTVVTQEPSLTVSPGGT |
| | | | VTLTCGSSTGAVTSGNYPNWVQQKPGQAPRGLIGGTKFLAPGTPARFSGSLLGGKAAL |
| | | | TLSGVQPEDEAEYYCVLWYSNRWVFGGGTKLTVLGGGGDKTHTCPPCPAPELLGGPSV |
| | | | FLFPPKPKDTLMISRTPEVTCVVVDVSHEDPEVKENWYVDGVEVHNAKTKPCEEQYGS |
| | | | TYRCVSVLTVLHQDWLNGKEYKCKVSNKALPAPIEKTISKAKGQPREPQVYTLPPSRE |
| | | | EMTKNQVSLTCLVKGFYPSDIAVEWESNGQPENNYKTTPPVLDSDGSFFLYSKLTVDK |
| | | | SRWQQGNVFSCSVMHEALHNHYTQKSLSLSPGKGGGGSGGGGSGGGGSGGGGSGGGGS |
| | | | GGGGSDKTHTCPPCPAPELLGGPSVFLFPPKPKDTLMISRTPEVTCVVVDVSHEDPEV |
| | | | KFNWYVDGVEVHNAKTKPCEEQYGSTYRCVSVLTVLHQDWLNGKEYKCKVSNKALPAP |
| | | | IEKTISKAKGQPREPQVYTLPPSREEMTKNQVSLTCLVKGFYPSDIAVEWESNGQPEN |
| | | | NYKTTPPVLDSDGSFFLYSKLTVDKSRWQQGNVFSCSVMHEALHNHYTQKSLSLSPGK |
| 2188 | CD70 1 C7D | artificial | EVQLLESGGGMVQPGGSLRLSCAASGFTFSTYAMSWVRQAPGKCLEWVSAISGSGGRT |
| | CC x I2E X | | FYAESVEGRFTISRDNSKNTLYLQMNSLRAEDTAVYYCAKHDYSNYPYFDYWGQGTLV |
| | scFc-Full | | TVSSGGGGSGGGGSGGGGSEIVLTQSPGTLSLSPGERATLSCRASQSVRSTYLAWYQQ |
| | Sequence | | KPGQAPRLLIYGASSRATGIPDRFSGSGSGTDFTLTISRLEPEDFAVYSCQQYGDLPF |
| | | | TFGCGTKLEIKSGGGGSEVQLVESGGGLVQPGGSLKLSCAASGFTENKYAINWVRQAP |
| | | | GKGLEWVARIRSKYNNYATYYADAVKDRFTISRDDSKNTVYLQMNNLKTEDTAVYYCA |
| | | | RAGNFGSSYISYWAYWGQGTLVTVSSGGGGSGGGGSGGGGSQTVVTQEPSLTVSPGGT |
| | | | VTITCGSSTGAVTSGNYPNWVQKKPGQAPRGLIGGTKFLAPGTPARFSGSLSGGKAAL |
| | | | TLSGVQPEDEAEYYCVLWYSNRWVFGSGTKLTVLGGGGDKTHTCPPCPAPELLGGPSV |
| | | | FLFPPKPKDTLMISRTPEVTCVVVDVSHEDPEVKFNWYVDGVEVHNAKTKPCEEQYGS |
| | | | TYRCVSVLTVLHQDWLNGKEYKCKVSNKALPAPIEKTISKAKGQPREPQVYTLPPSRE |
| | | | EMTKNQVSLTCLVKGFYPSDIAVEWESNGQPENNYKTTPPVLDSDGSFFLYSKLTVDK |
| | | | SRWQQGNVFSCSVMHEALHNHYTQKSLSLSPGKGGGGSGGGGSGGGGSGGGGSGGGGS |
| | | | GGGGSDKTHTCPPCPAPELLGGPSVFLFPPKPKDTLMISRTPEVTCVVVDVSHEDPEV |
| | | | KFNWYVDGVEVHNAKTKPCEEQYGSTYRCVSVLTVLHQDWLNGKEYKCKVSNKALPAP |
| | | | IEKTISKAKGQPREPQVYTLPPSREEMTKNQVSLTCLVKGFYPSDIAVEWESNGQPEN |
| | | | NYKTTPPVLDSDGSFFLYSKLTVDKSRWQQGNVFSCSVMHEALHNHYTQKSLSLSPGK |
| 2189 | CD70 1 C7D | artificial | EVQLLESGGGMVQPGGSLRLSCAASGFTFSTYAMSWVRQAPGKCLEWVSAISGSGGRT |
| | CC x I2K X | | FYAESVEGRFTISRDNSKNTLYLQMNSLRAEDTAVYYCAKHDYSNYPYFDYWGQGTLV |
| | scFc-Full | | TVSSGGGGSGGGGSGGGGSEIVLTQSPGTLSLSPGERATLSCRASQSVRSTYLAWYQQ |
| | Sequence | | KPGQAPRLLIYGASSRATGIPDRESGSGSGTDFTLTISRLEPEDFAVYSCQQYGDLPF |
| | | | TFGCGTKLEIKSGGGGSEVQLVESGGGLVQPGGSLKLSCAASGFTFNKYAMNWVRQAP |
| | | | GKGLEWVARIRSKYNNYATYYAEAVKGRFTISRDDSKNTVYLQMNNLKTEDTAVYYCV |
| | | | RNENIGTSYISYWAYWGQGTLVTVSSGGGGSGGGGSGGGGSQTVVTQEPSLTVSPGGT |
| | | | VTITCGSSTGAVTSGNYPNWVQQKPGQAPRGLIGGTKFLAPGTPARFSGSLSGGKAAL |
| | | | TLSGVQPEDEAEYYCVLWYSNRWVFGSGTKLTVLGGGGDKTHTCPPCPAPELLGGPSV |
| | | | FLFPPKPKDTLMISRTPEVTCVVVDVSHEDPEVKENWYVDGVEVHNAKTKPCEEQYGS |
| | | | TYRCVSVLTVLHQDWLNGKEYKCKVSNKALPAPIEKTISKAKGQPREPQVYTLPPSRE |
| | | | EMTKNQVSLTCLVKGFYPSDIAVEWESNGQPENNYKTTPPVLDSDGSFFLYSKLTVDK |
| | | | SRWQQGNVFSCSVMHEALHNHYTQKSLSLSPGKGGGGSGGGGSGGGGSGGGGSGGGGS |
| | | | GGGGSDKTHTCPPCPAPELLGGPSVFLFPPKPKDTLMISRTPEVTCVVVDVSHEDPEV |
| | | | KFNWYVDGVEVHNAKTKPCEEQYGSTYRCVSVLTVLHQDWLNGKEYKCKVSNKALPAP |
| | | | IEKTISKAKGQPREPQVYTLPPSREEMTKNQVSLTCLVKGFYPSDIAVEWESNGQPEN |
| | | | NYKTTPPVLDSDGSFFLYSKLTVDKSRWQQGNVFSCSVMHEALHNHYTQKSLSLSPGK |
| 2190 | CD70 1 C7D | artificial | EVQLLESGGGMVQPGGSLRLSCAASGFTFSTYAMSWVRQAPGKCLEWVSAISGSGGRT |
| | CC x I2L x | | FYAESVEGRETISRDNSKNTLYLQMNSLRAEDTAVYYCAKHDYSNYPYFDYWGQGTLV |
| | scFc-Full | | TVSSGGGGSGGGGSGGGGSEIVLTQSPGTLSLSPGERATLSCRASQSVRSTYLAWYQQ |
| | Sequence | | KPGQAPRLLIYGASSRATGIPDRFSGSGSGTDFTLTISRLEPEDFAVYSCQQYGDLPF |
| | | | TFGCGTKLEIKSGGGGSEVQLVESGGGLVQPGGSLKLSCAASGFTFNKYAMNWVRQAP |
| | | | GKGMEWVARIRSKYNNYATYYADAVKDRFTISRDDSKNTLYLQMNNLKTEDTAVYYCV |
| | | | RAGNFGSSYISYFAYWGQGTLVTVSSGGGGSGGGGSGGGGSQTVVTQEPSLTVSPGGT |
| | | | VTITCGSSTGAVTSGNYPNWIQKKPGQAPRGLIGGTKFLAPGTPARFSGSLEGGKAAL |
| | | | TLSGVQPEDEAEYYCVLYYSNRWVFGSGTKLTVLGGGGDKTHTCPPCPAPELLGGPSV |
| | | | FLFPPKPKDTLMISRTPEVTCVVVDVSHEDPEVKENWYVDGVEVHNAKTKPCEEQYGS |
| | | | TYRCVSVLTVLHQDWLNGKEYKCKVSNKALPAPIEKTISKAKGQPREPQVYTLPPSRE |
| | | | EMTKNQVSLTCLVKGFYPSDIAVEWESNGQPENNYKTTPPVLDSDGSFFLYSKLTVDK |
| | | | SRWQQGNVFSCSVMHEALHNHYTQKSLSLSPGKGGGGSGGGGSGGGGSGGGGSGGGGS |
| | | | GGGGSDKTHTCPPCPAPELLGGPSVFLFPPKPKDTLMISRTPEVTCVVVDVSHEDPEV |
| | | | KFNWYVDGVEVHNAKTKPCEEQYGSTYRCVSVLTVLHQDWLNGKEYKCKVSNKALPAP |
| | | | IEKTISKAKGQPREPQVYTLPPSREEMTKNQVSLTCLVKGFYPSDIAVEWESNGQPEN |
| | | | NYKTTPPVLDSDGSFFLYSKLTVDKSRWQQGNVFSCSVMHEALHNHYTQKSLSLSPGK |
| 2191 | CD70 1 C7D | artificial | EVQLLESGGGMVQPGGSLRLSCAASGFTFSTYAMSWVRQAPGKCLEWVSAISGSGGRT |
| | CC x I2M x | | FYAESVEGRETISRDNSKNTLYLQMNSLRAEDTAVYYCAKHDYSNYPYFDYWGQGTLV |
| | scFc-Full | | TVSSGGGGSGGGGSGGGGSEIVLTQSPGTLSLSPGERATLSCRASQSVRSTYLAWYQQ |
| | Sequence | | KPGQAPRLLIYGASSRATGIPDRFSGSGSGTDFTLTISRLEPEDFAVYSCQQYGDLPF |
| | | | TFGCGTKLEIKSGGGGSEVQLVESGGGLVQPGGSLKLSCAASGFTFNKYAMNWVRQAP |
| | | | GKGMEWVARIRSKYNNYATYYADAVKDRFTISRDDSKNTLYLQMNNLKTEDTAVYYCV |
| | | | RAGNFGTSYISYWAYWGQGTLVTVSSGGGGSGGGGSGGGGSQTVVTQEPSLTVSPGGT |
| | | | VTITCGSSTGAVTSGNYPNWVQKKPGQAPRGLIGGTKFLAPGTPARFSGSLSGGKAAL |
| | | | TLSGVQPEDEAEYYCVLWYSNRWVFGSGTKLTVLGGGGDKTHTCPPCPAPELLGGPSV |
| | | | FLFPPKPKDTLMISRTPEVTCVVVDVSHEDPEVKENWYVDGVEVHNAKTKPCEEQYGS |
| | | | TYRCVSVLTVLHQDWLNGKEYKCKVSNKALPAPIEKTISKAKGQPREPQVYTLPPSRE |
| | | | EMTKNQVSLTCLVKGFYPSDIAVEWESNGQPENNYKTTPPVLDSDGSFFLYSKLTVDK |
| | | | SRWQQGNVFSCSVMHEALHNHYTQKSLSLSPGKGGGGSGGGGSGGGGSGGGGSGGGGS |
| | | | GGGGSDKTHTCPPCPAPELLGGPSVFLFPPKPKDTLMISRTPEVTCVVVDVSHEDPEV |
| | | | KFNWYVDGVEVHNAKTKPCEEQYGSTYRCVSVLTVLHQDWLNGKEYKCKVSNKALPAP |
| | | | IEKTISKAKGQPREPQVYTLPPSREEMTKNQVSLTCLVKGFYPSDIAVEWESNGQPEN |
| | | | NYKTTPPVLDSDGSFFLYSKLTVDKSRWQQGNVFSCSVMHEALHNHYTQKSLSLSPGK |
| 2192 | CD70 1 C7D | artificial | EVQLLESGGGMVQPGGSLRLSCAASGFTFSTYAMSWVRQAPGKCLEWVSAISGSGGRT |
| | CC x I2M2 X | | FYAESVEGRFTISRDNSKNTLYLQMNSLRAEDTAVYYCAKHDYSNYPYFDYWGQGTLV |
| | scFc-Full | | TVSSGGGGSGGGGSGGGGSEIVLTQSPGTLSLSPGERATLSCRASQSVRSTYLAWYQQ |
| | Sequence | | KPGQAPRLLIYGASSRATGIPDRFSGSGSGTDFTLTISRLEPEDFAVYSCQQYGDLPF |
| | | | TFGCGTKLEIKSGGGGSEVQLVESGGGLVQPGGSLKLSCAASGFTENKYAINWVREAP |
| | | | GKGLEWVARIRSKYNNYATYYADAVKDRFTISRDDSKNTAYLQMNNLKTEDTAVYYCV |
| | | | RNANFGTSYISYFAYWGQGTLVTVSSGGGGSGGGGSGGGGSQTVVTQEPSLTVSPGGT |
| | | | VTLTCGSSTGAVTSGNYPNWVQKKPGQAPRGLIGGTKFLAPGTPARFSGSLLGGKAAL |
| | | | TLSGVQPEDEAEYYCVLWYSNRWVFGSGTKLTVLGGGGDKTHTCPPCPAPELLGGPSV |
| | | | FLFPPKPKDTLMISRTPEVTCVVVDVSHEDPEVKFNWYVDGVEVHNAKTKPCEEQYGS |
| | | | TYRCVSVLTVLHQDWLNGKEYKCKVSNKALPAPIEKTISKAKGQPREPQVYTLPPSRE |
| | | | EMTKNQVSLTCLVKGFYPSDIAVEWESNGQPENNYKTTPPVLDSDGSFFLYSKLTVDK |
| | | | SRWQQGNVFSCSVMHEALHNHYTQKSLSLSPGKGGGGSGGGGSGGGGSGGGGSGGGGS |
| | | | GGGGSDKTHTCPPCPAPELLGGPSVFLFPPKPKDTLMISRTPEVTCVVVDVSHEDPEV |
| | | | KFNWYVDGVEVHNAKTKPCEEQYGSTYRCVSVLTVLHQDWLNGKEYKCKVSNKALPAP |
| | | | IEKTISKAKGQPREPQVYTLPPSREEMTKNQVSLTCLVKGFYPSDIAVEWESNGQPEN |
| | | | NYKTTPPVLDSDGSFFLYSKLTVDKSRWQQGNVFSCSVMHEALHNHYTQKSLSLSPGK |
| 2193 | CH19 | artificial | SYGMH |
| | 2G6.007 CC | | |
| | -HCDR1 | | |
| 2194 | CH19 | artificial | FIWYEGSNKYYAESVKD |
| | 2G6.007 CC | | |
| | -HCDR2 | | |
| 2195 | CH19 | artificial | RAGIIGTIGYYYGMDV |
| | 2G6.007 CC | | |
| | -HCDR3 | | |
| 2196 | CH19 | artificial | SGDRLGEKYTS |
| | 2G6.007 CC | | |
| | -LCDR1 | | |
| 2197 | CH19 | artificial | QDTKRPS |
| | 2G6.007 CC | | |
| | -LCDR2 | | |
| 2198 | CH19 | artificial | QAWESSTVV |
| | 2G6.007 CC | | |
| | -LCDR3 | | |
| 2199 | CH19 | artificial | QVQLVESGGGVVQPGGSLRLSCAASGFTFSSYGMHWVRQAPGKCLEWVAFIWYEGSNK |
| | 2G6.007 CC | | YYAESVKDRFTISRDNSKNTLYLQMNSLRAEDTAVYYCARRAGIIGTIGYYYGMDVWG |
| | -VH | | QGTTVTVSS |
| 2200 | CH19 | artificial | SYELTQPPSVSVSPGQTASITCSGDRLGEKYTSWYQQRPGQSPLLVIYQDTKRPSGIP |
| | 2G6.007 CC | | ERFSGSNSGNTATLTISGTQAMDEADYYCQAWESSTVVFGCGTKLTVL |
| | -VL | | |
| 2201 | CH19 | artificial | QVQLVESGGGVVQPGGSLRLSCAASGFTFSSYGMHWVRQAPGKCLEWVAFIWYEGSNK |
| | 2G6.007 CCx | | YYAESVKDRFTISRDNSKNTLYLQMNSLRAEDTAVYYCARRAGIIGTIGYYYGMDVWG |
| | I2C x scFc | | QGTTVTVSSGGGGSGGGGSGGGGSSYELTQPPSVSVSPGQTASITCSGDRLGEKYTSW |
| | -Full | | YQQRPGQSPLLVIYQDTKRPSGIPERFSGSNSGNTATLTISGTQAMDEADYYCQAWES |
| | Sequence | | STVVFGCGTKLTVLSGGGGSEVQLVESGGGLVQPGGSLKLSCAASGFTFNKYAMNWVR |
| | | | QAPGKGLEWVARIRSKYNNYATYYADSVKDRFTISRDDSKNTAYLQMNNLKTEDTAVY |
| | | | YCVRHGNFGNSYISYWAYWGQGTLVTVSSGGGGSGGGGSGGGGSQTVVTQEPSLTVSP |
| | | | GGTVTLTCGSSTGAVTSGNYPNWVQQKPGQAPRGLIGGTKFLAPGTPARFSGSLLGGK |
| | | | AALTLSGVQPEDEAEYYCVLWYSNRWVFGGGTKLTVLGGGGDKTHTCPPCPAPELLGG |
| | | | PSVFLFPPKPKDTLMISRTPEVTCVVVDVSHEDPEVKENWYVDGVEVHNAKTKPCEEQ |
| | | | YGSTYRCVSVLTVLHQDWLNGKEYKCKVSNKALPAPIEKTISKAKGQPREPQVYTLPP |
| | | | SREEMTKNQVSLTCLVKGFYPSDIAVEWESNGQPENNYKTTPPVLDSDGSFFLYSKLT |
| | | | VDKSRWQQGNVFSCSVMHEALHNHYTQKSLSLSPGKGGGGSGGGGSGGGGSGGGGSGG |
| | | | GGSGGGGSDKTHTCPPCPAPELLGGPSVFLFPPKPKDTLMISRTPEVTCVVVDVSHED |
| | | | PEVKFNWYVDGVEVHNAKTKPCEEQYGSTYRCVSVLTVLHQDWLNGKEYKCKVSNKAL |
| | | | PAPIEKTISKAKGQPREPQVYTLPPSREEMTKNQVSLTCLVKGFYPSDIAVEWESNGQ |
| | | | PENNYKTTPPVLDSDGSFFLYSKLTVDKSRWQQGNVFSCSVMHEALHNHYTQKSLSLS |
| | | | PGK |
| 2202 | CH19 | artificial | QVQLVESGGGVVQPGGSLRLSCAASGFTFSSYGMHWVRQAPGKCLEWVAFIWYEGSNK |
| | 2G6.007 CCx | | YYAESVKDRFTISRDNSKNTLYLQMNSLRAEDTAVYYCARRAGIIGTIGYYYGMDVWG |
| | I2C x scFc | | QGTTVTVSSGGGGSGGGGSGGGGSSYELTQPPSVSVSPGQTASITCSGDRLGEKYTSW |
| | delGK- | | YQQRPGQSPLLVIYQDTKRPSGIPERFSGSNSGNTATLTISGTQAMDEADYYCQAWES |
| | Full | | STVVFGCGTKLTVLSGGGGSEVQLVESGGGLVQPGGSLKLSCAASGFTFNKYAMNWVR |
| | Sequence | | QAPGKGLEWVARIRSKYNNYATYYADSVKDRFTISRDDSKNTAYLQMNNLKTEDTAVY |
| | | | YCVRHGNFGNSYISYWAYWGQGTLVTVSSGGGGSGGGGSGGGGSQTVVTQEPSLTVSP |
| | | | GGTVTLTCGSSTGAVTSGNYPNWVQQKPGQAPRGLIGGTKFLAPGTPARFSGSLLGGK |
| | | | AALTLSGVQPEDEAEYYCVLWYSNRWVFGGGTKLTVLGGGGDKTHTCPPCPAPELLGG |
| | | | PSVFLFPPKPKDTLMISRTPEVTCVVVDVSHEDPEVKENWYVDGVEVHNAKTKPCEEQ |
| | | | YGSTYRCVSVLTVLHQDWLNGKEYKCKVSNKALPAPIEKTISKAKGQPREPQVYTLPP |
| | | | SREEMTKNQVSLTCLVKGFYPSDIAVEWESNGQPENNYKTTPPVLDSDGSFFLYSKLT |
| | | | VDKSRWQQGNVFSCSVMHEALHNHYTQKSLSLSPGGGGSGGGGSGGGGSGGGGSGGGG |
| | | | SGGGGSDKTHTCPPCPAPELLGGPSVFLFPPKPKDTLMISRTPEVTCVVVDVSHEDPE |
| | | | VKFNWYVDGVEVHNAKTKPCEEQYGSTYRCVSVLTVLHQDWLNGKEYKCKVSNKALPA |
| | | | PIEKTISKAKGQPREPQVYTLPPSREEMTKNQVSLTCLVKGFYPSDIAVEWESNGQPE |
| | | | NNYKTTPPVLDSDGSFFLYSKLTVDKSRWQQGNVFSCSVMHEALHNHYTQKSLSLSPG |
| | | | K |
| 2203 | CH19 | artificial | QVQLVESGGGVVQPGGSLRLSCAASGFTFSSYGMHWVRQAPGKCLEWVAFIWYEGSNK |
| | 2G6.007 CCx | | YYAESVKDRFTISRDNSKNTLYLQMNSLRAEDTAVYYCARRAGIIGTIGYYYGMDVWG |
| | I2E x scFc | | QGTTVTVSSGGGGSGGGGSGGGGSSYELTQPPSVSVSPGQTASITCSGDRLGEKYTSW |
| | -Full | | YQQRPGQSPLLVIYQDTKRPSGIPERFSGSNSGNTATLTISGTQAMDEADYYCQAWES |
| | Sequence | | STVVFGCGTKLTVLSGGGGSEVQLVESGGGLVQPGGSLKLSCAASGFTFNKYAINWVR |
| | | | QAPGKGLEWVARIRSKYNNYATYYADAVKDRFTISRDDSKNTVYLQMNNLKTEDTAVY |
| | | | YCARAGNFGSSYISYWAYWGQGTLVTVSSGGGGSGGGGSGGGGSQTVVTQEPSLTVSP |
| | | | GGTVTITCGSSTGAVTSGNYPNWVQKKPGQAPRGLIGGTKFLAPGTPARFSGSLSGGK |
| | | | AALTLSGVQPEDEAEYYCVLWYSNRWVFGSGTKLTVLGGGGDKTHTCPPCPAPELLGG |
| | | | PSVFLFPPKPKDTLMISRTPEVTCVVVDVSHEDPEVKENWYVDGVEVHNAKTKPCEEQ |
| | | | YGSTYRCVSVLTVLHQDWLNGKEYKCKVSNKALPAPIEKTISKAKGQPREPQVYTLPP |
| | | | SREEMTKNQVSLTCLVKGFYPSDIAVEWESNGQPENNYKTTPPVLDSDGSFFLYSKLT |
| | | | VDKSRWQQGNVFSCSVMHEALHNHYTQKSLSLSPGKGGGGSGGGGSGGGGSGGGGSGG |
| | | | GGSGGGGSDKTHTCPPCPAPELLGGPSVFLFPPKPKDTLMISRTPEVTCVVVDVSHED |
| | | | PEVKFNWYVDGVEVHNAKTKPCEEQYGSTYRCVSVLTVLHQDWLNGKEYKCKVSNKAL |
| | | | PAPIEKTISKAKGQPREPQVYTLPPSREEMTKNQVSLTCLVKGFYPSDIAVEWESNGQ |
| | | | PENNYKTTPPVLDSDGSFFLYSKLTVDKSRWQQGNVFSCSVMHEALHNHYTQKSLSLS |
| | | | PGK |
| 2204 | CH19 | artificial | QVQLVESGGGVVQPGGSLRLSCAASGFTFSSYGMHWVRQAPGKCLEWVAFIWYEGSNK |
| | 2G6.007 CCx | | YYAESVKDRFTISRDNSKNTLYLQMNSLRAEDTAVYYCARRAGIIGTIGYYYGMDVWG |
| | I2E x scFc | | QGTTVTVSSGGGGSGGGGSGGGGSSYELTQPPSVSVSPGQTASITCSGDRLGEKYTSW |
| | delGK- | | YQQRPGQSPLLVIYQDTKRPSGIPERFSGSNSGNTATLTISGTQAMDEADYYCQAWES |
| | Full | | STVVFGCGTKLTVLSGGGGSEVQLVESGGGLVQPGGSLKLSCAASGFTFNKYAINWVR |
| | Sequence | | QAPGKGLEWVARIRSKYNNYATYYADAVKDRFTISRDDSKNTVYLQMNNLKTEDTAVY |
| | | | YCARAGNFGSSYISYWAYWGQGTLVTVSSGGGGSGGGGSGGGGSQTVVTQEPSLTVSP |
| | | | GGTVTITCGSSTGAVTSGNYPNWVQKKPGQAPRGLIGGTKFLAPGTPARFSGSLSGGK |
| | | | AALTLSGVQPEDEAEYYCVLWYSNRWVFGSGTKLTVLGGGGDKTHTCPPCPAPELLGG |
| | | | PSVFLFPPKPKDTLMISRTPEVTCVVVDVSHEDPEVKENWYVDGVEVHNAKTKPCEEQ |
| | | | YGSTYRCVSVLTVLHQDWLNGKEYKCKVSNKALPAPIEKTISKAKGQPREPQVYTLPP |
| | | | SREEMTKNQVSLTCLVKGFYPSDIAVEWESNGQPENNYKTTPPVLDSDGSFFLYSKLT |
| | | | VDKSRWQQGNVFSCSVMHEALHNHYTQKSLSLSPGGGGSGGGGSGGGGSGGGGSGGGG |
| | | | SGGGGSDKTHTCPPCPAPELLGGPSVFLFPPKPKDTLMISRTPEVTCVVVDVSHEDPE |
| | | | VKFNWYVDGVEVHNAKTKPCEEQYGSTYRCVSVLTVLHQDWLNGKEYKCKVSNKALPA |
| | | | PIEKTISKAKGQPREPQVYTLPPSREEMTKNQVSLTCLVKGFYPSDIAVEWESNGQPE |
| | | | NNYKTTPPVLDSDGSFFLYSKLTVDKSRWQQGNVFSCSVMHEALHNHYTQKSLSLSPG |
| | | | K |
| 2205 | CH19 | artificial | QVQLVESGGGVVQPGGSLRLSCAASGFTFSSYGMHWVRQAPGKCLEWVAFIWYEGSNK |
| | 2G6.007 CCx | | YYAESVKDRFTISRDNSKNTLYLQMNSLRAEDTAVYYCARRAGIIGTIGYYYGMDVWG |
| | I2K x scFc | | QGTTVTVSSGGGGSGGGGSGGGGSSYELTQPPSVSVSPGQTASITCSGDRLGEKYTSW |
| | -Full | | YQQRPGQSPLLVIYQDTKRPSGIPERFSGSNSGNTATLTISGTQAMDEADYYCQAWES |
| | Sequence | | STVVFGCGTKLTVLSGGGGSEVQLVESGGGLVQPGGSLKLSCAASGFTENKYAMNWVR |
| | | | QAPGKGLEWVARIRSKYNNYATYYAEAVKGRFTISRDDSKNTVYLQMNNLKTEDTAVY |
| | | | YCVRNENIGTSYISYWAYWGQGTLVTVSSGGGGSGGGGSGGGGSQTVVTQEPSLTVSP |
| | | | GGTVTITCGSSTGAVTSGNYPNWVQQKPGQAPRGLIGGTKFLAPGTPARFSGSLSGGK |
| | | | AALTLSGVQPEDEAEYYCVLWYSNRWVFGSGTKLTVLGGGGDKTHTCPPCPAPELLGG |
| | | | PSVFLFPPKPKDTLMISRTPEVTCVVVDVSHEDPEVKENWYVDGVEVHNAKTKPCEEQ |
| | | | YGSTYRCVSVLTVLHQDWLNGKEYKCKVSNKALPAPIEKTISKAKGQPREPQVYTLPP |
| | | | SREEMTKNQVSLTCLVKGFYPSDIAVEWESNGQPENNYKTTPPVLDSDGSFFLYSKLT |
| | | | VDKSRWQQGNVFSCSVMHEALHNHYTQKSLSLSPGKGGGGSGGGGSGGGGSGGGGSGG |
| | | | GGSGGGGSDKTHTCPPCPAPELLGGPSVFLFPPKPKDTLMISRTPEVTCVVVDVSHED |
| | | | PEVKFNWYVDGVEVHNAKTKPCEEQYGSTYRCVSVLTVLHQDWLNGKEYKCKVSNKAL |
| | | | PAPIEKTISKAKGQPREPQVYTLPPSREEMTKNQVSLTCLVKGFYPSDIAVEWESNGQ |
| | | | PENNYKTTPPVLDSDGSFFLYSKLTVDKSRWQQGNVFSCSVMHEALHNHYTQKSLSLS |
| | | | PGK |
| 2206 | CH19 | artificial | QVQLVESGGGVVQPGGSLRLSCAASGFTFSSYGMHWVRQAPGKCLEWVAFIWYEGSNK |
| | 2G6.007 CCx | | YYAESVKDRFTISRDNSKNTLYLQMNSLRAEDTAVYYCARRAGIIGTIGYYYGMDVWG |
| | I2K x scFc | | QGTTVTVSSGGGGSGGGGSGGGGSSYELTQPPSVSVSPGQTASITCSGDRLGEKYTSW |
| | delGK- | | YQQRPGQSPLLVIYQDTKRPSGIPERFSGSNSGNTATLTISGTQAMDEADYYCQAWES |
| | Full | | STVVFGCGTKLTVLSGGGGSEVQLVESGGGLVQPGGSLKLSCAASGFTENKYAMNWVR |
| | Sequence | | QAPGKGLEWVARIRSKYNNYATYYAEAVKGRFTISRDDSKNTVYLQMNNLKTEDTAVY |
| | | | YCVRNENIGTSYISYWAYWGQGTLVTVSSGGGGSGGGGSGGGGSQTVVTQEPSLTVSP |
| | | | GGTVTITCGSSTGAVTSGNYPNWVQQKPGQAPRGLIGGTKFLAPGTPARFSGSLSGGK |
| | | | AALTLSGVQPEDEAEYYCVLWYSNRWVFGSGTKLTVLGGGGDKTHTCPPCPAPELLGG |
| | | | PSVFLFPPKPKDTLMISRTPEVTCVVVDVSHEDPEVKENWYVDGVEVHNAKTKPCEEQ |
| | | | YGSTYRCVSVLTVLHQDWLNGKEYKCKVSNKALPAPIEKTISKAKGQPREPQVYTLPP |
| | | | SREEMTKNQVSLTCLVKGFYPSDIAVEWESNGQPENNYKTTPPVLDSDGSFFLYSKLT |
| | | | VDKSRWQQGNVFSCSVMHEALHNHYTQKSLSLSPGGGGSGGGGSGGGGSGGGGSGGGG |
| | | | SGGGGSDKTHTCPPCPAPELLGGPSVFLFPPKPKDTLMISRTPEVTCVVVDVSHEDPE |
| | | | VKFNWYVDGVEVHNAKTKPCEEQYGSTYRCVSVLTVLHQDWLNGKEYKCKVSNKALPA |
| | | | PIEKTISKAKGQPREPQVYTLPPSREEMTKNQVSLTCLVKGFYPSDIAVEWESNGQPE |
| | | | NNYKTTPPVLDSDGSFFLYSKLTVDKSRWQQGNVFSCSVMHEALHNHYTQKSLSLSPG |
| | | | K |
| 2207 | CH19 | artificial | QVQLVESGGGVVQPGGSLRLSCAASGFTFSSYGMHWVRQAPGKCLEWVAFIWYEGSNK |
| | 2G6.007 CCx | | YYAESVKDRFTISRDNSKNTLYLQMNSLRAEDTAVYYCARRAGIIGTIGYYYGMDVWG |
| | I2L x scFc | | QGTTVTVSSGGGGSGGGGSGGGGSSYELTQPPSVSVSPGQTASITCSGDRLGEKYTSW |
| | -Full | | YQQRPGQSPLLVIYQDTKRPSGIPERFSGSNSGNTATLTISGTQAMDEADYYCQAWES |
| | Sequence | | STVVFGCGTKLTVLSGGGGSEVQLVESGGGLVQPGGSLKLSCAASGFTFNKYAMNWVR |
| | | | QAPGKGMEWVARIRSKYNNYATYYADAVKDRFTISRDDSKNTLYLQMNNLKTEDTAVY |
| | | | YCVRAGNFGSSYISYFAYWGQGTLVTVSSGGGGSGGGGSGGGGSQTVVTQEPSLTVSP |
| | | | GGTVTITCGSSTGAVTSGNYPNWIQKKPGQAPRGLIGGTKFLAPGTPARFSGSLEGGK |
| | | | AALTLSGVQPEDEAEYYCVLYYSNRWVFGSGTKLTVLGGGGDKTHTCPPCPAPELLGG |
| | | | PSVFLFPPKPKDTLMISRTPEVTCVVVDVSHEDPEVKENWYVDGVEVHNAKTKPCEEQ |
| | | | YGSTYRCVSVLTVLHQDWLNGKEYKCKVSNKALPAPIEKTISKAKGQPREPQVYTLPP |
| | | | SREEMTKNQVSLTCLVKGFYPSDIAVEWESNGQPENNYKTTPPVLDSDGSFFLYSKLT |
| | | | VDKSRWQQGNVFSCSVMHEALHNHYTQKSLSLSPGKGGGGSGGGGSGGGGSGGGGSGG |
| | | | GGSGGGGSDKTHTCPPCPAPELLGGPSVFLFPPKPKDTLMISRTPEVTCVVVDVSHED |
| | | | PEVKFNWYVDGVEVHNAKTKPCEEQYGSTYRCVSVLTVLHQDWLNGKEYKCKVSNKAL |
| | | | PAPIEKTISKAKGQPREPQVYTLPPSREEMTKNQVSLTCLVKGFYPSDIAVEWESNGQ |
| | | | PENNYKTTPPVLDSDGSFFLYSKLTVDKSRWQQGNVFSCSVMHEALHNHYTQKSLSLS |
| | | | PGK |
| 2208 | CH19 | artificial | QVQLVESGGGVVQPGGSLRLSCAASGFTFSSYGMHWVRQAPGKCLEWVAFIWYEGSNK |
| | 2G6.007 CCx | | YYAESVKDRFTISRDNSKNTLYLQMNSLRAEDTAVYYCARRAGIIGTIGYYYGMDVWG |
| | I2L x scFc | | QGTTVTVSSGGGGSGGGGSGGGGSSYELTQPPSVSVSPGQTASITCSGDRLGEKYTSW |
| | delGK- | | YQQRPGQSPLLVIYQDTKRPSGIPERFSGSNSGNTATLTISGTQAMDEADYYCQAWES |
| | Full | | STVVFGCGTKLTVLSGGGGSEVQLVESGGGLVQPGGSLKLSCAASGFTENKYAMNWVR |
| | Sequence | | QAPGKGMEWVARIRSKYNNYATYYADAVKDRFTISRDDSKNTLYLQMNNLKTEDTAVY |
| | | | YCVRAGNFGSSYISYFAYWGQGTLVTVSSGGGGSGGGGSGGGGSQTVVTQEPSLTVSP |
| | | | GGTVTITCGSSTGAVTSGNYPNWIQKKPGQAPRGLIGGTKFLAPGTPARFSGSLEGGK |
| | | | AALTLSGVQPEDEAEYYCVLYYSNRWVFGSGTKLTVLGGGGDKTHTCPPCPAPELLGG |
| | | | PSVFLFPPKPKDTLMISRTPEVTCVVVDVSHEDPEVKFNWYVDGVEVHNAKTKPCEEQ |
| | | | YGSTYRCVSVLTVLHQDWLNGKEYKCKVSNKALPAPIEKTISKAKGQPREPQVYTLPP |
| | | | SREEMTKNQVSLTCLVKGFYPSDIAVEWESNGQPENNYKTTPPVLDSDGSFFLYSKLT |
| | | | VDKSRWQQGNVFSCSVMHEALHNHYTQKSLSLSPGGGGSGGGGSGGGGSGGGGSGGGG |
| | | | SGGGGSDKTHTCPPCPAPELLGGPSVFLFPPKPKDTLMISRTPEVTCVVVDVSHEDPE |
| | | | VKFNWYVDGVEVHNAKTKPCEEQYGSTYRCVSVLTVLHQDWLNGKEYKCKVSNKALPA |
| | | | PIEKTISKAKGQPREPQVYTLPPSREEMTKNQVSLTCLVKGFYPSDIAVEWESNGQPE |
| | | | NNYKTTPPVLDSDGSFFLYSKLTVDKSRWQQGNVFSCSVMHEALHNHYTQKSLSLSPG |
| | | | K |
| 2209 | CH19 | artificial | QVQLVESGGGVVQPGGSLRLSCAASGFTFSSYGMHWVRQAPGKCLEWVAFIWYEGSNK |
| | 2G6.007 CCx | | YYAESVKDRFTISRDNSKNTLYLQMNSLRAEDTAVYYCARRAGIIGTIGYYYGMDVWG |
| | I2M x scFc | | QGTTVTVSSGGGGSGGGGSGGGGSSYELTQPPSVSVSPGQTASITCSGDRLGEKYTSW |
| | -Full | | YQQRPGQSPLLVIYQDTKRPSGIPERFSGSNSGNTATLTISGTQAMDEADYYCQAWES |
| | Sequence | | STVVFGCGTKLTVLSGGGGSEVQLVESGGGLVQPGGSLKLSCAASGFTENKYAMNWVR |
| | | | QAPGKGMEWVARIRSKYNNYATYYADAVKDRFTISRDDSKNTLYLQMNNLKTEDTAVY |
| | | | YCVRAGNFGTSYISYWAYWGQGTLVTVSSGGGGSGGGGSGGGGSQTVVTQEPSLTVSP |
| | | | GGTVTITCGSSTGAVTSGNYPNWVQKKPGQAPRGLIGGTKFLAPGTPARFSGSLSGGK |
| | | | AALTLSGVQPEDEAEYYCVLWYSNRWVFGSGTKLTVLGGGGDKTHTCPPCPAPELLGG |
| | | | PSVFLFPPKPKDTLMISRTPEVTCVVVDVSHEDPEVKENWYVDGVEVHNAKTKPCEEQ |
| | | | YGSTYRCVSVLTVLHQDWLNGKEYKCKVSNKALPAPIEKTISKAKGQPREPQVYTLPP |
| | | | SREEMTKNQVSLTCLVKGFYPSDIAVEWESNGQPENNYKTTPPVLDSDGSFFLYSKLT |
| | | | VDKSRWQQGNVFSCSVMHEALHNHYTQKSLSLSPGKGGGGSGGGGSGGGGSGGGGSGG |
| | | | GGSGGGGSDKTHTCPPCPAPELLGGPSVFLFPPKPKDTLMISRTPEVTCVVVDVSHED |
| | | | PEVKFNWYVDGVEVHNAKTKPCEEQYGSTYRCVSVLTVLHQDWLNGKEYKCKVSNKAL |
| | | | PAPIEKTISKAKGQPREPQVYTLPPSREEMTKNQVSLTCLVKGFYPSDIAVEWESNGQ |
| | | | PENNYKTTPPVLDSDGSFFLYSKLTVDKSRWQQGNVFSCSVMHEALHNHYTQKSLSLS |
| | | | PGK |
| 2210 | CH19 | artificial | QVQLVESGGGVVQPGGSLRLSCAASGFTFSSYGMHWVRQAPGKCLEWVAFIWYEGSNK |
| | 2G6.007 CCx | | YYAESVKDRFTISRDNSKNTLYLQMNSLRAEDTAVYYCARRAGIIGTIGYYYGMDVWG |
| | I2M x scFc | | QGTTVTVSSGGGGSGGGGSGGGGSSYELTQPPSVSVSPGQTASITCSGDRLGEKYTSW |
| | delGK- | | YQQRPGQSPLLVIYQDTKRPSGIPERFSGSNSGNTATLTISGTQAMDEADYYCQAWES |
| | Full | | STVVFGCGTKLTVLSGGGGSEVQLVESGGGLVQPGGSLKLSCAASGFTFNKYAMNWVR |
| | Sequence | | QAPGKGMEWVARIRSKYNNYATYYADAVKDRFTISRDDSKNTLYLQMNNLKTEDTAVY |
| | | | YCVRAGNFGTSYISYWAYWGQGTLVTVSSGGGGSGGGGSGGGGSQTVVTQEPSLTVSP |
| | | | GGTVTITCGSSTGAVTSGNYPNWVQKKPGQAPRGLIGGTKFLAPGTPARFSGSLSGGK |
| | | | AALTLSGVQPEDEAEYYCVLWYSNRWVFGSGTKLTVLGGGGDKTHTCPPCPAPELLGG |
| | | | PSVFLFPPKPKDTLMISRTPEVTCVVVDVSHEDPEVKENWYVDGVEVHNAKTKPCEEQ |
| | | | YGSTYRCVSVLTVLHQDWLNGKEYKCKVSNKALPAPIEKTISKAKGQPREPQVYTLPP |
| | | | SREEMTKNQVSLTCLVKGFYPSDIAVEWESNGQPENNYKTTPPVLDSDGSFFLYSKLT |
| | | | VDKSRWQQGNVFSCSVMHEALHNHYTQKSLSLSPGGGGSGGGGSGGGGSGGGGSGGGG |
| | | | SGGGGSDKTHTCPPCPAPELLGGPSVFLFPPKPKDTLMISRTPEVTCVVVDVSHEDPE |
| | | | VKFNWYVDGVEVHNAKTKPCEEQYGSTYRCVSVLTVLHQDWLNGKEYKCKVSNKALPA |
| | | | PIEKTISKAKGQPREPQVYTLPPSREEMTKNQVSLTCLVKGFYPSDIAVEWESNGQPE |
| | | | NNYKTTPPVLDSDGSFFLYSKLTVDKSRWQQGNVFSCSVMHEALHNHYTQKSLSLSPG |
| | | | K |
| 2211 | CH19 | artificial | QVQLVESGGGVVQPGGSLRLSCAASGFTFSSYGMHWVRQAPGKCLEWVAFIWYEGSNK |
| | 2G6.007 CCx | | YYAESVKDRFTISRDNSKNTLYLQMNSLRAEDTAVYYCARRAGIIGTIGYYYGMDVWG |
| | I2M2 x scFc | | QGTTVTVSSGGGGSGGGGSGGGGSSYELTQPPSVSVSPGQTASITCSGDRLGEKYTSW |
| | -Full | | YQQRPGQSPLLVIYQDTKRPSGIPERFSGSNSGNTATLTISGTQAMDEADYYCQAWES |
| | Sequence | | STVVFGCGTKLTVLSGGGGSEVQLVESGGGLVQPGGSLKLSCAASGFTFNKYAINWVR |
| | | | EAPGKGLEWVARIRSKYNNYATYYADAVKDRFTISRDDSKNTAYLQMNNLKTEDTAVY |
| | | | YCVRNANFGTSYISYFAYWGQGTLVTVSSGGGGSGGGGSGGGGSQTVVTQEPSLTVSP |
| | | | GGTVTLTCGSSTGAVTSGNYPNWVQKKPGQAPRGLIGGTKFLAPGTPARFSGSLLGGK |
| | | | AALTLSGVQPEDEAEYYCVLWYSNRWVFGSGTKLTVLGGGGDKTHTCPPCPAPELLGG |
| | | | PSVFLFPPKPKDTLMISRTPEVTCVVVDVSHEDPEVKFNWYVDGVEVHNAKTKPCEEQ |
| | | | YGSTYRCVSVLTVLHQDWLNGKEYKCKVSNKALPAPIEKTISKAKGQPREPQVYTLPP |
| | | | SREEMTKNQVSLTCLVKGFYPSDIAVEWESNGQPENNYKTTPPVLDSDGSFFLYSKLT |
| | | | VDKSRWQQGNVFSCSVMHEALHNHYTQKSLSLSPGKGGGGSGGGGSGGGGSGGGGSGG |
| | | | GGSGGGGSDKTHTCPPCPAPELLGGPSVFLFPPKPKDTLMISRTPEVTCVVVDVSHED |
| | | | PEVKFNWYVDGVEVHNAKTKPCEEQYGSTYRCVSVLTVLHQDWLNGKEYKCKVSNKAL |
| | | | PAPIEKTISKAKGQPREPQVYTLPPSREEMTKNQVSLTCLVKGFYPSDIAVEWESNGQ |
| | | | PENNYKTTPPVLDSDGSFFLYSKLTVDKSRWQQGNVFSCSVMHEALHNHYTQKSLSLS |
| | | | PGK |
| 2212 | CH19 | artificial | QVQLVESGGGVVQPGGSLRLSCAASGFTFSSYGMHWVRQAPGKCLEWVAFIWYEGSNK |
| | 2G6.007 CCx | | YYAESVKDRFTISRDNSKNTLYLQMNSLRAEDTAVYYCARRAGIIGTIGYYYGMDVWG |
| | I2M2 x scFc | | QGTTVTVSSGGGGSGGGGSGGGGSSYELTQPPSVSVSPGQTASITCSGDRLGEKYTSW |
| | delGK- | | YQQRPGQSPLLVIYQDTKRPSGIPERFSGSNSGNTATLTISGTQAMDEADYYCQAWES |
| | Full | | STVVFGCGTKLTVLSGGGGSEVQLVESGGGLVQPGGSLKLSCAASGFTENKYAINWVR |
| | Sequence | | EAPGKGLEWVARIRSKYNNYATYYADAVKDRFTISRDDSKNTAYLQMNNLKTEDTAVY |
| | | | YCVRNANFGTSYISYFAYWGQGTLVTVSSGGGGSGGGGSGGGGSQTVVTQEPSLTVSP |
| | | | GGTVTLTCGSSTGAVTSGNYPNWVQKKPGQAPRGLIGGTKFLAPGTPARFSGSLLGGK |
| | | | AALTLSGVQPEDEAEYYCVLWYSNRWVFGSGTKLTVLGGGGDKTHTCPPCPAPELLGG |
| | | | PSVFLFPPKPKDTLMISRTPEVTCVVVDVSHEDPEVKENWYVDGVEVHNAKTKPCEEQ |
| | | | YGSTYRCVSVLTVLHQDWLNGKEYKCKVSNKALPAPIEKTISKAKGQPREPQVYTLPP |
| | | | SREEMTKNQVSLTCLVKGFYPSDIAVEWESNGQPENNYKTTPPVLDSDGSFFLYSKLT |
| | | | VDKSRWQQGNVFSCSVMHEALHNHYTQKSLSLSPGGGGSGGGGSGGGGSGGGGSGGGG |
| | | | SGGGGSDKTHTCPPCPAPELLGGPSVFLFPPKPKDTLMISRTPEVTCVVVDVSHEDPE |
| | | | VKFNWYVDGVEVHNAKTKPCEEQYGSTYRCVSVLTVLHQDWLNGKEYKCKVSNKALPA |
| | | | PIEKTISKAKGQPREPQVYTLPPSREEMTKNQVSLTCLVKGFYPSDIAVEWESNGQPE |
| | | | NNYKTTPPVLDSDGSFFLYSKLTVDKSRWQQGNVFSCSVMHEALHNHYTQKSLSLSPG |
| | | | K |
| 2213 | CH3 15-E11 | artificial | NYWMN |
| | CC-HCDR1 | | |
| 2214 | CH3 15-E11 | artificial | NIAYGVKGTNYNQKFQG |
| | CC-HCDR2 | | |
| 2215 | CH3 15-E11 | artificial | RYFYVMDY |
| | CC-HCDR3 | | |
| 2216 | CH3 15-E11 | artificial | RASQDISNYLN |
| | CC-LCDR1 | | |
| 2217 | CH3 15-E11 | artificial | YTSRLHS |
| | CC-LCDR2 | | |
| 2218 | CH3 15-E11 | artificial | VQYAQFPLT |
| | CC-LCDR3 | | |
| 2219 | CH3 15-E11 | artificial | QVQLVQSGAEVKKPGASVKVSCKASGYTFTNYWMNWVRQAPGQCLEWMGNIAYGVKGT |
| | CC-VH | | NYNQKFQGRVTMTVDTSSSTAYMELSRLRSDDTAVYYCATRYFYVMDYWGQGTLVTVS |
| | | | S |
| 2220 | CH3 15-E11 | artificial | DIQMTQSPSSLSASVGDRVTITCRASQDISNYLNWYQQKPGKVPKLLIYYTSRLHSGV |
| | CC-VL | | PSRFSGSGSGTDFTLTISSLQPEDVATYYCVQYAQFPLTFGCGTKVEIK |
| 2221 | CH3 15-E11 | artificial | EIQMTQSPSSLSASVGDRVTITCRASQDISNYLNWYQQKPGKVPKLLIYYTSRLHSGV |
| | CC EI GQ- | | PSRFSGSGSGTDFTLTISSLQPEDVATYYCVQYAQFPLTFGCGTKVEIK |
| | VL | | |
| 2222 | CH3 G8A 6- | artificial | SYPIN |
| | B12-HCDR1 | | |
| 2223 | CH3 G8A 6- | artificial | VIWTGGGTNYASSVKG |
| | B12-HCDR2 | | |
| 2224 | CH3 G8A 6- | artificial | SRGVYDFDGRGAMDY |
| | B12-HCDR3 | | |
| 2225 | CH3 G8A 6- | artificial | KSSQSLLYSSNQKNYFA |
| | B12-LCDR1 | | |
| 2226 | CH3 G8A 6- | artificial | WASTRES |
| | B12-LCDR2 | | |
| 2227 | CH3 G8A 6- | artificial | QQYYSYPYT |
| | B12-LCDR3 | | |
| 2228 | CH3 G8A 6- | artificial | EVQLLESGGGLVQPGGSLRLSCAASGFSFSSYPINWVRQAPGKGLEWVGVIWTGGGTN |
| | B12-VH | | YASSVKGRFTISRDNSKNTVYLQMNSLRAEDTAVYYCAKSRGVYDFDGRGAMDYWGQG |
| | | | TLVTVSS |
| 2229 | CH3 G8A 6- | artificial | DIVMTQSPDSLAVSLGERATINCKSSQSLLYSSNQKNYFAWYQQKPGQPPKLLIYWAS |
| | B12-VL | | TRESGVPDRFSGSGSGTDFTLTISSLQAEDVAVYYCQQYYSYPYTFGQGTKLEIK |
| 2230 | CH3 G8A 6- | artificial | EIVMTQSPDSLAVSLGERATINCKSSQSLLYSSNQKNYFAWYQQKPGQPPKLLIYWAS |
| | B12 EI GQ- | | TRESGVPDRFSGSGSGTDFTLTISSLQAEDVAVYYCQQYYSYPYTFGQGTKLEIK |
| | VL | | |
| 2231 | CH3 R170R | artificial | SYWMH |
| | CC-HCDR1 | | |
| 2232 | CH3 R170R | artificial | KIDPSDDYTNYNQKVKG |
| | CC-HCDR2 | | |
| 2233 | CH3 R170R | artificial | WDYSHFDV |
| | CC-HCDR3 | | |
| 2234 | CH3 R170R | artificial | RASSSVSYMH |
| | CC-LCDR1 | | |
| 2235 | CH3 R170R | artificial | GTSNLVS |
| | CC-LCDR2 | | |
| 2236 | CH3 R170R | artificial | QQWSSYPLT |
| | CC-LCDR3 | | |
| 2237 | CH3 R170R | artificial | EVQLLESGGGLVQPGGSLRLSCAASGFTFSSYWMHWVRQTPGKCLEWVSKIDPSDDYT |
| | CC-VH | | NYNQKVKGRFTISIDKSKNTLYLQMNSLRAEDTAVYYCARWDYSHFDVWGQGTTVTVS |
| | | | S |
| 2238 | CH3 R170R | artificial | EIVMTQSPATLSVSPGERATLTCRASSSVSYMHWYQQKPGQAPRLLIYGTSNLVSGVP |
| | CC-VL | | ARFSGSGSGTEFTLTISSLQSEDFAVYYCQQWSSYPLTFGCGTKVEIK |
| 2239 | CH3 15-E11 | artificial | QVQLVQSGAEVKKPGASVKVSCKASGYTFTNYWMNWVRQAPGQCLEWMGNIAYGVKGT |
| | CC x I2C x | | NYNQKFQGRVTMTVDTSSSTAYMELSRLRSDDTAVYYCATRYFYVMDYWGQGTLVTVS |
| | scFc-Full | | SGGGGSGGGGSGGGGSDIQMTQSPSSLSASVGDRVTITCRASQDISNYLNWYQQKPGK |
| | Sequence | | VPKLLIYYTSRLHSGVPSRFSGSGSGTDFTLTISSLQPEDVATYYCVQYAQFPLTFGC |
| | | | GTKVEIKSGGGGSEVQLVESGGGLVQPGGSLKLSCAASGFTFNKYAMNWVRQAPGKGL |
| | | | EWVARIRSKYNNYATYYADSVKDRFTISRDDSKNTAYLQMNNLKTEDTAVYYCVRHGN |
| | | | FGNSYISYWAYWGQGTLVTVSSGGGGSGGGGSGGGGSQTVVTQEPSLTVSPGGTVTLT |
| | | | CGSSTGAVTSGNYPNWVQQKPGQAPRGLIGGTKFLAPGTPARFSGSLLGGKAALTLSG |
| | | | VQPEDEAEYYCVLWYSNRWVFGGGTKLTVLGGGGDKTHTCPPCPAPELLGGPSVFLFP |
| | | | PKPKDTLMISRTPEVTCVVVDVSHEDPEVKENWYVDGVEVHNAKTKPCEEQYGSTYRC |
| | | | VSVLTVLHQDWLNGKEYKCKVSNKALPAPIEKTISKAKGQPREPQVYTLPPSREEMTK |
| | | | NQVSLTCLVKGFYPSDIAVEWESNGQPENNYKTTPPVLDSDGSFFLYSKLTVDKSRWQ |
| | | | QGNVFSCSVMHEALHNHYTQKSLSLSPGKGGGGSGGGGSGGGGSGGGGSGGGGSGGGG |
| | | | SDKTHTCPPCPAPELLGGPSVFLFPPKPKDTLMISRTPEVTCVVVDVSHEDPEVKENW |
| | | | YVDGVEVHNAKTKPCEEQYGSTYRCVSVLTVLHQDWLNGKEYKCKVSNKALPAPIEKT |
| | | | ISKAKGQPREPQVYTLPPSREEMTKNQVSLTCLVKGFYPSDIAVEWESNGQPENNYKT |
| | | | TPPVLDSDGSFFLYSKLTVDKSRWQQGNVFSCSVMHEALHNHYTQKSLSLSPGK |
| 2240 | CH3 15-E11 | artificial | QVQLVQSGAEVKKPGASVKVSCKASGYTFTNYWMNWVRQAPGQCLEWMGNIAYGVKGT |
| | CC x I2E x | | NYNQKFQGRVTMTVDTSSSTAYMELSRLRSDDTAVYYCATRYFYVMDYWGQGTLVTVS |
| | scFc-Full | | SGGGGSGGGGSGGGGSDIQMTQSPSSLSASVGDRVTITCRASQDISNYLNWYQQKPGK |
| | Sequence | | VPKLLIYYTSRLHSGVPSRFSGSGSGTDFTLTISSLQPEDVATYYCVQYAQFPLTFGC |
| | | | GTKVEIKSGGGGSEVQLVESGGGLVQPGGSLKLSCAASGFTFNKYAINWVRQAPGKGL |
| | | | EWVARIRSKYNNYATYYADAVKDRFTISRDDSKNTVYLQMNNLKTEDTAVYYCARAGN |
| | | | FGSSYISYWAYWGQGTLVTVSSGGGGSGGGGSGGGGSQTVVTQEPSLTVSPGGTVTIT |
| | | | CGSSTGAVTSGNYPNWVQKKPGQAPRGLIGGTKFLAPGTPARFSGSLSGGKAALTLSG |
| | | | VQPEDEAEYYCVLWYSNRWVFGSGTKLTVLGGGGDKTHTCPPCPAPELLGGPSVFLFP |
| | | | PKPKDTLMISRTPEVTCVVVDVSHEDPEVKFNWYVDGVEVHNAKTKPCEEQYGSTYRC |
| | | | VSVLTVLHQDWLNGKEYKCKVSNKALPAPIEKTISKAKGQPREPQVYTLPPSREEMTK |
| | | | NQVSLTCLVKGFYPSDIAVEWESNGQPENNYKTTPPVLDSDGSFFLYSKLTVDKSRWQ |
| | | | QGNVFSCSVMHEALHNHYTQKSLSLSPGKGGGGSGGGGSGGGGSGGGGSGGGGSGGGG |
| | | | SDKTHTCPPCPAPELLGGPSVFLFPPKPKDTLMISRTPEVTCVVVDVSHEDPEVKENW |
| | | | YVDGVEVHNAKTKPCEEQYGSTYRCVSVLTVLHQDWLNGKEYKCKVSNKALPAPIEKT |
| | | | ISKAKGQPREPQVYTLPPSREEMTKNQVSLTCLVKGFYPSDIAVEWESNGQPENNYKT |
| | | | TPPVLDSDGSFFLYSKLTVDKSRWQQGNVFSCSVMHEALHNHYTQKSLSLSPGK |
| 2241 | CH3 15-E11 | artificial | QVQLVQSGAEVKKPGASVKVSCKASGYTFTNYWMNWVRQAPGQCLEWMGNIAYGVKGT |
| | CC x I2K x | | NYNQKFQGRVTMTVDTSSSTAYMELSRLRSDDTAVYYCATRYFYVMDYWGQGTLVTVS |
| | scFc-Full | | SGGGGSGGGGSGGGGSDIQMTQSPSSLSASVGDRVTITCRASQDISNYLNWYQQKPGK |
| | Sequence | | VPKLLIYYTSRLHSGVPSRFSGSGSGTDFTLTISSLQPEDVATYYCVQYAQFPLTFGC |
| | | | GTKVEIKSGGGGSEVQLVESGGGLVQPGGSLKLSCAASGFTFNKYAMNWVRQAPGKGL |
| | | | EWVARIRSKYNNYATYYAEAVKGRFTISRDDSKNTVYLQMNNLKTEDTAVYYCVRNEN |
| | | | IGTSYISYWAYWGQGTLVTVSSGGGGSGGGGSGGGGSQTVVTQEPSLTVSPGGTVTIT |
| | | | CGSSTGAVTSGNYPNWVQQKPGQAPRGLIGGTKFLAPGTPARFSGSLSGGKAALTLSG |
| | | | VQPEDEAEYYCVLWYSNRWVFGSGTKLTVLGGGGDKTHTCPPCPAPELLGGPSVFLFP |
| | | | PKPKDTLMISRTPEVTCVVVDVSHEDPEVKENWYVDGVEVHNAKTKPCEEQYGSTYRC |
| | | | VSVLTVLHQDWLNGKEYKCKVSNKALPAPIEKTISKAKGQPREPQVYTLPPSREEMTK |
| | | | NQVSLTCLVKGFYPSDIAVEWESNGQPENNYKTTPPVLDSDGSFFLYSKLTVDKSRWQ |
| | | | QGNVFSCSVMHEALHNHYTQKSLSLSPGKGGGGSGGGGSGGGGSGGGGSGGGGSGGGG |
| | | | SDKTHTCPPCPAPELLGGPSVFLFPPKPKDTLMISRTPEVTCVVVDVSHEDPEVKENW |
| | | | YVDGVEVHNAKTKPCEEQYGSTYRCVSVLTVLHQDWLNGKEYKCKVSNKALPAPIEKT |
| | | | ISKAKGQPREPQVYTLPPSREEMTKNQVSLTCLVKGFYPSDIAVEWESNGQPENNYKT |
| | | | TPPVLDSDGSFFLYSKLTVDKSRWQQGNVFSCSVMHEALHNHYTQKSLSLSPGK |
| 2242 | CH3 15-E11 | artificial | QVQLVQSGAEVKKPGASVKVSCKASGYTFTNYWMNWVRQAPGQCLEWMGNIAYGVKGT |
| | CC x I2L x | | NYNQKFQGRVTMTVDTSSSTAYMELSRLRSDDTAVYYCATRYFYVMDYWGQGTLVTVS |
| | ScFc-Full | | SGGGGSGGGGSGGGGSDIQMTQSPSSLSASVGDRVTITCRASQDISNYLNWYQQKPGK |
| | Sequence | | VPKLLIYYTSRLHSGVPSRFSGSGSGTDFTLTISSLQPEDVATYYCVQYAQFPLTFGC |
| | | | GTKVEIKSGGGGSEVQLVESGGGLVQPGGSLKLSCAASGFTFNKYAMNWVRQAPGKGM |
| | | | EWVARIRSKYNNYATYYADAVKDRFTISRDDSKNTLYLQMNNLKTEDTAVYYCVRAGN |
| | | | FGSSYISYFAYWGQGTLVTVSSGGGGSGGGGSGGGGSQTVVTQEPSLTVSPGGTVTIT |
| | | | CGSSTGAVTSGNYPNWIQKKPGQAPRGLIGGTKFLAPGTPARFSGSLEGGKAALTLSG |
| | | | VQPEDEAEYYCVLYYSNRWVFGSGTKLTVLGGGGDKTHTCPPCPAPELLGGPSVFLFP |
| | | | PKPKDTLMISRTPEVTCVVVDVSHEDPEVKFNWYVDGVEVHNAKTKPCEEQYGSTYRC |
| | | | VSVLTVLHQDWLNGKEYKCKVSNKALPAPIEKTISKAKGQPREPQVYTLPPSREEMTK |
| | | | NQVSLTCLVKGFYPSDIAVEWESNGQPENNYKTTPPVLDSDGSFFLYSKLTVDKSRWQ |
| | | | QGNVFSCSVMHEALHNHYTQKSLSLSPGKGGGGSGGGGSGGGGGGGGSGGGGSGGGG |
| | | | SDKTHTCPPCPAPELLGGPSVFLFPPKPKDTLMISRTPEVTCVVVDVSHEDPEVKENW |
| | | | YVDGVEVHNAKTKPCEEQYGSTYRCVSVLTVLHQDWLNGKEYKCKVSNKALPAPIEKT |
| | | | ISKAKGQPREPQVYTLPPSREEMTKNQVSLTCLVKGFYPSDIAVEWESNGQPENNYKT |
| | | | TPPVLDSDGSFFLYSKLTVDKSRWQQGNVFSCSVMHEALHNHYTQKSLSLSPGK |
| 2243 | CH3 15-E11 | artificial | QVQLVQSGAEVKKPGASVKVSCKASGYTFTNYWMNWVRQAPGQCLEWMGNIAYGVKGT |
| | CC x I2M x | | NYNQKFQGRVTMTVDTSSSTAYMELSRLRSDDTAVYYCATRYFYVMDYWGQGTLVTVS |
| | scFc-Full | | SGGGGSGGGGSGGGGSDIQMTQSPSSLSASVGDRVTITCRASQDISNYLNWYQQKPGK |
| | Sequence | | VPKLLIYYTSRLHSGVPSRFSGSGSGTDFTLTISSLQPEDVATYYCVQYAQFPLTFGC |
| | | | GTKVEIKSGGGGSEVQLVESGGGLVQPGGSLKLSCAASGFTFNKYAMNWVRQAPGKGM |
| | | | EWVARIRSKYNNYATYYADAVKDRFTISRDDSKNTLYLQMNNLKTEDTAVYYCVRAGN |
| | | | FGTSYISYWAYWGQGTLVTVSSGGGGSGGGGSGGGGSQTVVTQEPSLTVSPGGTVTIT |
| | | | CGSSTGAVTSGNYPNWVQKKPGQAPRGLIGGTKFLAPGTPARFSGSLSGGKAALTLSG |
| | | | VQPEDEAEYYCVLWYSNRWVFGSGTKLTVLGGGGDKTHTCPPCPAPELLGGPSVFLFP |
| | | | PKPKDTLMISRTPEVTCVVVDVSHEDPEVKENWYVDGVEVHNAKTKPCEEQYGSTYRC |
| | | | VSVLTVLHQDWLNGKEYKCKVSNKALPAPIEKTISKAKGQPREPQVYTLPPSREEMTK |
| | | | NQVSLTCLVKGFYPSDIAVEWESNGQPENNYKTTPPVLDSDGSFFLYSKLTVDKSRWQ |
| | | | QGNVFSCSVMHEALHNHYTQKSLSLSPGKGGGGSGGGGSGGGGSGGGGSGGGGSGGGG |
| | | | SDKTHTCPPCPAPELLGGPSVFLFPPKPKDTLMISRTPEVTCVVVDVSHEDPEVKENW |
| | | | YVDGVEVHNAKTKPCEEQYGSTYRCVSVLTVLHQDWLNGKEYKCKVSNKALPAPIEKT |
| | | | ISKAKGQPREPQVYTLPPSREEMTKNQVSLTCLVKGFYPSDIAVEWESNGQPENNYKT |
| | | | TPPVLDSDGSFFLYSKLTVDKSRWQQGNVFSCSVMHEALHNHYTQKSLSLSPGK |
| 2244 | CH3 15-E11 | artificial | QVQLVQSGAEVKKPGASVKVSCKASGYTFTNYWMNWVRQAPGQCLEWMGNIAYGVKGT |
| | CC x I2M2 x | | NYNQKFQGRVTMTVDTSSSTAYMELSRLRSDDTAVYYCATRYFYVMDYWGQGTLVTVS |
| | scFc-Full | | SGGGGSGGGGSGGGGSDIQMTQSPSSLSASVGDRVTITCRASQDISNYLNWYQQKPGK |
| | Sequence | | VPKLLIYYTSRLHSGVPSRFSGSGSGTDFTLTISSLQPEDVATYYCVQYAQFPLTFGC |
| | | | GTKVEIKSGGGGSEVQLVESGGGLVQPGGSLKLSCAASGFTFNKYAINWVREAPGKGL |
| | | | EWVARIRSKYNNYATYYADAVKDRFTISRDDSKNTAYLQMNNLKTEDTAVYYCVRNAN |
| | | | FGTSYISYFAYWGQGTLVTVSSGGGGSGGGGSGGGGSQTVVTQEPSLTVSPGGTVTLT |
| | | | CGSSTGAVTSGNYPNWVQKKPGQAPRGLIGGTKFLAPGTPARFSGSLLGGKAALTLSG |
| | | | VQPEDEAEYYCVLWYSNRWVFGSGTKLTVLGGGGDKTHTCPPCPAPELLGGPSVFLFP |
| | | | PKPKDTLMISRTPEVTCVVVDVSHEDPEVKFNWYVDGVEVHNAKTKPCEEQYGSTYRC |
| | | | VSVLTVLHQDWLNGKEYKCKVSNKALPAPIEKTISKAKGQPREPQVYTLPPSREEMTK |
| | | | NQVSLTCLVKGFYPSDIAVEWESNGQPENNYKTTPPVLDSDGSFFLYSKLTVDKSRWQ |
| | | | QGNVFSCSVMHEALHNHYTQKSLSLSPGKGGGGSGGGGSGGGGSGGGGSGGGGSGGGG |
| | | | SDKTHTCPPCPAPELLGGPSVFLFPPKPKDTLMISRTPEVTCVVVDVSHEDPEVKENW |
| | | | YVDGVEVHNAKTKPCEEQYGSTYRCVSVLTVLHQDWLNGKEYKCKVSNKALPAPIEKT |
| | | | ISKAKGQPREPQVYTLPPSREEMTKNQVSLTCLVKGFYPSDIAVEWESNGQPENNYKT |
| | | | TPPVLDSDGSFFLYSKLTVDKSRWQQGNVFSCSVMHEALHNHYTQKSLSLSPGK |
| 2245 | CH3 G8A 6- | artificial | EVQLLESGGGLVQPGGSLRLSCAASGFSFSSYPINWVRQAPGKGLEWVGVIWTGGGTN |
| | B12 x I2C x | | YASSVKGRFTISRDNSKNTVYLQMNSLRAEDTAVYYCAKSRGVYDFDGRGAMDYWGQG |
| | scFc-Full | | TLVTVSSGGGGSGGGGSGGGGSDIVMTQSPDSLAVSLGERATINCKSSQSLLYSSNQK |
| | Sequence | | NYFAWYQQKPGQPPKLLIYWASTRESGVPDRFSGSGSGTDFTLTISSLQAEDVAVYYC |
| | | | QQYYSYPYTFGQGTKLEIKSGGGGSEVQLVESGGGLVQPGGSLKLSCAASGFTENKYA |
| | | | MNWVRQAPGKGLEWVARIRSKYNNYATYYADSVKDRFTISRDDSKNTAYLQMNNLKTE |
| | | | DTAVYYCVRHGNFGNSYISYWAYWGQGTLVTVSSGGGGSGGGGSGGGGSQTVVTQEPS |
| | | | LTVSPGGTVTLTCGSSTGAVTSGNYPNWVQQKPGQAPRGLIGGTKFLAPGTPARFSGS |
| | | | LLGGKAALTLSGVQPEDEAEYYCVLWYSNRWVFGGGTKLTVLGGGGDKTHTCPPCPAP |
| | | | ELLGGPSVFLFPPKPKDTLMISRTPEVTCVVVDVSHEDPEVKENWYVDGVEVHNAKTK |
| | | | PCEEQYGSTYRCVSVLTVLHQDWLNGKEYKCKVSNKALPAPIEKTISKAKGQPREPQV |
| | | | YTLPPSREEMTKNQVSLTCLVKGFYPSDIAVEWESNGQPENNYKTTPPVLDSDGSFFL |
| | | | YSKLTVDKSRWQQGNVFSCSVMHEALHNHYTQKSLSLSPGKGGGGSGGGGSGGGGSGG |
| | | | GGSGGGGSGGGGSDKTHTCPPCPAPELLGGPSVFLFPPKPKDTLMISRTPEVTCVVVD |
| | | | VSHEDPEVKFNWYVDGVEVHNAKTKPCEEQYGSTYRCVSVLTVLHQDWLNGKEYKCKV |
| | | | SNKALPAPIEKTISKAKGQPREPQVYTLPPSREEMTKNQVSLTCLVKGFYPSDIAVEW |
| | | | ESNGQPENNYKTTPPVLDSDGSFFLYSKLTVDKSRWQQGNVFSCSVMHEALHNHYTQK |
| | | | SLSLSPGK |
| 2246 | CH3 G8A 6- | artificial | EVQLLESGGGLVQPGGSLRLSCAASGFSFSSYPINWVRQAPGKGLEWVGVIWTGGGTN |
| | B12 x I2E x | | YASSVKGRFTISRDNSKNTVYLQMNSLRAEDTAVYYCAKSRGVYDFDGRGAMDYWGQG |
| | scFc-Full | | TLVTVSSGGGGSGGGGSGGGGSDIVMTQSPDSLAVSLGERATINCKSSQSLLYSSNQK |
| | Sequence | | NYFAWYQQKPGQPPKLLIYWASTRESGVPDRFSGSGSGTDFTLTISSLQAEDVAVYYC |
| | | | QQYYSYPYTFGQGTKLEIKSGGGGSEVQLVESGGGLVQPGGSLKLSCAASGFTENKYA |
| | | | INWVRQAPGKGLEWVARIRSKYNNYATYYADAVKDRFTISRDDSKNTVYLQMNNLKTE |
| | | | DTAVYYCARAGNFGSSYISYWAYWGQGTLVTVSSGGGGSGGGGSGGGGSQTVVTQEPS |
| | | | LTVSPGGTVTITCGSSTGAVTSGNYPNWVQKKPGQAPRGLIGGTKFLAPGTPARFSGS |
| | | | LSGGKAALTLSGVQPEDEAEYYCVLWYSNRWVFGSGTKLTVLGGGGDKTHTCPPCPAP |
| | | | ELLGGPSVFLFPPKPKDTLMISRTPEVTCVVVDVSHEDPEVKFNWYVDGVEVHNAKTK |
| | | | PCEEQYGSTYRCVSVLTVLHQDWLNGKEYKCKVSNKALPAPIEKTISKAKGQPREPQV |
| | | | YTLPPSREEMTKNQVSLTCLVKGFYPSDIAVEWESNGQPENNYKTTPPVLDSDGSFFL |
| | | | YSKLTVDKSRWQQGNVFSCSVMHEALHNHYTQKSLSLSPGKGGGGSGGGGSGGGGSGG |
| | | | GGSGGGGSGGGGSDKTHTCPPCPAPELLGGPSVFLFPPKPKDTLMISRTPEVTCVVVD |
| | | | VSHEDPEVKFNWYVDGVEVHNAKTKPCEEQYGSTYRCVSVLTVLHQDWLNGKEYKCKV |
| | | | SNKALPAPIEKTISKAKGQPREPQVYTLPPSREEMTKNQVSLTCLVKGFYPSDIAVEW |
| | | | ESNGQPENNYKTTPPVLDSDGSFFLYSKLTVDKSRWQQGNVFSCSVMHEALHNHYTQK |
| | | | SLSLSPGK |
| 2247 | CH3 G8A 6- | artificial | EVQLLESGGGLVQPGGSLRLSCAASGFSFSSYPINWVRQAPGKGLEWVGVIWTGGGTN |
| | B12 x I2K x | | YASSVKGRFTISRDNSKNTVYLQMNSLRAEDTAVYYCAKSRGVYDFDGRGAMDYWGQG |
| | scFc-Full | | TLVTVSSGGGGSGGGGSGGGGSDIVMTQSPDSLAVSLGERATINCKSSQSLLYSSNQK |
| | Sequence | | NYFAWYQQKPGQPPKLLIYWASTRESGVPDRFSGSGSGTDFTLTISSLQAEDVAVYYC |
| | | | QQYYSYPYTFGQGTKLEIKSGGGGSEVQLVESGGGLVQPGGSLKLSCAASGFTENKYA |
| | | | MNWVRQAPGKGLEWVARIRSKYNNYATYYAEAVKGRFTISRDDSKNTVYLQMNNLKTE |
| | | | DTAVYYCVRNENIGTSYISYWAYWGQGTLVTVSSGGGGSGGGGSGGGGSQTVVTQEPS |
| | | | LTVSPGGTVTITCGSSTGAVTSGNYPNWVQQKPGQAPRGLIGGTKFLAPGTPARFSGS |
| | | | LSGGKAALTLSGVQPEDEAEYYCVLWYSNRWVFGSGTKLTVLGGGGDKTHTCPPCPAP |
| | | | ELLGGPSVFLFPPKPKDTLMISRTPEVTCVVVDVSHEDPEVKENWYVDGVEVHNAKTK |
| | | | PCEEQYGSTYRCVSVLTVLHQDWLNGKEYKCKVSNKALPAPIEKTISKAKGQPREPQV |
| | | | YTLPPSREEMTKNQVSLTCLVKGFYPSDIAVEWESNGQPENNYKTTPPVLDSDGSFFL |
| | | | YSKLTVDKSRWQQGNVFSCSVMHEALHNHYTQKSLSLSPGKGGGGSGGGGSGGGGSGG |
| | | | GGSGGGGSGGGGSDKTHTCPPCPAPELLGGPSVFLFPPKPKDTLMISRTPEVTCVVVD |
| | | | VSHEDPEVKFNWYVDGVEVHNAKTKPCEEQYGSTYRCVSVLTVLHQDWLNGKEYKCKV |
| | | | SNKALPAPIEKTISKAKGQPREPQVYTLPPSREEMTKNQVSLTCLVKGFYPSDIAVEW |
| | | | ESNGQPENNYKTTPPVLDSDGSFFLYSKLTVDKSRWQQGNVFSCSVMHEALHNHYTQK |
| | | | SLSLSPGK |
| 2248 | CH3 G8A 6- | artificial | EVQLLESGGGLVQPGGSLRLSCAASGFSFSSYPINWVRQAPGKGLEWVGVIWTGGGTN |
| | B12 x I2L x | | YASSVKGRFTISRDNSKNTVYLQMNSLRAEDTAVYYCAKSRGVYDFDGRGAMDYWGQG |
| | scFc-Full | | TLVTVSSGGGGSGGGGSGGGGSDIVMTQSPDSLAVSLGERATINCKSSQSLLYSSNQK |
| | Sequence | | NYFAWYQQKPGQPPKLLIYWASTRESGVPDRFSGSGSGTDFTLTISSLQAEDVAVYYC |
| | | | QQYYSYPYTFGQGTKLEIKSGGGGSEVQLVESGGGLVQPGGSLKLSCAASGFTENKYA |
| | | | MNWVRQAPGKGMEWVARIRSKYNNYATYYADAVKDRFTISRDDSKNTLYLQMNNLKTE |
| | | | DTAVYYCVRAGNFGSSYISYFAYWGQGTLVTVSSGGGGSGGGGSGGGGSQTVVTQEPS |
| | | | LTVSPGGTVTITCGSSTGAVTSGNYPNWIQKKPGQAPRGLIGGTKFLAPGTPARFSGS |
| | | | LEGGKAALTLSGVQPEDEAEYYCVLYYSNRWVFGSGTKLTVLGGGGDKTHTCPPCPAP |
| | | | ELLGGPSVFLFPPKPKDTLMISRTPEVTCVVVDVSHEDPEVKENWYVDGVEVHNAKTK |
| | | | PCEEQYGSTYRCVSVLTVLHQDWLNGKEYKCKVSNKALPAPIEKTISKAKGQPREPQV |
| | | | YTLPPSREEMTKNQVSLTCLVKGFYPSDIAVEWESNGQPENNYKTTPPVLDSDGSFFL |
| | | | YSKLTVDKSRWQQGNVFSCSVMHEALHNHYTQKSLSLSPGKGGGGSGGGGSGGGGSGG |
| | | | GGSGGGGSGGGGSDKTHTCPPCPAPELLGGPSVFLFPPKPKDTLMISRTPEVTCVVVD |
| | | | VSHEDPEVKFNWYVDGVEVHNAKTKPCEEQYGSTYRCVSVLTVLHQDWLNGKEYKCKV |
| | | | SNKALPAPIEKTISKAKGQPREPQVYTLPPSREEMTKNQVSLTCLVKGFYPSDIAVEW |
| | | | ESNGQPENNYKTTPPVLDSDGSFFLYSKLTVDKSRWQQGNVFSCSVMHEALHNHYTQK |
| | | | SLSLSPGK |
| 2249 | CH3 G8A 6- | artificial | EVQLLESGGGLVQPGGSLRLSCAASGFSFSSYPINWVRQAPGKGLEWVGVIWTGGGTN |
| | B12 x I2M x | | YASSVKGRFTISRDNSKNTVYLQMNSLRAEDTAVYYCAKSRGVYDFDGRGAMDYWGQG |
| | scFc-Full | | TLVTVSSGGGGSGGGGSGGGGSDIVMTQSPDSLAVSLGERATINCKSSQSLLYSSNQK |
| | Sequence | | NYFAWYQQKPGQPPKLLIYWASTRESGVPDRFSGSGSGTDFTLTISSLQAEDVAVYYC |
| | | | QQYYSYPYTFGQGTKLEIKSGGGGSEVQLVESGGGLVQPGGSLKLSCAASGFTENKYA |
| | | | MNWVRQAPGKGMEWVARIRSKYNNYATYYADAVKDRFTISRDDSKNTLYLQMNNLKTE |
| | | | DTAVYYCVRAGNFGTSYISYWAYWGQGTLVTVSSGGGGSGGGGSGGGGSQTVVTQEPS |
| | | | LTVSPGGTVTITCGSSTGAVTSGNYPNWVQKKPGQAPRGLIGGTKFLAPGTPARFSGS |
| | | | LSGGKAALTLSGVQPEDEAEYYCVLWYSNRWVFGSGTKLTVLGGGGDKTHTCPPCPAP |
| | | | ELLGGPSVFLFPPKPKDTLMISRTPEVTCVVVDVSHEDPEVKENWYVDGVEVHNAKTK |
| | | | PCEEQYGSTYRCVSVLTVLHQDWLNGKEYKCKVSNKALPAPIEKTISKAKGQPREPQV |
| | | | YTLPPSREEMTKNQVSLTCLVKGFYPSDIAVEWESNGQPENNYKTTPPVLDSDGSFFL |
| | | | YSKLTVDKSRWQQGNVFSCSVMHEALHNHYTQKSLSLSPGKGGGGSGGGGSGGGGSGG |
| | | | GGSGGGGSGGGGSDKTHTCPPCPAPELLGGPSVFLFPPKPKDTLMISRTPEVTCVVVD |
| | | | VSHEDPEVKFNWYVDGVEVHNAKTKPCEEQYGSTYRCVSVLTVLHQDWLNGKEYKCKV |
| | | | SNKALPAPIEKTISKAKGQPREPQVYTLPPSREEMTKNQVSLTCLVKGFYPSDIAVEW |
| | | | ESNGQPENNYKTTPPVLDSDGSFFLYSKLTVDKSRWQQGNVFSCSVMHEALHNHYTQK |
| | | | SLSLSPGK |
| 2250 | CH3 G8A 6- | artificial | EVQLLESGGGLVQPGGSLRLSCAASGFSFSSYPINWVRQAPGKGLEWVGVIWTGGGTN |
| | B12 x I2M2 | | YASSVKGRFTISRDNSKNTVYLQMNSLRAEDTAVYYCAKSRGVYDFDGRGAMDYWGQG |
| | x scFc- | | TLVTVSSGGGGSGGGGSGGGGSDIVMTQSPDSLAVSLGERATINCKSSQSLLYSSNQK |
| | Full | | NYFAWYQQKPGQPPKLLIYWASTRESGVPDRFSGSGSGTDFTLTISSLQAEDVAVYYC |
| | Sequence | | QQYYSYPYTFGQGTKLEIKSGGGGSEVQLVESGGGLVQPGGSLKLSCAASGFTENKYA |
| | | | INWVREAPGKGLEWVARIRSKYNNYATYYADAVKDRFTISRDDSKNTAYLQMNNLKTE |
| | | | DTAVYYCVRNANFGTSYISYFAYWGQGTLVTVSSGGGGSGGGGSGGGGSQTVVTQEPS |
| | | | LTVSPGGTVTLTCGSSTGAVTSGNYPNWVQKKPGQAPRGLIGGTKFLAPGTPARFSGS |
| | | | LLGGKAALTLSGVQPEDEAEYYCVLWYSNRWVFGSGTKLTVLGGGGDKTHTCPPCPAP |
| | | | ELLGGPSVFLFPPKPKDTLMISRTPEVTCVVVDVSHEDPEVKENWYVDGVEVHNAKTK |
| | | | PCEEQYGSTYRCVSVLTVLHQDWLNGKEYKCKVSNKALPAPIEKTISKAKGQPREPQV |
| | | | YTLPPSREEMTKNQVSLTCLVKGFYPSDIAVEWESNGQPENNYKTTPPVLDSDGSFFL |
| | | | YSKLTVDKSRWQQGNVFSCSVMHEALHNHYTQKSLSLSPGKGGGGSGGGGSGGGGSGG |
| | | | GGSGGGGSGGGGSDKTHTCPPCPAPELLGGPSVFLFPPKPKDTLMISRTPEVTCVVVD |
| | | | VSHEDPEVKFNWYVDGVEVHNAKTKPCEEQYGSTYRCVSVLTVLHQDWLNGKEYKCKV |
| | | | SNKALPAPIEKTISKAKGQPREPQVYTLPPSREEMTKNQVSLTCLVKGFYPSDIAVEW |
| | | | ESNGQPENNYKTTPPVLDSDGSFFLYSKLTVDKSRWQQGNVFSCSVMHEALHNHYTQK |
| | | | SLSLSPGK |
| 2251 | CH3 R170R | artificial | EVQLLESGGGLVQPGGSLRLSCAASGFTFSSYWMHWVRQTPGKCLEWVSKIDPSDDYT |
| | CC x I2C x | | NYNQKVKGRFTISIDKSKNTLYLQMNSLRAEDTAVYYCARWDYSHFDVWGQGTTVTVS |
| | scFc-Full | | SGGGGSGGGGSGGGGSEIVMTQSPATLSVSPGERATLTCRASSSVSYMHWYQQKPGQA |
| | Sequence | | PRLLIYGTSNLVSGVPARFSGSGSGTEFTLTISSLQSEDFAVYYCQQWSSYPLTFGCG |
| | | | TKVEIKSGGGGSEVQLVESGGGLVQPGGSLKLSCAASGFTFNKYAMNWVRQAPGKGLE |
| | | | WVARIRSKYNNYATYYADSVKDRFTISRDDSKNTAYLQMNNLKTEDTAVYYCVRHGNF |
| | | | GNSYISYWAYWGQGTLVTVSSGGGGSGGGGSGGGGSQTVVTQEPSLTVSPGGTVTLTC |
| | | | GSSTGAVTSGNYPNWVQQKPGQAPRGLIGGTKFLAPGTPARFSGSLLGGKAALTLSGV |
| | | | QPEDEAEYYCVLWYSNRWVFGGGTKLTVLGGGGDKTHTCPPCPAPELLGGPSVFLFPP |
| | | | KPKDTLMISRTPEVTCVVVDVSHEDPEVKENWYVDGVEVHNAKTKPCEEQYGSTYRCV |
| | | | SVLTVLHQDWLNGKEYKCKVSNKALPAPIEKTISKAKGQPREPQVYTLPPSREEMTKN |
| | | | QVSLTCLVKGFYPSDIAVEWESNGQPENNYKTTPPVLDSDGSFFLYSKLTVDKSRWQQ |
| | | | GNVFSCSVMHEALHNHYTQKSLSLSPGKGGGGSGGGGSGGGGSGGGGSGGGGSGGGGS |
| | | | DKTHTCPPCPAPELLGGPSVFLFPPKPKDTLMISRTPEVTCVVVDVSHEDPEVKENWY |
| | | | VDGVEVHNAKTKPCEEQYGSTYRCVSVLTVLHQDWLNGKEYKCKVSNKALPAPIEKTI |
| | | | SKAKGQPREPQVYTLPPSREEMTKNQVSLTCLVKGFYPSDIAVEWESNGQPENNYKTT |
| | | | PPVLDSDGSFFLYSKLTVDKSRWQQGNVFSCSVMHEALHNHYTQKSLSLSPGK |
| 2252 | CH3 R170R | artificial | EVQLLESGGGLVQPGGSLRLSCAASGFTFSSYWMHWVRQTPGKCLEWVSKIDPSDDYT |
| | CC x I2E x | | NYNQKVKGRFTISIDKSKNTLYLQMNSLRAEDTAVYYCARWDYSHFDVWGQGTTVTVS |
| | scFc-Full | | SGGGGSGGGGSGGGGSEIVMTQSPATLSVSPGERATLTCRASSSVSYMHWYQQKPGQA |
| | Sequence | | PRLLIYGTSNLVSGVPARFSGSGSGTEFTLTISSLQSEDFAVYYCQQWSSYPLTFGCG |
| | | | TKVEIKSGGGGSEVQLVESGGGLVQPGGSLKLSCAASGFTFNKYAINWVRQAPGKGLE |
| | | | WVARIRSKYNNYATYYADAVKDRFTISRDDSKNTVYLQMNNLKTEDTAVYYCARAGNF |
| | | | GSSYISYWAYWGQGTLVTVSSGGGGSGGGGSGGGGSQTVVTQEPSLTVSPGGTVTITC |
| | | | GSSTGAVTSGNYPNWVQKKPGQAPRGLIGGTKFLAPGTPARFSGSLSGGKAALTLSGV |
| | | | QPEDEAEYYCVLWYSNRWVFGSGTKLTVLGGGGDKTHTCPPCPAPELLGGPSVFLFPP |
| | | | KPKDTLMISRTPEVTCVVVDVSHEDPEVKENWYVDGVEVHNAKTKPCEEQYGSTYRCV |
| | | | SVLTVLHQDWLNGKEYKCKVSNKALPAPIEKTISKAKGQPREPQVYTLPPSREEMTKN |
| | | | QVSLTCLVKGFYPSDIAVEWESNGQPENNYKTTPPVLDSDGSFFLYSKLTVDKSRWQQ |
| | | | GNVFSCSVMHEALHNHYTQKSLSLSPGKGGGGSGGGGSGGGGSGGGGSGGGGSGGGGS |
| | | | DKTHTCPPCPAPELLGGPSVFLFPPKPKDTLMISRTPEVTCVVVDVSHEDPEVKENWY |
| | | | VDGVEVHNAKTKPCEEQYGSTYRCVSVLTVLHQDWLNGKEYKCKVSNKALPAPIEKTI |
| | | | SKAKGQPREPQVYTLPPSREEMTKNQVSLTCLVKGFYPSDIAVEWESNGQPENNYKTT |
| | | | PPVLDSDGSFFLYSKLTVDKSRWQQGNVFSCSVMHEALHNHYTQKSLSLSPGK |
| 2253 | CH3 R170R | artificial | EVQLLESGGGLVQPGGSLRLSCAASGFTFSSYWMHWVRQTPGKCLEWVSKIDPSDDYT |
| | CC x I2K x | | NYNQKVKGRFTISIDKSKNTLYLQMNSLRAEDTAVYYCARWDYSHFDVWGQGTTVTVS |
| | scFc-Full | | SGGGGSGGGGSGGGGSEIVMTQSPATLSVSPGERATLTCRASSSVSYMHWYQQKPGQA |
| | Sequence | | PRLLIYGTSNLVSGVPARFSGSGSGTEFTLTISSLQSEDFAVYYCQQWSSYPLTFGCG |
| | | | TKVEIKSGGGGSEVQLVESGGGLVQPGGSLKLSCAASGFTFNKYAMNWVRQAPGKGLE |
| | | | WVARIRSKYNNYATYYAEAVKGRFTISRDDSKNTVYLQMNNLKTEDTAVYYCVRNENI |
| | | | GTSYISYWAYWGQGTLVTVSSGGGGSGGGGSGGGGSQTVVTQEPSLTVSPGGTVTITC |
| | | | GSSTGAVTSGNYPNWVQQKPGQAPRGLIGGTKFLAPGTPARFSGSLSGGKAALTLSGV |
| | | | QPEDEAEYYCVLWYSNRWVFGSGTKLTVLGGGGDKTHTCPPCPAPELLGGPSVFLFPP |
| | | | KPKDTLMISRTPEVTCVVVDVSHEDPEVKFNWYVDGVEVHNAKTKPCEEQYGSTYRCV |
| | | | SVLTVLHQDWLNGKEYKCKVSNKALPAPIEKTISKAKGQPREPQVYTLPPSREEMTKN |
| | | | QVSLTCLVKGFYPSDIAVEWESNGQPENNYKTTPPVLDSDGSFFLYSKLTVDKSRWQQ |
| | | | GNVFSCSVMHEALHNHYTQKSLSLSPGKGGGGSGGGGSGGGGSGGGGSGGGGSGGGGS |
| | | | DKTHTCPPCPAPELLGGPSVFLFPPKPKDTLMISRTPEVTCVVVDVSHEDPEVKENWY |
| | | | VDGVEVHNAKTKPCEEQYGSTYRCVSVLTVLHQDWLNGKEYKCKVSNKALPAPIEKTI |
| | | | SKAKGQPREPQVYTLPPSREEMTKNQVSLTCLVKGFYPSDIAVEWESNGQPENNYKTT |
| | | | PPVLDSDGSFFLYSKLTVDKSRWQQGNVFSCSVMHEALHNHYTQKSLSLSPGK |
| 2254 | CH3 R170R | artificial | EVQLLESGGGLVQPGGSLRLSCAASGFTFSSYWMHWVRQTPGKCLEWVSKIDPSDDYT |
| | CC x I2L x | | NYNQKVKGRFTISIDKSKNTLYLQMNSLRAEDTAVYYCARWDYSHFDVWGQGTTVTVS |
| | scFc-Full | | SGGGGSGGGGSGGGGSEIVMTQSPATLSVSPGERATLTCRASSSVSYMHWYQQKPGQA |
| | Sequence | | PRLLIYGTSNLVSGVPARFSGSGSGTEFTLTISSLQSEDFAVYYCQQWSSYPLTFGCG |
| | | | TKVEIKSGGGGSEVQLVESGGGLVQPGGSLKLSCAASGFTFNKYAMNWVRQAPGKGME |
| | | | WVARIRSKYNNYATYYADAVKDRFTISRDDSKNTLYLQMNNLKTEDTAVYYCVRAGNF |
| | | | GSSYISYFAYWGQGTLVTVSSGGGGSGGGGSGGGGSQTVVTQEPSLTVSPGGTVTITC |
| | | | GSSTGAVTSGNYPNWIQKKPGQAPRGLIGGTKFLAPGTPARFSGSLEGGKAALTLSGV |
| | | | QPEDEAEYYCVLYYSNRWVFGSGTKLTVLGGGGDKTHTCPPCPAPELLGGPSVFLFPP |
| | | | KPKDTLMISRTPEVTCVVVDVSHEDPEVKFNWYVDGVEVHNAKTKPCEEQYGSTYRCV |
| | | | SVLTVLHQDWLNGKEYKCKVSNKALPAPIEKTISKAKGQPREPQVYTLPPSREEMTKN |
| | | | QVSLTCLVKGFYPSDIAVEWESNGQPENNYKTTPPVLDSDGSFFLYSKLTVDKSRWQQ |
| | | | GNVFSCSVMHEALHNHYTQKSLSLSPGKGGGGSGGGGSGGGGSGGGGSGGGGSGGGGS |
| | | | DKTHTCPPCPAPELLGGPSVFLFPPKPKDTLMISRTPEVTCVVVDVSHEDPEVKENWY |
| | | | VDGVEVHNAKTKPCEEQYGSTYRCVSVLTVLHQDWLNGKEYKCKVSNKALPAPIEKTI |
| | | | SKAKGQPREPQVYTLPPSREEMTKNQVSLTCLVKGFYPSDIAVEWESNGQPENNYKTT |
| | | | PPVLDSDGSFFLYSKLTVDKSRWQQGNVFSCSVMHEALHNHYTQKSLSLSPGK |
| 2255 | CH3 R170R | artificial | EVQLLESGGGLVQPGGSLRLSCAASGFTFSSYWMHWVRQTPGKCLEWVSKIDPSDDYT |
| | CC x I2M x | | NYNQKVKGRFTISIDKSKNTLYLQMNSLRAEDTAVYYCARWDYSHEDVWGQGTTVTVS |
| | scFc-Full | | SGGGGSGGGGSGGGGSEIVMTQSPATLSVSPGERATLTCRASSSVSYMHWYQQKPGQA |
| | Sequence | | PRLLIYGTSNLVSGVPARFSGSGSGTEFTLTISSLQSEDFAVYYCQQWSSYPLTFGCG |
| | | | TKVEIKSGGGGSEVQLVESGGGLVQPGGSLKLSCAASGFTENKYAMNWVRQAPGKGME |
| | | | WVARIRSKYNNYATYYADAVKDRFTISRDDSKNTLYLQMNNLKTEDTAVYYCVRAGNE |
| | | | GTSYISYWAYWGQGTLVTVSSGGGGSGGGGSGGGGSQTVVTQEPSLTVSPGGTVTITC |
| | | | GSSTGAVTSGNYPNWVQKKPGQAPRGLIGGTKFLAPGTPARFSGSLSGGKAALTLSGV |
| | | | QPEDEAEYYCVLWYSNRWVFGSGTKLTVLGGGGDKTHTCPPCPAPELLGGPSVFLFPP |
| | | | KPKDTLMISRTPEVTCVVVDVSHEDPEVKFNWYVDGVEVHNAKTKPCEEQYGSTYRCV |
| | | | SVLTVLHQDWLNGKEYKCKVSNKALPAPIEKTISKAKGQPREPQVYTLPPSREEMTKN |
| | | | QVSLTCLVKGFYPSDIAVEWESNGQPENNYKTTPPVLDSDGSFFLYSKLTVDKSRWQQ |
| | | | GNVFSCSVMHEALHNHYTQKSLSLSPGKGGGGSGGGGSGGGGSGGGGSGGGGSGGGGS |
| | | | DKTHTCPPCPAPELLGGPSVFLFPPKPKDTLMISRTPEVTCVVVDVSHEDPEVKENWY |
| | | | VDGVEVHNAKTKPCEEQYGSTYRCVSVLTVLHQDWLNGKEYKCKVSNKALPAPIEKTI |
| | | | SKAKGQPREPQVYTLPPSREEMTKNQVSLTCLVKGFYPSDIAVEWESNGQPENNYKTT |
| | | | PPVLDSDGSFFLYSKLTVDKSRWQQGNVFSCSVMHEALHNHYTQKSLSLSPGK |
| 2256 | CH3 R170R | artificial | EVQLLESGGGLVQPGGSLRLSCAASGFTFSSYWMHWVRQTPGKCLEWVSKIDPSDDYT |
| | CC x I2M2 x | | NYNQKVKGRFTISIDKSKNTLYLQMNSLRAEDTAVYYCARWDYSHFDVWGQGTTVTVS |
| | scFc-Full | | SGGGGSGGGGSGGGGSEIVMTQSPATLSVSPGERATLTCRASSSVSYMHWYQQKPGQA |
| | Sequence | | PRLLIYGTSNLVSGVPARFSGSGSGTEFTLTISSLQSEDFAVYYCQQWSSYPLTFGCG |
| | | | TKVEIKSGGGGSEVQLVESGGGLVQPGGSLKLSCAASGFTFNKYAINWVREAPGKGLE |
| | | | WVARIRSKYNNYATYYADAVKDRFTISRDDSKNTAYLQMNNLKTEDTAVYYCVRNANF |
| | | | GTSYISYFAYWGQGTLVTVSSGGGGSGGGGSGGGGSQTVVTQEPSLTVSPGGTVTLTC |
| | | | GSSTGAVTSGNYPNWVQKKPGQAPRGLIGGTKFLAPGTPARFSGSLLGGKAALTLSGV |
| | | | QPEDEAEYYCVLWYSNRWVFGSGTKLTVLGGGGDKTHTCPPCPAPELLGGPSVFLFPP |
| | | | KPKDTLMISRTPEVTCVVVDVSHEDPEVKFNWYVDGVEVHNAKTKPCEEQYGSTYRCV |
| | | | SVLTVLHQDWLNGKEYKCKVSNKALPAPIEKTISKAKGQPREPQVYTLPPSREEMTKN |
| | | | QVSLTCLVKGFYPSDIAVEWESNGQPENNYKTTPPVLDSDGSFFLYSKLTVDKSRWQQ |
| | | | GNVFSCSVMHEALHNHYTQKSLSLSPGKGGGGSGGGGSGGGGSGGGGSGGGGSGGGGS |
| | | | DKTHTCPPCPAPELLGGPSVFLFPPKPKDTLMISRTPEVTCVVVDVSHEDPEVKENWY |
| | | | VDGVEVHNAKTKPCEEQYGSTYRCVSVLTVLHQDWLNGKEYKCKVSNKALPAPIEKTI |
| | | | SKAKGQPREPQVYTLPPSREEMTKNQVSLTCLVKGFYPSDIAVEWESNGQPENNYKTT |
| | | | PPVLDSDGSFFLYSKLTVDKSRWQQGNVFSCSVMHEALHNHYTQKSLSLSPGK |
| 2257 | CL1 7-D7 CC | artificial | NYYMH |
| | -HCDR1 | | |
| 2258 | CL1 7-D7 CC | artificial | WINPTSGGANYAQKFQG |
| | -HCDR2 | | |
| 2259 | CL1 7-D7 CC | artificial | ESHAIQEGIWFDY |
| | -HCDR3 | | |
| 2260 | CL1 7-D7 CC | artificial | RASQSISNYLN |
| | -LCDR1 | | |
| 2261 | CL1 7-D7 CC | artificial | DASSLQS |
| | -LCDR2 | | |
| 2262 | CL1 7-D7 CC | artificial | QQSYSFPLT |
| | -LCDR3 | | |
| 2263 | CL1 7-D7 CC | artificial | QVQLVQSGAEVKKPGASVKVSCKASGYTFTNYYMHWVRQAPGQCLEWMGWINPTSGGA |
| | -VH | | NYAQKFQGRVTMTRDTSISTAYMELSRLRSDDTAVYFCARESHAIQEGIWFDYWGQGT |
| | | | LVTVSS |
| 2264 | CL1 7-D7 CC | artificial | DIQMTQSPSSLSASVGDRVTISCRASQSISNYLNWYQQKPGKAPKLLIYDASSLQSGV |
| | -VL | | PSRFSGSGSGTDFTLTISSLQPEDFATYYCQQSYSFPLTFGCGTKVEIK |
| 2265 | CL1 7-D7 CC | artificial | EIQMTQSPSSLSASVGDRVTISCRASQSISNYLNWYQQKPGKAPKLLIYDASSLQSGV |
| | EI GQ-VL | | PSRFSGSGSGTDFTLTISSLQPEDFATYYCQQSYSFPLTFGCGTKVEIK |
| 2266 | CL1 7-D7 CC | artificial | QVQLVQSGAEVKKPGASVKVSCKASGYTFTNYYMHWVRQAPGQCLEWMGWINPTSGGA |
| | x I2Cx scFc | | NYAQKFQGRVTMTRDTSISTAYMELSRLRSDDTAVYFCARESHAIQEGIWFDYWGQGT |
| | -Full | | LVTVSSGGGGSGGGGSGGGGSDIQMTQSPSSLSASVGDRVTISCRASQSISNYLNWYQ |
| | Sequence | | QKPGKAPKLLIYDASSLQSGVPSRFSGSGSGTDFTLTISSLQPEDFATYYCQQSYSFP |
| | | | LTFGCGTKVEIKSGGGGSEVQLVESGGGLVQPGGSLKLSCAASGFTENKYAMNWVRQA |
| | | | PGKGLEWVARIRSKYNNYATYYADSVKDRFTISRDDSKNTAYLQMNNLKTEDTAVYYC |
| | | | VRHGNFGNSYISYWAYWGQGTLVTVSSGGGGSGGGGSGGGGSQTVVTQEPSLTVSPGG |
| | | | TVTLTCGSSTGAVTSGNYPNWVQQKPGQAPRGLIGGTKFLAPGTPARFSGSLLGGKAA |
| | | | LTLSGVQPEDEAEYYCVLWYSNRWVFGGGTKLTVLGGGGDKTHTCPPCPAPELLGGPS |
| | | | VFLFPPKPKDTLMISRTPEVTCVVVDVSHEDPEVKFNWYVDGVEVHNAKTKPCEEQYG |
| | | | STYRCVSVLTVLHQDWLNGKEYKCKVSNKALPAPIEKTISKAKGQPREPQVYTLPPSR |
| | | | EEMTKNQVSLTCLVKGFYPSDIAVEWESNGQPENNYKTTPPVLDSDGSFFLYSKLTVD |
| | | | KSRWQQGNVFSCSVMHEALHNHYTQKSLSLSPGKGGGGSGGGGSGGGGSGGGGSGGGG |
| | | | SGGGGSDKTHTCPPCPAPELLGGPSVFLFPPKPKDTLMISRTPEVTCVVVDVSHEDPE |
| | | | VKFNWYVDGVEVHNAKTKPCEEQYGSTYRCVSVLTVLHQDWLNGKEYKCKVSNKALPA |
| | | | PIEKTISKAKGQPREPQVYTLPPSREEMTKNQVSLTCLVKGFYPSDIAVEWESNGQPE |
| | | | NNYKTTPPVLDSDGSFFLYSKLTVDKSRWQQGNVFSCSVMHEALHNHYTQKSLSLSPG |
| | | | K |
| 2267 | CL1 7-D7 CC | artificial | QVQLVQSGAEVKKPGASVKVSCKASGYTFTNYYMHWVRQAPGQCLEWMGWINPTSGGA |
| | x I2Ex scFc | | NYAQKFQGRVTMTRDTSISTAYMELSRLRSDDTAVYFCARESHAIQEGIWFDYWGQGT |
| | -Full | | LVTVSSGGGGSGGGGSGGGGSDIQMTQSPSSLSASVGDRVTISCRASQSISNYLNWYQ |
| | Sequence | | QKPGKAPKLLIYDASSLQSGVPSRFSGSGSGTDFTLTISSLQPEDFATYYCQQSYSFP |
| | | | LTFGCGTKVEIKSGGGGSEVQLVESGGGLVQPGGSLKLSCAASGFTFNKYAINWVRQA |
| | | | PGKGLEWVARIRSKYNNYATYYADAVKDRFTISRDDSKNTVYLQMNNLKTEDTAVYYC |
| | | | ARAGNFGSSYISYWAYWGQGTLVTVSSGGGGSGGGGSGGGGSQTVVTQEPSLTVSPGG |
| | | | TVTITCGSSTGAVTSGNYPNWVQKKPGQAPRGLIGGTKFLAPGTPARFSGSLSGGKAA |
| | | | LTLSGVQPEDEAEYYCVLWYSNRWVFGSGTKLTVLGGGGDKTHTCPPCPAPELLGGPS |
| | | | VFLFPPKPKDTLMISRTPEVTCVVVDVSHEDPEVKFNWYVDGVEVHNAKTKPCEEQYG |
| | | | STYRCVSVLTVLHQDWLNGKEYKCKVSNKALPAPIEKTISKAKGQPREPQVYTLPPSR |
| | | | EEMTKNQVSLTCLVKGFYPSDIAVEWESNGQPENNYKTTPPVLDSDGSFFLYSKLTVD |
| | | | KSRWQQGNVFSCSVMHEALHNHYTQKSLSLSPGKGGGGSGGGGSGGGGSGGGGSGGGG |
| | | | SGGGGSDKTHTCPPCPAPELLGGPSVFLFPPKPKDTLMISRTPEVTCVVVDVSHEDPE |
| | | | VKFNWYVDGVEVHNAKTKPCEEQYGSTYRCVSVLTVLHQDWLNGKEYKCKVSNKALPA |
| | | | PIEKTISKAKGQPREPQVYTLPPSREEMTKNQVSLTCLVKGFYPSDIAVEWESNGQPE |
| | | | NNYKTTPPVLDSDGSFFLYSKLTVDKSRWQQGNVFSCSVMHEALHNHYTQKSLSLSPG |
| | | | K |
| 2268 | CL1 7-D7 CC | artificial | QVQLVQSGAEVKKPGASVKVSCKASGYTFTNYYMHWVRQAPGQCLEWMGWINPTSGGA |
| | x 12Kx scFc | | NYAQKFQGRVTMTRDTSISTAYMELSRLRSDDTAVYFCARESHAIQEGIWFDYWGQGT |
| | -Full | | LVTVSSGGGGSGGGGSGGGGSDIQMTQSPSSLSASVGDRVTISCRASQSISNYLNWYQ |
| | Sequence | | QKPGKAPKLLIYDASSLQSGVPSRFSGSGSGTDFTLTISSLQPEDFATYYCQQSYSFP |
| | | | LTFGCGTKVEIKSGGGGSEVQLVESGGGLVQPGGSLKLSCAASGFTFNKYAMNWVRQA |
| | | | PGKGLEWVARIRSKYNNYATYYAEAVKGRFTISRDDSKNTVYLQMNNLKTEDTAVYYC |
| | | | VRNENIGTSYISYWAYWGQGTLVTVSSGGGGSGGGGSGGGGSQTVVTQEPSLTVSPGG |
| | | | TVTITCGSSTGAVTSGNYPNWVQQKPGQAPRGLIGGTKFLAPGTPARFSGSLSGGKAA |
| | | | LTLSGVQPEDEAEYYCVLWYSNRWVFGSGTKLTVLGGGGDKTHTCPPCPAPELLGGPS |
| | | | VFLFPPKPKDTLMISRTPEVTCVVVDVSHEDPEVKFNWYVDGVEVHNAKTKPCEEQYG |
| | | | STYRCVSVLTVLHQDWLNGKEYKCKVSNKALPAPIEKTISKAKGQPREPQVYTLPPSR |
| | | | EEMTKNQVSLTCLVKGFYPSDIAVEWESNGQPENNYKTTPPVLDSDGSFFLYSKLTVD |
| | | | KSRWQQGNVFSCSVMHEALHNHYTQKSLSLSPGKGGGGSGGGGSGGGGSGGGGSGGGG |
| | | | SGGGGSDKTHTCPPCPAPELLGGPSVFLFPPKPKDTLMISRTPEVTCVVVDVSHEDPE |
| | | | VKFNWYVDGVEVHNAKTKPCEEQYGSTYRCVSVLTVLHQDWLNGKEYKCKVSNKALPA |
| | | | PIEKTISKAKGQPREPQVYTLPPSREEMTKNQVSLTCLVKGFYPSDIAVEWESNGQPE |
| | | | NNYKTTPPVLDSDGSFFLYSKLTVDKSRWQQGNVFSCSVMHEALHNHYTQKSLSLSPG |
| | | | K |
| 2269 | CL1 7-D7 CC | artificial | QVQLVQSGAEVKKPGASVKVSCKASGYTFTNYYMHWVRQAPGQCLEWMGWINPTSGGA |
| | x I2Lx scFc | | NYAQKFQGRVTMTRDTSISTAYMELSRLRSDDTAVYFCARESHAIQEGIWFDYWGQGT |
| | -Full | | LVTVSSGGGGSGGGGSGGGGSDIQMTQSPSSLSASVGDRVTISCRASQSISNYLNWYQ |
| | Sequence | | QKPGKAPKLLIYDASSLQSGVPSRFSGSGSGTDFTLTISSLQPEDFATYYCQQSYSFP |
| | | | LTFGCGTKVEIKSGGGGSEVQLVESGGGLVQPGGSLKLSCAASGFTFNKYAMNWVRQA |
| | | | PGKGMEWVARIRSKYNNYATYYADAVKDRFTISRDDSKNTLYLQMNNLKTEDTAVYYC |
| | | | VRAGNFGSSYISYFAYWGQGTLVTVSSGGGGSGGGGSGGGGSQTVVTQEPSLTVSPGG |
| | | | TVTITCGSSTGAVTSGNYPNWIQKKPGQAPRGLIGGTKFLAPGTPARFSGSLEGGKAA |
| | | | LTLSGVQPEDEAEYYCVLYYSNRWVFGSGTKLTVLGGGGDKTHTCPPCPAPELLGGPS |
| | | | VFLFPPKPKDTLMISRTPEVTCVVVDVSHEDPEVKENWYVDGVEVHNAKTKPCEEQYG |
| | | | STYRCVSVLTVLHQDWLNGKEYKCKVSNKALPAPIEKTISKAKGQPREPQVYTLPPSR |
| | | | EEMTKNQVSLTCLVKGFYPSDIAVEWESNGQPENNYKTTPPVLDSDGSFFLYSKLTVD |
| | | | KSRWQQGNVFSCSVMHEALHNHYTQKSLSLSPGKGGGGSGGGGSGGGGSGGGGSGGGG |
| | | | SGGGGSDKTHTCPPCPAPELLGGPSVFLFPPKPKDTLMISRTPEVTCVVVDVSHEDPE |
| | | | VKFNWYVDGVEVHNAKTKPCEEQYGSTYRCVSVLTVLHQDWLNGKEYKCKVSNKALPA |
| | | | PIEKTISKAKGQPREPQVYTLPPSREEMTKNQVSLTCLVKGFYPSDIAVEWESNGQPE |
| | | | NNYKTTPPVLDSDGSFFLYSKLTVDKSRWQQGNVFSCSVMHEALHNHYTQKSLSLSPG |
| | | | K |
| 2270 | CL1 7-D7 CC | artificial | QVQLVQSGAEVKKPGASVKVSCKASGYTFTNYYMHWVRQAPGQCLEWMGWINPTSGGA |
| | x I2M2x | | NYAQKFQGRVTMTRDTSISTAYMELSRLRSDDTAVYFCARESHAIQEGIWFDYWGQGT |
| | ScFc-Full | | LVTVSSGGGGSGGGGSGGGGSDIQMTQSPSSLSASVGDRVTISCRASQSISNYLNWYQ |
| | Sequence | | QKPGKAPKLLIYDASSLQSGVPSRFSGSGSGTDFTLTISSLQPEDFATYYCQQSYSFP |
| | | | LTFGCGTKVEIKSGGGGSEVQLVESGGGLVQPGGSLKLSCAASGFTENKYAINWVREA |
| | | | PGKGLEWVARIRSKYNNYATYYADAVKDRFTISRDDSKNTAYLQMNNLKTEDTAVYYC |
| | | | VRNANFGTSYISYFAYWGQGTLVTVSSGGGGSGGGGSGGGGSQTVVTQEPSLTVSPGG |
| | | | TVTLTCGSSTGAVTSGNYPNWVQKKPGQAPRGLIGGTKFLAPGTPARFSGSLLGGKAA |
| | | | LTLSGVQPEDEAEYYCVLWYSNRWVFGSGTKLTVLGGGGDKTHTCPPCPAPELLGGPS |
| | | | VFLFPPKPKDTLMISRTPEVTCVVVDVSHEDPEVKENWYVDGVEVHNAKTKPCEEQYG |
| | | | STYRCVSVLTVLHQDWLNGKEYKCKVSNKALPAPIEKTISKAKGQPREPQVYTLPPSR |
| | | | EEMTKNQVSLTCLVKGFYPSDIAVEWESNGQPENNYKTTPPVLDSDGSFFLYSKLTVD |
| | | | KSRWQQGNVFSCSVMHEALHNHYTQKSLSLSPGKGGGGSGGGGSGGGGSGGGGSGGGG |
| | | | SGGGGSDKTHTCPPCPAPELLGGPSVFLFPPKPKDTLMISRTPEVTCVVVDVSHEDPE |
| | | | VKFNWYVDGVEVHNAKTKPCEEQYGSTYRCVSVLTVLHQDWLNGKEYKCKVSNKALPA |
| | | | PIEKTISKAKGQPREPQVYTLPPSREEMTKNQVSLTCLVKGFYPSDIAVEWESNGQPE |
| | | | NNYKTTPPVLDSDGSFFLYSKLTVDKSRWQQGNVFSCSVMHEALHNHYTQKSLSLSPG |
| | | | K |
| 2271 | CL1 7-D7 CC | artificial | QVQLVQSGAEVKKPGASVKVSCKASGYTFTNYYMHWVRQAPGQCLEWMGWINPTSGGA |
| | x I2Mx scFc | | NYAQKFQGRVTMTRDTSISTAYMELSRLRSDDTAVYFCARESHAIQEGIWFDYWGQGT |
| | -Full | | LVTVSSGGGGSGGGGSGGGGSDIQMTQSPSSLSASVGDRVTISCRASQSISNYLNWYQ |
| | Sequence | | QKPGKAPKLLIYDASSLQSGVPSRFSGSGSGTDFTLTISSLQPEDFATYYCQQSYSFP |
| | | | LTFGCGTKVEIKSGGGGSEVQLVESGGGLVQPGGSLKLSCAASGFTFNKYAMNWVRQA |
| | | | PGKGMEWVARIRSKYNNYATYYADAVKDRFTISRDDSKNTLYLQMNNLKTEDTAVYYC |
| | | | VRAGNFGTSYISYWAYWGQGTLVTVSSGGGGSGGGGSGGGGSQTVVTQEPSLTVSPGG |
| | | | TVTITCGSSTGAVTSGNYPNWVQKKPGQAPRGLIGGTKFLAPGTPARFSGSLSGGKAA |
| | | | LTLSGVQPEDEAEYYCVLWYSNRWVFGSGTKLTVLGGGGDKTHTCPPCPAPELLGGPS |
| | | | VELFPPKPKDTLMISRTPEVTCVVVDVSHEDPEVKFNWYVDGVEVHNAKTKPCEEQYG |
| | | | STYRCVSVLTVLHQDWLNGKEYKCKVSNKALPAPIEKTISKAKGQPREPQVYTLPPSR |
| | | | EEMTKNQVSLTCLVKGFYPSDIAVEWESNGQPENNYKTTPPVLDSDGSFFLYSKLTVD |
| | | | KSRWQQGNVFSCSVMHEALHNHYTQKSLSLSPGKGGGGSGGGGSGGGGSGGGGSGGGG |
| | | | SGGGGSDKTHTCPPCPAPELLGGPSVFLFPPKPKDTLMISRTPEVTCVVVDVSHEDPE |
| | | | VKFNWYVDGVEVHNAKTKPCEEQYGSTYRCVSVLTVLHQDWLNGKEYKCKVSNKALPA |
| | | | PIEKTISKAKGQPREPQVYTLPPSREEMTKNQVSLTCLVKGFYPSDIAVEWESNGQPE |
| | | | NNYKTTPPVLDSDGSFFLYSKLTVDKSRWQQGNVFSCSVMHEALHNHYTQKSLSLSPG |
| | | | K |
| 2272 | CS | artificial | SSWMN |
| | PDL241.12.LH | | |
| | CC- | | |
| | HCDR1 | | |
| 2273 | CS | artificial | RIYPGDADAKYNAKFKG |
| | PDL241.12.LH | | |
| | CC- | | |
| | HCDR2 | | |
| 2274 | CS | artificial | STMIATGAMDY |
| | PDL241.12.LH | | |
| | CC- | | |
| | HCDR3 | | |
| 2275 | CS | artificial | KASQDVSTAVA |
| | PDL241.12.LH | | |
| | CC- | | |
| | LCDR1 | | |
| 2276 | CS | artificial | SASYRYT |
| | PDL241.12.LH | | |
| | CC- | | |
| | LCDR2 | | |
| 2277 | CS | artificial | QQHYSTPPYT |
| | PDL241.12.LH | | |
| | CC- | | |
| | LCDR3 | | |
| 2278 | CS | artificial | QVQLVQSGAEVKKPGASVKVSCKASGYAFSSSWMNWVRQAPGQCLEWIGRIYPGDADA |
| | PDL241.12.LH | | KYNAKFKGKATLTADKSTSTAYMELSSLRSEDTAVYYCARSTMIATGAMDYWGQGTLV |
| | CC-VH | | TVSS |
| 2279 | CS | artificial | DIQMTQSPSSLSASVGDRVTITCKASQDVSTAVAWYQQKPGKAPKLLIYSASYRYTGV |
| | PDL241.12.LH | | PDRFTGSGSGTDFTLTISSLQPEDFATYYCQQHYSTPPYTFGCGTKVEIKR |
| | CC-VL | | |
| 2280 | CS | artificial | DIQMTQSPSSLSASVGDRVTITCKASQDVSTAVAWYQQKPGKAPKLLIYSASYRYTGV |
| | PDL241.12_LH | | PDRFTGSGSGTDFTLTISSLQPEDFATYYCQQHYSTPPYTFGCGTKVEIKRGGGGSGG |
| | CC x I2C | | GGSGGGGSQVQLVQSGAEVKKPGASVKVSCKASGYAFSSSWMNWVRQAPGQCLEWIGR |
| | x scFc- | | IYPGDADAKYNAKFKGKATLTADKSTSTAYMELSSLRSEDTAVYYCARSTMIATGAMD |
| | Full | | YWGQGTLVTVSSSGGGGSEVQLVESGGGLVQPGGSLKLSCAASGFTENKYAMNWVRQA |
| | Sequence | | PGKGLEWVARIRSKYNNYATYYADSVKDRFTISRDDSKNTAYLQMNNLKTEDTAVYYC |
| | | | VRHGNFGNSYISYWAYWGQGTLVTVSSGGGGSGGGGSGGGGSQTVVTQEPSLTVSPGG |
| | | | TVTLTCGSSTGAVTSGNYPNWVQQKPGQAPRGLIGGTKFLAPGTPARFSGSLLGGKAA |
| | | | LTLSGVQPEDEAEYYCVLWYSNRWVFGGGTKLTVLGGGGDKTHTCPPCPAPELLGGPS |
| | | | VFLFPPKPKDTLMISRTPEVTCVVVDVSHEDPEVKENWYVDGVEVHNAKTKPCEEQYG |
| | | | STYRCVSVLTVLHQDWLNGKEYKCKVSNKALPAPIEKTISKAKGQPREPQVYTLPPSR |
| | | | EEMTKNQVSLTCLVKGFYPSDIAVEWESNGQPENNYKTTPPVLDSDGSFFLYSKLTVD |
| | | | KSRWQQGNVFSCSVMHEALHNHYTQKSLSLSPGKGGGGSGGGGSGGGGSGGGGSGGGG |
| | | | SGGGGSDKTHTCPPCPAPELLGGPSVFLFPPKPKDTLMISRTPEVTCVVVDVSHEDPE |
| | | | VKFNWYVDGVEVHNAKTKPCEEQYGSTYRCVSVLTVLHQDWLNGKEYKCKVSNKALPA |
| | | | PIEKTISKAKGQPREPQVYTLPPSREEMTKNQVSLTCLVKGFYPSDIAVEWESNGQPE |
| | | | NNYKTTPPVLDSDGSFFLYSKLTVDKSRWQQGNVFSCSVMHEALHNHYTQKSLSLSPG |
| | | | K |
| 2281 | CS | artificial | DIQMTQSPSSLSASVGDRVTITCKASQDVSTAVAWYQQKPGKAPKLLIYSASYRYTGV |
| | PDL241.12 LH | | PDRFTGSGSGTDFTLTISSLQPEDFATYYCQQHYSTPPYTFGCGTKVEIKRGGGGSGG |
| | CC x I2E | | GGSGGGGSQVQLVQSGAEVKKPGASVKVSCKASGYAFSSSWMNWVRQAPGQCLEWIGR |
| | x scFc- | | IYPGDADAKYNAKFKGKATLTADKSTSTAYMELSSLRSEDTAVYYCARSTMIATGAMD |
| | Full | | YWGQGTLVTVSSSGGGGSEVQLVESGGGLVQPGGSLKLSCAASGFTFNKYAINWVRQA |
| | Sequence | | PGKGLEWVARIRSKYNNYATYYADAVKDRFTISRDDSKNTVYLQMNNLKTEDTAVYYC |
| | | | ARAGNFGSSYISYWAYWGQGTLVTVSSGGGGSGGGGSGGGGSQTVVTQEPSLTVSPGG |
| | | | TVTITCGSSTGAVTSGNYPNWVQKKPGQAPRGLIGGTKFLAPGTPARFSGSLSGGKAA |
| | | | LTLSGVQPEDEAEYYCVLWYSNRWVFGSGTKLTVLGGGGDKTHTCPPCPAPELLGGPS |
| | | | VFLFPPKPKDTLMISRTPEVTCVVVDVSHEDPEVKFNWYVDGVEVHNAKTKPCEEQYG |
| | | | STYRCVSVLTVLHQDWLNGKEYKCKVSNKALPAPIEKTISKAKGQPREPQVYTLPPSR |
| | | | EEMTKNQVSLTCLVKGFYPSDIAVEWESNGQPENNYKTTPPVLDSDGSFFLYSKLTVD |
| | | | KSRWQQGNVFSCSVMHEALHNHYTQKSLSLSPGKGGGGSGGGGSGGGGSGGGGSGGGG |
| | | | SGGGGSDKTHTCPPCPAPELLGGPSVFLFPPKPKDTLMISRTPEVTCVVVDVSHEDPE |
| | | | VKFNWYVDGVEVHNAKTKPCEEQYGSTYRCVSVLTVLHQDWLNGKEYKCKVSNKALPA |
| | | | PIEKTISKAKGQPREPQVYTLPPSREEMTKNQVSLTCLVKGFYPSDIAVEWESNGQPE |
| | | | NNYKTTPPVLDSDGSFFLYSKLTVDKSRWQQGNVFSCSVMHEALHNHYTQKSLSLSPG |
| | | | K |
| 2282 | CS | artificial | DIQMTQSPSSLSASVGDRVTITCKASQDVSTAVAWYQQKPGKAPKLLIYSASYRYTGV |
| | PDL241.12 LH | | PDRFTGSGSGTDFTLTISSLQPEDFATYYCQQHYSTPPYTFGCGTKVEIKRGGGGSGG |
| | CC x I2K | | GGSGGGGSQVQLVQSGAEVKKPGASVKVSCKASGYAFSSSWMNWVRQAPGQCLEWIGR |
| | x scFc- | | IYPGDADAKYNAKFKGKATLTADKSTSTAYMELSSLRSEDTAVYYCARSTMIATGAMD |
| | Full | | YWGQGTLVTVSSSGGGGSEVQLVESGGGLVQPGGSLKLSCAASGFTENKYAMNWVRQA |
| | Sequence | | PGKGLEWVARIRSKYNNYATYYAEAVKGRFTISRDDSKNTVYLQMNNLKTEDTAVYYC |
| | | | VRNENIGTSYISYWAYWGQGTLVTVSSGGGGSGGGGSGGGGSQTVVTQEPSLTVSPGG |
| | | | TVTITCGSSTGAVTSGNYPNWVQQKPGQAPRGLIGGTKFLAPGTPARFSGSLSGGKAA |
| | | | LTLSGVQPEDEAEYYCVLWYSNRWVFGSGTKLTVLGGGGDKTHTCPPCPAPELLGGPS |
| | | | VFLFPPKPKDTLMISRTPEVTCVVVDVSHEDPEVKENWYVDGVEVHNAKTKPCEEQYG |
| | | | STYRCVSVLTVLHQDWLNGKEYKCKVSNKALPAPIEKTISKAKGQPREPQVYTLPPSR |
| | | | EEMTKNQVSLTCLVKGFYPSDIAVEWESNGQPENNYKTTPPVLDSDGSFFLYSKLTVD |
| | | | KSRWQQGNVFSCSVMHEALHNHYTQKSLSLSPGKGGGGSGGGGSGGGGSGGGGSGGGG |
| | | | SGGGGSDKTHTCPPCPAPELLGGPSVFLFPPKPKDTLMISRTPEVTCVVVDVSHEDPE |
| | | | VKFNWYVDGVEVHNAKTKPCEEQYGSTYRCVSVLTVLHQDWLNGKEYKCKVSNKALPA |
| | | | PIEKTISKAKGQPREPQVYTLPPSREEMTKNQVSLTCLVKGFYPSDIAVEWESNGQPE |
| | | | NNYKTTPPVLDSDGSFFLYSKLTVDKSRWQQGNVFSCSVMHEALHNHYTQKSLSLSPG |
| | | | K |
| | | | DIQMTQSPSSLSASVGDRVTITCKASQDVSTAVAWYQQKPGKAPKLLIYSASYRYTGV |
| 2283 | CS | artificial | PDRFTGSGSGTDFTLTISSLQPEDFATYYCQQHYSTPPYTFGCGTKVEIKRGGGGSGG |
| | PDL241.12 LH | | GGSGGGGSQVQLVQSGAEVKKPGASVKVSCKASGYAFSSSWMNWVRQAPGQCLEWIGR |
| | CC x I2L | | IYPGDADAKYNAKFKGKATLTADKSTSTAYMELSSLRSEDTAVYYCARSTMIATGAMD |
| | x scFc- | | YWGQGTLVTVSSSGGGGSEVQLVESGGGLVQPGGSLKLSCAASGFTFNKYAMNWVRQA |
| | Full | | PGKGMEWVARIRSKYNNYATYYADAVKDRFTISRDDSKNTLYLQMNNLKTEDTAVYYC |
| | Sequence | | VRAGNFGSSYISYFAYWGQGTLVTVSSGGGGSGGGGSGGGGSQTVVTQEPSLTVSPGG |
| | | | TVTITCGSSTGAVTSGNYPNWIQKKPGQAPRGLIGGTKFLAPGTPARFSGSLEGGKAA |
| | | | LTLSGVQPEDEAEYYCVLYYSNRWVFGSGTKLTVLGGGGDKTHTCPPCPAPELLGGPS |
| | | | VFLFPPKPKDTLMISRTPEVTCVVVDVSHEDPEVKENWYVDGVEVHNAKTKPCEEQYG |
| | | | STYRCVSVLTVLHQDWLNGKEYKCKVSNKALPAPIEKTISKAKGQPREPQVYTLPPSR |
| | | | EEMTKNQVSLTCLVKGFYPSDIAVEWESNGQPENNYKTTPPVLDSDGSFFLYSKLTVD |
| | | | KSRWQQGNVFSCSVMHEALHNHYTQKSLSLSPGKGGGGSGGGGSGGGGSGGGGSGGGG |
| | | | SGGGGSDKTHTCPPCPAPELLGGPSVFLFPPKPKDTLMISRTPEVTCVVVDVSHEDPE |
| | | | VKFNWYVDGVEVHNAKTKPCEEQYGSTYRCVSVLTVLHQDWLNGKEYKCKVSNKALPA |
| | | | PIEKTISKAKGQPREPQVYTLPPSREEMTKNQVSLTCLVKGFYPSDIAVEWESNGQPE |
| | | | NNYKTTPPVLDSDGSFFLYSKLTVDKSRWQQGNVFSCSVMHEALHNHYTQKSLSLSPG |
| | | | K |
| 2284 | CS | artificial | DIQMTQSPSSLSASVGDRVTITCKASQDVSTAVAWYQQKPGKAPKLLIYSASYRYTGV |
| | PDL241.12 LH | | PDRFTGSGSGTDFTLTISSLQPEDFATYYCQQHYSTPPYTFGCGTKVEIKRGGGGSGG |
| | CC x I2M | | GGSGGGGSQVQLVQSGAEVKKPGASVKVSCKASGYAFSSSWMNWVRQAPGQCLEWIGR |
| | x scFc- | | IYPGDADAKYNAKFKGKATLTADKSTSTAYMELSSLRSEDTAVYYCARSTMIATGAMD |
| | Full | | YWGQGTLVTVSSSGGGGSEVQLVESGGGLVQPGGSLKLSCAASGFTFNKYAMNWVRQA |
| | Sequence | | PGKGMEWVARIRSKYNNYATYYADAVKDRFTISRDDSKNTLYLQMNNLKTEDTAVYYC |
| | | | VRAGNFGTSYISYWAYWGQGTLVTVSSGGGGSGGGGSGGGGSQTVVTQEPSLTVSPGG |
| | | | TVTITCGSSTGAVTSGNYPNWVQKKPGQAPRGLIGGTKFLAPGTPARFSGSLSGGKAA |
| | | | LTLSGVQPEDEAEYYCVLWYSNRWVFGSGTKLTVLGGGGDKTHTCPPCPAPELLGGPS |
| | | | VFLFPPKPKDTLMISRTPEVTCVVVDVSHEDPEVKFNWYVDGVEVHNAKTKPCEEQYG |
| | | | STYRCVSVLTVLHQDWLNGKEYKCKVSNKALPAPIEKTISKAKGQPREPQVYTLPPSR |
| | | | EEMTKNQVSLTCLVKGFYPSDIAVEWESNGQPENNYKTTPPVLDSDGSFFLYSKLTVD |
| | | | KSRWQQGNVFSCSVMHEALHNHYTQKSLSLSPGKGGGGSGGGGSGGGGSGGGGSGGGG |
| | | | SGGGGSDKTHTCPPCPAPELLGGPSVFLFPPKPKDTLMISRTPEVTCVVVDVSHEDPE |
| | | | VKFNWYVDGVEVHNAKTKPCEEQYGSTYRCVSVLTVLHQDWLNGKEYKCKVSNKALPA |
| | | | PIEKTISKAKGQPREPQVYTLPPSREEMTKNQVSLTCLVKGFYPSDIAVEWESNGQPE |
| | | | NNYKTTPPVLDSDGSFFLYSKLTVDKSRWQQGNVFSCSVMHEALHNHYTQKSLSLSPG |
| | | | K |
| 2285 | CS | artificial | DIQMTQSPSSLSASVGDRVTITCKASQDVSTAVAWYQQKPGKAPKLLIYSASYRYTGV |
| | PDL241.12 LH | | PDRFTGSGSGTDFTLTISSLQPEDFATYYCQQHYSTPPYTFGCGTKVEIKRGGGGSGG |
| | CC x I2M2 | | GGSGGGGSQVQLVQSGAEVKKPGASVKVSCKASGYAFSSSWMNWVRQAPGQCLEWIGR |
| | x scFc- | | IYPGDADAKYNAKFKGKATLTADKSTSTAYMELSSLRSEDTAVYYCARSTMIATGAMD |
| | Full | | YWGQGTLVTVSSSGGGGSEVQLVESGGGLVQPGGSLKLSCAASGFTENKYAINWVREA |
| | Sequence | | PGKGLEWVARIRSKYNNYATYYADAVKDRFTISRDDSKNTAYLQMNNLKTEDTAVYYC |
| | | | VRNANFGTSYISYFAYWGQGTLVTVSSGGGGSGGGGSGGGGSQTVVTQEPSLTVSPGG |
| | | | TVTLTCGSSTGAVTSGNYPNWVQKKPGQAPRGLIGGTKFLAPGTPARFSGSLLGGKAA |
| | | | LTLSGVQPEDEAEYYCVLWYSNRWVFGSGTKLTVLGGGGDKTHTCPPCPAPELLGGPS |
| | | | VFLFPPKPKDTLMISRTPEVTCVVVDVSHEDPEVKENWYVDGVEVHNAKTKPCEEQYG |
| | | | STYRCVSVLTVLHQDWLNGKEYKCKVSNKALPAPIEKTISKAKGQPREPQVYTLPPSR |
| | | | EEMTKNQVSLTCLVKGFYPSDIAVEWESNGQPENNYKTTPPVLDSDGSFFLYSKLTVD |
| | | | KSRWQQGNVFSCSVMHEALHNHYTQKSLSLSPGKGGGGSGGGGSGGGGSGGGGSGGGG |
| | | | SGGGGSDKTHTCPPCPAPELLGGPSVFLFPPKPKDTLMISRTPEVTCVVVDVSHEDPE |
| | | | VKFNWYVDGVEVHNAKTKPCEEQYGSTYRCVSVLTVLHQDWLNGKEYKCKVSNKALPA |
| | | | PIEKTISKAKGQPREPQVYTLPPSREEMTKNQVSLTCLVKGFYPSDIAVEWESNGQPE |
| | | | NNYKTTPPVLDSDGSFFLYSKLTVDKSRWQQGNVFSCSVMHEALHNHYTQKSLSLSPG |
| | | | K |
| 2286 | CL6 3D4- | artificial | GYYMH |
| | 01.G2 LH- | | |
| | HCDR1 | | |
| 2287 | CL6 3D4- | artificial | WINPNSGETNYAQKFQG |
| | 01.G2 LH- | | |
| | HCDR2 | | |
| 2288 | CL6 3D4- | artificial | DALIVVAPVTRDYYYYGMDV |
| | 01.G2 LH- | | |
| | HCDR3 | | |
| 2289 | CL6 3D4- | artificial | RASQSVSSSYLA |
| | 01.G2 LH- | | |
| | LCDR1 | | |
| 2290 | CL6 3D4- | artificial | GASSRAT |
| | 01.G2 LH- | | |
| | LCDR2 | | |
| 2291 | CL6 3D4- | artificial | QQYGSSPLT |
| | 01.G2 LH- | | |
| | LCDR3 | | |
| 2292 | CL6 3D4- | artificial | QVQLVQSGAEVKKPGASVKVSCKASGYTFTGYYMHWVRQAPGQCLEWMGWINPNSGET |
| | 01.G2 LH- | | NYAQKFQGRVTMTRDTSISTAYMELSRLRSDDTAVYYCARDALIVVAPVTRDYYYYGM |
| | VH | | DVWGQGTTVTVSS |
| 2293 | CL6 3D4- | artificial | EIVLTQSPGTLSLSPGERATLSCRASQSVSSSYLAWYQQKPGQAPRLLIYGASSRATG |
| | 01.G2 LH- | | IPDRFSGSGSGTDFTLTISRLEPEDFAVYYCQQYGSSPLTFGCGTKLEIK |
| | VL | | |
| 2294 | CL6 3D4- | artificial | EIVLTQSPGTLSLSPGERATLSCRASQSVSSSYLAWYQQKPGQAPRLLIYGASSRATG |
| | 01.G2 LH x | | IPDRFSGSGSGTDFTLTISRLEPEDFAVYYCQQYGSSPLTFGCGTKLEIKGGGGSGGG |
| | I2Cx scFc- | | GSGGGGSQVQLVQSGAEVKKPGASVKVSCKASGYTFTGYYMHWVRQAPGQCLEWMGWI |
| | Full | | NPNSGETNYAQKFQGRVTMTRDTSISTAYMELSRLRSDDTAVYYCARDALIVVAPVTR |
| | Sequence | | DYYYYGMDVWGQGTTVTVSSSGGGGSEVQLVESGGGLVQPGGSLKLSCAASGFTENKY |
| | | | AMNWVRQAPGKGLEWVARIRSKYNNYATYYADSVKDRFTISRDDSKNTAYLQMNNLKT |
| | | | EDTAVYYCVRHGNFGNSYISYWAYWGQGTLVTVSSGGGGSGGGGSGGGGSQTVVTQEP |
| | | | SLTVSPGGTVTLTCGSSTGAVTSGNYPNWVQQKPGQAPRGLIGGTKFLAPGTPARFSG |
| | | | SLLGGKAALTLSGVQPEDEAEYYCVLWYSNRWVFGGGTKLTVLGGGGDKTHTCPPCPA |
| | | | PELLGGPSVFLFPPKPKDTLMISRTPEVTCVVVDVSHEDPEVKENWYVDGVEVHNAKT |
| | | | KPCEEQYGSTYRCVSVLTVLHQDWLNGKEYKCKVSNKALPAPIEKTISKAKGQPREPQ |
| | | | VYTLPPSREEMTKNQVSLTCLVKGFYPSDIAVEWESNGQPENNYKTTPPVLDSDGSFF |
| | | | LYSKLTVDKSRWQQGNVFSCSVMHEALHNHYTQKSLSLSPGKGGGGSGGGGSGGGGSG |
| | | | GGGSGGGGSGGGGSDKTHTCPPCPAPELLGGPSVFLFPPKPKDTLMISRTPEVTCVVV |
| | | | DVSHEDPEVKFNWYVDGVEVHNAKTKPCEEQYGSTYRCVSVLTVLHQDWLNGKEYKCK |
| | | | VSNKALPAPIEKTISKAKGQPREPQVYTLPPSREEMTKNQVSLTCLVKGFYPSDIAVE |
| | | | WESNGQPENNYKTTPPVLDSDGSFFLYSKLTVDKSRWQQGNVFSCSVMHEALHNHYTQ |
| | | | KSLSLSPGK |
| 2295 | CL6 3D4- | artificial | EIVLTQSPGTLSLSPGERATLSCRASQSVSSSYLAWYQQKPGQAPRLLIYGASSRATG |
| | 01.G2 LH x | | IPDRFSGSGSGTDFTLTISRLEPEDFAVYYCQQYGSSPLTFGCGTKLEIKGGGGSGGG |
| | 12Ex SCFc- | | GSGGGGSQVQLVQSGAEVKKPGASVKVSCKASGYTFTGYYMHWVRQAPGQCLEWMGWI |
| | Full | | NPNSGETNYAQKFQGRVTMTRDTSISTAYMELSRLRSDDTAVYYCARDALIVVAPVTR |
| | Sequence | | DYYYYGMDVWGQGTTVTVSSSGGGGSEVQLVESGGGLVQPGGSLKLSCAASGFTENKY |
| | | | AINWVRQAPGKGLEWVARIRSKYNNYATYYADAVKDRFTISRDDSKNTVYLQMNNLKT |
| | | | EDTAVYYCARAGNFGSSYISYWAYWGQGTLVTVSSGGGGSGGGGSGGGGSQTVVTQEP |
| | | | SLTVSPGGTVTITCGSSTGAVTSGNYPNWVQKKPGQAPRGLIGGTKFLAPGTPARFSG |
| | | | SLSGGKAALTLSGVQPEDEAEYYCVLWYSNRWVFGSGTKLTVLGGGGDKTHTCPPCPA |
| | | | PELLGGPSVFLFPPKPKDTLMISRTPEVTCVVVDVSHEDPEVKENWYVDGVEVHNAKT |
| | | | KPCEEQYGSTYRCVSVLTVLHQDWLNGKEYKCKVSNKALPAPIEKTISKAKGQPREPQ |
| | | | VYTLPPSREEMTKNQVSLTCLVKGFYPSDIAVEWESNGQPENNYKTTPPVLDSDGSFF |
| | | | LYSKLTVDKSRWQQGNVFSCSVMHEALHNHYTQKSLSLSPGKGGGGSGGGGSGGGGSG |
| | | | GGGSGGGGSGGGGSDKTHTCPPCPAPELLGGPSVFLFPPKPKDTLMISRTPEVTCVVV |
| | | | DVSHEDPEVKFNWYVDGVEVHNAKTKPCEEQYGSTYRCVSVLTVLHQDWLNGKEYKCK |
| | | | VSNKALPAPIEKTISKAKGQPREPQVYTLPPSREEMTKNQVSLTCLVKGFYPSDIAVE |
| | | | WESNGQPENNYKTTPPVLDSDGSFFLYSKLTVDKSRWQQGNVFSCSVMHEALHNHYTQ |
| | | | KSLSLSPGK |
| 2296 | CL6 3D4- | artificial | EIVLTQSPGTLSLSPGERATLSCRASQSVSSSYLAWYQQKPGQAPRLLIYGASSRATG |
| | 01.G2 LH x | | IPDRFSGSGSGTDFTLTISRLEPEDFAVYYCQQYGSSPLTFGCGTKLEIKGGGGSGGG |
| | I2Kx scFc- | | GSGGGGSQVQLVQSGAEVKKPGASVKVSCKASGYTFTGYYMHWVRQAPGQCLEWMGWI |
| | Full | | NPNSGETNYAQKFQGRVTMTRDTSISTAYMELSRLRSDDTAVYYCARDALIVVAPVTR |
| | Sequence | | DYYYYGMDVWGQGTTVTVSSSGGGGSEVQLVESGGGLVQPGGSLKLSCAASGFTENKY |
| | | | AMNWVRQAPGKGLEWVARIRSKYNNYATYYAEAVKGRFTISRDDSKNTVYLQMNNLKT |
| | | | EDTAVYYCVRNENIGTSYISYWAYWGQGTLVTVSSGGGGSGGGGSGGGGSQTVVTQEP |
| | | | SLTVSPGGTVTITCGSSTGAVTSGNYPNWVQQKPGQAPRGLIGGTKFLAPGTPARFSG |
| | | | SLSGGKAALTLSGVQPEDEAEYYCVLWYSNRWVFGSGTKLTVLGGGGDKTHTCPPCPA |
| | | | PELLGGPSVFLFPPKPKDTLMISRTPEVTCVVVDVSHEDPEVKENWYVDGVEVHNAKT |
| | | | KPCEEQYGSTYRCVSVLTVLHQDWLNGKEYKCKVSNKALPAPIEKTISKAKGQPREPQ |
| | | | VYTLPPSREEMTKNQVSLTCLVKGFYPSDIAVEWESNGQPENNYKTTPPVLDSDGSFF |
| | | | LYSKLTVDKSRWQQGNVFSCSVMHEALHNHYTQKSLSLSPGKGGGGSGGGGSGGGGSG |
| | | | GGGSGGGGSGGGGSDKTHTCPPCPAPELLGGPSVFLFPPKPKDTLMISRTPEVTCVVV |
| | | | DVSHEDPEVKFNWYVDGVEVHNAKTKPCEEQYGSTYRCVSVLTVLHQDWLNGKEYKCK |
| | | | VSNKALPAPIEKTISKAKGQPREPQVYTLPPSREEMTKNQVSLTCLVKGFYPSDIAVE |
| | | | WESNGQPENNYKTTPPVLDSDGSFFLYSKLTVDKSRWQQGNVFSCSVMHEALHNHYTQ |
| | | | KSLSLSPGK |
| 2297 | CL6 3D4- | artificial | EIVLTQSPGTLSLSPGERATLSCRASQSVSSSYLAWYQQKPGQAPRLLIYGASSRATG |
| | 01.G2 LH x | | IPDRESGSGSGTDFTLTISRLEPEDFAVYYCQQYGSSPLTFGCGTKLEIKGGGGSGGG |
| | I2Lx scFc- | | GSGGGGSQVQLVQSGAEVKKPGASVKVSCKASGYTFTGYYMHWVRQAPGQCLEWMGWI |
| | Full | | NPNSGETNYAQKFQGRVTMTRDTSISTAYMELSRLRSDDTAVYYCARDALIVVAPVTR |
| | Sequence | | DYYYYGMDVWGQGTTVTVSSSGGGGSEVQLVESGGGLVQPGGSLKLSCAASGFTENKY |
| | | | AMNWVRQAPGKGMEWVARIRSKYNNYATYYADAVKDRFTISRDDSKNTLYLQMNNLKT |
| | | | EDTAVYYCVRAGNFGSSYISYFAYWGQGTLVTVSSGGGGSGGGGSGGGGSQTVVTQEP |
| | | | SLTVSPGGTVTITCGSSTGAVTSGNYPNWIQKKPGQAPRGLIGGTKFLAPGTPARFSG |
| | | | SLEGGKAALTLSGVQPEDEAEYYCVLYYSNRWVFGSGTKLTVLGGGGDKTHTCPPCPA |
| | | | PELLGGPSVFLFPPKPKDTLMISRTPEVTCVVVDVSHEDPEVKENWYVDGVEVHNAKT |
| | | | KPCEEQYGSTYRCVSVLTVLHQDWLNGKEYKCKVSNKALPAPIEKTISKAKGQPREPQ |
| | | | VYTLPPSREEMTKNQVSLTCLVKGFYPSDIAVEWESNGQPENNYKTTPPVLDSDGSFF |
| | | | LYSKLTVDKSRWQQGNVFSCSVMHEALHNHYTQKSLSLSPGKGGGGSGGGGSGGGGSG |
| | | | GGGSGGGGSGGGGSDKTHTCPPCPAPELLGGPSVFLFPPKPKDTLMISRTPEVTCVVV |
| | | | DVSHEDPEVKFNWYVDGVEVHNAKTKPCEEQYGSTYRCVSVLTVLHQDWLNGKEYKCK |
| | | | VSNKALPAPIEKTISKAKGQPREPQVYTLPPSREEMTKNQVSLTCLVKGFYPSDIAVE |
| | | | WESNGQPENNYKTTPPVLDSDGSFFLYSKLTVDKSRWQQGNVFSCSVMHEALHNHYTQ |
| | | | KSLSLSPGK |
| 2298 | CL6 3D4- | artificial | EIVLTQSPGTLSLSPGERATLSCRASQSVSSSYLAWYQQKPGQAPRLLIYGASSRATG |
| | 01.G2 LH x | | IPDRFSGSGSGTDFTLTISRLEPEDFAVYYCQQYGSSPLTFGCGTKLEIKGGGGSGGG |
| | I2M2x scFc | | GSGGGGSQVQLVQSGAEVKKPGASVKVSCKASGYTFTGYYMHWVRQAPGQCLEWMGWI |
| | -Full | | NPNSGETNYAQKFQGRVTMTRDTSISTAYMELSRLRSDDTAVYYCARDALIVVAPVTR |
| | Sequence | | DYYYYGMDVWGQGTTVTVSSSGGGGSEVQLVESGGGLVQPGGSLKLSCAASGFTENKY |
| | | | AINWVREAPGKGLEWVARIRSKYNNYATYYADAVKDRFTISRDDSKNTAYLQMNNLKT |
| | | | EDTAVYYCVRNANFGTSYISYFAYWGQGTLVTVSSGGGGSGGGGSGGGGSQTVVTQEP |
| | | | SLTVSPGGTVTLTCGSSTGAVTSGNYPNWVQKKPGQAPRGLIGGTKFLAPGTPARFSG |
| | | | SLLGGKAALTLSGVQPEDEAEYYCVLWYSNRWVFGSGTKLTVLGGGGDKTHTCPPCPA |
| | | | PELLGGPSVFLFPPKPKDTLMISRTPEVTCVVVDVSHEDPEVKENWYVDGVEVHNAKT |
| | | | KPCEEQYGSTYRCVSVLTVLHQDWLNGKEYKCKVSNKALPAPIEKTISKAKGQPREPQ |
| | | | VYTLPPSREEMTKNQVSLTCLVKGFYPSDIAVEWESNGQPENNYKTTPPVLDSDGSFF |
| | | | LYSKLTVDKSRWQQGNVFSCSVMHEALHNHYTQKSLSLSPGKGGGGSGGGGSGGGGSG |
| | | | GGGSGGGGSGGGGSDKTHTCPPCPAPELLGGPSVFLFPPKPKDTLMISRTPEVTCVVV |
| | | | DVSHEDPEVKFNWYVDGVEVHNAKTKPCEEQYGSTYRCVSVLTVLHQDWLNGKEYKCK |
| | | | VSNKALPAPIEKTISKAKGQPREPQVYTLPPSREEMTKNQVSLTCLVKGFYPSDIAVE |
| | | | WESNGQPENNYKTTPPVLDSDGSFFLYSKLTVDKSRWQQGNVFSCSVMHEALHNHYTQ |
| | | | KSLSLSPGK |
| 2299 | CL6 3D4- | artificial | EIVLTQSPGTLSLSPGERATLSCRASQSVSSSYLAWYQQKPGQAPRLLIYGASSRATG |
| | 01.G2 LH x | | IPDRFSGSGSGTDFTLTISRLEPEDFAVYYCQQYGSSPLTFGCGTKLEIKGGGGSGGG |
| | I2Mx scFc- | | GSGGGGSQVQLVQSGAEVKKPGASVKVSCKASGYTFTGYYMHWVRQAPGQCLEWMGWI |
| | Full | | NPNSGETNYAQKFQGRVTMTRDTSISTAYMELSRLRSDDTAVYYCARDALIVVAPVTR |
| | Sequence | | DYYYYGMDVWGQGTTVTVSSSGGGGSEVQLVESGGGLVQPGGSLKLSCAASGFTENKY |
| | | | AMNWVRQAPGKGMEWVARIRSKYNNYATYYADAVKDRFTISRDDSKNTLYLQMNNLKT |
| | | | EDTAVYYCVRAGNFGTSYISYWAYWGQGTLVTVSSGGGGSGGGGSGGGGSQTVVTQEP |
| | | | SLTVSPGGTVTITCGSSTGAVTSGNYPNWVQKKPGQAPRGLIGGTKFLAPGTPARFSG |
| | | | SLSGGKAALTLSGVQPEDEAEYYCVLWYSNRWVFGSGTKLTVLGGGGDKTHTCPPCPA |
| | | | PELLGGPSVFLFPPKPKDTLMISRTPEVTCVVVDVSHEDPEVKENWYVDGVEVHNAKT |
| | | | KPCEEQYGSTYRCVSVLTVLHQDWLNGKEYKCKVSNKALPAPIEKTISKAKGQPREPQ |
| | | | VYTLPPSREEMTKNQVSLTCLVKGFYPSDIAVEWESNGQPENNYKTTPPVLDSDGSFF |
| | | | LYSKLTVDKSRWQQGNVFSCSVMHEALHNHYTQKSLSLSPGKGGGGSGGGGSGGGGSG |
| | | | GGGSGGGGSGGGGSDKTHTCPPCPAPELLGGPSVFLFPPKPKDTLMISRTPEVTCVVV |
| | | | DVSHEDPEVKFNWYVDGVEVHNAKTKPCEEQYGSTYRCVSVLTVLHQDWLNGKEYKCK |
| | | | VSNKALPAPIEKTISKAKGQPREPQVYTLPPSREEMTKNQVSLTCLVKGFYPSDIAVE |
| | | | WESNGQPENNYKTTPPVLDSDGSFFLYSKLTVDKSRWQQGNVFSCSVMHEALHNHYTQ |
| | | | KSLSLSPGK |
| 2300 | CL 10D8 CC | artificial | GYYMH |
| | -HCDR1 | | |
| 2301 | CL 10D8 CC | artificial | WINPNSGGTKYAQKFQG |
| | -HCDR2 | | |
| 2302 | CL 10D8 CC | artificial | DRITVAGTYYYYGMDV |
| | -HCDR3 | | |
| 2303 | CL 10D8 CC | artificial | RASQGVNNWLA |
| | -LCDR1 | | |
| 2304 | CL 10D8 CC | artificial | TASSLQS |
| | -LCDR2 | | |
| 2305 | CL 10D8 CC | artificial | QQANSFPIT |
| | -LCDR3 | | |
| 2306 | CL 10D8 CC | artificial | QVQLVQSGAEVKKPGASVKVSCKASGYTFTGYYMHWVRQAPGQCLEWMGWINPNSGGT |
| | -VH | | KYAQKFQGRVTMTRDTSISTAYMELSRLRSDDTAVYYCARDRITVAGTYYYYGMDVWG |
| | | | QGTTVTVSS |
| 2307 | CL 10D8 CC | artificial | DIQMTQSPSSVSASVGDRVTITCRASQGVNNWLAWYQQKPGKAPKLLIYTASSLQSGV |
| | -VL | | PSRFSGSGSGTDFTLTIRSLQPEDFATYYCQQANSFPITFGCGTRLEIK |
| 2308 | CL 10D8 CC | artificial | EIQMTQSPSSVSASVGDRVTITCRASQGVNNWLAWYQQKPGKAPKLLIYTASSLQSGV |
| | EI GQ-VL | | PSRFSGSGSGTDFTLTIRSLQPEDFATYYCQQANSFPITFGCGTRLEIK |
| 2309 | CL 10D8 CC | artificial | QVQLVQSGAEVKKPGASVKVSCKASGYTFTGYYMHWVRQAPGQCLEWMGWINPNSGGT |
| | x I2C x | | KYAQKFQGRVTMTRDTSISTAYMELSRLRSDDTAVYYCARDRITVAGTYYYYGMDVWG |
| | scFc-Full | | QGTTVTVSSGGGGSGGGGSGGGGSDIQMTQSPSSVSASVGDRVTITCRASQGVNNWLA |
| | Sequence | | WYQQKPGKAPKLLIYTASSLQSGVPSRFSGSGSGTDFTLTIRSLQPEDFATYYCQQAN |
| | | | SFPITFGCGTRLEIKSGGGGSEVQLVESGGGLVQPGGSLKLSCAASGFTENKYAMNWV |
| | | | RQAPGKGLEWVARIRSKYNNYATYYADSVKDRFTISRDDSKNTAYLQMNNLKTEDTAV |
| | | | YYCVRHGNFGNSYISYWAYWGQGTLVTVSSGGGGSGGGGSGGGGSQTVVTQEPSLTVS |
| | | | PGGTVTLTCGSSTGAVTSGNYPNWVQQKPGQAPRGLIGGTKFLAPGTPARFSGSLLGG |
| | | | KAALTLSGVQPEDEAEYYCVLWYSNRWVFGGGTKLTVLGGGGDKTHTCPPCPAPELLG |
| | | | GPSVFLFPPKPKDTLMISRTPEVTCVVVDVSHEDPEVKFNWYVDGVEVHNAKTKPCEE |
| | | | QYGSTYRCVSVLTVLHQDWLNGKEYKCKVSNKALPAPIEKTISKAKGQPREPQVYTLP |
| | | | PSREEMTKNQVSLTCLVKGFYPSDIAVEWESNGQPENNYKTTPPVLDSDGSFFLYSKL |
| | | | TVDKSRWQQGNVFSCSVMHEALHNHYTQKSLSLSPGKGGGGSGGGGSGGGGSGGGGSG |
| | | | GGGSGGGGSDKTHTCPPCPAPELLGGPSVFLFPPKPKDTLMISRTPEVTCVVVDVSHE |
| | | | DPEVKFNWYVDGVEVHNAKTKPCEEQYGSTYRCVSVLTVLHQDWLNGKEYKCKVSNKA |
| | | | LPAPIEKTISKAKGQPREPQVYTLPPSREEMTKNQVSLTCLVKGFYPSDIAVEWESNG |
| | | | QPENNYKTTPPVLDSDGSFFLYSKLTVDKSRWQQGNVFSCSVMHEALHNHYTQKSLSL |
| | | | SPGK |
| 2310 | CL 10D8 CC | artificial | QVQLVQSGAEVKKPGASVKVSCKASGYTFTGYYMHWVRQAPGQCLEWMGWINPNSGGT |
| | x I2E x | | KYAQKFQGRVTMTRDTSISTAYMELSRLRSDDTAVYYCARDRITVAGTYYYYGMDVWG |
| | scFc-Full | | QGTTVTVSSGGGGSGGGGSGGGGSDIQMTQSPSSVSASVGDRVTITCRASQGVNNWLA |
| | Sequence | | WYQQKPGKAPKLLIYTASSLQSGVPSRFSGSGSGTDFTLTIRSLQPEDFATYYCQQAN |
| | | | SFPITFGCGTRLEIKSGGGGSEVQLVESGGGLVQPGGSLKLSCAASGFTENKYAINWV |
| | | | RQAPGKGLEWVARIRSKYNNYATYYADAVKDRFTISRDDSKNTVYLQMNNLKTEDTAV |
| | | | YYCARAGNFGSSYISYWAYWGQGTLVTVSSGGGGSGGGGSGGGGSQTVVTQEPSLTVS |
| | | | PGGTVTITCGSSTGAVTSGNYPNWVQKKPGQAPRGLIGGTKFLAPGTPARFSGSLSGG |
| | | | KAALTLSGVQPEDEAEYYCVLWYSNRWVFGSGTKLTVLGGGGDKTHTCPPCPAPELLG |
| | | | GPSVFLFPPKPKDTLMISRTPEVTCVVVDVSHEDPEVKENWYVDGVEVHNAKTKPCEE |
| | | | QYGSTYRCVSVLTVLHQDWLNGKEYKCKVSNKALPAPIEKTISKAKGQPREPQVYTLP |
| | | | PSREEMTKNQVSLTCLVKGFYPSDIAVEWESNGQPENNYKTTPPVLDSDGSFFLYSKL |
| | | | TVDKSRWQQGNVFSCSVMHEALHNHYTQKSLSLSPGKGGGGSGGGGSGGGGSGGGGSG |
| | | | GGGSGGGGSDKTHTCPPCPAPELLGGPSVFLFPPKPKDTLMISRTPEVTCVVVDVSHE |
| | | | DPEVKFNWYVDGVEVHNAKTKPCEEQYGSTYRCVSVLTVLHQDWLNGKEYKCKVSNKA |
| | | | LPAPIEKTISKAKGQPREPQVYTLPPSREEMTKNQVSLTCLVKGFYPSDIAVEWESNG |
| | | | QPENNYKTTPPVLDSDGSFFLYSKLTVDKSRWQQGNVFSCSVMHEALHNHYTQKSLSL |
| | | | SPGK |
| 2311 | CL 10D8 CC | artificial | QVQLVQSGAEVKKPGASVKVSCKASGYTFTGYYMHWVRQAPGQCLEWMGWINPNSGGT |
| | x I2K x | | KYAQKFQGRVTMTRDTSISTAYMELSRLRSDDTAVYYCARDRITVAGTYYYYGMDVWG |
| | scFc-Full | | QGTTVTVSSGGGGSGGGGSGGGGSDIQMTQSPSSVSASVGDRVTITCRASQGVNNWLA |
| | Sequence | | WYQQKPGKAPKLLIYTASSLQSGVPSRFSGSGSGTDFTLTIRSLQPEDFATYYCQQAN |
| | | | SFPITFGCGTRLEIKSGGGGSEVQLVESGGGLVQPGGSLKLSCAASGFTFNKYAMNWV |
| | | | RQAPGKGLEWVARIRSKYNNYATYYAEAVKGRFTISRDDSKNTVYLQMNNLKTEDTAV |
| | | | YYCVRNENIGTSYISYWAYWGQGTLVTVSSGGGGSGGGGSGGGGSQTVVTQEPSLTVS |
| | | | PGGTVTITCGSSTGAVTSGNYPNWVQQKPGQAPRGLIGGTKFLAPGTPARFSGSLSGG |
| | | | KAALTLSGVQPEDEAEYYCVLWYSNRWVFGSGTKLTVLGGGGDKTHTCPPCPAPELLG |
| | | | GPSVFLFPPKPKDTLMISRTPEVTCVVVDVSHEDPEVKFNWYVDGVEVHNAKTKPCEE |
| | | | QYGSTYRCVSVLTVLHQDWLNGKEYKCKVSNKALPAPIEKTISKAKGQPREPQVYTLP |
| | | | PSREEMTKNQVSLTCLVKGFYPSDIAVEWESNGQPENNYKTTPPVLDSDGSFFLYSKL |
| | | | TVDKSRWQQGNVFSCSVMHEALHNHYTQKSLSLSPGKGGGGSGGGGSGGGGSGGGGSG |
| | | | GGGSGGGGSDKTHTCPPCPAPELLGGPSVFLFPPKPKDTLMISRTPEVTCVVVDVSHE |
| | | | DPEVKFNWYVDGVEVHNAKTKPCEEQYGSTYRCVSVLTVLHQDWLNGKEYKCKVSNKA |
| | | | LPAPIEKTISKAKGQPREPQVYTLPPSREEMTKNQVSLTCLVKGFYPSDIAVEWESNG |
| | | | QPENNYKTTPPVLDSDGSFFLYSKLTVDKSRWQQGNVFSCSVMHEALHNHYTQKSLSL |
| | | | SPGK |
| 2312 | CL 10D8 CC | artificial | QVQLVQSGAEVKKPGASVKVSCKASGYTFTGYYMHWVRQAPGQCLEWMGWINPNSGGT |
| | x I2L x | | KYAQKFQGRVTMTRDTSISTAYMELSRLRSDDTAVYYCARDRITVAGTYYYYGMDVWG |
| | scFc-Full | | QGTTVTVSSGGGGSGGGGSGGGGSDIQMTQSPSSVSASVGDRVTITCRASQGVNNWLA |
| | Sequence | | WYQQKPGKAPKLLIYTASSLQSGVPSRFSGSGSGTDFTLTIRSLQPEDFATYYCQQAN |
| | | | SFPITFGCGTRLEIKSGGGGSEVQLVESGGGLVQPGGSLKLSCAASGFTENKYAMNWV |
| | | | RQAPGKGMEWVARIRSKYNNYATYYADAVKDRFTISRDDSKNTLYLQMNNLKTEDTAV |
| | | | YYCVRAGNFGSSYISYFAYWGQGTLVTVSSGGGGSGGGGSGGGGSQTVVTQEPSLTVS |
| | | | PGGTVTITCGSSTGAVTSGNYPNWIQKKPGQAPRGLIGGTKFLAPGTPARFSGSLEGG |
| | | | KAALTLSGVQPEDEAEYYCVLYYSNRWVFGSGTKLTVLGGGGDKTHTCPPCPAPELLG |
| | | | GPSVFLFPPKPKDTLMISRTPEVTCVVVDVSHEDPEVKENWYVDGVEVHNAKTKPCEE |
| | | | QYGSTYRCVSVLTVLHQDWLNGKEYKCKVSNKALPAPIEKTISKAKGQPREPQVYTLP |
| | | | PSREEMTKNQVSLTCLVKGFYPSDIAVEWESNGQPENNYKTTPPVLDSDGSFFLYSKL |
| | | | TVDKSRWQQGNVFSCSVMHEALHNHYTQKSLSLSPGKGGGGSGGGGSGGGGSGGGGSG |
| | | | GGGSGGGGSDKTHTCPPCPAPELLGGPSVFLFPPKPKDTLMISRTPEVTCVVVDVSHE |
| | | | DPEVKFNWYVDGVEVHNAKTKPCEEQYGSTYRCVSVLTVLHQDWLNGKEYKCKVSNKA |
| | | | LPAPIEKTISKAKGQPREPQVYTLPPSREEMTKNQVSLTCLVKGFYPSDIAVEWESNG |
| | | | QPENNYKTTPPVLDSDGSFFLYSKLTVDKSRWQQGNVFSCSVMHEALHNHYTQKSLSL |
| | | | SPGK |
| 2313 | CL 10D8 CC | artificial | QVQLVQSGAEVKKPGASVKVSCKASGYTFTGYYMHWVRQAPGQCLEWMGWINPNSGGT |
| | x I2M x | | KYAQKFQGRVTMTRDTSISTAYMELSRLRSDDTAVYYCARDRITVAGTYYYYGMDVWG |
| | scFc-Full | | QGTTVTVSSGGGGSGGGGSGGGGSDIQMTQSPSSVSASVGDRVTITCRASQGVNNWLA |
| | Sequence | | WYQQKPGKAPKLLIYTASSLQSGVPSRFSGSGSGTDFTLTIRSLQPEDFATYYCQQAN |
| | | | SFPITFGCGTRLEIKSGGGGSEVQLVESGGGLVQPGGSLKLSCAASGFTENKYAMNWV |
| | | | RQAPGKGMEWVARIRSKYNNYATYYADAVKDRFTISRDDSKNTLYLQMNNLKTEDTAV |
| | | | YYCVRAGNFGTSYISYWAYWGQGTLVTVSSGGGGSGGGGSGGGGSQTVVTQEPSLTVS |
| | | | PGGTVTITCGSSTGAVTSGNYPNWVQKKPGQAPRGLIGGTKFLAPGTPARFSGSLSGG |
| | | | KAALTLSGVQPEDEAEYYCVLWYSNRWVFGSGTKLTVLGGGGDKTHTCPPCPAPELLG |
| | | | GPSVFLFPPKPKDTLMISRTPEVTCVVVDVSHEDPEVKENWYVDGVEVHNAKTKPCEE |
| | | | QYGSTYRCVSVLTVLHQDWLNGKEYKCKVSNKALPAPIEKTISKAKGQPREPQVYTLP |
| | | | PSREEMTKNQVSLTCLVKGFYPSDIAVEWESNGQPENNYKTTPPVLDSDGSFFLYSKL |
| | | | TVDKSRWQQGNVFSCSVMHEALHNHYTQKSLSLSPGKGGGGSGGGGSGGGGSGGGGSG |
| | | | GGGSGGGGSDKTHTCPPCPAPELLGGPSVFLFPPKPKDTLMISRTPEVTCVVVDVSHE |
| | | | DPEVKFNWYVDGVEVHNAKTKPCEEQYGSTYRCVSVLTVLHQDWLNGKEYKCKVSNKA |
| | | | LPAPIEKTISKAKGQPREPQVYTLPPSREEMTKNQVSLTCLVKGFYPSDIAVEWESNG |
| | | | QPENNYKTTPPVLDSDGSFFLYSKLTVDKSRWQQGNVFSCSVMHEALHNHYTQKSLSL |
| | | | SPGK |
| 2314 | CL 10D8 CC | artificial | QVQLVQSGAEVKKPGASVKVSCKASGYTFTGYYMHWVRQAPGQCLEWMGWINPNSGGT |
| | x I2M2 x | | KYAQKFQGRVTMTRDTSISTAYMELSRLRSDDTAVYYCARDRITVAGTYYYYGMDVWG |
| | scFc-Full | | QGTTVTVSSGGGGSGGGGSGGGGSDIQMTQSPSSVSASVGDRVTITCRASQGVNNWLA |
| | Sequence | | WYQQKPGKAPKLLIYTASSLQSGVPSRFSGSGSGTDFTLTIRSLQPEDFATYYCQQAN |
| | | | SFPITFGCGTRLEIKSGGGGSEVQLVESGGGLVQPGGSLKLSCAASGFTENKYAINWV |
| | | | REAPGKGLEWVARIRSKYNNYATYYADAVKDRFTISRDDSKNTAYLQMNNLKTEDTAV |
| | | | YYCVRNANFGTSYISYFAYWGQGTLVTVSSGGGGSGGGGSGGGGSQTVVTQEPSLTVS |
| | | | PGGTVTLTCGSSTGAVTSGNYPNWVQKKPGQAPRGLIGGTKFLAPGTPARFSGSLLGG |
| | | | KAALTLSGVQPEDEAEYYCVLWYSNRWVFGSGTKLTVLGGGGDKTHTCPPCPAPELLG |
| | | | GPSVFLFPPKPKDTLMISRTPEVTCVVVDVSHEDPEVKFNWYVDGVEVHNAKTKPCEE |
| | | | QYGSTYRCVSVLTVLHQDWLNGKEYKCKVSNKALPAPIEKTISKAKGQPREPQVYTLP |
| | | | PSREEMTKNQVSLTCLVKGFYPSDIAVEWESNGQPENNYKTTPPVLDSDGSFFLYSKL |
| | | | TVDKSRWQQGNVFSCSVMHEALHNHYTQKSLSLSPGKGGGGSGGGGSGGGGSGGGGSG |
| | | | GGGSGGGGSDKTHTCPPCPAPELLGGPSVFLFPPKPKDTLMISRTPEVTCVVVDVSHE |
| | | | DPEVKFNWYVDGVEVHNAKTKPCEEQYGSTYRCVSVLTVLHQDWLNGKEYKCKVSNKA |
| | | | LPAPIEKTISKAKGQPREPQVYTLPPSREEMTKNQVSLTCLVKGFYPSDIAVEWESNG |
| | | | QPENNYKTTPPVLDSDGSFFLYSKLTVDKSRWQQGNVFSCSVMHEALHNHYTQKSLSL |
| | | | SPGK |
| 2315 | DL 8-A7 CC | artificial | SYYWS |
| | -HCDR1 | | |
| 2316 | DL 8-A7 CC | artificial | YVYYSGTTNYNPSLKS |
| | -HCDR2 | | |
| 2317 | DL 8-A7 CC | artificial | IAVTGFYFDY |
| | -HCDR3 | | |
| 2318 | DL 8-A7 CC | artificial | RASQRVNNNYLA |
| | -LCDR1 | | |
| 2319 | DL 8-A7 CC | artificial | GASSRAT |
| | -LCDR2 | | |
| 2320 | DL 8-A7 CC | artificial | QQYDRSPLT |
| | -LCDR3 | | |
| 2321 | DL 8-A7 CC | artificial | QVQLQESGPGLVKPSETLSLTCTVSGGSISSYYWSWIRQPPGKCLEWIGYVYYSGTTN |
| | -VH | | YNPSLKSRVTISVDTSKNQFSLKLSSVTAADTAVYYCASIAVTGFYFDYWGQGTLVTV |
| | | | SS |
| 2322 | DL 8-A7 CC | artificial | EIVLTQSPGTLSLSPGERVTLSCRASQRVNNNYLAWYQQRPGQAPRLLIYGASSRATG |
| | -VL | | IPDRESGSGSGTDFTLTISRLEPEDFAVYYCQQYDRSPLTFGCGTKLEIK |
| 2323 | DL 8-A7 CC | artificial | QVQLQESGPGLVKPSETLSLTCTVSGGSISSYYWSWIRQPPGKCLEWIGYVYYSGTTN |
| | x I2C x | | YNPSLKSRVTISVDTSKNQFSLKLSSVTAADTAVYYCASIAVTGFYFDYWGQGTLVTV |
| | SCFc-Full | | SSGGGGSGGGGSGGGGSEIVLTQSPGTLSLSPGERVTLSCRASQRVNNNYLAWYQQRP |
| | Sequence | | GQAPRLLIYGASSRATGIPDRESGSGSGTDFTLTISRLEPEDFAVYYCQQYDRSPLTF |
| | | | GCGTKLEIKSGGGGSEVQLVESGGGLVQPGGSLKLSCAASGFTENKYAMNWVRQAPGK |
| | | | GLEWVARIRSKYNNYATYYADSVKDRFTISRDDSKNTAYLQMNNLKTEDTAVYYCVRH |
| | | | GNFGNSYISYWAYWGQGTLVTVSSGGGGSGGGGSGGGGSQTVVTQEPSLTVSPGGTVT |
| | | | LTCGSSTGAVTSGNYPNWVQQKPGQAPRGLIGGTKFLAPGTPARFSGSLLGGKAALTL |
| | | | SGVQPEDEAEYYCVLWYSNRWVFGGGTKLTVLGGGGDKTHTCPPCPAPELLGGPSVFL |
| | | | FPPKPKDTLMISRTPEVTCVVVDVSHEDPEVKFNWYVDGVEVHNAKTKPCEEQYGSTY |
| | | | RCVSVLTVLHQDWLNGKEYKCKVSNKALPAPIEKTISKAKGQPREPQVYTLPPSREEM |
| | | | TKNQVSLTCLVKGFYPSDIAVEWESNGQPENNYKTTPPVLDSDGSFFLYSKLTVDKSR |
| | | | WQQGNVFSCSVMHEALHNHYTQKSLSLSPGGGGSGGGGSGGGGSGGGGSGGGGSGGGG |
| | | | SDKTHTCPPCPAPELLGGPSVFLFPPKPKDTLMISRTPEVTCVVVDVSHEDPEVKENW |
| | | | YVDGVEVHNAKTKPCEEQYGSTYRCVSVLTVLHQDWLNGKEYKCKVSNKALPAPIEKT |
| | | | ISKAKGQPREPQVYTLPPSREEMTKNQVSLTCLVKGFYPSDIAVEWESNGQPENNYKT |
| | | | TPPVLDSDGSFFLYSKLTVDKSRWQQGNVFSCSVMHEALHNHYTQKSLSLSPGK |
| 2324 | DL 8-A7 CC | artificial | QVQLQESGPGLVKPSETLSLTCTVSGGSISSYYWSWIRQPPGKCLEWIGYVYYSGTTN |
| | x I2E x | | YNPSLKSRVTISVDTSKNQFSLKLSSVTAADTAVYYCASIAVTGFYFDYWGQGTLVTV |
| | scFc-Full | | SSGGGGSGGGGSGGGGSEIVLTQSPGTLSLSPGERVTLSCRASQRVNNNYLAWYQQRP |
| | Sequence | | GQAPRLLIYGASSRATGIPDRFSGSGSGTDFTLTISRLEPEDFAVYYCQQYDRSPLTF |
| | | | GCGTKLEIKSGGGGSEVQLVESGGGLVQPGGSLKLSCAASGFTFNKYAINWVRQAPGK |
| | | | GLEWVARIRSKYNNYATYYADAVKDRFTISRDDSKNTVYLQMNNLKTEDTAVYYCARA |
| | | | GNFGSSYISYWAYWGQGTLVTVSSGGGGSGGGGSGGGGSQTVVTQEPSLTVSPGGTVT |
| | | | ITCGSSTGAVTSGNYPNWVQKKPGQAPRGLIGGTKFLAPGTPARFSGSLSGGKAALTL |
| | | | SGVQPEDEAEYYCVLWYSNRWVFGSGTKLTVLGGGGDKTHTCPPCPAPELLGGPSVFL |
| | | | FPPKPKDTLMISRTPEVTCVVVDVSHEDPEVKFNWYVDGVEVHNAKTKPCEEQYGSTY |
| | | | RCVSVLTVLHQDWLNGKEYKCKVSNKALPAPIEKTISKAKGQPREPQVYTLPPSREEM |
| | | | TKNQVSLTCLVKGFYPSDIAVEWESNGQPENNYKTTPPVLDSDGSFFLYSKLTVDKSR |
| | | | WQQGNVFSCSVMHEALHNHYTQKSLSLSPGGGGSGGGGSGGGGSGGGGSGGGGSGGGG |
| | | | SDKTHTCPPCPAPELLGGPSVFLFPPKPKDTLMISRTPEVTCVVVDVSHEDPEVKENW |
| | | | YVDGVEVHNAKTKPCEEQYGSTYRCVSVLTVLHQDWLNGKEYKCKVSNKALPAPIEKT |
| | | | ISKAKGQPREPQVYTLPPSREEMTKNQVSLTCLVKGFYPSDIAVEWESNGQPENNYKT |
| | | | TPPVLDSDGSFFLYSKLTVDKSRWQQGNVFSCSVMHEALHNHYTQKSLSLSPGK |
| 2325 | DL 8-A7 CC | artificial | QVQLQESGPGLVKPSETLSLTCTVSGGSISSYYWSWIRQPPGKCLEWIGYVYYSGTTN |
| | x I2K x | | YNPSLKSRVTISVDTSKNQFSLKLSSVTAADTAVYYCASIAVTGFYFDYWGQGTLVTV |
| | ScFc-Full | | SSGGGGSGGGGSGGGGSEIVLTQSPGTLSLSPGERVTLSCRASQRVNNNYLAWYQQRP |
| | Sequence | | GQAPRLLIYGASSRATGIPDRFSGSGSGTDFTLTISRLEPEDFAVYYCQQYDRSPLTF |
| | | | GCGTKLEIKSGGGGSEVQLVESGGGLVQPGGSLKLSCAASGFTENKYAMNWVRQAPGK |
| | | | GLEWVARIRSKYNNYATYYAEAVKGRFTISRDDSKNTVYLQMNNLKTEDTAVYYCVRN |
| | | | ENIGTSYISYWAYWGQGTLVTVSSGGGGSGGGGSGGGGSQTVVTQEPSLTVSPGGTVT |
| | | | ITCGSSTGAVTSGNYPNWVQQKPGQAPRGLIGGTKFLAPGTPARFSGSLSGGKAALTL |
| | | | SGVQPEDEAEYYCVLWYSNRWVFGSGTKLTVLGGGGDKTHTCPPCPAPELLGGPSVEL |
| | | | FPPKPKDTLMISRTPEVTCVVVDVSHEDPEVKFNWYVDGVEVHNAKTKPCEEQYGSTY |
| | | | RCVSVLTVLHQDWLNGKEYKCKVSNKALPAPIEKTISKAKGQPREPQVYTLPPSREEM |
| | | | TKNQVSLTCLVKGFYPSDIAVEWESNGQPENNYKTTPPVLDSDGSFFLYSKLTVDKSR |
| | | | WQQGNVFSCSVMHEALHNHYTQKSLSLSPGGGGSGGGGSGGGGSGGGGSGGGGSGGGG |
| | | | SDKTHTCPPCPAPELLGGPSVFLFPPKPKDTLMISRTPEVTCVVVDVSHEDPEVKENW |
| | | | YVDGVEVHNAKTKPCEEQYGSTYRCVSVLTVLHQDWLNGKEYKCKVSNKALPAPIEKT |
| | | | ISKAKGQPREPQVYTLPPSREEMTKNQVSLTCLVKGFYPSDIAVEWESNGQPENNYKT |
| | | | TPPVLDSDGSFFLYSKLTVDKSRWQQGNVFSCSVMHEALHNHYTQKSLSLSPGK |
| 2326 | DL 8-A7 CC | artificial | QVQLQESGPGLVKPSETLSLTCTVSGGSISSYYWSWIRQPPGKCLEWIGYVYYSGTTN |
| | x I2L x | | YNPSLKSRVTISVDTSKNQFSLKLSSVTAADTAVYYCASIAVTGFYFDYWGQGTLVTV |
| | scFc-Full | | SSGGGGSGGGGSGGGGSEIVLTQSPGTLSLSPGERVTLSCRASQRVNNNYLAWYQQRP |
| | Sequence | | GQAPRLLIYGASSRATGIPDRESGSGSGTDFTLTISRLEPEDFAVYYCQQYDRSPLTF |
| | | | GCGTKLEIKSGGGGSEVQLVESGGGLVQPGGSLKLSCAASGFTFNKYAMNWVRQAPGK |
| | | | GMEWVARIRSKYNNYATYYADAVKDRFTISRDDSKNTLYLQMNNLKTEDTAVYYCVRA |
| | | | GNFGSSYISYFAYWGQGTLVTVSSGGGGSGGGGSGGGGSQTVVTQEPSLTVSPGGTVT |
| | | | ITCGSSTGAVTSGNYPNWIQKKPGQAPRGLIGGTKFLAPGTPARFSGSLEGGKAALTL |
| | | | SGVQPEDEAEYYCVLYYSNRWVFGSGTKLTVLGGGGDKTHTCPPCPAPELLGGPSVFL |
| | | | FPPKPKDTLMISRTPEVTCVVVDVSHEDPEVKFNWYVDGVEVHNAKTKPCEEQYGSTY |
| | | | RCVSVLTVLHQDWLNGKEYKCKVSNKALPAPIEKTISKAKGQPREPQVYTLPPSREEM |
| | | | TKNQVSLTCLVKGFYPSDIAVEWESNGQPENNYKTTPPVLDSDGSFFLYSKLTVDKSR |
| | | | WQQGNVFSCSVMHEALHNHYTQKSLSLSPGGGGSGGGGSGGGGSGGGGSGGGGSGGGG |
| | | | SDKTHTCPPCPAPELLGGPSVFLFPPKPKDTLMISRTPEVTCVVVDVSHEDPEVKENW |
| | | | YVDGVEVHNAKTKPCEEQYGSTYRCVSVLTVLHQDWLNGKEYKCKVSNKALPAPIEKT |
| | | | ISKAKGQPREPQVYTLPPSREEMTKNQVSLTCLVKGFYPSDIAVEWESNGQPENNYKT |
| | | | TPPVLDSDGSFFLYSKLTVDKSRWQQGNVFSCSVMHEALHNHYTQKSLSLSPGK |
| 2327 | DL 8-A7 CC | artificial | QVQLQESGPGLVKPSETLSLTCTVSGGSISSYYWSWIRQPPGKCLEWIGYVYYSGTTN |
| | x I2M x | | YNPSLKSRVTISVDTSKNQFSLKLSSVTAADTAVYYCASIAVTGFYFDYWGQGTLVTV |
| | scFc-Full | | SSGGGGSGGGGSGGGGSEIVLTQSPGTLSLSPGERVTLSCRASQRVNNNYLAWYQQRP |
| | Sequence | | GQAPRLLIYGASSRATGIPDRFSGSGSGTDFTLTISRLEPEDFAVYYCQQYDRSPLTF |
| | | | GCGTKLEIKSGGGGSEVQLVESGGGLVQPGGSLKLSCAASGFTFNKYAMNWVRQAPGK |
| | | | GMEWVARIRSKYNNYATYYADAVKDRFTISRDDSKNTLYLQMNNLKTEDTAVYYCVRA |
| | | | GNFGTSYISYWAYWGQGTLVTVSSGGGGSGGGGSGGGGSQTVVTQEPSLTVSPGGTVT |
| | | | ITCGSSTGAVTSGNYPNWVQKKPGQAPRGLIGGTKFLAPGTPARFSGSLSGGKAALTL |
| | | | SGVQPEDEAEYYCVLWYSNRWVFGSGTKLTVLGGGGDKTHTCPPCPAPELLGGPSVFL |
| | | | FPPKPKDTLMISRTPEVTCVVVDVSHEDPEVKFNWYVDGVEVHNAKTKPCEEQYGSTY |
| | | | RCVSVLTVLHQDWLNGKEYKCKVSNKALPAPIEKTISKAKGQPREPQVYTLPPSREEM |
| | | | TKNQVSLTCLVKGFYPSDIAVEWESNGQPENNYKTTPPVLDSDGSFFLYSKLTVDKSR |
| | | | WQQGNVFSCSVMHEALHNHYTQKSLSLSPGGGGSGGGGSGGGGSGGGGSGGGGSGGGG |
| | | | SDKTHTCPPCPAPELLGGPSVFLFPPKPKDTLMISRTPEVTCVVVDVSHEDPEVKENW |
| | | | YVDGVEVHNAKTKPCEEQYGSTYRCVSVLTVLHQDWLNGKEYKCKVSNKALPAPIEKT |
| | | | ISKAKGQPREPQVYTLPPSREEMTKNQVSLTCLVKGFYPSDIAVEWESNGQPENNYKT |
| | | | TPPVLDSDGSFFLYSKLTVDKSRWQQGNVFSCSVMHEALHNHYTQKSLSLSPGK |
| 2328 | DL 8-A7 CC | artificial | QVQLQESGPGLVKPSETLSLTCTVSGGSISSYYWSWIRQPPGKCLEWIGYVYYSGTTN |
| | x I2M2 x | | YNPSLKSRVTISVDTSKNQFSLKLSSVTAADTAVYYCASIAVTGFYFDYWGQGTLVTV |
| | scFc-Full | | SSGGGGSGGGGSGGGGSEIVLTQSPGTLSLSPGERVTLSCRASQRVNNNYLAWYQQRP |
| | Sequence | | GQAPRLLIYGASSRATGIPDRFSGSGSGTDFTLTISRLEPEDFAVYYCQQYDRSPLTF |
| | | | GCGTKLEIKSGGGGSEVQLVESGGGLVQPGGSLKLSCAASGFTENKYAINWVREAPGK |
| | | | GLEWVARIRSKYNNYATYYADAVKDRFTISRDDSKNTAYLQMNNLKTEDTAVYYCVRN |
| | | | ANFGTSYISYFAYWGQGTLVTVSSGGGGSGGGGSGGGGSQTVVTQEPSLTVSPGGTVT |
| | | | LTCGSSTGAVTSGNYPNWVQKKPGQAPRGLIGGTKFLAPGTPARFSGSLLGGKAALTL |
| | | | SGVQPEDEAEYYCVLWYSNRWVFGSGTKLTVLGGGGDKTHTCPPCPAPELLGGPSVFL |
| | | | FPPKPKDTLMISRTPEVTCVVVDVSHEDPEVKFNWYVDGVEVHNAKTKPCEEQYGSTY |
| | | | RCVSVLTVLHQDWLNGKEYKCKVSNKALPAPIEKTISKAKGQPREPQVYTLPPSREEM |
| | | | TKNQVSLTCLVKGFYPSDIAVEWESNGQPENNYKTTPPVLDSDGSFFLYSKLTVDKSR |
| | | | WQQGNVFSCSVMHEALHNHYTQKSLSLSPGGGGSGGGGSGGGGSGGGGSGGGGSGGGG |
| | | | SDKTHTCPPCPAPELLGGPSVFLFPPKPKDTLMISRTPEVTCVVVDVSHEDPEVKENW |
| | | | YVDGVEVHNAKTKPCEEQYGSTYRCVSVLTVLHQDWLNGKEYKCKVSNKALPAPIEKT |
| | | | ISKAKGQPREPQVYTLPPSREEMTKNQVSLTCLVKGFYPSDIAVEWESNGQPENNYKT |
| | | | TPPVLDSDGSFFLYSKLTVDKSRWQQGNVFSCSVMHEALHNHYTQKSLSLSPGK |
| 2329 | EGFRvIII CC | artificial | NYGMH |
| | -HCDR1 | | |
| 2330 | EGFRvIII CC | artificial | VIWYDGSDKYYADSVRG |
| | -HCDR2 | | |
| 2331 | EGFRvIII CC | artificial | DGYDILTGNPRDEDY |
| | -HCDR3 | | |
| 2332 | EGFRvIII CC | artificial | RSSQSLVHSDGNTYLS |
| | -LCDR1 | | |
| 2333 | EGFRvIII CC | artificial | RISRRFS |
| | -LCDR2 | | |
| 2334 | EGFRvIII CC | artificial | MQSTHVPRT |
| | -LCDR3 | | |
| 2335 | EGFRvIII CC | artificial | QVQLVESGGGVVQSGRSLRLSCAASGFTFRNYGMHWVRQAPGKCLEWVAVIWYDGSDK |
| | -VH | | YYADSVRGRFTISRDNSKNTLYLQMNSLRAEDTAVYYCARDGYDILTGNPRDFDYWGQ |
| | | | GTLVTVSS |
| 2336 | EGFRvIII CC | artificial | DTVMTQTPLSSHVTLGQPASISCRSSQSLVHSDGNTYLSWLQQRPGQPPRLLIYRISR |
| | -VL | | RFSGVPDRFSGSGAGTDFTLEISRVEAEDVGVYYCMQSTHVPRTFGCGTKVEIK |
| 2337 | EGFRvIII CC | artificial | QVQLVESGGGVVQSGRSLRLSCAASGFTFRNYGMHWVRQAPGKCLEWVAVIWYDGSDK |
| | x I2C- | | YYADSVRGRFTISRDNSKNTLYLQMNSLRAEDTAVYYCARDGYDILTGNPRDFDYWGQ |
| | Full | | GTLVTVSSGGGGSGGGGSGGGGSDTVMTQTPLSSHVTLGQPASISCRSSQSLVHSDGN |
| | Sequence | | TYLSWLQQRPGQPPRLLIYRISRRFSGVPDRFSGSGAGTDFTLEISRVEAEDVGVYYC |
| | | | MQSTHVPRTFGCGTKVEIKSGGGGSEVQLVESGGGLVQPGGSLKLSCAASGFTENKYA |
| | | | MNWVRQAPGKGLEWVARIRSKYNNYATYYADSVKDRFTISRDDSKNTAYLQMNNLKTE |
| | | | DTAVYYCVRHGNFGNSYISYWAYWGQGTLVTVSSGGGGSGGGGSGGGGSQTVVTQEPS |
| | | | LTVSPGGTVTLTCGSSTGAVTSGNYPNWVQQKPGQAPRGLIGGTKFLAPGTPARFSGS |
| | | | LLGGKAALTLSGVQPEDEAEYYCVLWYSNRWVFGGGTKLTVL |
| 2338 | EGFRvIII CC | artificial | QVQLVESGGGVVQSGRSLRLSCAASGFTFRNYGMHWVRQAPGKCLEWVAVIWYDGSDK |
| | x I2E- | | YYADSVRGRFTISRDNSKNTLYLQMNSLRAEDTAVYYCARDGYDILTGNPRDFDYWGQ |
| | Full | | GTLVTVSSGGGGSGGGGSGGGGSDTVMTQTPLSSHVTLGQPASISCRSSQSLVHSDGN |
| | Sequence | | TYLSWLQQRPGQPPRLLIYRISRRFSGVPDRFSGSGAGTDFTLEISRVEAEDVGVYYC |
| | | | MQSTHVPRTFGCGTKVEIKSGGGGSEVQLVESGGGLVQPGGSLKLSCAASGFTFNKYA |
| | | | INWVRQAPGKGLEWVARIRSKYNNYATYYADAVKDRFTISRDDSKNTVYLQMNNLKTE |
| | | | DTAVYYCARAGNFGSSYISYWAYWGQGTLVTVSSGGGGSGGGGSGGGGSQTVVTQEPS |
| | | | LTVSPGGTVTITCGSSTGAVTSGNYPNWVQKKPGQAPRGLIGGTKFLAPGTPARFSGS |
| | | | LSGGKAALTLSGVQPEDEAEYYCVLWYSNRWVFGSGTKLTVL |
| 2339 | EGFRvIII CC | artificial | QVQLVESGGGVVQSGRSLRLSCAASGFTFRNYGMHWVRQAPGKCLEWVAVIWYDGSDK |
| | x I2K- | | YYADSVRGRFTISRDNSKNTLYLQMNSLRAEDTAVYYCARDGYDILTGNPRDEDYWGQ |
| | Full | | GTLVTVSSGGGGSGGGGSGGGGSDTVMTQTPLSSHVTLGQPASISCRSSQSLVHSDGN |
| | Sequence | | TYLSWLQQRPGQPPRLLIYRISRRFSGVPDRFSGSGAGTDFTLEISRVEAEDVGVYYC |
| | | | MQSTHVPRTFGCGTKVEIKSGGGGSEVQLVESGGGLVQPGGSLKLSCAASGFTENKYA |
| | | | MNWVRQAPGKGLEWVARIRSKYNNYATYYAEAVKGRFTISRDDSKNTVYLQMNNLKTE |
| | | | DTAVYYCVRNENIGTSYISYWAYWGQGTLVTVSSGGGGSGGGGSGGGGSQTVVTQEPS |
| | | | LTVSPGGTVTITCGSSTGAVTSGNYPNWVQQKPGQAPRGLIGGTKFLAPGTPARFSGS |
| | | | LSGGKAALTLSGVQPEDEAEYYCVLWYSNRWVFGSGTKLTVL |
| 2340 | EGFRvIII CC | artificial | QVQLVESGGGVVQSGRSLRLSCAASGFTFRNYGMHWVRQAPGKCLEWVAVIWYDGSDK |
| | x I2L- | | YYADSVRGRFTISRDNSKNTLYLQMNSLRAEDTAVYYCARDGYDILTGNPRDFDYWGQ |
| | Full | | GTLVTVSSGGGGSGGGGSGGGGSDTVMTQTPLSSHVTLGQPASISCRSSQSLVHSDGN |
| | Sequence | | TYLSWLQQRPGQPPRLLIYRISRRFSGVPDRESGSGAGTDFTLEISRVEAEDVGVYYC |
| | | | MQSTHVPRTFGCGTKVEIKSGGGGSEVQLVESGGGLVQPGGSLKLSCAASGFTENKYA |
| | | | MNWVRQAPGKGMEWVARIRSKYNNYATYYADAVKDRFTISRDDSKNTLYLQMNNLKTE |
| | | | DTAVYYCVRAGNFGSSYISYFAYWGQGTLVTVSSGGGGSGGGGSGGGGSQTVVTQEPS |
| | | | LTVSPGGTVTITCGSSTGAVTSGNYPNWIQKKPGQAPRGLIGGTKFLAPGTPARFSGS |
| | | | LEGGKAALTLSGVQPEDEAEYYCVLYYSNRWVFGSGTKLTVL |
| 2341 | EGFRvIII CC | artificial | QVQLVESGGGVVQSGRSLRLSCAASGFTFRNYGMHWVRQAPGKCLEWVAVIWYDGSDK |
| | x I2M- | | YYADSVRGRFTISRDNSKNTLYLQMNSLRAEDTAVYYCARDGYDILTGNPRDEDYWGQ |
| | Full | | GTLVTVSSGGGGSGGGGSGGGGSDTVMTQTPLSSHVTLGQPASISCRSSQSLVHSDGN |
| | Sequence | | TYLSWLQQRPGQPPRLLIYRISRRFSGVPDRFSGSGAGTDFTLEISRVEAEDVGVYYC |
| | | | MQSTHVPRTFGCGTKVEIKSGGGGSEVQLVESGGGLVQPGGSLKLSCAASGFTENKYA |
| | | | MNWVRQAPGKGMEWVARIRSKYNNYATYYADAVKDRFTISRDDSKNTLYLQMNNLKTE |
| | | | DTAVYYCVRAGNFGTSYISYWAYWGQGTLVTVSSGGGGSGGGGSGGGGSQTVVTQEPS |
| | | | LTVSPGGTVTITCGSSTGAVTSGNYPNWVQKKPGQAPRGLIGGTKFLAPGTPARFSGS |
| | | | LSGGKAALTLSGVQPEDEAEYYCVLWYSNRWVFGSGTKLTVL |
| 2342 | EGFRvIII CC | artificial | QVQLVESGGGVVQSGRSLRLSCAASGFTFRNYGMHWVRQAPGKCLEWVAVIWYDGSDK |
| | x I2M2- | | YYADSVRGRFTISRDNSKNTLYLQMNSLRAEDTAVYYCARDGYDILTGNPRDEDYWGQ |
| | Full | | GTLVTVSSGGGGSGGGGSGGGGSDTVMTQTPLSSHVTLGQPASISCRSSQSLVHSDGN |
| | Sequence | | TYLSWLQQRPGQPPRLLIYRISRRFSGVPDRFSGSGAGTDFTLEISRVEAEDVGVYYC |
| | | | MQSTHVPRTFGCGTKVEIKSGGGGSEVQLVESGGGLVQPGGSLKLSCAASGFTENKYA |
| | | | INWVREAPGKGLEWVARIRSKYNNYATYYADAVKDRFTISRDDSKNTAYLQMNNLKTE |
| | | | DTAVYYCVRNANFGTSYISYFAYWGQGTLVTVSSGGGGSGGGGSGGGGSQTVVTQEPS |
| | | | LTVSPGGTVTLTCGSSTGAVTSGNYPNWVQKKPGQAPRGLIGGTKFLAPGTPARFSGS |
| | | | LLGGKAALTLSGVQPEDEAEYYCVLWYSNRWVFGSGTKLTVL |
| 2343 | FL 7-A8 CC | artificial | NARMGVS |
| | -HCDR1 | | |
| 2344 | FL 7-A8 CC | artificial | HIFSNDEKSYSTSLKN |
| | -HCDR2 | | |
| 2345 | FL 7-A8 CC | artificial | IVGYGSGWYGFFDY |
| | -HCDR3 | | |
| 2346 | FL 7-A8 CC | artificial | RASQGIRNDLG |
| | -LCDR1 | | |
| 2347 | FL 7-A8 CC | artificial | AASTLQS |
| | -LCDR2 | | |
| 2348 | FL 7-A8 CC | artificial | LQHNSYPLT |
| | -LCDR3 | | |
| 2349 | FL 7-A8 CC | artificial | QVTLKESGPTLVKPTETLTLTCTLSGFSLNNARMGVSWIRQPPGKCLEWLAHIFSNDE |
| | -VH | | KSYSTSLKNRLTISKDSSKTQVVLTMTNVDPVDTATYYCARIVGYGSGWYGFFDYWGQ |
| | | | GTLVTVSS |
| 2350 | FL 7-A8 CC | artificial | DIQMTQSPSSLSASVGDRVTITCRASQGIRNDLGWYQQKPGKAPKRLIYAASTLQSGV |
| | -VL | | PSRFSGSGSGTEFTLTISSLQPEDFATYYCLQHNSYPLTFGCGTKVEIK |
| 2351 | FL 7-A8 CC | artificial | EIQMTQSPSSLSASVGDRVTITCRASQGIRNDLGWYQQKPGKAPKRLIYAASTLQSGV |
| | EI GQ-VL | | PSRFSGSGSGTEFTLTISSLQPEDFATYYCLQHNSYPLTFGCGTKVEIK |
| 2352 | FL 7-A8 CC | artificial | QVTLKESGPTLVKPTETLTLTCTLSGFSLNNARMGVSWIRQPPGKCLEWLAHIFSNDE |
| | x I2Cx scFc | | KSYSTSLKNRLTISKDSSKTQVVLTMTNVDPVDTATYYCARIVGYGSGWYGFFDYWGQ |
| | -Full | | GTLVTVSSGGGGSGGGGSGGGGSDIQMTQSPSSLSASVGDRVTITCRASQGIRNDLGW |
| | Sequence | | YQQKPGKAPKRLIYAASTLQSGVPSRFSGSGSGTEFTLTISSLQPEDFATYYCLQHNS |
| | | | YPLTFGCGTKVEIKSGGGGSEVQLVESGGGLVQPGGSLKLSCAASGFTENKYAMNWVR |
| | | | QAPGKGLEWVARIRSKYNNYATYYADSVKDRFTISRDDSKNTAYLQMNNLKTEDTAVY |
| | | | YCVRHGNFGNSYISYWAYWGQGTLVTVSSGGGGSGGGGSGGGGSQTVVTQEPSLTVSP |
| | | | GGTVTLTCGSSTGAVTSGNYPNWVQQKPGQAPRGLIGGTKFLAPGTPARFSGSLLGGK |
| | | | AALTLSGVQPEDEAEYYCVLWYSNRWVFGGGTKLTVLGGGGDKTHTCPPCPAPELLGG |
| | | | PSVFLFPPKPKDTLMISRTPEVTCVVVDVSHEDPEVKFNWYVDGVEVHNAKTKPCEEQ |
| | | | YGSTYRCVSVLTVLHQDWLNGKEYKCKVSNKALPAPIEKTISKAKGQPREPQVYTLPP |
| | | | SREEMTKNQVSLTCLVKGFYPSDIAVEWESNGQPENNYKTTPPVLDSDGSFFLYSKLT |
| | | | VDKSRWQQGNVFSCSVMHEALHNHYTQKSLSLSPGKGGGGSGGGGSGGGGSGGGGSGG |
| | | | GGSGGGGSDKTHTCPPCPAPELLGGPSVFLFPPKPKDTLMISRTPEVTCVVVDVSHED |
| | | | PEVKFNWYVDGVEVHNAKTKPCEEQYGSTYRCVSVLTVLHQDWLNGKEYKCKVSNKAL |
| | | | PAPIEKTISKAKGQPREPQVYTLPPSREEMTKNQVSLTCLVKGFYPSDIAVEWESNGQ |
| | | | PENNYKTTPPVLDSDGSFFLYSKLTVDKSRWQQGNVFSCSVMHEALHNHYTQKSLSLS |
| | | | PGK |
| 2353 | FL 7-A8 CC | artificial | QVTLKESGPTLVKPTETLTLTCTLSGFSLNNARMGVSWIRQPPGKCLEWLAHIFSNDE |
| | x I2Ex scFc | | KSYSTSLKNRLTISKDSSKTQVVLTMTNVDPVDTATYYCARIVGYGSGWYGFFDYWGQ |
| | -Full | | GTLVTVSSGGGGSGGGGSGGGGSDIQMTQSPSSLSASVGDRVTITCRASQGIRNDLGW |
| | Sequence | | YQQKPGKAPKRLIYAASTLQSGVPSRFSGSGSGTEFTLTISSLQPEDFATYYCLQHNS |
| | | | YPLTFGCGTKVEIKSGGGGSEVQLVESGGGLVQPGGSLKLSCAASGFTENKYAINWVR |
| | | | QAPGKGLEWVARIRSKYNNYATYYADAVKDRFTISRDDSKNTVYLQMNNLKTEDTAVY |
| | | | YCARAGNFGSSYISYWAYWGQGTLVTVSSGGGGSGGGGSGGGGSQTVVTQEPSLTVSP |
| | | | GGTVTITCGSSTGAVTSGNYPNWVQKKPGQAPRGLIGGTKFLAPGTPARFSGSLSGGK |
| | | | AALTLSGVQPEDEAEYYCVLWYSNRWVFGSGTKLTVLGGGGDKTHTCPPCPAPELLGG |
| | | | PSVFLFPPKPKDTLMISRTPEVTCVVVDVSHEDPEVKFNWYVDGVEVHNAKTKPCEEQ |
| | | | YGSTYRCVSVLTVLHQDWLNGKEYKCKVSNKALPAPIEKTISKAKGQPREPQVYTLPP |
| | | | SREEMTKNQVSLTCLVKGFYPSDIAVEWESNGQPENNYKTTPPVLDSDGSFFLYSKLT |
| | | | VDKSRWQQGNVFSCSVMHEALHNHYTQKSLSLSPGKGGGGSGGGGSGGGGSGGGGSGG |
| | | | GGSGGGGSDKTHTCPPCPAPELLGGPSVFLFPPKPKDTLMISRTPEVTCVVVDVSHED |
| | | | PEVKFNWYVDGVEVHNAKTKPCEEQYGSTYRCVSVLTVLHQDWLNGKEYKCKVSNKAL |
| | | | PAPIEKTISKAKGQPREPQVYTLPPSREEMTKNQVSLTCLVKGFYPSDIAVEWESNGQ |
| | | | PENNYKTTPPVLDSDGSFFLYSKLTVDKSRWQQGNVFSCSVMHEALHNHYTQKSLSLS |
| | | | PGK |
| 2354 | FL 7-A8 CC | artificial | QVTLKESGPTLVKPTETLTLTCTLSGFSLNNARMGVSWIRQPPGKCLEWLAHIFSNDE |
| | x I2Kx scFc | | KSYSTSLKNRLTISKDSSKTQVVLTMTNVDPVDTATYYCARIVGYGSGWYGFFDYWGQ |
| | -Full | | GTLVTVSSGGGGSGGGGSGGGGSDIQMTQSPSSLSASVGDRVTITCRASQGIRNDLGW |
| | Sequence | | YQQKPGKAPKRLIYAASTLQSGVPSRFSGSGSGTEFTLTISSLQPEDFATYYCLQHNS |
| | | | YPLTFGCGTKVEIKSGGGGSEVQLVESGGGLVQPGGSLKLSCAASGFTENKYAMNWVR |
| | | | QAPGKGLEWVARIRSKYNNYATYYAEAVKGRFTISRDDSKNTVYLQMNNLKTEDTAVY |
| | | | YCVRNENIGTSYISYWAYWGQGTLVTVSSGGGGSGGGGSGGGGSQTVVTQEPSLTVSP |
| | | | GGTVTITCGSSTGAVTSGNYPNWVQQKPGQAPRGLIGGTKFLAPGTPARFSGSLSGGK |
| | | | AALTLSGVQPEDEAEYYCVLWYSNRWVFGSGTKLTVLGGGGDKTHTCPPCPAPELLGG |
| | | | PSVFLFPPKPKDTLMISRTPEVTCVVVDVSHEDPEVKENWYVDGVEVHNAKTKPCEEQ |
| | | | YGSTYRCVSVLTVLHQDWLNGKEYKCKVSNKALPAPIEKTISKAKGQPREPQVYTLPP |
| | | | SREEMTKNQVSLTCLVKGFYPSDIAVEWESNGQPENNYKTTPPVLDSDGSFFLYSKLT |
| | | | VDKSRWQQGNVFSCSVMHEALHNHYTQKSLSLSPGKGGGGSGGGGSGGGGSGGGGSGG |
| | | | GGSGGGGSDKTHTCPPCPAPELLGGPSVFLFPPKPKDTLMISRTPEVTCVVVDVSHED |
| | | | PEVKFNWYVDGVEVHNAKTKPCEEQYGSTYRCVSVLTVLHQDWLNGKEYKCKVSNKAL |
| | | | PAPIEKTISKAKGQPREPQVYTLPPSREEMTKNQVSLTCLVKGFYPSDIAVEWESNGQ |
| | | | PENNYKTTPPVLDSDGSFFLYSKLTVDKSRWQQGNVFSCSVMHEALHNHYTQKSLSLS |
| | | | PGK |
| 2355 | FL 7-A8 CC | artificial | QVTLKESGPTLVKPTETLTLTCTLSGFSLNNARMGVSWIRQPPGKCLEWLAHIFSNDE |
| | x I2Lx scFc | | KSYSTSLKNRLTISKDSSKTQVVLTMTNVDPVDTATYYCARIVGYGSGWYGFFDYWGQ |
| | -Full | | GTLVTVSSGGGGSGGGGSGGGGSDIQMTQSPSSLSASVGDRVTITCRASQGIRNDLGW |
| | Sequence | | YQQKPGKAPKRLIYAASTLQSGVPSRFSGSGSGTEFTLTISSLQPEDFATYYCLQHNS |
| | | | YPLTFGCGTKVEIKSGGGGSEVQLVESGGGLVQPGGSLKLSCAASGFTENKYAMNWVR |
| | | | QAPGKGMEWVARIRSKYNNYATYYADAVKDRFTISRDDSKNTLYLQMNNLKTEDTAVY |
| | | | YCVRAGNFGSSYISYFAYWGQGTLVTVSSGGGGSGGGGSGGGGSQTVVTQEPSLTVSP |
| | | | GGTVTITCGSSTGAVTSGNYPNWIQKKPGQAPRGLIGGTKFLAPGTPARFSGSLEGGK |
| | | | AALTLSGVQPEDEAEYYCVLYYSNRWVFGSGTKLTVLGGGGDKTHTCPPCPAPELLGG |
| | | | PSVFLFPPKPKDTLMISRTPEVTCVVVDVSHEDPEVKENWYVDGVEVHNAKTKPCEEQ |
| | | | YGSTYRCVSVLTVLHQDWLNGKEYKCKVSNKALPAPIEKTISKAKGQPREPQVYTLPP |
| | | | SREEMTKNQVSLTCLVKGFYPSDIAVEWESNGQPENNYKTTPPVLDSDGSFFLYSKLT |
| | | | VDKSRWQQGNVFSCSVMHEALHNHYTQKSLSLSPGKGGGGSGGGGSGGGGSGGGGSGG |
| | | | GGSGGGGSDKTHTCPPCPAPELLGGPSVFLFPPKPKDTLMISRTPEVTCVVVDVSHED |
| | | | PEVKFNWYVDGVEVHNAKTKPCEEQYGSTYRCVSVLTVLHQDWLNGKEYKCKVSNKAL |
| | | | PAPIEKTISKAKGQPREPQVYTLPPSREEMTKNQVSLTCLVKGFYPSDIAVEWESNGQ |
| | | | PENNYKTTPPVLDSDGSFFLYSKLTVDKSRWQQGNVFSCSVMHEALHNHYTQKSLSLS |
| | | | PGK |
| 2356 | FL 7-A8 CC | artificial | QVTLKESGPTLVKPTETLTLTCTLSGFSLNNARMGVSWIRQPPGKCLEWLAHIFSNDE |
| | x I2M2x | | KSYSTSLKNRLTISKDSSKTQVVLTMTNVDPVDTATYYCARIVGYGSGWYGFFDYWGQ |
| | ScFc-Full | | GTLVTVSSGGGGSGGGGSGGGGSDIQMTQSPSSLSASVGDRVTITCRASQGIRNDLGW |
| | Sequence | | YQQKPGKAPKRLIYAASTLQSGVPSRFSGSGSGTEFTLTISSLQPEDFATYYCLQHNS |
| | | | YPLTFGCGTKVEIKSGGGGSEVQLVESGGGLVQPGGSLKLSCAASGFTENKYAINWVR |
| | | | EAPGKGLEWVARIRSKYNNYATYYADAVKDRFTISRDDSKNTAYLQMNNLKTEDTAVY |
| | | | YCVRNANFGTSYISYFAYWGQGTLVTVSSGGGGSGGGGSGGGGSQTVVTQEPSLTVSP |
| | | | GGTVTLTCGSSTGAVTSGNYPNWVQKKPGQAPRGLIGGTKFLAPGTPARFSGSLLGGK |
| | | | AALTLSGVQPEDEAEYYCVLWYSNRWVFGSGTKLTVLGGGGDKTHTCPPCPAPELLGG |
| | | | PSVFLFPPKPKDTLMISRTPEVTCVVVDVSHEDPEVKENWYVDGVEVHNAKTKPCEEQ |
| | | | YGSTYRCVSVLTVLHQDWLNGKEYKCKVSNKALPAPIEKTISKAKGQPREPQVYTLPP |
| | | | SREEMTKNQVSLTCLVKGFYPSDIAVEWESNGQPENNYKTTPPVLDSDGSFFLYSKLT |
| | | | VDKSRWQQGNVFSCSVMHEALHNHYTQKSLSLSPGKGGGGSSGGGGGGGGSGGGGSGG |
| | | | GGSGGGGSDKTHTCPPCPAPELLGGPSVFLFPPKPKDTLMISRTPEVTCVVVDVSHED |
| | | | PEVKFNWYVDGVEVHNAKTKPCEEQYGSTYRCVSVLTVLHQDWLNGKEYKCKVSNKAL |
| | | | PAPIEKTISKAKGQPREPQVYTLPPSREEMTKNQVSLTCLVKGFYPSDIAVEWESNGQ |
| | | | PENNYKTTPPVLDSDGSFFLYSKLTVDKSRWQQGNVFSCSVMHEALHNHYTQKSLSLS |
| | | | PGK |
| 2357 | FL 7-A8 CC | artificial | QVTLKESGPTLVKPTETLTLTCTLSGFSLNNARMGVSWIRQPPGKCLEWLAHIFSNDE |
| | x I2Mx scFc | | KSYSTSLKNRLTISKDSSKTQVVLTMTNVDPVDTATYYCARIVGYGSGWYGFFDYWGQ |
| | -Full | | GTLVTVSSGGGGSGGGGSGGGGSDIQMTQSPSSLSASVGDRVTITCRASQGIRNDLGW |
| | Sequence | | YQQKPGKAPKRLIYAASTLQSGVPSRFSGSGSGTEFTLTISSLQPEDFATYYCLQHNS |
| | | | YPLTFGCGTKVEIKSGGGGSEVQLVESGGGLVQPGGSLKLSCAASGFTENKYAMNWVR |
| | | | QAPGKGMEWVARIRSKYNNYATYYADAVKDRFTISRDDSKNTLYLQMNNLKTEDTAVY |
| | | | YCVRAGNFGTSYISYWAYWGQGTLVTVSSGGGGSGGGGSGGGGSQTVVTQEPSLTVSP |
| | | | GGTVTITCGSSTGAVTSGNYPNWVQKKPGQAPRGLIGGTKFLAPGTPARFSGSLSGGK |
| | | | AALTLSGVQPEDEAEYYCVLWYSNRWVFGSGTKLTVLGGGGDKTHTCPPCPAPELLGG |
| | | | PSVFLFPPKPKDTLMISRTPEVTCVVVDVSHEDPEVKENWYVDGVEVHNAKTKPCEEQ |
| | | | YGSTYRCVSVLTVLHQDWLNGKEYKCKVSNKALPAPIEKTISKAKGQPREPQVYTLPP |
| | | | SREEMTKNQVSLTCLVKGFYPSDIAVEWESNGQPENNYKTTPPVLDSDGSFFLYSKLT |
| | | | VDKSRWQQGNVFSCSVMHEALHNHYTQKSLSLSPGKGGGGSGGGGSGGGGSGGGGSGG |
| | | | GGSGGGGSDKTHTCPPCPAPELLGGPSVFLFPPKPKDTLMISRTPEVTCVVVDVSHED |
| | | | PEVKFNWYVDGVEVHNAKTKPCEEQYGSTYRCVSVLTVLHQDWLNGKEYKCKVSNKAL |
| | | | PAPIEKTISKAKGQPREPQVYTLPPSREEMTKNQVSLTCLVKGFYPSDIAVEWESNGQ |
| | | | PENNYKTTPPVLDSDGSFFLYSKLTVDKSRWQQGNVFSCSVMHEALHNHYTQKSLSLS |
| | | | PGK |
| 2358 | MA 10-B5 CC | artificial | NAWMS |
| | -HCDR1 | | |
| 2359 | MA 10-B5 CC | artificial | RIRSRSYGGTTDYAAPVKG |
| | -HCDR2 | | |
| 2360 | MA 10-B5 CC | artificial | PSYSGSYYNYFSVMDV |
| | HCDR3 | | |
| 2361 | MA 10-B5 CC | artificial | RTSQSISSYLN |
| | LCDR1 | | |
| 2362 | MA 10-B5 CC | artificial | AASSLQG |
| | -LCDR2 | | |
| 2363 | MA 10-B5 CC | artificial | QQTYSMPFT |
| | -LCDR3 | | |
| 2364 | MA 10-B5 CC | artificial | EVQLVESGGGLVQPGGSLRLSCAASGFTFSNAWMSWVRQAPGKCLEWVGRIRSRSYGG |
| | -VH | | TTDYAAPVKGRFTISRDDSKNTLFLQMNSLKTEDTAVYYCTTPSYSGSYYNYFSVMDV |
| | | | WGQGTTVTVSS |
| 2365 | MA 10-B5 CC | artificial | DIQMTQSPSSLSASVGDRVTITCRTSQSISSYLNWYQQKPGRAPKLLIFAASSLQGGV |
| | -VL | | PSRFSGSGSGTDFTLTISSLQPEDFATYYCQQTYSMPFTFGCGTKVEIK |
| 2366 | MA 10-B5 CC | artificial | EIQMTQSPSSLSASVGDRVTITCRTSQSISSYLNWYQQKPGRAPKLLIFAASSLQGGV |
| | EI GQ-VL | | PSRFSGSGSGTDFTLTISSLQPEDFATYYCQQTYSMPFTFGCGTKVEIK |
| 2367 | MA 10-B5 CC | artificial | EVQLVESGGGLVQPGGSLRLSCAASGFTFSNAWMSWVRQAPGKCLEWVGRIRSRSYGG |
| | x I2C x | | TTDYAAPVKGRFTISRDDSKNTLFLQMNSLKTEDTAVYYCTTPSYSGSYYNYFSVMDV |
| | scFc-Full | | WGQGTTVTVSSGGGGSGGGGSGGGGSDIQMTQSPSSLSASVGDRVTITCRTSQSISSY |
| | Sequence | | LNWYQQKPGRAPKLLIFAASSLQGGVPSRFSGSGSGTDFTLTISSLQPEDFATYYCQQ |
| | | | TYSMPFTFGCGTKVEIKSGGGGSEVQLVESGGGLVQPGGSLKLSCAASGFTFNKYAMN |
| | | | WVRQAPGKGLEWVARIRSKYNNYATYYADSVKDRFTISRDDSKNTAYLQMNNLKTEDT |
| | | | AVYYCVRHGNFGNSYISYWAYWGQGTLVTVSSGGGGSGGGGSGGGGSQTVVTQEPSLT |
| | | | VSPGGTVTLTCGSSTGAVTSGNYPNWVQQKPGQAPRGLIGGTKFLAPGTPARFSGSLL |
| | | | GGKAALTLSGVQPEDEAEYYCVLWYSNRWVFGGGTKLTVLGGGGDKTHTCPPCPAPEL |
| | | | LGGPSVFLFPPKPKDTLMISRTPEVTCVVVDVSHEDPEVKENWYVDGVEVHNAKTKPC |
| | | | EEQYGSTYRCVSVLTVLHQDWLNGKEYKCKVSNKALPAPIEKTISKAKGQPREPQVYT |
| | | | LPPSREEMTKNQVSLTCLVKGFYPSDIAVEWESNGQPENNYKTTPPVLDSDGSFFLYS |
| | | | KLTVDKSRWQQGNVFSCSVMHEALHNHYTQKSLSLSPGKGGGGSGGGGSGGGGSGGGG |
| | | | SGGGGSGGGGSDKTHTCPPCPAPELLGGPSVFLFPPKPKDTLMISRTPEVTCVVVDVS |
| | | | HEDPEVKFNWYVDGVEVHNAKTKPCEEQYGSTYRCVSVLTVLHQDWLNGKEYKCKVSN |
| | | | KALPAPIEKTISKAKGQPREPQVYTLPPSREEMTKNQVSLTCLVKGFYPSDIAVEWES |
| | | | NGQPENNYKTTPPVLDSDGSFFLYSKLTVDKSRWQQGNVFSCSVMHEALHNHYTQKSL |
| | | | SLSPGK |
| 2368 | MA 10-B5 CC | artificial | EVQLVESGGGLVQPGGSLRLSCAASGFTFSNAWMSWVRQAPGKCLEWVGRIRSRSYGG |
| | x I2E x | | TTDYAAPVKGRFTISRDDSKNTLFLQMNSLKTEDTAVYYCTTPSYSGSYYNYFSVMDV |
| | scFc-Full | | WGQGTTVTVSSGGGGSGGGGSGGGGSDIQMTQSPSSLSASVGDRVTITCRTSQSISSY |
| | Sequence | | LNWYQQKPGRAPKLLIFAASSLQGGVPSRFSGSGSGTDFTLTISSLQPEDFATYYCQQ |
| | | | TYSMPFTFGCGTKVEIKSGGGGSEVQLVESGGGLVQPGGSLKLSCAASGFTENKYAIN |
| | | | WVRQAPGKGLEWVARIRSKYNNYATYYADAVKDRFTISRDDSKNTVYLQMNNLKTEDT |
| | | | AVYYCARAGNFGSSYISYWAYWGQGTLVTVSSGGGGSGGGGSGGGGSQTVVTQEPSLT |
| | | | VSPGGTVTITCGSSTGAVTSGNYPNWVQKKPGQAPRGLIGGTKFLAPGTPARFSGSLS |
| | | | GGKAALTLSGVQPEDEAEYYCVLWYSNRWVFGSGTKLTVLGGGGDKTHTCPPCPAPEL |
| | | | LGGPSVFLFPPKPKDTLMISRTPEVTCVVVDVSHEDPEVKENWYVDGVEVHNAKTKPC |
| | | | EEQYGSTYRCVSVLTVLHQDWLNGKEYKCKVSNKALPAPIEKTISKAKGQPREPQVYT |
| | | | LPPSREEMTKNQVSLTCLVKGFYPSDIAVEWESNGQPENNYKTTPPVLDSDGSFFLYS |
| | | | KLTVDKSRWQQGNVFSCSVMHEALHNHYTQKSLSLSPGKGGGGSGGGGSGGGGSGGGG |
| | | | SGGGGSGGGGSDKTHTCPPCPAPELLGGPSVFLFPPKPKDTLMISRTPEVTCVVVDVS |
| | | | HEDPEVKFNWYVDGVEVHNAKTKPCEEQYGSTYRCVSVLTVLHQDWLNGKEYKCKVSN |
| | | | KALPAPIEKTISKAKGQPREPQVYTLPPSREEMTKNQVSLTCLVKGFYPSDIAVEWES |
| | | | NGQPENNYKTTPPVLDSDGSFFLYSKLTVDKSRWQQGNVFSCSVMHEALHNHYTQKSL |
| | | | SLSPGK |
| 2369 | MA 10-B5 CC | artificial | EVQLVESGGGLVQPGGSLRLSCAASGFTFSNAWMSWVRQAPGKCLEWVGRIRSRSYGG |
| | x I2K x | | TTDYAAPVKGRFTISRDDSKNTLFLQMNSLKTEDTAVYYCTTPSYSGSYYNYFSVMDV |
| | scFc-Full | | WGQGTTVTVSSGGGGSGGGGSGGGGSDIQMTQSPSSLSASVGDRVTITCRTSQSISSY |
| | Sequence | | LNWYQQKPGRAPKLLIFAASSLQGGVPSRFSGSGSGTDFTLTISSLQPEDFATYYCQQ |
| | | | TYSMPFTFGCGTKVEIKSGGGGSEVQLVESGGGLVQPGGSLKLSCAASGFTENKYAMN |
| | | | WVRQAPGKGLEWVARIRSKYNNYATYYAEAVKGRFTISRDDSKNTVYLQMNNLKTEDT |
| | | | AVYYCVRNENIGTSYISYWAYWGQGTLVTVSSGGGGSGGGGSGGGGSQTVVTQEPSLT |
| | | | VSPGGTVTITCGSSTGAVTSGNYPNWVQQKPGQAPRGLIGGTKFLAPGTPARFSGSLS |
| | | | GGKAALTLSGVQPEDEAEYYCVLWYSNRWVFGSGTKLTVLGGGGDKTHTCPPCPAPEL |
| | | | LGGPSVFLFPPKPKDTLMISRTPEVTCVVVDVSHEDPEVKFNWYVDGVEVHNAKTKPC |
| | | | EEQYGSTYRCVSVLTVLHQDWLNGKEYKCKVSNKALPAPIEKTISKAKGQPREPQVYT |
| | | | LPPSREEMTKNQVSLTCLVKGFYPSDIAVEWESNGQPENNYKTTPPVLDSDGSFFLYS |
| | | | KLTVDKSRWQQGNVFSCSVMHEALHNHYTQKSLSLSPGKGGGGSGGGGSGGGGSGGGG |
| | | | SGGGGSGGGGSDKTHTCPPCPAPELLGGPSVFLFPPKPKDTLMISRTPEVTCVVVDVS |
| | | | HEDPEVKFNWYVDGVEVHNAKTKPCEEQYGSTYRCVSVLTVLHQDWLNGKEYKCKVSN |
| | | | KALPAPIEKTISKAKGQPREPQVYTLPPSREEMTKNQVSLTCLVKGFYPSDIAVEWES |
| | | | NGQPENNYKTTPPVLDSDGSFFLYSKLTVDKSRWQQGNVFSCSVMHEALHNHYTQKSL |
| | | | SLSPGK |
| 2370 | MA 10-B5 CC | artificial | EVQLVESGGGLVQPGGSLRLSCAASGFTFSNAWMSWVRQAPGKCLEWVGRIRSRSYGG |
| | x I2L x | | TTDYAAPVKGRFTISRDDSKNTLFLQMNSLKTEDTAVYYCTTPSYSGSYYNYFSVMDV |
| | scFc-Full | | WGQGTTVTVSSGGGGSGGGGSGGGGSDIQMTQSPSSLSASVGDRVTITCRTSQSISSY |
| | Sequence | | LNWYQQKPGRAPKLLIFAASSLQGGVPSRFSGSGSGTDFTLTISSLQPEDFATYYCQQ |
| | | | TYSMPFTFGCGTKVEIKSGGGGSEVQLVESGGGLVQPGGSLKLSCAASGFTENKYAMN |
| | | | WVRQAPGKGMEWVARIRSKYNNYATYYADAVKDRFTISRDDSKNTLYLQMNNLKTEDT |
| | | | AVYYCVRAGNFGSSYISYFAYWGQGTLVTVSSGGGGSGGGGSGGGGSQTVVTQEPSLT |
| | | | VSPGGTVTITCGSSTGAVTSGNYPNWIQKKPGQAPRGLIGGTKFLAPGTPARFSGSLE |
| | | | GGKAALTLSGVQPEDEAEYYCVLYYSNRWVFGSGTKLTVLGGGGDKTHTCPPCPAPEL |
| | | | LGGPSVFLFPPKPKDTLMISRTPEVTCVVVDVSHEDPEVKFNWYVDGVEVHNAKTKPC |
| | | | EEQYGSTYRCVSVLTVLHQDWLNGKEYKCKVSNKALPAPIEKTISKAKGQPREPQVYT |
| | | | LPPSREEMTKNQVSLTCLVKGFYPSDIAVEWESNGQPENNYKTTPPVLDSDGSFFLYS |
| | | | KLTVDKSRWQQGNVFSCSVMHEALHNHYTQKSLSLSPGKGGGGSGGGGSGGGGSGGGG |
| | | | SGGGGSGGGGSDKTHTCPPCPAPELLGGPSVFLFPPKPKDTLMISRTPEVTCVVVDVS |
| | | | HEDPEVKFNWYVDGVEVHNAKTKPCEEQYGSTYRCVSVLTVLHQDWLNGKEYKCKVSN |
| | | | KALPAPIEKTISKAKGQPREPQVYTLPPSREEMTKNQVSLTCLVKGFYPSDIAVEWES |
| | | | NGQPENNYKTTPPVLDSDGSFFLYSKLTVDKSRWQQGNVFSCSVMHEALHNHYTQKSL |
| | | | SLSPGK |
| 2371 | MA 10-B5 CC | artificial | EVQLVESGGGLVQPGGSLRLSCAASGFTFSNAWMSWVRQAPGKCLEWVGRIRSRSYGG |
| | x I2M x | | TTDYAAPVKGRFTISRDDSKNTLFLQMNSLKTEDTAVYYCTTPSYSGSYYNYFSVMDV |
| | scFc-Full | | WGQGTTVTVSSGGGGSGGGGSGGGGSDIQMTQSPSSLSASVGDRVTITCRTSQSISSY |
| | Sequence | | LNWYQQKPGRAPKLLIFAASSLQGGVPSRFSGSGSGTDFTLTISSLQPEDFATYYCQQ |
| | | | TYSMPFTFGCGTKVEIKSGGGGSEVQLVESGGGLVQPGGSLKLSCAASGFTENKYAMN |
| | | | WVRQAPGKGMEWVARIRSKYNNYATYYADAVKDRFTISRDDSKNTLYLQMNNLKTEDT |
| | | | AVYYCVRAGNFGTSYISYWAYWGQGTLVTVSSGGGGSGGGGSGGGGSQTVVTQEPSLT |
| | | | VSPGGTVTITCGSSTGAVTSGNYPNWVQKKPGQAPRGLIGGTKFLAPGTPARFSGSLS |
| | | | GGKAALTLSGVQPEDEAEYYCVLWYSNRWVFGSGTKLTVLGGGGDKTHTCPPCPAPEL |
| | | | LGGPSVFLFPPKPKDTLMISRTPEVTCVVVDVSHEDPEVKENWYVDGVEVHNAKTKPC |
| | | | EEQYGSTYRCVSVLTVLHQDWLNGKEYKCKVSNKALPAPIEKTISKAKGQPREPQVYT |
| | | | LPPSREEMTKNQVSLTCLVKGFYPSDIAVEWESNGQPENNYKTTPPVLDSDGSFFLYS |
| | | | KLTVDKSRWQQGNVFSCSVMHEALHNHYTQKSLSLSPGKGGGGSGGGGSGGGGSGGGG |
| | | | SGGGGSGGGGSDKTHTCPPCPAPELLGGPSVFLFPPKPKDTLMISRTPEVTCVVVDVS |
| | | | HEDPEVKFNWYVDGVEVHNAKTKPCEEQYGSTYRCVSVLTVLHQDWLNGKEYKCKVSN |
| | | | KALPAPIEKTISKAKGQPREPQVYTLPPSREEMTKNQVSLTCLVKGFYPSDIAVEWES |
| | | | NGQPENNYKTTPPVLDSDGSFFLYSKLTVDKSRWQQGNVFSCSVMHEALHNHYTQKSL |
| | | | SLSPGK |
| 2372 | MA 10-B5 CC | artificial | EVQLVESGGGLVQPGGSLRLSCAASGFTFSNAWMSWVRQAPGKCLEWVGRIRSRSYGG |
| | x I2M2 x | | TTDYAAPVKGRFTISRDDSKNTLFLQMNSLKTEDTAVYYCTTPSYSGSYYNYFSVMDV |
| | ScFc-Full | | WGQGTTVTVSSGGGGSGGGGSGGGGSDIQMTQSPSSLSASVGDRVTITCRTSQSISSY |
| | Sequence | | LNWYQQKPGRAPKLLIFAASSLQGGVPSRFSGSGSGTDFTLTISSLQPEDFATYYCQQ |
| | | | TYSMPFTFGCGTKVEIKSGGGGSEVQLVESGGGLVQPGGSLKLSCAASGFTFNKYAIN |
| | | | WVREAPGKGLEWVARIRSKYNNYATYYADAVKDRFTISRDDSKNTAYLQMNNLKTEDT |
| | | | AVYYCVRNANFGTSYISYFAYWGQGTLVTVSSGGGGSGGGGSGGGGSQTVVTQEPSLT |
| | | | VSPGGTVTLTCGSSTGAVTSGNYPNWVQKKPGQAPRGLIGGTKFLAPGTPARFSGSLL |
| | | | GGKAALTLSGVQPEDEAEYYCVLWYSNRWVFGSGTKLTVLGGGGDKTHTCPPCPAPEL |
| | | | LGGPSVFLFPPKPKDTLMISRTPEVTCVVVDVSHEDPEVKENWYVDGVEVHNAKTKPC |
| | | | EEQYGSTYRCVSVLTVLHQDWLNGKEYKCKVSNKALPAPIEKTISKAKGQPREPQVYT |
| | | | LPPSREEMTKNQVSLTCLVKGFYPSDIAVEWESNGQPENNYKTTPPVLDSDGSFFLYS |
| | | | KLTVDKSRWQQGNVFSCSVMHEALHNHYTQKSLSLSPGKGGGGSGGGGSGGGGSGGGG |
| | | | SGGGGSGGGGSDKTHTCPPCPAPELLGGPSVFLFPPKPKDTLMISRTPEVTCVVVDVS |
| | | | HEDPEVKFNWYVDGVEVHNAKTKPCEEQYGSTYRCVSVLTVLHQDWLNGKEYKCKVSN |
| | | | KALPAPIEKTISKAKGQPREPQVYTLPPSREEMTKNQVSLTCLVKGFYPSDIAVEWES |
| | | | NGQPENNYKTTPPVLDSDGSFFLYSKLTVDKSRWQQGNVFSCSVMHEALHNHYTQKSL |
| | | | SLSPGK |
| 2373 | MA A103 CC | artificial | QVTLKESGPGILQPSQTLSLTCSFSGFSLNTSGMNVGWIRQPSGKCLDWLAHIWWNDD |
| | x I2C x | | KYYNPALKSRLTISKDTSNNQVFLKIASVVTADTATYYCVRSYFGDYVWYFDVWGAGT |
| | scFc-Full | | TVTVSSGGGGSGGGGSGGGGSDIQMTQTTSSLSASLGDRVTISCSASQGIHNYLNWYQ |
| | Sequence | | QKPDGTVKLLIYYTSSLHSGVPSRFSGSGSGTDYSLTISNLEPEDIATYFCQQYSKLP |
| | | | RTFGCGTKLEIKSGGGGSEVQLVESGGGLVQPGGSLKLSCAASGFTFNKYAMNWVRQA |
| | | | PGKGLEWVARIRSKYNNYATYYADSVKDRFTISRDDSKNTAYLQMNNLKTEDTAVYYC |
| | | | VRHGNFGNSYISYWAYWGQGTLVTVSSGGGGSGGGGSGGGGSQTVVTQEPSLTVSPGG |
| | | | TVTLTCGSSTGAVTSGNYPNWVQQKPGQAPRGLIGGTKFLAPGTPARFSGSLLGGKAA |
| | | | LTLSGVQPEDEAEYYCVLWYSNRWVFGGGTKLTVLGGGGDKTHTCPPCPAPELLGGPS |
| | | | VFLFPPKPKDTLMISRTPEVTCVVVDVSHEDPEVKENWYVDGVEVHNAKTKPCEEQYG |
| | | | STYRCVSVLTVLHQDWLNGKEYKCKVSNKALPAPIEKTISKAKGQPREPQVYTLPPSR |
| | | | EEMTKNQVSLTCLVKGFYPSDIAVEWESNGQPENNYKTTPPVLDSDGSFFLYSKLTVD |
| | | | KSRWQQGNVFSCSVMHEALHNHYTQKSLSLSPGKGGGGSGGGGSGGGGSGGGGSGGGG |
| | | | SGGGGSDKTHTCPPCPAPELLGGPSVFLFPPKPKDTLMISRTPEVTCVVVDVSHEDPE |
| | | | VKFNWYVDGVEVHNAKTKPCEEQYGSTYRCVSVLTVLHQDWLNGKEYKCKVSNKALPA |
| | | | PIEKTISKAKGQPREPQVYTLPPSREEMTKNQVSLTCLVKGFYPSDIAVEWESNGQPE |
| | | | NNYKTTPPVLDSDGSFFLYSKLTVDKSRWQQGNVFSCSVMHEALHNHYTQKSLSLSPG |
| | | | K |
| 2374 | MA A103 CC | artificial | QVTLKESGPGILQPSQTLSLTCSFSGFSLNTSGMNVGWIRQPSGKCLDWLAHIWWNDD |
| | x I2E x | | KYYNPALKSRLTISKDTSNNQVFLKIASVVTADTATYYCVRSYFGDYVWYFDVWGAGT |
| | scFc-Full | | TVTVSSGGGGSGGGGSGGGGSDIQMTQTTSSLSASLGDRVTISCSASQGIHNYLNWYQ |
| | Sequence | | QKPDGTVKLLIYYTSSLHSGVPSRFSGSGSGTDYSLTISNLEPEDIATYFCQQYSKLP |
| | | | RTFGCGTKLEIKSGGGGSEVQLVESGGGLVQPGGSLKLSCAASGFTENKYAINWVRQA |
| | | | PGKGLEWVARIRSKYNNYATYYADAVKDRFTISRDDSKNTVYLQMNNLKTEDTAVYYC |
| | | | ARAGNFGSSYISYWAYWGQGTLVTVSSGGGGSGGGGSGGGGSQTVVTQEPSLTVSPGG |
| | | | TVTITCGSSTGAVTSGNYPNWVQKKPGQAPRGLIGGTKFLAPGTPARFSGSLSGGKAA |
| | | | LTLSGVQPEDEAEYYCVLWYSNRWVFGSGTKLTVLGGGGDKTHTCPPCPAPELLGGPS |
| | | | VFLFPPKPKDTLMISRTPEVTCVVVDVSHEDPEVKENWYVDGVEVHNAKTKPCEEQYG |
| | | | STYRCVSVLTVLHQDWLNGKEYKCKVSNKALPAPIEKTISKAKGQPREPQVYTLPPSR |
| | | | EEMTKNQVSLTCLVKGFYPSDIAVEWESNGQPENNYKTTPPVLDSDGSFFLYSKLTVD |
| | | | KSRWQQGNVFSCSVMHEALHNHYTQKSLSLSPGKGGGGSGGGGSGGGGSGGGGSGGGG |
| | | | SGGGGSDKTHTCPPCPAPELLGGPSVFLFPPKPKDTLMISRTPEVTCVVVDVSHEDPE |
| | | | VKFNWYVDGVEVHNAKTKPCEEQYGSTYRCVSVLTVLHQDWLNGKEYKCKVSNKALPA |
| | | | PIEKTISKAKGQPREPQVYTLPPSREEMTKNQVSLTCLVKGFYPSDIAVEWESNGQPE |
| | | | NNYKTTPPVLDSDGSFFLYSKLTVDKSRWQQGNVFSCSVMHEALHNHYTQKSLSLSPG |
| | | | K |
| 2375 | MA A103 CC | artificial | QVTLKESGPGILQPSQTLSLTCSFSGFSLNTSGMNVGWIRQPSGKCLDWLAHIWWNDD |
| | x I2K x | | KYYNPALKSRLTISKDTSNNQVFLKIASVVTADTATYYCVRSYFGDYVWYFDVWGAGT |
| | scFc-Full | | TVTVSSGGGGSGGGGSGGGGSDIQMTQTTSSLSASLGDRVTISCSASQGIHNYLNWYQ |
| | Sequence | | QKPDGTVKLLIYYTSSLHSGVPSRFSGSGSGTDYSLTISNLEPEDIATYFCQQYSKLP |
| | | | RTFGCGTKLEIKSGGGGSEVQLVESGGGLVQPGGSLKLSCAASGFTFNKYAMNWVRQA |
| | | | PGKGLEWVARIRSKYNNYATYYAEAVKGRFTISRDDSKNTVYLQMNNLKTEDTAVYYC |
| | | | VRNENIGTSYISYWAYWGQGTLVTVSSGGGGSGGGGSGGGGSQTVVTQEPSLTVSPGG |
| | | | TVTITCGSSTGAVTSGNYPNWVQQKPGQAPRGLIGGTKFLAPGTPARFSGSLSGGKAA |
| | | | LTLSGVQPEDEAEYYCVLWYSNRWVFGSGTKLTVLGGGGDKTHTCPPCPAPELLGGPS |
| | | | VFLFPPKPKDTLMISRTPEVTCVVVDVSHEDPEVKENWYVDGVEVHNAKTKPCEEQYG |
| | | | STYRCVSVLTVLHQDWLNGKEYKCKVSNKALPAPIEKTISKAKGQPREPQVYTLPPSR |
| | | | EEMTKNQVSLTCLVKGFYPSDIAVEWESNGQPENNYKTTPPVLDSDGSFFLYSKLTVD |
| | | | KSRWQQGNVFSCSVMHEALHNHYTQKSLSLSPGKGGGGSGGGGSGGGGSGGGGSGGGG |
| | | | SGGGGSDKTHTCPPCPAPELLGGPSVFLFPPKPKDTLMISRTPEVTCVVVDVSHEDPE |
| | | | VKFNWYVDGVEVHNAKTKPCEEQYGSTYRCVSVLTVLHQDWLNGKEYKCKVSNKALPA |
| | | | PIEKTISKAKGQPREPQVYTLPPSREEMTKNQVSLTCLVKGFYPSDIAVEWESNGQPE |
| | | | NNYKTTPPVLDSDGSFFLYSKLTVDKSRWQQGNVFSCSVMHEALHNHYTQKSLSLSPG |
| | | | K |
| 2376 | MA A103 CC | artificial | QVTLKESGPGILQPSQTLSLTCSFSGFSLNTSGMNVGWIRQPSGKCLDWLAHIWWNDD |
| | x I2L x | | KYYNPALKSRLTISKDTSNNQVFLKIASVVTADTATYYCVRSYFGDYVWYFDVWGAGT |
| | scFc-Full | | TVTVSSGGGGSGGGGSGGGGSDIQMTQTTSSLSASLGDRVTISCSASQGIHNYLNWYQ |
| | Sequence | | QKPDGTVKLLIYYTSSLHSGVPSRFSGSGSGTDYSLTISNLEPEDIATYFCQQYSKLP |
| | | | RTFGCGTKLEIKSGGGGSEVQLVESGGGLVQPGGSLKLSCAASGFTFNKYAMNWVRQA |
| | | | PGKGMEWVARIRSKYNNYATYYADAVKDRFTISRDDSKNTLYLQMNNLKTEDTAVYYC |
| | | | VRAGNFGSSYISYFAYWGQGTLVTVSSGGGGSGGGGSGGGGSQTVVTQEPSLTVSPGG |
| | | | TVTITCGSSTGAVTSGNYPNWIQKKPGQAPRGLIGGTKFLAPGTPARFSGSLEGGKAA |
| | | | LTLSGVQPEDEAEYYCVLYYSNRWVFGSGTKLTVLGGGGDKTHTCPPCPAPELLGGPS |
| | | | VFLFPPKPKDTLMISRTPEVTCVVVDVSHEDPEVKENWYVDGVEVHNAKTKPCEEQYG |
| | | | STYRCVSVLTVLHQDWLNGKEYKCKVSNKALPAPIEKTISKAKGQPREPQVYTLPPSR |
| | | | EEMTKNQVSLTCLVKGFYPSDIAVEWESNGQPENNYKTTPPVLDSDGSFFLYSKLTVD |
| | | | KSRWQQGNVFSCSVMHEALHNHYTQKSLSLSPGKGGGGSGGGGSGGGGSGGGGSGGGG |
| | | | SGGGGSDKTHTCPPCPAPELLGGPSVFLFPPKPKDTLMISRTPEVTCVVVDVSHEDPE |
| | | | VKFNWYVDGVEVHNAKTKPCEEQYGSTYRCVSVLTVLHQDWLNGKEYKCKVSNKALPA |
| | | | PIEKTISKAKGQPREPQVYTLPPSREEMTKNQVSLTCLVKGFYPSDIAVEWESNGQPE |
| | | | NNYKTTPPVLDSDGSFFLYSKLTVDKSRWQQGNVFSCSVMHEALHNHYTQKSLSLSPG |
| | | | K |
| 2377 | MA A103 CC | artificial | QVTLKESGPGILQPSQTLSLTCSFSGFSLNTSGMNVGWIRQPSGKCLDWLAHIWWNDD |
| | x I2M x | | KYYNPALKSRLTISKDTSNNQVFLKIASVVTADTATYYCVRSYFGDYVWYFDVWGAGT |
| | scFc-Full | | TVTVSSGGGGSGGGGSGGGGSDIQMTQTTSSLSASLGDRVTISCSASQGIHNYLNWYQ |
| | Sequence | | QKPDGTVKLLIYYTSSLHSGVPSRFSGSGSGTDYSLTISNLEPEDIATYFCQQYSKLP |
| | | | RTFGCGTKLEIKSGGGGSEVQLVESGGGLVQPGGSLKLSCAASGFTFNKYAMNWVRQA |
| | | | PGKGMEWVARIRSKYNNYATYYADAVKDRFTISRDDSKNTLYLQMNNLKTEDTAVYYC |
| | | | VRAGNFGTSYISYWAYWGQGTLVTVSSGGGGSGGGGSGGGGSQTVVTQEPSLTVSPGG |
| | | | TVTITCGSSTGAVTSGNYPNWVQKKPGQAPRGLIGGTKFLAPGTPARFSGSLSGGKAA |
| | | | LTLSGVQPEDEAEYYCVLWYSNRWVFGSGTKLTVLGGGGDKTHTCPPCPAPELLGGPS |
| | | | VFLFPPKPKDTLMISRTPEVTCVVVDVSHEDPEVKENWYVDGVEVHNAKTKPCEEQYG |
| | | | STYRCVSVLTVLHQDWLNGKEYKCKVSNKALPAPIEKTISKAKGQPREPQVYTLPPSR |
| | | | EEMTKNQVSLTCLVKGFYPSDIAVEWESNGQPENNYKTTPPVLDSDGSFFLYSKLTVD |
| | | | KSRWQQGNVFSCSVMHEALHNHYTQKSLSLSPGKGGGGSGGGGSGGGGSGGGGSGGGG |
| | | | SGGGGSDKTHTCPPCPAPELLGGPSVFLFPPKPKDTLMISRTPEVTCVVVDVSHEDPE |
| | | | VKFNWYVDGVEVHNAKTKPCEEQYGSTYRCVSVLTVLHQDWLNGKEYKCKVSNKALPA |
| | | | PIEKTISKAKGQPREPQVYTLPPSREEMTKNQVSLTCLVKGFYPSDIAVEWESNGQPE |
| | | | NNYKTTPPVLDSDGSFFLYSKLTVDKSRWQQGNVFSCSVMHEALHNHYTQKSLSLSPG |
| | | | K |
| | | | QVTLKESGPGILQPSQTLSLTCSFSGFSLNTSGMNVGWIRQPSGKCLDWLAHIWWNDD |
| 2378 | MA A103 CC | artificial | KYYNPALKSRLTISKDTSNNQVFLKIASVVTADTATYYCVRSYFGDYVWYFDVWGAGT |
| | x I2M2 x | | TVTVSSGGGGSGGGGSGGGGSDIQMTQTTSSLSASLGDRVTISCSASQGIHNYLNWYQ |
| | scFc-Full | | QKPDGTVKLLIYYTSSLHSGVPSRFSGSGSGTDYSLTISNLEPEDIATYFCQQYSKLP |
| | Sequence | | RTFGCGTKLEIKSGGGGSEVQLVESGGGLVQPGGSLKLSCAASGFTENKYAINWVREA |
| | | | PGKGLEWVARIRSKYNNYATYYADAVKDRFTISRDDSKNTAYLQMNNLKTEDTAVYYC |
| | | | VRNANFGTSYISYFAYWGQGTLVTVSSGGGGSGGGGSGGGGSQTVVTQEPSLTVSPGG |
| | | | TVTLTCGSSTGAVTSGNYPNWVQKKPGQAPRGLIGGTKFLAPGTPARFSGSLLGGKAA |
| | | | LTLSGVQPEDEAEYYCVLWYSNRWVFGSGTKLTVLGGGGDKTHTCPPCPAPELLGGPS |
| | | | VFLFPPKPKDTLMISRTPEVTCVVVDVSHEDPEVKENWYVDGVEVHNAKTKPCEEQYG |
| | | | STYRCVSVLTVLHQDWLNGKEYKCKVSNKALPAPIEKTISKAKGQPREPQVYTLPPSR |
| | | | EEMTKNQVSLTCLVKGFYPSDIAVEWESNGQPENNYKTTPPVLDSDGSFFLYSKLTVD |
| | | | KSRWQQGNVFSCSVMHEALHNHYTQKSLSLSPGKGGGGSGGGGSGGGGSGGGGSGGGG |
| | | | SGGGGSDKTHTCPPCPAPELLGGPSVFLFPPKPKDTLMISRTPEVTCVVVDVSHEDPE |
| | | | VKFNWYVDGVEVHNAKTKPCEEQYGSTYRCVSVLTVLHQDWLNGKEYKCKVSNKALPA |
| | | | PIEKTISKAKGQPREPQVYTLPPSREEMTKNQVSLTCLVKGFYPSDIAVEWESNGQPE |
| | | | NNYKTTPPVLDSDGSFFLYSKLTVDKSRWQQGNVFSCSVMHEALHNHYTQKSLSLSPG |
| | | | K |
| 2379 | MART A103 | artificial | TSGMNVG |
| | CC-HCDR1 | | |
| 2380 | MART A103 | artificial | HIWWNDDKYYNPALKS |
| | CC-HCDR2 | | |
| 2381 | MART A103 | artificial | SYFGDYVWYFDV |
| | CC-HCDR3 | | |
| 2382 | MART A103 | artificial | SASQGIHNYLN |
| | CC-LCDR1 | | |
| 2383 | MART A103 | artificial | YTSSLHS |
| | CC-LCDR2 | | |
| 2384 | MART A103 | artificial | QQYSKLPRT |
| | CC-LCDR3 | | |
| 2385 | MART A103 | artificial | QVTLKESGPGILQPSQTLSLTCSFSGFSLNTSGMNVGWIRQPSGKCLDWLAHIWWNDD |
| | CC-VH | | KYYNPALKSRLTISKDTSNNQVFLKIASVVTADTATYYCVRSYFGDYVWYFDVWGAGT |
| | | | TVTVSS |
| 2386 | MART A103 | artificial | DIQMTQTTSSLSASLGDRVTISCSASQGIHNYLNWYQQKPDGTVKLLIYYTSSLHSGV |
| | CC-VL | | PSRFSGSGSGTDYSLTISNLEPEDIATYFCQQYSKLPRTFGCGTKLEIK |
| 2387 | MART A103 | artificial | EIQMTQTTSSLSASLGDRVTISCSASQGIHNYLNWYQQKPDGTVKLLIYYTSSLHSGV |
| | CC EI GQ- | | PSRFSGSGSGTDYSLTISNLEPEDIATYFCQQYSKLPRTFGCGTKLEIK |
| | VL | | |
| 2388 | MS 15-B12 | artificial | SSSYFWG |
| | CC-HCDR1 | | |
| 2389 | MS 15-B12 | artificial | NIYYSGSSNYNPSLKS |
| | CC-HCDR2 | | |
| 2390 | MS 15-B12 | artificial | LPRGDRDAFDI |
| | CC-HCDR3 | | |
| 2391 | MS 15-B12 | artificial | RASQGISNYLA |
| | CC-LCDR1 | | |
| 2392 | MS 15-B12 | artificial | AASTLQS |
| | CC-LCDR2 | | |
| 2393 | MS 15-B12 | artificial | QQSYSTPFT |
| | CC-LCDR3 | | |
| 2394 | MS 15-B12 | artificial | QVQLQESGPGLVKPSETLSLTCTVSGGSISSSSYFWGWIRQPPGKCLEWIGNIYYSGS |
| | CC-VH | | SNYNPSLKSRVTISVDTSKNQFSLKLSSVTAADTAVYYCARLPRGDRDAFDIWGQGTM |
| | | | VTVSS |
| 2395 | MS 15-B12 | artificial | DIVMTQSPSSLSASVGDRVTITCRASQGISNYLAWYQQKPGKVPKLLIYAASTLQSGV |
| | CC-VL | | PSRFSGSGSGTDFTLTISSLQPEDFATYYCQQSYSTPFTFGCGTKVEIK |
| 2396 | MS 15-B12 | artificial | EIVMTQSPSSLSASVGDRVTITCRASQGISNYLAWYQQKPGKVPKLLIYAASTLQSGV |
| | CC EI GQ- | | PSRFSGSGSGTDFTLTISSLQPEDFATYYCQQSYSTPFTFGCGTKVEIK |
| | VL | | |
| 2397 | MS 5-F11 | artificial | DYYMT |
| | HCDR1 | | |
| 2398 | MS 5-F11 | artificial | YISSSGSTIYYADSVKG |
| | HCDR2 | | |
| 2399 | MS 5-F11 | artificial | DRNSHFDY |
| | HCDR3 | | |
| 2400 | MS 5-F11 | artificial | RASQGINTWLA |
| | LCDR1 | | |
| 2401 | MS 5-F11 | artificial | GASGLQS |
| | LCDR2 | | |
| 2402 | MS 5-F11- | artificial | QQAKSFPRT |
| | LCDR3 | | |
| 2403 | MS 5-F11 | artificial | QVQLVESGGGLVKPGGSLRLSCAASGFTFSDYYMTWIRQAPGKGLEWLSYISSSGSTI |
| | VH | | YYADSVKGRFTISRDNAKNSLFLQMNSLRAEDTAVYYCARDRNSHFDYWGQGTLVTVS |
| | | | S |
| 2404 | MS 5-F11 | artificial | DIQMTQSPSSVSASVGDRVTITCRASQGINTWLAWYQQKPGKAPKLLIYGASGLQSGV |
| | VL | | PSRFSGSGSGTDFTLTISSLQPEDFATYYCQQAKSFPRTFGQGTKVEIK |
| 2405 | MS 5-F11 EI | artificial | EIQMTQSPSSVSASVGDRVTITCRASQGINTWLAWYQQKPGKAPKLLIYGASGLQSGV |
| | GQ-VL | | PSRFSGSGSGTDFTLTISSLQPEDFATYYCQQAKSFPRTFGQGTKVEIK |
| 2406 | MS R4L CC- | artificial | GYYIH |
| | HCDR1 | | |
| 2407 | MS R4L CC- | artificial | WINPNSGGTNYAQKFQG |
| | HCDR2 | | |
| 2408 | MS R4L CC- | artificial | VEAVAGREYYYFSGMDV |
| | HCDR3 | | |
| 2409 | MS R4L CC- | artificial | SGEKLGDKYVY |
| | LCDR1 | | |
| 2410 | MS R4L CC- | artificial | QSTKRPS |
| | LCDR2 | | |
| 2411 | MS R4L CC- | artificial | QAYHASTAV |
| | LCDR3 | | |
| 2412 | MS R4L CC- | artificial | QVQLVQSGAEVKKPGASVKVSCKASGYTFTGYYIHWVRQAPGQCLEWMGWINPNSGGT |
| | VH | | NYAQKFQGRVTMTRDTSISTAYMELSRLRSDDTAVYYCARVEAVAGREYYYFSGMDVW |
| | | | GQGTTVTVSS |
| 2413 | MS R4L CC- | artificial | SYELTQPPSVSVSPGQTASITCSGEKLGDKYVYWYQQKPGQSPVLVIYQSTKRPSGVP |
| | VL | | ERFSGSNSGNTATLTISGTQAMDEADYYCQAYHASTAVFGCGTKLTVL |
| 2414 | MS 15-B12 | artificial | QVQLQESGPGLVKPSETLSLTCTVSGGSISSSSYFWGWIRQPPGKCLEWIGNIYYSGS |
| | CC x I2C x | | SNYNPSLKSRVTISVDTSKNQFSLKLSSVTAADTAVYYCARLPRGDRDAFDIWGQGTM |
| | scFc-Full | | VTVSSGGGGSGGGGSGGGGSDIVMTQSPSSLSASVGDRVTITCRASQGISNYLAWYQQ |
| | Sequence | | KPGKVPKLLIYAASTLQSGVPSRFSGSGSGTDFTLTISSLQPEDFATYYCQQSYSTPF |
| | | | TFGCGTKVEIKSGGGGSEVQLVESGGGLVQPGGSLKLSCAASGFTENKYAMNWVRQAP |
| | | | GKGLEWVARIRSKYNNYATYYADSVKDRFTISRDDSKNTAYLQMNNLKTEDTAVYYCV |
| | | | RHGNFGNSYISYWAYWGQGTLVTVSSGGGGSGGGGSGGGGSQTVVTQEPSLTVSPGGT |
| | | | VTLTCGSSTGAVTSGNYPNWVQQKPGQAPRGLIGGTKFLAPGTPARFSGSLLGGKAAL |
| | | | TLSGVQPEDEAEYYCVLWYSNRWVFGGGTKLTVLGGGGDKTHTCPPCPAPELLGGPSV |
| | | | FLFPPKPKDTLMISRTPEVTCVVVDVSHEDPEVKENWYVDGVEVHNAKTKPCEEQYGS |
| | | | TYRCVSVLTVLHQDWLNGKEYKCKVSNKALPAPIEKTISKAKGQPREPQVYTLPPSRE |
| | | | EMTKNQVSLTCLVKGFYPSDIAVEWESNGQPENNYKTTPPVLDSDGSFFLYSKLTVDK |
| | | | SRWQQGNVFSCSVMHEALHNHYTQKSLSLSPGKGGGGSGGGGSGGGGSGGGGSGGGGS |
| | | | GGGGSDKTHTCPPCPAPELLGGPSVFLFPPKPKDTLMISRTPEVTCVVVDVSHEDPEV |
| | | | KFNWYVDGVEVHNAKTKPCEEQYGSTYRCVSVLTVLHQDWLNGKEYKCKVSNKALPAP |
| | | | IEKTISKAKGQPREPQVYTLPPSREEMTKNQVSLTCLVKGFYPSDIAVEWESNGQPEN |
| | | | NYKTTPPVLDSDGSFFLYSKLTVDKSRWQQGNVFSCSVMHEALHNHYTQKSLSLSPGK |
| 2415 | MS 15-B12 | artificial | QVQLQESGPGLVKPSETLSLTCTVSGGSISSSSYFWGWIRQPPGKCLEWIGNIYYSGS |
| | CC x I2E x | | SNYNPSLKSRVTISVDTSKNQFSLKLSSVTAADTAVYYCARLPRGDRDAFDIWGQGTM |
| | ScFc-Full | | VTVSSGGGGSGGGGSGGGGSDIVMTQSPSSLSASVGDRVTITCRASQGISNYLAWYQQ |
| | Sequence | | KPGKVPKLLIYAASTLQSGVPSRFSGSGSGTDFTLTISSLQPEDFATYYCQQSYSTPF |
| | | | TFGCGTKVEIKSGGGGSEVQLVESGGGLVQPGGSLKLSCAASGFTENKYAINWVRQAP |
| | | | GKGLEWVARIRSKYNNYATYYADAVKDRFTISRDDSKNTVYLQMNNLKTEDTAVYYCA |
| | | | RAGNFGSSYISYWAYWGQGTLVTVSSGGGGSGGGGSGGGGSQTVVTQEPSLTVSPGGT |
| | | | VTITCGSSTGAVTSGNYPNWVQKKPGQAPRGLIGGTKFLAPGTPARFSGSLSGGKAAL |
| | | | TLSGVQPEDEAEYYCVLWYSNRWVFGSGTKLTVLGGGGDKTHTCPPCPAPELLGGPSV |
| | | | FLFPPKPKDTLMISRTPEVTCVVVDVSHEDPEVKENWYVDGVEVHNAKTKPCEEQYGS |
| | | | TYRCVSVLTVLHQDWLNGKEYKCKVSNKALPAPIEKTISKAKGQPREPQVYTLPPSRE |
| | | | EMTKNQVSLTCLVKGFYPSDIAVEWESNGQPENNYKTTPPVLDSDGSFFLYSKLTVDK |
| | | | SRWQQGNVFSCSVMHEALHNHYTQKSLSLSPGKGGGGSGGGGSGGGGSGGGGSGGGGS |
| | | | GGGGSDKTHTCPPCPAPELLGGPSVFLFPPKPKDTLMISRTPEVTCVVVDVSHEDPEV |
| | | | KFNWYVDGVEVHNAKTKPCEEQYGSTYRCVSVLTVLHQDWLNGKEYKCKVSNKALPAP |
| | | | IEKTISKAKGQPREPQVYTLPPSREEMTKNQVSLTCLVKGFYPSDIAVEWESNGQPEN |
| | | | NYKTTPPVLDSDGSFFLYSKLTVDKSRWQQGNVFSCSVMHEALHNHYTQKSLSLSPGK |
| 2416 | MS 15-B12 | artificial | QVQLQESGPGLVKPSETLSLTCTVSGGSISSSSYFWGWIRQPPGKCLEWIGNIYYSGS |
| | CC x I2K x | | SNYNPSLKSRVTISVDTSKNQFSLKLSSVTAADTAVYYCARLPRGDRDAFDIWGQGTM |
| | scFc-Full | | VTVSSGGGGSGGGGSGGGGSDIVMTQSPSSLSASVGDRVTITCRASQGISNYLAWYQQ |
| | Sequence | | KPGKVPKLLIYAASTLQSGVPSRFSGSGSGTDFTLTISSLQPEDFATYYCQQSYSTPF |
| | | | TFGCGTKVEIKSGGGGSEVQLVESGGGLVQPGGSLKLSCAASGFTFNKYAMNWVRQAP |
| | | | GKGLEWVARIRSKYNNYATYYAEAVKGRFTISRDDSKNTVYLQMNNLKTEDTAVYYCV |
| | | | RNENIGTSYISYWAYWGQGTLVTVSSGGGGSGGGGSGGGGSQTVVTQEPSLTVSPGGT |
| | | | VTITCGSSTGAVTSGNYPNWVQQKPGQAPRGLIGGTKFLAPGTPARFSGSLSGGKAAL |
| | | | TLSGVQPEDEAEYYCVLWYSNRWVFGSGTKLTVLGGGGDKTHTCPPCPAPELLGGPSV |
| | | | FLFPPKPKDTLMISRTPEVTCVVVDVSHEDPEVKENWYVDGVEVHNAKTKPCEEQYGS |
| | | | TYRCVSVLTVLHQDWLNGKEYKCKVSNKALPAPIEKTISKAKGQPREPQVYTLPPSRE |
| | | | EMTKNQVSLTCLVKGFYPSDIAVEWESNGQPENNYKTTPPVLDSDGSFFLYSKLTVDK |
| | | | SRWQQGNVFSCSVMHEALHNHYTQKSLSLSPGKGGGGSGGGGSGGGGSGGGGSGGGGS |
| | | | GGGGSDKTHTCPPCPAPELLGGPSVFLFPPKPKDTLMISRTPEVTCVVVDVSHEDPEV |
| | | | KFNWYVDGVEVHNAKTKPCEEQYGSTYRCVSVLTVLHQDWLNGKEYKCKVSNKALPAP |
| | | | IEKTISKAKGQPREPQVYTLPPSREEMTKNQVSLTCLVKGFYPSDIAVEWESNGQPEN |
| | | | NYKTTPPVLDSDGSFFLYSKLTVDKSRWQQGNVFSCSVMHEALHNHYTQKSLSLSPGK |
| 2417 | MS 15-B12 | artificial | QVQLQESGPGLVKPSETLSLTCTVSGGSISSSSYFWGWIRQPPGKCLEWIGNIYYSGS |
| | CC x I2L x | | SNYNPSLKSRVTISVDTSKNQFSLKLSSVTAADTAVYYCARLPRGDRDAFDIWGQGTM |
| | scFc-Full | | VTVSSGGGGSGGGGSGGGGSDIVMTQSPSSLSASVGDRVTITCRASQGISNYLAWYQQ |
| | Sequence | | KPGKVPKLLIYAASTLQSGVPSRFSGSGSGTDFTLTISSLQPEDFATYYCQQSYSTPF |
| | | | TFGCGTKVEIKSGGGGSEVQLVESGGGLVQPGGSLKLSCAASGFTFNKYAMNWVRQAP |
| | | | GKGMEWVARIRSKYNNYATYYADAVKDRFTISRDDSKNTLYLQMNNLKTEDTAVYYCV |
| | | | RAGNFGSSYISYFAYWGQGTLVTVSSGGGGSGGGGSGGGGSQTVVTQEPSLTVSPGGT |
| | | | VTITCGSSTGAVTSGNYPNWIQKKPGQAPRGLIGGTKFLAPGTPARFSGSLEGGKAAL |
| | | | TLSGVQPEDEAEYYCVLYYSNRWVFGSGTKLTVLGGGGDKTHTCPPCPAPELLGGPSV |
| | | | FLFPPKPKDTLMISRTPEVTCVVVDVSHEDPEVKENWYVDGVEVHNAKTKPCEEQYGS |
| | | | TYRCVSVLTVLHQDWLNGKEYKCKVSNKALPAPIEKTISKAKGQPREPQVYTLPPSRE |
| | | | EMTKNQVSLTCLVKGFYPSDIAVEWESNGQPENNYKTTPPVLDSDGSFFLYSKLTVDK |
| | | | SRWQQGNVFSCSVMHEALHNHYTQKSLSLSPGKGGGGSGGGGSGGGGSGGGGSGGGGS |
| | | | GGGGSDKTHTCPPCPAPELLGGPSVFLFPPKPKDTLMISRTPEVTCVVVDVSHEDPEV |
| | | | KFNWYVDGVEVHNAKTKPCEEQYGSTYRCVSVLTVLHQDWLNGKEYKCKVSNKALPAP |
| | | | IEKTISKAKGQPREPQVYTLPPSREEMTKNQVSLTCLVKGFYPSDIAVEWESNGQPEN |
| | | | NYKTTPPVLDSDGSFFLYSKLTVDKSRWQQGNVFSCSVMHEALHNHYTQKSLSLSPGK |
| 2418 | MS 15-B12 | artificial | QVQLQESGPGLVKPSETLSLTCTVSGGSISSSSYFWGWIRQPPGKCLEWIGNIYYSGS |
| | CC x I2M x | | SNYNPSLKSRVTISVDTSKNQFSLKLSSVTAADTAVYYCARLPRGDRDAFDIWGQGTM |
| | ScFc-Full | | VTVSSGGGGSGGGGSGGGGSDIVMTQSPSSLSASVGDRVTITCRASQGISNYLAWYQQ |
| | Sequence | | KPGKVPKLLIYAASTLQSGVPSRFSGSGSGTDFTLTISSLQPEDFATYYCQQSYSTPF |
| | | | TFGCGTKVEIKSGGGGSEVQLVESGGGLVQPGGSLKLSCAASGFTFNKYAMNWVRQAP |
| | | | GKGMEWVARIRSKYNNYATYYADAVKDRFTISRDDSKNTLYLQMNNLKTEDTAVYYCV |
| | | | RAGNFGTSYISYWAYWGQGTLVTVSSGGGGSGGGGSGGGGSQTVVTQEPSLTVSPGGT |
| | | | VTITCGSSTGAVTSGNYPNWVQKKPGQAPRGLIGGTKFLAPGTPARFSGSLSGGKAAL |
| | | | TLSGVQPEDEAEYYCVLWYSNRWVFGSGTKLTVLGGGGDKTHTCPPCPAPELLGGPSV |
| | | | FLFPPKPKDTLMISRTPEVTCVVVDVSHEDPEVKFNWYVDGVEVHNAKTKPCEEQYGS |
| | | | TYRCVSVLTVLHQDWLNGKEYKCKVSNKALPAPIEKTISKAKGQPREPQVYTLPPSRE |
| | | | EMTKNQVSLTCLVKGFYPSDIAVEWESNGQPENNYKTTPPVLDSDGSFFLYSKLTVDK |
| | | | SRWQQGNVFSCSVMHEALHNHYTQKSLSLSPGKGGGGSGGGGSGGGGSGGGGSGGGGS |
| | | | GGGGSDKTHTCPPCPAPELLGGPSVFLFPPKPKDTLMISRTPEVTCVVVDVSHEDPEV |
| | | | KFNWYVDGVEVHNAKTKPCEEQYGSTYRCVSVLTVLHQDWLNGKEYKCKVSNKALPAP |
| | | | IEKTISKAKGQPREPQVYTLPPSREEMTKNQVSLTCLVKGFYPSDIAVEWESNGQPEN |
| | | | NYKTTPPVLDSDGSFFLYSKLTVDKSRWQQGNVFSCSVMHEALHNHYTQKSLSLSPGK |
| 2419 | MS 15-B12 | artificial | QVQLQESGPGLVKPSETLSLTCTVSGGSISSSSYFWGWIRQPPGKCLEWIGNIYYSGS |
| | CC x I2M2 x | | SNYNPSLKSRVTISVDTSKNQFSLKLSSVTAADTAVYYCARLPRGDRDAFDIWGQGTM |
| | scFc-Full | | VTVSSGGGGSGGGGSGGGGSDIVMTQSPSSLSASVGDRVTITCRASQGISNYLAWYQQ |
| | Sequence | | KPGKVPKLLIYAASTLQSGVPSRFSGSGSGTDFTLTISSLQPEDFATYYCQQSYSTPF |
| | | | TFGCGTKVEIKSGGGGSEVQLVESGGGLVQPGGSLKLSCAASGFTFNKYAINWVREAP |
| | | | GKGLEWVARIRSKYNNYATYYADAVKDRFTISRDDSKNTAYLQMNNLKTEDTAVYYCV |
| | | | RNANFGTSYISYFAYWGQGTLVTVSSGGGGSGGGGSGGGGSQTVVTQEPSLTVSPGGT |
| | | | VTLTCGSSTGAVTSGNYPNWVQKKPGQAPRGLIGGTKFLAPGTPARFSGSLLGGKAAL |
| | | | TLSGVQPEDEAEYYCVLWYSNRWVFGSGTKLTVLGGGGDKTHTCPPCPAPELLGGPSV |
| | | | FLFPPKPKDTLMISRTPEVTCVVVDVSHEDPEVKENWYVDGVEVHNAKTKPCEEQYGS |
| | | | TYRCVSVLTVLHQDWLNGKEYKCKVSNKALPAPIEKTISKAKGQPREPQVYTLPPSRE |
| | | | EMTKNQVSLTCLVKGFYPSDIAVEWESNGQPENNYKTTPPVLDSDGSFFLYSKLTVDK |
| | | | SRWQQGNVFSCSVMHEALHNHYTQKSLSLSPGKGGGGSGGGGSGGGGSGGGGSGGGGS |
| | | | GGGGSDKTHTCPPCPAPELLGGPSVFLFPPKPKDTLMISRTPEVTCVVVDVSHEDPEV |
| | | | KFNWYVDGVEVHNAKTKPCEEQYGSTYRCVSVLTVLHQDWLNGKEYKCKVSNKALPAP |
| | | | IEKTISKAKGQPREPQVYTLPPSREEMTKNQVSLTCLVKGFYPSDIAVEWESNGQPEN |
| | | | NYKTTPPVLDSDGSFFLYSKLTVDKSRWQQGNVFSCSVMHEALHNHYTQKSLSLSPGK |
| 2420 | MS 5-F11 x | artificial | QVQLVESGGGLVKPGGSLRLSCAASGFTFSDYYMTWIRQAPGKGLEWLSYISSSGSTI |
| | I2Cx scFc- | | YYADSVKGRFTISRDNAKNSLFLQMNSLRAEDTAVYYCARDRNSHFDYWGQGTLVTVS |
| | Full | | SGGGGSGGGGSGGGGSDIQMTQSPSSVSASVGDRVTITCRASQGINTWLAWYQQKPGK |
| | Sequence | | APKLLIYGASGLQSGVPSRFSGSGSGTDFTLTISSLQPEDFATYYCQQAKSFPRTFGQ |
| | | | GTKVEIKSGGGGSEVQLVESGGGLVQPGGSLKLSCAASGFTFNKYAMNWVRQAPGKGL |
| | | | EWVARIRSKYNNYATYYADSVKDRFTISRDDSKNTAYLQMNNLKTEDTAVYYCVRHGN |
| | | | FGNSYISYWAYWGQGTLVTVSSGGGGSGGGGSGGGGSQTVVTQEPSLTVSPGGTVTLT |
| | | | CGSSTGAVTSGNYPNWVQQKPGQAPRGLIGGTKFLAPGTPARFSGSLLGGKAALTLSG |
| | | | VQPEDEAEYYCVLWYSNRWVFGGGTKLTVLGGGGDKTHTCPPCPAPELLGGPSVFLFP |
| | | | PKPKDTLMISRTPEVTCVVVDVSHEDPEVKFNWYVDGVEVHNAKTKPCEEQYGSTYRC |
| | | | VSVLTVLHQDWLNGKEYKCKVSNKALPAPIEKTISKAKGQPREPQVYTLPPSREEMTK |
| | | | NQVSLTCLVKGFYPSDIAVEWESNGQPENNYKTTPPVLDSDGSFFLYSKLTVDKSRWQ |
| | | | QGNVFSCSVMHEALHNHYTQKSLSLSPGKGGGGSGGGGSGGGGSGGGGSGGGGSGGGG |
| | | | SDKTHTCPPCPAPELLGGPSVFLFPPKPKDTLMISRTPEVTCVVVDVSHEDPEVKENW |
| | | | YVDGVEVHNAKTKPCEEQYGSTYRCVSVLTVLHQDWLNGKEYKCKVSNKALPAPIEKT |
| | | | ISKAKGQPREPQVYTLPPSREEMTKNQVSLTCLVKGFYPSDIAVEWESNGQPENNYKT |
| | | | TPPVLDSDGSFFLYSKLTVDKSRWQQGNVFSCSVMHEALHNHYTQKSLSLSPGK |
| 2421 | MS 5-F11 x | artificial | QVQLVESGGGLVKPGGSLRLSCAASGFTFSDYYMTWIRQAPGKGLEWLSYISSSGSTI |
| | I2Ex scFc- | | YYADSVKGRFTISRDNAKNSLFLQMNSLRAEDTAVYYCARDRNSHFDYWGQGTLVTVS |
| | Full | | SGGGGSGGGGSGGGGSDIQMTQSPSSVSASVGDRVTITCRASQGINTWLAWYQQKPGK |
| | Sequence | | APKLLIYGASGLQSGVPSRFSGSGSGTDFTLTISSLQPEDFATYYCQQAKSFPRTFGQ |
| | | | GTKVEIKSGGGGSEVQLVESGGGLVQPGGSLKLSCAASGFTFNKYAINWVRQAPGKGL |
| | | | EWVARIRSKYNNYATYYADAVKDRFTISRDDSKNTVYLQMNNLKTEDTAVYYCARAGN |
| | | | FGSSYISYWAYWGQGTLVTVSSGGGGSGGGGSGGGGSQTVVTQEPSLTVSPGGTVTIT |
| | | | CGSSTGAVTSGNYPNWVQKKPGQAPRGLIGGTKFLAPGTPARFSGSLSGGKAALTLSG |
| | | | VQPEDEAEYYCVLWYSNRWVFGSGTKLTVLGGGGDKTHTCPPCPAPELLGGPSVFLFP |
| | | | PKPKDTLMISRTPEVTCVVVDVSHEDPEVKFNWYVDGVEVHNAKTKPCEEQYGSTYRC |
| | | | VSVLTVLHQDWLNGKEYKCKVSNKALPAPIEKTISKAKGQPREPQVYTLPPSREEMTK |
| | | | NQVSLTCLVKGFYPSDIAVEWESNGQPENNYKTTPPVLDSDGSFFLYSKLTVDKSRWQ |
| | | | QGNVFSCSVMHEALHNHYTQKSLSLSPGKGGGGSGGGGSGGGGSGGGGSGGGGSGGGG |
| | | | SDKTHTCPPCPAPELLGGPSVFLFPPKPKDTLMISRTPEVTCVVVDVSHEDPEVKENW |
| | | | YVDGVEVHNAKTKPCEEQYGSTYRCVSVLTVLHQDWLNGKEYKCKVSNKALPAPIEKT |
| | | | ISKAKGQPREPQVYTLPPSREEMTKNQVSLTCLVKGFYPSDIAVEWESNGQPENNYKT |
| | | | TPPVLDSDGSFFLYSKLTVDKSRWQQGNVFSCSVMHEALHNHYTQKSLSLSPGK |
| 2422 | MS 5-F11 x | artificial | QVQLVESGGGLVKPGGSLRLSCAASGFTFSDYYMTWIRQAPGKGLEWLSYISSSGSTI |
| | I2Kx scFc- | | YYADSVKGRFTISRDNAKNSLFLQMNSLRAEDTAVYYCARDRNSHFDYWGQGTLVTVS |
| | Full | | SGGGGSGGGGSGGGGSDIQMTQSPSSVSASVGDRVTITCRASQGINTWLAWYQQKPGK |
| | Sequence | | APKLLIYGASGLQSGVPSRFSGSGSGTDFTLTISSLQPEDFATYYCQQAKSFPRTFGQ |
| | | | GTKVEIKSGGGGSEVQLVESGGGLVQPGGSLKLSCAASGFTFNKYAMNWVRQAPGKGL |
| | | | EWVARIRSKYNNYATYYAEAVKGRFTISRDDSKNTVYLQMNNLKTEDTAVYYCVRNEN |
| | | | IGTSYISYWAYWGQGTLVTVSSGGGGSGGGGSGGGGSQTVVTQEPSLTVSPGGTVTIT |
| | | | CGSSTGAVTSGNYPNWVQQKPGQAPRGLIGGTKFLAPGTPARFSGSLSGGKAALTLSG |
| | | | VQPEDEAEYYCVLWYSNRWVFGSGTKLTVLGGGGDKTHTCPPCPAPELLGGPSVFLFP |
| | | | PKPKDTLMISRTPEVTCVVVDVSHEDPEVKENWYVDGVEVHNAKTKPCEEQYGSTYRC |
| | | | VSVLTVLHQDWLNGKEYKCKVSNKALPAPIEKTISKAKGQPREPQVYTLPPSREEMTK |
| | | | NQVSLTCLVKGFYPSDIAVEWESNGQPENNYKTTPPVLDSDGSFFLYSKLTVDKSRWQ |
| | | | QGNVFSCSVMHEALHNHYTQKSLSLSPGKGGGGSGGGGSGGGGSGGGGSGGGGSGGGG |
| | | | SDKTHTCPPCPAPELLGGPSVFLFPPKPKDTLMISRTPEVTCVVVDVSHEDPEVKENW |
| | | | YVDGVEVHNAKTKPCEEQYGSTYRCVSVLTVLHQDWLNGKEYKCKVSNKALPAPIEKT |
| | | | ISKAKGQPREPQVYTLPPSREEMTKNQVSLTCLVKGFYPSDIAVEWESNGQPENNYKT |
| | | | TPPVLDSDGSFFLYSKLTVDKSRWQQGNVFSCSVMHEALHNHYTQKSLSLSPGK |
| 2423 | MS 5-F11 x | artificial | QVQLVESGGGLVKPGGSLRLSCAASGFTFSDYYMTWIRQAPGKGLEWLSYISSSGSTI |
| | I2Lx scFc- | | YYADSVKGRFTISRDNAKNSLFLQMNSLRAEDTAVYYCARDRNSHFDYWGQGTLVTVS |
| | Full | | SGGGGSGGGGSGGGGSDIQMTQSPSSVSASVGDRVTITCRASQGINTWLAWYQQKPGK |
| | Sequence | | APKLLIYGASGLQSGVPSRFSGSGSGTDFTLTISSLQPEDFATYYCQQAKSFPRTFGQ |
| | | | GTKVEIKSGGGGSEVQLVESGGGLVQPGGSLKLSCAASGFTFNKYAMNWVRQAPGKGM |
| | | | EWVARIRSKYNNYATYYADAVKDRFTISRDDSKNTLYLQMNNLKTEDTAVYYCVRAGN |
| | | | FGSSYISYFAYWGQGTLVTVSSGGGGSGGGGSGGGGSQTVVTQEPSLTVSPGGTVTIT |
| | | | CGSSTGAVTSGNYPNWIQKKPGQAPRGLIGGTKFLAPGTPARFSGSLEGGKAALTLSG |
| | | | VQPEDEAEYYCVLYYSNRWVFGSGTKLTVLGGGGDKTHTCPPCPAPELLGGPSVFLFP |
| | | | PKPKDTLMISRTPEVTCVVVDVSHEDPEVKFNWYVDGVEVHNAKTKPCEEQYGSTYRC |
| | | | VSVLTVLHQDWLNGKEYKCKVSNKALPAPIEKTISKAKGQPREPQVYTLPPSREEMTK |
| | | | NQVSLTCLVKGFYPSDIAVEWESNGQPENNYKTTPPVLDSDGSFFLYSKLTVDKSRWQ |
| | | | QGNVFSCSVMHEALHNHYTQKSLSLSPGKGGGGSGGGGSGGGGSGGGGSGGGGSGGGG |
| | | | SDKTHTCPPCPAPELLGGPSVFLFPPKPKDTLMISRTPEVTCVVVDVSHEDPEVKENW |
| | | | YVDGVEVHNAKTKPCEEQYGSTYRCVSVLTVLHQDWLNGKEYKCKVSNKALPAPIEKT |
| | | | ISKAKGQPREPQVYTLPPSREEMTKNQVSLTCLVKGFYPSDIAVEWESNGQPENNYKT |
| | | | TPPVLDSDGSFFLYSKLTVDKSRWQQGNVFSCSVMHEALHNHYTQKSLSLSPGK |
| 2424 | MS 5-F11 x | artificial | QVQLVESGGGLVKPGGSLRLSCAASGFTFSDYYMTWIRQAPGKGLEWLSYISSSGSTI |
| | I2M2x scFc | | YYADSVKGRFTISRDNAKNSLFLQMNSLRAEDTAVYYCARDRNSHFDYWGQGTLVTVS |
| | -Full | | SGGGGSGGGGSGGGGSDIQMTQSPSSVSASVGDRVTITCRASQGINTWLAWYQQKPGK |
| | Sequence | | APKLLIYGASGLQSGVPSRFSGSGSGTDFTLTISSLQPEDFATYYCQQAKSFPRTFGQ |
| | | | GTKVEIKSGGGGSEVQLVESGGGLVQPGGSLKLSCAASGFTFNKYAINWVREAPGKGL |
| | | | EWVARIRSKYNNYATYYADAVKDRFTISRDDSKNTAYLQMNNLKTEDTAVYYCVRNAN |
| | | | FGTSYISYFAYWGQGTLVTVSSGGGGSGGGGSGGGGSQTVVTQEPSLTVSPGGTVTLT |
| | | | CGSSTGAVTSGNYPNWVQKKPGQAPRGLIGGTKFLAPGTPARFSGSLLGGKAALTLSG |
| | | | VQPEDEAEYYCVLWYSNRWVFGSGTKLTVLGGGGDKTHTCPPCPAPELLGGPSVFLFP |
| | | | PKPKDTLMISRTPEVTCVVVDVSHEDPEVKENWYVDGVEVHNAKTKPCEEQYGSTYRC |
| | | | VSVLTVLHQDWLNGKEYKCKVSNKALPAPIEKTISKAKGQPREPQVYTLPPSREEMTK |
| | | | NQVSLTCLVKGFYPSDIAVEWESNGQPENNYKTTPPVLDSDGSFFLYSKLTVDKSRWQ |
| | | | QGNVFSCSVMHEALHNHYTQKSLSLSPGKGGGGSGGGGSGGGGSGGGGSGGGGSGGGG |
| | | | SDKTHTCPPCPAPELLGGPSVFLFPPKPKDTLMISRTPEVTCVVVDVSHEDPEVKENW |
| | | | YVDGVEVHNAKTKPCEEQYGSTYRCVSVLTVLHQDWLNGKEYKCKVSNKALPAPIEKT |
| | | | ISKAKGQPREPQVYTLPPSREEMTKNQVSLTCLVKGFYPSDIAVEWESNGQPENNYKT |
| | | | TPPVLDSDGSFFLYSKLTVDKSRWQQGNVFSCSVMHEALHNHYTQKSLSLSPGK |
| 2425 | MS 5-F11 x | artificial | QVQLVESGGGLVKPGGSLRLSCAASGFTFSDYYMTWIRQAPGKGLEWLSYISSSGSTI |
| | I2Mx scFc- | | YYADSVKGRFTISRDNAKNSLFLQMNSLRAEDTAVYYCARDRNSHFDYWGQGTLVTVS |
| | Full | | SGGGGSGGGGSGGGGSDIQMTQSPSSVSASVGDRVTITCRASQGINTWLAWYQQKPGK |
| | Sequence | | APKLLIYGASGLQSGVPSRFSGSGSGTDFTLTISSLQPEDFATYYCQQAKSFPRTFGQ |
| | | | GTKVEIKSGGGGSEVQLVESGGGLVQPGGSLKLSCAASGFTFNKYAMNWVRQAPGKGM |
| | | | EWVARIRSKYNNYATYYADAVKDRFTISRDDSKNTLYLQMNNLKTEDTAVYYCVRAGN |
| | | | FGTSYISYWAYWGQGTLVTVSSGGGGSGGGGSGGGGSQTVVTQEPSLTVSPGGTVTIT |
| | | | CGSSTGAVTSGNYPNWVQKKPGQAPRGLIGGTKFLAPGTPARFSGSLSGGKAALTLSG |
| | | | VQPEDEAEYYCVLWYSNRWVFGSGTKLTVLGGGGDKTHTCPPCPAPELLGGPSVFLFP |
| | | | PKPKDTLMISRTPEVTCVVVDVSHEDPEVKENWYVDGVEVHNAKTKPCEEQYGSTYRC |
| | | | VSVLTVLHQDWLNGKEYKCKVSNKALPAPIEKTISKAKGQPREPQVYTLPPSREEMTK |
| | | | NQVSLTCLVKGFYPSDIAVEWESNGQPENNYKTTPPVLDSDGSFFLYSKLTVDKSRWQ |
| | | | QGNVFSCSVMHEALHNHYTQKSLSLSPGKGGGGSGGGGSGGGGSGGGGSGGGGSGGGG |
| | | | SDKTHTCPPCPAPELLGGPSVFLFPPKPKDTLMISRTPEVTCVVVDVSHEDPEVKENW |
| | | | YVDGVEVHNAKTKPCEEQYGSTYRCVSVLTVLHQDWLNGKEYKCKVSNKALPAPIEKT |
| | | | ISKAKGQPREPQVYTLPPSREEMTKNQVSLTCLVKGFYPSDIAVEWESNGQPENNYKT |
| | | | TPPVLDSDGSFFLYSKLTVDKSRWQQGNVFSCSVMHEALHNHYTQKSLSLSPGK |
| 2426 | MS R4L CC x | artificial | QVQLVQSGAEVKKPGASVKVSCKASGYTFTGYYIHWVRQAPGQCLEWMGWINPNSGGT |
| | I2C x scFc | | NYAQKFQGRVTMTRDTSISTAYMELSRLRSDDTAVYYCARVEAVAGREYYYFSGMDVW |
| | -Full | | GQGTTVTVSSGGGGSGGGGSGGGGSSYELTQPPSVSVSPGQTASITCSGEKLGDKYVY |
| | Sequence | | WYQQKPGQSPVLVIYQSTKRPSGVPERFSGSNSGNTATLTISGTQAMDEADYYCQAYH |
| | | | ASTAVFGCGTKLTVLSGGGGSEVQLVESGGGLVQPGGSLKLSCAASGFTFNKYAMNWV |
| | | | RQAPGKGLEWVARIRSKYNNYATYYADSVKDRFTISRDDSKNTAYLQMNNLKTEDTAV |
| | | | YYCVRHGNFGNSYISYWAYWGQGTLVTVSSGGGGSGGGGSGGGGSQTVVTQEPSLTVS |
| | | | PGGTVTLTCGSSTGAVTSGNYPNWVQQKPGQAPRGLIGGTKFLAPGTPARFSGSLLGG |
| | | | KAALTLSGVQPEDEAEYYCVLWYSNRWVFGGGTKLTVLGGGGDKTHTCPPCPAPELLG |
| | | | GPSVFLFPPKPKDTLMISRTPEVTCVVVDVSHEDPEVKENWYVDGVEVHNAKTKPCEE |
| | | | QYGSTYRCVSVLTVLHQDWLNGKEYKCKVSNKALPAPIEKTISKAKGQPREPQVYTLP |
| | | | PSREEMTKNQVSLTCLVKGFYPSDIAVEWESNGQPENNYKTTPPVLDSDGSFFLYSKL |
| | | | TVDKSRWQQGNVFSCSVMHEALHNHYTQKSLSLSPGKGGGGSGGGGSGGGGSGGGGSG |
| | | | GGGSGGGGSDKTHTCPPCPAPELLGGPSVFLFPPKPKDTLMISRTPEVTCVVVDVSHE |
| | | | DPEVKFNWYVDGVEVHNAKTKPCEEQYGSTYRCVSVLTVLHQDWLNGKEYKCKVSNKA |
| | | | LPAPIEKTISKAKGQPREPQVYTLPPSREEMTKNQVSLTCLVKGFYPSDIAVEWESNG |
| | | | QPENNYKTTPPVLDSDGSFFLYSKLTVDKSRWQQGNVFSCSVMHEALHNHYTQKSLSL |
| | | | SPGK |
| 2427 | MS R4L CC x | artificial | QVQLVQSGAEVKKPGASVKVSCKASGYTFTGYYIHWVRQAPGQCLEWMGWINPNSGGT |
| | I2E x scFc | | NYAQKFQGRVTMTRDTSISTAYMELSRLRSDDTAVYYCARVEAVAGREYYYFSGMDVW |
| | -Full | | GQGTTVTVSSGGGGSGGGGSGGGGSSYELTQPPSVSVSPGQTASITCSGEKLGDKYVY |
| | Sequence | | WYQQKPGQSPVLVIYQSTKRPSGVPERFSGSNSGNTATLTISGTQAMDEADYYCQAYH |
| | | | ASTAVFGCGTKLTVLSGGGGSEVQLVESGGGLVQPGGSLKLSCAASGFTFNKYAINWV |
| | | | RQAPGKGLEWVARIRSKYNNYATYYADAVKDRFTISRDDSKNTVYLQMNNLKTEDTAV |
| | | | YYCARAGNFGSSYISYWAYWGQGTLVTVSSGGGGSGGGGSGGGGSQTVVTQEPSLTVS |
| | | | PGGTVTITCGSSTGAVTSGNYPNWVQKKPGQAPRGLIGGTKFLAPGTPARFSGSLSGG |
| | | | KAALTLSGVQPEDEAEYYCVLWYSNRWVFGSGTKLTVLGGGGDKTHTCPPCPAPELLG |
| | | | GPSVFLFPPKPKDTLMISRTPEVTCVVVDVSHEDPEVKENWYVDGVEVHNAKTKPCEE |
| | | | QYGSTYRCVSVLTVLHQDWLNGKEYKCKVSNKALPAPIEKTISKAKGQPREPQVYTLP |
| | | | PSREEMTKNQVSLTCLVKGFYPSDIAVEWESNGQPENNYKTTPPVLDSDGSFFLYSKL |
| | | | TVDKSRWQQGNVFSCSVMHEALHNHYTQKSLSLSPGKGGGGSGGGGSGGGGSGGGGSG |
| | | | GGGSGGGGSDKTHTCPPCPAPELLGGPSVFLFPPKPKDTLMISRTPEVTCVVVDVSHE |
| | | | DPEVKFNWYVDGVEVHNAKTKPCEEQYGSTYRCVSVLTVLHQDWLNGKEYKCKVSNKA |
| | | | LPAPIEKTISKAKGQPREPQVYTLPPSREEMTKNQVSLTCLVKGFYPSDIAVEWESNG |
| | | | QPENNYKTTPPVLDSDGSFFLYSKLTVDKSRWQQGNVFSCSVMHEALHNHYTQKSLSL |
| | | | SPGK |
| 2428 | MS R4L CC x | artificial | QVQLVQSGAEVKKPGASVKVSCKASGYTFTGYYIHWVRQAPGQCLEWMGWINPNSGGT |
| | I2K x scFc | | NYAQKFQGRVTMTRDTSISTAYMELSRLRSDDTAVYYCARVEAVAGREYYYFSGMDVW |
| | -Full | | GQGTTVTVSSGGGGSGGGGSGGGGSSYELTQPPSVSVSPGQTASITCSGEKLGDKYVY |
| | Sequence | | WYQQKPGQSPVLVIYQSTKRPSGVPERFSGSNSGNTATLTISGTQAMDEADYYCQAYH |
| | | | ASTAVFGCGTKLTVLSGGGGSEVQLVESGGGLVQPGGSLKLSCAASGFTENKYAMNWV |
| | | | RQAPGKGLEWVARIRSKYNNYATYYAEAVKGRFTISRDDSKNTVYLQMNNLKTEDTAV |
| | | | YYCVRNENIGTSYISYWAYWGQGTLVTVSSGGGGSGGGGSGGGGSQTVVTQEPSLTVS |
| | | | PGGTVTITCGSSTGAVTSGNYPNWVQQKPGQAPRGLIGGTKFLAPGTPARFSGSLSGG |
| | | | KAALTLSGVQPEDEAEYYCVLWYSNRWVFGSGTKLTVLGGGGDKTHTCPPCPAPELLG |
| | | | GPSVFLFPPKPKDTLMISRTPEVTCVVVDVSHEDPEVKFNWYVDGVEVHNAKTKPCEE |
| | | | QYGSTYRCVSVLTVLHQDWLNGKEYKCKVSNKALPAPIEKTISKAKGQPREPQVYTLP |
| | | | PSREEMTKNQVSLTCLVKGFYPSDIAVEWESNGQPENNYKTTPPVLDSDGSFFLYSKL |
| | | | TVDKSRWQQGNVFSCSVMHEALHNHYTQKSLSLSPGKGGGGSGGGGSGGGGSGGGGSG |
| | | | GGGSGGGGSDKTHTCPPCPAPELLGGPSVFLFPPKPKDTLMISRTPEVTCVVVDVSHE |
| | | | DPEVKFNWYVDGVEVHNAKTKPCEEQYGSTYRCVSVLTVLHQDWLNGKEYKCKVSNKA |
| | | | LPAPIEKTISKAKGQPREPQVYTLPPSREEMTKNQVSLTCLVKGFYPSDIAVEWESNG |
| | | | QPENNYKTTPPVLDSDGSFFLYSKLTVDKSRWQQGNVFSCSVMHEALHNHYTQKSLSL |
| | | | SPGK |
| 2429 | MS R4L CC x | artificial | QVQLVQSGAEVKKPGASVKVSCKASGYTFTGYYIHWVRQAPGQCLEWMGWINPNSGGT |
| | I2L x scFc | | NYAQKFQGRVTMTRDTSISTAYMELSRLRSDDTAVYYCARVEAVAGREYYYFSGMDVW |
| | Full | | GQGTTVTVSSGGGGSGGGGSGGGGSSYELTQPPSVSVSPGQTASITCSGEKLGDKYVY |
| | Sequence | | WYQQKPGQSPVLVIYQSTKRPSGVPERFSGSNSGNTATLTISGTQAMDEADYYCQAYH |
| | | | ASTAVFGCGTKLTVLSGGGGSEVQLVESGGGLVQPGGSLKLSCAASGFTENKYAMNWV |
| | | | RQAPGKGMEWVARIRSKYNNYATYYADAVKDRFTISRDDSKNTLYLQMNNLKTEDTAV |
| | | | YYCVRAGNFGSSYISYFAYWGQGTLVTVSSGGGGSGGGGSGGGGSQTVVTQEPSLTVS |
| | | | PGGTVTITCGSSTGAVTSGNYPNWIQKKPGQAPRGLIGGTKFLAPGTPARFSGSLEGG |
| | | | KAALTLSGVQPEDEAEYYCVLYYSNRWVFGSGTKLTVLGGGGDKTHTCPPCPAPELLG |
| | | | GPSVFLFPPKPKDTLMISRTPEVTCVVVDVSHEDPEVKENWYVDGVEVHNAKTKPCEE |
| | | | QYGSTYRCVSVLTVLHQDWLNGKEYKCKVSNKALPAPIEKTISKAKGQPREPQVYTLP |
| | | | PSREEMTKNQVSLTCLVKGFYPSDIAVEWESNGQPENNYKTTPPVLDSDGSFFLYSKL |
| | | | TVDKSRWQQGNVFSCSVMHEALHNHYTQKSLSLSPGKGGGGSGGGGSGGGGSGGGGSG |
| | | | GGGSGGGGSDKTHTCPPCPAPELLGGPSVFLFPPKPKDTLMISRTPEVTCVVVDVSHE |
| | | | DPEVKFNWYVDGVEVHNAKTKPCEEQYGSTYRCVSVLTVLHQDWLNGKEYKCKVSNKA |
| | | | LPAPIEKTISKAKGQPREPQVYTLPPSREEMTKNQVSLTCLVKGFYPSDIAVEWESNG |
| | | | QPENNYKTTPPVLDSDGSFFLYSKLTVDKSRWQQGNVFSCSVMHEALHNHYTQKSLSL |
| | | | SPGK |
| 2430 | MS R4L CC x | artificial | QVQLVQSGAEVKKPGASVKVSCKASGYTFTGYYIHWVRQAPGQCLEWMGWINPNSGGT |
| | I2M x scFc | | NYAQKFQGRVTMTRDTSISTAYMELSRLRSDDTAVYYCARVEAVAGREYYYFSGMDVW |
| | -Full | | GQGTTVTVSSGGGGSGGGGSGGGGSSYELTQPPSVSVSPGQTASITCSGEKLGDKYVY |
| | Sequence | | WYQQKPGQSPVLVIYQSTKRPSGVPERFSGSNSGNTATLTISGTQAMDEADYYCQAYH |
| | | | ASTAVFGCGTKLTVLSGGGGSEVQLVESGGGLVQPGGSLKLSCAASGFTENKYAMNWV |
| | | | RQAPGKGMEWVARIRSKYNNYATYYADAVKDRFTISRDDSKNTLYLQMNNLKTEDTAV |
| | | | YYCVRAGNFGTSYISYWAYWGQGTLVTVSSGGGGSGGGGSGGGGSQTVVTQEPSLTVS |
| | | | PGGTVTITCGSSTGAVTSGNYPNWVQKKPGQAPRGLIGGTKFLAPGTPARFSGSLSGG |
| | | | KAALTLSGVQPEDEAEYYCVLWYSNRWVFGSGTKLTVLGGGGDKTHTCPPCPAPELLG |
| | | | GPSVFLFPPKPKDTLMISRTPEVTCVVVDVSHEDPEVKENWYVDGVEVHNAKTKPCEE |
| | | | QYGSTYRCVSVLTVLHQDWLNGKEYKCKVSNKALPAPIEKTISKAKGQPREPQVYTLP |
| | | | PSREEMTKNQVSLTCLVKGFYPSDIAVEWESNGQPENNYKTTPPVLDSDGSFFLYSKL |
| | | | TVDKSRWQQGNVFSCSVMHEALHNHYTQKSLSLSPGKGGGGSGGGGSGGGGSGGGGSG |
| | | | GGGSGGGGSDKTHTCPPCPAPELLGGPSVFLFPPKPKDTLMISRTPEVTCVVVDVSHE |
| | | | DPEVKFNWYVDGVEVHNAKTKPCEEQYGSTYRCVSVLTVLHQDWLNGKEYKCKVSNKA |
| | | | LPAPIEKTISKAKGQPREPQVYTLPPSREEMTKNQVSLTCLVKGFYPSDIAVEWESNG |
| | | | QPENNYKTTPPVLDSDGSFFLYSKLTVDKSRWQQGNVFSCSVMHEALHNHYTQKSLSL |
| | | | SPGK |
| 2431 | MS R4L CC x | artificial | QVQLVQSGAEVKKPGASVKVSCKASGYTFTGYYIHWVRQAPGQCLEWMGWINPNSGGT |
| | I2M2 x scFc | | NYAQKFQGRVTMTRDTSISTAYMELSRLRSDDTAVYYCARVEAVAGREYYYFSGMDVW |
| | -Full | | GQGTTVTVSSGGGGSGGGGSGGGGSSYELTQPPSVSVSPGQTASITCSGEKLGDKYVY |
| | Sequence | | WYQQKPGQSPVLVIYQSTKRPSGVPERFSGSNSGNTATLTISGTQAMDEADYYCQAYH |
| | | | ASTAVFGCGTKLTVLSGGGGSEVQLVESGGGLVQPGGSLKLSCAASGFTENKYAINWV |
| | | | REAPGKGLEWVARIRSKYNNYATYYADAVKDRFTISRDDSKNTAYLQMNNLKTEDTAV |
| | | | YYCVRNANFGTSYISYFAYWGQGTLVTVSSGGGGSGGGGSGGGGSQTVVTQEPSLTVS |
| | | | PGGTVTLTCGSSTGAVTSGNYPNWVQKKPGQAPRGLIGGTKFLAPGTPARFSGSLLGG |
| | | | KAALTLSGVQPEDEAEYYCVLWYSNRWVFGSGTKLTVLGGGGDKTHTCPPCPAPELLG |
| | | | GPSVFLFPPKPKDTLMISRTPEVTCVVVDVSHEDPEVKENWYVDGVEVHNAKTKPCEE |
| | | | QYGSTYRCVSVLTVLHQDWLNGKEYKCKVSNKALPAPIEKTISKAKGQPREPQVYTLP |
| | | | PSREEMTKNQVSLTCLVKGFYPSDIAVEWESNGQPENNYKTTPPVLDSDGSFFLYSKL |
| | | | TVDKSRWQQGNVFSCSVMHEALHNHYTQKSLSLSPGKGGGGSGGGGSGGGGSGGGGSG |
| | | | GGGSGGGGSDKTHTCPPCPAPELLGGPSVFLFPPKPKDTLMISRTPEVTCVVVDVSHE |
| | | | DPEVKFNWYVDGVEVHNAKTKPCEEQYGSTYRCVSVLTVLHQDWLNGKEYKCKVSNKA |
| | | | LPAPIEKTISKAKGQPREPQVYTLPPSREEMTKNQVSLTCLVKGFYPSDIAVEWESNG |
| | | | QPENNYKTTPPVLDSDGSFFLYSKLTVDKSRWQQGNVFSCSVMHEALHNHYTQKSLSI |
| | | | SPGK |
| 2432 | MU 8-B7 CC | artificial | GYYWS |
| | -HCDR1 | | |
| 2433 | MU 8-B7 CC | artificial | DIDASGSTKYNPSLKS |
| | -HCDR2 | | |
| 2434 | MU 8-B7 CC | artificial | KKYSTVWSYFDN |
| | -HCDR3 | | |
| 2435 | MU 8-B7 CC | artificial | SGDKLGDKYAS |
| | -LCDR1 | | |
| 2436 | MU 8-B7 CC | artificial | QDRKRPS |
| | LCDR2 | | |
| 2437 | MU 8-B7 CC | artificial | QAWGSSTAV |
| | -LCDR3 | | |
| 2438 | MU 8-B7 CC | artificial | QVQLQQWGAGLLKPSETLSLTCAVYGGSFSGYYWSWIRQPPGKCLEWIGDIDASGSTK |
| | -VH | | YNPSLKSRVTISLDTSKNQFSLKLNSVTAADTAVYFCARKKYSTVWSYFDNWGQGTLV |
| | | | TVSS |
| 2439 | MU 8-B7 CC | artificial | SYELTQPSSVSVPPGQTASITCSGDKLGDKYASWYQQKPGQSPVLVIYQDRKRPSGVP |
| | -VL | | ERFSGSNSGNTATLTISGTQAMDEADYYCQAWGSSTAVFGCGTKLTVL |
| 2440 | MU 8-B7 CC | artificial | QVQLQQWGAGLLKPSETLSLTCAVYGGSFSGYYWSWIRQPPGKCLEWIGDIDASGSTK |
| | x I2Cx scFc | | YNPSLKSRVTISLDTSKNQFSLKLNSVTAADTAVYFCARKKYSTVWSYFDNWGQGTLV |
| | -Full | | TVSSGGGGSGGGGSGGGGSSYELTQPSSVSVPPGQTASITCSGDKLGDKYASWYQQKP |
| | Sequence | | GQSPVLVIYQDRKRPSGVPERFSGSNSGNTATLTISGTQAMDEADYYCQAWGSSTAVF |
| | | | GCGTKLTVLSGGGGSEVQLVESGGGLVQPGGSLKLSCAASGFTFNKYAMNWVRQAPGK |
| | | | GLEWVARIRSKYNNYATYYADSVKDRFTISRDDSKNTAYLQMNNLKTEDTAVYYCVRH |
| | | | GNFGNSYISYWAYWGQGTLVTVSSGGGGSGGGGSGGGGSQTVVTQEPSLTVSPGGTVT |
| | | | LTCGSSTGAVTSGNYPNWVQQKPGQAPRGLIGGTKFLAPGTPARFSGSLLGGKAALTL |
| | | | SGVQPEDEAEYYCVLWYSNRWVFGGGTKLTVLGGGGDKTHTCPPCPAPELLGGPSVEL |
| | | | FPPKPKDTLMISRTPEVTCVVVDVSHEDPEVKENWYVDGVEVHNAKTKPCEEQYGSTY |
| | | | RCVSVLTVLHQDWLNGKEYKCKVSNKALPAPIEKTISKAKGQPREPQVYTLPPSREEM |
| | | | TKNQVSLTCLVKGFYPSDIAVEWESNGQPENNYKTTPPVLDSDGSFFLYSKLTVDKSR |
| | | | WQQGNVFSCSVMHEALHNHYTQKSLSLSPGKGGGGSGGGGSGGGGSGGGGSGGGGSGG |
| | | | GGSDKTHTCPPCPAPELLGGPSVFLFPPKPKDTLMISRTPEVTCVVVDVSHEDPEVKF |
| | | | NWYVDGVEVHNAKTKPCEEQYGSTYRCVSVLTVLHQDWLNGKEYKCKVSNKALPAPIE |
| | | | KTISKAKGQPREPQVYTLPPSREEMTKNQVSLTCLVKGFYPSDIAVEWESNGQPENNY |
| | | | KTTPPVLDSDGSFFLYSKLTVDKSRWQQGNVFSCSVMHEALHNHYTQKSLSLSPGK |
| 2441 | MU 8-B7 CC | artificial | QVQLQQWGAGLLKPSETLSLTCAVYGGSFSGYYWSWIRQPPGKCLEWIGDIDASGSTK |
| | x I2Ex scFc | | YNPSLKSRVTISLDTSKNQFSLKLNSVTAADTAVYFCARKKYSTVWSYFDNWGQGTLV |
| | -Full | | TVSSGGGGSGGGGSGGGGSSYELTQPSSVSVPPGQTASITCSGDKLGDKYASWYQQKP |
| | Sequence | | GQSPVLVIYQDRKRPSGVPERFSGSNSGNTATLTISGTQAMDEADYYCQAWGSSTAVF |
| | | | GCGTKLTVLSGGGGSEVQLVESGGGLVQPGGSLKLSCAASGFTFNKYAINWVRQAPGK |
| | | | GLEWVARIRSKYNNYATYYADAVKDRFTISRDDSKNTVYLQMNNLKTEDTAVYYCARA |
| | | | GNFGSSYISYWAYWGQGTLVTVSSGGGGSGGGGSGGGGSQTVVTQEPSLTVSPGGTVT |
| | | | ITCGSSTGAVTSGNYPNWVQKKPGQAPRGLIGGTKFLAPGTPARFSGSLSGGKAALTL |
| | | | SGVQPEDEAEYYCVLWYSNRWVFGSGTKLTVLGGGGDKTHTCPPCPAPELLGGPSVFL |
| | | | FPPKPKDTLMISRTPEVTCVVVDVSHEDPEVKENWYVDGVEVHNAKTKPCEEQYGSTY |
| | | | RCVSVLTVLHQDWLNGKEYKCKVSNKALPAPIEKTISKAKGQPREPQVYTLPPSREEM |
| | | | TKNQVSLTCLVKGFYPSDIAVEWESNGQPENNYKTTPPVLDSDGSFFLYSKLTVDKSR |
| | | | WQQGNVFSCSVMHEALHNHYTQKSLSLSPGKGGGGSGGGGSGGGGSGGGGSGGGGSGG |
| | | | GGSDKTHTCPPCPAPELLGGPSVFLFPPKPKDTLMISRTPEVTCVVVDVSHEDPEVKF |
| | | | NWYVDGVEVHNAKTKPCEEQYGSTYRCVSVLTVLHQDWLNGKEYKCKVSNKALPAPIE |
| | | | KTISKAKGQPREPQVYTLPPSREEMTKNQVSLTCLVKGFYPSDIAVEWESNGQPENNY |
| | | | KTTPPVLDSDGSFFLYSKLTVDKSRWQQGNVFSCSVMHEALHNHYTQKSLSLSPGK |
| 2442 | MU 8-B7 CC | artificial | QVQLQQWGAGLLKPSETLSLTCAVYGGSFSGYYWSWIRQPPGKCLEWIGDIDASGSTK |
| | x I2Kx scFc | | YNPSLKSRVTISLDTSKNQFSLKLNSVTAADTAVYFCARKKYSTVWSYFDNWGQGTLV |
| | -Full | | TVSSGGGGSGGGGSGGGGSSYELTQPSSVSVPPGQTASITCSGDKLGDKYASWYQQKP |
| | Sequence | | GQSPVLVIYQDRKRPSGVPERFSGSNSGNTATLTISGTQAMDEADYYCQAWGSSTAVF |
| | | | GCGTKLTVLSGGGGSEVQLVESGGGLVQPGGSLKLSCAASGFTFNKYAMNWVRQAPGK |
| | | | GLEWVARIRSKYNNYATYYAEAVKGRFTISRDDSKNTVYLQMNNLKTEDTAVYYCVRN |
| | | | ENIGTSYISYWAYWGQGTLVTVSSGGGGSGGGGSGGGGSQTVVTQEPSLTVSPGGTVT |
| | | | ITCGSSTGAVTSGNYPNWVQQKPGQAPRGLIGGTKFLAPGTPARFSGSLSGGKAALTL |
| | | | SGVQPEDEAEYYCVLWYSNRWVFGSGTKLTVLGGGGDKTHTCPPCPAPELLGGPSVFL |
| | | | FPPKPKDTLMISRTPEVTCVVVDVSHEDPEVKFNWYVDGVEVHNAKTKPCEEQYGSTY |
| | | | RCVSVLTVLHQDWLNGKEYKCKVSNKALPAPIEKTISKAKGQPREPQVYTLPPSREEM |
| | | | TKNQVSLTCLVKGFYPSDIAVEWESNGQPENNYKTTPPVLDSDGSFFLYSKLTVDKSR |
| | | | WQQGNVFSCSVMHEALHNHYTQKSLSLSPGKGGGGSGGGGSGGGGSGGGGSGGGGSGG |
| | | | GGSDKTHTCPPCPAPELLGGPSVFLFPPKPKDTLMISRTPEVTCVVVDVSHEDPEVKF |
| | | | NWYVDGVEVHNAKTKPCEEQYGSTYRCVSVLTVLHQDWLNGKEYKCKVSNKALPAPIE |
| | | | KTISKAKGQPREPQVYTLPPSREEMTKNQVSLTCLVKGFYPSDIAVEWESNGQPENNY |
| | | | KTTPPVLDSDGSFFLYSKLTVDKSRWQQGNVFSCSVMHEALHNHYTQKSLSLSPGK |
| 2443 | MU 8-B7 CC | artificial | QVQLQQWGAGLLKPSETLSLTCAVYGGSFSGYYWSWIRQPPGKCLEWIGDIDASGSTK |
| | x I2Lx scFc | | YNPSLKSRVTISLDTSKNQFSLKLNSVTAADTAVYFCARKKYSTVWSYFDNWGQGTLV |
| | -Full | | TVSSGGGGSGGGGSGGGGSSYELTQPSSVSVPPGQTASITCSGDKLGDKYASWYQQKP |
| | Sequence | | GQSPVLVIYQDRKRPSGVPERFSGSNSGNTATLTISGTQAMDEADYYCQAWGSSTAVF |
| | | | GCGTKLTVLSGGGGSEVQLVESGGGLVQPGGSLKLSCAASGFTFNKYAMNWVRQAPGK |
| | | | GMEWVARIRSKYNNYATYYADAVKDRFTISRDDSKNTLYLQMNNLKTEDTAVYYCVRA |
| | | | GNFGSSYISYFAYWGQGTLVTVSSGGGGSGGGGSGGGGSQTVVTQEPSLTVSPGGTVT |
| | | | ITCGSSTGAVTSGNYPNWIQKKPGQAPRGLIGGTKFLAPGTPARFSGSLEGGKAALTL |
| | | | SGVQPEDEAEYYCVLYYSNRWVFGSGTKLTVLGGGGDKTHTCPPCPAPELLGGPSVFL |
| | | | FPPKPKDTLMISRTPEVTCVVVDVSHEDPEVKENWYVDGVEVHNAKTKPCEEQYGSTY |
| | | | RCVSVLTVLHQDWLNGKEYKCKVSNKALPAPIEKTISKAKGQPREPQVYTLPPSREEM |
| | | | TKNQVSLTCLVKGFYPSDIAVEWESNGQPENNYKTTPPVLDSDGSFFLYSKLTVDKSR |
| | | | WQQGNVFSCSVMHEALHNHYTQKSLSLSPGKGGGGSGGGGSGGGGSGGGGSGGGGSGG |
| | | | GGSDKTHTCPPCPAPELLGGPSVFLFPPKPKDTLMISRTPEVTCVVVDVSHEDPEVKF |
| | | | NWYVDGVEVHNAKTKPCEEQYGSTYRCVSVLTVLHQDWLNGKEYKCKVSNKALPAPIE |
| | | | KTISKAKGQPREPQVYTLPPSREEMTKNQVSLTCLVKGFYPSDIAVEWESNGQPENNY |
| | | | KTTPPVLDSDGSFFLYSKLTVDKSRWQQGNVFSCSVMHEALHNHYTQKSLSLSPGK |
| 2444 | MU 8-B7 CC | artificial | QVQLQQWGAGLLKPSETLSLTCAVYGGSFSGYYWSWIRQPPGKCLEWIGDIDASGSTK |
| | x I2M2x | | YNPSLKSRVTISLDTSKNQFSLKLNSVTAADTAVYFCARKKYSTVWSYFDNWGQGTLV |
| | scFc-Full | | TVSSGGGGSGGGGSGGGGSSYELTQPSSVSVPPGQTASITCSGDKLGDKYASWYQQKP |
| | Sequence | | GQSPVLVIYQDRKRPSGVPERFSGSNSGNTATLTISGTQAMDEADYYCQAWGSSTAVE |
| | | | GCGTKLTVLSGGGGSEVQLVESGGGLVQPGGSLKLSCAASGFTENKYAINWVREAPGK |
| | | | GLEWVARIRSKYNNYATYYADAVKDRFTISRDDSKNTAYLQMNNLKTEDTAVYYCVRN |
| | | | ANFGTSYISYFAYWGQGTLVTVSSGGGGSGGGGSGGGGSQTVVTQEPSLTVSPGGTVT |
| | | | LTCGSSTGAVTSGNYPNWVQKKPGQAPRGLIGGTKFLAPGTPARFSGSLLGGKAALTL |
| | | | SGVQPEDEAEYYCVLWYSNRWVFGSGTKLTVLGGGGDKTHTCPPCPAPELLGGPSVFL |
| | | | FPPKPKDTLMISRTPEVTCVVVDVSHEDPEVKFNWYVDGVEVHNAKTKPCEEQYGSTY |
| | | | RCVSVLTVLHQDWLNGKEYKCKVSNKALPAPIEKTISKAKGQPREPQVYTLPPSREEM |
| | | | TKNQVSLTCLVKGFYPSDIAVEWESNGQPENNYKTTPPVLDSDGSFFLYSKLTVDKSR |
| | | | WQQGNVFSCSVMHEALHNHYTQKSLSLSPGKGGGGSGGGGSGGGGSGGGGSGGGGSGG |
| | | | GGSDKTHTCPPCPAPELLGGPSVFLFPPKPKDTLMISRTPEVTCVVVDVSHEDPEVKF |
| | | | NWYVDGVEVHNAKTKPCEEQYGSTYRCVSVLTVLHQDWLNGKEYKCKVSNKALPAPIE |
| | | | KTISKAKGQPREPQVYTLPPSREEMTKNQVSLTCLVKGFYPSDIAVEWESNGQPENNY |
| | | | KTTPPVLDSDGSFFLYSKLTVDKSRWQQGNVFSCSVMHEALHNHYTQKSLSLSPGK |
| 2445 | MU 8-B7 CC | artificial | QVQLQQWGAGLLKPSETLSLTCAVYGGSFSGYYWSWIRQPPGKCLEWIGDIDASGSTK |
| | x I2Mx scFc | | YNPSLKSRVTISLDTSKNQFSLKLNSVTAADTAVYFCARKKYSTVWSYFDNWGQGTLV |
| | -Full | | TVSSGGGGSGGGGSGGGGSSYELTQPSSVSVPPGQTASITCSGDKLGDKYASWYQQKP |
| | Sequence | | GQSPVLVIYQDRKRPSGVPERFSGSNSGNTATLTISGTQAMDEADYYCQAWGSSTAVF |
| | | | GCGTKLTVLSGGGGSEVQLVESGGGLVQPGGSLKLSCAASGFTENKYAMNWVRQAPGK |
| | | | GMEWVARIRSKYNNYATYYADAVKDRFTISRDDSKNTLYLQMNNLKTEDTAVYYCVRA |
| | | | GNFGTSYISYWAYWGQGTLVTVSSGGGGSGGGGSGGGGSQTVVTQEPSLTVSPGGTVT |
| | | | ITCGSSTGAVTSGNYPNWVQKKPGQAPRGLIGGTKFLAPGTPARFSGSLSGGKAALTL |
| | | | SGVQPEDEAEYYCVLWYSNRWVFGSGTKLTVLGGGGDKTHTCPPCPAPELLGGPSVFL |
| | | | FPPKPKDTLMISRTPEVTCVVVDVSHEDPEVKFNWYVDGVEVHNAKTKPCEEQYGSTY |
| | | | RCVSVLTVLHQDWLNGKEYKCKVSNKALPAPIEKTISKAKGQPREPQVYTLPPSREEM |
| | | | TKNQVSLTCLVKGFYPSDIAVEWESNGQPENNYKTTPPVLDSDGSFFLYSKLTVDKSR |
| | | | WQQGNVFSCSVMHEALHNHYTQKSLSLSPGKGGGGSGGGGSGGGGSGGGGSGGGGSGG |
| | | | GGSDKTHTCPPCPAPELLGGPSVFLFPPKPKDTLMISRTPEVTCVVVDVSHEDPEVKE |
| | | | NWYVDGVEVHNAKTKPCEEQYGSTYRCVSVLTVLHQDWLNGKEYKCKVSNKALPAPIE |
| | | | KTISKAKGQPREPQVYTLPPSREEMTKNQVSLTCLVKGFYPSDIAVEWESNGQPENNY |
| | | | KTTPPVLDSDGSFFLYSKLTVDKSRWQQGNVFSCSVMHEALHNHYTQKSLSLSPGK |
| 2446 | PM 76-B10- | artificial | DYYMY |
| | HCDR1 | | |
| 2447 | PM 76-B10- | artificial | IISDGGYYTYYSDIIKG |
| | HCDR2 | | |
| 2448 | PM 76-B10- | artificial | GFPLLRHGAMDY |
| | HCDR3 | | |
| 2449 | PM 76-B10- | artificial | KASQNVDTNVA |
| | LCDR1 | | |
| 2450 | PM 76-B10- | artificial | SASYRYS |
| | LCDR2 | | |
| 2451 | PM 76-B10- | artificial | QQYDSYPYT |
| | LCDR3 | | |
| 2452 | PM 76-B10- | artificial | QVQLVESGGGLVKPGESLRLSCAASGFTFSDYYMYWVRQAPGKGLEWVAIISDGGYYT |
| | VH | | YYSDIIKGRFTISRDNAKNSLYLQMNSLKAEDTAVYYCARGFPLLRHGAMDYWGQGTL |
| | | | VTVSS |
| 2453 | PM 76-B10- | artificial | DIQMTQSPSSLSASVGDRVTITCKASQNVDTNVAWYQQKPGQAPKSLIYSASYRYSDV |
| | VL | | PSRFSGSASGTDFTLTISSVQSEDFATYYCQQYDSYPYTFGGGTKLEIK |
| 2454 | PM 76-B10 | artificial | EIQMTQSPSSLSASVGDRVTITCKASQNVDTNVAWYQQKPGQAPKSLIYSASYRYSDV |
| | EI GQ-VL | | PSRFSGSASGTDFTLTISSVQSEDFATYYCQQYDSYPYTFGGGTKLEIK |
| 2455 | PM 76- | artificial | DYYMY |
| | B10.11 CC- | | |
| | HCDR1 | | |
| 2456 | PM 76- | artificial | IISDGGYYTYYSDIIKG |
| | B10.11 CC- | | |
| | HCDR2 | | |
| 2457 | PM 76- | artificial | GFPLLRHGAMDY |
| | B10.11 CC- | | |
| | HCDR3 | | |
| 2458 | PM 76- | artificial | KASQNVDTNVA |
| | B10.11 CC- | | |
| | LCDR1 | | |
| 2459 | PM 76- | artificial | SASYVYW |
| | B10.11 CC- | | |
| | LCDR2 | | |
| 2460 | PM 76- | artificial | QQYDQQLIT |
| | B10.11 CC- | | |
| | LCDR3 | | |
| 2461 | PM 76- | artificial | QVQLVESGGGLVKPGESLRLSCAASGFTFSDYYMYWVRQAPGKCLEWVAIISDGGYYT |
| | B10.11 CC- | | YYSDIIKGRFTISRDNAKNSLYLQMNSLKAEDTAVYYCARGFPLLRHGAMDYWGQGTL |
| | VH | | VTVSS |
| 2462 | PM 76- | artificial | DIQMTQSPSSLSASVGDRVTITCKASQNVDTNVAWYQQKPGQAPKSLIYSASYVYWDV |
| | B10.11 CC- | | PSRFSGSASGTDFTLTISSVQSEDFATYYCQQYDQQLITFGCGTKLEIK |
| | VL | | |
| 2463 | PM 76- | artificial | EIQMTQSPSSLSASVGDRVTITCKASQNVDTNVAWYQQKPGQAPKSLIYSASYVYWDV |
| | B10.11 CC | | PSRFSGSASGTDFTLTISSVQSEDFATYYCQQYDQQLITFGCGTKLEIK |
| | EI GQ-VL | | |
| 2464 | PM 76-B10x | artificial | QVQLVESGGGLVKPGESLRLSCAASGFTFSDYYMYWVRQAPGKGLEWVAIISDGGYYT |
| | I2C-Full | | YYSDIIKGRFTISRDNAKNSLYLQMNSLKAEDTAVYYCARGFPLLRHGAMDYWGQGTL |
| | Sequence | | VTVSSGGGGSGGGGSGGGGSDIQMTQSPSSLSASVGDRVTITCKASQNVDTNVAWYQQ |
| | | | KPGQAPKSLIYSASYRYSDVPSRFSGSASGTDFTLTISSVQSEDFATYYCQQYDSYPY |
| | | | TFGGGTKLEIKSGGGGSEVQLVESGGGLVQPGGSLKLSCAASGFTENKYAMNWVRQAP |
| | | | GKGLEWVARIRSKYNNYATYYADSVKDRFTISRDDSKNTAYLQMNNLKTEDTAVYYCV |
| | | | RHGNFGNSYISYWAYWGQGTLVTVSSGGGGSGGGGSGGGGSQTVVTQEPSLTVSPGGT |
| | | | VTLTCGSSTGAVTSGNYPNWVQQKPGQAPRGLIGGTKFLAPGTPARFSGSLLGGKAAL |
| | | | TLSGVQPEDEAEYYCVLWYSNRWVFGGGTKLTVL |
| 2465 | PM 76-B10x | artificial | QVQLVESGGGLVKPGESLRLSCAASGFTFSDYYMYWVRQAPGKGLEWVAIISDGGYYT |
| | I2E-Full | | YYSDIIKGRFTISRDNAKNSLYLQMNSLKAEDTAVYYCARGFPLLRHGAMDYWGQGTL |
| | Sequence | | VTVSSGGGGSGGGGSGGGGSDIQMTQSPSSLSASVGDRVTITCKASQNVDTNVAWYQQ |
| | | | KPGQAPKSLIYSASYRYSDVPSRFSGSASGTDFTLTISSVQSEDFATYYCQQYDSYPY |
| | | | TFGGGTKLEIKSGGGGSEVQLVESGGGLVQPGGSLKLSCAASGFTENKYAINWVRQAP |
| | | | GKGLEWVARIRSKYNNYATYYADAVKDRFTISRDDSKNTVYLQMNNLKTEDTAVYYCA |
| | | | RAGNFGSSYISYWAYWGQGTLVTVSSGGGGSGGGGSGGGGSQTVVTQEPSLTVSPGGT |
| | | | VTITCGSSTGAVTSGNYPNWVQKKPGQAPRGLIGGTKFLAPGTPARFSGSLSGGKAAL |
| | | | TLSGVQPEDEAEYYCVLWYSNRWVFGSGTKLTVL |
| 2466 | PM 76-B10x | artificial | QVQLVESGGGLVKPGESLRLSCAASGFTFSDYYMYWVRQAPGKGLEWVAIISDGGYYT |
| | I2K-Full | | YYSDIIKGRFTISRDNAKNSLYLQMNSLKAEDTAVYYCARGFPLLRHGAMDYWGQGTL |
| | Sequence | | VTVSSGGGGSGGGGSGGGGSDIQMTQSPSSLSASVGDRVTITCKASQNVDTNVAWYQQ |
| | | | KPGQAPKSLIYSASYRYSDVPSRFSGSASGTDFTLTISSVQSEDFATYYCQQYDSYPY |
| | | | TFGGGTKLEIKSGGGGSEVQLVESGGGLVQPGGSLKLSCAASGFTFNKYAMNWVRQAP |
| | | | GKGLEWVARIRSKYNNYATYYAEAVKGRFTISRDDSKNTVYLQMNNLKTEDTAVYYCV |
| | | | RNENIGTSYISYWAYWGQGTLVTVSSGGGGSGGGGSGGGGSQTVVTQEPSLTVSPGGT |
| | | | VTITCGSSTGAVTSGNYPNWVQQKPGQAPRGLIGGTKFLAPGTPARFSGSLSGGKAAL |
| | | | TLSGVQPEDEAEYYCVLWYSNRWVFGSGTKLTVL |
| 2467 | PM 76-B10x | artificial | QVQLVESGGGLVKPGESLRLSCAASGFTFSDYYMYWVRQAPGKGLEWVAIISDGGYYT |
| | I2L-Full | | YYSDIIKGRFTISRDNAKNSLYLQMNSLKAEDTAVYYCARGFPLLRHGAMDYWGQGTL |
| | Sequence | | VTVSSGGGGSGGGGSGGGGSDIQMTQSPSSLSASVGDRVTITCKASQNVDTNVAWYQQ |
| | | | KPGQAPKSLIYSASYRYSDVPSRFSGSASGTDFTLTISSVQSEDFATYYCQQYDSYPY |
| | | | TFGGGTKLEIKSGGGGSEVQLVESGGGLVQPGGSLKLSCAASGFTENKYAMNWVRQAP |
| | | | GKGMEWVARIRSKYNNYATYYADAVKDRFTISRDDSKNTLYLQMNNLKTEDTAVYYCV |
| | | | RAGNFGSSYISYFAYWGQGTLVTVSSGGGGSGGGGSGGGGSQTVVTQEPSLTVSPGGT |
| | | | VTITCGSSTGAVTSGNYPNWIQKKPGQAPRGLIGGTKFLAPGTPARFSGSLEGGKAAL |
| | | | TLSGVQPEDEAEYYCVLYYSNRWVFGSGTKLTVL |
| 2468 | PM 76-B10x | artificial | QVQLVESGGGLVKPGESLRLSCAASGFTFSDYYMYWVRQAPGKGLEWVAIISDGGYYT |
| | I2M-Full | | YYSDIIKGRFTISRDNAKNSLYLQMNSLKAEDTAVYYCARGFPLLRHGAMDYWGQGTL |
| | Sequence | | VTVSSGGGGSGGGGSGGGGSDIQMTQSPSSLSASVGDRVTITCKASQNVDTNVAWYQQ |
| | | | KPGQAPKSLIYSASYRYSDVPSRFSGSASGTDFTLTISSVQSEDFATYYCQQYDSYPY |
| | | | TFGGGTKLEIKSGGGGSEVQLVESGGGLVQPGGSLKLSCAASGFTFNKYAMNWVRQAP |
| | | | GKGMEWVARIRSKYNNYATYYADAVKDRFTISRDDSKNTLYLQMNNLKTEDTAVYYCV |
| | | | RAGNFGTSYISYWAYWGQGTLVTVSSGGGGSGGGGSGGGGSQTVVTQEPSLTVSPGGT |
| | | | VTITCGSSTGAVTSGNYPNWVQKKPGQAPRGLIGGTKFLAPGTPARFSGSLSGGKAAL |
| | | | TLSGVQPEDEAEYYCVLWYSNRWVFGSGTKLTVL |
| 2469 | PM 76-B10x | artificial | QVQLVESGGGLVKPGESLRLSCAASGFTFSDYYMYWVRQAPGKGLEWVAIISDGGYYT |
| | I2M2-Full | | YYSDIIKGRFTISRDNAKNSLYLQMNSLKAEDTAVYYCARGFPLLRHGAMDYWGQGTL |
| | Sequence | | VTVSSGGGGSGGGGSGGGGSDIQMTQSPSSLSASVGDRVTITCKASQNVDTNVAWYQQ |
| | | | KPGQAPKSLIYSASYRYSDVPSRFSGSASGTDFTLTISSVQSEDFATYYCQQYDSYPY |
| | | | TFGGGTKLEIKSGGGGSEVQLVESGGGLVQPGGSLKLSCAASGFTENKYAINWVREAP |
| | | | GKGLEWVARIRSKYNNYATYYADAVKDRFTISRDDSKNTAYLQMNNLKTEDTAVYYCV |
| | | | RNANFGTSYISYFAYWGQGTLVTVSSGGGGSGGGGSGGGGSQTVVTQEPSLTVSPGGT |
| | | | VTLTCGSSTGAVTSGNYPNWVQKKPGQAPRGLIGGTKFLAPGTPARFSGSLLGGKAAL |
| | | | TLSGVQPEDEAEYYCVLWYSNRWVFGSGTKLTVL |
| 2470 | PM 76- | artificial | QVQLVESGGGLVKPGESLRLSCAASGFTFSDYYMYWVRQAPGKCLEWVAIISDGGYYT |
| | B10.11 CC x | | YYSDIIKGRFTISRDNAKNSLYLQMNSLKAEDTAVYYCARGFPLLRHGAMDYWGQGTL |
| | I2C x scFc | | VTVSSGGGGSGGGGSGGGGSDIQMTQSPSSLSASVGDRVTITCKASQNVDTNVAWYQQ |
| | -Full | | KPGQAPKSLIYSASYVYWDVPSRFSGSASGTDFTLTISSVQSEDFATYYCQQYDQQLI |
| | Sequence | | TFGCGTKLEIKSGGGGSEVQLVESGGGLVQPGGSLKLSCAASGFTFNKYAMNWVRQAP |
| | | | GKGLEWVARIRSKYNNYATYYADSVKDRFTISRDDSKNTAYLQMNNLKTEDTAVYYCV |
| | | | RHGNFGNSYISYWAYWGQGTLVTVSSGGGGSGGGGSGGGGSQTVVTQEPSLTVSPGGT |
| | | | VTLTCGSSTGAVTSGNYPNWVQQKPGQAPRGLIGGTKFLAPGTPARFSGSLLGGKAAL |
| | | | TLSGVQPEDEAEYYCVLWYSNRWVFGGGTKLTVLGGGGDKTHTCPPCPAPELLGGPSV |
| | | | FLFPPKPKDTLMISRTPEVTCVVVDVSHEDPEVKFNWYVDGVEVHNAKTKPCEEQYGS |
| | | | TYRCVSVLTVLHQDWLNGKEYKCKVSNKALPAPIEKTISKAKGQPREPQVYTLPPSRE |
| | | | EMTKNQVSLTCLVKGFYPSDIAVEWESNGQPENNYKTTPPVLDSDGSFFLYSKLTVDK |
| | | | SRWQQGNVFSCSVMHEALHNHYTQKSLSLSPGKGGGGSGGGGSGGGGSGGGGSGGGGS |
| | | | GGGGSDKTHTCPPCPAPELLGGPSVFLFPPKPKDTLMISRTPEVTCVVVDVSHEDPEV |
| | | | KFNWYVDGVEVHNAKTKPCEEQYGSTYRCVSVLTVLHQDWLNGKEYKCKVSNKALPAP |
| | | | IEKTISKAKGQPREPQVYTLPPSREEMTKNQVSLTCLVKGFYPSDIAVEWESNGQPEN |
| | | | NYKTTPPVLDSDGSFFLYSKLTVDKSRWQQGNVFSCSVMHEALHNHYTQKSLSLSPGK |
| 2471 | PM 76- | artificial | QVQLVESGGGLVKPGESLRLSCAASGFTFSDYYMYWVRQAPGKCLEWVAIISDGGYYT |
| | B10.11 CC x | | YYSDIIKGRFTISRDNAKNSLYLQMNSLKAEDTAVYYCARGFPLLRHGAMDYWGQGTL |
| | I2E x scFc | | VTVSSGGGGSGGGGSGGGGSDIQMTQSPSSLSASVGDRVTITCKASQNVDTNVAWYQQ |
| | -Full | | KPGQAPKSLIYSASYVYWDVPSRFSGSASGTDFTLTISSVQSEDFATYYCQQYDQQLI |
| | Sequence | | TFGCGTKLEIKSGGGGSEVQLVESGGGLVQPGGSLKLSCAASGFTENKYAINWVRQAP |
| | | | GKGLEWVARIRSKYNNYATYYADAVKDRFTISRDDSKNTVYLQMNNLKTEDTAVYYCA |
| | | | RAGNFGSSYISYWAYWGQGTLVTVSSGGGGSGGGGSGGGGSQTVVTQEPSLTVSPGGT |
| | | | VTITCGSSTGAVTSGNYPNWVQKKPGQAPRGLIGGTKFLAPGTPARFSGSLSGGKAAL |
| | | | TLSGVQPEDEAEYYCVLWYSNRWVFGSGTKLTVLGGGGDKTHTCPPCPAPELLGGPSV |
| | | | FLFPPKPKDTLMISRTPEVTCVVVDVSHEDPEVKFNWYVDGVEVHNAKTKPCEEQYGS |
| | | | TYRCVSVLTVLHQDWLNGKEYKCKVSNKALPAPIEKTISKAKGQPREPQVYTLPPSRE |
| | | | EMTKNQVSLTCLVKGFYPSDIAVEWESNGQPENNYKTTPPVLDSDGSFFLYSKLTVDK |
| | | | SRWQQGNVFSCSVMHEALHNHYTQKSLSLSPGKGGGGSGGGGSGGGGSGGGGSGGGGS |
| | | | GGGGSDKTHTCPPCPAPELLGGPSVFLFPPKPKDTLMISRTPEVTCVVVDVSHEDPEV |
| | | | KFNWYVDGVEVHNAKTKPCEEQYGSTYRCVSVLTVLHQDWLNGKEYKCKVSNKALPAP |
| | | | IEKTISKAKGQPREPQVYTLPPSREEMTKNQVSLTCLVKGFYPSDIAVEWESNGQPEN |
| | | | NYKTTPPVLDSDGSFFLYSKLTVDKSRWQQGNVFSCSVMHEALHNHYTQKSLSLSPGK |
| 2472 | PM 76- | artificial | QVQLVESGGGLVKPGESLRLSCAASGFTFSDYYMYWVRQAPGKCLEWVAIISDGGYYT |
| | B10.11 CC x | | YYSDIIKGRFTISRDNAKNSLYLQMNSLKAEDTAVYYCARGFPLLRHGAMDYWGQGTL |
| | I2K x scFc | | VTVSSGGGGSGGGGSGGGGSDIQMTQSPSSLSASVGDRVTITCKASQNVDTNVAWYQQ |
| | -Full | | KPGQAPKSLIYSASYVYWDVPSRFSGSASGTDFTLTISSVQSEDFATYYCQQYDQQLI |
| | Sequence | | TFGCGTKLEIKSGGGGSEVQLVESGGGLVQPGGSLKLSCAASGFTFNKYAMNWVRQAP |
| | | | GKGLEWVARIRSKYNNYATYYAEAVKGRFTISRDDSKNTVYLQMNNLKTEDTAVYYCV |
| | | | RNENIGTSYISYWAYWGQGTLVTVSSGGGGSGGGGSGGGGSQTVVTQEPSLTVSPGGT |
| | | | VTITCGSSTGAVTSGNYPNWVQQKPGQAPRGLIGGTKFLAPGTPARFSGSLSGGKAAL |
| | | | TLSGVQPEDEAEYYCVLWYSNRWVFGSGTKLTVLGGGGDKTHTCPPCPAPELLGGPSV |
| | | | FLFPPKPKDTLMISRTPEVTCVVVDVSHEDPEVKENWYVDGVEVHNAKTKPCEEQYGS |
| | | | TYRCVSVLTVLHQDWLNGKEYKCKVSNKALPAPIEKTISKAKGQPREPQVYTLPPSRE |
| | | | EMTKNQVSLTCLVKGFYPSDIAVEWESNGQPENNYKTTPPVLDSDGSFFLYSKLTVDK |
| | | | SRWQQGNVFSCSVMHEALHNHYTQKSLSLSPGKGGGGSGGGGSGGGGSGGGGSGGGGS |
| | | | GGGGSDKTHTCPPCPAPELLGGPSVFLFPPKPKDTLMISRTPEVTCVVVDVSHEDPEV |
| | | | KFNWYVDGVEVHNAKTKPCEEQYGSTYRCVSVLTVLHQDWLNGKEYKCKVSNKALPAP |
| | | | IEKTISKAKGQPREPQVYTLPPSREEMTKNQVSLTCLVKGFYPSDIAVEWESNGQPEN |
| | | | NYKTTPPVLDSDGSFFLYSKLTVDKSRWQQGNVFSCSVMHEALHNHYTQKSLSLSPGK |
| 2473 | PM 76- | artificial | QVQLVESGGGLVKPGESLRLSCAASGFTFSDYYMYWVRQAPGKCLEWVAIISDGGYYT |
| | B10.11 CC x | | YYSDIIKGRFTISRDNAKNSLYLQMNSLKAEDTAVYYCARGFPLLRHGAMDYWGQGTL |
| | I2L x scFc | | VTVSSGGGGSGGGGSGGGGSDIQMTQSPSSLSASVGDRVTITCKASQNVDTNVAWYQQ |
| | -Full | | KPGQAPKSLIYSASYVYWDVPSRFSGSASGTDFTLTISSVQSEDFATYYCQQYDQQLI |
| | Sequence | | TFGCGTKLEIKSGGGGSEVQLVESGGGLVQPGGSLKLSCAASGFTFNKYAMNWVRQAP |
| | | | GKGMEWVARIRSKYNNYATYYADAVKDRFTISRDDSKNTLYLQMNNLKTEDTAVYYCV |
| | | | RAGNFGSSYISYFAYWGQGTLVTVSSGGGGSGGGGSGGGGSQTVVTQEPSLTVSPGGT |
| | | | VTITCGSSTGAVTSGNYPNWIQKKPGQAPRGLIGGTKFLAPGTPARFSGSLEGGKAAL |
| | | | TLSGVQPEDEAEYYCVLYYSNRWVFGSGTKLTVLGGGGDKTHTCPPCPAPELLGGPSV |
| | | | FLFPPKPKDTLMISRTPEVTCVVVDVSHEDPEVKENWYVDGVEVHNAKTKPCEEQYGS |
| | | | TYRCVSVLTVLHQDWLNGKEYKCKVSNKALPAPIEKTISKAKGQPREPQVYTLPPSRE |
| | | | EMTKNQVSLTCLVKGFYPSDIAVEWESNGQPENNYKTTPPVLDSDGSFFLYSKLTVDK |
| | | | SRWQQGNVFSCSVMHEALHNHYTQKSLSLSPGKGGGGSGGGGSGGGGSGGGGSGGGGS |
| | | | GGGGSDKTHTCPPCPAPELLGGPSVFLFPPKPKDTLMISRTPEVTCVVVDVSHEDPEV |
| | | | KFNWYVDGVEVHNAKTKPCEEQYGSTYRCVSVLTVLHQDWLNGKEYKCKVSNKALPAP |
| | | | IEKTISKAKGQPREPQVYTLPPSREEMTKNQVSLTCLVKGFYPSDIAVEWESNGQPEN |
| | | | NYKTTPPVLDSDGSFFLYSKLTVDKSRWQQGNVFSCSVMHEALHNHYTQKSLSLSPGK |
| 2474 | PM 76- | artificial | QVQLVESGGGLVKPGESLRLSCAASGFTFSDYYMYWVRQAPGKCLEWVAIISDGGYYT |
| | B10.11 CC x | | YYSDIIKGRFTISRDNAKNSLYLQMNSLKAEDTAVYYCARGFPLLRHGAMDYWGQGTL |
| | I2M x scFc | | VTVSSGGGGSGGGGSGGGGSDIQMTQSPSSLSASVGDRVTITCKASQNVDTNVAWYQQ |
| | -Full | | KPGQAPKSLIYSASYVYWDVPSRFSGSASGTDFTLTISSVQSEDFATYYCQQYDQQLI |
| | Sequence | | TFGCGTKLEIKSGGGGSEVQLVESGGGLVQPGGSLKLSCAASGFTENKYAMNWVRQAP |
| | | | GKGMEWVARIRSKYNNYATYYADAVKDRFTISRDDSKNTLYLQMNNLKTEDTAVYYCV |
| | | | RAGNFGTSYISYWAYWGQGTLVTVSSGGGGSGGGGSGGGGSQTVVTQEPSLTVSPGGT |
| | | | VTITCGSSTGAVTSGNYPNWVQKKPGQAPRGLIGGTKFLAPGTPARFSGSLSGGKAAL |
| | | | TLSGVQPEDEAEYYCVLWYSNRWVFGSGTKLTVLGGGGDKTHTCPPCPAPELLGGPSV |
| | | | FLFPPKPKDTLMISRTPEVTCVVVDVSHEDPEVKFNWYVDGVEVHNAKTKPCEEQYGS |
| | | | TYRCVSVLTVLHQDWLNGKEYKCKVSNKALPAPIEKTISKAKGQPREPQVYTLPPSRE |
| | | | EMTKNQVSLTCLVKGFYPSDIAVEWESNGQPENNYKTTPPVLDSDGSFFLYSKLTVDK |
| | | | SRWQQGNVFSCSVMHEALHNHYTQKSLSLSPGKGGGGSGGGGSGGGGSGGGGSGGGGS |
| | | | GGGGSDKTHTCPPCPAPELLGGPSVFLFPPKPKDTLMISRTPEVTCVVVDVSHEDPEV |
| | | | KFNWYVDGVEVHNAKTKPCEEQYGSTYRCVSVLTVLHQDWLNGKEYKCKVSNKALPAP |
| | | | IEKTISKAKGQPREPQVYTLPPSREEMTKNQVSLTCLVKGFYPSDIAVEWESNGQPEN |
| | | | NYKTTPPVLDSDGSFFLYSKLTVDKSRWQQGNVFSCSVMHEALHNHYTQKSLSLSPGK |
| 2475 | PM 76- | artificial | QVQLVESGGGLVKPGESLRLSCAASGFTFSDYYMYWVRQAPGKCLEWVAIISDGGYYT |
| | B10.11 CC x | | YYSDIIKGRFTISRDNAKNSLYLQMNSLKAEDTAVYYCARGFPLLRHGAMDYWGQGTL |
| | I2M2 x scFc | | VTVSSGGGGSGGGGSGGGGSDIQMTQSPSSLSASVGDRVTITCKASQNVDTNVAWYQQ |
| | -Full | | KPGQAPKSLIYSASYVYWDVPSRFSGSASGTDFTLTISSVQSEDFATYYCQQYDQQLI |
| | Sequence | | TFGCGTKLEIKSGGGGSEVQLVESGGGLVQPGGSLKLSCAASGFTENKYAINWVREAP |
| | | | GKGLEWVARIRSKYNNYATYYADAVKDRFTISRDDSKNTAYLQMNNLKTEDTAVYYCV |
| | | | RNANFGTSYISYFAYWGQGTLVTVSSGGGGSGGGGSGGGGSQTVVTQEPSLTVSPGGT |
| | | | VTLTCGSSTGAVTSGNYPNWVQKKPGQAPRGLIGGTKFLAPGTPARFSGSLLGGKAAL |
| | | | TLSGVQPEDEAEYYCVLWYSNRWVFGSGTKLTVLGGGGDKTHTCPPCPAPELLGGPSV |
| | | | FLFPPKPKDTLMISRTPEVTCVVVDVSHEDPEVKENWYVDGVEVHNAKTKPCEEQYGS |
| | | | TYRCVSVLTVLHQDWLNGKEYKCKVSNKALPAPIEKTISKAKGQPREPQVYTLPPSRE |
| | | | EMTKNQVSLTCLVKGFYPSDIAVEWESNGQPENNYKTTPPVLDSDGSFFLYSKLTVDK |
| | | | SRWQQGNVFSCSVMHEALHNHYTQKSLSLSPGKGGGGSGGGGSGGGGSGGGGSGGGGS |
| | | | GGGGSDKTHTCPPCPAPELLGGPSVFLFPPKPKDTLMISRTPEVTCVVVDVSHEDPEV |
| | | | KFNWYVDGVEVHNAKTKPCEEQYGSTYRCVSVLTVLHQDWLNGKEYKCKVSNKALPAP |
| | | | IEKTISKAKGQPREPQVYTLPPSREEMTKNQVSLTCLVKGFYPSDIAVEWESNGQPEN |
| | | | NYKTTPPVLDSDGSFFLYSKLTVDKSRWQQGNVFSCSVMHEALHNHYTQKSLSLSPGK |
| 2505 | CD20 99-E5 | artificial | QVQLVQSGAEVKKPGASVKVSCKASGYTFTSYWMHWVRQAPGQCLEWIGYITPSTGYT |
| | CC x CD22 | | EYNQKFKGRVTMTRDKSTSTVYMELSSLTSEDTAVYYCARVHDYDRAMEYWGQGTTVT |
| | 28-B7 N65S | | VSSGGGGSGGGGSGGGGSDIQMTQSPSSLSASVGDRVTITCKASQDINKYIAWYQQKP |
| | CC x I2C x | | GKGPKLLIYYTSTLQPGVPSRFSGSGSGTDFTFTISSLQPEDIATYYCLQYASYPFTF |
| | scFc-Full | | GCGTRLEIKSGGGGSQVQLVQSGAEVKKPGASVKVSCKASGYTFTSYGISWVRQAPGQ |
| | Sequence | | CLEWMGWISAYSGNAIYAQKLQGRVTMTRDTSTSTAYMELRSLRSDDTAVYYCARDPD |
| | | | YYGSGSYSDYWGQGTLVTVSSGGGGSGGGGSGGGGSEIVLTQSPATLSVSPGERATLS |
| | | | CRASQSVSSNLAWYQQKPGQAPRLLIYGASSRATGIPARFSGSGSGTEFTLTISSLQS |
| | | | EDFAVYYCQQYHSWPLLTFGCGTKVEIKSGGGGSEVQLVESGGGLVQPGGSLKLSCAA |
| | | | SGFTFNKYAMNWVRQAPGKGLEWVARIRSKYNNYATYYADSVKDRFTISRDDSKNTAY |
| | | | LQMNNLKTEDTAVYYCVRHGNFGNSYISYWAYWGQGTLVTVSSGGGGSGGGGSGGGGS |
| | | | QTVVTQEPSLTVSPGGTVTLTCGSSTGAVTSGNYPNWVQQKPGQAPRGLIGGTKFLAP |
| | | | GTPARFSGSLLGGKAALTLSGVQPEDEAEYYCVLWYSNRWVFGGGTKLTVLGGGGDKT |
| | | | HTCPPCPAPELLGGPSVFLFPPKPKDTLMISRTPEVTCVVVDVSHEDPEVKFNWYVDG |
| | | | VEVHNAKTKPCEEQYGSTYRCVSVLTVLHQDWLNGKEYKCKVSNKALPAPIEKTISKA |
| | | | KGQPREPQVYTLPPSREEMTKNQVSLTCLVKGFYPSDIAVEWESNGQPENNYKTTPPV |
| | | | LDSDGSFFLYSKLTVDKSRWQQGNVFSCSVMHEALHNHYTQKSLSLSPGKGGGGSGGG |
| | | | GSGGGGSGGGGSGGGGSGGGGSDKTHTCPPCPAPELLGGPSVFLFPPKPKDTLMISRT |
| | | | PEVTCVVVDVSHEDPEVKFNWYVDGVEVHNAKTKPCEEQYGSTYRCVSVLTVLHQDWL |
| | | | NGKEYKCKVSNKALPAPIEKTISKAKGQPREPQVYTLPPSREEMTKNQVSLTCLVKGF |
| | | | YPSDIAVEWESNGQPENNYKTTPPVLDSDGSFFLYSKLTVDKSRWQQGNVFSCSVMHE |
| | | | ALHNHYTQKSLSLSPGK |
| 2506 | CD20 99-E5 | artificial | QVQLVQSGAEVKKPGASVKVSCKASGYTFTSYWMHWVRQAPGQCLEWIGYITPSTGYT |
| | CC x CD22 | | EYNQKFKGRVTMTRDKSTSTVYMELSSLTSEDTAVYYCARVHDYDRAMEYWGQGTTVT |
| | 28-B7 N65S | | VSSGGGGSGGGGSGGGGSDIQMTQSPSSLSASVGDRVTITCKASQDINKYIAWYQQKP |
| | CC x I2E x | | GKGPKLLIYYTSTLQPGVPSRFSGSGSGTDFTFTISSLQPEDIATYYCLQYASYPFTF |
| | scFc-Full | | GCGTRLEIKSGGGGSQVQLVQSGAEVKKPGASVKVSCKASGYTFTSYGISWVRQAPGQ |
| | Sequence | | CLEWMGWISAYSGNAIYAQKLQGRVTMTRDTSTSTAYMELRSLRSDDTAVYYCARDPD |
| | | | YYGSGSYSDYWGQGTLVTVSSGGGGSGGGGSGGGGSEIVLTQSPATLSVSPGERATLS |
| | | | CRASQSVSSNLAWYQQKPGQAPRLLIYGASSRATGIPARFSGSGSGTEFTLTISSLQS |
| | | | EDFAVYYCQQYHSWPLLTFGCGTKVEIKSGGGGSEVQLVESGGGLVQPGGSLKLSCAA |
| | | | SGFTFNKYAINWVRQAPGKGLEWVARIRSKYNNYATYYADAVKDRFTISRDDSKNTVY |
| | | | LQMNNLKTEDTAVYYCARAGNFGSSYISYWAYWGQGTLVTVSSGGGGSGGGGSGGGGS |
| | | | QTVVTQEPSLTVSPGGTVTITCGSSTGAVTSGNYPNWVQKKPGQAPRGLIGGTKFLAP |
| | | | GTPARFSGSLSGGKAALTLSGVQPEDEAEYYCVLWYSNRWVFGSGTKLTVLGGGGDKT |
| | | | HTCPPCPAPELLGGPSVFLFPPKPKDTLMISRTPEVTCVVVDVSHEDPEVKENWYVDG |
| | | | VEVHNAKTKPCEEQYGSTYRCVSVLTVLHQDWLNGKEYKCKVSNKALPAPIEKTISKA |
| | | | KGQPREPQVYTLPPSREEMTKNQVSLTCLVKGFYPSDIAVEWESNGQPENNYKTTPPV |
| | | | LDSDGSFFLYSKLTVDKSRWQQGNVFSCSVMHEALHNHYTQKSLSLSPGKGGGGSGGG |
| | | | GSGGGGSGGGGSGGGGSGGGGSDKTHTCPPCPAPELLGGPSVFLFPPKPKDTLMISRT |
| | | | PEVTCVVVDVSHEDPEVKFNWYVDGVEVHNAKTKPCEEQYGSTYRCVSVLTVLHQDWL |
| | | | NGKEYKCKVSNKALPAPIEKTISKAKGQPREPQVYTLPPSREEMTKNQVSLTCLVKGF |
| | | | YPSDIAVEWESNGQPENNYKTTPPVLDSDGSFFLYSKLTVDKSRWQQGNVFSCSVMHE |
| | | | ALHNHYTQKSLSLSPGK |
| | | | QVQLVQSGAEVKKPGASVKVSCKASGYTFTSYWMHWVRQAPGQCLEWIGYITPSTGYT |
| 2507 | CD20 99-E5 | artificial | EYNQKFKGRVTMTRDKSTSTVYMELSSLTSEDTAVYYCARVHDYDRAMEYWGQGTTVT |
| | CC x CD22 | | VSSGGGGSGGGGSGGGGSDIQMTQSPSSLSASVGDRVTITCKASQDINKYIAWYQQKP |
| | 28-B7 N65S | | GKGPKLLIYYTSTLQPGVPSRFSGSGSGTDFTFTISSLQPEDIATYYCLQYASYPFTF |
| | CC x I2K x | | GCGTRLEIKSGGGGSQVQLVQSGAEVKKPGASVKVSCKASGYTFTSYGISWVRQAPGQ |
| | scFc-Full | | CLEWMGWISAYSGNAIYAQKLQGRVTMTRDTSTSTAYMELRSLRSDDTAVYYCARDPD |
| | Sequence | | YYGSGSYSDYWGQGTLVTVSSGGGGSGGGGSGGGGSEIVLTQSPATLSVSPGERATLS |
| | | | CRASQSVSSNLAWYQQKPGQAPRLLIYGASSRATGIPARFSGSGSGTEFTLTISSLQS |
| | | | EDFAVYYCQQYHSWPLLTFGCGTKVEIKSGGGGSEVQLVESGGGLVQPGGSLKLSCAA |
| | | | SGFTFNKYAMNWVRQAPGKGLEWVARIRSKYNNYATYYAEAVKGRFTISRDDSKNTVY |
| | | | LQMNNLKTEDTAVYYCVRNENIGTSYISYWAYWGQGTLVTVSSGGGGSGGGGSGGGGS |
| | | | QTVVTQEPSLTVSPGGTVTITCGSSTGAVTSGNYPNWVQQKPGQAPRGLIGGTKFLAP |
| | | | GTPARFSGSLSGGKAALTLSGVQPEDEAEYYCVLWYSNRWVFGSGTKLTVLGGGGDKT |
| | | | HTCPPCPAPELLGGPSVFLFPPKPKDTLMISRTPEVTCVVVDVSHEDPEVKFNWYVDG |
| | | | VEVHNAKTKPCEEQYGSTYRCVSVLTVLHQDWLNGKEYKCKVSNKALPAPIEKTISKA |
| | | | KGQPREPQVYTLPPSREEMTKNQVSLTCLVKGFYPSDIAVEWESNGQPENNYKTTPPV |
| | | | LDSDGSFFLYSKLTVDKSRWQQGNVFSCSVMHEALHNHYTQKSLSLSPGKGGGGSGGG |
| | | | GSGGGGSGGGGSGGGGSGGGGSDKTHTCPPCPAPELLGGPSVFLFPPKPKDTLMISRT |
| | | | PEVTCVVVDVSHEDPEVKFNWYVDGVEVHNAKTKPCEEQYGSTYRCVSVLTVLHQDWL |
| | | | NGKEYKCKVSNKALPAPIEKTISKAKGQPREPQVYTLPPSREEMTKNQVSLTCLVKGF |
| | | | YPSDIAVEWESNGQPENNYKTTPPVLDSDGSFFLYSKLTVDKSRWQQGNVFSCSVMHE |
| | | | ALHNHYTQKSLSLSPGK |
| | | | QVQLVQSGAEVKKPGASVKVSCKASGYTFTSYWMHWVRQAPGQCLEWIGYITPSTGYT |
| 2508 | CD20 99-E5 | artificial | EYNQKFKGRVTMTRDKSTSTVYMELSSLTSEDTAVYYCARVHDYDRAMEYWGQGTTVT |
| | CC x CD22 | | VSSGGGGSGGGGSGGGGSDIQMTQSPSSLSASVGDRVTITCKASQDINKYIAWYQQKP |
| | 28-B7 N65S | | GKGPKLLIYYTSTLQPGVPSRFSGSGSGTDFTFTISSLQPEDIATYYCLQYASYPFTF |
| | CC x I2L x | | GCGTRLEIKSGGGGSQVQLVQSGAEVKKPGASVKVSCKASGYTFTSYGISWVRQAPGQ |
| | scFc-Full | | CLEWMGWISAYSGNAIYAQKLQGRVTMTRDTSTSTAYMELRSLRSDDTAVYYCARDPD |
| | Sequence | | YYGSGSYSDYWGQGTLVTVSSGGGGSGGGGSGGGGSEIVLTQSPATLSVSPGERATLS |
| | | | CRASQSVSSNLAWYQQKPGQAPRLLIYGASSRATGIPARFSGSGSGTEFTLTISSLQS |
| | | | EDFAVYYCQQYHSWPLLTFGCGTKVEIKSGGGGSEVQLVESGGGLVQPGGSLKLSCAA |
| | | | SGFTFNKYAMNWVRQAPGKGMEWVARIRSKYNNYATYYADAVKDRFTISRDDSKNTLY |
| | | | LQMNNLKTEDTAVYYCVRAGNFGSSYISYFAYWGQGTLVTVSSGGGGSGGGGSGGGGS |
| | | | QTVVTQEPSLTVSPGGTVTITCGSSTGAVTSGNYPNWIQKKPGQAPRGLIGGTKFLAP |
| | | | GTPARFSGSLEGGKAALTLSGVQPEDEAEYYCVLYYSNRWVFGSGTKLTVLGGGGDKT |
| | | | HTCPPCPAPELLGGPSVFLFPPKPKDTLMISRTPEVTCVVVDVSHEDPEVKENWYVDG |
| | | | VEVHNAKTKPCEEQYGSTYRCVSVLTVLHQDWLNGKEYKCKVSNKALPAPIEKTISKA |
| | | | KGQPREPQVYTLPPSREEMTKNQVSLTCLVKGFYPSDIAVEWESNGQPENNYKTTPPV |
| | | | LDSDGSFFLYSKLTVDKSRWQQGNVFSCSVMHEALHNHYTQKSLSLSPGKGGGGSGGG |
| | | | GSGGGGSGGGGSGGGGSGGGGSDKTHTCPPCPAPELLGGPSVFLFPPKPKDTLMISRT |
| | | | PEVTCVVVDVSHEDPEVKFNWYVDGVEVHNAKTKPCEEQYGSTYRCVSVLTVLHQDWL |
| | | | NGKEYKCKVSNKALPAPIEKTISKAKGQPREPQVYTLPPSREEMTKNQVSLTCLVKGF |
| | | | YPSDIAVEWESNGQPENNYKTTPPVLDSDGSFFLYSKLTVDKSRWQQGNVFSCSVMHE |
| | | | ALHNHYTQKSLSLSPGK |
| 2509 | CD20 99-E5 | artificial | QVQLVQSGAEVKKPGASVKVSCKASGYTFTSYWMHWVRQAPGQCLEWIGYITPSTGYT |
| | CC x CD22 | | EYNQKFKGRVTMTRDKSTSTVYMELSSLTSEDTAVYYCARVHDYDRAMEYWGQGTTVT |
| | 28-B7 N65S | | VSSGGGGSGGGGSGGGGSDIQMTQSPSSLSASVGDRVTITCKASQDINKYIAWYQQKP |
| | CC x I2M x | | GKGPKLLIYYTSTLQPGVPSRFSGSGSGTDFTFTISSLQPEDIATYYCLQYASYPFTF |
| | scFc-Full | | GCGTRLEIKSGGGGSQVQLVQSGAEVKKPGASVKVSCKASGYTFTSYGISWVRQAPGQ |
| | Sequence | | CLEWMGWISAYSGNAIYAQKLQGRVTMTRDTSTSTAYMELRSLRSDDTAVYYCARDPD |
| | | | YYGSGSYSDYWGQGTLVTVSSGGGGSGGGGSGGGGSEIVLTQSPATLSVSPGERATLS |
| | | | CRASQSVSSNLAWYQQKPGQAPRLLIYGASSRATGIPARFSGSGSGTEFTLTISSLQS |
| | | | EDFAVYYCQQYHSWPLLTFGCGTKVEIKSGGGGSEVQLVESGGGLVQPGGSLKLSCAA |
| | | | SGFTFNKYAMNWVRQAPGKGMEWVARIRSKYNNYATYYADAVKDRFTISRDDSKNTLY |
| | | | LQMNNLKTEDTAVYYCVRAGNFGTSYISYWAYWGQGTLVTVSSGGGGSGGGGSGGGGS |
| | | | QTVVTQEPSLTVSPGGTVTITCGSSTGAVTSGNYPNWVQKKPGQAPRGLIGGTKFLAP |
| | | | GTPARFSGSLSGGKAALTLSGVQPEDEAEYYCVLWYSNRWVFGSGTKLTVLGGGGDKT |
| | | | HTCPPCPAPELLGGPSVFLFPPKPKDTLMISRTPEVTCVVVDVSHEDPEVKENWYVDG |
| | | | VEVHNAKTKPCEEQYGSTYRCVSVLTVLHQDWLNGKEYKCKVSNKALPAPIEKTISKA |
| | | | KGQPREPQVYTLPPSREEMTKNQVSLTCLVKGFYPSDIAVEWESNGQPENNYKTTPPV |
| | | | LDSDGSFFLYSKLTVDKSRWQQGNVFSCSVMHEALHNHYTQKSLSLSPGKGGGGSGGG |
| | | | GSGGGGSGGGGSGGGGSGGGGSDKTHTCPPCPAPELLGGPSVFLFPPKPKDTLMISRT |
| | | | PEVTCVVVDVSHEDPEVKFNWYVDGVEVHNAKTKPCEEQYGSTYRCVSVLTVLHQDWL |
| | | | NGKEYKCKVSNKALPAPIEKTISKAKGQPREPQVYTLPPSREEMTKNQVSLTCLVKGF |
| | | | YPSDIAVEWESNGQPENNYKTTPPVLDSDGSFFLYSKLTVDKSRWQQGNVFSCSVMHE |
| | | | ALHNHYTQKSLSLSPGK |
| 2510 | CD20 99-E5 | artificial | QVQLVQSGAEVKKPGASVKVSCKASGYTFTSYWMHWVRQAPGQCLEWIGYITPSTGYT |
| | CC x CD22 | | EYNQKFKGRVTMTRDKSTSTVYMELSSLTSEDTAVYYCARVHDYDRAMEYWGQGTTVT |
| | 28-B7 N65S | | VSSGGGGSGGGGSGGGGSDIQMTQSPSSLSASVGDRVTITCKASQDINKYIAWYQQKP |
| | CC x I2M2 x | | GKGPKLLIYYTSTLQPGVPSRFSGSGSGTDFTFTISSLQPEDIATYYCLQYASYPFTF |
| | scFc-Full | | GCGTRLEIKSGGGGSQVQLVQSGAEVKKPGASVKVSCKASGYTFTSYGISWVRQAPGQ |
| | Sequence | | CLEWMGWISAYSGNAIYAQKLQGRVTMTRDTSTSTAYMELRSLRSDDTAVYYCARDPD |
| | | | YYGSGSYSDYWGQGTLVTVSSGGGGSGGGGSGGGGSEIVLTQSPATLSVSPGERATLS |
| | | | CRASQSVSSNLAWYQQKPGQAPRLLIYGASSRATGIPARFSGSGSGTEFTLTISSLQS |
| | | | EDFAVYYCQQYHSWPLLTFGCGTKVEIKSGGGGSEVQLVESGGGLVQPGGSLKLSCAA |
| | | | SGFTFNKYAINWVREAPGKGLEWVARIRSKYNNYATYYADAVKDRFTISRDDSKNTAY |
| | | | LQMNNLKTEDTAVYYCVRNANFGTSYISYFAYWGQGTLVTVSSGGGGSGGGGSGGGGS |
| | | | QTVVTQEPSLTVSPGGTVTLTCGSSTGAVTSGNYPNWVQKKPGQAPRGLIGGTKFLAP |
| | | | GTPARFSGSLLGGKAALTLSGVQPEDEAEYYCVLWYSNRWVFGSGTKLTVLGGGGDKT |
| | | | HTCPPCPAPELLGGPSVFLFPPKPKDTLMISRTPEVTCVVVDVSHEDPEVKENWYVDG |
| | | | VEVHNAKTKPCEEQYGSTYRCVSVLTVLHQDWLNGKEYKCKVSNKALPAPIEKTISKA |
| | | | KGQPREPQVYTLPPSREEMTKNQVSLTCLVKGFYPSDIAVEWESNGQPENNYKTTPPV |
| | | | LDSDGSFFLYSKLTVDKSRWQQGNVFSCSVMHEALHNHYTQKSLSLSPGKGGGGSGGG |
| | | | GSGGGGSGGGGSGGGGSGGGGSDKTHTCPPCPAPELLGGPSVFLFPPKPKDTLMISRT |
| | | | PEVTCVVVDVSHEDPEVKFNWYVDGVEVHNAKTKPCEEQYGSTYRCVSVLTVLHQDWL |
| | | | NGKEYKCKVSNKALPAPIEKTISKAKGQPREPQVYTLPPSREEMTKNQVSLTCLVKGF |
| | | | YPSDIAVEWESNGQPENNYKTTPPVLDSDGSFFLYSKLTVDKSRWQQGNVFSCSVMHE |
| | | | ALHNHYTQKSLSLSPGK |
| 2511 | CD22 28- | artificial | QVQLVQSGAEVKKPGASVKVSCKASGYTFTSYGISWVRQAPGQCLEWMGWISAYSGNA |
| | B7N65S CC x | | IYAQKLQGRVTMTRDTSTSTAYMELRSLRSDDTAVYYCARDPDYYGSGSYSDYWGQGT |
| | I2C x | | LVTVSSGGGGSGGGGSGGGGSEIVLTQSPATLSVSPGERATLSCRASQSVSSNLAWYQ |
| | (G4S) 3 x | | QKPGQAPRLLIYGASSRATGIPARFSGSGSGTEFTLTISSLQSEDFAVYYCQQYHSWP |
| | scFc x | | LLTFGCGTKVEIKSGGGGSEVQLVESGGGLVQPGGSLKLSCAASGFTENKYAMNWVRQ |
| | (G4S) 3 x | | APGKGLEWVARIRSKYNNYATYYADSVKDRFTISRDDSKNTAYLQMNNLKTEDTAVYY |
| | CD20 99E5 | | CVRHGNFGNSYISYWAYWGQGTLVTVSSGGGGSGGGGSGGGGSQTVVTQEPSLTVSPG |
| | CC x I2C- | | GTVTLTCGSSTGAVTSGNYPNWVQQKPGQAPRGLIGGTKFLAPGTPARFSGSLLGGKA |
| | Full | | ALTLSGVQPEDEAEYYCVLWYSNRWVFGGGTKLTVLGGGGSGGGGSGGGGSDKTHTCP |
| | Sequence | | PCPAPELLGGPSVFLFPPKPKDTLMISRTPEVTCVVVDVSHEDPEVKFNWYVDGVEVH |
| | | | NAKTKPCEEQYGSTYRCVSVLTVLHQDWLNGKEYKCKVSNKALPAPIEKTISKAKGQP |
| | | | REPQVYTLPPSREEMTKNQVSLTCLVKGFYPSDIAVEWESNGQPENNYKTTPPVLDSD |
| | | | GSFFLYSKLTVDKSRWQQGNVFSCSVMHEALHNHYTQKSLSLSPGKGGGGSGGGGSGG |
| | | | GGSGGGGSGGGGSGGGGSDKTHTCPPCPAPELLGGPSVFLFPPKPKDTLMISRTPEVT |
| | | | CVVVDVSHEDPEVKFNWYVDGVEVHNAKTKPCEEQYGSTYRCVSVLTVLHQDWLNGKE |
| | | | YKCKVSNKALPAPIEKTISKAKGQPREPQVYTLPPSREEMTKNQVSLTCLVKGFYPSD |
| | | | IAVEWESNGQPENNYKTTPPVLDSDGSFFLYSKLTVDKSRWQQGNVFSCSVMHEALHN |
| | | | HYTQKSLSLSPGKGGGGSGGGGSGGGGSQVQLVQSGAEVKKPGASVKVSCKASGYTFT |
| | | | SYWMHWVRQAPGQCLEWIGYITPSTGYTEYNQKFKGRVTMTRDKSTSTVYMELSSLTS |
| | | | EDTAVYYCARVHDYDRAMEYWGQGTTVTVSSGGGGSGGGGSGGGGSDIQMTQSPSSLS |
| | | | ASVGDRVTITCKASQDINKYIAWYQQKPGKGPKLLIYYTSTLQPGVPSRFSGSGSGTD |
| | | | FTFTISSLQPEDIATYYCLQYASYPFTFGCGTRLEIKSGGGGSEVQLVESGGGLVQPG |
| | | | GSLKLSCAASGFTFNKYAMNWVRQAPGKGLEWVARIRSKYNNYATYYADSVKDRFTIS |
| | | | RDDSKNTAYLQMNNLKTEDTAVYYCVRHGNFGNSYISYWAYWGQGTLVTVSSGGGGSG |
| | | | GGGSGGGGSQTVVTQEPSLTVSPGGTVTLTCGSSTGAVTSGNYPNWVQQKPGQAPRGL |
| | | | IGGTKFLAPGTPARFSGSLLGGKAALTLSGVQPEDEAEYYCVLWYSNRWVFGGGTKLT |
| | | | VL |
| 2512 | CD22 28- | artificial | QVQLVQSGAEVKKPGASVKVSCKASGYTFTSYGISWVRQAPGQCLEWMGWISAYSGNA |
| | B7N65S CC x | | IYAQKLQGRVTMTRDTSTSTAYMELRSLRSDDTAVYYCARDPDYYGSGSYSDYWGQGT |
| | I2E x | | LVTVSSGGGGSGGGGSGGGGSEIVLTQSPATLSVSPGERATLSCRASQSVSSNLAWYQ |
| | (G4S ) 3 x | | QKPGQAPRLLIYGASSRATGIPARFSGSGSGTEFTLTISSLQSEDFAVYYCQQYHSWP |
| | ScFc x | | LLTFGCGTKVEIKSGGGGSEVQLVESGGGLVQPGGSLKLSCAASGFTFNKYAINWVRQ |
| | (G4S) 3 x | | APGKGLEWVARIRSKYNNYATYYADAVKDRFTISRDDSKNTVYLQMNNLKTEDTAVYY |
| | CD20 99E5 | | CARAGNFGSSYISYWAYWGQGTLVTVSSGGGGSGGGGSGGGGSQTVVTQEPSLTVSPG |
| | CC x I2E- | | GTVTITCGSSTGAVTSGNYPNWVQKKPGQAPRGLIGGTKFLAPGTPARFSGSLSGGKA |
| | Full | | ALTLSGVQPEDEAEYYCVLWYSNRWVFGSGTKLTVLGGGGSGGGGSGGGGSDKTHTCP |
| | Sequence | | PCPAPELLGGPSVFLFPPKPKDTLMISRTPEVTCVVVDVSHEDPEVKENWYVDGVEVH |
| | | | NAKTKPCEEQYGSTYRCVSVLTVLHQDWLNGKEYKCKVSNKALPAPIEKTISKAKGQP |
| | | | REPQVYTLPPSREEMTKNQVSLTCLVKGFYPSDIAVEWESNGQPENNYKTTPPVLDSD |
| | | | GSFFLYSKLTVDKSRWQQGNVFSCSVMHEALHNHYTQKSLSLSPGKGGGGSGGGGSGG |
| | | | GGSGGGGSGGGGSGGGGSDKTHTCPPCPAPELLGGPSVFLFPPKPKDTLMISRTPEVT |
| | | | CVVVDVSHEDPEVKFNWYVDGVEVHNAKTKPCEEQYGSTYRCVSVLTVLHQDWLNGKE |
| | | | YKCKVSNKALPAPIEKTISKAKGQPREPQVYTLPPSREEMTKNQVSLTCLVKGFYPSD |
| | | | IAVEWESNGQPENNYKTTPPVLDSDGSFFLYSKLTVDKSRWQQGNVFSCSVMHEALHN |
| | | | HYTQKSLSLSPGKGGGGSGGGGSGGGGSQVQLVQSGAEVKKPGASVKVSCKASGYTFT |
| | | | SYWMHWVRQAPGQCLEWIGYITPSTGYTEYNQKFKGRVTMTRDKSTSTVYMELSSLTS |
| | | | EDTAVYYCARVHDYDRAMEYWGQGTTVTVSSGGGGSGGGGSGGGGSDIQMTQSPSSLS |
| | | | ASVGDRVTITCKASQDINKYIAWYQQKPGKGPKLLIYYTSTLQPGVPSRFSGSGSGTD |
| | | | FTFTISSLQPEDIATYYCLQYASYPFTFGCGTRLEIKSGGGGSEVQLVESGGGLVQPG |
| | | | GSLKLSCAASGFTFNKYAINWVRQAPGKGLEWVARIRSKYNNYATYYADAVKDRFTIS |
| | | | RDDSKNTVYLQMNNLKTEDTAVYYCARAGNFGSSYISYWAYWGQGTLVTVSSGGGGSG |
| | | | GGGSGGGGSQTVVTQEPSLTVSPGGTVTITCGSSTGAVTSGNYPNWVQKKPGQAPRGL |
| | | | IGGTKFLAPGTPARFSGSLSGGKAALTLSGVQPEDEAEYYCVLWYSNRWVFGSGTKLT |
| | | | VL |
| 2513 | CD22 28- | artificial | QVQLVQSGAEVKKPGASVKVSCKASGYTFTSYGISWVRQAPGQCLEWMGWISAYSGNA |
| | B7N65S CC x | | IYAQKLQGRVTMTRDTSTSTAYMELRSLRSDDTAVYYCARDPDYYGSGSYSDYWGQGT |
| | I2K x | | LVTVSSGGGGSGGGGSGGGGSEIVLTQSPATLSVSPGERATLSCRASQSVSSNLAWYQ |
| | (G4S) 3 x | | QKPGQAPRLLIYGASSRATGIPARFSGSGSGTEFTLTISSLQSEDFAVYYCQQYHSWP |
| | ScFc x | | LLTFGCGTKVEIKSGGGGSEVQLVESGGGLVQPGGSLKLSCAASGFTFNKYAMNWVRQ |
| | (G4S) 3 x | | APGKGLEWVARIRSKYNNYATYYAEAVKGRFTISRDDSKNTVYLQMNNLKTEDTAVYY |
| | CD20 99E5 | | CVRNENIGTSYISYWAYWGQGTLVTVSSGGGGSGGGGSGGGGSQTVVTQEPSLTVSPG |
| | CC x I2K- | | GTVTITCGSSTGAVTSGNYPNWVQQKPGQAPRGLIGGTKFLAPGTPARFSGSLSGGKA |
| | Full | | ALTLSGVQPEDEAEYYCVLWYSNRWVFGSGTKLTVLGGGGSGGGGSGGGGSDKTHTCP |
| | Sequence | | PCPAPELLGGPSVFLFPPKPKDTLMISRTPEVTCVVVDVSHEDPEVKFNWYVDGVEVH |
| | | | NAKTKPCEEQYGSTYRCVSVLTVLHQDWLNGKEYKCKVSNKALPAPIEKTISKAKGQP |
| | | | REPQVYTLPPSREEMTKNQVSLTCLVKGFYPSDIAVEWESNGQPENNYKTTPPVLDSD |
| | | | GSFFLYSKLTVDKSRWQQGNVFSCSVMHEALHNHYTQKSLSLSPGKGGGGSGGGGSGG |
| | | | GGSGGGGSGGGGSGGGGSDKTHTCPPCPAPELLGGPSVFLFPPKPKDTLMISRTPEVT |
| | | | CVVVDVSHEDPEVKFNWYVDGVEVHNAKTKPCEEQYGSTYRCVSVLTVLHQDWLNGKE |
| | | | YKCKVSNKALPAPIEKTISKAKGQPREPQVYTLPPSREEMTKNQVSLTCLVKGFYPSD |
| | | | IAVEWESNGQPENNYKTTPPVLDSDGSFFLYSKLTVDKSRWQQGNVFSCSVMHEALHN |
| | | | HYTQKSLSLSPGKGGGGSGGGGSGGGGSQVQLVQSGAEVKKPGASVKVSCKASGYTFT |
| | | | SYWMHWVRQAPGQCLEWIGYITPSTGYTEYNQKFKGRVTMTRDKSTSTVYMELSSLTS |
| | | | EDTAVYYCARVHDYDRAMEYWGQGTTVTVSSGGGGSGGGGSGGGGSDIQMTQSPSSLS |
| | | | ASVGDRVTITCKASQDINKYIAWYQQKPGKGPKLLIYYTSTLQPGVPSRFSGSGSGTD |
| | | | FTFTISSLQPEDIATYYCLQYASYPFTFGCGTRLEIKSGGGGSEVQLVESGGGLVQPG |
| | | | GSLKLSCAASGFTFNKYAMNWVRQAPGKGLEWVARIRSKYNNYATYYAEAVKGRFTIS |
| | | | RDDSKNTVYLQMNNLKTEDTAVYYCVRNENIGTSYISYWAYWGQGTLVTVSSGGGGSG |
| | | | GGGSGGGGSQTVVTQEPSLTVSPGGTVTITCGSSTGAVTSGNYPNWVQQKPGQAPRGL |
| | | | IGGTKFLAPGTPARFSGSLSGGKAALTLSGVQPEDEAEYYCVLWYSNRWVFGSGTKLT |
| | | | VL |
| 2514 | CD22 28- | artificial | QVQLVQSGAEVKKPGASVKVSCKASGYTFTSYGISWVRQAPGQCLEWMGWISAYSGNA |
| | B7N65S CC x | | IYAQKLQGRVTMTRDTSTSTAYMELRSLRSDDTAVYYCARDPDYYGSGSYSDYWGQGT |
| | I2L x | | LVTVSSGGGGSGGGGSGGGGSEIVLTQSPATLSVSPGERATLSCRASQSVSSNLAWYQ |
| | (G4S) 3 x | | QKPGQAPRLLIYGASSRATGIPARFSGSGSGTEFTLTISSLQSEDFAVYYCQQYHSWP |
| | ScFc x | | LLTFGCGTKVEIKSGGGGSEVQLVESGGGLVQPGGSLKLSCAASGFTFNKYAMNWVRQ |
| | (G4S) 3 x | | APGKGMEWVARIRSKYNNYATYYADAVKDRFTISRDDSKNTLYLQMNNLKTEDTAVYY |
| | CD20 99E5 | | CVRAGNFGSSYISYFAYWGQGTLVTVSSGGGGSGGGGSGGGGSQTVVTQEPSLTVSPG |
| | CC x I2L- | | GTVTITCGSSTGAVTSGNYPNWIQKKPGQAPRGLIGGTKFLAPGTPARFSGSLEGGKA |
| | Full | | ALTLSGVQPEDEAEYYCVLYYSNRWVFGSGTKLTVLGGGGSGGGGSGGGGSDKTHTCP |
| | Sequence | | PCPAPELLGGPSVFLFPPKPKDTLMISRTPEVTCVVVDVSHEDPEVKENWYVDGVEVH |
| | | | NAKTKPCEEQYGSTYRCVSVLTVLHQDWLNGKEYKCKVSNKALPAPIEKTISKAKGQP |
| | | | REPQVYTLPPSREEMTKNQVSLTCLVKGFYPSDIAVEWESNGQPENNYKTTPPVLDSD |
| | | | GSFFLYSKLTVDKSRWQQGNVFSCSVMHEALHNHYTQKSLSLSPGKGGGGSGGGGSGG |
| | | | GGSGGGGSGGGGSGGGGSDKTHTCPPCPAPELLGGPSVFLFPPKPKDTLMISRTPEVT |
| | | | CVVVDVSHEDPEVKFNWYVDGVEVHNAKTKPCEEQYGSTYRCVSVLTVLHQDWLNGKE |
| | | | YKCKVSNKALPAPIEKTISKAKGQPREPQVYTLPPSREEMTKNQVSLTCLVKGFYPSD |
| | | | IAVEWESNGQPENNYKTTPPVLDSDGSFFLYSKLTVDKSRWQQGNVFSCSVMHEALHN |
| | | | HYTQKSLSLSPGKGGGGSGGGGSGGGGSQVQLVQSGAEVKKPGASVKVSCKASGYTFT |
| | | | SYWMHWVRQAPGQCLEWIGYITPSTGYTEYNQKFKGRVTMTRDKSTSTVYMELSSLTS |
| | | | EDTAVYYCARVHDYDRAMEYWGQGTTVTVSSGGGGSGGGGSGGGGSDIQMTQSPSSLS |
| | | | ASVGDRVTITCKASQDINKYIAWYQQKPGKGPKLLIYYTSTLQPGVPSRFSGSGSGTD |
| | | | FTFTISSLQPEDIATYYCLQYASYPFTFGCGTRLEIKSGGGGSEVQLVESGGGLVQPG |
| | | | GSLKLSCAASGFTFNKYAMNWVRQAPGKGMEWVARIRSKYNNYATYYADAVKDRFTIS |
| | | | RDDSKNTLYLQMNNLKTEDTAVYYCVRAGNFGSSYISYFAYWGQGTLVTVSSGGGGSG |
| | | | GGGSGGGGSQTVVTQEPSLTVSPGGTVTITCGSSTGAVTSGNYPNWIQKKPGQAPRGL |
| | | | IGGTKFLAPGTPARFSGSLEGGKAALTLSGVQPEDEAEYYCVLYYSNRWVFGSGTKLT |
| | | | VL |
| 2515 | CD22 28- | artificial | QVQLVQSGAEVKKPGASVKVSCKASGYTFTSYGISWVRQAPGQCLEWMGWISAYSGNA |
| | B7N65S CC x | | IYAQKLQGRVTMTRDTSTSTAYMELRSLRSDDTAVYYCARDPDYYGSGSYSDYWGQGT |
| | I2M x | | LVTVSSGGGGSGGGGSGGGGSEIVLTQSPATLSVSPGERATLSCRASQSVSSNLAWYQ |
| | (G4S) 3 x | | QKPGQAPRLLIYGASSRATGIPARFSGSGSGTEFTLTISSLQSEDFAVYYCQQYHSWP |
| | ScFc x | | LLTFGCGTKVEIKSGGGGSEVQLVESGGGLVQPGGSLKLSCAASGFTENKYAMNWVRQ |
| | (G4S) 3 x | | APGKGMEWVARIRSKYNNYATYYADAVKDRFTISRDDSKNTLYLQMNNLKTEDTAVYY |
| | CD20 99E5 | | CVRAGNFGTSYISYWAYWGQGTLVTVSSGGGGSGGGGSGGGGSQTVVTQEPSLTVSPG |
| | CC x I2M- | | GTVTITCGSSTGAVTSGNYPNWVQKKPGQAPRGLIGGTKFLAPGTPARFSGSLSGGKA |
| | Full | | ALTLSGVQPEDEAEYYCVLWYSNRWVFGSGTKLTVLGGGGSGGGGSGGGGSDKTHTCP |
| | Sequence | | PCPAPELLGGPSVFLFPPKPKDTLMISRTPEVTCVVVDVSHEDPEVKENWYVDGVEVH |
| | | | NAKTKPCEEQYGSTYRCVSVLTVLHQDWLNGKEYKCKVSNKALPAPIEKTISKAKGQP |
| | | | REPQVYTLPPSREEMTKNQVSLTCLVKGFYPSDIAVEWESNGQPENNYKTTPPVLDSD |
| | | | GSFFLYSKLTVDKSRWQQGNVFSCSVMHEALHNHYTQKSLSLSPGKGGGGSGGGGSGG |
| | | | GGSGGGGSGGGGSGGGGSDKTHTCPPCPAPELLGGPSVFLFPPKPKDTLMISRTPEVT |
| | | | CVVVDVSHEDPEVKFNWYVDGVEVHNAKTKPCEEQYGSTYRCVSVLTVLHQDWLNGKE |
| | | | YKCKVSNKALPAPIEKTISKAKGQPREPQVYTLPPSREEMTKNQVSLTCLVKGFYPSD |
| | | | IAVEWESNGQPENNYKTTPPVLDSDGSFFLYSKLTVDKSRWQQGNVFSCSVMHEALHN |
| | | | HYTQKSLSLSPGKGGGGSGGGGSGGGGSQVQLVQSGAEVKKPGASVKVSCKASGYTFT |
| | | | SYWMHWVRQAPGQCLEWIGYITPSTGYTEYNQKFKGRVTMTRDKSTSTVYMELSSLTS |
| | | | EDTAVYYCARVHDYDRAMEYWGQGTTVTVSSGGGGSGGGGSGGGGSDIQMTQSPSSLS |
| | | | ASVGDRVTITCKASQDINKYIAWYQQKPGKGPKLLIYYTSTLQPGVPSRFSGSGSGTD |
| | | | FTFTISSLQPEDIATYYCLQYASYPFTFGCGTRLEIKSGGGGSEVQLVESGGGLVQPG |
| | | | GSLKLSCAASGFTFNKYAMNWVRQAPGKGMEWVARIRSKYNNYATYYADAVKDRFTIS |
| | | | RDDSKNTLYLQMNNLKTEDTAVYYCVRAGNFGTSYISYWAYWGQGTLVTVSSGGGGSG |
| | | | GGGSGGGGSQTVVTQEPSLTVSPGGTVTITCGSSTGAVTSGNYPNWVQKKPGQAPRGL |
| | | | IGGTKFLAPGTPARFSGSLSGGKAALTLSGVQPEDEAEYYCVLWYSNRWVFGSGTKLT |
| | | | VL |
| 2516 | CD22 28- | artificial | QVQLVQSGAEVKKPGASVKVSCKASGYTFTSYGISWVRQAPGQCLEWMGWISAYSGNA |
| | B7N65S CC x | | IYAQKLQGRVTMTRDTSTSTAYMELRSLRSDDTAVYYCARDPDYYGSGSYSDYWGQGT |
| | I2M2 x | | LVTVSSGGGGSGGGGSGGGGSEIVLTQSPATLSVSPGERATLSCRASQSVSSNLAWYQ |
| | (G4S) 3 x | | QKPGQAPRLLIYGASSRATGIPARFSGSGSGTEFTLTISSLQSEDFAVYYCQQYHSWP |
| | ScFc x | | LLTFGCGTKVEIKSGGGGSEVQLVESGGGLVQPGGSLKLSCAASGFTENKYAINWVRE |
| | (G4S) 3 x | | APGKGLEWVARIRSKYNNYATYYADAVKDRFTISRDDSKNTAYLQMNNLKTEDTAVYY |
| | CD20 99E5 | | CVRNANFGTSYISYFAYWGQGTLVTVSSGGGGSGGGGSGGGGSQTVVTQEPSLTVSPG |
| | CC x I2M2- | | GTVTLTCGSSTGAVTSGNYPNWVQKKPGQAPRGLIGGTKFLAPGTPARFSGSLLGGKA |
| | Full | | ALTLSGVQPEDEAEYYCVLWYSNRWVFGSGTKLTVLGGGGSGGGGSGGGGSDKTHTCP |
| | Sequence | | PCPAPELLGGPSVFLFPPKPKDTLMISRTPEVTCVVVDVSHEDPEVKFNWYVDGVEVH |
| | | | NAKTKPCEEQYGSTYRCVSVLTVLHQDWLNGKEYKCKVSNKALPAPIEKTISKAKGQP |
| | | | REPQVYTLPPSREEMTKNQVSLTCLVKGFYPSDIAVEWESNGQPENNYKTTPPVLDSD |
| | | | GSFFLYSKLTVDKSRWQQGNVFSCSVMHEALHNHYTQKSLSLSPGKGGGGSGGGGSGG |
| | | | GGSGGGGSGGGGSGGGGSDKTHTCPPCPAPELLGGPSVFLFPPKPKDTLMISRTPEVT |
| | | | CVVVDVSHEDPEVKFNWYVDGVEVHNAKTKPCEEQYGSTYRCVSVLTVLHQDWLNGKE |
| | | | YKCKVSNKALPAPIEKTISKAKGQPREPQVYTLPPSREEMTKNQVSLTCLVKGFYPSD |
| | | | IAVEWESNGQPENNYKTTPPVLDSDGSFFLYSKLTVDKSRWQQGNVFSCSVMHEALHN |
| | | | HYTQKSLSLSPGKGGGGSGGGGSGGGGSQVQLVQSGAEVKKPGASVKVSCKASGYTFT |
| | | | SYWMHWVRQAPGQCLEWIGYITPSTGYTEYNQKFKGRVTMTRDKSTSTVYMELSSLTS |
| | | | EDTAVYYCARVHDYDRAMEYWGQGTTVTVSSGGGGSGGGGSGGGGSDIQMTQSPSSLS |
| | | | ASVGDRVTITCKASQDINKYIAWYQQKPGKGPKLLIYYTSTLQPGVPSRFSGSGSGTD |
| | | | FTFTISSLQPEDIATYYCLQYASYPFTFGCGTRLEIKSGGGGSEVQLVESGGGLVQPG |
| | | | GSLKLSCAASGFTFNKYAINWVREAPGKGLEWVARIRSKYNNYATYYADAVKDRFTIS |
| | | | RDDSKNTAYLQMNNLKTEDTAVYYCVRNANFGTSYISYFAYWGQGTLVTVSSGGGGSG |
| | | | GGGSGGGGSQTVVTQEPSLTVSPGGTVTLTCGSSTGAVTSGNYPNWVQKKPGQAPRGL |
| | | | IGGTKFLAPGTPARFSGSLLGGKAALTLSGVQPEDEAEYYCVLWYSNRWVFGSGTKLT |
| | | | VL |
| 2517 | CS | artificial | DIQMTQSPSSLSASVGDRVTITCKASQDVSTAVAWYQQKPGKAPKLLIYSASYRYTGV |
| | PDL241.12.LH | | PDRFTGSGSGTDFTLTISSLQPEDFATYYCQQHYSTPPYTFGCGTKVEIKRGGGGSGG |
| | CCx BC A7 | | GGSGGGGSQVQLVQSGAEVKKPGASVKVSCKASGYAFSSSWMNWVRQAPGQCLEWIGR |
| | 27-C4-G7 CC | | IYPGDADAKYNAKFKGKATLTADKSTSTAYMELSSLRSEDTAVYYCARSTMIATGAMD |
| | x I2Cx scFc | | YWGQGTLVTVSSSGGGGSQVQLVQSGAEVKKPGASVKVSCKASGYTFTNHIIHWVRQA |
| | -Full | | PGQCLEWMGYINPYPGYHAYNEKFQGRATMTSDTSTSTVYMELSSLRSEDTAVYYCAR |
| | Sequence | | DGYYRDTDVLDYWGQGTLVTVSSGGGGSGGGGSGGGGSDIQMTQSPSSLSASVGDRVT |
| | | | ITCQASQDISNYLNWYQQKPGKAPKLLIYYTSRLHTGVPSRFSGSGSGTDFTFTISSL |
| | | | EPEDIATYYCQQGNTLPWTFGCGTKVEIKSGGGGSEVQLVESGGGLVQPGGSLKLSCA |
| | | | ASGFTFNKYAMNWVRQAPGKGLEWVARIRSKYNNYATYYADSVKDRFTISRDDSKNTA |
| | | | YLQMNNLKTEDTAVYYCVRHGNFGNSYISYWAYWGQGTLVTVSSGGGGSGGGGSGGGG |
| | | | SQTVVTQEPSLTVSPGGTVTLTCGSSTGAVTSGNYPNWVQQKPGQAPRGLIGGTKFLA |
| | | | PGTPARFSGSLLGGKAALTLSGVQPEDEAEYYCVLWYSNRWVFGGGTKLTVLGGGGDK |
| | | | THTCPPCPAPELLGGPSVFLFPPKPKDTLMISRTPEVTCVVVDVSHEDPEVKENWYVD |
| | | | GVEVHNAKTKPCEEQYGSTYRCVSVLTVLHQDWLNGKEYKCKVSNKALPAPIEKTISK |
| | | | AKGQPREPQVYTLPPSREEMTKNQVSLTCLVKGFYPSDIAVEWESNGQPENNYKTTPP |
| | | | VLDSDGSFFLYSKLTVDKSRWQQGNVFSCSVMHEALHNHYTQKSLSLSPGKGGGGSGG |
| | | | GGSGGGGSGGGGSGGGGSGGGGSDKTHTCPPCPAPELLGGPSVFLFPPKPKDTLMISR |
| | | | TPEVTCVVVDVSHEDPEVKFNWYVDGVEVHNAKTKPCEEQYGSTYRCVSVLTVLHQDW |
| | | | LNGKEYKCKVSNKALPAPIEKTISKAKGQPREPQVYTLPPSREEMTKNQVSLTCLVKG |
| | | | FYPSDIAVEWESNGQPENNYKTTPPVLDSDGSFFLYSKLTVDKSRWQQGNVFSCSVMH |
| | | | EALHNHYTQKSLSLSPGK |
| 2518 | CS | artificial | DIQMTQSPSSLSASVGDRVTITCKASQDVSTAVAWYQQKPGKAPKLLIYSASYRYTGV |
| | PDL241.12.LH | | PDRFTGSGSGTDFTLTISSLQPEDFATYYCQQHYSTPPYTFGCGTKVEIKRGGGGSGG |
| | CCx BC A7 | | GGSGGGGSQVQLVQSGAEVKKPGASVKVSCKASGYAFSSSWMNWVRQAPGQCLEWIGR |
| | 27-C4-G7 CC | | IYPGDADAKYNAKFKGKATLTADKSTSTAYMELSSLRSEDTAVYYCARSTMIATGAMD |
| | x I2Ex scFc | | YWGQGTLVTVSSSGGGGSQVQLVQSGAEVKKPGASVKVSCKASGYTFTNHIIHWVRQA |
| | -Full | | PGQCLEWMGYINPYPGYHAYNEKFQGRATMTSDTSTSTVYMELSSLRSEDTAVYYCAR |
| | Sequence | | DGYYRDTDVLDYWGQGTLVTVSSGGGGSGGGGSGGGGSDIQMTQSPSSLSASVGDRVT |
| | | | ITCQASQDISNYLNWYQQKPGKAPKLLIYYTSRLHTGVPSRFSGSGSGTDFTFTISSL |
| | | | EPEDIATYYCQQGNTLPWTFGCGTKVEIKSGGGGSEVQLVESGGGLVQPGGSLKLSCA |
| | | | ASGFTFNKYAINWVRQAPGKGLEWVARIRSKYNNYATYYADAVKDRFTISRDDSKNTV |
| | | | YLQMNNLKTEDTAVYYCARAGNFGSSYISYWAYWGQGTLVTVSSGGGGSGGGGSGGGG |
| | | | SQTVVTQEPSLTVSPGGTVTITCGSSTGAVTSGNYPNWVQKKPGQAPRGLIGGTKFLA |
| | | | PGTPARFSGSLSGGKAALTLSGVQPEDEAEYYCVLWYSNRWVFGSGTKLTVLGGGGDK |
| | | | THTCPPCPAPELLGGPSVFLFPPKPKDTLMISRTPEVTCVVVDVSHEDPEVKENWYVD |
| | | | GVEVHNAKTKPCEEQYGSTYRCVSVLTVLHQDWLNGKEYKCKVSNKALPAPIEKTISK |
| | | | AKGQPREPQVYTLPPSREEMTKNQVSLTCLVKGFYPSDIAVEWESNGQPENNYKTTPP |
| | | | VLDSDGSFFLYSKLTVDKSRWQQGNVFSCSVMHEALHNHYTQKSLSLSPGKGGGGSGG |
| | | | GGSGGGGSGGGGSGGGGSGGGGSDKTHTCPPCPAPELLGGPSVFLFPPKPKDTLMISR |
| | | | TPEVTCVVVDVSHEDPEVKFNWYVDGVEVHNAKTKPCEEQYGSTYRCVSVLTVLHQDW |
| | | | LNGKEYKCKVSNKALPAPIEKTISKAKGQPREPQVYTLPPSREEMTKNQVSLTCLVKG |
| | | | FYPSDIAVEWESNGQPENNYKTTPPVLDSDGSFFLYSKLTVDKSRWQQGNVFSCSVMH |
| | | | EALHNHYTQKSLSLSPGK |
| 2519 | CS | artificial | DIQMTQSPSSLSASVGDRVTITCKASQDVSTAVAWYQQKPGKAPKLLIYSASYRYTGV |
| | PDL241.12.LH | | PDRFTGSGSGTDFTLTISSLQPEDFATYYCQQHYSTPPYTFGCGTKVEIKRGGGGSGG |
| | CCx BC A7 | | GGSGGGGSQVQLVQSGAEVKKPGASVKVSCKASGYAFSSSWMNWVRQAPGQCLEWIGR |
| | 27-C4-G7 CC | | IYPGDADAKYNAKFKGKATLTADKSTSTAYMELSSLRSEDTAVYYCARSTMIATGAMD |
| | x I2Kx scFc | | YWGQGTLVTVSSSGGGGSQVQLVQSGAEVKKPGASVKVSCKASGYTFTNHIIHWVRQA |
| | -Full | | PGQCLEWMGYINPYPGYHAYNEKFQGRATMTSDTSTSTVYMELSSLRSEDTAVYYCAR |
| | Sequence | | DGYYRDTDVLDYWGQGTLVTVSSGGGGSGGGGSGGGGSDIQMTQSPSSLSASVGDRVT |
| | | | ITCQASQDISNYLNWYQQKPGKAPKLLIYYTSRLHTGVPSRFSGSGSGTDFTFTISSL |
| | | | EPEDIATYYCQQGNTLPWTFGCGTKVEIKSGGGGSEVQLVESGGGLVQPGGSLKLSCA |
| | | | ASGFTFNKYAMNWVRQAPGKGLEWVARIRSKYNNYATYYAEAVKGRFTISRDDSKNTV |
| | | | YLQMNNLKTEDTAVYYCVRNENIGTSYISYWAYWGQGTLVTVSSGGGGSGGGGSGGGG |
| | | | SQTVVTQEPSLTVSPGGTVTITCGSSTGAVTSGNYPNWVQQKPGQAPRGLIGGTKFLA |
| | | | PGTPARFSGSLSGGKAALTLSGVQPEDEAEYYCVLWYSNRWVFGSGTKLTVLGGGGDK |
| | | | THTCPPCPAPELLGGPSVFLFPPKPKDTLMISRTPEVTCVVVDVSHEDPEVKENWYVD |
| | | | GVEVHNAKTKPCEEQYGSTYRCVSVLTVLHQDWLNGKEYKCKVSNKALPAPIEKTISK |
| | | | AKGQPREPQVYTLPPSREEMTKNQVSLTCLVKGFYPSDIAVEWESNGQPENNYKTTPP |
| | | | VLDSDGSFFLYSKLTVDKSRWQQGNVFSCSVMHEALHNHYTQKSLSLSPGKGGGGSGG |
| | | | GGSGGGGSGGGGSGGGGSGGGGSDKTHTCPPCPAPELLGGPSVFLFPPKPKDTLMISR |
| | | | TPEVTCVVVDVSHEDPEVKFNWYVDGVEVHNAKTKPCEEQYGSTYRCVSVLTVLHQDW |
| | | | LNGKEYKCKVSNKALPAPIEKTISKAKGQPREPQVYTLPPSREEMTKNQVSLTCLVKG |
| | | | FYPSDIAVEWESNGQPENNYKTTPPVLDSDGSFFLYSKLTVDKSRWQQGNVFSCSVMH |
| | | | EALHNHYTQKSLSLSPGK |
| 2520 | CS | artificial | DIQMTQSPSSLSASVGDRVTITCKASQDVSTAVAWYQQKPGKAPKLLIYSASYRYTGV |
| | PDL241.12.LH | | PDRFTGSGSGTDFTLTISSLQPEDFATYYCQQHYSTPPYTFGCGTKVEIKRGGGGSGG |
| | CCx BC A7 | | GGSGGGGSQVQLVQSGAEVKKPGASVKVSCKASGYAFSSSWMNWVRQAPGQCLEWIGR |
| | 27-C4-G7 CC | | IYPGDADAKYNAKFKGKATLTADKSTSTAYMELSSLRSEDTAVYYCARSTMIATGAMD |
| | x I2Lx scFc | | YWGQGTLVTVSSSGGGGSQVQLVQSGAEVKKPGASVKVSCKASGYTFTNHIIHWVRQA |
| | -Full | | PGQCLEWMGYINPYPGYHAYNEKFQGRATMTSDTSTSTVYMELSSLRSEDTAVYYCAR |
| | Sequence | | DGYYRDTDVLDYWGQGTLVTVSSGGGGSGGGGSGGGGSDIQMTQSPSSLSASVGDRVT |
| | | | ITCQASQDISNYLNWYQQKPGKAPKLLIYYTSRLHTGVPSRFSGSGSGTDFTFTISSL |
| | | | EPEDIATYYCQQGNTLPWTFGCGTKVEIKSGGGGSEVQLVESGGGLVQPGGSLKLSCA |
| | | | ASGFTFNKYAMNWVRQAPGKGMEWVARIRSKYNNYATYYADAVKDRFTISRDDSKNTL |
| | | | YLQMNNLKTEDTAVYYCVRAGNFGSSYISYFAYWGQGTLVTVSSGGGGSGGGGSGGGG |
| | | | SQTVVTQEPSLTVSPGGTVTITCGSSTGAVTSGNYPNWIQKKPGQAPRGLIGGTKFLA |
| | | | PGTPARFSGSLEGGKAALTLSGVQPEDEAEYYCVLYYSNRWVFGSGTKLTVLGGGGDK |
| | | | THTCPPCPAPELLGGPSVFLFPPKPKDTLMISRTPEVTCVVVDVSHEDPEVKENWYVD |
| | | | GVEVHNAKTKPCEEQYGSTYRCVSVLTVLHQDWLNGKEYKCKVSNKALPAPIEKTISK |
| | | | AKGQPREPQVYTLPPSREEMTKNQVSLTCLVKGFYPSDIAVEWESNGQPENNYKTTPP |
| | | | VLDSDGSFFLYSKLTVDKSRWQQGNVFSCSVMHEALHNHYTQKSLSLSPGKGGGGSGG |
| | | | GGSGGGGSGGGGSGGGGSGGGGSDKTHTCPPCPAPELLGGPSVFLFPPKPKDTLMISR |
| | | | TPEVTCVVVDVSHEDPEVKFNWYVDGVEVHNAKTKPCEEQYGSTYRCVSVLTVLHQDW |
| | | | LNGKEYKCKVSNKALPAPIEKTISKAKGQPREPQVYTLPPSREEMTKNQVSLTCLVKG |
| | | | FYPSDIAVEWESNGQPENNYKTTPPVLDSDGSFFLYSKLTVDKSRWQQGNVFSCSVMH |
| | | | EALHNHYTQKSLSLSPGK |
| 2521 | CS | artificial | DIQMTQSPSSLSASVGDRVTITCKASQDVSTAVAWYQQKPGKAPKLLIYSASYRYTGV |
| | PDL241.12.LH | | PDRFTGSGSGTDFTLTISSLQPEDFATYYCQQHYSTPPYTFGCGTKVEIKRGGGGSGG |
| | CCx BC A7 | | GGSGGGGSQVQLVQSGAEVKKPGASVKVSCKASGYAFSSSWMNWVRQAPGQCLEWIGR |
| | 27-C4-G7 CC | | IYPGDADAKYNAKFKGKATLTADKSTSTAYMELSSLRSEDTAVYYCARSTMIATGAMD |
| | x I2M2x | | YWGQGTLVTVSSSGGGGSQVQLVQSGAEVKKPGASVKVSCKASGYTFTNHIIHWVRQA |
| | scFc-Full | | PGQCLEWMGYINPYPGYHAYNEKFQGRATMTSDTSTSTVYMELSSLRSEDTAVYYCAR |
| | Sequence | | DGYYRDTDVLDYWGQGTLVTVSSGGGGSGGGGSGGGGSDIQMTQSPSSLSASVGDRVT |
| | | | ITCQASQDISNYLNWYQQKPGKAPKLLIYYTSRLHTGVPSRFSGSGSGTDFTFTISSL |
| | | | EPEDIATYYCQQGNTLPWTFGCGTKVEIKSGGGGSEVQLVESGGGLVQPGGSLKLSCA |
| | | | ASGFTFNKYAINWVREAPGKGLEWVARIRSKYNNYATYYADAVKDRFTISRDDSKNTA |
| | | | YLQMNNLKTEDTAVYYCVRNANFGTSYISYFAYWGQGTLVTVSSGGGGSGGGGSGGGG |
| | | | SQTVVTQEPSLTVSPGGTVTLTCGSSTGAVTSGNYPNWVQKKPGQAPRGLIGGTKFLA |
| | | | PGTPARFSGSLLGGKAALTLSGVQPEDEAEYYCVLWYSNRWVFGSGTKLTVLGGGGDK |
| | | | THTCPPCPAPELLGGPSVFLFPPKPKDTLMISRTPEVTCVVVDVSHEDPEVKFNWYVD |
| | | | GVEVHNAKTKPCEEQYGSTYRCVSVLTVLHQDWLNGKEYKCKVSNKALPAPIEKTISK |
| | | | AKGQPREPQVYTLPPSREEMTKNQVSLTCLVKGFYPSDIAVEWESNGQPENNYKTTPP |
| | | | VLDSDGSFFLYSKLTVDKSRWQQGNVFSCSVMHEALHNHYTQKSLSLSPGKGGGGSGG |
| | | | GGSGGGGSGGGGSGGGGSGGGGSDKTHTCPPCPAPELLGGPSVFLFPPKPKDTLMISR |
| | | | TPEVTCVVVDVSHEDPEVKFNWYVDGVEVHNAKTKPCEEQYGSTYRCVSVLTVLHQDW |
| | | | LNGKEYKCKVSNKALPAPIEKTISKAKGQPREPQVYTLPPSREEMTKNQVSLTCLVKG |
| | | | FYPSDIAVEWESNGQPENNYKTTPPVLDSDGSFFLYSKLTVDKSRWQQGNVFSCSVMH |
| | | | EALHNHYTQKSLSLSPGK |
| 2522 | CS | artificial | DIQMTQSPSSLSASVGDRVTITCKASQDVSTAVAWYQQKPGKAPKLLIYSASYRYTGV |
| | PDL241.12.LH | | PDRFTGSGSGTDFTLTISSLQPEDFATYYCQQHYSTPPYTFGCGTKVEIKRGGGGSGG |
| | CCx BC A7 | | GGSGGGGSQVQLVQSGAEVKKPGASVKVSCKASGYAFSSSWMNWVRQAPGQCLEWIGR |
| | 27-C4-G7 CC | | IYPGDADAKYNAKFKGKATLTADKSTSTAYMELSSLRSEDTAVYYCARSTMIATGAMD |
| | x I2Mx scFc | | YWGQGTLVTVSSSGGGGSQVQLVQSGAEVKKPGASVKVSCKASGYTFTNHIIHWVRQA |
| | -Full | | PGQCLEWMGYINPYPGYHAYNEKFQGRATMTSDTSTSTVYMELSSLRSEDTAVYYCAR |
| | Sequence | | DGYYRDTDVLDYWGQGTLVTVSSGGGGSGGGGSGGGGSDIQMTQSPSSLSASVGDRVT |
| | | | ITCQASQDISNYLNWYQQKPGKAPKLLIYYTSRLHTGVPSRFSGSGSGTDFTFTISSL |
| | | | EPEDIATYYCQQGNTLPWTFGCGTKVEIKSGGGGSEVQLVESGGGLVQPGGSLKLSCA |
| | | | ASGFTFNKYAMNWVRQAPGKGMEWVARIRSKYNNYATYYADAVKDRFTISRDDSKNTL |
| | | | YLQMNNLKTEDTAVYYCVRAGNFGTSYISYWAYWGQGTLVTVSSGGGGSGGGGSGGGG |
| | | | SQTVVTQEPSLTVSPGGTVTITCGSSTGAVTSGNYPNWVQKKPGQAPRGLIGGTKFLA |
| | | | PGTPARFSGSLSGGKAALTLSGVQPEDEAEYYCVLWYSNRWVFGSGTKLTVLGGGGDK |
| | | | THTCPPCPAPELLGGPSVFLFPPKPKDTLMISRTPEVTCVVVDVSHEDPEVKENWYVD |
| | | | GVEVHNAKTKPCEEQYGSTYRCVSVLTVLHQDWLNGKEYKCKVSNKALPAPIEKTISK |
| | | | AKGQPREPQVYTLPPSREEMTKNQVSLTCLVKGFYPSDIAVEWESNGQPENNYKTTPP |
| | | | VLDSDGSFFLYSKLTVDKSRWQQGNVFSCSVMHEALHNHYTQKSLSLSPGKGGGGSGG |
| | | | GGSGGGGSGGGGSGGGGSGGGGSDKTHTCPPCPAPELLGGPSVFLFPPKPKDTLMISR |
| | | | TPEVTCVVVDVSHEDPEVKFNWYVDGVEVHNAKTKPCEEQYGSTYRCVSVLTVLHQDW |
| | | | LNGKEYKCKVSNKALPAPIEKTISKAKGQPREPQVYTLPPSREEMTKNQVSLTCLVKG |
| | | | FYPSDIAVEWESNGQPENNYKTTPPVLDSDGSFFLYSKLTVDKSRWQQGNVFSCSVMH |
| | | | EALHNHYTQKSLSLSPGK |
| 2523 | FL 7-A8 CC | artificial | QVTLKESGPTLVKPTETLTLTCTLSGFSLNNARMGVSWIRQPPGKCLEWLAHIFSNDE |
| | xCD123 24- | | KSYSTSLKNRLTISKDSSKTQVVLTMTNVDPVDTATYYCARIVGYGSGWYGFFDYWGQ |
| | B4-f NK CC | | GTLVTVSSGGGGSGGGGSGGGGSDIQMTQSPSSLSASVGDRVTITCRASQGIRNDLGW |
| | x I2C x | | YQQKPGKAPKRLIYAASTLQSGVPSRFSGSGSGTEFTLTISSLQPEDFATYYCLQHNS |
| | scFc-Full | | YPLTFGCGTKVEIKSGGGGSEVQLLESGGGLVQPGGSLRLSCAASGFTFSHYAMSWVR |
| | Sequence | | QAPGKCLEWVSAVSGGGDKTLYADAVKGRFTISRDNSKNTLFLQMNSLRAEDTAIYYC |
| | | | ARLRGFYYGMDVWGQGTTVTVSSGGGGSGGGGSGGGGSDIVLTQSPLSLPVTPGEPAS |
| | | | ISCRSSQSLLHSNKYNYLDWYLQKPGQSPQLLIYLGSNRASGVPDRFSGSGSGTDFTL |
| | | | KISRVEAEDVGVYYCMQALQTPPITFGCGTRLEIKSGGGGSEVQLVESGGGLVQPGGS |
| | | | LKLSCAASGFTFNKYAMNWVRQAPGKGLEWVARIRSKYNNYATYYADSVKDRFTISRD |
| | | | DSKNTAYLQMNNLKTEDTAVYYCVRHGNFGNSYISYWAYWGQGTLVTVSSGGGGSGGG |
| | | | GSGGGGSQTVVTQEPSLTVSPGGTVTLTCGSSTGAVTSGNYPNWVQQKPGQAPRGLIG |
| | | | GTKFLAPGTPARFSGSLLGGKAALTLSGVQPEDEAEYYCVLWYSNRWVFGGGTKLTVL |
| | | | GGGGDKTHTCPPCPAPELLGGPSVFLFPPKPKDTLMISRTPEVTCVVVDVSHEDPEVK |
| | | | FNWYVDGVEVHNAKTKPCEEQYGSTYRCVSVLTVLHQDWLNGKEYKCKVSNKALPAPI |
| | | | EKTISKAKGQPREPQVYTLPPSREEMTKNQVSLTCLVKGFYPSDIAVEWESNGQPENN |
| | | | YKTTPPVLDSDGSFFLYSKLTVDKSRWQQGNVFSCSVMHEALHNHYTQKSLSLSPGKG |
| | | | GGGSGGGGSGGGGSGGGGSGGGGSGGGGSDKTHTCPPCPAPELLGGPSVFLFPPKPKD |
| | | | TLMISRTPEVTCVVVDVSHEDPEVKENWYVDGVEVHNAKTKPCEEQYGSTYRCVSVLT |
| | | | VLHQDWLNGKEYKCKVSNKALPAPIEKTISKAKGQPREPQVYTLPPSREEMTKNQVSL |
| | | | TCLVKGFYPSDIAVEWESNGQPENNYKTTPPVLDSDGSFFLYSKLTVDKSRWQQGNVF |
| | | | SCSVMHEALHNHYTQKSLSLSPGK |
| 2524 | FL 7-A8 CC | artificial | QVTLKESGPTLVKPTETLTLTCTLSGFSLNNARMGVSWIRQPPGKCLEWLAHIFSNDE |
| | xCD123 24- | | KSYSTSLKNRLTISKDSSKTQVVLTMTNVDPVDTATYYCARIVGYGSGWYGFFDYWGQ |
| | B4-f NK CC | | GTLVTVSSGGGGSGGGGSGGGGSDIQMTQSPSSLSASVGDRVTITCRASQGIRNDLGW |
| | x I2E X | | YQQKPGKAPKRLIYAASTLQSGVPSRFSGSGSGTEFTLTISSLQPEDFATYYCLQHNS |
| | scFc-Full | | YPLTFGCGTKVEIKSGGGGSEVQLLESGGGLVQPGGSLRLSCAASGFTFSHYAMSWVR |
| | Sequence | | QAPGKCLEWVSAVSGGGDKTLYADAVKGRFTISRDNSKNTLFLQMNSLRAEDTAIYYC |
| | | | ARLRGFYYGMDVWGQGTTVTVSSGGGGSGGGGSGGGGSDIVLTQSPLSLPVTPGEPAS |
| | | | ISCRSSQSLLHSNKYNYLDWYLQKPGQSPQLLIYLGSNRASGVPDRFSGSGSGTDFTL |
| | | | KISRVEAEDVGVYYCMQALQTPPITFGCGTRLEIKSGGGGSEVQLVESGGGLVQPGGS |
| | | | LKLSCAASGFTFNKYAINWVRQAPGKGLEWVARIRSKYNNYATYYADAVKDRFTISRD |
| | | | DSKNTVYLQMNNLKTEDTAVYYCARAGNFGSSYISYWAYWGQGTLVTVSSGGGGSGGG |
| | | | GSGGGGSQTVVTQEPSLTVSPGGTVTITCGSSTGAVTSGNYPNWVQKKPGQAPRGLIG |
| | | | GTKFLAPGTPARFSGSLSGGKAALTLSGVQPEDEAEYYCVLWYSNRWVFGSGTKLTVL |
| | | | GGGGDKTHTCPPCPAPELLGGPSVFLFPPKPKDTLMISRTPEVTCVVVDVSHEDPEVK |
| | | | FNWYVDGVEVHNAKTKPCEEQYGSTYRCVSVLTVLHQDWLNGKEYKCKVSNKALPAPI |
| | | | EKTISKAKGQPREPQVYTLPPSREEMTKNQVSLTCLVKGFYPSDIAVEWESNGQPENN |
| | | | YKTTPPVLDSDGSFFLYSKLTVDKSRWQQGNVFSCSVMHEALHNHYTQKSLSLSPGKG |
| | | | GGGSGGGGSGGGGSGGGGSGGGGSGGGGSDKTHTCPPCPAPELLGGPSVFLFPPKPKD |
| | | | TLMISRTPEVTCVVVDVSHEDPEVKENWYVDGVEVHNAKTKPCEEQYGSTYRCVSVLT |
| | | | VLHQDWLNGKEYKCKVSNKALPAPIEKTISKAKGQPREPQVYTLPPSREEMTKNQVSL |
| | | | TCLVKGFYPSDIAVEWESNGQPENNYKTTPPVLDSDGSFFLYSKLTVDKSRWQQGNVF |
| | | | SCSVMHEALHNHYTQKSLSLSPGK |
| 2525 | FL 7-A8 CC | artificial | QVTLKESGPTLVKPTETLTLTCTLSGFSLNNARMGVSWIRQPPGKCLEWLAHIFSNDE |
| | xCD123 24- | | KSYSTSLKNRLTISKDSSKTQVVLTMTNVDPVDTATYYCARIVGYGSGWYGFFDYWGQ |
| | B4-f NK CC | | GTLVTVSSGGGGSGGGGSGGGGSDIQMTQSPSSLSASVGDRVTITCRASQGIRNDLGW |
| | x I2K X | | YQQKPGKAPKRLIYAASTLQSGVPSRFSGSGSGTEFTLTISSLQPEDFATYYCLQHNS |
| | scFc-Full | | YPLTFGCGTKVEIKSGGGGSEVQLLESGGGLVQPGGSLRLSCAASGFTFSHYAMSWVR |
| | Sequence | | QAPGKCLEWVSAVSGGGDKTLYADAVKGRFTISRDNSKNTLFLQMNSLRAEDTAIYYC |
| | | | ARLRGFYYGMDVWGQGTTVTVSSGGGGSGGGGSGGGGSDIVLTQSPLSLPVTPGEPAS |
| | | | ISCRSSQSLLHSNKYNYLDWYLQKPGQSPQLLIYLGSNRASGVPDRFSGSGSGTDFTL |
| | | | KISRVEAEDVGVYYCMQALQTPPITFGCGTRLEIKSGGGGSEVQLVESGGGLVQPGGS |
| | | | LKLSCAASGFTFNKYAMNWVRQAPGKGLEWVARIRSKYNNYATYYAEAVKGRFTISRD |
| | | | DSKNTVYLQMNNLKTEDTAVYYCVRNENIGTSYISYWAYWGQGTLVTVSSGGGGSGGG |
| | | | GSGGGGSQTVVTQEPSLTVSPGGTVTITCGSSTGAVTSGNYPNWVQQKPGQAPRGLIG |
| | | | GTKFLAPGTPARFSGSLSGGKAALTLSGVQPEDEAEYYCVLWYSNRWVFGSGTKLTVL |
| | | | GGGGDKTHTCPPCPAPELLGGPSVFLFPPKPKDTLMISRTPEVTCVVVDVSHEDPEVK |
| | | | FNWYVDGVEVHNAKTKPCEEQYGSTYRCVSVLTVLHQDWLNGKEYKCKVSNKALPAPI |
| | | | EKTISKAKGQPREPQVYTLPPSREEMTKNQVSLTCLVKGFYPSDIAVEWESNGQPENN |
| | | | YKTTPPVLDSDGSFFLYSKLTVDKSRWQQGNVFSCSVMHEALHNHYTQKSLSLSPGKG |
| | | | GGGSGGGGSGGGGSGGGGSGGGGSGGGGSDKTHTCPPCPAPELLGGPSVFLFPPKPKD |
| | | | TLMISRTPEVTCVVVDVSHEDPEVKENWYVDGVEVHNAKTKPCEEQYGSTYRCVSVLT |
| | | | VLHQDWLNGKEYKCKVSNKALPAPIEKTISKAKGQPREPQVYTLPPSREEMTKNQVSL |
| | | | TCLVKGFYPSDIAVEWESNGQPENNYKTTPPVLDSDGSFFLYSKLTVDKSRWQQGNVF |
| | | | SCSVMHEALHNHYTQKSLSLSPGK |
| 2526 | FL 7-A8 CC | artificial | QVTLKESGPTLVKPTETLTLTCTLSGFSLNNARMGVSWIRQPPGKCLEWLAHIFSNDE |
| | xCD123 24- | | KSYSTSLKNRLTISKDSSKTQVVLTMTNVDPVDTATYYCARIVGYGSGWYGFFDYWGQ |
| | B4-f NK CC | | GTLVTVSSGGGGSGGGGSGGGGSDIQMTQSPSSLSASVGDRVTITCRASQGIRNDLGW |
| | x I2L x | | YQQKPGKAPKRLIYAASTLQSGVPSRFSGSGSGTEFTLTISSLQPEDFATYYCLQHNS |
| | scFc-Full | | YPLTFGCGTKVEIKSGGGGSEVQLLESGGGLVQPGGSLRLSCAASGFTFSHYAMSWVR |
| | Sequence | | QAPGKCLEWVSAVSGGGDKTLYADAVKGRFTISRDNSKNTLFLQMNSLRAEDTAIYYC |
| | | | ARLRGFYYGMDVWGQGTTVTVSSGGGGSGGGGSGGGGSDIVLTQSPLSLPVTPGEPAS |
| | | | ISCRSSQSLLHSNKYNYLDWYLQKPGQSPQLLIYLGSNRASGVPDRFSGSGSGTDFTL |
| | | | KISRVEAEDVGVYYCMQALQTPPITFGCGTRLEIKSGGGGSEVQLVESGGGLVQPGGS |
| | | | LKLSCAASGFTFNKYAMNWVRQAPGKGMEWVARIRSKYNNYATYYADAVKDRFTISRD |
| | | | DSKNTLYLQMNNLKTEDTAVYYCVRAGNFGSSYISYFAYWGQGTLVTVSSGGGGSGGG |
| | | | GSGGGGSQTVVTQEPSLTVSPGGTVTITCGSSTGAVTSGNYPNWIQKKPGQAPRGLIG |
| | | | GTKFLAPGTPARFSGSLEGGKAALTLSGVQPEDEAEYYCVLYYSNRWVFGSGTKLTVL |
| | | | GGGGDKTHTCPPCPAPELLGGPSVFLFPPKPKDTLMISRTPEVTCVVVDVSHEDPEVK |
| | | | FNWYVDGVEVHNAKTKPCEEQYGSTYRCVSVLTVLHQDWLNGKEYKCKVSNKALPAPI |
| | | | EKTISKAKGQPREPQVYTLPPSREEMTKNQVSLTCLVKGFYPSDIAVEWESNGQPENN |
| | | | YKTTPPVLDSDGSFFLYSKLTVDKSRWQQGNVFSCSVMHEALHNHYTQKSLSLSPGKG |
| | | | GGGSGGGGSGGGGSGGGGSGGGGSGGGGSDKTHTCPPCPAPELLGGPSVFLFPPKPKD |
| | | | TLMISRTPEVTCVVVDVSHEDPEVKFNWYVDGVEVHNAKTKPCEEQYGSTYRCVSVLT |
| | | | VLHQDWLNGKEYKCKVSNKALPAPIEKTISKAKGQPREPQVYTLPPSREEMTKNQVSL |
| | | | TCLVKGFYPSDIAVEWESNGQPENNYKTTPPVLDSDGSFFLYSKLTVDKSRWQQGNVE |
| | | | SCSVMHEALHNHYTQKSLSLSPGK |
| 2527 | FL 7-A8 CC | artificial | QVTLKESGPTLVKPTETLTLTCTLSGFSLNNARMGVSWIRQPPGKCLEWLAHIFSNDE |
| | xCD123 24- | | KSYSTSLKNRLTISKDSSKTQVVLTMTNVDPVDTATYYCARIVGYGSGWYGFFDYWGQ |
| | B4-f NK CC | | GTLVTVSSGGGGSGGGGSGGGGSDIQMTQSPSSLSASVGDRVTITCRASQGIRNDLGW |
| | x I2M x | | YQQKPGKAPKRLIYAASTLQSGVPSRFSGSGSGTEFTLTISSLQPEDFATYYCLQHNS |
| | scFc-Full | | YPLTFGCGTKVEIKSGGGGSEVQLLESGGGLVQPGGSLRLSCAASGFTFSHYAMSWVR |
| | Sequence | | QAPGKCLEWVSAVSGGGDKTLYADAVKGRFTISRDNSKNTLFLQMNSLRAEDTAIYYC |
| | | | ARLRGFYYGMDVWGQGTTVTVSSGGGGSGGGGSGGGGSDIVLTQSPLSLPVTPGEPAS |
| | | | ISCRSSQSLLHSNKYNYLDWYLQKPGQSPQLLIYLGSNRASGVPDRFSGSGSGTDFTL |
| | | | KISRVEAEDVGVYYCMQALQTPPITFGCGTRLEIKSGGGGSEVQLVESGGGLVQPGGS |
| | | | LKLSCAASGFTFNKYAMNWVRQAPGKGMEWVARIRSKYNNYATYYADAVKDRFTISRD |
| | | | DSKNTLYLQMNNLKTEDTAVYYCVRAGNFGTSYISYWAYWGQGTLVTVSSGGGGSGGG |
| | | | GSGGGGSQTVVTQEPSLTVSPGGTVTITCGSSTGAVTSGNYPNWVQKKPGQAPRGLIG |
| | | | GTKFLAPGTPARFSGSLSGGKAALTLSGVQPEDEAEYYCVLWYSNRWVFGSGTKLTVL |
| | | | GGGGDKTHTCPPCPAPELLGGPSVFLFPPKPKDTLMISRTPEVTCVVVDVSHEDPEVK |
| | | | FNWYVDGVEVHNAKTKPCEEQYGSTYRCVSVLTVLHQDWLNGKEYKCKVSNKALPAPI |
| | | | EKTISKAKGQPREPQVYTLPPSREEMTKNQVSLTCLVKGFYPSDIAVEWESNGQPENN |
| | | | YKTTPPVLDSDGSFFLYSKLTVDKSRWQQGNVFSCSVMHEALHNHYTQKSLSLSPGKG |
| | | | GGGSGGGGSGGGGSGGGGSGGGGSGGGGSDKTHTCPPCPAPELLGGPSVFLFPPKPKD |
| | | | TLMISRTPEVTCVVVDVSHEDPEVKFNWYVDGVEVHNAKTKPCEEQYGSTYRCVSVLT |
| | | | VLHQDWLNGKEYKCKVSNKALPAPIEKTISKAKGQPREPQVYTLPPSREEMTKNQVSL |
| | | | TCLVKGFYPSDIAVEWESNGQPENNYKTTPPVLDSDGSFFLYSKLTVDKSRWQQGNVF |
| | | | SCSVMHEALHNHYTQKSLSLSPGK |
| 2528 | FL 7-A8 CC | artificial | QVTLKESGPTLVKPTETLTLTCTLSGFSLNNARMGVSWIRQPPGKCLEWLAHIFSNDE |
| | xCD123 24- | | KSYSTSLKNRLTISKDSSKTQVVLTMTNVDPVDTATYYCARIVGYGSGWYGFFDYWGQ |
| | B4-f NK CC | | GTLVTVSSGGGGSGGGGSGGGGSDIQMTQSPSSLSASVGDRVTITCRASQGIRNDLGW |
| | x I2M2 X | | YQQKPGKAPKRLIYAASTLQSGVPSRFSGSGSGTEFTLTISSLQPEDFATYYCLQHNS |
| | scFc-Full | | YPLTFGCGTKVEIKSGGGGSEVQLLESGGGLVQPGGSLRLSCAASGFTFSHYAMSWVR |
| | Sequence | | QAPGKCLEWVSAVSGGGDKTLYADAVKGRFTISRDNSKNTLFLQMNSLRAEDTAIYYC |
| | | | ARLRGFYYGMDVWGQGTTVTVSSGGGGSGGGGSGGGGSDIVLTQSPLSLPVTPGEPAS |
| | | | ISCRSSQSLLHSNKYNYLDWYLQKPGQSPQLLIYLGSNRASGVPDRFSGSGSGTDFTL |
| | | | KISRVEAEDVGVYYCMQALQTPPITFGCGTRLEIKSGGGGSEVQLVESGGGLVQPGGS |
| | | | LKLSCAASGFTFNKYAINWVREAPGKGLEWVARIRSKYNNYATYYADAVKDRFTISRD |
| | | | DSKNTAYLQMNNLKTEDTAVYYCVRNANFGTSYISYFAYWGQGTLVTVSSGGGGSGGG |
| | | | GSGGGGSQTVVTQEPSLTVSPGGTVTLTCGSSTGAVTSGNYPNWVQKKPGQAPRGLIG |
| | | | GTKFLAPGTPARFSGSLLGGKAALTLSGVQPEDEAEYYCVLWYSNRWVFGSGTKLTVL |
| | | | GGGGDKTHTCPPCPAPELLGGPSVFLFPPKPKDTLMISRTPEVTCVVVDVSHEDPEVK |
| | | | FNWYVDGVEVHNAKTKPCEEQYGSTYRCVSVLTVLHQDWLNGKEYKCKVSNKALPAPI |
| | | | EKTISKAKGQPREPQVYTLPPSREEMTKNQVSLTCLVKGFYPSDIAVEWESNGQPENN |
| | | | YKTTPPVLDSDGSFFLYSKLTVDKSRWQQGNVFSCSVMHEALHNHYTQKSLSLSPGKG |
| | | | GGGSGGGGSGGGGSGGGGSGGGGSGGGGSDKTHTCPPCPAPELLGGPSVFLFPPKPKD |
| | | | TLMISRTPEVTCVVVDVSHEDPEVKFNWYVDGVEVHNAKTKPCEEQYGSTYRCVSVLT |
| | | | VLHQDWLNGKEYKCKVSNKALPAPIEKTISKAKGQPREPQVYTLPPSREEMTKNQVSL |
| | | | TCLVKGFYPSDIAVEWESNGQPENNYKTTPPVLDSDGSFFLYSKLTVDKSRWQQGNVF |
| | | | SCSVMHEALHNHYTQKSLSLSPGK |
| 2529 | CH3 15-E11 | artificial | QVQLVQSGAEVKKPGASVKVSCKASGYTFTNYWMNWVRQAPGQCLEWMGNIAYGVKGT |
| | CC x I2Lopt | | NYNQKFQGRVTMTVDTSSSTAYMELSRLRSDDTAVYYCATRYFYVMDYWGQGTLVTVS |
| | x G4 x scFc | | SGGGGQGGGGQGGGGQDIQMTQSPSSLSASVGDRVTITCRASQDISNYLNWYQQKPGK |
| | x G4 x MS | | VPKLLIYYTSRLHSGVPSRFSGSGSGTDFTLTISSLQPEDVATYYCVQYAQFPLTFGC |
| | 15-B12 CC x | | GTKVEIKSGGGGQEVQLVESGGGLVQPGGSLKLSCAASGFTFNKYAMNWVRQAPGKGM |
| | I2L GQ | | EWVARIRSKYNNYATYYADAVKDRFTISRDDSKNTLYLQMNNLKTEDTAVYYCVRAGN |
| | 27109-1- | | FGSSYISYFAYWGQGTLVTVSSGGGGQGGGGQGGGGQQTVVTQEPSLTVSPGGTVTIT |
| | Full | | CGSSTGAVTSGNYPNWIQKKPGQAPRGLIGGTKFLAPGTPARFSGSLEGGKAALTLSG |
| | Sequence | | VQPEDEAEYYCVLYYSNRWVFGSGTKLTVLGGGGCPPCPAPELLGGPSVFLFPPKPKD |
| | | | TLMISRTPEVTCVVVDVSHEEPEVKENWYVDGVEVHNAKTKPCEEQYGSTYRCVSVLT |
| | | | VLHQDWLNGKEYKCKVSNKALPAPIEKTISKAKGQPREPQVYTLPPSREEMTKNQVSL |
| | | | TCLVKGFYPSDIAVEWESNGQPENNYKTTPPVLDSDGSFFLYSKLTVDKSRWQQGNVF |
| | | | SCSVMHEALHNHYTQKSLSLSPGKGGGGQGGGGQGGGGQGGGGQGGGGQGGGGQCPPC |
| | | | PAPELLGGPSVFLFPPKPKDTLMISRTPEVTCVVVDVSHEEPEVKENWYVDGVEVHNA |
| | | | KTKPCEEQYGSTYRCVSVLTVLHQDWLNGKEYKCKVSNKALPAPIEKTISKAKGQPRE |
| | | | PQVYTLPPSREEMTKNQVSLTCLVKGFYPSDIAVEWESNGQPENNYKTTPPVLDSDGS |
| | | | FFLYSKLTVDKSRWQQGNVFSCSVMHEALHNHYTQKSLSLSPGKGGGGQVQLQESGPG |
| | | | LVKPSETLSLTCTVSGGSISSSSYFWGWIRQPPGKCLEWIGNIYYSGSSNYNPSLKSR |
| | | | VTISVDTSKNQFSLKLSSVTAADTAVYYCARLPRGDRDAFDIWGQGTMVTVSSGGGGQ |
| | | | GGGGQGGGGQDIVMTQSPSSLSASVGDRVTITCRASQGISNYLAWYQQKPGKVPKLLI |
| | | | YAASTLQSGVPSRFSGSGSGTDFTLTISSLQPEDFATYYCQQSYSTPFTFGCGTKVEI |
| | | | KSGGGGQEVQLVESGGGLVQPGGSLKLSCAASGFTFNKYAMNWVRQAPGKGMEWVARI |
| | | | RSKYNNYATYYADAVKDRFTISRDDSKNTLYLQMNNLKTEDTAVYYCVRAGNFGSSYI |
| | | | SYFAYWGQGTLVTVSSGGGGQGGGGQGGGGQQTVVTQEPSLTVSPGGTVTITCGSSTG |
| | | | AVTSGNYPNWIQKKPGQAPRGLIGGTKFLAPGTPARFSGSLEGGKAALTLSGVQPEDE |
| | | | AEYYCVLYYSNRWVFGSGTKLTVL |
| 2530 | CH3 15-E11 | artificial | QVQLVQSGAEVKKPGASVKVSCKASGYTFTNYWMNWVRQAPGQCLEWMGNIAYGVKGT |
| | CC x | | NYNQKFQGRVTMTVDTSSSTAYMELSRLRSDDTAVYYCATRYFYVMDYWGQGTLVTVS |
| | I2M2opt x | | SGGGGQGGGGQGGGGQDIQMTQSPSSLSASVGDRVTITCRASQDISNYLNWYQQKPGK |
| | G4 x scFc x | | VPKLLIYYTSRLHSGVPSRFSGSGSGTDFTLTISSLQPEDVATYYCVQYAQFPLTFGC |
| | G4 x MS 15- | | GTKVEIKSGGGGQEVQLVESGGGLVQPGGSLKLSCAASGFTENKYAINWVREAPGKGL |
| | B12 CC x | | EWVARIRSKYNNYATYYADAVKDRFTISRDDSKNTAYLQMNNLKTEDTAVYYCVRNAN |
| | I2M2_GQ | | FGTSYISYFAYWGQGTLVTVSSGGGGSGGGGSGGGGSQTVVTQEPSLTVSPGGTVTLT |
| | 27109-1- | | CGSSTGAVTSGNYPNWVQKKPGQAPRGLIGGTKFLAPGTPARFSGSLLGGKAALTLSG |
| | Full | | VQPEDEAEYYCVLWYSNRWVFGSGTKLTVLGGGGCPPCPAPELLGGPSVFLFPPKPKD |
| | Sequence | | TLMISRTPEVTCVVVDVSHEEPEVKFNWYVDGVEVHNAKTKPCEEQYGSTYRCVSVLT |
| | | | VLHQDWLNGKEYKCKVSNKALPAPIEKTISKAKGQPREPQVYTLPPSREEMTKNQVSL |
| | | | TCLVKGFYPSDIAVEWESNGQPENNYKTTPPVLDSDGSFFLYSKLTVDKSRWQQGNVF |
| | | | SCSVMHEALHNHYTQKSLSLSPGKGGGGQGGGGQGGGGQGGGGQGGGGQGGGGQCPPC |
| | | | PAPELLGGPSVFLFPPKPKDTLMISRTPEVTCVVVDVSHEEPEVKENWYVDGVEVHNA |
| | | | KTKPCEEQYGSTYRCVSVLTVLHQDWLNGKEYKCKVSNKALPAPIEKTISKAKGQPRE |
| | | | PQVYTLPPSREEMTKNQVSLTCLVKGFYPSDIAVEWESNGQPENNYKTTPPVLDSDGS |
| | | | FFLYSKLTVDKSRWQQGNVFSCSVMHEALHNHYTQKSLSLSPGKGGGGQVQLQESGPG |
| | | | LVKPSETLSLTCTVSGGSISSSSYFWGWIRQPPGKCLEWIGNIYYSGSSNYNPSLKSR |
| | | | VTISVDTSKNQFSLKLSSVTAADTAVYYCARLPRGDRDAFDIWGQGTMVTVSSGGGGQ |
| | | | GGGGQGGGGQDIVMTQSPSSLSASVGDRVTITCRASQGISNYLAWYQQKPGKVPKLLI |
| | | | YAASTLQSGVPSRFSGSGSGTDFTLTISSLQPEDFATYYCQQSYSTPFTFGCGTKVEI |
| | | | KSGGGGQEVQLVESGGGLVQPGGSLKLSCAASGFTFNKYAINWVREAPGKGLEWVARI |
| | | | RSKYNNYATYYADAVKDRFTISRDDSKNTAYLQMNNLKTEDTAVYYCVRNANFGTSYI |
| | | | SYFAYWGQGTLVTVSSGGGGSGGGGSGGGGSQTVVTQEPSLTVSPGGTVTLTCGSSTG |
| | | | AVTSGNYPNWVQKKPGQAPRGLIGGTKFLAPGTPARFSGSLLGGKAALTLSGVQPEDE |
| | | | AEYYCVLWYSNRWVFGSGTKLTVL |
| 2531 | MS R4L CC x | artificial | QVQLVQSGAEVKKPGASVKVSCKASGYTFTGYYIHWVRQAPGQCLEWMGWINPNSGGT |
| | CH3 R170R | | NYAQKFQGRVTMTRDTSISTAYMELSRLRSDDTAVYYCARVEAVAGREYYYFSGMDVW |
| | CC x I2C x | | GQGTTVTVSSGGGGSGGGGSGGGGSSYELTQPPSVSVSPGQTASITCSGEKLGDKYVY |
| | scFc-Full | | WYQQKPGQSPVLVIYQSTKRPSGVPERFSGSNSGNTATLTISGTQAMDEADYYCQAYH |
| | Sequence | | ASTAVFGCGTKLTVLSGGGGSEVQLLESGGGLVQPGGSLRLSCAASGFTFSSYWMHWV |
| | | | RQTPGKCLEWVSKIDPSDDYTNYNQKVKGRFTISIDKSKNTLYLQMNSLRAEDTAVYY |
| | | | CARWDYSHFDVWGQGTTVTVSSGGGGSGGGGSGGGGSEIVMTQSPATLSVSPGERATL |
| | | | TCRASSSVSYMHWYQQKPGQAPRLLIYGTSNLVSGVPARFSGSGSGTEFTLTISSLQS |
| | | | EDFAVYYCQQWSSYPLTFGCGTKVEIKSGGGGSEVQLVESGGGLVQPGGSLKLSCAAS |
| | | | GFTFNKYAMNWVRQAPGKGLEWVARIRSKYNNYATYYADSVKDRFTISRDDSKNTAYL |
| | | | QMNNLKTEDTAVYYCVRHGNFGNSYISYWAYWGQGTLVTVSSGGGGSGGGGSGGGGSQ |
| | | | TVVTQEPSLTVSPGGTVTLTCGSSTGAVTSGNYPNWVQQKPGQAPRGLIGGTKFLAPG |
| | | | TPARFSGSLLGGKAALTLSGVQPEDEAEYYCVLWYSNRWVFGGGTKLTVLGGGGDKTH |
| | | | TCPPCPAPELLGGPSVFLFPPKPKDTLMISRTPEVTCVVVDVSHEDPEVKENWYVDGV |
| | | | EVHNAKTKPCEEQYGSTYRCVSVLTVLHQDWLNGKEYKCKVSNKALPAPIEKTISKAK |
| | | | GQPREPQVYTLPPSREEMTKNQVSLTCLVKGFYPSDIAVEWESNGQPENNYKTTPPVL |
| | | | DSDGSFFLYSKLTVDKSRWQQGNVFSCSVMHEALHNHYTQKSLSLSPGKGGGGSGGGG |
| | | | SGGGGSGGGGSGGGGSGGGGSDKTHTCPPCPAPELLGGPSVFLFPPKPKDTLMISRTP |
| | | | EVTCVVVDVSHEDPEVKFNWYVDGVEVHNAKTKPCEEQYGSTYRCVSVLTVLHQDWLN |
| | | | GKEYKCKVSNKALPAPIEKTISKAKGQPREPQVYTLPPSREEMTKNQVSLTCLVKGFY |
| | | | PSDIAVEWESNGQPENNYKTTPPVLDSDGSFFLYSKLTVDKSRWQQGNVFSCSVMHEA |
| | | | LHNHYTQKSLSLSPGK |
| 2532 | MS R4L CC x | artificial | QVQLVQSGAEVKKPGASVKVSCKASGYTFTGYYIHWVRQAPGQCLEWMGWINPNSGGT |
| | CH3 R170R | | NYAQKFQGRVTMTRDTSISTAYMELSRLRSDDTAVYYCARVEAVAGREYYYFSGMDVW |
| | CC x I2E x | | GQGTTVTVSSGGGGSGGGGSGGGGSSYELTQPPSVSVSPGQTASITCSGEKLGDKYVY |
| | scFc-Full | | WYQQKPGQSPVLVIYQSTKRPSGVPERFSGSNSGNTATLTISGTQAMDEADYYCQAYH |
| | Sequence | | ASTAVFGCGTKLTVLSGGGGSEVQLLESGGGLVQPGGSLRLSCAASGFTFSSYWMHWV |
| | | | RQTPGKCLEWVSKIDPSDDYTNYNQKVKGRFTISIDKSKNTLYLQMNSLRAEDTAVYY |
| | | | CARWDYSHFDVWGQGTTVTVSSGGGGSGGGGSGGGGSEIVMTQSPATLSVSPGERATL |
| | | | TCRASSSVSYMHWYQQKPGQAPRLLIYGTSNLVSGVPARFSGSGSGTEFTLTISSLQS |
| | | | EDFAVYYCQQWSSYPLTFGCGTKVEIKSGGGGSEVQLVESGGGLVQPGGSLKLSCAAS |
| | | | GFTFNKYAINWVRQAPGKGLEWVARIRSKYNNYATYYADAVKDRFTISRDDSKNTVYL |
| | | | QMNNLKTEDTAVYYCARAGNFGSSYISYWAYWGQGTLVTVSSGGGGSGGGGSGGGGSQ |
| | | | TVVTQEPSLTVSPGGTVTITCGSSTGAVTSGNYPNWVQKKPGQAPRGLIGGTKFLAPG |
| | | | TPARFSGSLSGGKAALTLSGVQPEDEAEYYCVLWYSNRWVFGSGTKLTVLGGGGDKTH |
| | | | TCPPCPAPELLGGPSVFLFPPKPKDTLMISRTPEVTCVVVDVSHEDPEVKENWYVDGV |
| | | | EVHNAKTKPCEEQYGSTYRCVSVLTVLHQDWLNGKEYKCKVSNKALPAPIEKTISKAK |
| | | | GQPREPQVYTLPPSREEMTKNQVSLTCLVKGFYPSDIAVEWESNGQPENNYKTTPPVL |
| | | | DSDGSFFLYSKLTVDKSRWQQGNVFSCSVMHEALHNHYTQKSLSLSPGKGGGGSGGGG |
| | | | SGGGGSGGGGSGGGGSGGGGSDKTHTCPPCPAPELLGGPSVFLFPPKPKDTLMISRTP |
| | | | EVTCVVVDVSHEDPEVKFNWYVDGVEVHNAKTKPCEEQYGSTYRCVSVLTVLHQDWLN |
| | | | GKEYKCKVSNKALPAPIEKTISKAKGQPREPQVYTLPPSREEMTKNQVSLTCLVKGFY |
| | | | PSDIAVEWESNGQPENNYKTTPPVLDSDGSFFLYSKLTVDKSRWQQGNVFSCSVMHEA |
| | | | LHNHYTQKSLSLSPGK |
| 2533 | MS R4L CC x | artificial | QVQLVQSGAEVKKPGASVKVSCKASGYTFTGYYIHWVRQAPGQCLEWMGWINPNSGGT |
| | CH3 R170R | | NYAQKFQGRVTMTRDTSISTAYMELSRLRSDDTAVYYCARVEAVAGREYYYFSGMDVW |
| | CC x I2K x | | GQGTTVTVSSGGGGSGGGGSGGGGSSYELTQPPSVSVSPGQTASITCSGEKLGDKYVY |
| | scFc-Full | | WYQQKPGQSPVLVIYQSTKRPSGVPERFSGSNSGNTATLTISGTQAMDEADYYCQAYH |
| | Sequence | | ASTAVFGCGTKLTVLSGGGGSEVQLLESGGGLVQPGGSLRLSCAASGFTFSSYWMHWV |
| | | | RQTPGKCLEWVSKIDPSDDYTNYNQKVKGRFTISIDKSKNTLYLQMNSLRAEDTAVYY |
| | | | CARWDYSHFDVWGQGTTVTVSSGGGGSGGGGSGGGGSEIVMTQSPATLSVSPGERATL |
| | | | TCRASSSVSYMHWYQQKPGQAPRLLIYGTSNLVSGVPARFSGSGSGTEFTLTISSLQS |
| | | | EDFAVYYCQQWSSYPLTFGCGTKVEIKSGGGGSEVQLVESGGGLVQPGGSLKLSCAAS |
| | | | GFTFNKYAMNWVRQAPGKGLEWVARIRSKYNNYATYYAEAVKGRFTISRDDSKNTVYL |
| | | | QMNNLKTEDTAVYYCVRNENIGTSYISYWAYWGQGTLVTVSSGGGGSGGGGSGGGGSQ |
| | | | TVVTQEPSLTVSPGGTVTITCGSSTGAVTSGNYPNWVQQKPGQAPRGLIGGTKFLAPG |
| | | | TPARFSGSLSGGKAALTLSGVQPEDEAEYYCVLWYSNRWVFGSGTKLTVLGGGGDKTH |
| | | | TCPPCPAPELLGGPSVFLFPPKPKDTLMISRTPEVTCVVVDVSHEDPEVKENWYVDGV |
| | | | EVHNAKTKPCEEQYGSTYRCVSVLTVLHQDWLNGKEYKCKVSNKALPAPIEKTISKAK |
| | | | GQPREPQVYTLPPSREEMTKNQVSLTCLVKGFYPSDIAVEWESNGQPENNYKTTPPVL |
| | | | DSDGSFFLYSKLTVDKSRWQQGNVFSCSVMHEALHNHYTQKSLSLSPGKGGGGSGGGG |
| | | | SGGGGSGGGGSGGGGSGGGGSDKTHTCPPCPAPELLGGPSVFLFPPKPKDTLMISRTP |
| | | | EVTCVVVDVSHEDPEVKFNWYVDGVEVHNAKTKPCEEQYGSTYRCVSVLTVLHQDWLN |
| | | | GKEYKCKVSNKALPAPIEKTISKAKGQPREPQVYTLPPSREEMTKNQVSLTCLVKGFY |
| | | | PSDIAVEWESNGQPENNYKTTPPVLDSDGSFFLYSKLTVDKSRWQQGNVFSCSVMHEA |
| | | | LHNHYTQKSLSLSPGK |
| 2534 | MS R4L CC x | artificial | QVQLVQSGAEVKKPGASVKVSCKASGYTFTGYYIHWVRQAPGQCLEWMGWINPNSGGT |
| | CH3 R170R | | NYAQKFQGRVTMTRDTSISTAYMELSRLRSDDTAVYYCARVEAVAGREYYYFSGMDVW |
| | CC x I2L x | | GQGTTVTVSSGGGGSGGGGSGGGGSSYELTQPPSVSVSPGQTASITCSGEKLGDKYVY |
| | scFc-Full | | WYQQKPGQSPVLVIYQSTKRPSGVPERFSGSNSGNTATLTISGTQAMDEADYYCQAYH |
| | Sequence | | ASTAVFGCGTKLTVLSGGGGSEVQLLESGGGLVQPGGSLRLSCAASGFTFSSYWMHWV |
| | | | RQTPGKCLEWVSKIDPSDDYTNYNQKVKGRFTISIDKSKNTLYLQMNSLRAEDTAVYY |
| | | | CARWDYSHFDVWGQGTTVTVSSGGGGSGGGGSGGGGSEIVMTQSPATLSVSPGERATL |
| | | | TCRASSSVSYMHWYQQKPGQAPRLLIYGTSNLVSGVPARFSGSGSGTEFTLTISSLQS |
| | | | EDFAVYYCQQWSSYPLTFGCGTKVEIKSGGGGSEVQLVESGGGLVQPGGSLKLSCAAS |
| | | | GFTFNKYAMNWVRQAPGKGMEWVARIRSKYNNYATYYADAVKDRFTISRDDSKNTLYL |
| | | | QMNNLKTEDTAVYYCVRAGNFGSSYISYFAYWGQGTLVTVSSGGGGSGGGGSGGGGSQ |
| | | | TVVTQEPSLTVSPGGTVTITCGSSTGAVTSGNYPNWIQKKPGQAPRGLIGGTKFLAPG |
| | | | TPARFSGSLEGGKAALTLSGVQPEDEAEYYCVLYYSNRWVFGSGTKLTVLGGGGDKTH |
| | | | TCPPCPAPELLGGPSVFLFPPKPKDTLMISRTPEVTCVVVDVSHEDPEVKENWYVDGV |
| | | | EVHNAKTKPCEEQYGSTYRCVSVLTVLHQDWLNGKEYKCKVSNKALPAPIEKTISKAK |
| | | | GQPREPQVYTLPPSREEMTKNQVSLTCLVKGFYPSDIAVEWESNGQPENNYKTTPPVL |
| | | | DSDGSFFLYSKLTVDKSRWQQGNVFSCSVMHEALHNHYTQKSLSLSPGKGGGGSGGGG |
| | | | SGGGGSGGGGSGGGGSGGGGSDKTHTCPPCPAPELLGGPSVFLFPPKPKDTLMISRTP |
| | | | EVTCVVVDVSHEDPEVKFNWYVDGVEVHNAKTKPCEEQYGSTYRCVSVLTVLHQDWLN |
| | | | GKEYKCKVSNKALPAPIEKTISKAKGQPREPQVYTLPPSREEMTKNQVSLTCLVKGFY |
| | | | PSDIAVEWESNGQPENNYKTTPPVLDSDGSFFLYSKLTVDKSRWQQGNVFSCSVMHEA |
| | | | LHNHYTQKSLSLSPGK |
| 2535 | MS R4L CC x | artificial | QVQLVQSGAEVKKPGASVKVSCKASGYTFTGYYIHWVRQAPGQCLEWMGWINPNSGGT |
| | CH3 R170R | | NYAQKFQGRVTMTRDTSISTAYMELSRLRSDDTAVYYCARVEAVAGREYYYFSGMDVW |
| | CC x I2M x | | GQGTTVTVSSGGGGSGGGGSGGGGSSYELTQPPSVSVSPGQTASITCSGEKLGDKYVY |
| | scFc-Full | | WYQQKPGQSPVLVIYQSTKRPSGVPERFSGSNSGNTATLTISGTQAMDEADYYCQAYH |
| | Sequence | | ASTAVFGCGTKLTVLSGGGGSEVQLLESGGGLVQPGGSLRLSCAASGFTFSSYWMHWV |
| | | | RQTPGKCLEWVSKIDPSDDYTNYNQKVKGRFTISIDKSKNTLYLQMNSLRAEDTAVYY |
| | | | CARWDYSHFDVWGQGTTVTVSSGGGGSGGGGSGGGGSEIVMTQSPATLSVSPGERATL |
| | | | TCRASSSVSYMHWYQQKPGQAPRLLIYGTSNLVSGVPARFSGSGSGTEFTLTISSLQS |
| | | | EDFAVYYCQQWSSYPLTFGCGTKVEIKSGGGGSEVQLVESGGGLVQPGGSLKLSCAAS |
| | | | GFTFNKYAMNWVRQAPGKGMEWVARIRSKYNNYATYYADAVKDRFTISRDDSKNTLYL |
| | | | QMNNLKTEDTAVYYCVRAGNFGTSYISYWAYWGQGTLVTVSSGGGGSGGGGSGGGGSQ |
| | | | TVVTQEPSLTVSPGGTVTITCGSSTGAVTSGNYPNWVQKKPGQAPRGLIGGTKFLAPG |
| | | | TPARFSGSLSGGKAALTLSGVQPEDEAEYYCVLWYSNRWVFGSGTKLTVLGGGGDKTH |
| | | | TCPPCPAPELLGGPSVFLFPPKPKDTLMISRTPEVTCVVVDVSHEDPEVKENWYVDGV |
| | | | EVHNAKTKPCEEQYGSTYRCVSVLTVLHQDWLNGKEYKCKVSNKALPAPIEKTISKAK |
| | | | GQPREPQVYTLPPSREEMTKNQVSLTCLVKGFYPSDIAVEWESNGQPENNYKTTPPVL |
| | | | DSDGSFFLYSKLTVDKSRWQQGNVFSCSVMHEALHNHYTQKSLSLSPGKGGGGSGGGG |
| | | | SGGGGSGGGGSGGGGSGGGGSDKTHTCPPCPAPELLGGPSVFLFPPKPKDTLMISRTP |
| | | | EVTCVVVDVSHEDPEVKFNWYVDGVEVHNAKTKPCEEQYGSTYRCVSVLTVLHQDWLN |
| | | | GKEYKCKVSNKALPAPIEKTISKAKGQPREPQVYTLPPSREEMTKNQVSLTCLVKGFY |
| | | | PSDIAVEWESNGQPENNYKTTPPVLDSDGSFFLYSKLTVDKSRWQQGNVFSCSVMHEA |
| | | | LHNHYTQKSLSLSPGK |
| 2536 | MS R4L CC x | artificial | QVQLVQSGAEVKKPGASVKVSCKASGYTFTGYYIHWVRQAPGQCLEWMGWINPNSGGT |
| | CH3 R170R | | NYAQKFQGRVTMTRDTSISTAYMELSRLRSDDTAVYYCARVEAVAGREYYYFSGMDVW |
| | CC x I2M2 x | | GQGTTVTVSSGGGGSGGGGSGGGGSSYELTQPPSVSVSPGQTASITCSGEKLGDKYVY |
| | scFc-Full | | WYQQKPGQSPVLVIYQSTKRPSGVPERFSGSNSGNTATLTISGTQAMDEADYYCQAYH |
| | Sequence | | ASTAVFGCGTKLTVLSGGGGSEVQLLESGGGLVQPGGSLRLSCAASGFTFSSYWMHWV |
| | | | RQTPGKCLEWVSKIDPSDDYTNYNQKVKGRFTISIDKSKNTLYLQMNSLRAEDTAVYY |
| | | | CARWDYSHFDVWGQGTTVTVSSGGGGSGGGGSGGGGSEIVMTQSPATLSVSPGERATL |
| | | | TCRASSSVSYMHWYQQKPGQAPRLLIYGTSNLVSGVPARFSGSGSGTEFTLTISSLQS |
| | | | EDFAVYYCQQWSSYPLTFGCGTKVEIKSGGGGSEVQLVESGGGLVQPGGSLKLSCAAS |
| | | | GFTFNKYAINWVREAPGKGLEWVARIRSKYNNYATYYADAVKDRFTISRDDSKNTAYL |
| | | | QMNNLKTEDTAVYYCVRNANFGTSYISYFAYWGQGTLVTVSSGGGGSGGGGSGGGGSQ |
| | | | TVVTQEPSLTVSPGGTVTLTCGSSTGAVTSGNYPNWVQKKPGQAPRGLIGGTKFLAPG |
| | | | TPARFSGSLLGGKAALTLSGVQPEDEAEYYCVLWYSNRWVFGSGTKLTVLGGGGDKTH |
| | | | TCPPCPAPELLGGPSVFLFPPKPKDTLMISRTPEVTCVVVDVSHEDPEVKENWYVDGV |
| | | | EVHNAKTKPCEEQYGSTYRCVSVLTVLHQDWLNGKEYKCKVSNKALPAPIEKTISKAK |
| | | | GQPREPQVYTLPPSREEMTKNQVSLTCLVKGFYPSDIAVEWESNGQPENNYKTTPPVL |
| | | | DSDGSFFLYSKLTVDKSRWQQGNVFSCSVMHEALHNHYTQKSLSLSPGKGGGGSGGGG |
| | | | SGGGGSGGGGSGGGGSGGGGSDKTHTCPPCPAPELLGGPSVFLFPPKPKDTLMISRTP |
| | | | EVTCVVVDVSHEDPEVKFNWYVDGVEVHNAKTKPCEEQYGSTYRCVSVLTVLHQDWLN |
| | | | GKEYKCKVSNKALPAPIEKTISKAKGQPREPQVYTLPPSREEMTKNQVSLTCLVKGFY |
| | | | PSDIAVEWESNGQPENNYKTTPPVLDSDGSFFLYSKLTVDKSRWQQGNVFSCSVMHEA |
| | | | LHNHYTQKSLSLSPGK |
| 2551 | (G4Q)3- | artificial | GGGGQGGGGQGGGGQ |
| | Linker | | |
| 2552 | Human | artificial | QDGNEEMGGITQTPYKVSISGTTVILTCPQYPGSEILWQHNDKNIGGDEDDKNIGSDE |
| | CD3epsilon | | DHLSLKEFSELEQSGYYVCYPRGSKPEDANFYLYLRARVCENCMEMD |
| | ECD | | |
| 2553 | Human | artificial | QDGNEEMGGITQTPYKVSISGTTVILT |
| | CD3epsilon | | |
| | ECD/pos. | | |
| | 1-27 | | |
| 2554 | I2C_HC_H109A- | artificial | KYAMN |
| | HCDR1 | | |
| 2555 | I2C_HC_H109A- | artificial | RIRSKYNNYATYYADSVKD |
| | HCDR2 | | |
| 2556 | I2C_HC_H109A- | artificial | AGNFGNSYISYWAY |
| | HCDR3 | | |
| 2557 | I2C_HC_H109A- | artificial | GSSTGAVTSGNYPN |
| | LCDR1 | | |
| 2558 | I2C_HC_H109A- | artificial | GTKFLAP |
| | LCDR2 | | |
| 2559 | I2C_HC_H109A- | artificial | VLWYSNRWV |
| | LCDR3 | | |
| 2560 | I2C_HC_H109A- | artificial | EVQLVESGGGLVQPGGSLKLSCAASGFTFNKYAMNWVRQAPGKGLEWVARIRSKYNNY |
| | VH | | ATYYADSVKDRFTISRDDSKNTAYLQMNNLKTEDTAVYYCVRAGNFGNSYISYWAYWG |
| | | | QGTLVTVSS |
| 2561 | I2C_HC_H109A- | artificial | QTVVTQEPSLTVSPGGTVTLTCGSSTGAVTSGNYPNWVQQKPGQAPRGLIGGTKFLAP |
| | VL | | GTPARFSGSLLGGKAALTLSGVQPEDEAEYYCVLWYSNRWVFGGGTKLTVL |

1. A polypeptide or polypeptide construct comprising:
a binding domain binding to an extracellular epitope of the human CD3ε chain comprising or consisting of a VH region and a VL region, wherein
i) the VH region comprises:
a CDR-H1 sequence of X₁YAX₂N, where X, is K, V, S, G, R, T, or I; and X₂ is M or I;
a CDR-H2 sequence of RIRSKYNNYATYYADX₁VKX₂, where X, is S or Q; and X₂ is D, G, K, S, or E; and
a CDR-H3 sequence of HX₁NFGNSYX₂SX₃X₄AY, where X, is G, R, or A; X₂ is I, L, V, or T; X₃ is Y, W or F; and X₄ is W, F or Y; and
ii) wherein the VL region comprises:
a CDR-L1 sequence of X₁SSTGAVTX₂X₃X₄YX₅N, where X, is G, R, or A; X₂ is S or T; X₃ is G or S; X₄ is N or Y; and X₅ is P or A;
a CDR-L2 sequence of X₁TX₂X₃X₄X₅X₆; where X, is G or A; X₂ is K, D, or N; X₃ is F, M or K; X₄ is L or R; X₅ is A, P, or V; and X₆ is P or S; and
a CDR-L3 sequence of X₁LWYSNX₂WV, where X, is V, A, or T; and X₂ is R or L; and
iii) wherein one or more of CDR sequences of the VH region of i) and/or of the VL region of ii) comprise one amino acid substitution or a combination thereof selected from X₂4V and X₂4F in CDR-H1;
D15, and X₁16A in CDR-H2;
H1, X₁2E, F4, and N6 in CDR-H3; and
X₁1L and W3 in CDR-L3.

2. The polypeptide or polypeptide construct of claim 1, wherein in addition to said one amino acid substitution or a combination thereof as defined in iii) X₂ is I in said CDR-H1 sequence; X₂ is G in said CDR-H2 sequence; X₁ is A, X₄ is F in said CDR-H3 sequence; and/or X₁ is A in said CDR-L3 sequence.

3. The polypeptide or polypeptide construct of claim 1 or 2, comprising combinations of said amino acid substitutions as defined in iii) of two, three, four, five, six, seven, eight, nine, ten, eleven, twelve or more amino acid substitutions.

4. The polypeptide or polypeptide construct of any one of claims 1 to 3, wherein:
i) said one amino acid substitution is selected from:
a. X₂4V or X₂4F in CDR-H1; H1A in CDR-H3; and
b. X₁1L and W3Y in CDR-L3;
ii) said combination of two or more amino acid substitutions are selected from X₁16A in CDR-H2 and N6S in CDR-H3; and X₁16A in CDR-H2 and N6T in CDR-H3;
iii) said combination of three or more amino acid substitutions are selected from X₁16A in CDR-H2, H1A and N6S in CDR-H3; X₁16A in CDR-H2, H1A and N6T in CDR-H3; X₁16A in CDR-H2, H1N and N6T in CDR-H3;
iv) said combination of four or more amino acid substitutions are selected from X₁16A in CDR-H2, H1A, N6S in CDR-H3, and W3Y in CDR-L3; D15E, X₁16A in CDR-H2, H1A, and N6T in CDR-H3; D15E, X₁16A in CDR-H2, H1A, and N6S in CDR-H3; X₁16A in CDR-H2, H1A, F41 and N6S in CDR-H3; or
v) said combination of five or six amino acid substitutions are selected from X₁16A in CDR-H2, H1A, X₁2E, F41, and N6T in CDR-H3; X₁16A in CDR-H2, H1N, X₁2E, F41, and N6S in CDR-H3; D15E, X₁16A in CDR-H2, H1A, N6S in CDR-H3; and W3Y in CDR-L3; D15E, X₁16A in CDR-H2, H1N, X₁2E, F41 and N6T in CDR-H3; D15E, X₁16A in CDR-H2, H1A, X₁2E, F41 and N6T in CDR-H3; D15E, X₁16A in CDR-H2, X₁2E, F41 and N6T in CDR-H3.

5. A polypeptide or polypeptide construct comprising: a binding domain binding to an extracellular epitope of the human CD3ε chain comprising or consisting of a VH region and a VL region, wherein
i) the VH region comprises the sequence of EVX₁LX₂ESGGGLX₃QPX₄GSLKLSCAASGFTFNX₅YAX₆NWVRQAPGKGLEWVAR IRSKYNNYATYYADX₇VKX₈RFTISRDDSXX₁₀X₁₁X₁₂YLQMNNLKTEDTAX₁₃YYCV RHX₁₄NFGNSYX₁₅SX₁₆X₁₇AYWGQGTLVTVSX₁₈, where X₁ is Q or K; X₂ is V or L; X₃ is V or E; X₄ is G or K; X₅ is K, V, S, G, R, T, or I; X₆ is M or I; X₇ is S or Q; X₈ is D, G, K, S, or E; X₉ is K or Q; X₁₀ is N or S; X₁₁ is T or I; X₁₂ is A or L; X₁₃ is V or M; X₁₄ is G, R or A; X₁₅ is I, L, V, or T; X₁₆ is Y, W or F; X₁₇ is W, F or Y; and X₁₈ is S or A; and
ii) the VL region comprises the sequence of QX₁VVTQEX₂X₃LTX₄SPGX₅TVTLTCX₆SSTGAVTX₇X₈X₉YX₁₀NWVQX₁₁KPX₁₂X₁₃X₁₄X₁₅X₁₆GLIGX₁₇TX₁₈X₁₉X₂₀X₂₁X₂₂GX₂₃PARFSGSLX₂₄GX₂₅KAALTX₂₆X₂₇GX₂₈QX₂₉E DEAX₃₀YX₃₁CX₃₂LWYSNX₃₃WVFGGGTKLTVL, where X₁ is T or A; X₂ is P or S; X₃ is S or A; X₄ is V or T; X₅ is G or E; X₆ is G, R, or A; X₇ is S or T; X₈ is G or S; X₉ is N or Y; X₁₀ is P or A; X₁₁ is Q or E; X₁₂ is G or D; X₁₃ is Q or H; X₁₄ is A or L; X₁₅ is P or F; X₁₆ is R or T; X₁₇ is G or A; X₁₈ is K or D; X₁₉ is F or M; X₂₀ is L or R; X₂₁ is A, P or V; X₂₂ is P or S; X₂₃ is T or V; X₂₄ is L or I; X₂₅ is G or D; X₂₆ is L or I; X₂₇ is S or T; X₂₈ is V or A; X₂₉ is P or T; X₃₀ is E or I; X₃₁ is Y or F; X₃₂ is V, A, or T; X₃₃ is R or L; and
iii) wherein the VH and/or VL region sequence comprises one amino acid substitution or a combination thereof selected from
a. N30, X₆34V, X₆34F, Q39, L45, D64, X₇65A, X₁₂81V, X₁₂81T, X₁₂811, V99, H101, X₁₄102E, F104, and N106 in the VH region sequence in i); and
b. L20, V38, X₁₁40R, X₁₁40K, X₂₄69, X₃₂91L, X₃₃93, and G102 in the VL region sequence in ii).

6. The polypeptide or polypeptide construct of claim 5, wherein in addition to said one amino acid substitution or a combination thereof as defined in iii) X₆ is I; X₈ is G; X₁₂ is L, X₁₄ is A, X₁₇ is F in said VH region sequence; and/or X₃₂ is A in said VL region sequence.

7. The polypeptide or polypeptide construct of claim 5 or 6, comprising combinations of said amino acid substitutions as defined in iii) of two, three, four, five, six, seven, eight, nine, ten, eleven, twelve or more amino acid substitutions.

8. The polypeptide or polypeptide construct of any one of claims 5 to 7, wherein:
i) said one acid substitution is selected from
a. X₆34V, Q39E, Q39K, Q39R, Q39D, L45M, L45V, X₁₂81V, X₁₂81T, X₁₂811, V99A, V99L, H101A, H101N in the VH region; and
b. V381, V38L, V38M, V38F, V38Y, X₁₁40R, X₁₁40K, X₂₄69S, X₂₄69E, X₃₂91L, X₃₃93Y;
ii) said combination of two or more amino acid substitutions are selected from X₆34F and X₁₂81V in the VH region; Q39E in the VH region and X₁₁40K in the VL region;
Q39E in the VH region and X₁₁40R in the VL region; Q39D in the VH region and X₁₁40K in the VL region; Q39D in the VH region and X₁₁40R in the VL region; L45V in the VH region and V38F in the VL region; L45V in the VH region and V38Y in the VL region; L45M in the VH region and X₁₁40K in the VL region; L20I and G102S in the VL region; X₁₂81V in the VH region and X₁₁40K in the VL region;
iii) said combination of three or more amino acid substitutions are selected from Q39E in the VH region, X₁₁40K and G102S in the VL region; Q39E in the VH region, X₁₁40R and G102S in the VL region; Q39D in the VH region, X₁₁40K and G102S in the VL region; Q39D in the VH region, X₁₁40R and G102S in the VL region; L45M in the VH region, X₁₁40K and G102S in the VL region; X₇65A, H101A, and N106S in the VH region; L20I, X₁₁40K, and G102S in the VL region;
iv) said combination of four or more amino acid substitutions are selected from Q39E in the VH region, L20I, X₁₁40K and G102S in the VL region; Q39E in the VH region, L20I, X₁₁40R and G102S in the VL region; Q39D in the VH region, L20I, X₁₁40K and G102S in the VL region; Q39D in the VH region, L20I, X₁₁40R and G102S in the VL region; L45M in the VH region, L20I, X₁₁40K and G102S in the VL region; L20I, X₁₁40K, X₂₄69S, and G102S in the VL region; X₁₂81V in the VH region, L20I, X₁₁40K, and G102S in the VL region;
v) said combination of five, six, seven, eight, nine, ten, or more amino acid substitutions are selected from X₇65A, X₁₂81V, V99A, N106S in the VH region, L20I, X₁₁40K, X₂₄69S, and G102S in the VL region; D64E, X₇65A, X₁₂81V, X₁₄102E, F104I, N106T in the VH region, L20I, X₂₄69S, and G102S in the VL region; L45M, X₇65A, N106T in the VH region, L20I, X₁₁40K, X₂₄69S, and G102S in the VL region; X₇65A, X₁₂81V, V99A, H101A, F104I, N106S in the VH region, L20I, X₁₁40K, X₂₄69S, and G102S in the VL region; X₇65A, X₁₂81V, V99A, H101A, N106S in the VH region; X₁₂81V, V99A in the VH region, L20I, X₁₁40K, X₂₄69S, and G102S in the VL region; Q39E, X₁₂81V in the VH region, L20I, X₁₁40K, and G102S in the VL region; L45V in the VH region, L20I, V38F, X₁₁40K and G102S in the VL region.

9. The polypeptide or polypeptide construct of any one of claims 5 to 8, wherein said combination of amino acid substitutions is selected from:
ii) in the VH region
a. X₇65A, X₁₂81V, V99A, H101A and N106S;
b. D64E, X₇65A, A81V, H101N, X₁₄102E, F104I, and N106S;
c. L45M, X₇65A, H101A, and N106T;
d. L45M, X₇65A, H101A, and N106S;
e. Q39E, X₇65A, H101N, and N106T;
f. D64E, X₇65A, V99A, H101A, and N106T;
g. X₇65A, X₁₂81V, V99A, H101A, X₁₄102E, F104I, and N106T;
h. X₇65A, X₁₂81V, H101N, X₁₄102E, F104I, and N106S;
i. D64E, X₇65A, X₁₂81V, H101A, and N106S;
j. D64E, X₇65A, H101A, and N106T;
k. X₇65A, V99A, H101A, and N106T;
l. D64E, X₇65A, H101A, and N106S;
m. D64E, X₇65A, X₁₂81V, V99A, H101A, and N106S;
n. X₇65A, H101A and N106S;
o. N30S, Q39E, D64E, X₇65A, X₁₂81V, H101A, X₁₄102E, F104I, and N106T;
p. L45M, D64E, X₇65A, H101A, and N106T;
q. N30S, L45M, X₇65A, X₁₂81V, H101A, and N106T;
r. N30S, L45M, D64E, X₇65A, X₁₂81V, H101A, and N106S;
iii) in the VL region
a. L20I, X₁₁40K, X₂₄69S, and G102S;
b. L20I, X₂₄69S, and G102S;
c. L20I, V381, X₁₁40K, X₂₄69E, G102S and X₃₃93Y;
d. X₁₁40K and G102S;
e. L20I, X₁₁40K, X₂₄69S, G102S and X₃₃93Y;
f. L20M, X₁₁40K and X₂₄69E;
g. L20I, V381, X₁₁40K, X₂₄69E and G102S;
h. X₁₁40K, X₂₄69S and X₃₃93Y; or
i. X₁₁40K and X₂₄69S; and
iv) a combination of one amino acid substitution combination of i) and ii).

10. The polypeptide or polypeptide construct of claim 9, wherein said combination of amino acid substitution combinations of iii) is selected from:
a. X₇65A, X₁₂81V, V99A, H101A, N106S in the VH region, L20I, X₁₁40K, X₂₄69S, and G102S in the VL region;
b. D64E, X₇65A, X₁₂81V, H101N, X₁₄102E, F104I, N106S in the VH region, L20I, X₂₄69S, and G102S in the VL region:
c. L45M, X₇65A, H101A, N106T in the VH region, L20I, X₁₁40K, X₂₄69S, and G102S in the VL region;
d. L45M, X₇65A, H101A, N106S in the VH region, L20I, V381, X₁₁40K, X₂₄69E, G102S and X₃₃93Y in the VL region;
e. Q39E, X₇65A, H101N, N106T in the VH region, X₁₁40K and G102S in the VL region;
f. D64E, X₇65A, V99A, H101A, N106T in the VH region, X₁₁40K and G102S in the VL region;
g. X₇65A, X₁₂81V, V99A, H101A, X₁₄102E, F104I, N106T in the VH region, L20I, X₁₁40K, X₂₄69S, and G102S in the VL region;
h. X₇65A, X₁₂81V, H101N, X₁₄102E, F104I, N106S in the VH region, L20I, X₂₄69S, and G102S in the VL region;
i. D64E, X₇65A, X₁₂81V, H101A, N106S in the VH region, L20I, X₂₄69S, and G102S in the VL region;
j. D64E, X₇65A, H101A, N106T in the VH region, X₁₁40K and G102S in the VL region;
k. X₇65A, V99A, H101A, N106T in the VH region, X₁₁40K and G102S in the VL region;
l. D64E, X₇65A, H101A, N106S in the VH region, X₁₁40K and G102S in the VL region;
m. D64E, X₇65A, X₁₂81V, V99A, H101A, N106S in the VH region, L20I, X₁₁40K, X₂₄69S, G102S and X₃₃93Y in the VL region;
n. X₇65A, H101A, N106S in the VH region, X₁₁40K and G102S in the VL region;
o. N30S, Q39E, D64E, X₇65A, X₁₂81V, H101A, X₁₄102E, F104I, N106T in the VH region, L20M, X₁₁40K and X₂₄69E in the VL region;
p. L45M, D64E, X₇65A, H101A, N106T in the VH region, L20I, V381, X₁₁40K, X₂₄69E and G102S in the VL region;
q. N30S, L45M, X₇65A, X₁₂81V, H101A, N106T in the VH region, X₁₁40K and X₂₄69S in the VL region; and
r. N30S, L45M, D64E, X₇65A, X₁₂81V, H101A, N106S in the VH region, X₁₁40K and X₂₄69S in the VL region.

11. The polypeptide or polypeptide construct of claim 10, wherein in said combination of amino acid substitution combinations defined in:
a. and m. X₆ is I in said VH region sequence;
b. and r. X₃ is G in said VH region sequence;
c. X₁₂ is L in said VH region;
d. X₁₂ is L and X₁₇ is F in said VH region;
e. X₆ is I, X₁₄ is A and X₁₇ is F in said VH region sequence;
f. X₅ is I in said VH region sequence; and X₃₂ is A in said VL region sequence;
g. X₅ is I in said VH region sequence;
h. and i. X₃ is G in said VH region sequence;
j., l. and n. X₆ is I and X₁₇ is F in said VH region sequence; and X₃₂ is A in said VL region sequence;
k. X₆ is I said VH region sequence; and X₃₂ is A in said VL region sequence;
p. X₁₂ is L in said VH region; and
q. X₃ is G and X₁₇ is F in said VH region sequence.

12. The polypeptide or polypeptide construct of claim 5, or any one of claims 6 to 11, wherein said VH and VL region of i) and ii) is selected from the VH and VL region combinations as defined in SEQ ID NOs: 124 and 125, 172 and 173, 362 and 363, 552 and 553, 730 and 731, 928 and 929, 1106 and 1107, 1296 and 1297, 1474 and 1475, 1664 and 1665, and 1854 and 1855, wherein said one amino acid substitution or a combination thereof in said VH and/or VL region sequence results in a VH and/or a VL region sequence having the amino acid residue:
i. I at position 34, A at position 65, V at position 81, A at position 99, A at position 101, S at position 106 in the VH region sequence, I at position 20, K at position 40, S at position 69, and S at position 102 in the VL region sequence;
M at position 45, A at position 65, L at position 81, A at position 101, T at position 106 in the VH region sequence, I at position 20, K at position 40, S at position 69 and S at position 102 in the VL region sequence;
M at position 45, A at position 65, L at position 81, A at position 101, S at position 106, F at position 112 in the VH region sequence, I at position 20, 1 at position 38, K at position 40, E at position 69, S at position 102 and Y at position 93 in the VL region sequence;
I at position 34, E at position 39, A at position 65, N at position 101, A at position 102, T at position 106, F at position 112 in the VH region sequence, K at position 40 and S at position 102 in the VL region sequence;
I at position 34, E at position 64, A at position 65, A at position 99, A at position 101, T at position 106 in the VH region sequence, K at position 40, A at position 91 and S at position 102 in the VL region sequence;
I at position 34, A at position 65, V at position 81, A at position 99, A at position 101, E at position 102, I at position 104, T at position 106 in the VH region sequence, I at position 20, K at position 40, S at position 69 and S at position 102 in the VL region sequence;
E at position 64, A at position 65, G at position 68, V at position 81, A at position 101, S at position 106 in the VH region sequence, I at position 20, S at position 69 and S at position 102 in the VL region sequence;
I at position 34, E at position 64, A at position 65, A at position 101, T at position 106, F at position 112 in the VH region sequence, K at position 40, A at position 91 and S at position 102 in the VL region sequence;
I at position 34, E at position 64, A at position 65, A at position 101, S at position 106, F at position 112 in the VH region sequence, K at position 40, A at position 91 and S at position 102 in the VL region sequence;
I at position 34, E at position 64, A at position 65, V at position 81, A at position 99, A at position 101, S at position 106 in the VH region sequence, I at position 20, K at position 40, S at position 69, S at position 102 and Y at position 93 in the VL region sequence;
I at position 34, A at position 65, A at position 101, S at position 106, F at position 112 in the VH region sequence, K at position 40, A at position 91 and S at position 102 in the VL region sequence;
M at position 45, E at position 64, A at position 65, L at position 81, A at position 101, T at position 106 in the VH region sequence, I at position 20, 1 at position 38, K at position 40, E at position 69 and S at position 102 in the VL region sequence;
S at position 30, M at position 45, A at position 65, G at position 68, V at position 81, A at position 101, T at position 106, F at position 112 in the VH region sequence, K at position 40, S at position 69 and Y at position 93 in the VL region sequence; and
S at position 30, M at position 45, E at position 64, A at position 65, G at position 68, V at position 81, A at position 101, S at position 106 in the VH region sequence, K at position 40 and S at position 69 in the VL region sequence;
ii. E at position 64, A at position 65, G at position 68, V at position 81, N at position 101, E at position 102, I at position 104, T at position 106 in the VH region sequence, I at position 20, S at position 69 and S at position 102 in the VL region sequence;
A at position 65, G at position 68, V at position 81, N at position 101, E at position 102, I at position 104, S at position 106 in the VH region sequence, I at position 20, S at position 69 and S at position 102 in the VL region sequence;
I at position 34, A at position 65, A at position 99, A at position 101, T at position 106 in the VH region sequence, K at position 40, A at position 91 and S at position 102 in the VL region sequence; and
S at position 30, E at position 39, E at position 64, A at position 65, V at position 81, A at position 101, E at position 102, 1 at position 104, T at position 106 in the VH region sequence, M at position 20, K at position 40 and E at position 69 in the VL region sequence;
iii. A at position 101 in the VH region sequence;
iv. V at position 81, A at position 99_in the VH region sequence, I at position 20, K at position 40, S at position 69 and S at position 102 in the VL region sequence;
I at position 34,_V at position 81 in the VH region sequence, I at position 20,_K at position 40 and S at position 102 in the VL region sequence;
I at position 34 in the VH region sequence, I at position 20,_K at position 40_and S at position 102 in the VL region sequence;
I at position 34,_E at position 39,_V at position 81 in the VH region sequence, I at position 20,_K at position 40 and S at position 102 in the VL region sequence;
v. I at position 34,_V at position 81 in the VH region sequence, and K at position 40 in the VL region sequence;
M at position 45 in the VH region sequence, I at position 20,_K at position 40 and S at position 102 in the VH region sequence;
I at position 34,_A at position 65,_A at position 101 and S at position 106S in the VH region sequence;
I at position 20,_K at position 40,_S at position 69 and S at position 102 in the VL region sequence;
F at position 34_and V at position 81 in the VH region sequence;
M at position 45 in the VH region sequence, K at position 40 and S at position 102 in the VL region sequence;
D at position 39 in the VH region sequence, I at position 20,_K at position 40 and S at position 102 in the VL region sequence;
I at position 34_and V at position 81 in the VH region sequence;
E at position 39 in the VH region sequence, I at position 20,_K at position 40 and S at position 102 in the VL region sequence;
M at position 45 in the VH region sequence and K at position 40 in the VL region sequence;
I at position 34,_A at position 65,_V at position 81,_A at position 99,_A at position 101_and S at position 106 in the VH region sequence; or
vi. D at position 39 in the VH region sequence, I at position 20,_R at position 40 and S at position 102 in the VL region sequence;
I at position 34 in the VH region sequence;
K at position 40 in the VL region sequence;
D at position 39_in the VH region sequence, K at position 40 and S at position 102 in the VL region sequence;
I at position 20 and S at position 102 in the VH region sequence;
K at position 39 in the VH region sequence;
E at position 39 in the VH region sequence, K at position 40_and S at position 102 in the VL region sequence;
E at position 39 in the VH region sequence, I at position 20,_R at position 40_and S at position 102 in the VL region sequence;
D at position 39_in the VH region sequence, R at position 40_and S at position 102 in the VL region sequence;
V at position 81 in the VH region sequence;
D at position 39 in the VH region sequence and_K at position 40 in the VL region sequence;
F at position 112 in the VH region sequence;
F at position 112 in the VH region sequence and_I at position 38 in the VL region sequence;
E at position 39 in the VH region sequence and K at position 40 in the VL region sequence;
V at position 34 in the VH region sequence.

13. The polypeptide or polypeptide construct of any one of claims 1 to 12, wherein the VH and VL region sequence are linked by a linker.

14. The polypeptide or polypeptide construct of claim 13, wherein the linker is a peptide linker.

15. The polypeptide or polypeptide construct of claim 14, wherein said peptide linker comprises or consists of a G₄S linker, or G₄Q linker or repetitions thereof.

16. The polypeptide or polypeptide construct of any one of claims 1 to 15, wherein the polypeptide or polypeptide construct comprises at least one further binding domain.

17. The polypeptide or polypeptide construct of claim 16, wherein said at least one further binding domain binds to a cell surface antigen.

18. The polypeptide or polypeptide construct of claim 17, wherein said cell surface antigen is a tumor antigen.

19. The polypeptide or polypeptide construct of claim 18, wherein the tumor antigen is selected from the group consisting of BCMA, CD123, CD19, CD20, CD22, CD33, CD70, CDH19, CDH3, CLL1, CS1, CLDN6, CLDN18.2, DLL3, EGFRvII, FLT3, MAGEB2, MART1, MSLN, MUC17, PSMA, and STEAP1.

20. The polypeptide or polypeptide construct of any one of claims 1 to 19, wherein said polypeptide or polypeptide construct is a single chain polypeptide that is at least bispecific.

21. A polynucleotide encoding a polypeptide or polypeptide construct as defined in any one of claims 1 to 20.

22. A vector comprising a polynucleotide as defined in claim 21.

23. A host cell transformed or transfected with the polynucleotide as defined in claim 21 or with the vector as defined in claim 22.

24. A process for the production of an polypeptide or polypeptide construct according to any one of claims 1 to 20, said process comprising culturing a host cell as defined in claim 23 under conditions allowing the expression of the polypeptide or polypeptide construct as defined in any one of claims 1 to 20 and recovering the produced polypeptide or polypeptide construct from the culture.

25. A pharmaceutical composition comprising a polypeptide or polypeptide construct according to any one of claims 1 to 20, or produced according to the process of claim 24.

26. The polypeptide or polypeptide construct of any one of claims 18 to 20, or produced according to the process of claim 24, for use in the prevention, treatment or amelioration of a disease selected from a tumorous disease.

27. A method for the prevention, treatment or amelioration of a tumorous disease, comprising administering to a subject in need thereof the polypeptide or polypeptide construct according to any one of claims 18 to 20, or produced according to the process of claim 24.

28. A kit comprising a polypeptide or polypeptide construct according to any one of claims 1 to 20, or produced according to the process of claim 24, a polynucleotide as defined in claim 21, a vector as defined in claim 22, and/or a host cell as defined in claim 23.
